SEQUENCE LISTING (910 sequences)

SEQ ID NO: 1 - DNA, 1766 bp, Homo sapiens
ggcagctgcacggctcctggccccggagcatgcgcgagagccgccccggagcgccccggagccccccgccgtcccgcccgcggcgtcccgcgccccgccgccaggtgagccgggccctgggcgaggaggcgggagggaggagggaggggagtccagggcagccaggagtcgggcgagcctcgggggctgcagaatggggtcgcggccgcgatgcccctgaccctcgccggccccacccaggccgccccccgcgcgcggggctcccgcagcacagcctttctccggccctagcccaaatcgcccagaccaggcgcggatcccagcctggccagcaggcggcgggcgcggggcggcgagccggggccggacggctggagccagaaccggctgctctccacgcccccctctcggtgctgcccggaggccggactccgcctccaccgagcccccacccgccgggaagagctccgcggagtacagagcccattttctagctgtgtccactgaggctgaacggatccgcgcggacttggtgctccgtgctcgccccctagggccgggtccgccgggagcgccgccctccggagttgtccggccggcgcacacctgcccggccccgcagcgccccagctcacctctttgtctctcccgcagcgcacccccggacgctatggcccacccctccggctggccccttctgtaggatggtagcacacaaccaggtggcagccgacaatgcagtctccacagcagcagagccccgacggcggccagaaccttcctcctcttcctcctcctcgcccgcggcccccgcgcgcccgcggccgtgccccgcggtcccggccccggcccccggcgacacgcacttccgcacattccgttcgcacgccgattaccggcgcatcacgcgcgccagcgcgctcctggacgcctgcggattctactgggggcccctgagcgtgcacggggcgcacgagcggctgcgcgccgagcccgtgggcaccttcctggtgcgcgacagccgccagcggaactgctttttcgcccttagcgtgaagatggcctcgggacccacgagcatccgcgtgcactttcaggccggccgctttcacctggatggcagccgcgagagcttcgactgcctcttcgagctgctggagcactacgtggcggcgccgcgccgcatgctgggggccccgctgcgccagcgccgcgtgcggccgctgcaggagctgtgccgccagcgcatcgtggccaccgtgggccgcgagaacctggctcgcatccccctcaaccccgtcctccgcgactacctgagctccttccccttccagatttgaccggcagcgcccgccgtgcacgcagcattaactgggatgccgtgttattttgttattacttgcctggaaccatgtgggtaccctccccggcctgggttggagggagcggatgggtgtaggggcgaggcgcctcccgccctcggctggagacgaggccgcagaccccttctcacctcttgagggggtcctccccctcctggtgctccctctgggtccccctggttgttgtagcagcttaactgtatctggagccaggacctgaactcgcacctcctacctcttcatgtttacatatacccagtatctttgcacaaaccaggggttgggggagggtctctggctttatttttctgctgtgcagaatcctattttatattttttaaagtcagtttaggtaataaactttattatgaaagtttttttttt

SEQ ID NO: 2 - DNA, 1729 bp, Mus musculus
agcagagagaactgcggccgtggcagcggcacggctcccagccccggagcatgcgcgacagccgccccggagcccccagccgcggctccccgcgtcctgccgccaggtgagccaaggcagctgcgagggagcaggcgggaggggatgggaggtgatgggagcagagcccggcaggactaacctcgcagactgtatggcagggtcgaggatgccacgcctctggcgcccccccacccccgccccggctcccccaggaggcccctcgctcgagcggggcgccgtcagcccctcctctccggccctgagccctgatcctccgcccgggttccagtccccggcgtggccagtaggcggcagccgcgaggcgactagccaccagcggggacggccgggagtcgggcccctctccacgcccccttctccacgcgcgcggggaggcagggctccaccgccagtctggaagggttccgcatacaggaacggcctacttcgcagatgagcccaccgaggctcaagctccgggcggattctgcgtgccgctctcgctccttggggtctgttggccggcctgtgccacccggacgcccggctcactgcctctgtctcccccatcagcgcagccccggacgctatggcccacccctccagctggcccctcgagtaggatggtagcacgcaaccaggtggcagccgacaatgcgatctccccggcagcagagccccgacggcggtcagagccctcctcgtcctcgtcttcgtcctcgccagcggcccccgtgcgtccccggccctgcccggcggtcccagccccagcccctggcgacactcacttccgcaccttccgctcccactccgattaccggcgcatcacgcggaccagcgcgctcctggacgcctgcggcttctattggggacccctgagcgtgcacggggcgcacgagcggctgcgtgccgagcccgtgggcaccttcttggtgcgcgacagtcgccaacggaactgcttcttcgcgctcagcgtgaagatggcttcgggccccacgagcatccgcgtgcacttccaggccggccgcttccacttggacggcagccgcgagaccttcgactgccttttcgagctgctggagcactacgtggcggcgccgcgccgcatgttgggggccccgctgcgccagcgccgcgtgcggccgctgcaggagctgtgtcgccagcgcatcgtggccgccgtgggtcgcgagaacctggcgcgcatccctcttaacccggtactccgtgactacctgagttccttccccttccagatctgaccggctgccgctgtgccgcagcattaagtgggggcgccttattatttcttattattaattattattatttttctggaaccacgtgggagccctccccgcctgggtcggagggagtggttgtggagggtgagatgcctcccacttctggctggagacctcatcccacctctcaggggtgggggtgctcccctcctggtgctccctccgggtcccccctggttgtagcagcttgtgtctggggccaggacctgaattccactcctacctctccatgtttacatattcccagtatctttgcacaaaccaggggtcggggagggtctctggcttcatttttctgctgtgcagaatatcctattttatatttttacagccagtttaggtaataaactttattatgaaagtttttttttaaaagaaacaaa

SEQ ID NO: 3 - DNA, 98858 bp, Homo sapiens
gctcgggcgccgagtctgcgcgctgacgtccgacgctccaggtactttccccacggccgacagggcttggcgtgggggcggggcgcggcgcgcagcgcgcatgcgccgcagcgccagcgctctccccggatcgtgcggggcctgagcctctccgccggcgcaggctctgctcgcgccagctcgctcccgcagccatgcccaccaccatcgagcgggagttcgaagagttggatactcagcgtcgctggcagccgctgtacttggtgagtcgcggacccacgcggcagtcgccgacctcctccgagacccggactcgtgccctgtccgcagggtccctcccgcctcccccgcctctcgcctctagcctctcgctcggggcggaagtggcggcgcggtccgcgtcaggggcgcgcctgcgctgaccgctctcttgctccttttggtttaaaaaaaaaaaaaaaaaaagcatacttgagctgagcagctcatggttagcgcaagcgcagttagttctggagggcggtgccacagcggcttcttggggagcgcgtggatgcgccaccagcgttgcgcggcccgggtctgggggtccttgggaaagccgtgggggcgctcgggcgcgcgcccccgggccgcagcttcgccgtagctcccacgccgctgtgcttggttcttgctcgcggacagctctttctctggccgggtgttttgtggccgggtgggcagctggcgtccccgggagcggtgagggcgggggcgggcgagccgggccgcggcgtccgggtccagctggcgcccgcgcaggacctgtgggagcaggccgctctgggcgccgggccggccgccgcagccctctgcgaggcgaagctcgctctcctggcggagccgcggtggttgggggtggctcgtggcctctggcgagggcagcgctctcgggaccctccgtcttcattttcccccgtggcccagcagcgcccagcgtgggttcggcccccatagaaccttcccggaacgaaagaggctttgcttgtgttcgtccagtgatagcgtgtcattctctttatagttttccttactgaggtatactttccctacagtgaaacgcagctttcatgtgtgctcccagacgggtgtcacaaatgaatcccctcttgcaagccccctcaaatcaaaatagcctttccttgccctgagaaagtgccctcatcccctccccgggggcacggtgttccgatgcctatcgccctaatcgtgcctgttcagctcacatagatgcaatatgtagtataaacacctgtgattcgctttttttatgtcaactagactaccttttaattcaaacaaaactttatctcttttaaaatgatgtaactgctttccccttcgtttaatccttgtcgtattattcccaagattgaatttggtgacggttaagttaatgctgcttctgatgtttggttctctctgtgtaaagtaaaagacgggctttcctgtaaatggatgatcctgggtttttcccctttccatttaattagatggcagtgaaaaatgtgttcagaaagtaacgttttcttcttgaacacattagtgggaagtataaatattctgattcccatgatacagagtatgttagaatagggatctaagttttaaagaatgctggtaaggactcttccaattatagacaatctgaaacgcaaattctggtaggaatcttcatgtgagagagcctgccagctgcatcttctccagttacatttgttttagcagaggctgctgtctctctaagggataggttatatatcgaaatagatttgaaaagagaaaagtgaatataacttaattcagcttaacattttggtttaagaaatttaattttctttggtatcattaatatccttgaaaattcaatgagaattgtgattcctctttccagaaaaatgaacttaactcgcatccactggagatttcacatgcattttcaaaaagataaggaaatccttctatagattcttaccgaggaatcctgtagtttccttggttggtcaatgctcatattattttttcctagttgaaaacaagaccattagtttggtcagtagaaaccatggcatgcacactggtgttacagtaaaagtaggtcacaaaccaagaaagttagtaatggaaagcaagtcaggaagtttgagctatgtgttctcctgtggccttagaacatccattcagccattccacaaatatgcccggacccccagtggagttgcagcttttgctcctggccttggggattcagcagagaatgctcctggcaagtttcctaccctcgaggtgctcactctagggcttattcttttggggcctcattgtagttccatattttcattcattcgttcataacatgcttgttatgtgctagtgttagacactgaggattcttgttaaaagacaggagtcccagtttttaaggatgcagccctgtaaataggtaagctgatgaaaacgttaaagtgatacatgtaaagctagcttgaagcacagggtcttgtggagttgagttacctaggagtcctgtatttcccaaaaaggggtacacatgggagttctggtgaagcttctcgagggagggaggacctcacctaagtcttgaaagacattggccaggggaagaggacaagggaggtgaagagaattactgttccagcaaggagggagtgcacaaaggcactgagggaatgctgagagaaccacccacagtttagcctggagactggtgtgaagttgagatgctggagagtaaggcagagcctagatcagactaaggaatttgagctgtttaataggaactcattgtggaattgtaagtctggaaataccatgatcatatttgcttcttttttttttgacagagtcccgctctgtcgcccaggctggagtgcagtggtgcgacgtcagctcactgcaacctctgcctcctgggttcaagtgattcacttacctcagcctcctgcgtagttgaaattacaggcacacaccaccatgcccggttgatttttttgtacttttagtagagacagggtttcgacatgttggccaggctggtctcgaactcctgacctcaggtgatccgcctgcctcggcctctcaaagtgctgggattacaggcatgagccactgctctgggctgatcatatttacttgttagcaagatcgttctgactgctagcagactggatgaggaaagtgtaggcacgcagcacagggcagtagtcggttggagtaaacctgaagagacatgatgagcttctgttaggcagtggcaggagggatggaggggagatacagatttgaaatgaaatggtaaaggggtatacgttatgtacaagtttggttttgaaactttaattctgtttgaggtacctgcgagacactgaaatgaagctagttgaatgtataaatttggagttcgggagagagggtctgagtaaatgtgattttggaaatcatgaacacatgggtgatatttgaggtcttagacattgatgagatcatctagggaagatacagagcgtttggaaatatcaaaggccaaggacagaatcttgacagacaacatttaaaaggatgggtggagaaagaggaacttgcaaaaaagactggtgaggaacaaccacagaagtggggaggaaagcagtggagggagttgagggtgagcagggccacctgacaggtaagggccagcctgagagggccatgacattgagcagctagattaacagataccttaaagggagttgtgtcagcggactacacaagacagatacctggagttcaggagagaaggtaggaggaaatgcaaactaccaagtgtacactgctctttttagaagcccaacttgaagaagtggggagaggtagctaaaatgaagcaagattgtgtgacttctcacttaaacccttgaacggctttccgttgctcacagtgtaaattcagcccctcctccggctcacaagacctcatggcctgtgtctcttgcttgatttcccctatcactttcctccctcttttccactgtgttgtattcttggaagtgtgtgtgtccattctccccttagggcgtttgtaaatgttgttctctttgccttggcacaactttccccccttctctttcctttcttgcttaaactgcctcctactcatccctgaagtctcacttccttggcaggccttcttgacctgcccatgctgggtatgagcccctgttgcgtacttctttagcatcccaagacttcccctttcatagcgggaattggatgtcatacagcagcaaatgatttcgggaacacaatttgcattaagcaaaccactatcttgaaagtaacacagcttgagtaggaaaaaagtgtttaaggtggctttctgctttgtttagcacagcagtgggtgatgtttctgataatgtttttgaagagttgtgctttttttggtaatgctcatggagctgggaaaagaaaggctttgctcttgagcaaaaactgagtacaactaaaagctggacttgcttagtggtaggagaacttgataccttgttgaaattctagcgggaaaaatgaatttagtattttgagtctggaactctggtcccttattcctttttgctccttttgaaaagtgtcttttatttcatgagtttgataataggaggtttcttagaaatgaattattgggttggagcagaacagactctgttaatctgagtttatattttttattacttcttcagagtttcatgaaggcactgccagtgctttgcacagtatcaggtgcttagtaagcacttgaaaatatgtgctgaatgaatgactttttacgaggaaagggcaacttgatatatttatgtgattgcagggatgaacctaaagagagaggtggaggttgaaatagaagggatgattgttggagccagaaatcttagaaagcaagagtcaatgggaccaagagcacacaggtggaagaattgatcgtgaaagagtgctggccaggcggtggctcatccctgtaatcccagcgctttgggaggctgaggtgggaggatcacttgaggtcaggaggttagggctgcagtgagccataattgtgccactgcactacagactaggtgacagagtgagatcctgtctctaaaacaagaacagcaaaacaaagggtgcagttggaaaagtttaatttggtctgattgtgtgaggttaattctgactttttgagtaatgttcatgatccctttttggcttttccttttgtcactgctgattgctgtcttcatcaggctgagctttgagaaaggcagagagaaggaatggagtctgtgtgcctactgaggaaaagactaaaactggggaatcgcaaaagctatgttatgggttgtttctaagaagtgatggctcagaaggaatttggcttatttatttttaaattcctttcttttttaaagagttctcactctgttgcccagtctggagtacaagtgcagtggtgcaatcatagctcactgcaaccttaaactcctgggctcaggtgagcctctcacctcagcctcttggaaattggttgattaattagggtttataaacaatggaactcctgactgacttcatttttctttttttgaaaccaagtcttgctctgtcccccaggctggagtgcagtagtgtgatctcagctcactgcaacctctgctccccccaggttcaagcagttctactgcctcagcctcccgagtagctgggattacaggtgcccaccagcacgcccagctaatttttgtatttttagtagaaacggggtttcaccatgtttgtcaggctggtcttgaactcctgacctcaggtgatccacctgcctcggcctcccaaagtgctgctgggattacaggtgtgagccaccacgcccagccacatttttctttttctttctttttttcctttttctttctttttttttctttttctttcttttctttttgagactgggtctcactttgtcacccagtctggagtgcagtggcgtgatctcagctcactgcagtctcgacctcttgggctttagacatcctcctgcctcagtcccccacgtagctgggcctggctaatttttttgtattttttgtagagacagggtttaactattttgcccaggctggtcttcaactcctgagttcaagcaatctgtccacctcagccttccaaaggcttaggattacaagcatgagccactgtgcctggccatttttcttttttctttttctttttttgagacagtcttgctctgttgcccaggctggagtgcagtggcgtcatctcggctcgctgcaagctctgcctcccaggttcacgccattctcctgcctcagccttctgagtagctgggactacaggcgcccgccaacactacaggcgcccgccaccacgccaggctaattttttgtatttttagtagagacggggtttgaccgtgttagccaggatggtcttgatctcctgactttgtgatccacctgcctcggcctcccaaagtgctgggattataggcatgagccactgcgcctggccgccatttttctttattagaaatttgtagttcaaatattgctagagaacaatttttaccatttgtgagtcagctaatttttagaagtcagatatcaagtgcacactgttcttctcatacttacagcggggcagccagttaggcaggtgaatcaaagctgtcttattctaaaacacttcccttgcctactagttacacattctcctgcctgctgtctaatctcagtgcttttacatggttgcacaaactctgaattcccttgaagattggctactctctcacaccttggagtcttcatgtgttggatatactccatgcctaccatatctatttgggaaacatcttcaagactagaccgtagtctccttgggcaaagttaaatgctcccccttctctcatttcttagttctctatattcttttattagaatatgaaccacattgttttgtaactgtctacttgtctgttctgtgtagctccttgagggtggtctttttttttctttgttttcccaccacctacctacgggacctgaaacatgttagatgctcagtaaatattcagtaaacacaggaaaactgttcccttttaaaatgtcagcaccttagtttattccaaacccctcatttatggctgaggaaacactgggccaatacagtgttgtgacttatctgatgttacaatatctgctagtgtcataactgggactcaaatccagtccttctgattctcgtttcagcactgccctaatttacccatgtgactacctgtagtgctttcttcttatgatctatttgagatcagtggcacagagggaatcgtcttaattttgatggctaggagggagtggatgtgttataataggtagggtcattttgggacttgaggcctttaaaacagtgtctgggatccagggatcagacttgaaaggtatttgcaaatctcctgaaactgctaaattttatgtacgtgtgttcccaggtatttcgtttctaggtgattctgtagcttttgtcagtttcttagtggatttgtctgtgatccctcaaaaggtaaagaccactgcttgaagggaaagcagccttgggatgggtgccccaatggggaatgtatacctagaaatcatattgttttgcctttgacatgtgtttatagattactgatatctataattacggaaatccagatagttgcgtttgtctcctagcttttctagttgcctcttttttgatttcttgattttaaccagaatcttctttttttcttgagaaggggtctcattccgtcacccaggctggagtgcagtggcacgatcgtaggtcactgcagactcaaaactcctggctcaagtggtcctttgcctcagcctcccgagaagctgggactacaggcacacaccaccacgcctgacaaatttcttcttcttcttcttctttttgttgttgttcttgttggccaggctggtctggtcttgaactcccggcctcaagtggtctgctcaccttggcttcccgaagtggcgagattacaggcatgagccaccatgcccagcccttaaccagaattttgttttgttttgttttttgagacagggtctcactctgtcacccagactggagtgcagtgatgagatcacggctcactgcacagatgcacgccaccatgcttggctaattttttatgttttgtagagacaggttctcaccatgttgtccagactggtctcgaactcccgggctaaagcgatcttccggcttcacccttccataatgctggggagccacagtgcttggccaaccagaatcttaaaagccctttttactttttccagatagttactctgcagagaatttctgattcccagttggagaactgttacagctctctcagtcccctgctgttcttttagctaccttgtctttgtcctcttacctgtccactctctactccccttcttccctttggtctccaaatagtccagataaaccagagagatggacagataaggtctgggtacgcgaaattaaaattaacatagtgtgctcttcactttcttgaataaacgttacgtatgatttgtttcccttggaaatcctcatctagattctctactctctattccttaacttaatgccccattaaggtagttttttcttaatggaaagctgttgagagtcagaggatatttaaaccaactaataaactgaacttttaataataccacccttactttggataagagtgattagccatatactaaatcctcaatcagtttagcatgtaaaatttaagacatagcagctgataactgaggaagagatacatttccacaggctaactttaaagaatataagctctcactttcctgagtagaagaaaactaaacttttctgttctatgctttttttttttttaatttttatttttattgatcattcttgggtgtttctcacagagagggatttggcagggtcataggacaatagtgaagggaaggtcagcagataaacaagtgaacaaaggtctctggttttcctaggcagaggaccctgaggccttccgcagtgtttgtgtccctgggtacttgagattagggagtggtgatgactgttaacgagcatgctgccttcaagcatctgtttaacaaagcacatcttgcactgcccttaatccatttaaccctgagtggacacagcacatgtttcagagagcacagggttggggggtaaggtcacagatcaacaggatcccaaggcagaagaatttttcttagtacagaacaaaatgaaaagtctcccatgtctacttctttccacacagacacggcaaccatccgatttctcaatcttttccccacctttccctgctttctattccacaaaaccaccattgtcatcatggcctgttctcaatgagctgttgggcacacctcccagacagggtggtggccgggcagaggggctcctcacttcccagtaggggcggccgggcagaggcgcccctcacctcccgggtggggcggctggccgggcgaggggttgacccccccacctccctcccggacggggcggctggccgggcggggggctgacccccccacctccctcccggacggggcggctggccgggcggggggctgaaccccccacctccctcccggacggggcggctggccgggcggggagctgacccccccccaccttcctcccggacggggcggctggccgggcagaggggctcctcacttcccagtaggggcggccgggcagaggcgcccctcacctcccggacgaggcggcaggccgggcgggggctgatccccccacctccctcccggaaggggctcctcacttcccagtaggggcggccgggcagaggcgcccctcacctcccagacggggcggctggccaagcggggggctgacccccccacctccctcccggacggggcggctggccgggcggggggctgaaccccccacctccctcccggacggggcggctggccgggcggggggctgaccccccaacctccctcccagatggggcggctcgcctggcgggggctgacccccacctccctcctggacggggcggctgccaggtggagacgctcctcacttcccagacagggtggctgccgggcggaggggctcctcacttctcagacggggcggctgccgggcggagggtctcctcacttctcagacggggcggttgccaggtggagggtctcctcccttctcagatggggcggctgggcagagacgctcctcacctcccagacggggtcgcgaccgggcagaggcgctcctcacatcccagacggggcggcggggcaaaggcactccccacatctcagacgatgggcagccgggcagagacgctcctcacttcctagatgggatggcggccgggcagagatgctcatcactttccagactgggcagccaggcagaggggctcctaacatcccagacgatgggtggccaggcagagacgctcctcacttcctagacggggtggcggccaggcagaggctgcactctgggcactttgggaggccaaggcaggcggctgggaggtggaggttgtagcgagccgagatcacgccactgcactccagcctgggcaccattgagcactgagtgaaccagacaccgtctgcaatcccggcacctccggaggccgaggctggcggatcactcgcggttaggagctggaggccagcccggccaacacagcgaaaccccgtctcccccaaaaaaatacgaaaaccagtcaggcatggcggcgcgcacctgcactcgcaggcactctgcaggctgaggcaggagaatcaggcagggaggttgcagtgagccgagatggcagcagtacagtccagcttcggctcggcatcagagggagactgtggaaagagagggagagggagaccgtggggagacggagaccgtggggagagggagaggaggcagagggagagggagaggcttttttttttttttcttgagacggagtcttactctgtcaaccaggctggagtgcagtggcacgatcttggctcactgcaacctccacctccctggttcaagtgattctcctgcctcagcctcccaagtagcagggactacaggcatgcgccaccacacccagctaatttttgtatttttagtagagatggggtttcgctgtgttggctgggccagtcttgaactcctgacctcaggtgatccacccaccttggcctcccaaattgctgggattacaggcgtgagccaccgcgtccagcctgttacattcttaattggaatagcattgccctgcagtgccattatctgaaatagcttttaagaatgaatgtaatgccattttaaatgtatttaaatgttgtgccgatttaatgttctatgtacggtacagtactactaatggttcaagaagttgttttgacttaacagtgaaatccttgcttttaaagatcatagatacctttagacacctttaagatgaaacatgtttatgagaggaaatgtccgcacaaatacaatcccccgcaaacttagcatgtggttttagtaggttcataggcccacctggtgatccctaggataagaaattgtggtcaagaaactgatttcaggggccgggctggtggcttacacctgtaatcccagcactgtttgtctcaaaacaaagaaactggctcaaggttggatacttttcaaacgagaggaatggaggaagggaatgcaatgaataacttttcaaatccagtatgtctgatttaatatgccgtttgctctgtcagtttcatttggttcgtgtcactgatcatttataaccttttttcaaactgctttcattcccctaaggaaaatctgatactgaattattttatttcttctttcactatatttgaacggttttatagtctgaaagctatcaatttctcattacagtttacttcatagtacttctaggaattcttcctgtggtttgaggtaaggctacaggattcctagagtttgtacccctctagtcccagtaaaggacctggtacatcaatcagggtagccctagaaagttggagtaacttcgtaacagtgggaactgcttctgttctatgttttttgtgggttttttatttgtttttgtttttttgtttttttgttttttttttgagacagagtcttgctctgtggccccaggctggaatgctacggcacgatctcggcttactgcaacctctgcatgctgagttcaagcgattctcctgcctcagcctcctcagtagctgggattacaggcgtgcaccaccacgcccagctaattgttgtatttttagtagagacagggtttccccatgttggccaggctggtcttgaactcctgacttcaagtgatcagcccacctctcagagagctgggattatacatgtgagccaacatgcctgccctgcctcattctttatactacagtaaagtcccggttgggcaataatctaaacataaaagaaatgaaaccataaaattatataagaaaacaatggagaaaattttttgtaatcaacagagtgaaaaaggattttttaatgccatgtcacagaaacctagagaaaaaaggtttaagtttgatttcataaaaatttaaatttttgacataaataccataaaggtcaaaagataaaaaattttaaatattttcaatatataacaaaaactaaactttcttaatatgtaaagaactacatatcaaaaagagagcggaaatacagatggcttttaaaaacaggtaaagctgttcaccttcacaataataacaatttcaaattatgactataataataccatttttcacctatcagacaaagatgaaaaagttcattaatataattgtgtaggtgaagctcaggcactttcttatattattggtgacagtatacattgatatagcttctttgaaaggttattcatagcagcattatttataacagctaaaacctagaaataacctatgtgtcccattatagatgactggctaagtgatgaatactattaaattgttaaaaagaactcattatctatatggaagtacttttaatatatactaagtgaaaaatcaaagtgcaaaacagtatgctaatatctgttttgtgagggtgggatggatacttggatggaggaagacagacaggcagtctggcttatatatggatagaatgtctcggaaaatatacaaggcactggttacagttatcacctaagggaaaggattgagggagaggagaatgtgacttaactttttcactatatacccttttgtacggtttaatttttgccaagtccatgtaatccatgtatacatagggagcatatgtatataaatatatgtaaatataagtaaaaaaatatgtatgtgctgccgggcgcggtggctcactcctgtaatctcagcactttgggaggccgaggtgggcggatcatgaggtcaggagatcgagaccatcctggctaacacggtgaaaccccgtctctactaaaaacacaaaaaaagtagccgggcgtggttgcgggcgcctgtagtcccagctgctcgggaggctgaggcaggagaatggtgtaaacctgggaggtggagcttgcagtaagccaagatcgcgccactgcactccagcctgggcaacagagtgaaactctgtctcaaaaaaaaaaaaaaaacaacacgcttttctctctctctctctctctctctctctctctctctctatatatatatatatatatatacacatatatatatacgtatatatatacatatatatacgtgtatatatacatatatacgtatatatatgtatatatacacatatatatacacatatatatgtatatatatacatatatatacacatatatatgtatatatacacatatatatgtatatgtatatatatacatatatatatatatgctaaagttaagtactggattcctatctgtatctgctacctaactgcagtataaatcttggaagttgagtctgttttctcatttgtaaccacctcttagataacacacataaaagcacatagcacatgcctgacacacagtagggttcagtgacagtgagctgttttagtctctaaactctcttcctcaaagagagcttctcaaagaaaaaaaaatgtagtgaagaaaagaacttactccaccagggatcttctctccccagccagtggcaggcatattaagggctataagtaagtggttattttaccatcctaaagtaatggtgtttcaaagtatgcaagattcttcatacttcactgtgtccttttctgagcatctttgtagagagtatatgcacaaacatctgactaacaaagggaacacttttcatcatataatttaggattcacttagagacctaaagtataaagaaagaaacttaggtatgttagcccataaggagaaactcattatcaatttgggattatcttaaggtcctaccttataaaattaaaatttaacagaagattattctttttcaatttcctaagttcttatttgctactacatcgtagcagttcgagggagaatttggtctaacgatgtaatgttttctgtatgccatacatagaacaaagctatgtacgaaatactaaaaatattatttgacattatttaataaaatattataactatttcaccccgataatgatggaaattaagttgcagtaatatagatttttaaaataaaaacttcaaaaccaggcgccatggctcacgcctgtgataccagcgctttggaaggccgaggcaggcagattgcttgagaccaggagtttgagaccagtctaagcaacatagcaaataaaaataaaaaattagccaggcatggtggtgtgcctgcagtcccacctacttgggaggctgaggtggggggatcacttgagcccctgaagtcaaagctgcagtgagctgtgatcacaccactgcattccagcctgagtgacaaggcaagactttgtctcaaaaatataagtaaaataaaattaaaattttacttgtttatttttttgagacggcgtctctctttttcccaggctggggtgtagtggtgtgttctcggctcaccgtaacctctgcctccggagttcaagcgattctcctgcctcagcctcccgagtagctgggattacaggtacgtgccactatgcccagctagtttttgtacttttagtagagacagggttttgccatgttggcaaggctggtctcaaactcctaatgtcaagtgatccgcctgcctgggccccgcaaagtgttgggattacaggcgtgagccaccgcacccagccttaaaactttaaaattcagaatatgagcctttgttttatagcttcacttgttaaaacaaatcaggagctgggtgtggtagctcacgcctgtaatcccagcacttggggaggctgaagcgggcagatcacctggtctcaatctccctgaactcgtgatccgcccgcctcagcctcccaaagtgctggaattacaaacgtgagccaccacgcccggcctgaagtttgaagtttttaaaaagaaagagttgttaggcataattgtgtaaatagaggtaaaaattgacagcaagaaaggcagtagaaaactatgacaaatgaaggtgatggctcacgcctgtaatcccagcactttgggaggccaaggcgggcggatcacctgaggtcagtagttcaagacaagcctggctaacatggtgagaccctgtctctactaaaaaatacaaaaattagccaggcatggtgatgtgcacctgttatcccagtcacttgggaggctgaggcaggagaattcacttgaacccgggaggcggaggttacagtgagctgagatggcgccacttcactccagtctctccagtcagtccagacagagtgagactcaatctcaaaaaaaaacaaaaaacaaaaaacaacaactatgacaaataagtgtaatttcccaggactattaaggtcttgtaatcccatgcactggaagcttttgttgttgttttctgatttatcattgaagtgaccaaagcgcagtggtaagaacaagcctttgtgattacttcacactagagaagggtctttcccacatatttgagacttaaggagttgatagtgaacagtgtaagcggccacacaggtgtgggttaagaactgatgtgtgtattcactttctgtaaagatgaccaaacatatgttgttagacaaacgaaaaattcaccccaagatcattgagggcatgcaaaatagtatgaggtagaggcagctgtaattcaggtgtccaaatacaagagcaggtggagaaaagaaaagaaactgagaacagtaaagagtttttaaagttataacacacaattgttaaaatagaatgttcagtaccaagtagcagagtagataatgcagaacactgaggtggcttgagaaaaatctctcagaactcaggaaagaggtgtgcgaaatgagcgttaggagagagcacttccccaagtctgtgatagaaattcctgaaagggaacagaggagactgaagcgaaactctattaagcgacaaggaagactcaaatggagaagctaacccagttccgatgaagacttggcacatatctataatgtattttttttgaagtcagccaaaagaaagtttaaatttgaggtacatctgaaagagtaagcaatacagaaaaatcttgacaacatctgaggacaaaaaaaaaagtactgatactgttgagtgtgaggcaaaacctcactttcagtctttcttgagaaatattggcaaaatatgcccttaatacaggagttccatttttatttcatttatttatttatttatttatttattgagacaggttctcactttgtcacccaggctggagtgcagtggtacagtcactgctcactgcagccttgacctcctgggctcaagtgatcctcccagctttgcctcccaagtagctgggactgcaggcacgcatcactacgcttggctttatttttatttattatgtgtttatttttatttttattttttgtagatacgtggtttcaccatgttgcccaggctggtcttgaactcctggcctcaaatgatcctcctgccttggcccagagtgtttttgttttttttttttgtttgtttgttttttgttttttggagacaatcttgctctgtcacccaggctggaatgcagtgagtggcgcgatctcggctcactgcaaccaccgccttctgggttcaagcaattctcctgcctcagtgtcccgagtagctgggactacaggtgcccaccaccatggccagctaattttttgtattttttagtagagatggagtttcaccatgttagccaggatggtctcgatctcctgacctcgtgatttgcctcggcctcccaaagtgctgggattacaggcatgagccaccacgcccggccgagatatatttcacataccataaaattcacctaaaaggtacagtttagtggtttttagtatattcacagctgtggaactaaatcttaatttcagaatattttcaacaccccaaggagagactgggtacatacagttattctccattctcacccacatcccaacccctgacaaccactaatctactttctgtctccagatttccctgtcctggtctcataccatgtgtagtcttttgtatctgtttttttgttttgttttgttttgttttgttttgttttgttttgtttttgagaggagtctggctctgtcgcctaggctggagtgcagtggtgcaatcttggctcactgcaagccccgcctcctgggttcacgccattctcctgcctcagcctccccagtagctgggactacaggcgcccgccaccacacccggctaattttttgtatttttagtagagatggggtttcactgtgttagccaggatggtctcgatctcctgacctcgtgatccacctgcctcggcctcccaaagtgctagggttacaggcgtgagccaccgcgcccggctggtttctttaacttagcataatgttttaaagtttcatctgtgttgtatgtatcattactttgtttcttcttctaagttagtaagattccattatatagatatataccattttgtttatccattcaccagttgatggacaaatgattgttttagctattattaaaagtgctaccatgaacatatgtgtccaagtttttgtgtgaatgtatgtattcgtttctcttgggtatgtgccttggagtagaatagctagttcatatgttaactctttgttcagcatttgtttttctaaagtggctacatcattttaacctttagttttttgttttttggttttttttttgggagtggggtggggctggagtgcagtggcgttatctcggctcactgcaacctccgcctcctgggttcaagcaattctcgttcctcagccttccgagtagctgggactacagacacatgccatgacgcccagctcactctttttgcccagctcactctttttattttagtagagacagggtttcaccatgttgcccaggctggtctcaaactcctgagctcaggcaatctgcccacctcagcagggtttcaccgtgttgaccaggctggtctcaaactcttgacctcaggcgatccacccacctcagccttccaaagtgctaggattacaggcatgagccactgtgccccgcctctctcttttttttttttttttaataaagacagagtcttgctctgttgtccaggctggcatgcagtggcgtgatcacggcttactgcagctttgacctcctgagctcaagtgatcctcccacttctgcctcccaagtagctgggaccacagatgtgtaccaccatgccttgctaattttaaaattttttaatagagatgggggtctgtctgtgttgcccagactggtctcaagtgattctcccacctcagcctcccaaagtattgggattgcaggcatgactcacagcgcctggcctcagtttctttcaagaccatcctataacaccaagttagtttgaggattctatgaatgtctgcttaaagatatccttctgaattctgtgattctcttgaaaggcttgttgcaatgaaaaacaaagtataatgatgactttatgtgaagtatacttttggaatttcttaagcagaagtcctgagacttttgagagtccctgtatttaaacagtaccatttaggcacagttactgcttatgaaatgaagagtagctagatgaatataaaaaatcttactcttacatttcaacccagcattatttgaacccagcaaaattatttctatagtagaatcaatttagttccttattttaagccatgcatatatatctcaaagcaaatttcaagagggattaaatgttattatgtgaaaatattaggaggtgaaataataggagaaagtaaacacattgaaagtaggtatgttcaggccaggcgcggtggcttatgcctgtaatcccagcactttgggaggctgaggcgggcagatcacgaggtcaggagatcgagaccatcctggctaacacggtgaaaccccgtctctactaaaaatacaaaaaaaaattagccgggcgtggtggcgggcatttgtggtcccagctactcgggaggctgaggcaggagaatcgatttaaccctggaggctgaggttgcagtgagccgagattgcaccactgcaccctagcctggcaacagagcaagactctgtctcaaaaaaaaaaaaggaaagtaggtatgtttacaatttaaaatctttgggcatggagataagctataaggccattgatcagaatggaccatttcataatgttccaaaaattagactactttggaaaaaggtggtgtgtggtcttaatgtaggcaatgcacatgtgtgtttaagtttcaaatacagctaaggagctaagtggaattttttttttttttttttaccaaatagcgtgtattaataatagctcagttccatttactgatttgtttttcctttgtttatgtcatatattatctttccaagagattgtgagtttatgctactgccaatttggtatgagggcctgtttttaaaagagctgcatgatattcacttggatgtaccacgatttattcaaccagtcttttctttgtcattagacatttagtttatttaaagcctttcaacattataagtaactgtgaaagatatctttgtgctatgagttgcctacattttggatcattgccactaagaaagaaattaactagtgactcaattattccttgctttttttgagcaaaaatcatattaaaaataattagatttaaaatttttggttaaattatactttaaagcaaagatccttgccatttagatctagtttggtattaaatgagaacggtggttctctactgtgttggtgggggatatgttcatgatggattttgctgtggaaggacatttggcaatgcctggagagagttttggttgttacagatgcagggaggggtgccactgacatctaatgggtagaggccagggatgctgctgctaaacatcctgcaatacacaagacagtccccacttgcccccccccccccaaaaaaaattagctagcctaaaatgtcagtagtgctgaggctgagaaaccttggattaacaggtttcactgctgatgggaaacttttttttttttaacagggccttactctgttgcccaggctagagtgcagtagcaggatgctagctcactgcagtcttgaactcctgagttcaagcaatcctcctgtctctgcctcccaagaagctggaactacacatgcacaccaccatccccagctaattaaaaaaattttttggcagagctggtgtcttactgtgttgcccaggctggtctcaaactcctaccctcaagtgatccttccagctgggcctcccaaagcaccaggattgcaggtgtgagccactgtgcccagaggaaacattcggaatctgtgatagaaggggaaggttttagctccagaacacaagtagagctggctttgccttatgtgatagacctagtgaatagctcagctgtctgctttcctaatgccatttagacagatactgtatattttattgcttatgcaatgtgcttataaaattgtttatttttgtgtcttgggtattcctgaaaatgcctcatcagtgtcagatctagttgctgttgagatttggttttgctgcgtatattacagacctgatttagctacatgggaaagaaaattaatcaatagctgatttaacaaccaggagtttatgtttctcatgtgatatgaattctggagagagacttaagggctgttactgtggcttcttgatgtcacccatgactcaatgctggtatttctgcttacctttcttaggaatgtgcattttaccttgctgtcacaggatggctcctgcctgttcccacccctcacccccggcagcaaaacctatgttccaggcaggcagcagtgaggacaaaggtacctggagccagtcctctttgtcagcgttttcctgcagccccaacccagtggtttctgcttattcccattggccagactgtcacaggaccttagaggctgggacaaatagttttcagctgggctcattgtggtctgaaaacaaaagcagttacctttagtaagaaagaaaggatattggggctgggtgcagtggctcacgcctgtaattccagcactttgggaggctgaggcgggcggatcacttaaggtcaggaattcaagaccagcctggccaacatagtgaaactccgtccctactagaaatacaaaaattagccaggcatggtggcgcatgcctgtaatcccagctactcaagaggctgagacaggagaatctcttgaacccgggaggtggaggttgcagtgagctgagattgcaccactgcactccagcctgggcaacagagtaagactctgcctcaaaaaacaaaaaaaaaaaaagaaagagaaagaggatattggaataaacgctaatatgcacctttgggagctgctttttgaaatattggttcttgtaagttcactgaaattttttttataaagtagaacaatattggagatttgaaagaatggactcaatgcaagagatcttattagctgagaaaatgtatttatgttatgaatgtatttcttaaccccaagaacttggctgttgtattgtgttttgtaaattttaaatctccctactactatactgggggccttaccctcttttgtatagtgcacacaaagaagtgtgtttctctgggctaaaaatagaaatctgcacctgacagatggccttaagtgtggaaaatgggtgtgtgtgtatcacagaagtggaagaagtggagggagtcctgctctgcctgttgttttaagcctcccctccctgcaaatctatatctttcctatacactagtaatggtcaataggtttttgttgttgttgttgttttaatcccatttacagtagcgacaaaacttatcaggtatctaagaataaacctttattatacatttatgctaacctttagcatacctttatgaagaaaactacaaaattaattaaaatatcatgaacggcaatgcaaatggggagaataaataggggacaacttgccttccagatgcattgattacaaagctgtagtaactaaaatttgatactgtctcaagggcaagcaaatgcttctgtgaagtagaatgaggatctagaaacagactcctgcgtgtgctgaaacttggtttatgatagagcaggtgttactgataaatgcagaaaccaataaatggtattaagtcaattaactttccatttggaagggataaaattaaatttctcttccacgtgcaggctaataacagtgtcttatatcttgcaaaagtacttaccagtccattcacttccagaaaaatggagaagtttcatattagaaatttgtgcatcatgtttcataaataaaaacttcagatactcggctgatggtgttggctcaaacctgtaatcctagcactttgggaggctgaggcaggtagatgacttgagcccaggtgttcgagaacagcctgggcaaaaagtgagaccctatgtctataaaaacaaataaacaaaatggcacctaacttttaaaatgctttttcttacatgtaataatgtgagcaaaagcataatttaatttttcctattaaaatgctttttttttggtttctgttacatttctgtatttacttgaattcttagaacactgtttcctgggagatcaaacctggccattctggatacatcagtagtgagagcttagtgcggacatagccttacagaatgtcttctatgttgaaatgcagagaacccgaaggcgcctaggatgagcctcaggcctgctggttgttgcagtggcgcaatctcagctcactgcagcctccgcctcctgggtttaagcgattctcatgcctcagcctcctgagtagctgggattacaggcgctcgtcaccaggcttggctaatttttgttttgttttgctttttccgcctcactctgtcacccaggctagagtacagtggtacgacctcggctcactgcaacctcagccacccggtttcaagcgattctcctgcctcagcctcccgagtagctgggattacaggtgcctgccatcgcgcctggctaatttttgtagtttcagtagagacgaggtttcaccatcctggccaggctggtcttgaactcctgacctcgtgatccacccacctcggcctcccaaagtgctgggattacaggcatgagccaccgcgcacagcctaatttttgtatttttttggggtttttttttatagagtctcactctgtcgcccagactggagtgcaatggcacgatcttggctcactgtaacctccacctcccgggttcaagcaattcttctgcctcagcctcccgagtagctgggattacaggcacccatcaccacgcctagctgattttttgtatatttagtagagacggggtttcgctttgttggccaggttggtctcaaactcctgacctcaggtgattccccgcctcggcctcccaaagcgctggtattacaggtgtgagccaccacacctggccaaatttttgtatttttaatagagctgggttttcacaatgttggccaggctcgtctcaaactcctgacctcagggaatccgcctgcctcggcctcccaaagtgttgggattacaggcgttagccactgcgccaggcttgttgctgaacatttatggctctagaaataattttttttttaaggagaagtaatgacaaacatgatagaaataaaatttgaggatatttctgggttcaaaataaagtaacagttaaatagcgtgaacatgaagaaataaatattttcaggtagaaactccaaaatttaggtagatgctgtgagttttagtcagaatttgaatatgatgtggctgaccatagatacctccattgcattaattctgcttcataaagtgtttgatgcttgcttgtaggtgttgaatagagcctaattagcagtaagtaaacagatctagagcacagactttggcatctgaaggaccttggttcaatgctgggactgccacttactagctatgggatctttggtggatttttaacctttgaaagcctttcatccctcacagatataatggggaagaaaatcgtttccattagtattgtgaggattaaatgattaattgatgtaaacaatgtgctttccatagtacttggcatatcataagtgcttcatgttattttacgctggctgggaagataagttttgctgtggagaatttaagagggatattaaaatatatttttgttattttaaggaaattcgaaatgagtcccatgactatcctcatagagtggccaagtttccagaaaacagaaatcgaaacagatacagagatgtaagcccatgtaagtacttgtgggtttgtgtgcatgtgtattttttggtttgttttaggaggcaggatgtagtgaaaaggagggcttggggtcagtgttgataaatacagtaatttttttcctatttacgtaagacacgttctttaagaatttaaaggtgtagaatagtggaaggaaaaataattacccataattatttcatccttcaaacataggcaccattattttggtgtatttcccacaacctgtttttcttatgcacagttttcctttctttaaaataattgtaattatagatgtctatataatatccatatgtatttccttttcatatcattccatcccctttgtaagttttgcttaaaatagttattggtgtagtttggtttttttccttccccaagttataagagtagtatgtactcgctgtagaaaacttagaaaacagagaagagtataaggaacaaacttatttaaaaataaataataacttgggtttatattctttggagtttttaaaaataaacatgtactttaaacggaaaattagaatcttgccacatactcatttgtagctcttttcttagcagtacattgtaaacttttttttattataaacaatttttaattgctttgacagtggctttggatataccactcttttgcataagttccctgttgttggacatttagattatgatgtatacacacacagacgcacacaattgtgtgaacaattataatactagagttctttggtgtattcatcatcatgcggtgattttattttttgttctgattcatgcagcttattcagatattaaattgggcctccttcgtgcttcagcatatttccctatgtttgactttagtgggctaaacataggttttctctttagggctccctgttggtagggacttgaaaaacagtatcagattgatcacattctggtttttgcttgttactgattgttcgcctgttgactagggtatggtgctgcctgctaagatctcctgtgtaggttgaaccatttcactgttcaaggacacagactgtatacctttatatacattgtagtcattggttatagataagatatttagggtctggatgtaccactgaaaaatccatcagttactggaaaaaaatccttatttttattcttacatgaatagaatgtttttcttatgttgaccaaatacatttccaagaagggtgtattggctgattttgggcaaatcaaatgagaagcagtctctgaaaatctttcatgtttgttataaattactattaaatactatgtttctcttaatgcttttttctttactaccagttagaagtgttcttaaagacaagtagctagaagagtggttttccattttcttcagttgcagcacacttgaagatttgacccacttcctctgaaacaggtggctcatcacggtggcagttccagtgtgggtgtgcgttcagggaatagcgggtgtgggcagtaattctgatacttaggtgcccttcccctgcctccaaatctctgattgagaatcactgagctacagagaataattctctaatgttaccatgagttttgaagacagtattttccagttgtacaccttgctttgatgcctttaattttttaaaaacttttgattttgaaaattgtagatttggatgcagttgagaaataatacagaaagatcgagtatacccctcacctagtttcccccagtggtaacatcttgcataactacagtccaatatcacacctgggaacctgaaacaatacagtccatccaggtttcaccagttttacatgcactcatttgtgtgtgcgtgtaagttctgtgcagttttatcacgtgtagattcctggaatcaccaccacagtctagatgcaaaccagtggagcattcccatcacagggtcacttgcactgcctttctggggccacagtctccttgcttggatcctccttccctaacctggtgttgcctttaatttttatatatttgttgtaattgcaattattggatgcactacactacatttgagtcccctaattagttttatcgaattgatctaaatgggatttttttttttttttttttgagatggtgtctcactctgttacccaggctggagtgcagtggcgtgatcttggcttactgcaaacccctgcctcctggattcaagcgattctcctgcctcagcctcccaagtagctgggattacaggtgtgcaccaccatgcccggcttatttttgtatttttagtagagatggagttgcaccatgttggccaggctggtcttgaactccttacctcaagtgatccgcctgcttcagtctcccaaagtgctgggattacagtcatgagccaccatgcccagctgataattttattttaactggaattgagatgttttattttaaagtaaaaacatttgcaacttaagttatcctcaagtatatacatcaataaaacttatgatttttctcctcagcttttagacttatgttgtatacctgcagttgaaggctgcagggagactctttaaaatatgcgttatcaggcctccctccatcatcattgggaccaggctcttcctgtatgccttctcattgaaaagcacttagccttgagagaaccaagcttccctcctgcggaccatgcagtggttgtcctctggagctgtgctctgctgggagcagcccccagtgcatgttctccttactcttcttcttctttgccccctccctccccacacatgcctccctagtaagccatgttctcctgatctctcagaaagagactgctcattcttgcttctcacgtagccacaggattttctagtctgagccaatatgcttcagcctgcaaatggaaatgatctcttccaaggcttgtgctggccagcacatccccacaccgccttggcacctctcctctcctgacttcagtcattcactgtctttctccttaccctctccttcccaccttcctcatcctgggtgacttcatgtctctgggaaagaactacggctcattgcgctagagttcctagacctgttcagctccgctgacattgacctttagtcatcttcacctctgaaatctgaaactctgctgccttgagggctactttctgtcattccagctttcacttgcttactcccactgtacctgctctttgacttcagcaggatctttagttgactttttcatgtattgtgaagttggaaatgtattgaattgaaatttcgattaaaaaatgtagtgaagcatattactttgtcctctaacctaactattaataactttcttgttgtttttatatttaaaaaataactctggattttttaattccaccattttgtgaagctccaacttaggaaaaatcagtcattcatcctctctgcactttgggcaaatgagcgtgtgccttgggtggactccagtgttaggtgggcttggggttgccttgctgtcctcatgccctcgtctgccccactgccaccgtgttccagctctgcaccgagtaaattatctctcagagctcctcaccatgctcaggatatcgtcctgctttcaattctacagataaaaattgtaaaaatcaggaaacaccaccttagcttcatgttctatcacctggaaactcactttcttttgcccctatccttccctaaagtcccaggagagaagcctcccctccctctgtcctagccgaatccttcgtccccatcctcctcatgggccctgctcttcatcatcactgctttttgtgtgttttcagcctctttctatctcataacttcttctcttttgtataaatatggttcgctctcttttgtctcaacccttatatcctaccccagcccccacttcctttcttacctacatggatggacatttcttacctgtacacaggcttaaaagaatcaatgtgccatttgtctctccaccctccccaccatgcacacttcagcccatgcttcttctccagcctccccactgaaacttattctaaaagcactgtagctgtagttgtcaaatgcaatgagagactttccactggtttccttctctgtgacttttgacattattctcctgcttcagcctcccgagtagctgggattacaggcacgagccatgacgcctggctaatttttgtaattttagtagaaacagggtttcaccatgttggctgggctggtctcgaactcctgatctcaagtgattggcccacctcggcctctcaaagtactaggattacaagtgtgagccactgtgcccagccgaccgtgccttttttcctaaccccgtaatttttctgattttcctctttggcccctcccctattttcagggtgtctcctcctcactttgcctttatttcttcaggagtctgtttctctctccttctcactgtaacacttggatgaatgatctcatctgctgtgggcctagtttccatctctgcttataaccccaaatctcatctgcagtcgtggacttttctgtctacaggtctgtgtatccaagtcatcatctctgtctgggtgtcctgtagccacctcagactcagtatacactgagtgagtgttcctccctgacacacttcactggccttagccccacacctgccttttctccttttccccatgtttataagtggtgccgttatttacacattttctgaagccagaaacagattcttccctcattcatttcccacgtcactaatactaatttccatttccatgatgtattcctatttctttttcttttttctttttttttttttttcaagacagggtctggctctgtcactcaggctggagtgcagtggcaccgtctcggctcactgcaacctccgcctcccaggctcaggtgatcctcccacctcagcctcctgagtagctgggactacaggtgcatgccaccatgctgagctagttttttagattttttgtagagatggggtttcgtcatgttgcccaggctggtctcaaactcctggactcatgtagtccacctgcctcgacctcctgatgtgttgggattacaggtgtgaggcaccgtgctcagcctccgtaatgtatttcaaatctgttttcttctctgttcacattggcttggttcaggccctactacgcttttcctcgtcattacagtagcctccaaacccatctccttgtttcccttctgttcccacttcagtccattttcatggtatctggagtaatctttgtataatgcaagtgtgttttccactctcctctctaatagtgctgcacaatttttatgaaaaagttaaacttctttagcattatcatactgggctttttggagattgacccctttacacacctctctaaatcttttctacccattttcctagaaattattcatttcagtaaaactgaatcacttggagttcttctagcgtgactggtttttaatacctcagtggcttcaaatatgtcattccctttttgaaatattcttcccctcctctctctgacccatctcaatcaaagttctccttctgcaaataccagctcaacaacctctttcccgtgagtggtattctcatccagtggaggtggtggttctcgttcactaagtccagaggggctcaggaatgtgtattttttaaaagctgggatggtgttgctgtaccttttccaactgatagtcattatgtaatttaaaaatgtgtttctggctgggtgctgtggctcatgcttataatcccaacactttggaaagctgaggtaggaggattgcttaaggccaggagtttgagaccagcctgggcaacatctctacacagaacttttttttaattagctgggacgtggtgatgcatgcctgtagtcccagctatttgggaggctggggcaggaggatcacttgagaccactaattcaaagctgcagtgagttgcaatggcaccagtgcactccagcttgggtgacagagcaagactgtctctaaaaaattttttaaatgcatttctatacatagtacttgacagttgctgatttggttcagaaaacatttgttgaaaaccagctacatgtttgatactatgctatgtatcgaataatttatggtatacttgaggagattaacacataagcatgttacagatattgtaagatatgacagtgcattgatggagctgagaagaggctctgcccaaggaggcagggatggcttttaagaaaaagaatggtagcctgaactaggtaaataaaggggaatggaaaaagaattttttaaaaattcagagtcccagatctttcttttctagatttggattcagccggtctggaaatgtttatttttaaacaatcttaacatgattcttttacactcactatggcaccactcctcagactgacatatggaaactacatttggagttctgaatacgagaaacataaaggaaaaaggatattttaaattaggaagaatttgggctgggtgcagcagctcatgattgtagtcccaacgctttgggaggccgaggagggaggatcgcttgagcccaggaatttgagactacagtgagctgtgattgtgccactgcactccagcctgagcaatgtgagaccttgtctcaaaaaaggaagaaaattgaaaggatttgaagaggtccataatctgctacaacctagagatgcctcagttgtctcagttaactgtgagacattgtagaagagatggacaagaacttcgttgtttgaaaagagagtagcttgtaatggttactttgggaaacgtctttatactattagagactaccaccaaattttgagatcttcctatttgcagtacaggtagaaatcccagtttactatttggtctaaaagggagtgccagcaattgagtcaactgtcatgtcttttcactggccatgaaacaatctgtgtgtgtgtgtgtgtgtgtgtgtgtgtgtgtgtgtgttaaaaacccataccataaatttcttgtaggtgtatattaacagtattgttaactgtatgcacattgttgtacaatagatttctggaacttttccatcttgtataactgaaactctaaatccattgaaaaactccctatcttcctcttcccccagcccctggcagccagcattctgctttctgtttctatgagtttgattactttagacactaaatatatgtgtaatcatgcagtatttgtctttctgcgactggtttatttcatttgacatactgtcttcaaggttcatccatgtgaaacagtcagattttaaaggaaattaacacagagactcatgattctggtagtgtcaggttacatagtccatggtttgttgctctacaaaacaaaaagattctcaaataaacatactttttaaatgagttactagagggggaagaaggaagggaaaacttctatgctttcggcacgctccctgcatgatcttagcaaatggcataactactttgaatgcagatgtcagtatcagctctgagatttgtatgcagctaataaagtagccccaagatgtattaagcaaaaatgaatagaattttggggagaaattgacaaatccatgttcatgggcttcttaataagacaagatatttgtaaagaaatagatttatataatattattcttttttctttttttttttttttttttgagacggagtctcgctctgtcgcccaggccggactgcggactgcagtggcgcaatctcggctcactgcaagctccgcctcccgggttcacgccattctcctgcctcagcctcccgagtagctgggactacaggcgcccgccaccgcgcccggctaattttttgtatttttagtagagacggggtttcaccttgttagccaggatggtctcgatctcctgacctcatgatccacccgcctcggcctcccaaagtgctgggattacaggcgtgagccaccgcgcccggcctatataatattattaaccttaatataagaagaaaaatacatagaaccctatatccagcaattagaaaatgcacatttttctaggactgacatggtctatttgtaaaaaatggaccattgactaggctgtcaagtaaatcttaaaatttaagaatgcctggctgggcgctgtggctcacgcctgtaatcccagcactttgggaggcctaggtgggtggatcacctgaggtcgggagtttgagaccagcctgaccaacatggagaaacctcgtctctactaaaaatacaaagattagccaggcatagtggtgcatgcctgtaatctcagctactcaggaggctgaggcaggagaattgcttgaatccgggaggcagaggttgcggtgagccaagattgtgccactgcactccagcctgggcaacgagagcgaaactctgtctcaaaaaaaaaaaattcctatcttatagctcataattttttatcttaatgcaattaaaatataggttattaataaaaagataaacatttccataaatttggaagttttaacatttctaaatcgtgggtcaagacaaaattataatggaaacttaaaaataaacaatatataaaatactgcttatcaaaacttgtgtaaaggagctaaaacagtactgcaatgaaaatatattgtcttaaatatttatattatattttatttattattttttagatggagtcttgctctgttgcctaggctggagtgcaggggtgtgatctcagctcactgcagcctctgcctctcgagttcaagcaattctcctgcctcagcccctcgagtagctgggagtacaggcacgtgccaccaggcctggctaattttgtgttttttagtagagacagtttcactgtgttggccaggctggtctcaaactcctgaccttaagtgatctgcctgcctcggccttccaaagtgctaggattacaggcatgagccactgtgcctagcccagtattttaagatgaaagttaaaattaatgtgctgtctaacctaatttagagtaaaaataagagaatgagaaaaaaaaagcagaaagaagtaacaacaacaaaggagcagaaatagaaatcacagttacagcacaagggaccaactaaactaaatgttagcctcggtaagactgatcaactctgtcaagatcgagcacgatcaatcaaacaagaaagaaagaaggtgcatataaacaatatcaggagaaagagtgggcacataaatacaggtatacaaagaaagcgtgaattgaatactgccaacaattttatggcagtaaatatgattttttagaaaaaatggaacagattcctagaaaaacataatttaccaaaactgactcaagcagaagtagagtctgaagagtccacttaaagacattaataaacaaaatccacaaagaaaacactgggccagctggttttaccggtatgtttcataataatgcaagtgaatatagatgtagaaatcctaacaaaggattagcaaactgaatatagcaatgtattaaaaagataatacattatgttcacattaagtttatttcaggaatgtaagggtagctaacattagaataccttttttattttttatttatttattttttgagacagggtttcgctcttgttgcttaggctggagtgcggtggcacgatcttggctcactgcaacctccacctcccgggttcaagcaattctcctgcctcagcctcccaagtagtttgtattacaggcgttagccgccatgtctggctaatttttgtattttcagtagagatggggtttcaccatgtttgtcaggctggtctcgagctcctgacctcaggtgatccatctgcctcggcctcccaaagtgctgggattacaggcgtgagcttctgcacccggcctagattattttataattcaccacagtaacaaaggagaaagagccatatgattatctcaataagtgtggaaagattatttaaagtccttgctgaggattaaaaatgacacatagcaagctagaaatagaagggaacttctaagatttctaagagataactgcaacaacatcactcataatgatgacatgatgaaagcattgttttaaaaaaatattgggaacaaggtaagtgtgttcattcttaccacttatacttaacattatacttctgtctccctgaccttatactctaggtcctagttagtacagtaagcaagaatgagaaataaaagccataaggattggaaagaaataaattaactgtcttttaatctgtttatgaggccactatagaactttcatagccaaagcaggaaggttcagtaagaggaataaaagtataaaagttttatctgttgctatataacaaaccaccccaaaacagtggcttaaaacaataggagacatttattatgttcacaaatctaggggttgactaaccccacataggtgattcttgctcaaggtcttttgtgtaattgcatttcaaatgggggctggggctggagtcaccttgaagaagcaacttctttactcttgttgactgtgacctcaactgggctgtcaccaggaacacctacacaaggcatctctatgagacctggactttatcacaaggcaactgagtttcaagagtgagcattcttagagagcaaggcagaagcttcattgccttttgtgacctagccttagaagtcacatacaacttccacaacattccgtttgagaagtaactcactaaggccatgcatgttcaaggagagggtacttaatactcttattagtttgatggaggaagtatcagtgaattgtggacatattttaaaaccatcaccactatagatgatacgaattgcttcgtagataacccctctagaaatgtgcagacaagtcaaagaattattaagaaacattttgatagtcttagaattataagatgatagaccttcaaatcattaaaaagaaacctttaaacttaccagaaagtgcttgtcatctttcttagtaatagcagatttagataaaaacttagttctataataataatccgaataagtatagatttaagttaaattttatggtaactattatgtaaatatttgctaacattttttcttatcccattaaagtttgaaaataaagaccagatgtcagtgtagtgctttaccttctcaaaatccgttgttgacagcttttttactaaaagattttctcactcactgttagggtctttagttctatcttcaattttttttttttttttttaaatattcagttttggcctagtgcagtggcttatgcctgtaatcccagcactttaggaggcagaggtgggtggatcacctgaggtcaacatggtgaaaccccgtctctactaaaaatacaaaattagttgggcgtggtggctggcatctgtaatcccagctacttgggaggctgaggcaggagaattgtttgaacctgggaggtggaggtgacagtgagccgagattgcgccattgcattccagcctgggcaacaagagcgaaaaaaaactccctctcaaaaaaaaaaaaaaaaatctttaattttaatcaaagccaaatcaaagctatttatgatgttaataatttattcctgcgggctattgatgcactggagtggttccaaggaagaagaactagaactcttagagaagaagctagcattgattagacagggaaagagaaaacctaaacagagcagcatgatagctgactttagatacctgaacttttacatggaaaaaagagttctttatcctgtgttaagcccaaaatatagaactagggttcatgagcgaataatagcactggtgaatatcaggttagcattagctcttaagagctgttttaaacctacaaacgaaccctctgaactagtacacttgttattggaggtattcacgcagagggtggatcttctcactgtcagatcttctagagaatatttgtgaatgaattatagagggttgcattagattactttggtctatttcttgtatgcttcatttcttgcccctgttcatctctttctcaccaatttcccgcattcacctttctgtcctattacattgtgaattccttaacagatgaaactttttttttttttggactgacaaaaatcttgtacatagttttgtcatagggtagtaaagagagtattaaagatatcattaagtttctctttctctgtccctgaataattaaatctataaagcacttggggttgggcccacatgtggaaggcatgtgagtaaaggtggtaatgtccttgaaaaagaagcctgtaatattgggttagcattgctgtatccaggtatgaatgcatggttttcagtgagtatagatacacagataaatatgaacataggtatgtgtatatacctatatttcttatccctgtccactgggaaatcttgagagcagcgatagctgatgagccattgggcatgcttagcttttgataccaaattttattctccactaggaggagaatagagctttggagaaatggctggttccacgtctgcgacagggaaagtgtaagaatagcctggaatgtcttgtagtgacagaacataaagacatgcccaaaaaatgatgaagacatatcagaaggatgtagaagccaagctgagtccactggccaaacctgggataatctgagcatgaaaataaataatggttacaacctattgaataaaataggaattaatgtgtctatactgatgtaaagaaacaaatgaatacataaatgagggatagggaaagttctacctttgtataggttgccaactaataaatattgaaatggatactaaagttggtgggtgaaaatttggtgaagaacaaggtatttacataatcttgaagtatccctttacaaaatacttattaaatataagaagaggaatagtacctctacactggagaagtctggccaacaccagccttaaatatgtggtcatgaccagtcatgagatataccatgtcagaatcctgaaccacccgataagatgcagtgaatacttagcatctttctgagatgaacatgccaaaaatatacaaactgaactcatctatagggaaatagatatctgacaactcaaatggaggaacactataaaacaactgacttacagtttcaaaaggttaaaaaataccgtgaaagaacagattgtatccttttgtttaaaggacattattgggacagttggtagaacttgaatggtgtctgggttagtaaagtatcactgttaattttataatttcgatggctatactatgagtaagtaggattatgtcattgtagaagataaaaaaatggaataatcaggtatgtttgcaacttgctgtcatgacttgggaagaaaattctttgtgcttggaatttttctgtaagttttaaaattatttaaacatacaaattaaaaatacatataaacttgaggtactgaatcctagccagtacatgaagtcaagaaaaggaaataaaaggcatacaggttggaagggaagaaataaaaatgaccttattagcagatgacatgtagaagattgcaaagaatctacccaacaatgtcctagaactaataagtgagttaaacgaggttgtgggatacaaggtcagcatacagaagccaattgtattcctgtatattaacaacaaacacgtggaaaccaagattaaaaacacattgttgtttacagttgctctaagaaaatggaatatgttggaataaatctaacaaaatacatataggatctgtatgttgaaagcaccaaacacatggataaaagaaatcaaaagatgacctaaataaatgaaaaaggcctaccatgttcatggattggaagacttagcatagtgaatcagtcctccccaaattgttctatagatctatagtgactcatagtgtcaaattgttcctaaagatctaacacaatttctgtcagaatttcaggaagtttttttgtagataaaaacaagcttattcttttatgtggaaaggcaaaggaactaaaataaaatggctaaaatgatcttggaaaataaaaactagaagaatcactctacctagttttaaaacttaatgcataaccacagtaatcaaaacagtgtggtagtggcagagggataaatacataagtcagtggaacataatctagaaatagacttgtaaaaatatggtgaactgattttttgacaaaggtgcaaagcaatttaaaatgaaatctcacaccttacacaaaagttaactgaagatagatcatagatggatcataatgtaaaagctttcataagaaaatgggagaaaattcaggatgtaaggctagatgaagagttcttagctatgacacctagagcacaattcatttaaaatatcaataatttatactaaatcgaaataaaaaacttctgctcctcaaaacaccctgttaagaggacaaaaagatgagtacagacttggagaaaatgtaaaccacatatctgacaaagaacttagatctggactatatatagaactctcaaaatgtaacagtaaaaagaaacaaaaaccccagttaggaaatgggcaaaagatatgacacagatggcagataagcctatgaacatataaaaaacttcattagccattaggaaaatgcaaattaaaatcatggtggtgggatgtcactagacgcctactaaaacagctaaagtaaagagatcacaataccctaaaaatgcagagaagctggatctcatatatgggtggtgggaatgtaaactggtatagccactctggaaaatagtttggcagtttcttaaaaagctgaacgtatacttagcttatatgacccagcacttgcactcctgggcatttatcccagagaaatgcagtcataatgtccacacaaaagtgtgtgcatgaatattcacagcagctttatctgtagtagccccagattcgaaacaacccacatgtccttcagtgggtaagtggttaactatggtatattcataacacagaatactactcagcaataaaaaggaagaaacttgacgtaacaacatgggtagctctcagaggacttacgctgggtgaaaacaatccagtttccaatgatgacatagtacatgattccatttatataacattcttgaaatgatgagaattctcgagagggagagcagatcggtggttaggggtggaatggggaagagtgtgcctgccaaggggcacacagaacctttgtgctgatggaataggtatgtgttgttttggtcatgtgaatttgtttgtggtaaaatggcatagaactgagcacacacaaatgagtgcctgtagaactggagaaatcaaaataaggtctgtggattgtagcagtgtcttttcatgcttctgagtttgatggccttctggaactaggccctggacagcgcattgctaggagtggggctctgtggtgagtctgacagcgtccctggctgcaaagagcttaactgtggcaagaaggctgctactgggtgcctgggtgctcctgctgccctccaccagcggagctgatcagaatcttgccctaagaggcccctcttaaaagccacagcaccttgggtgggctggtctgccctagagcacttacgctgggtggagggagctgtggaagcgtgcagtgggggagtgctggagtccaggacagtcagctgtactcgggattggggtgagggtgtggttgtttctagattaaaatacagatacatgatcaagtagaattctgaaattcctttaaatgtttaaggtaaaataggaattaccaaggaattttgtactttaataccctcttagggtattaaggagggtttgtcagtgaaaataatcaagaaggactggtccattcgagccactgattttgacagacaagaaaacagacccctgagtgccccttatcataggactgtcggcgactgggatgctgatagtgtttctcaagtagtagtcacccttttctgggaaatcatgactacgtctggaatcatgtaggttggtagagccaaaagagagatcagaatccacatctttagatccctcaaactcccctcacccatgtttttcatctgcgtcgcaaaattccaggtacctgtactaagaagtcctgaaggtttctgcgcttttcctttctgccgaattagtttacgcttaagaaagtcagttaacgtcatttcatttcctgcttatggttcagaatgggttgatagtgatttgtcattctaacaaaattctagcaggtgtaggaatttgaatatgtgaaaatcttctggaattcatcatataaacaatgtttgaatacttgctgacttaaattgttatgacactctactaggtaggcccgctgagaaaatggaagtagcagtcctttttccctgacagtcaaatttaggagaaatgaatcagtacaggatttcttgactctccgaacctaaactgcagactgctcctctccgctttgtccccgtggctccagccacattcttcatgtccctcagacatgttaggtatgtaccaccttaagtcattcgccgttctctgtgcttggaacactgttaactaaaatatccgtgagactgaccccttccacatctgtgcttacatgtgacccacctgggtggtcagccacaaggcccatcctgaccaccctgtttagaattgcaacctgcccctctccccagcgctgtgagtcccgttactcttttttcttagcatttattactcatatactttacttagttatgcttattgctatatcaccaatatagatatttggtccgcattgtttgcatggtgtctcatgggcctttggatgtgatgaaagttaaatcttttcccctaagtccctccaccttcaatttgtttaggctctaactctcctagaagcggagagtctgagtttcaaagaaaatggtgcaatactgctgagcgccgtggctcactcctgtaatcccaacactttgggaggccgaggcaggtggatcacctgaggtcgagagttcaagaccagcctgaccaacatggagaaaccccgtctctgctaaaaatacaaaattagctgggcgtggtggcacatgcctgtaatcccagctactcaggaggctgaggcaggagaattgcttgaacccaggaggtggaggttgtggtgaactgagatcgcaccattgcactccagcctgggcaacaagagcgaaactccatctcaaaaaaaaaaaaaaatggtgagatacttactgattattttaaagtgaagattagtatgtcttattttatacaagctaccaaagaggaaaaatgtctgttttccaaactaatgaattttacctattttgtaaagacatttctcaggtattcttttcattgcagactcttggggacagattaaagaggactttttcaattaggagatttgtggaatcagaatttaattagtgacattgtcctgtcaagtttaggatcttgaaatcatgatgggttgaaagacgctgatgatttcaaggacagtaaactattgtttggaaaaggttgaacttaccaaatgtttcctaaacaatgtatactttattagcaaagctgtgatggtgctaccttggaaatactctttacattcccaccgccactacttgattcaggcctcatcaccttttgattacgctttcagtgtattggtgtatgaagttgccttcttcttggttcagtccatcttctatgctgctgagagttattttcatgaaaatcaaatgtgtttataccagtcctttaaagaacttcactggggccctgtccagagcctgtgcctgtagcacggcccacgtgcccttcagggtccgcccttaatgtgtctggggagccctctctagccagctcgccagcctttctgaatctgccagtcctgccgctacaccattccctttcattcctctttatgatgtgccctctgctggaatttccccttcctgctgatctcactaggcctattcaggtagcaactactcaagctccttcccacaatattttatgcaataaagtatcagtactcagagtcacggcttaatataatttcttttttcctttttaaaagcttcaaacaaataatgtcttacctttagatccctataggttaaaatatgtttgttgactaaaaattgtgtatgattgctgaaagtattcaatgcagggaacagaccagttcatcatggaggcattccatcagagcgtctagttagagaagatatgtcatggactgcatcggcacagaagtggggtttatgtgagagaggagttggaagtcacacctgagtggagagcaacgtgaaaaggtgatgtcagcaagaatttaggatgtatggaaaggatggtaaaggcaccaactggatggatcagggagacatggaatgcagaatgcaggaaataggtaatctcttaaattgcacaaatctaggtttcatttttgtgctctagtgtgttacacttgtgatgacttgacttccctgagtgacagtgcgatgggtgtgatccatattcatatgcacttcatagggctgtgaggactgagagaggtgacccagtgacgagtgtcacacaaacctgacatgtcgtaggcaccaaatggttctagacagtggttctggttggttttgtggtcaattggaaacctttcatcaagtagaattttctttgcagagcaaacaattgaaatgttaatattctatgtgtactaaagtttgtattctccattaatatgggggagtatatggtgacctggtgtttagtgccagctttgtttttggttcattgctcgatttatgagagagaatgcataatatttgttgttcgactattttacttgcaggattgaagagagttgacaaatctagaaaatgctttgagttcttggacatcactgtttgaaaacgtactgattacccttagttgagaatgggaaaatctaaaggggaaaagtcactggttcacgctgtactattagaattggtcatcttcttgcctgacttgtaagttaaggatgcttgatctttgctgtgatttaacctgttcaggatgaattgtaaccagttacaatgaatacaaattctcctttctgtttgacctggaaaactttccaaaaggagatgtctttacaataatgatatatttggtgaaagctgtgggttgtttttttttttttaaagcttatgatgacagtattgaaagaggaaactaatctgtattgtcagcacagccacagctagcactagtcatttcttgagaggtggagctgtttctcagaaaacatgctgtttgagggtaggaggtgaagggactccttgtcagaaattggcttaattttaatccaagttataaaccacagttctgtttgggtattgcatttcttcatttactcagtgacaagaattaaggcagatgatgcctgtagaaaaaaaagtgtatgttcttgatagttctgctactagtgctagtagaggttgagaaataagatttcctgtgaagtgtggtttggttgcttttgtggtatcccatgcaaaagagtttgaactatcttcatttttctccctttactggatcctttaaaagaggaaagggtgagataaccagacttacaaggatggtaaagtatagtgggctttggggcttacttatacagcccaaaggttatgggatgctgctgttatgataatctcatgtcagaggttcctatggaagaggagaaaggatgtacctttctgtgtgttctcattcagaagtcaaaagtttgttttttaatgattagtttacagatttggggctgactgatagtatctgggcagggaaaagagacctgtgactcaggttgttaagggaacctgtgtatatgctctcacaaactcgaaaccgtgtgttgtgatccttaattggagaagaggtagtgcccttctttgttatcatcactaaagagtataaattataaagtcacagtttctcataaagaggctttgatagtttcacttcctgaaatgtgtatattcacaatcttttgcaatagttattttaaggtgatgtgttttatttttttctctactgcctcttcgaaatcaaaaagttatatatactttttattttatattaatctttttcaatttttgtttttataattttaatgtattttaaatttattttaaagttttatttctcctttttgctttaatatttcttcctttttaaattgtttttcatttttcctttgtatgtgtttttcttaaaagaacagaagaattaaaataatatctagtgatgtaggtatttcagttttgtttttaatcagccttgagaagaaaatacaggctcaaataacatttgtagtcagttctaaatagctatagagttttcccaaggattacctttcttttttagtattatctgataacaaaataaaaaaaaattttcctgagaaaattcaccgttcctttgcttttgttgactctgtatattaatgttaaacaggaagcgaggccactgccaggggctgagccctgcagacccagtgccctgcaggtagtgccctcctccctactgcctcccaggactggatctgggcttttctggtgtgtggttactgtgcttatagagcattgaggtaacacctcgtgcatattttacagatagaaggaaaatagcagtgaattttagttcagagtcctgagggtgaggtatcaaatactctggctgaaggtctttctctaggtttacttcctgcatagctgggcaagggtgtggggtggtgccatgtggggtaaccagattcccagcaccctgtgaggtggaccttcccgggttttatggattcctaccgcatgttagctaaatggctgttttcagttgtgcaatagatatgagtagcctttggttagtgttctttctgtgattacaacatgaggaaatacattcctttggtgtgatattttagtggaactttaattttaaaatcatgtagttttctaaaaccaggctttctttttttggtatgttgtctaggagatatgtggtctttattttaattaagttgctgacctttttactattttacataatataacagttaaagggtgattaaactgttttttaaaaaagtgatttttaaaaaaaagtgcttgttgacatcatctttctgagaacactagtatatgtgatttactataacaagatcttagtttttcctgttggcaaatgacatgtaatatttacagaggtcttgggttcagtccagcagaaacatctcagataaattgatgtgtgaaactaaagccattttacattttctttcatatggagataatataaacagttttttcatttaaaagtaaggagagactggctatttagtatggatttctttgagagggacaacttgtaatagtatgcattctctagagttaaacatcaaacaccatgtttttagttttataattcagtgatatgcatccggcagttgtgtgttgaggattctgagttaattcatgggctttttctcctggtgtcatgcttctcttgctttcccatctaaatgtataaggtcacattttgtatttcacatatttaaattagcataaattctcaatctatgagatttttacttttaaaaactagaaatatctctaaactgttaacagagtctaagtagtgaagtatcttcagtggtcttttaaagaatacagttggaatattccaactgtctttaaatattttcagcatttaacctaaacacagttcagtgctaaaatttatttaaatatagtggaattcttatctgttgagaaatgaagaggttgaaatatagtaacctcggaaatctagtctcatctatttgagaccagttatcgttttgtaggtagaccaatcaattcttaggtaccaagaatctgaatgatccttttacttttaggaaggtaagtactttattcatttaactatgtttactcctggtcgatttttcaagtcacaatggctaatgtgctacaaacagaagtgcttcctttccagcactatagtaataaacaagattgcattttatactccttacaaaaaaagtagagaatagtttaggatttgtctcaactctattatgatgctaattcatttttcttatttcttctgtctttttataaatacctagaatattaatatgaagataaaaacagtaattttgaaatgacagttgtggtttatcattctctattttcagatgatcacagtcgtgttaaactgcaaaatgctgagaatgattatattaatgccagtttagttgacatagaagaggcacaaaggagttacatcttaacacaggcaagtaatacgataccacgcaaatgtctgaaaatgatgtgtttgtgcttgttctgctttaaatcataggtaaaagtatatcttgacttcttttggaaatgaaatggtatttcagtttttttctgacgatgtaatgaattatggacattataaggttttgaagctttgagtatttaagataaaaggcaagttatttttgatattacacgttctgtgaaggaaaattcttaggaaatggcttaggccagttctttggcagattgtgttccttacattatatctgacacagagtgctgcttatgcttcttagtgtcttcttttctcttcccataccctgtggcaaaaccagaggccctggacagctcttctgtagctccccctgcctcgcatatatcttcagagagtacaccaagccctggatggtgtctggtcgtcttcaaggacctgagcgggtgctgagaccaggtgctccagaggcccagccacaaggccttgtgctccctgtggccttctgcccagttgcctctgagcgcatcagccacacctgtgtgcaagcatgattcttccttccattctgagatagtaggagagatttttgtgtatccacagctactctttattcatcaagtaataaagacttgattatcaacagggctgtctttccccctaaagatttttaaatattcaaaagatataccataatatttgagatttttgaaaacataacttaaatcagaatgacttatttgaaagagaaaaatatttgataagtaaaaagatatgcagattaatgtaactatgagctattatactacgtcaattcacaggaaactaaaatgcattttttacttgaggtagtacagctcctgcaagtcaaaaagaaaaaaaacagcccatggaaaaaactggcaaaggacataaactcctagttcaaagaatcaaaggtggacactttgcacatgaagaaatgctccattcagtaaccagggaatgggcctttcacctgtcagaaaaggagagatttcaaagtgtataatactcagtgttcaaatactctgaccgaccaagaaagtctacttagagaaatacagtcagacataaagatatatgtgatcggccaggcgcggaggctcacacctgtaatcccagcactttgggaggctgaggcagatggatcacctgagatcaggagtttgagaccagcctggccaacacggtgaaaccccatctctactaaaaatacaaaaattagctgggcatggtggcgggtacctataatcccagctacccaggagtctgaggcaggagaatcgcttgaacccgggaggtggaggtggcagtgaggcaagatagtgccactgcactccagcctgagcgaaagagcaaaactgtccaaaaaaaaaaaaaagacatatgtgatcatgttcattgcagtcttattttaacgcatcagactcaagcaatctgtaagggactggttaaataagttaatcatacatataccattagtaataatttgtggagcttgggaatagagactttaattttccattttattcctttctgtaatattggaagttttttttatgtgcatttgtattttaaactactatttagagttaaaaattttattttctgtttgaaatacctatagttgacccttgaaccatccagggttgaactgtactagtccacttacaggcaaatttttttcaaccaaaaacgggtcggaaatacggtattcatgggatgtgaaatccatgcatacagagggctgactctttgtatctgcaggttctacagggccaccagtggggcttggacatgtgtggattttggtatactgcagtgggtggggggcaggttgggtcctggagccagtcccttgtgtatattgagggacaactaattacattaaaaaatagagaagaattaccaccctagatcccttatcacaaccctttctccctctgtactgttcttctccattatcacttactggttttttgttctcttcttagttcttaaaaacataggtcttaagattctaaattagcttttttttttctcctccagtctgcactacagccctttaagaccatggttttcaacctttgtaacctaagatagggtagactactgtcatggtacctgtgctggaatcttggtgccagcatttcactgggtattcactcacccagcaaagacttactgagacctactctgtggcaggcattgtttaaataaaacagactttcataaaaaccctattatgaattttatgaattttacattataataggaaataataacagttaaaaattttgtaatggtgtttttacatgctttgtataatttaaaccttaggacaaccctctgtggtaaatagtattacctttattttatacacgaggaatctaaggagatccagagacattaaataactttctttttttttttttttgagatggagtctcactctgttgcccagcctggagtgcagtgatgcaatctcagctcactacaacctcctcctcccgggttcaagtgattctcgtgcttcagcctcccaagtagctgggattacaggtgctcaccaccactcccagctaatttctgtatttttagtagagacaaggtttcaccatgttgctcaggctggtctcaaactcctggcctcagatgatccacctgtcttggcctcccaaagtgctgggattacaggggtgagccaccgtgcccagcctcctagtaattgtttttgaacttcttgtcgctgtgtgtgtcctgaaggagccctgcccatttaatgcgagaactcagaagcctgtgtctggctttttcttctctcctgaattcctctctatgtttttcaggtctctattggatatttttactggatttcaaagtctgcatgttctctgtcttttttaccaaggtagctgccttcctatttttctcattgggattgagactgtcctcattgtctgccaagttctttctgatcttctattgcccctcgcttttttcagagctctcttcaattacacctgtcctgttgcagcagctttttaaggtctccccttggcaccatccctgtcactctagtggtgtagtcaccaactacatccctccacttctacctcactgatctatttgtcttttcccaaatatgccctaaactttcatgcactttgccttatgcttggagggcctggagtggaccctaactccctcccattcttctgcattccctctctgtattaggacctttttcgaggctcttctgtagtttccttcccagcctttcctgctccctaagacctgatggtttaatttgcccatactcttgtactacttaactgtgtccattagtcatacaagtacttctccttctacctttgcaagctcttaaagagccaagactgcatctcagtctagctcttggccttgcatctagcaggttgtttggaatggaggatacagaaaactagagatgcagtaacccagctcttctgattgtcctttgtagattgttacttcagtcatctttttcttctgagttttatgaattagtatttttttaaaagtcacctccttggtgtattatgtctgttttgcagatgaagaacattggtagcattgctggtaaatggaagagctgagatgtgaatctctgttcctctgatttaaaaacctatggctgggcgtagtggctcaacgcctgtaatcccagcacttggggaggctgaggcaggcagatcacctgaggtcaggagtttgagaccagcctggccaacatggtaaaaccccgtctctactaaaaatacaaaaatcagcctggcatggtggtgcacgcctgtaatcccaactactcaggaggctgagggagggagaattgcttgaatccaggaggcagaggttgcagtgagccgaggtcgtgccattgcactccagcctgggcaacagagcaagactccagctgtgttcctgtttattatttagtctttagaatcaccagtggaatggcatgggttgtgattgtgtgtatacactaaggagtaacctcactggcaaattgctacatccattttttttttcctaattaaagaaaaaggtattcattttactggggatttcctcattgctgtttttacctttagactgtgttttgttttagaagtagatcaagtaatagaaaaataagttgagtgaggaaaacgccactctcaaatattaggttgtagtttgtcaggatcctcagtagacttctagcatttttacaacttaaccttggtttattcatttaagtaatatttattgagtatctgctacataccaaggagtacaccaggcctaaagatgcttttagtcagctgggcaccatggctgatacctgtaatcccagcactttgggaggctgaggcaggtggatcacttgaggtcaggagttcgagaccaacctggccaacatggtgaaaccctgtctctactaaaaatccaaaatttagccaggtgtggtggcgggtgcctataattccagctccgtgggaggcttaggcaggagaatctcttgaacctgggaagtggaggttgcagtgagctgagatcgtgacactgcactctagcccgggtgacagagacaccatctcaaaaaataaaaaatgcttttagtctagaaccactttaacccccttgttgtatgtgggtctgaagctctgggcagccctagcccccactcctgactctgcccctaactcataccactctgctacattccatgtgagttgaatgcatgttcatagttattttaggtgtagcaaaactacatctcattttaaccttctcatttaaaactcgttaaaatttgtacataggcttttcatctcttattagatcttccttttgccctatgctatcattaaaatgtgaaagcaattggaaatacatgaaatattttgcttttatccccatggcattgttgtaacatggactgttgcccctttttaactctgcccaatactcttccattgaggccgtacatactgtgcatttgtagattaacagtttcattgtggttcaggtactttaatgattcctactttttctagaaatctttttgtagttgacttgagttggttggctggccagtgatggtacagacttttttctcatggattttctatgatgtgtgggaggcacatcctcttggctgtagaagatgaagaggacagaaatttccttggtctaccttctttgcagctagaacatgatcagtcagatactcctactcgtgattttgtctctgatttgcctatagattgtacatgaatactatagccaccttcatatggcagtgccagcaaaagcaaggagatagtagtgacggtatcaagagcagcatcccaggttgactttctttggggcaggaccttgcttgtgtcttaacccttggttcctacccaagtttgtctctctagtcctcaactctgaaagccacttgatatcttacacaattccttttttgcctgagaaaataaaagtcagttttgatcatttacaaccaaaaatcctaactaacacagacctgtttttgaaatcaggtagattggaagtcttggtttacttcttataagcccgtccgctgtctgtcttgtaacatcccaggagcagacttaacaaggccttcctggagccttgctctttctgtctgcttccctcatctgctctctctcctctctcttcacagggtccacttcctaacacatgctgccatttctggcttatggtttggcagcagaagaccaaagcagttgtcatgctgaaccgcattgtggagaaagaatcggtgagtaatatttaatatttacaacttagtatactgtatgtgatcattagcatataaagattttcattttggggccaatattcattccttagttttggcctttaattgctaaaggctagcggtcaaagtgtttctgtccggaaaaactcatgtatccttttcctttcttaagtatttttataacaattgcagacctttcatctccaagatagaaatacaatggaataaatatggactagcagtgacatatagatctttggaatccctaggaatccttattgacctattgtcaataagtcctcttccagctttaagatcttgtactgttctacttcagatttcttactttattctacatttcataattgctggtttttaatgatgatgaaaatttcctaatccacttctaattagatgtggcaatgacatgccagaatttccagcagtatcagcatagtcatttacaggggtttttcctgatcttcaatatcatttaaaaattaaggattaggccaggcacggtgcctcaaacctgtaatcccagcactttgggaggccgaggcaggcagatcacttgaggtcaggagtttgagaccagcctggccaacatggtgaaaccccgtctctactaaaaatacaaaattagccaggtgtaatgctagctacttgggaggctgaggcaggagaaccgcttgaacctaggaggtggaggttgcagtgagccgagattgtgccactgcacctcaacctggatgatagaatgagactccatctcaaaacccactgcactgcagcctgggcggtagagtgagactccatctcaaaaaagaaaaaaaaaaaaatcaaggattatgacattgtcagtttgagcaccattgtagactacatgatttgcaaattttatttctctcattttggagtataattactgtattaataagataacactgccaccagaatttagaaatcatgtttatggtggttgaacctagatggttaacaagagtcaggtttaattattttggggaagattacttcagaggtggtgggtaatgtgttattcatcaagagacccataatgtctgattattgttctttttatttcattaatagccattattggtcagtgttcagcttcttaattcattatgggttacaaatgatgataatccaattctgtcattacttctcttaaaagcagttttccaaaaaaaagttgattccctattgaggtcatctgttctcaacagatgaccaattaggagtttttctggtggagagtggcaggagaggttgaattattatgggtttggacatactggattaattttactcctttgcagttattttccttatttttgatcaaattaccctgtcattggctagcagcaatgtctttaggttgggtcctgagtcctctttttttttttttttttttggagacagagtctcgcctgttgcccaggctggagtgcagtggcatgatttcagctcactgcagcctccacttcccgggttcaagagattctcccgtctcagcctcccgagcaactgggattacaagcacccaccaccatgcccagctagtttttgtagagatggttttaccatgttggccaggctggtctcaagttcctgacctcaagtgatacgcccacctcggcctcccaaagtgctgggattacaggcgtgagccacctcacccagctctgagtcctttttaacaaacctttaatagtttccttggctgggtgcagtggctcacacctgtaatctcagcactttggggagctgagttgggtggatcgcttgagcccaggagtttgagaccagcctgggcaacatggtgaaactctgtctctacaaaaaatacaaaaattagctgggtgtggtggcgcatgcctgtagtcccagctacctggggtactgaggcaggaggatcacttgaggccaggaagtcaaggctacagttagctatggtcgtgccactgcactccagcctggggacagagcgagaccctgtctcaaaaaaaaaaaattttttttgctgtctgtaatgactgcatctcaggctcatctcaaactcttgctgcccagacctggaaccagccatttctctaaggagttttggtttcttttaatgggcagcagtatttcaagatcacagcaaaaataaggatatatattgcaactggactggtcattgtttttagggcttttcagtggacaaaagctaaggaacaatatgcatatgttaagataacatacttcaagagttcataatgatattcccagttcaaatttggaactataagtgttgcttagccccttccatcttacatctgctatttcctttttcataccaagaatcctggttctcaaagatatgagagatgtaaactatcttgtaatgactcatttccttcttcccatatcatatacaatatataaccataatcatgatatcatctgctaccaaaataattactgaaaatggtttacaatttttaatatttttttcattctcttcctggtgtttctgagttgtactttacaaactgtagtctctcccttacatctcttgtagactcttagttctacaagcaagcattatttaatattcatcaacagtctttgctgatgtctctccagtcattctgactaaagctcattctctagtggctcctaaggagggacttgtgagcattatagtctgatttttttacatgtcagtgatggttcatctgctgtcttaatatctgaaagtcagtttggcgggatataaaatccttggctcatgtgctattctttgaacaactttttttttccatttccttctggtattccaccaaagtctaatggacatcttatttcctttataagtctcttgttctttttatctgcactacccaaagaattctttttttctttttcttcagatattattagttttaccaaaaatatgtcttagtaattggccattctgggtcagtttttacaggtacatgatgtgttcttgagagtttgacttcaagtactttttaaaattttttaaatactttttgttaaagaaatatttgttgaattacagtccttagtatttgttctgctcccttgcttgctttaatgttcttctttgggtcttcctattatacgtatgttggattttctttgcctaccttttttatttgtcacgttctctttaatcctgtttatctctttatttctattttacttaaaaaaaatttttttttttctggccaggttggagggcttacacctataatcccagcactttgggaggctgaggcaggagggtcacttgagcccaggaattcaaaaccagcctcgacaacaaagtgagaccctgcctctacaaaaaatacaaaaagtagccaggtgtggtggcacgcacctggagtcccagctggggttgggggattgcttgagcccagaagattgaggctgaagtgagccaagttcaccccagtgcactccagcctgggctactacagagcaagacactgtctcttaaaaaaaaaaaaaattatttattttcctcttaaggcattattattattattatttttttttttttttgagacggagtctcgctctgtcgcccaggctggagtgcagtgctgctatctcgactcactgcaagctccgcctcccaggttcacgccattcttctgcctcagcctcccaaatagctgaggctacaggcgtccaccaccacacccggctaatttgttgtatttttagtagagacggggtttcactgtgttagccaggatggtctcgatctcctgacctcatgatccgcctgcctcagcctcccaaagtgctgggattacaggcgtgagccaccgcgcttggccagcaacatttattttgaagataaaattattgcaagagtttgataattttagaaacataaagtgaatgtcccatgacgttaaaatataggtaatcatgctttaagaatgataaatttatttttattttatgaatatgatacctatggcttttttagtttataagttacaggaacacttaaaaaataccttataagaccggacatggtggctcacgcctgtaatcccagcactttgggaggctgaggttggtggttcacttgaggtcaggagttcaagaccagcctggccaacatggcaaaaccctgtctctactaaaaatacaaaaattagctgggtgtggtggcagacgcctgtaatcccagctgctcagaaggctgaggcaggagaattgcttgaacccaggaggcagaggttgcagtgagccaacatcacgccactgcattccagcctgggcgacagagcaagactccagctcaaaagaaaagaaaagcctatggagacagaaaatggatcagtgcttgcctgtggcttggagtgggaataggacacaaggggatttctgagtctgagagaaatgttctaaaactggcttacagtgatgactgcacacacataaaaaatgctttataaaatttcaatctctacccgccctgctccccgagatggagtcttgctctgtcacccagactggagtgcagtggaatgatcttggctcactgcaacctccacctcctgggttcaagcgattctcctgcctcactctcacaagtagctgggattacaggcgcccgccatcgcgcctagctagtttttgtatttttagtagagacagggtttcaccatattagccaggttggtctcgaactcctgatctcaggtgatcctcccgcctcagcctcccaaattgttgggattacaggcgtgagtcaccgcgcccggctataaaatttcaatcttactaaaatacattaagcgtagaatatagtagtgacttcttagttttgggtacttgaaaaaatatagcatgtatatatctggtttgttttcattttttaactttatggtttcatgatgtctataaactaaaaaaaaatcatatacatatttctaaatttaggttaaatgtgcacagtactggccaacagatgaccaagagatgctgtttaaagaaacaggattcagtgtgaagctcttgtcagaagatgtgaagtcgtattatacagtacatctactacaattagaaaatatcaatgtaagacctttcttgcccgaaattgtggactttatatgatggtttaaagaattattctttattattagatttgaagtttgataagcacatgcatttggataattcctatagtatctagccataatcacgtgtttttagattgtgtgtctgcatctagtagctttctgaggactctgaagaaacagtagaaactaagaatttcaaagttcagtttgatatttggggattaaaaattaattaatagaaatacatgaggattaatagagagggctttagaatcaggcaaacctggattcagatccagctctgctgcttagtagagctgtactttagacttgttccccaccctcactgatcccttcctcatctgtaaaatggaggttaccacgccttccctccggggctgtcctgaaggtcaaatgaggatgtgtatgcagagcacctgaccttagtaaatgcccagcaagcaatagctcccaggacctgtgactgccaagaatagcaaaatagtcaagaaaataacttacaaagctattgttaagtgattcacaacacaaaaatttgtccctattgagttggaaaaaaaatggtgttttatatatatatgtaacacgcacacatacatacatacatacacacacatgtataaatgtggcatgtggtctcaagaacatttacttgggtgtttgtttggttggttggttggttggttgattggttttttgagacagtcttgctttgccacccaggctggagtacagtggcataatcacagctctcaatgaggcctccaactcctggcctcaagaaatcctcccacttcagcctcctgagtatctgggattacaggcatgagccaccatacctaattttcttatttttttttaatagagatggggtcttgccttgttatccaggctggtctcaaactcctgagctcaagcgatcctcccaccttggcctcccaaagtgttgaggttacagtcatgagccaccatgccaggccttacttgcatttttcttccccagaaacatatgaactgttccttgggccttggttgaggtacatacacatagtgggttatgaacctgctgttgccagggtgatgattgaggcatttctccccctttcgccatttacctcttttgccctctttgtgttgtaatagaataaactagaaaataatgaaatgagagtaattctgagatgctaatagaaactgtgatagcttgaaaataggagaatgtttagcctcatatgactttaggaatgtatgtagtctataagtgaaatactgttttagttaaccattaagtaaattaagtgatcactaacaatacaaaaaaaccttaaagagaagggagatatatacatgctttttagatgtgtggcagtcccttttatggtggatcccttgaaacacttgtgttttttgctgcattgagtaagcacaggtgcatgctctgtgcgtgaggcctaggttctggttttccttctgtccagcttcccgtgacatccgtactaagcttattatacagtctcttgagtcacctaaaaaggctcaaaattacgaatgataatgcttagatttatttatacattcccattactatatttattttgtcccaactctttccatttacatgtgtcttaaactgcattgagatacatcaataactctttgatactggactgagctgtacattgttcattcccagaaacagaactgtatttccacaaggttctgaaagttctctgaatcatcttctcttggtatttatcccaggtaattactcatttgcttatggcattaaaattgatttagatgaagggcatagatgtgtttccctgtttctgaaacagtccttattgagaattgattataccttaagtaacaataaaatagaatgatttttgtgttttgtttatcagattccaaaatgctttcacatatatatcatctgaccagtagtcttcagttagtgtagatagtgtaaatattcttattatccccgtattttatattataaaaaataatagttaaaatccattaggttataggaatttttccatcgattacttagctaataagtgacagactgatctcattagttctcctggtcccctattttttaagacaaagtctcattctgttgcccaggctggagtgcagtggcatgatcttggctcattgcagcctccacctcctggattcaagtgattctcctgtcctagcctcccgagtagctgggaatacaggtgtgcaccaccacgcctggctaattttttgtatttttagtagagatggggtttcgctgtgttggccagactgatcttgaactcctgacttcaggtgatctgcccgcctcagcctcccaaagtgctgggattacaggtgtgagccaacacgcccggccatctcctaacttttgacatggggataagtccttttgaaaagatgctaattgttgtaaagagagattattgattctgaagcatctttctggagttgaggccatcatgtgagttggccagccactctgttttagttgctagcaactgtgtggtataatgacatgatcactggattttttattggcctgggtttagaacataccacaatataatagcaagaatttgtttgtgttctactttataatttttaattagaactagggattctttattctttagaaggataaagtagcatttcagtatgagaggcagaattacatttgcctaagtagataacctctagaatcagatagcccaagtttaaatcctgactctgccattcttgctgagtgacttgggtcacattactttactatagatgataaacagtacctgttttatcatctataaaatgagggtgataacgtacctacttcataggattgctctaaggatcaagtgagttaatatcatgatggtgcgggagccacaattacctttttagttgatcagtttcctttttgtaacagtaatgaattggaagggacaacaggaaccgggagtagttgaaaaggatactagcaagaaagaaagaaccaatgggagtgagaaaaagtgatgatggagtagggggctgatagccaatctcaacacgcagatctctgtctttattggagtcgctggggtgcagagatccagtttgcatctaagggagtctgacacgtatctattaacaagtccaaaggtaaagctgttgtctaagcaagccctgcgcctcctccagtctaatggaacagcaaaatgacataccaggagccctttagtgacttacttaaagctaaaagtttgaactcttgcccctgaagaagagattgcatctcggctttcagtgtggcctgtggcttaagctatggagataattttgagggattttaaaaatataaggtcatctagagtacttgtctttcagttttttctttttagttgcccttcactatttctgtaatgttggcccgctttacgtttagtgagttccaggaatactgttataatgctgactgtcttctatttccctgtccctaaagatttagcaagatgagagtctctaattattatgatgcttagctgggatttggttcaaaatagtttaaacagtttattctacctagaatgtcctctctcttctctgcacgtccttagtaacccctgtggcccacttcttacttaggtctctcctaacatgtatctatgacacattgatccctaacagctatgattcttcttatactttttcagtaatttaaattttatcattctactgcttgttcaatacatctctctatgtaaatcttgactccataatgaggtttttaacttcgaaggggttggaagttatctgctgccttggtacccccccgccattacacaagagtacattttaagcacattacacctgagtgattgttgtaaaacacagatgcaatctttccaccatcctctaagaattcttctgtggcttccattggttaccaaaaaaagtctgtgtccagtgcacaagaccatgacccagctcagcctggtccttgcctccttgtctaactccctctccttcgggtatccaggatttgttcgtgctaacagacagacttctctaataggtgaattccttggtgggggtgggtggcatgcctttgcatattgctttctctctgctgagaatgcaagcctgtccctagccgtttctgccactcctcctgatctggtgagtttgatctgcctttcacaatgaatcccagtcagtaaatatttatcactgacatgctgaccctggcaaaaattgatgagagactcaactaaggcagtgatagtgaaggtacagatttgacatctggttaggacatagggttgacaaactttgtcactctttaggtgtagaggaaatacccaatgaagaggttgaaaatgtgttgagttttacaattgactggagctttctaggggccacaataaaggcaattggtgagtatagttgttttaaaaatatccagtatctgttaagcatttaccatgtgcagggatatggcaggcacttcacattaattgttttatttattccttaacaacagttttgtaaggtaagtagtattattaaccctatttacagatgacagaactggggcttagtgaaagtcttttagaaacaaaatcttggtaccttccatatatacctaaagtgtgaaagtaattgggcctggggtcatagagctgtatagattttttatgataattggaaaatgtataatcttattatatatttatgatgattattaacttgcatttgaaggctgtgaacagggcatatgcttaaattgctgtggcctaacaattcctgttgttacacttgctcaactcacaggttattttcctggcttcatatatgcaagtttgacattgctgttcaaagactttaaatttagaattctcttttaaaaatgtaaatgggattttgccttagaacagcaagccactgacattttgtgcagaatgtagtatacataatgcagagttttacacagaaaacttggcaggcatgtgtggaacggctgcagacagggacagcctataattgtgggtgcaacccgcgtgtgtgtagggaggggggtgaagcagcgagaagggtggagggcagcagctggggaccagaggagaagtagtgtgactcggtgaccaaggagaagaaaagggtgcttccacagttttgaaaataaggagtttgccaaaaattgtaaagtctaaatgcagaccttgtttggaataaagatgaatttcgttttagctctgtatcctttggcaaccctttttttcccctctcattttttttcctgcgtgaaaaagagggaaaggggtccatccagtcatgcatatgaatgtttctataccagagtagtaaatatatgcatcttatgccttcgtgtggtcctttgtaactgtgaattatgattttggaaagctgtgtttttttaatcttgtttctgtaaggataaattaatcaaagttaacctctcaaaaaagtcctggttctgtaggggggaaaaaaaacaacaactgattttcaaaaatctattagtggggaagactatattatacaaatagtcatgagcatttctgggaactggcttccaatatcccagacagcgtgacctgaatgtgaaaaaagttgagaccactagttttgagccaaaatcatacaagcaaagagaaagcaagctgttgcccaagattgtggggtaaccatatgtttgagccaggacttgatgaagtaattgtgcgcaggaccccgtaggggtactgggccgtagcccacagagcactgcaggcccctgctcaaagcagaccacatgaaggcccacacagactgagtctggtcctgctggtggccgcagcaagtaagaaacaaagtgggttattaacatccactggaatcttaacctcatttaaaggaaaacttgactttgagttaactgtatcccccccaccccccgctgtcaagaacccacctctctagctcattcctgtgtcagtgttgccgtattaactgcccacctgctgctttgtaaaaggagactagagtttacccttcttattttagaatgttttgaattctcttttttaatgcaataaatttaatttaccttttttttttaacctacaataagattgattccttttggtgtacagtcgtgttgtgacttttgacagtgcattcagtcatgtaaccactgcccataacagaaacatagatcggttctgtcacctctcccctgccccagatgttcttgtgttgcttgtgtacagtcagtcctgtcctctacccccagccagaaaccagtaatctggtttctgtttctgtttttctgccttttccagaatgtcataaaaatggaatagtatagtatatagcctttagtctggctttgttcacttaccataattaatttaaaaataaatgtcttaaagcgtagaaaccatgatcattttaatttgtaatggagaaattttttattgcttttagtatattgagaacttgtggatcacactgtatttgaaattattaatgctggatgttaaaaggtcactaaaagtatgtactttttagaaaatttatttcactgtattttattagtattataccttatatactgagtctgggacatagaagtcttaatttctactgcaaaatggatttttttttttttttttgagatggagtttcgctctcgttgtccaggctggagtacaatggcacgatctcggctcaccacaacctccgcctcccgggttcaagcgattctcccacctcagcctcccaagtagctgggattacaggcatgcacctctatgcccagataaggtttgtatttttagtagagacggggtttctctatgttggtcaggttggtctcgaactcctgacctcaggtgatccacccgcctcagcctcccaaagtgctgggattataggtgtgagccactgcgcccggccgaaaatttatatttttaaagcgaaagctaacttctaattttgaaaatttttattgggagcaggatattataataataatctttagttgttaaaccattaaacatcaaggttttttacattgtttctatgcctcctccctcaaaaaaaaaaaacctcctacaataaaactgaaaaattgcacaaagacatattagtggaagaccactgcttcgtttacacaaatgaagagtataaagcagagaagtgctccttggggcaaaaggcaattggcaaaaagctaaggaacattttcataatgagttagaaatacagatcatcaggaatatccaggaagcgatagtgaataccaggcaggcttaagagacaggaaacatttagcatgttggtaaaccactttagcacatcagcaaaagcatataaacagctttaggattggaaattattgccatggggaaaggcaacaatttagaggagcaattccaattaaaaatataaagatcgtttctttagattaattcttgagttgctcccctatgcctgtgtagcagaatctaaaagataatcatgtgaacgggagattatatttaaaaaataaactttgaaattcataaaaaggaaaatgttaaagaatgtaaaattatactggtagaaaaaaaatttttttttttgaagatggagtttcactcttgttgcccaagctggcgtgcaatggcacgatctctgctcactgcaacctccacctcccaggttcaagcaattctcctgcctcagcctcccaagtagctgagattacaggcacgcgccaccacgcctggctaattttttgtatttttagtagaaacggggtttcaccatgttggccaggctggtctcgaactcctgacctcaagtgatctgcccacctcggcctcccaaagtgctgggattacaggcatgagccattgcacccggcagaaattttttaaaatagaaaaaaatagcccattcatgatacatgaattaaagaaaatattggtaacatgtatggtaacagattagtagctgagaacccaaaggctgccccaggttgcttcaaatcctggttctgccatgttccagctgtgtgaccttgggcaaactatcttaccctttgtttcagtttcctcatcaggaaaatgggctttaataggattattgtgtggattgagttaataagtaaagtgcttagaacaggcctccaaaatagtaagtgttgtatgcgttagctattattaatacatcacaaatttccactaaagaaaatcttccctagtttgtatatgggattgtcagaaaacaaatggaaaaatcatagtataaacagcctatatggctttaaagaattatataaaacacatgatctttctgaagtagttttttaaaagaaaaaagttagaactaacttctcccttgattttcactttttagagtggtgaaaccagaacaatatctcactttcattatactacctggccagattttggagtccctgaatcaccagcttcatttctcaatttcttgtttaaagtgagagaatctggctccttgaaccctgaccatgggcctgcggtgatccactgtagtgcaggcattgggcgctctggcaccttctctctggtagacacttgtcttgttttggtaagtgcttcttacgatctcattttaatctcattgatttttttttttaaattgcttccactggcagtgaataaactctgatgctgtatcccagaactgaggtttttcaacttaaacttctacttttggaaaaacctaaaaatctcatttccaccgttgcaactgcctcttattcatgactggtaggaatgtatccatgatgattttagagatccctaagaaggtgtttttcttcctggtagctttatattacagaaataataggtctctcccccacattttttcaaatataaattcatatgtaatattgaattccttcctcctcagacagatactttatctcattttttaaatttaaaaactagggataatgatagacatacttcacagggttcctgtgagaattcaatgaattaatacattaaggagacttagaagagtccctgatgttttctgagcattccataaatgtcagtattactgttactcccctgaggttcctgtctgtgcccccaacggctaattctttctccaccagtcattcccagtcccagcctgtatacagcctagatcttttcttctctttttaggacctttattttttcgttacttgggatacatgaaacttctgtttgcccttatacttctcctaacttctgtctttacttcttcctcttattataggatcaaacccaacaataatactgtctgctttgatttccatgccatctactccttcagtagtccctgcagcttagtttctgctttctgagcgtgttctaagattatcaataacctaagcagtttagcttgtcctggtattcagtctggtggggattttgtttttgtctttgtttagacagagtcttgccctgtcacccaggctggagtacaatggtgcaatcactgctcactgcagcctcaacctcctggactcaagtgatcctcccacctcagcctcccaagtagctgcctgcaccaccacacccaactagtttttgcttttttatttttagtagagacaaggcctcactatcttggccaggctagtctcaaattcctgggctcaagtgatcttcttgcctcagcctgccaaaatgctgggattacaggtttgagccaccacgcttgacacaagactggtgttcttaaccttgactttaaggccctgtattagtctcttcgatgtttctgtaaaacgtagctatgcttcattgcagttatgtcagcattcttatgctttccaccatgactgccagcacaaagtagattcccaggtgaattaatatgatgatgcttttcattgtaaacacacacacaaggagcttgatcaatggagtgatactcttggaaggagtgatactcttggaagaacatttcagacgttgcatctctaatgaaagctaggaattgggaacggttgagtcttaactaaacatctgtattactttaccactctgcgtatatatagtatacttcaatcaaaattagattgttacatcaaagttttaaaactctggtgattattttgttaatgacacctgttccctgccaactataacttcaacaattttttttcttttatttttggtgagcctgggtgtggggattctaacactaggtcagtagtccagagcagtgctgtagctcagcagtgttatcgtggatctgggcttttgctgtcttttccaccgtctttcttcattttattttattattctatttatttatttatttatttatttgagacgaagtttcgctctgttgccagggtggagtgcagtggtgcaatctcagctcactgcaacctccgcctcccgggttcaagagattctcctgcctcagcctcccgagtagctgggactacaagtgcgtaccaccatgcccagctaatttttgtatttttagtagagacggggttttaccatgttgtccaggaaggtctcaatctcttgacctcataatctgcccaccttggcctcccaaagtgctgggattacaggtgtgacccaccacatttttatttatttatttatttatttatttatttatttatttatttatttatttttgagatggagtctcgctctgtcgcccaggctggagtatagtggcactatctgacctcactgcaaccgccgcctcctgggttcaggcgattctccagcctcagcctccaatgtagctgggattacagacatgcgccgccacacccagctaatttttgtatttttagtagagatggcatttcgcgatgttggccaggctggtctctagctcctggcctcaagtgatttggcctcccaaagtgttgggattacaggcatgagccatcatgcctggcctcttttccaccattgttagatgggtgctgtatatccagacatcgtgtctgtagtgcagacatgaaggaggcggaaggggaatgagcagaaaggttgactcctgccatgacttttaaaaaaaaaaatagtccttatactagccagaactggttataaatccttcctttaaccaatgactggccagaaggaatgagattactatgattggtttggaatcagttgtgaatcagctaaagaatcaaagtcctccaactacttgaggggttgagatgtggggagggatgagtaggctgggatagttgttgtgtacataacagttatagctggagggttaaattatcgttgttctttcagaggagaaaatgcatagcaaaaatcttagcaatgatcttgcattgattctgtttcctgggttccaataacaagacttgtctcattttttgcctttcttgcagatggaaaaaggagatgatattaacataaaacaagtgttactgaacatgagaaaataccgaatgggtcttattcagaccccagatcaactgagattctcatacatggctataatagaaggagcaaaatgtataaagggagattctagtatacaggtaaacttcatacaggttttaaatcatatctatttgttaaatacacatagcatcaaataactgcactccacttgagcccttgtctttgtttcaaagagtgtatttgaatttttgtggtaaatccagctggctgtcttaaaattgtaaaattttagctagcttcctcatatatactcagcctcaggttaaatcctgtgtgttacttaaaagcccttgtagaaacaagatttcattaaagaaactgaatttttcattttgggggtggttaatcttttaattattaaagtaactggaaacttgagatacttttgccttcattaaacactgatacttggtttatgtgatatttgactttataacaacaagcaacttgaatatttatggcagatataatgaaattccagcaaaatgttatattcttctggttggtggctttattctcattaagctatttttactgcatatttgtagaacctaagttcaaagactgatcagaaacttgtgttatgtactaagttaaaatgtaaaatagaaacaaccacagactccattttttgtctggtttaatcacatagtattggacataagcagatactgtaagttgatgcataatataagaagatcgactgtcttttcattatgtttctcatgtgtgaacatatttactagcactttcatttctctgattactagccaatataagaatgtaaattagatcggaccattggtgtcagaataaaggtttatttaaaaagattttttccaaatctttaaatagcattaaaatattcttaatgcacagaaacattagggcactcagagccaaaatacgcaatgtttgggaaactcagcccattagaacatgtcctgcttcagacgccagactttgggaacactggatttgccagggtcactttggcagtgatcagtgatttaggtgaagtaaaagttatttcagatgtttaagattaggaaaatttccctttctaatcacagtgagctttcaccaacttaaatctggagttcaaaaagaaaagtagttcaatcagatttccccatgccttcacagcctgcatgccgccttccctgcctgcttcctggctgtgatgagacgggtaactgggagggaaatgtattgtcctttggattgtgtctttcctaccactgctttttttttttttaagttttgtgtctttagttttagaatacatactttatctgagggtggatctttttttatcacagagatgagtttttctggacacacttttttgtggagtctttgacagtgcagtttgtggctaggaaggattttttttctgttaatttatctggtgtgatattgtcctcaggctctcaattctaaggcattttctgcccctattccagataggatattggaaacatctttatccttcaggtactcaggatcagaaaagcatgctgcccattccaaggggttaatttctgcagaaatggtcttggcattctgagaatcttttttaattggtgcatttagaccatcggcatatgaagtgattactgataaagttggattaataaatacataccacacatgctactggtttttttttctttttttgagacgcagttttgctcttgttgcccagactggagtgcaatggcacaatctcggttcactgcagcctctgcctcccaggttcaagtggttctccccactcagccttccgagtagttgggattacaggtgcctgccaccactcctggctaattttgcatttttagtagagatggggtttcacagtgttgaccaggctggtctcgaactcctgacctcaagtgatcgcccacctcggcgtcccaaagtgctgagattacaggcgtgagccaccgcacccagcccaaagtgattgtatcttaagtctgtaaatatgtgtaattataatcttaaagcatgtattcatttacagaaaacttccaatgactttgacattcctatagacatataggctatttgcaattgcctttcagcacaaggtcaggcataaatacttaagctggttgctggtattaccacatatttattagcatattcccagtattgatgatgacagttaaccccccagagtaaatatctagtttggcaattcagaactaatgttgtaagatctctagctggtaatttgatattactaataaagtattaaatgtaaatgataaacttttaacaaattacagaggatttgaagtttaattgcattgcttaaagctttcttcagtgagagaaatgggtagagctagggttctaggcattgtgtggggggcatggctcctagtggcagtgactcatgggttggtgcaacgtgagcacagaattagaaagaaagatgaaacagtaccaggcctcaaaatgagagctcctttgaggttatagtttagtgaaatgtaattccttggcttcaccagtcagcattaatgacctgaggcaggtgactcaggagtgctggctcccttgtgtgtcacagggaggacagagacgagtgagtttgctgaacctatataggcatgagtgagtttgctgaacataggcacgagtgagtctgctgatcatagccatgaatgagtctgtcgaacatagccatgagtgagtttgctgaacatagccatggcaaaatgacattgttgggtttgtggggtttttttaattcactagcattaaatatctcttttatttctctctctgccgagtgctttccttgttgacgtattctgaatttgttgcatattctgggaagtggtccagtgagacttgatttttggtgtcatcttatttgttgacattgttgagtgttgtccagctataaggccaagttacataactaggtctcagtcaagagtggaaacttcaaaaattgaccagtgtaaattctggagacaatttaacatttttgtggggtactttgttgaacacacatctttataaggggtcacagcatagtcaacccagacttgcctaggttaaaattctgtctctactatttgttaggtgcatatccttgggcgagttacctaattattctccccacatcttccgtaaaaagggtgtaataatggtaatattagtattagtgcttatctcctaaggttattgtgaggattaagagctagcacatttgaagtgttagaatatggcatggagagtagcaagtactccatgtcttcactataaaagatttggattgagtaacaaaacaaacaagggaacaaagaaaatagacagtcatctaagtgaagacatccttaattactcctgtgttccattatattgtagtttcttgattgttggcacagtattctttcggggacatggtttgcgccctcttagggctcaggattttcagttctaccccactactcatgaagtccagagacctctttcccggaggtctatttcctttccagcctcatctctcctactcaagctttgcgtgttttccatcctctagacaaattgtaccaaactgtactactaactgttctccaaacacaacaaactgtagaatttcacattttctaaatcttctgtaaatcaactattataaaacaaaaaagccagccgtggtagttctctcctatagtcccagctactcaggaatctgagacaggaggactgcttgagcccaggagtttgaggctacagtgagctgtgatcacaccattgcattctggcctgggcaacagagtaagaccccatctctaaaaaaattaaaaacataaaaagaggtacattgattatgcagctaagtaaaatagccatatctgggaacggtgaagtgtggaaaatgcttttttttttgaaacagagtttcactgttgttgcccaggctggattgcagtggcgtgatctcggctcactgcaacctccgcctcccagattcatgcaattctcctgcctcagcctcccaagtagctgggattacaggcttgcaccaccatgcctggctaattttgtatttttagtagagacagggtttctccatgacaccgagtctcatctttcacccaggctagagtacagtggcacaatctcagctcactacaacctcggcctcccgggttcaagcaattcttctacctcagcctcccaagtaactgggattacaggcatgtgccaccacacccagctaatttttgtatttttggtagagatggggtttcaccatgttgcccaggctggtctcaaactcctgacctcaagtgatctgcccacctcagcctcccaaagtgctgagattacaggcatgagccactgtgccaggcgtgcctttttctttttaatacctctggtacatgatgacaatgagacaaattagaaactagtttatccattgtatgaatggatttcccagagattttttaaagatctgaatacaagcattgcttttaacagtaccatattgcacaagagtaactccagggaatgaatattccataagttctaaaaaactttaagttgcaatgttacatctctgtccaaaatcattagactcctcgggcaataagtagagtattccttatttattagatccaagactccttgcgtccttgataggatcttcaaagaaggccatccctgtccataccaagtcatttgggccagtgtcagtcataggtaatcaaaaccattggctcccactgcttctgcctctaccacttgccactggcagatacatagatggggatcatgtttcatcctttcaaggacttgagagactatagtcccagtgcataggttatatatgcgtttttcttctctggcatgtacaattgtcactgctttcatctgcaggggcccagatacactcttcttcctctacctttgtccctaggaatggtgacaagtgagagcacaggaaacatattttctagacatgatgctgggcgcttcacatgccatctcattaatcacaactgtctcccagggtagatgatgacatcctctttcatagaagaaactgaaatttgtcttcatcttggtccgaatcctggtttgtgttgccatctactcctgaaggccatgctagaggcatgctggagggtgtttctctagcccagtggtgggctagccagctgcctggagctgctagcgtacctgatagacacaaccaaattaaagtcctttagcatcacaaggaccagcttggagatctcaccgttagttctcccctagactgaacagtggcaaggagcaagaaggttccagatcacattcaagcaccactgtggtgactactccagtgttacatacctctggattagaagtggattaagattcaagcaaaatctaccttggctgaaatctttttaaaaatttttgtgttttttgtgcagtgctactttattttttattttgtttttttgtctgttggttggttggttcttttgagacggagtctcgctctgtcgcccaggagtgcagtggtgtgatctcagctcactgcaacctccgcctcctggggtcaagtgattctcctacctcagcctcccgagtagctgggactataggcgtgtgccaccacacccagctaattattttgtatttttagtagagatggggtttcaccatgttagctaggatggtctcgatctcctgacctcttgatccacctgcctcagcctcccaaagtgttgggattacaggcgtgagccaccgcatgccccggcctttgtttttaattgacacttagtaattgtacatatttatggggttcagtgtgatattttgatacatttatgtaatgaccaaattagactagcacgtctatcacctcatacatttatcttttctttgtggtgaaaacgttcaaaatcctctcttccagctattttgaaatctctatttaattatatattattatgtgctaatatatttcatagtacttgaaatatatataagtatatataagtatttcgaaatagccagaagagaggattttatgtgtatataaaatgtgtatgtatataaatttttttcgagatagggtctcattctcttgttcatgctggtgtacagtggcacgatcatggctcgctcactgtagcctccgcctcctgggctcaagtgatcctcctgccctagtctcttaagtaattgggactgcaggcgcgtgccaccacatatggctaatttttaaatttttgtagagactgggtctcaccatgttgcccaggctgatctcaaactcctgggctcaagcgatctgcccgccctagcctcccaaactgctagaattgcaggcatgagccaccacaccctgtctgaaatattaaatattattaaccatagtcattctgtgcagtaaaacaccagaactcatccccctgttgaactgttgttcagtttctcactatgccccctcttccctactctgcgaagcctccagtaggtactattctactctctgcttctatgagattaacatttttagattccacatatgagtgagaacatacagtatttgtctttgtgtacttggcttaatgtcctccagtttcatccattttgccacaaaagacaggatttcttttttttttaatggccaaatagtattatattatatatatatatatatatttttttttttttttttttccacattttctttctccctccattcattgatggacatttaggttgattccgtatcttgactactgtgaatagtgctgcaataagcacaggagtgccgatatgcctttgaaatcctaatttcatttcctttggataaatacccagtagtgggattgctggatcatatgatagttctatttttaatcttttaaggaacctttatgctgttttccaagatggctgtactaatttacattccaccagccgtgtacaagagttcccctttctccacattcatgccagcatttattttttgtctttttgataatagtcattctaattggggtgtggtgatatctcagtgtggtattgatttgcattattctgatgattagtgatattgaccattttctcatgtatctgttggctctttgtatgtcttttaagaaatggctattcaggtcttttacctatttttaattggattacttgtgttttctgctattgaattttgtttgagtttcttatatattctggatattaatccctgtcaaatgtatagtttgcagatattttcttccattctgtagattgtctcttcagtctatcgattgcttcctttgctatacagaagctttttagtttgatgtaatcccatttgtctgtttttgcttttgaggtcttatacaaaagacccttcccttgcccagaccaatgtcctgaagtgtttccctatcttttcttctagtatgtatatttgtttggggtctcatatttaagtctttagtccattttgagttgattttttaatatggtgagagataggggtctagtttcattcttgggcatctagatatccagtgttcccaacaccatttatcaaagagactgtccttcaccagtgtatattcttggcatctttgtcaaaaataaattggctgtaaatacgtagatttatttccatgctctgtcttctgttttattggtctttatacctgttcttatgatagtagcatgctattttggttactatagctttgtagtatattttgaggtcaggtagtgtgatgcccccagctttttgctcaaaattgctttggcttttggggtcttttatgatttcatacaaattttaggattatttattctgtttctgtgaagaatatcattggtattttcattgaattgcattgaatctgtagatcactttatgtagtatggacattttaacagtattaattttaatccatgaacataggctatctttccatttgtgtcctcttcaactttttccattaatgttttataattttcattatagagatctttcacctccttggttaaatttattcctaggtaatttttttgaaaatggaattgctttcttgatttcttttttcagatagtttgttagtgatgtataaaattctactgatttttatttgttgattttgtatcctgcaactttactgattttttttattagttataacagttttttggtagagtctatagggttttctatacataagattatgtcttctgcaaacagagacaatttgacttcctcctttccaatttgaataccctttctcttagctaattgctctggctaggacttacagtattatgctgaataaaagtgttgaaagtgggcatcctagcccggtgcggtggctcatgcctgtaatcccagcactttggggggctgagatgggcagatcatgaggtcaagagattgagaccaccctggccaacatggtgaaactctgcctctactaaaaatacaaaaattagctgggtatgctggcacgcgcctgtagtcccagctactcgggaggctgaggcaggagaatcgcttgaacctgggaggcagaggttgcagatcatgccactgcactccagcctggtgacagagcaagactccatctcaaaaaaaaaaaaaaaaaagaaaggaggcatctttgtcttgttccagatcttaagaggaaaagctttcaactttttcatgttcagtatgatattaactatgggtttgtcataacgtggctttgtgttgaggcacaatttcctccatacttaatttgttgagagtttttatcatgaaggtatgttgaatttttttttttttttgagacagagtcttgctctgttgcccaggctggagtgcagtggtgcaatctcggctcactgctacctccgccttctgggttcaagcgattctcatgcctcagccacctgagtggctgggattacaggcatgcaccaccacacccagctattttttgtatttatagtagagatggggtttcaccatgttggccagaccagtcccaaacgcctgacctcaagtgattcacccacctcagcctcccaaagtgctgggattacaggcatgaaccaccacgcccagccgctatgttgaattttatcaaatggtttttctgcatctgttgaaatgatcatatggttcttgtccttgattctgttgatttttgagagatcatgtttattgatttacatatgttgaaccatcctttcatcccttggatgaatcccacttggtcatggtaaatgatgtttttaatatactgttgaattcaatttgctattatttcattcaggatttttgcatccatattcatcaaggatattggcctgtagttttcttttttgttgtgtccttgtctggttttggtatcagggtaatgctggccttacagaatgattttggaagatttccctcttcagttttttgaatagtgtgaaaggaattagtattatttaaatgtttgttagaattcagccatgaagccatcaagtccgggcttttctttgatgggagactttttattactgatttaatctcactctttattggtcagttcaggttttctatgtcttcatggttccgttttgatagttcgtgtgtgtctaagaatttagccatttcctctaggcttttcaatttgttggcatacagttgtttataatagtctcttacaatactttgtttatggtattagttgtaatatctcctttttcatctctgatttgagtcttttttttttcttagtctgctataggtttgttgattttgtttgccttttcaaaaaaatgatccttggttttgttgatctttcgtttcttttttttggtcactttatttccattcttctggtctttattattttctttgttctgctaattttgggtttagtttattattttctagttccttgaggtacatcaggttgtttatttgagaactttctttttcttttttttttttttttttttgagacagtttcactctgacccctagactggagtgcagtggcatgattttggctcagtgcaacctccgcctcccgggttcaagtgattcttatgcctcagcttcccaaatacttggaattacaggtgcccaccaccactcctggctaatttatggtagagatggcatttcaccatgttggccaggctggtctcaaactcctcacctcaggtgatcctccagcctctacctcccaaagttcttgggtgtgagccacccacctggcctgattgtgatttttttctttttttaatttgttgagacttgtttttgtggcctaacatatggtccatcatggagaatgttccatgtgctgatgagaaaaatgtgtattctgtggctgttggagggaatgttctgtaaatgtgtgttaggtccatttggcctagagtgcagtttaaatccaatgtttttttgttgattttatgtctggatgatctgtccattgctgaaagtgggatgtagcgttctcctgctattattgtattgcagtctatgtctccctttagatgtaataatacttgctttatatatttgtgtgcaccagtgttgtgtgcatagatatttaaaatcattacatcctcctgctgaactgacccctttgacctttgtttctttttacaatttttgacctaaagtctattttatcaaaatatagctactcctcttctctttttggttttaatttgcatggagtatcatttctcatcccttcactttcagtcttagtgtgtccttacaggtgaagtgagtgtcttgtaggcaacatagagttggattttgttttttgttttttcatccattcagccactgtatcttttcattgaagtatttaatctatttacattcaagggtattattgatagataaggacttacacctgccattttgttaattgttttctgtttgcttttcatattctttattcctttctttctctcttattgtttacctttgtggtttggtgattttctatagtgataagctttgatttatttctctttctcatttgtgtgtctgctgtagttactttttttctttgtggttactctggggcttacattaaacatcttagagtgttttaagctgataataacttaaccttggtctcataaaatatttttggcttttaccctccccccacaacttataattttgttgcttaatttacatctttttatattgtgtggtccttaacaacttgtagctgtagttaccattaaccattttttacttttaaccttcataatggagatttgaaagattatattccacagttacagcaatggagttttctgaatttggtaattaatttacctctgtcagtgagattatattttcatatattttcatgatagtaattatacttttgcttccagtcaaagcagctcccttaagcatttcttgtaaggctagtctagtggtaataaattcttgcagcttttgcttgtctgggaaagactccatttttctttcatttctgaaagatagctttgctagtagttgttatctttcagcactttgaatatattatcccattctctacagtctgttctttcttggcgtgtacaggttttctgagaaatctgttaatagcaacctgccaggttcccttacatgtgacttgatccttttctcttgctgcttttagaattctctatgtctgacttttgatggctttattataatgtaccttggagagaggacctctttgcattgaatgtgttcagtgtgctgtcgagtttcatggatctggctaccaatattaaaattatattactgtgactaatagttgagtgtggtcccagtatagtttgtctcaaataggtaaaagtccacaagattttattaagattattatattcaactatatactcatgtacataaatactaaatgctttattataaaattttgaaattaaaataagcatagttgttacaagctacagtatggcaaaatgcctttttctttttcactcatcttttagcatcattgggttttcttaacttgaatataggattttaattcataaatttttttatttaaggaaaataaatttttaatgtttgcatttgtttttcatttctcttccctacctagaaacgatggaaagaactttctaaggaagacttatctcctgcctttgatcattcaccaaacaaaataatgactgaaaaatacaatgggaacagaataggtctagaagaagaaaaactgacaggtgaccgatgtacaggactttcctctaaaatgcaagatacaatggaggagaacagtgagaggtaagctttctataactacaggctgaaagatgaggcttttttattttaaaaataaattttatgggaccaggtgccataggatttcaggtatgtaatttttatattaggaaaatacaatcacgcctgtaatctcaacactttgggaggccaaggagggaagatcacttgagtctaggagtttgaaaccagcctgggcaacatagtgagaccttatctctacagaaaataaacaaaattagccaggtgtggtgatgcgagcctgtggtcccagccactcgggagactgaagtgggaggatcacttgagctggggaggtcgaggttgcagtgagctgacataccaccactgcactcccacctgggtgacaaagcaagaccctgtctcataaataataataataaataaataattatgtggatatgatatatgaaataatataggcccttttttttattgaattaatataacctttttttttctctgtcacccaggctagagtgcggtggcacaaccttagctgctccacctcctgggttcaagcaattctcctgcctcagccctcccatgtagctgggattacaggcgcccaccaccatgtccagctaatttttgtatttttagtagagagagggtttcaccatgttaacaaggctggtcttgaactcctgacctcaggtcatccacccgcctcagcctcccaaagtgctgggattacaggcgtgagccaccgtgcccagccaaattaatataacctttatttcttacctgagggtatttagtatatgatgacaaaggtgttcttaaggagctttcagccaatacttagaaaccagttcaaaaatcctttaaaacaataatgattgagtgtaaaagttaatataattgggtgctaaaatatatggtatggtacagaaaataagtagtatgaatacctgccatttgtactataattataatgtaactgataatatgcctttaataaaacatttaaacatgtaagcttaattgcattactggagaaagaaaaaggcgttttttaaaaatccatatgaatgtggcatctattcagaattttaaattacaattacatttctaagactgtctctcacagattaggaagctgataacctggtataagtcacatagcaaagtgaatgaggtttttctttcctctctcctgcatctctgaataacttttcttcttctctttttgccaaatatacttcttatcatggggaattttatcttgtggttatatttgttagattggaatgaaaatagtcgaagtctgccatacattatagctttactgctgtattttcctaatataaaaattaaatacctgaaattttataaatgctaacatattccaatatataatacatgtattattatatattatagtggaagagtttattatgatttagtaatgttattcccaatttttgacttcagtagttttagactgcaagagaaagcataatttttttggttccttttttcatcttaaatagaatatctttaaatcaggaaataatagttttaagttaccttagctttttcaataataaggctttcagattcctagtgttactgctttttccccccttttatctagtactatctgcagaattaaatgaaaaacaggcttgatgactgaaatggcattcttagtatttcagaagtgttgagttttaatctacaaagattcagaacaaccatgttaatcctagtgagttaaatcactcatacaagcttgcaaatacagaggagactactaaaaccactctagagtctataactctaaggagttatagagtgattgcagaagactgttttatggggtctgtgtgtgtgtttatgtgcctgtacatgttttcgcatgtggtggcggtgttttcttctttcccttttttccttccctgtctgataaatgagactggtgcatgtttgaaagcagagagtcccaacagaaaagtcagaattctaagcggagaagtctgattacaaaaacaaggttacagaaatgaaaaagaggaggccgggcgtggtggctcacgcttgtaatctcagtgctgtgggaggtcaaggcaggtggatcacttgaggtcaggagtttgagaccagcctggccaacatggtgaaaccccgtctctgctgaaaatacaaaaattaaccaggcactgagagtgagaaacaacttggacatacgaattttaaaatacaaaaattaaagaggcattaacgcctgttgtcccagcttcttgggaggctgaggtgggagaattgcttgaacccaggaggttgcagtgagccgagatcataacactgcactccagactgggcaacagagcaagactcttatctcaaaaaaaaaaaaatgtaaaagaagagcataggatcagggtgtgaagtaaaggttactctgaacagttttattttttaatcagtcccagtaatttttaatacctatagatagctttgcattaaagatcctcataaaagtcttatcaaaagaatttcaagtttcttttttttctttttttttttcctgagacggagtcttgctctgtcacccaggctgaagtgcagtggcgcagtcttggctcactgcaacctccacctcccgggttcaagcaattctcctgccttagcctcccaagtagctgggattacaggcccccgccactacacccagctaatttttgtatttttagtagaggcagggtttcaccatgttggccaggctggtctcgaactcctgaccttgtgatcctcccgcctcagcctcccaaagtgctgggattacagacatgagccaccacacctggccagaatttcaactttcaaaagaaatagaacacagtactttcttgtattcattttcattgtattcatttggaggatattcatcctccaaagcagtgtttttcaaattgaaggtcaaatcacatagtcgtgaagtttattggattataaacattttttaaagaagaaaatagaataaaatagaaattatcagcatatcacaaataataacatgaatttaacagatgctggcgaggttgtggagaaaaaggaatgcttaaacactgttggtgggagtataaattagttcaactattgtggaagacagtgtggtgattcctcaaagacctaaaaacagaaataccattcaacccagcaatcccattactgggtatacacccaaaagaatataaatcattgtactgtaaagatgtaaacacacgcgtatgttgattgcagcactattcaccatagaaaagacatggaatcaatctaaatgcctatcaatgatagactgcataaagaaaatgtacatatacagcatggaatactatgcatccataataaagaatgagatcatgtcctttgtaggggcttggatggagctggaggccattatccttagcaaaactaatggaggaacagaaaaccaaataccccatgttcttactaacaattgagagctaaatgatgagaacacatggacacatagaggggaacaacactgggggcctgtcggaggatggagggtgggaggagggggacaatcaggaaaaataactaatgggtactaggcttaatacctgggtgatgaaataatctgtacaacaaaacttgttgtacatgtcacggagatgacacaagtttcaagcttatgtaacaagcctgcacatggacgtacccctgaacttgaattttaacctgtttttatatatgtatgtgtgatgtataaattccatatatttacatatacattgtgtacctatgtaagtgtgtgtgtgaggttactctgttgtgatgctatttcttactgtaggttgcagtctgaatttgaaaggcagtattctcaattacagttcaacccagaaacccagatcactgagagtgagaaacaacttggacatacgaaaagtctggagcaggcagagtggctcatgcctgtaatcccaacactcggggaggctgaggcgggtggatcacttgagcctagcagttcaagaccagcctgcgcaacatggcaaaccctgtctctacaaaaaatacaaaaatccgccaaatgtggtgggacctgcctgtagtcttaagggaggctgaggtgggaggattgcttgagcccaagaggttgaggctacagggagctatgatcacgccacatgatcttccctgggcaagggtccacaggaggcattcccattggttaatatcctaaattgcataaatatacgggaaaacctgggcaggtcccatttggcaagtcattacacagacataatttctttaggccccactaaggaagaaaagattttatcacaaatcgagagtagtcttgtcattctctggaattgtgttcttaaattagatacaggcatgccttggtatttgtctctagtccattcctgaattatatagtgcacttttggaaagcaaaaacctctgttccattcttttaaatgagaaaaatagtgtgttgtcagcccaaccagaaagcataaccagttcccccaaatatagtacaaaaccttttaaactcctatgtagcatttaaagcactaattatgtaatcatataacacttaagggactcattattgggcttgtatggtaaatgacaaaatactagatacaaattataattgccttctataccgcaatgaaaataaaaaatgaaataatacgttaaagattatatctaaattggaaccttgttaacgatacccccaggctcctggcaaaagctaataaagggcccatgggaggctaaccccctctctgtttttatacatattttgcctacacttgaacacaaatgttttcactaacaggtacatgcatatttgtgatagtgaattttcaattcagagagtgaattctgagtaagaatttttgcaagtcattatagcagatattcagatggagttcagacagccagggatacctgtatatatgataacaaaaagtgaaaaagacatgagaaagcaccataagatgaacaaagatagcaaaacccagtctgtcctcaagttgcagaatatgtgatgaagataaaacaagtacccaagttagcacattattagtaaattataaattacttcaaattaaatgccgtaagaatagaagggctagagggggtggagcaagataattgaagagtagaactgaaatgagaggacaggagagaagggaaggtttctattttagtggggaacaaatttggagaacagcaaaaaaacggcaagaacaaagttctgcatagcaggagaatattggcaagcgtttgcatggacatgtgttctcagttctcttgggtacatatctaggagtggaattgctaggtcatatggtaactctatgtttaactttttgaggagctgtcaaactgttttctgtggcagctgaaccattttacattcccactaacaatctcttggggagagaaggttccagtacaccaaagaagtgtaacattgattaaaatattctaatacatagttcaattcagatttctcttgtctgaaagattcaccatatagcttttttaataaagttttatggaaatataatttatatagcataaagtttactcatttaaagtatacaattcagtgacttttaatatatttaccaaattgtgtagctggcaccacgatctaattttagaacattttcctgatcccaacttctgtagctgtttaaaaaatatttcaagattaatcaaagattgatactatatttagttgatgtgattctttggtctcctttaatctggaactgttccctgcctttttcttccttgcttgacatttgtatttttgaagcttctaggccagatatcttttagaatgccccatacaatgcatttctttaatcattatttatcatagttaaattcaggttaaacgtcttggccaggaaaaaggtagtctcaatgtaggagacattgaagaggcatttgagatttttaagcaccatcgtgcactaggcccggtaacgaaaaaaaaaaaaaaagattttaaatcaagctacgtgtccaggggtatgctctagaaaaattaccatggcaataatatttaatttgagggcagggaagctggttaggcctttgctgtaatacagaaaagtgaaaagagggaatggaagaaatagtaacctatatgttgccattggccgtttttttgtttttgtgggtgtgcttttatagagaggcaagacttgctatattgcacaagctggacttgaattcctgggcttaagccgtcctcctgagtagctggatggcattattatttatatagaaccagaactaagtatcttgagagtcagagaccttccactgaaacaaaatttctgagaccggacacagtggctcatgcctgtaatcccagcactttgggaggctgaggtgggcggatcacttgagatcaggagttcaagaccagcctggctaacatggtgaaacatcatctcactaaaaatacaaaaaaattagccaggcgtggtggtgcacgtctgtaatcccagctacttgggaggctgaggcagaagaattgcttgaacccggaaggcgggggttgcagtgagccaagatcgtgccactgcactcccgcctgggcaacagaacaagactccatccaccccgcctcccccaccaccccaaaaaaatttctgtattgactatgaaaatgggaggtagaaatgtgtatggtcaaataaaagaataagtgctcccacaacagaatattcatggaaatattaaatagctagcttttataagagccttaaccatcaaacgtgaccaggaaatgggatcagatgcaagtggtacccccttgtcttcccactcctgctcctgctgactagttaacaaggaaggaaagaaaactaacagtcatcaattcacgtgtcaagaaagtaccttgcttattttcaggtcccactaaaagcagttatcaatataaaactgtaccaggaatggttgtcaaggtacatctctgaatcattaaagaatattgaggcttttcttggccgggcgccgtggctcacacctgtaatctcaggactttgggaggccaaggcgggtgaattgcttgaggtcaggagttcaagaccagcctggccaacatggcaaaaccctgtctctactaaaaatacaaaaaattagctgggcatagtggtgcacgcctgcagtctcagttacttgggaggctgaggcacgagaatcacttgagcgtgggaggcagaggttgcagtgaaccaagattgtaccactgcactccaggctgggtgacagagcaagacgctgtctcaaaagaatattgaggcttttcccctcattattccagtgttcaaatagggtgaaaactatgtggatggttttatttgcgcctaggtcctctgtgttagagtactcaagtcctgctctgtgtgcacttcctttcacttgtttttatttcctcatagtgctctacggaaacgtattcgagaggacagaaaggccaccacagctcagaaggtgcagcagatgaaacagaggctaaatgagaatgaacgaaaaagaaaaaggtggttatattggcaacctattctcactaagatggggtttatgtcagtcattttggttggcgcttttgttggctggacactgttttttcagcaaaatgccctataaacaattaattttgcccagcaagcttctgcactagtaactgacagtgctacattaatcataggggtttgtctgcagcaaacgcctcatatcccaaaaacggtgcagtagaatagacatcaaccagataagtgatatttacagtcacaagcccaacatctcaggactcttgactgcaggttcctctgaaccccaaactgtaaatggctgtctaaaataaagacattcatgtttgttaaaaactggtaaattttgcaactgtattcatacatgtcaaacacagtatttcacctgaccaacattgagatatcctttatcacaggatttgtttttggaggctatctggattttaacctgcacttgatataagcaataaatattgtggttttatctacgttattggaaagaaaatgacatttaaataatgtgtgtaatgtataatgtactattgacatgggcatcaacacttttattcttaagcatttcagggtaaatatattttataagtatctatttaatcttttgtagttaactgtactttttaagagctcaatttgaaaaatctgttactaaaaaaataaattgtatgtcgattgaattgtactggatacattttccatttttctaaagagaagtttgatatgagcagttagaagttggaataagcaatttctactatatattgcatttcttttatgttttacagttttccccattttaaaaagaaaagcaaacaaagaaacaaaagtttttcctaaaaatatctttgaaggaaaattctccttactgggatagtcaggtaaacagttggtcaagactttgtaaagaaattggtttctgtaaatcccattattgatatgtttatttttcatgaaaatttcaatgtagttggggtagattatgatttaggaagcaaaagtaagaagcagcattttatgattcataatttcagtttactagactgaagttttgaagtaaacacttttcagtttctttctacttcaataaatagtatgattatatgcaaaccttacattgtcattttaacttaatgaatattttttaaagcaaactgtttaatgaatttaactgctcatttgaatgctagctttcctcagatttcaacattccattcagtgtttaatttgtcttacttaaacttgaaattgttgttacaaatttaattgctaggaggcatggatagcatacattattatggatagcataccttatttcagtggttttcaaactatgctcattggatgtccaggtgggtcaagaggttactttcaaccacagcatctctgccttgtctctttatatgccacataagatttctgcataaggcttaagtattttaaagggggcagttatcatttaaaaacagtttggtcgggcgcggtggctcatgcctgtaatcccagcactttgggaggctgaagtgggcagatcacctgaggtcaggagttcaagaccagcctggccaacgtggtgaaacaccatctctactaaaaatgcaaaaattagctgggcatggtggagggcacctgtaatctcagctactcaggaggctgaggtaggagaattgcttgaacccaggagatggaggttgcagtgagctgagatcacgtcactgcactccagccagggcgacagagcgagactccatctcaaaagaaacaaacaaaaaaaacagtttgggccgggtgtggtggctcacgcttgtaatcccagcacttcggaaggccaaggcgggcggatcacgaggtcaagagatggagactgtcctggccaacatggtgaaatcccttctttactaaaaatacaaaaattatctgggcgtggtggtgcatgcctgtagtcccagctccttgggaggctaaggcaggagaatcacttgaacccgggaggcagaggttgcagtgagccgagattgcaccactgcactccagcctggcaacagagcaagacttcgtctcaaaaaaaaaaaaaaaaaaagtttgaaaaccattggtatagatagatattttgaattgatttgcatagtctccttgaatgtgttaaattatgttgaaagtatgaaagcaggatgtaggtggtactacatattaaataagatttatataacatgtgcctgtgtcctgattcagtattactgtgtcctgtaaccttgaaatggagaggaggtaagagggacatttttttatgtctgtctgcattaaacctgtgttgaggcatccaagacccttacatttcaggttgttattggtttaaaacctctttccagtttgcattggtttgtctttgctaaatgcagtatgtttatgttactctgcaagtcacatacaggtagaccttatgacatttccagttgaaactaggcttgctttcggtacacaggtgaagacttgaggaagccagtgccagagaggacgtgcctcacccctcagctggctgaatgcgcttcattggcttacgctttgctttctgtatgctttctgtccttctgggtggtgtcgcatatatttttaaaaccacattcagtgtgatgaacacttgttggtacacttctaaggttagtcatctttccttctctgagcagatccttgagaagggcattgacagtggatgctttaaatagtgttcagtttttaccagtaataatattttttggtggatgtcatgaaaacacatgacatcgtgttgtgcccatgataaaaagtcctgactttttattgaatgaggtgtgctggtcaaagagacagacgtggctgccttctctggtaacagccagaggaaaaagttagcaattctagatgggttctacagctttaaaaaacttgttgtatccttaggtcctcataatattaaatattttctaattttaacagattttaagagtcagatatatactttgacttgaatgatgtagtgttgcttaaggaagaaaaaattataaaaaaaatgtttcttttattttcaggagtttttaattcatatctagttcatttccactcatatctatttctgctaatagattgacagcaacctttggtttaatgctgtgttctctagctacaccaacaagttatatattcctttccaatattaaccaagtatttattttatgttagcctatgactgagttttatctatgcttgcacatatatagaatgtaaaaatataattctccatgtttaatgtatccatataaaaatgaaaatacccattttctgcattcttaaagggttaagaaaagtaacatgtaataagcacatggaggactttagaccatttcttgtgttagaggagactttatacatttgtgaggttttctgtcttttggtccttttatgttcttagtccatttcattattgttttaatatttctaccaaggaatgaccaagaaaagagaaaggaagaaaaaaaatgtgttaatcattttcttttaatggtatgtcagcagaaggggttctgactgtaagctaatgtgcacttgtgcctgtcctctttgtcttaaactaaagttggtggtatgaatcgaaaaatgtgtattggtttaaaaagcagtttatgatatgaaacagtcttccagtttttatatcaactcctttgctttcaacagtcagttcaaagcaccatttattgatggtttactgtatgccagacactgtattttttctttacgtattaccctacttaatcctcaacacaattttatgaagtagttattgttagcctgaaactggcatttctccatccttttatggtgctgtctttgaaaagtctcagatgtgtaacatgtcctagaagtagataattatcaaaggaagactgcaactgcaactcacttccctctgagaaaaaatatccctgactagattacatatctagttccagaccagaacctggtggtattaaaagtgttttggttgactattgtataccataatgagtacctacattcccaaattgcctgcctggttgttgtacagacttaaaaatctagcctgccttgatttttacttttcagatacacatgtttaaagagatctttgctggtcacagtagctcacacctgtattcccagaactttgggaggccaaggtgggaggattgcttgagcccaggagtttgttcaagatcagcctgggtctctacttaaaaaaaaaagaaaaatttagctgggtatgttggcacgtgcctgtagtcccagctactcaagaggctgaggcaggaggatcacataagcacagaagtttgaggctgcagtgaactatgatggagccattgcactccagcctgggcagtagaggagacttgtctctctctgtatatttgtctctctctccctctctctctctgtctctcataatacatatatacatacatatatatatacacacacacacacacacatacagagagatatataaaatagatataaaaataaagatattttaaagattatttataaatatatgttttttaaaaatgtagtctattataagccaaatgtcattaggcatgtagtccatttctttctgtgttcaggcaggagttccagttctataaacattggtttatctgtttttctagcaggtgcacacatgggttctcagtgatgtgtagccggggcatagattgctctcagtctgttgcaccctgcaggggagatgtctgaggtcctccctcgcctggctgtgctggacgcacagtgcatagcagaactaactgcacagcagaaatgcctgttctgcgttcccattgatacaccaacagctaatgaagtctcttttacttttaagaaacccttatttggctttggcatatttacatggtagaggcctagaattttataaataaaattttgaagaatagaaatgtattttttcacgatttaaaaaccacctgtatggtttactctggagttgacctgttccactcatccttctccccagttcatttgtttcgtttcaattccactttcccttatagctatcttcaaagcagtgtctatgggacagcattgccaagagccctgactgggcctggcttggtaactcactcctgtaatcccacctgcccctagaagtgcagatatactagaaaatacagctgtctttgttccagtgaatatgtataaggggtgacatgccttggtggaaagcagctcagattgtgtggactcctcaatccctgctctaccaagagctctccagttccaaaactgtacactcagtggctatgttctgtgtgcctgctcccaatacagccattatgttgttcatccagatttgagaatttaagcttttcactcctctttgggtagtattatttaagtaagaacttctaggaaaatagtatgagtttggttatttttccctcggcagcaggaattttctgaatatttagcacattcagattgctccaagaacagcattgagcaatgctgtacatgtgctttctcctaaggctgtctcccctgtctactgggttctggaaagactcattgtccttcagggaagggagcatttttcctcactgccattgctgagccttttctcctgttgtatactatttagcccagcatgaagccatcgccgactgtaaggagtgatggatagaagtgtttacttggtttgtgaggccttgtctctaaagggtaacagtctgacaaggaaaacatttcaaagagccctgaccaggcctggcttggtaactcactcctgtaatcccagtactttgggaggccgaggctggagaatcatttgagcccaggggttcgagaccagcctgggcaacacggtgaaaccccactctatttaaaaaaaaaaaaaaaaaaaagccctgatccccctctctgggcgatccattgggagaatttgaaaagttcaaacttttgaactcttcctactttatttttttaaatttagaatggaaggtgggaattatgcaatttggtgaagatgttacagaaactgcttcacgactttctggttttacgttatacagtatgttttgaggagctccaagaattgcacaggaatttttttctttatacagaccattttttaaagacgcatcaagttattctttattgtggctctaatttggcatatttttcagacacaggttgctcttgatacatcaaatttctgctgttaaaaacttcagttattttgagtttagttaattcaggagcgggaacctaggtaagtcatctgtagagcatctttgcaccttaagagaagtttctgattagcctcaattcagtttagtagaatattagtgtggatatctggtggcaaaacctattcttggtgcaatttggcttttcgtggcctcaggcatagagctgacttggtagtggaccctctgtccacacccacccggcccttgcgcctttctagcataaagtggtgtgtcagagccactgtctccacagaaagcaccacgtttgtttcatttgacttatttgaacccgtttctcctgcctttgcctttttaaataaaaatagcaaaaattgattcaagtgaatctattagaattttctaaaatggagcccatttgtcttttcagtcttgcaagtaaagtctttaaaacaattaagcctcccaatgatttaaccgtattttcttacccccccaccctgaataatatttacaaagaaataataacgtgaatttttaaaaaccccgtccttcagttgttataaatgatagttcattcttggtaagtatatctgtaacaaaatactggggaaggacatgaggtatggtgaacacttaaaattctgccagccagcttaaatacataatctaaatttaacccacgtaacacctcttctaggtaaacagcattctccctatattcagatgagaaaactgagtcaaagaataagtaacttgcccaagattgcacagctagtacatgacagaactgagattcactctgcagtctgtagacttccagtaactccaaacctcatactttttcttttatgttacacggactctgtttttaatggaatatttaaatgagatatttaagtgggaaagtaatagacaaccactcttacctgttaaaatccaaagtaattctctagtttttaaatggagcacatttaaaaaactttgtgcaccagtttgaactaccactgcaacagagaaatactagaacatcagagtattctctaaaccagatcattacttcattcactttgattgaattgagttgcttattttaaaaagacttctggatgcatggaaaacatatatattttgcagcactcagaatcaatacaaagtaccttcagccactttggttcacattctgaagattcatgtaataaacacccatagtagttgtgggctcacattatctaatgagaatgttttcttgatgacttgacatttctccgtcactgttggataatcagagaaaaaaaggacgttcagaacaccaaggtgcttggtacaattatgccaagtgccctgtctttaaagggagtagcctaattctcagcatttgggagtttttgactaaatgtgtggatatgatttgtatttcctatttcttaatatatatgtgaagatacaatactgacaagtacttagatttggaggttttgcggtactgaggggagttgggggaagaatggaaataccattgacctcctaaaaagttgttacttgcaaagtttgggaggtgacatcaaaaactcaactgcccttacaatagtcattccatccatctgttgcttatttgaattctcatttatttttactttatggcattaaaatacaataaatctgtcaattatgtattttatattagtagtagcttaagattgggtcacttcatttcggtagatataattgttagtattatccttcaggacaaaaagcatctgctaacaacctgtggtttaaaaatataggccaactttatgttcaaacattatgttgataatatttttagcagtattacacagtggaggtccaaattggattagacttttgcattgaattccaaagtattggtaagtctagcacatagtataaagtcttgctaaattcttgtgggtacattatttttaaaacctgtctgtctgtcggtatcacacagaggtcacttcttgagtaaaataagatgaccttaagaattcacataattcttgaagaagaatataagtcacgatgcttccacatgaggccgcgtcacaccaccatcgtttggaaatccagcaccccagccgttttaccctaagataacagtgcagcacttagtgtggttgaatgtgtcatgtgggaatgatttttggtcaccttttcagttcattgttagtccttttatgtttgtgtgcatctatgttcgtctgtgtgtttggtaaatatgaattgaatagatgacttcttattttatgttttaggccaagattgacagacacctaatattcatgacttgagaatattctgcagctataaattttgaaccattgatgtgcaaagcaagacctgaagcccactccggaaactaaagtgaggctcgctaaccctctagattgcctcacagttgtttgtttacaaagtaaactttacatccaggggatgaagagcacccaccagcagaagactttgcagaacctttaattggatgtgttaagtgtttttaatgagtgtatgaaatgtagaaagatgtacaagaaataaattaggggagattactttgtattgtactgccattcctactgtatttttatactttttggcagcattaaatatttttgttaaatagtc

SEQ ID NO: 4 - DNA, 59258 bp, Mus musculus
acctgggcagtctctggaccacgcccccgcctgccccgcctcctccacgacgtccgacccacgcggcgcccaggcagcgcgctgacgtccgacgttccaggtactttccccaaggccggcttggctcaacgtgggggggcggggcggggcgcggagcgcgcatgcgccacagtgccagcgctctccccggatagagcggggcccgagcctgtccgctgtggtagttccgctcgcgctgccccgccgccatgtcggcaaccatcgagcgggagttcgaggaactggatgctcagtgtcgctggcagccgttatacttggtgagtcttaggcccgcgcggcgtcccggggcttccgagggtcggagctcgcgcctgccggctccgtgtcacctcccccgtctcccgctcgggcggaagtggcgagcgcgggccgcgtcaggggcgcgcctgcgcggacagtcctcaccgctgtgaggcttagacctgaagcaccgaggtgccggtcgcctgttggttctgaaggcggcgccacagcggcgcctgctggcggcccggccggcctcggcgtgtggtgcgccgggcggagggtcgaggggcacccggcgcggggtgggacgggctccggcttcctcgctgaccgtgttcttgtcggccgaatgttcggtggccgagcgcgctggtagcgggagcagcgagaagggcgagagagccctctaggatcggggccagcgaaggcaaagcggggtcccacggagtctcggtcatttgcgggtgtcccgggggcctgtggccggagctctataccagagccggagctgaagcggcggcctaagctcctgagggatcggggatcgccagtcttttcattggccactttctctgggagagcgggtcatcttctgcttagagctccccggaacaacaggggctttggttgtattaccattgattaaagtagcgtcctctttagaatttcctttgcttaggcatactttatagatgtatatatagtacaccacaggaatttatatgagccttgcagttggttgcatttcccaaatgagtgcactggtcgtcgtagcactcaaaagtaacccaacaatctttcggtccaaaagaggccacatgtctataggccgccgctgtgctctgtcaccagagaataaccttacctattcagctcattctaaagtattctttcctatgaacgcttgtgactacctttttacagcgtgtgggctaccttttaatccaggcaaaacttttctccccttaaaaatgattcattgctaatcattaacgtctatcccagatcagatttagtgattggtgtctccttatgttttggcttgttactacagtacagaaagaagctagaaagctttcttttagctctggatgactctgggttttcaccatttcatttaattagatggcagtaaaaattcgctttgtttaggacttatttctcccatcctcctttgagataaggtctcacagtctataacttttgctggctcagaacttgctttgtagacctgcctgaccttgaactcctaggctggctttgaagtcctagtcttctaactaccagtgtcaaagttctggaaagaagttgtagaaaatcttaaatgcaaattctagtggatttgtgtgtgtgaacttgcaggtctgtgggtacattagatttagcagagtgccgcctcaaaaatgccaatgacaaaaggggtactaaatcaaggaagattgctgggtggtacatgcctataaccccaaagtttaagaggcagagggaggtctgtgaagtgattttattttatttatgtagccttggctgaccttgaactcagattgtcctgcctccccagtgctgggactaaagccgtgcacctccatgtccagcacagctctgagtttaggctagcctggtctgcatagttccagaccagccagggctacatagtaagatcaagtatgcatgatcttatatacatacatacatacatacatacatacatacatacatagaggtgggagggggactagagagatggctttcatttaagagaacttgttcttccaggttgattcctagcacccacaagcagctcacaactgtttctagctcctgttattgggaatccgaagcactcctctggccttcagggatgccagttatgcacatggtgcctaggcagacaaaatgccatacactcagaatcaagggggaaaagtaaaataataataataattactattgttactactactattattttaaaaacaagggttcatactagcaacttaatttagcctaatttattttctgagaccaggtcttgcttagtatcacaaactggcctataatttaccactaggtttggatttgtgacagtccttttgccttagtcccccaattgctaggattatagggaagagccaccacatgtggcttaattgggtacatacatgtaagtcattaatgtctgagaagtaagtgaaagttgtgatcccacttaacagggaaaaaaagcacttatgctggatggtggctcagcctgtaacctgtcaattaggctgaagcaggaggattgttgtgagtttaagactagcctgcagctgggctttggtggccaagcctttaatcccagcagagtcaggttgatcttcagtgttcagccagcctaggctgcatggtgagttctatgctacccaggactaaggaatacattcttgtgttggaataaaaaatttctaaacaaaagccagtcatagtggcatggtttttttgggaggggggcatgtttcgagacagggtttttctgtgtagccctggctgtcctagaactcactctgtagaccaggctggccttgaactcagaaattaaaggtgtgtgccactacagcctggggtggcatgttttttaatcctagcaccgaggaagtggctccagaaagtatctggagttttaaaggtcatctccagctatccgtggagttatgggtcagcctgggataatcgagactcggtttaaaagtaaaaatcttgctggcaaggttgctctgtgggttaacagttgcccagcctgaaaacttgagttcagtcctgtgattcacaaggagggagagaaccacctttctttcacagatcttcgtatatacctgcctttttgtgtgcagccacaatagcgaagttaaaatgtaattgtaaacaattggatgttcagggagatggcttatttgtttagtgtttgctgtgaaagcatgaagacctgtatttagatcttccccatttccataaaaagccaggtgtagctctaatcttttgtgctggggaggtgaagacctgcaaatctctggagctcactggtcagcgggcctccctgaagcagtgagctccaggtttaatgagagacccagtttcaaaagataagggtttgggggctgcagagatggctcaggggttaggagcccttgctactcttgcagaggacactagttcagttcccagtgtccacatcaggtatctcagaactgcctgtaagttcagtgccagaggatctgacatcttctgtcctctatggggagtgcaatcctgtgcacaaacacatgtttacacataactaagagtaaagataaaaactttttttaaaaaaaggtatagaactgtcaatggcaagaagattcagagcatatgtgtcacctcccaagcctgacagtttgtttgatccttggtacccacatgatggaagaagataactgattcctacaagttgtttcctgatctccacatgcatgcatagcctttataagtaaatgtcagaaaaaataaatacaggcaaatacacttaacttgtgttcactgaaaatttctttttcagaaaccctccatagacaagtgcacaagatctttaagcaaaatagtagttctgtgctggcactttttttttcaagcccaagagcattaaaaccatgctatgtacattggtgttacagtaaaagcaagccaggagccactgaagctaataatgacaagcaagtccggaagagtttggacttaatgctttcttagtatcctggagcatccatctcacattccacagagatggctgctaccaacatctgtgctgtcaaggctctaattctgtgagtcttgaggacctctttggaaggtcatactttgctttctttctttttttttttgagacagggtttctctgtatagccctggctgtcctggaactcactttgaagaccaggctggcctagaactcagaaatccacctgcctctgcctcccgagtactgggattaaaggcatgcgccaccacacccggctaattgagttacttttaagatattagaataactggccaggcggagaagtagctcaagcgaagcagttatttctctccttcagctctgtactcactgttttcccagtgcccacatgtaaaattcagctcttctgcctcaataccactggggctgcctctgtgtggctttatttcctatgtcatttttctccccctcctgcactattgtggtctgttcttggaaatgtgtgtatccacttatccccgtgagcctctagaatgaaatttcctttgcacaactgctctcctgttgcttctttcccctgttgcttaacacctttgctcatgtctgacgctggactaatttgggaagcttcctaacctaccagtccatgctggatatgtgcaaatctttgacagtagggttgaagaaatgcagaggttctgtgtgtgttcccagcacccacatggcagctcacaaatgtttgtaactcctgtttaagtgagcattcttccttattacaagtaagtgacgctagtaataatgttttatgagttctgctttttctgaaatgctaaaggagctaggagaagaaaggattgctctcaagggtaaatcaagatcctcagcttttagctgcttgatggacatgagaatgtttacttcgtattttatttcagtttttgtacttacgaataaattgagtatttttggttgaggttctggcctcctcgtcttttttattccctatagacagttgctttagagcatcacttttggaaataggtgtcgtagctcatactgatactagcacttgagatacttcagggtggaggatcttagaggtcagtctgagcagcgtatctaatttcaagttaattcatgtaatttagcatcatcctatttttaaataaaagaaaacaaacctaaaaagccatgatttagtaataggaagagtttcttggttggttggttggttggtaggtttttttttttttgagacaaaaaactcactctagccttgtctgacttagaattcacaatgtaacccggttgggctccaactagcaatccatcttcttctgcttcttcagtgctaggataaagtcaagcaccaccatgccctacttttgtttttttgttgttttttgttttttgtttttttgagatagagtttcaatctatatagctctgttactttggaactcactgtttagccttgaaccaatgctgatcctcctgcatcagtttctgagtactgggattagagacatgggtcatcacgcctggtgaatgttgctggatttgtgtttttgttttctgggggataccagtcatcaagtctgtggtttcacaatgccagtcagatgctctaccactgagccagagccacagttacctctcttctctccccacacaatttctgagctaggtctctgtaatttgctagccttgaacatgatatctttctcctttagcctacaagttgttgggtacaggcctgggccaccaggatgggcaaggcgaaagttctcgatttatagaaagaaagaaaaacaaaatgctgcagttgctaaaatctatattaaggccaaatcttcagtctgaggaattggaaggataaagaaatttgctggttttgctgctgtggtgtgtgtaagtgtgctaatatggaaaaggacttaaattgcagagctgagtattattatctggtgttctacattcactgggagctaggaatgaactcccacagataagtctcttggactattttacatatttatacacatctgtagattttttttcttttttttttcttagatattttcttcatttagatttcaaatgctatccccaaaacctcctatccccccccccccccccccccccgttccccaacacacccaactcccgcttcctggccctggcattcccctgtactggggcatatgatcttcaaaataccaagggcctctcctctaattgatggccgactaggctgtcctttgctatgcatgcaactagagacacagctacacagctctgggggtggggggtggggtactagttagttcatatcgttgttccttcaatagggttgcagacccctttagctccttgggtactttctctagctccttcattaggggcccatcccaatagatgactgtgagcatccacttctgtatttgccaggcattggcatagactcacaagagagctatgtcagggtcctgtcagcaaaatcttcctggcatatgcaatagtgtctgggtttggtggttatatatgggatggattcctgggtggggcagtctctggatgatctttccttccatcttagctcagaactttgtctctgtaactctttcttttcttttttttttttttttaaacctttaacttatttcttttttttttttaaattaggtattttcctcgattacatttccaatgctatcccaaaagtcccccatacccccccccatcccctacccatccactccccctttttggccctggtgtttccctgtactggggcatataaagtttgcaagtccaaagggcctctctttccagcgatggctgactaggccatcttttgatacatatgtagctagagacaagagctctggggtactggttagttcatattgttgttccacatatagggttgcagttccctttagctccttgggtactttcttgggggccgtgtgatctgtaactctttccatgggtattttgtcccccattgtagagttatttttaaaattatctttgtgatgtatagtgtgatgacatactattaatttttacttttgctgatgtatagagtcctaaattcttgtaatgaatatgtattaatttattaaaggtgcaaatttctttttctctccaataaaatcacagataaaaacccaaagtaaggctaggtatagtgtggtgcatgcctctaatcccagcccttaggaggcagaggctattagatttctcagtttcataggaggcagagcaacataaatgagaccctatctcaaaaaacaagagtaagattctttggccacttggtagcctggttctcctgagattactggtacaccctcatattggaacagggtctcaagtagcccaaactgacttctaactacttaactgagactggctttgaatatataatcctgttgcctctactccaaagtgttgcaaccacaaacttattaccaccatgctcagaatattttgtattttaaaggtaagcattggttattaaaacaaaacactagtaagctaggtatggtgtgtatgcatgtaatcccagaatttagaagactgagattaggtttgccatgaatttgaggccagcaagggctacaaagtaagaccctatttaaaaacaaacaaacaaacaaaaaaaaaacaaaatccaaacaaagtaaaaccctgcaggagatcttagaatttatttttctacattgtatcaacacagtaaggttttttgttatgtgtgtagtaagtacagcctgttcatgccatacgtgtatagagggtcacctaaaacttctgggagtagttttctcctcctcagcaagctcctgcgatcaaactgaggtcatcaatttgtgtggtgctagcccttttatctgctgagccatctcaacagcctgagacttaagatttttttatacatacatacttttaaaattaaaaggttgtttcaatatgtaacccaacagaaagattttaactaaaaacatttgcttatctcaattccaataactgcactgaaataaattccttcctccctcaaatttgtatttagaccaggtatcacttacttcaagttaattataaaagctatctccagaatatatgtacaggtacacacacacaccacttacttcaaagtaattttgaattatccccaaaacatacatgtacacaccattaacatctgtctgtctatctatccattcatccatccatccatccccccacccatccatccacacacgtaaaaatagtgcttggtcttgtattcagatctcacctaccaccaccatctagttttttttttttttttttgagataagctcttttttgtttgtttgtttgttttttgtttttcgagacagggtttatttgtatagccctggctatcctggaactcactttgtagaccaggctagcctcgaactcagaaatccacctgcctctgtctccctagtgctgggattaaaggtgtgtcccagcacgcctggcttgtggctgtttctttaatgatcgtcttataacaaattgttttaaggattttataaatatctacttaggaggatcttctcaattctgtgatctgtgtaaaagaattattttatggtctggtttgttgcagttaaaaaaaccctgaaaatatggtagtgactgtaatgtagacttcagtgatttcttcagcataagtgtgccatcgggtaccattcttcacctagtaatgtagaagcatagttatggcttctttctgctgcagacggggaaacttttagcaccagaatgcacatcagttgtttttgcaacatggccttggtggtagctcagctgttggccttccttgtctctttttgagaaacactttgtatttcattaactatactaaaagatcggatacatgcagggaagtggggtatctgggggcctgccaggctgtgccgttaaaaattacgccatgtaacaggtttattgggagagtagggagactgaaatgcctccctagactgggacattagagagagatagacagacagacagacacacacacacacacacacacacacacaaagatagataatgaaggagagagaggagtaattgaagagagaaacaagagagtgatctggggtggggtggagttaggagggggttacacactgtgtgcacctgccacacctgtccccctgttggctggggtggaaggtgacataagccacctttatattcctgagggctaacactaaatgctcataaaagttacatcttgggtattgctcccccccccccccaaaaaaaaaaagaaaagaaaagaaagctgtacagtgtcagatggtagtttgtgatgagttttggttatacactgtcttagggtttttgttactgtgatgaaacaccatgaccaaaaccaagttagggaggaaagagtttatttgccttacacttcctgtttgaaggaagtcaaaccggtcaggaaactggaggcaggagctgatgtagaggccatggaggggtgctacttattggcttgctccttaaggctgctcaggaccaccaatccaggggcaacaccacccaccatgagctggaccctctcctattgatcactcattaagaaaatgtttacagttgaatcttatggaacacttggggttccttcctatcagctaactgtagcttgtgtcaagttgtcataaaactagccagcacattctcattatttagacatctgatttagctgcataggaaggaaaataaagcagtaattgacaataggtttgtggtttttgtttggtgagagttctgaagctagggctgctgttgtgactccctagtgccactgatggtagcctttaatttctgtgtagctttttttttttttttttttttttaagcctagtgatcacaggaggttcctgcctactcccatcccttactcctggtggtcaaaacagaagagagggcaagagtttgctgaggcagtttttctttattattgaggtttcatttttgaggcaggatctcgtcttaagtgttgcccttggccaagcatggtggcacacgcctttaatcccagtactcgggaggcagaggcaggaggatttctgagttcgaggccagcctggtctacaaagtgagttccaggacagccagggctatacagaggaaccctgtctcaaaaaacaaaaaaacaaaacaaaacaaaacaaaaaaaaaaaagtgttgctcttgatcactattcttgggttcaggtggtcttcccacctcagccgaggagctgctactgcccagcagcttatccagtgtgtgcaggccactcagctggcacagatagcaggataaacctttctctacaaccaagcccagtgctgctgcttattccattggccaccttttgtcacaagccttttaaaatttgagacagggtcttattccctatgtaggtgaggctgctttcatagagatccacctggctgtgcctccctagtgctgggattgaagactgtgcaccacccatgcctggctcatagacatcaagctcttgtaaactagaaaatcaaattagaaaccttcagttaggaagaagggaaagtgggattggctgttggtgtgtgtgtctctggaactggtctttataaagcattgggatttcttatcttacactggccagagttaaaaacagaaaacaagtggaagattgaaggtctgaaagaataaattcactaagggtttttttgtattcttagctaacaaaagaaagtaagtatggctctatttttttagggctgaatatttggattggggtgtatatatggcattgttttgtaactcttaagtccttattgctgtactagctcctgccatcttttatttatctaataatgcatcttaattaattctttgagaacttgtatcttgatcatattaatatgcccctcccctgctttttccctgaaactcttgccaggtccatccacttcttatctccccatcacattatgccttttttatttaacctttttatggatttattacttttaattttatgtgtgttttgccttcatgtatatttgtgtcatgtgagtttagtgcctacagcggtaagaagagggggtcagatcccctggaactgaaattatacatgttagccaccatgtgggtgctgggaattgaatctgtgtactctgaaagaacaatcagtgttcttgactgctgagccatctctctggcccctatgtttagtattttaagagatgcctggagagatggcttagctcttaagagcactggctgcggagctggagagatggctcagtggttaagagcaccaactgctcttccgaaggtcctgagttcaaatcccagcaaccacatggtggctcacaaccatctatagtgtggtctgatgccctcttctgggatgcagatgcacatgcaaatagagcacttgtatacataaaataaataaatcttggggggaaatccttaagaagagaccgtctagctgggtggtggtggtgcaggtggtgcattaattcaatgagaggcagagacaggtgagtctctgagttcgaagttagcctggtctgcagattgttgcagtatcctgtcttctgactcaatctttgttctttcttcgtagatttttccctaaacccctaggtatgtaggaagtatgttgtggatttagtggctggctaacccctggttagctccctgcattttgacaagttgtgaatggtttctgtgttacatgaagtttctttaataagcagcctgtcctacacttatctatggatataagatgaatagtaggagtgggattgagagctatactttgttggtttttgtttgttttgtttctctgtataacagaggcctagctgtcttgtagaccatactggccttgaattcaaagaaatacacctgcctctgactcccaagcactggggttaaaggtgtgtgccaccaggcctagcccttttttcccaccaacatcagtaattattatagtgtatacatagaattttgtctagactgaaaaataagaactagcaactaatagacaatcactgaggaagataggattatacaacagaacagttgaagattcttgtacaggtttcatggagggaacctctgctgaccagtaaatttcctttccttgtctgtctgtccgcctgttcgttcgtctgttcatccatccagctatccatccatccatctattgagtctggcctagaactctacctctagctcttgagtgttgagattaaagacatgtgccaccacacccagccaccagtagttttttaaaaatctcatttataattgcattagagttaataagattactcagatgaaactgttagcatattatttttgaagaaaaataaattaaaatatgagctgtaacacaaaagaggtgaataaatagaaacaaagcaacttttatttccatatatttagactttactatttagcattagtaactaaaatttaattatcttgatgataaatgaaattttgaaatagaattacactttgaaagaacaagtgtaactggtgaatgtaaaaattaataaatggcatggatcatttctgttagcattgtgaggtttgatatgatataaacatttctttgtagagtacttaaagtaagtgcttaatgttgtcctatattgcctgggagaatatatgttgctgtataaaatttaagagggatattaaatatatatttttgttattttaaggaaattcgaaatgaatcccatgactatcctcatagagtggccaagtttccagaaaacagaaaccgaaacagatacagagatgtaagcccatgtaagtactgtaagatacagagatgtaagcccatgtaagtactgtgagatacagagatgtaagcccatgtaagtactgtgggtttatgtgcatgtgtgtgttcttcagcttgtttcaggagacccgatgcagtgacagtctttgggtaaagattgattagtatagcaatttttaaacttttatatacttttgttttgagacaggctttcactctgtagtcccactgttctggaaattaccatgtagcacaggctgacctcaaatttgcagcagtcgtcttgtctcagcctccagaatattgggattattaagtgtcagccaccacacgaggtttaatgtatacattttctcatgtgtttgtacacacgcatgtggaaggttgaggttgatggtcagatgtcttcctgaattgctctttaccctagagatctgttccagctagtctggctggccatcttgttctgttagaacttgtcctgggattacagtcaggctgccacacccacctggccttccatgcgggttctagggatccagtctccaccccttacatttacttagtcatctccccatccctctggtttgaggaacatctgattagctcttctaagctctggtagaattcagcacttaattgtctgttgcactcaggtggaggatgttcctataacagctcagcatttcctatcctcatccagggttctgcagtctgccagtgaactgaccctcacaaaggctctccttccctgttgtacagcttttttctccaggtgtcagagccacttactgtgtgtcgtgtcatccccctcttcagcacattcatcctggtgtcctgatatctgagtaaacactatgaaacaccagccttagagtacctaggccttttcagcactctttgatcttgtgcccacaaagaaagcagggagggattttttaaaattttgtggcagggctccctgcatctgctatactgtcttccaactgtgacttcctgatcagtctcctaagtgctggaattacaggcctgtgcaccacacccagctttcaccctgcaccttgaaggtcactttctgtgtgtcactactgcttttgttgtttatttttttgttttgctttgttttatttttgtttgtttttgaatatttcacttagaatgtatatggtttttcctggcattgggttctttaatactttgagttttagattactcaagtgttgctatatccttcagtttgtttgttcgttgagacagggtttttctatatagccctggttgtcctggaactcactctgtagaccaggctggcctcacactcagaaatccaccttcctctgcctccaagtgctgggattaaaagcgtgcaccaccactgcccagctgctatatccttccttaaccagtctgtttctcatatctatttagctgttttattttaaaatatctttgaccttaattctccaatttatgaagccccattttggaagactttattctttattcccttagagagtttaaaaactcaagaacttagagtcaagaaacaacacttaagctgtaatgtgacaccggggaagtgcctcagcttatgcacatccctgaagtcccagggcttctgtttctctattcctttgtacatacatcccttccttctttcctcctcctggtctgccttgctatcttcagcctctttgtccctgcttttccttcttattacataaataggatgtagaatgcttttccctttccctcccacccccaagttatttcctaggtagatctatgcatctacacaacaggggtttttgtttttgtttttgtttttgttttttgagacaggatttctttgtgtagccttgactatcctggaactcactctgtagactagactggcctcaagctcacagatatccacctgcctctgcctccaagtgctgggattaaaggcatataccaccatcgctgctatcatcactgccctgctgaacataacagtttaaaaagaatatattctgggactggagaaatggcttagaggttaggagcactggctactcttttagaggacacgggtttaattcctagcacccaaatgcatctgtaactatagttctagggaatctgatgtcctctcctagctagcctctgtaggcactgggcacacaagtgtaggtacataggtatacatgtaggcaaaacacccatatacataaaatttaaaaaaaaaaacaaacccaaaacaaacaaaaaggccaggtggtggtggcatacacacaatcttgactgggggtgggggggggtgggaagaaagaaacaaaaagatcagttagttctcctttctgtctgtcctgatcacctgtgctctcttcatgaacccccatgcagctaccacaccctcctcactggaacttagttaccttgctagctttcagatgcaatcaatgactttcccttggtttcccttcttatccttctgtgtcttgtgagattgccgactctgtcttccttctcaaggacttatttactttttctgtctgctctcccatttttagagccgtccatcctcaatttatctttatttttttcaagggtcttttcctctcctcactattttttcttaatctatgagtgatctcattttcttgggggccaattgccatgcttattttcatgacttcacatttgatctgcagtcttggtcttaactccaaacagatgtttgcaagttgtcatgtgccaacctgacatgaagttagtcaaagaaccatagaaccatagaaagcttatagaaccaatggttgggtattccctacattgctttattccttctatttgtaactggtaccaccatttacatattgactaatactggaaacagactccttattcatctgccacatctaattagtaactaaatagtttactgtgagtgtgatctatttaaaatgttttcttatgtacatactagcttttttgtgatttattatagtaacttctaaacttttgggactatatagatggctcaggagttaagggccaaatactgcttttgtagaggacttgggttcgtttcttagtatccatctggtggctcacaaaacacctgtaacttcaacacatccaacaggcatgcagccccccttctctctctctctctctctctctctctctctctttctctctctctctctctctctcacacacacacacacacacacacacacactaaaataacctctatagctccattctattactgctttcttcctttctcttactatccaagttgttttcccgccccattaaggcagaaatttaagtttgtcatcattatcattctagactttttatatacctttatgaccctttatacacatctagattttacttatagctattttcctacaaattattcatttgagtaagagtggattacttagcattctcccattatgactagtttctaacatactgtgttctcaaatatggtgctcactcatttctttcttctctttaatgacagtgacggtcttctccctgcagtgccaaatcagccgcgccttccgccggctggtcttttcaccatggagaggatattatggaagcagggttttgtgtgtgagcatgcattttaatgttttttgagacagggtcttctgtctattatgtagctctggctgtcctggaacttactctttatacctggctggcctctaactcacagagatctgcctgtatctgctgtcaggagatagagacagtggagtcactgtgagtgtgaggccaacctgatctacatagtgagttccaaacctgctaagaccatatacaaagaccctgcttcataaaaacaaataaataaaagctgcacagatttatgattctattagtatggcttacctataatcatagtccattgtagctacagaacaagaatattctcagtaaatatgtgtataaatgagttgatacatggagagaaaataacaaaaagttagagacttatcttcctcctccagtgaaatgacattgtgctcttttggagagagggtctcagtatgtagccttggctggcctgaagttttacatacatcaggctggctttgaattcatagacatcaaccttcccggcctcccctttgctaggattaaaggtatatattaccacatctggggacaagtctgtgctcataggcttttaaaatttatttatttttgtattttatgtatatgaaggtttcttctgcatgtaattctgtaccacatgtgtatgtggtaactgaggaggctaggagaatgtggatccctgggacttgttatccaagtaacagatggctgtgagctgccatgtgggttctggaaattaaaactagtcttccggaagatcaggaaataattcttttttgttttgttttgttttgttttgttttgttttgtttttttcaagacagggtttctctatgtagtcttggctgccatggaactcactttgtagaccaggctggcctcaaaactcagaaatccacctgcctctgcctccaagtgctgggattaaaggtgtgcgccaccaccgcccatcagcagataattcttaactgcataagcatgatgtctccagcaatggacctttttttttcttttcctccaattttttattagatattttcttcatttatatttcaaatgctatctggaaagtatcccataccccctcccccactcccctacccacccactcccacttcttggcgctggaattcccctgtactgaggcacataaagtttgcaggaccaaggggcctctcttcccaatgatggccgactaggccatcttctgatacatatgcagctagagacacgagctctggggtactggttagttcataatgttgttccacctacagggttgcagaccccttcagctccttgggtactttctctaactcctccattgggggctctgtgttccatccaatagttgactgtgagcatccacttctgtatttgccaggcattggcatagcctcacacgagacagctatgtcagggtcctgtcaacaaaggactttttttgtgataaatttcatactgtaccctaagctgacctcagttgtggcaactcttcctgcctcagcttcccaaatgctggggttacagttgtaagccaccacagctagccttatttttggctttttgagaaaagggtctccccaagttggccttgatcctcctccttctgcctccaaagtgctaagaccctgtgtgctcatggtggacacagtattataaagaagttgatttatctcttgcctatcactgaatctcatgaggagcagaagtgtgaatttattcaagataaggcttcttacttaaccctcctatgaaggtattgctgctggaagtgtactaaacattaattctgcattgccagaggtgctgaagattgcccttatcctagcaaaatggattgacacagtagtagcatgcctatccttatgtgtgactcatccacctgtgatgtgtgtcaggtttgctgagcacagaatgagcctcattaggggtgatgattagaagctagggaagtgagtaagcctctgcaggaccaaccaaaagggaaaaacttggctaagtgctaacttgtactcgtgtcatgattaaggatgataccaaagaattctcagattagcgatgtcattgatgaacacttcaaatgcaagaaatgaaaaatagaagcttttgggggagagatggctcagtgtgtaagagtgtgtgctcttgcagaggacctgggctcaattcccagcatttacaggttgctcataaactgtctcttaactctagttccaggggatctgatgccctcttctgatatccattggtaccagacacacacatggcatacataacatacatgcagtcaaaatagtcatacacctaaaaataagatccatatgtttataaaaagttttttattttccttatcaaaatgaaactttaaaaatactgatgaaattttagtttagtgttttaagttctcaagtcattgtttgttttgttttgttttaaagacagggtttctctgtatagtcctggctgtcttagaactatctctgtagactaggctggcctcgaactcagaaatccgcctgcctctgcctcccgagtgctgggattaaaggtgtgtgccaccacgcccagcttggattctagttttatagacagtgaactgttcccactacgttctttactgtgtttattcatgcagagtgcagattccttgcaggtactttctagagaacatttctaagtacatggaaagcttcaacagagacctggaaacagctggtttatgcttttttgttttgggggattgaagcaaagactaggtgcatgttagacaaatgctaaccatgctcttacacctctatccctctgtactatttctcttttgagacagcgtactgctcagttgcataggctggctcttgagccagcatgtaggcctaggcaagaaacaggtcttaacctttagataatcttgcctcaacctcctaagtgactgggaagtacaggtctacctcattgtatctcaccgttatttgcctccatttgtttcaaaaacccctctcccgtgccccctttatctcttgattctttatcaagtgcagccttcctcctcacccttcccagactgaagtttccattccccagtaaggagctgtttgggttctgttttgtttgtttgggtgccaggtgtcaaatactgggctgatgacttctaagttggtttcatttcctagctgttgtgaatagttcatagtagtatacatggatgtgcaactgtctgtggcatgttggcatagatttctttgcccacgcttttggaaagttgtgggaatcttttcttgttcttttggtatttgttaactatttcattaaaaatttatacattagcttccattttttttcattcatagctagggtattccaattttcttttgctatttatggatagtagaataaaaaagggagtaattgccgggcgcagtggcgcacacctttaataccatcactcgggaggcagaggcaggcggatttctgagtttgtggccagcctggtctacaaagtgagttccaggacagccagggctccacagagaaaccctgtctcgaaaaaccaaaaaaaaaaaaaaaggggggggggagtaattgaaaacactattacactgccatgatattatgcctttaatcccagtactcaggaggaagaggcaggtagatcttttgagttcagggctctgtgagttcaaagctagcctggtctacagcaggttccaggacagccagagatagacacaaagagaccttgtctcaaaaaccaaccaagcaaaaaaacccttactatacttacacttctcactcactgaataattcaatctgacagcccttggggtgggggtgatggtcttaaaagtcatgtaaaggtgataatgtcctaaaaggaaagaccttgtgtggcaattgagctaagagtgagccctgtattgttagattaggattgctgtgttcttgtatgttaggacctggttttcagtgagtactgatatgtagaaaaggctgggaatgtatagttcagaggttgagtgctttcccagttataaagacatgtttgaattttggcagccatattcatatgtttattagccttttttattgagacttttattttttaacatttttacttttatatatatgtgcatgcatttgtgttgggatggccgggcatgtgtacatatgggtaagcatctttggagccagagggttgatgcatctgaggctagagttataggctgtggtaagctgcccaatgtggatactgggaatcaaattcatgttccctgcaagaggacatgttcttaaccactgagccatgtctccaacataattgagaagtcttaagagcaattatgttctcttgtcaccgggcatacttagctttagttgttcaagttcattctttacaacaaggaaacatggttccttggatgagtggctgatctctgggaatggaacaatatgtaatcccttaggtagatgtagtgacaaaagcttgtgttcccagcactcaggagtcaaaagcagaattgtgagctgaacagcagcttgaacaacactgccagattctggctcatagaaaaggggctggatatgatggcacatgcctttaatcacagctctaaggagacagacgggcagatgtcaaaggctagccagagctacagagtaagaccctgtctccaaaacaaaacaaataaaaggaagaaagaagggggagtttagaatgtctcaaatttctagaaagtacagattttattaccgattgaataaataaatacttaaatacaaaggggagatagtaattgtccagaagaaatgaacaggaaaaaatttagtattgaaggctgttgaagatgtcttgggaagacaccaaaagcataagttatatacagtttctaaaattagtgaattgtaatagatgtttgtgtgtgtctcctacttataattctagtacttgggagaccagggcaggagaaggctgtgagtcctagattcatctgggcttcagagagaaagactttgttcagaaaaataaagctgagttactgaatggtaatgtgtgctttcaattcaactcttgggcagctagggcaggggaatgctatgagttcgcagccagcctgattacacagggagttccagacaagctgcaaatacatagtgagacctcctcttaaaataaaaggcataaatgaacattttaaagttttggagaataagatcaacagattgactagggaatatatatttgaaataggcagactagaaaatggacagaaggtatgaatacaaaaggcagataaatgtgaacgtgaaaaacatcattagctattagaaatattagtatgtacttactaaacagagggcatactacctcagggctgcagaggagcttgatagaggctaatcaagctgatagcaatggtgtagctgtatccccaaaatctcccaagtcaggggttctctttgtaggctaagctggccttgaactcagaggtttgcctgcctctgcctcccaatagctgggattaaaagcatatggcaccatacggtgatacgactatttttcacaaacctgaatttacacttctgtgacctagcaaaatagattttttagcatcttagagaaatgaaaatcctattcttacacagctgtgtaagtgaatgttcataacagctttatttggaatttaagctcaagtgatcttaaattgggaacaacccaaatgcctttcagtaataggtgattagtaaatttatattgttggatattgctaaacaaaccattgatgcacacacacacaaacacacacacttgggtagatctcaaagaatttagattgagagtatcaggagattacataactatatttttatatataatttaaaagcttttattattgcctgtggatttatatatgtcatggcacaacaatggaagtcaaaggacaaattttgggacttggttctgtaaactcaggctgtcaggcttccatggtgaacactttaaatagtagctatttaccagcctctatttaacttttttttttttttttttttttttttttttttttaaagatttatttatttattatatgtaagtacactgtagctgtcctcagacactccagaagagggcgtcagatcttgttacagatggttgtgagccaccatgtggttgctgggatttgaactctggaccttcggaagagcagtcgggtgctcttacccactgagccatctcaccagcccaacttttttttcaaggcagggtttctctgtgtagccctaaaactcactctgtagacctgactggcatcaaactcggagatccacctgtctgtgcctctgcagtgctgggattaaaggcctgtgtccagctgacttttttttttcttcttctagatggttggttgtcctagaacttgctgtgtgatcaccagcctggctcatagagattgacctgactctgtccttcaagtgctaggactataggtgtgtgctactgcgcccagcctatgccattcctgaagtaatacagtgtcagtagtggagactgtgtttagtgatgcagttagaggtaggggtgagaggaatgggactcagtgtgaaatagcacacagtacctttggtgatgtaatagttctatactacaccatagttacatacatatataaagaatacacagaaatgaatgcctgtaaaaatgggagaattccagtaaggtgtgaattggaccaatatctttccatggttttttaatttaatagcccgttgaagttggtcctgggtagcataatccaggcgaggtgggagtcagtgaggaacataatggttctgattaccaagagcttcatcttaaaaggcaaatcataatgagttcagggcctgagggaatgttccttccttgctaagtgactgagaatcttgtcctacggtagcattttgagcaggcccgtgtagaagccgcagttcattgtctggaagctctgctggagaacgtgtactggatggtgcagccacagtgcacgtggagagaactctgagtggggatgcatggggtgccctccagaactacctgtatttagaacttgggggaagatgaatgaagcagaagtttcaaactactaaaatcttttagataaaataggaatgtcagagaatttgtagttgtcattggaatgtttaaggctgagtctttctgagtagtcaagccgctccatctgtcgtttccttcatatgctgttttcttttgcaacactctgcttaacatggggtgtgattgcaagtatgtggcctctaagagaggaacctagtattacattgaacctagtattaaattaattggacatgattttgtttaatacaaacacatagactgcaacaattgtgggtttaagacagagtctcacattggttaggagttgctgaggctgggatgattttgaacttgtggtatttttgctgggatgacaggcctatctgttaccatcctggttaggagggactaggaagttaaagctctggtgttctaggccagcactctatcttttgagctacatcctaagcactaggactctagctcagaataatggaattgccttatttataggccctagtgaggtcaaagtcctccttagtcaatacatggaattagggtgctaatgattgaacatgaccttatggaaactcttatctagaacttccttattcctcttctaagtctgatcacacagtgtgattggggttgttcagtagtctgtagctgtgaactgttctgtgtgattgaaatccttcagtttaagtccctctagtctgaaaagaatgatgactttctacctgatggcagcaaaaagggcatgatagtttaccatctgctttataaggttatgaggattgaggcggtgaccgcctagtaagatgcgtctggcatatcataatcactaaacagtgctttctgctagttcaagttgattgtatggtcagtgagaagctatttataggtttgtttgttttttttttaaattgtaagtaagcaggtgaagtgttcagacttccatatgtgctaagatttatatttcttcattaaaatgatagtggttatgggacttagatttagttctgtctttgtgtaggactccttgctcagtttatgaaaaaaattatatttattttatcagtttttggagttgaacagtgttgacatagttataagatgtgctgagttcttatcaggaaagtaccccaagtagagattaggaaaatgtaaggaggaaaaataaccagtaatgctatcttaatagaattgtcctgtatttgctcaacttgtgagttcataattggcttctgccatgatgaacatgaaccttgccctctgtttgacatgtgacactctgcagtagcagaggcgtttacgacagtgatcaatgggggaatatcctctgctcttgttcttttcagtttattgtgaatgttgaaagaggaagcttctgtgtgcgttagccacaggctgagcaagtcatttcttgagagctgagacaatttcccagaagacactgagagtgggaggcagagagacttcgtcagaagtgggtgggcataatttttaatccaatgttttagttacagtattgttagatattaaatttcataattcactaattcactcaatgataacaattaagaacaaataatatcctacagaataataaactgtgttccttacagtgctgctgctgctggtactgttagaagtggagaagtgtgattcctgtgaaaaagtggtttcttaccttccccttcctttaccttttaaaagatgggtgggcaggggctcaggaaggtgcgggagaatatgaatgagccacagaaggctgagcaagtactgtgagctcaggtgcctttggtgatctcactttagagaggctggagggaaaaggtggaagagcacaaagcatgtgattttctgacctttttaaattgtaactgtggagtcaaatgtttccttgttagaaataaacaagtcagggctccaaagagcaggaagggaaaggactcaagttcctggcatctacatctgtcagcttacagggaagccctttttggcctttaaaagcacccacacactcatggctcacacacccacatacatatatgtagtgaaaaaaataaatctttaaaagaaaattcaagccgggcgtggtggcgcacgcctttaatcccagcactcgggaggcagaggcaggaggatttctgagttcgaggccagcctggtctacaaagtgagttccaggacagccagggctacacagagaaaccctgtctcgaaaaaccaaaaaaaaaaataataattcaaacagttcacagatttgggatcagctaacactactggaccagggaaaggaagcagttaactcccgggttgggagggaacctgcatgtttattcagactgagcatgtgtaagtgattcctgcctgcttagtgaatgccagggtcctttgtttttgttgagaattggaagttgtgaagtcacagtttcgcaaggttttggtgggaatttcacttcctgaaatgtatatatgtgtgctctttgttagatatttcagggtggtgtatattaattaacaccaaaaactgtctatggttttctttttagaatctcttttgaataaaagtttgtgtgtgtgtgttgatacagggtcttatgtatcaaaggcctggaattcactatttagaccaggctggcctagacatagacactgacagtgttaacagtgtgtgcaccatgctctgttctcacagtctttttcatagaactaaataatgacattgctgtgtatctgagagtttgatttcctgataagaacatataaattggggctggtgagatggctcagtgggtaagagcacccgactgctcttccgaaggtcaggagttcaaatcccagcaaccacatggtggctcacaaccatccgtaacaagatctgactccctcttctggagtgtctgaagacagctacagtgtacttacatataatcaataaataaatcttttaaaaaaaaaaaaagaacatataaatttagtcaccagctgtgcttaattctaaaaacctattatgttttcctaaagattacttactgttttctattttcgtctgccaacaaaactttaaaacttgttttctaaaccccatagaatgcagttttcctttgctgacttgataacttgttaggaagtgatgcaaagccctgtagttgcacaggaccttgggtgactttcacgtagtagccagtgcactgcactgtgcttttagtcattgggtatgattactgcaaagttttgggttaaagactgaaagaacatatctgaattccaggaacagagaccactgcttaagctgctagcttttttctagtcttagcgtctaaaccctgccttggtctgggaaggcattatgtaggcaaactgaattcctagcaccctgtgtggggccttaacatcttgaggtgtgttggtcaaaagactgtatgatttgtgtgtgttttggtttgtattcttcctgtgattgcaacatgagggaatgcatccctttgcaggatactgttgtggaatttacatttttgtggttgtattgctttctctaagagcttacattcttctgttggtgtgctatccaggaaacataatggtctttattctgttaagtaggaagctgtccagtatcttgtacagtgtgtcaattataagatgactaagctgttaagaaaacaacaattaaaaaaaaactttttaaaaagtgcttgctgacatcatctttccccaaagactaattaatatatgtgtttactatagcaagatcatagttgttctgttgtcaagtgacatgtagtatttacagaggccttctgttcagtccaacaggagcagctgagatagattcatacggaaactaaagatatttaacatttttattcatatgaagaatagtgtctttgggtcttattgctgtggagacacatcatgaccatggcaaggcaactcttataatggaaaatgttgaattggggcagccttacagtttcagaggcttagtctgttatcatcatggtgggaaagaaacatggtggcatgcaggcagacttggtgctggtgaagaagctgagagttctacatcttgatccatagcagcagaaggtgactgtatgccatactgattgtcaccttcctccaacaaggccacacctactgcagccttctaatagggccaatccctgtgggcctagggggaccagttaccttcagactatcataaatagtttagacagtttggggttttgttttgttatagttgtctggattttggtttaaaagtggttgctcaaaagctagttaaaatagcacacacctttaatcccagcattccaggaggcagagtcaggcagacctatgagtttgaggccagcttggtctaccacaaaaaggtgtggggaagagggagaaagaaaaaaaaaagggagtgagtggtgaggttaggagtagcctaaactggggagaggtagtggtaagaagattaggattttaagatcattctttgctgcatattcagttgtgggccagcctgggctacatgaggccctagcttaaaaaagaaaaagccaagagacagtggaaagagagtgactatttagtatagatttctttgaaaggagtagtttatcacaaactatattctctgaagttgaacattatacattctgttagtaacttagtttatgcttgtgtctttttttttttatgtttacgattcttaattaaagttcatcttggggataagtgcttacatgctcctcatctttttttttttctcatctaaatgatacaagatgttgtattttagatgttgaaattagcataaattctaaatccgaaaattttatttttaaaatgtagaaatgtgtctaacctgttaacataatcgtaagtaatgaagtgtttccagtactcttgtaaagaatacagtttgggtgatccagttattttagcttttccgtgttaagaattatttaaatatacttgtatgcatgctaccgggaaatgataagtctgttctcagtttgagcccagttatggttttgcagttatggttttcctcagtccctgggcactgagacttgagatggcgtttcacttctgggaaggtaagtgtcttcttcactttactgcactttactgctgggcagtgtctcaggttactgtagatagagtgcaaacagaagcactttttttccaacacccagtaacaggattatagtctcacttccttattctaaaaatagaaataggtaatttaggacatgtctcaactctcctttggtattaatttatgttttctttatttctcctatcatctataactaccttaaaatattaaaatcaaataaaaagtatgaaaagttgaaatgacagttatgatttatcattctctactttcagatgatcacagtcgtgttaaactgcaaagtactgaaaatgattatattaatgccagcttagttgacatagaagaggcacaaagaagttacatcttaacacaggcaagtggtatggtgctgagcagaggtggaggggctgctgtgtttcagacacggctaaacatgggtctggacatctgcaaatgacatgctatcttttagttttctgattctgaaatcagttttgcaaatgccatagttttcagatttcgaatatattgataaaaagtaaaattgttcctaatgttactattttcaatggagggaagttttttgaaatgtcttggtgcagttctcttcaggtaaattgactgtatctaacatggcttggagttcttgtcccctaacttgtgatgaagaactagagaggccctggaagcccttttctatccttctctcccctatgaatatactttcagagagtatagtcatggatgctgaggatagggtctgaccatctgcagagcctagacatttgtacactgctgctctgctgccttttctcctccccagtggctcctcacagcaacctccacagtgcaggaactaggaacttctgagatcatagcttactctttatccagtaagaggctaaacagcaaccagcactgtcacatttcccagtcaaagatgttcagaaatgtaacttaaaatatttgagagttttaaattgtatttgattattatgtgtctctgtgtgtatatttatatatacatgtgcacagggtgtccacagaggacaaaaagaagtggcccttagcactgaaattacaggctgttgtgagctgtacaacatgggtgctaggaatcgaactctggacctctggaagagaagaaattgtccttaactctatctatagtaccatgtttgaggtttttaaaaagccaagaggttaatgcaacctgctatttgaaggactgagaaaagtgtgaagtaattgtttttttaagttcagcctaatgtacctgtgagccattagaattttctctgctacagaaacattgtttcacttgaagtttcctatgactaaaaggaaggagaaacagtcaggtgtaaaccttgttaaggatattaattcacaattcataggacaaaagaaagctagagaatagggctggagagatggctcaaggtttaagagcactgattgctcttccagaggtcctgagttcaattcccaggaaccacatggtgactcataaccatctgtaatgagatctggtgccctcttctggtgtgtctgaagacagctatagtgtactcatataaataaaaataaataaaaaatttttaaaagaaagctagagaatatacatttgcaaagatgctatgtttgtacatagcataaagtgggcccagtcactgagcctccaccaaggactcagctgctttcccctcaggccgcctctctttcctctcctcagatcttgtcctgcccttgccccctaaccttccttctatagtcactttctgaaggtagtttacacctaggagaaggagtgtttgtgagtcctttgagaacttttactctgttgactcctttgctcatccaaataagagtgatgagttgcttcctgctaggcacacacttgagatcaacagaattagcaggaatatcccaaccgccactctgggattgctagtgtccatgcagctaggtgtagcattatgaaatagatttgcctgcagtggtaattcagctatactgagactaattcctgaagagacaatagtagttcattgcagggagagtatataatactcagtggtcacaatgtgcttgagaaactgacctgagaaattctacatgtgtttattccaacctcagtttcacaagagggctggcatgggttgaatgatgaatcatacacataccttttagtagccgtctttaatgcgaatggggagcataaactttaatttgctgttttatagtgagtgaaatttatcttatgtatagtcataatcatttaggatttcaaaaaaattcaagaaatctatattcagaactgaaaatggactttgtactgagaatgagtgtgggcatgtggaaaggcatgtgtgaagttaggctctcgttgctatgacaaatacggaaaggaaaactgaaaaggaaaaatcatttatttggcctcaaatttccagagatatgactcaaggtcagctttaaggctaaggtgaggcagaatattgtggcagtgtggacgaagagagaaagagctacatgacaagggcattcctgtgacctgcttccccctcttaggtaccacctcctagtcctggtacactaatctaccaacagattaacctttgattaggccagagccctcccaagcatccatgagatgacatcaggcctgcagcactcgagccccaggtggagcctttcccacctaaaccgtaatacagctattccttctcctgccctttccctccagatcattgttcttctttttccttttactcagttcctacagacaggtctgtctgtctttttttttcttttttttttttcctcttttttggttctccaagacagggtttctctgtgtaactctagctgtcttggaacttgttctgtagatcagaccggcctccaactcagagatctgcctgcctctgtttctctgagtccagagattaaagtcataggccgccacctgggctaatgattgttttataagcatctatatctcatatgaaaaaattcctgatgaacaattccatggtttttaactcagttatccctgttggactgttctcatgaatcctgatagacaggactctacacactaggagtgtgagaatatagcagcttgtctctagatttttttgtttatttgtttgtttgtttttttgttgttgttgctttggtttttgtttttttcctgatttcctttctgtattctggtgtacttttcccagttttctaccctggccttatgagttgctcagttccacacacatcagtgcagcattctaagatgtttccttgcaaccagctcttcagacatggttcgctactgtgatactcccacatctaccatgtgccctggttctttgtgtgcctcaccttctgcttaggacatgcagagtgaccataactacccatgtcctgcctatcacctctctgccagacttgccttcctttggctagactgtaaatcctcaggttttcttacaacataaggcctgaggattgagcatttggtggtagtgatgctcctctgtgcaccttgtagggctgagactacatctcagttgtacttgtgcatagcatgttacttggtcacagagaaaacctgcaacatttagtgtttgttaactacagtggtagtccttgttgcttattaattagattagctttctttcccctgagttttaggatttcttttcctaaccaaccccttggtatatcatgtccattgtacagtttaaaaaattggtagcattgctagtttttgaaagaactgatacacgaaaaaattcttgtcttcttaaaatcctttattctttatactgtaacagctgttgcatgtttattggttagtctttaaaagcagtattagaaatgatgtattcatgtgcacacagaggacttcactcctgagttgcttcatgattttttttaaagttttatttatttattttaatacagatggttgtgagccttcatgtggttgttgggaattgtttttaggacctctgctcgctccggtcaactctgctcaactctgcttgctcagtccctgctcactctggcccaaagatttactacactgtgtacacagtacactgtagctgtcttcagatgcacaagaagagggcatcagatcccattacggatggttgtgagccaccatgtggttgctgggatttgaactcaggacctttggaagaggagtcagtgctcttttccactgagccatctcgtcagccctcatgatttttttttaaagtgtactttattttcatatctgtatataaattcccaatgaggccagaagagagagatctggagctagagatatgagtaactgtgagccacaccgtggtgtcctttgaaagagcagcaagtgcccctaacaaccaagccatctttctagtcctgcttcatgattgttgtttttgagacaagatctcactgtgtagccctaagtggcctagaactcactgagtagacaggctggccttgagctacagagatcctcactcttctgggattaaaagagctcaactgggcccatgcgccaggcgtggtggtgcacgcctttaattccagcactcgggaggcagaggcaggcagatttctgagttcaagaccagcctggtctacaaagtgagttccaggacagccagggctacacagagaaaccctgtctcgggaaaaaaacaaaaaaacaaaacaaaacaaaaaagcctaaaaaacccaactgggcccatgattttttttttaattttttttcttaaaaagaactaattattttattcaatacgagtacactgtagctgtcttcagacacaccggaagagggcatcagatctcattacagatggttgtgatccaccatgtggttgctgggatttgaactcaggatctctggaagagcagtcagtgctcttaaccacagagccatctctccagcctccccataatttttttttaagatgaaagaaaaaggcatttatgttactgggggttttctcagtgttctttttggtagagaagatgatgttggttcacctcactgtatgctttatattgtaagtagatagtgtacattggatggagagaatagtatcctttaatgtaagggggtcgtttctcagattactagctttttacaggtcaacccgagagtttatccatctaagtgattggctagacaccaagaaataatctgccctaaagatgatttaccttctaattggggtggggtgtagctctggtattgtattctctgccacatgacactggtagttggttacatgttacagttactttaggcacagtggaacttagttctatttcagccttcactcagaattattaaggattctgtcttagtcagggtttctattcctgcacagacatcatgaccaagaagcaagttggggaggaaagggtttattcagcttacacttccacattgctgttcatcaccaaaggaaatcaggactggaactcacacaggcaggaacctgggggcaggagctgatgcagaggccatggaagtgagctgcttactgccttgctcagcttgctttcttagaaacccaggaccaccagctcagggatgacaccacccacaatgggccctcccaaccttgatcactaattgagaaaatgccttacagctggatctcatggaggctcctttctctgtgataacttcagctgtgtcaagttaacaaacaaaaccagccagtgcagatgaaggtcggttgttagagtatctacctagcttgtgtgaggccctccgttacatctccagccccacataaaggaggaaaggaagtgacggttggacattctttttcatctcatggcttggtttttttgtttttgtttttgttttttttttggggggggtggtggaggttgtttttgtttttggagacagggtttctctgtgtagccctggctgccctggaactcactctgtagtccaggctggcctcgaactcagaaatccacctgcccctgcttcccaagtgctggggattaaaggcgtgcgccaccatgcccggttcatctcatgttttttattttttgttttttatttttttttgttttttcgagacagggtttttctgtagccctggctgtcctggaactcactctgtagaccaggctggcctcgaactcagaaatccgcctgcctctgcctcccaagtgctgggattaaaggcgtgcgccaccacgcccggctctctcatgttttttaaaaaacccctatgctatcattaatcgaaatgtaaaagtaattggaagtaatatatttgatttttcctcctccattgcattttgtaatatgaactattattcacttttaattctgccagatgatactaggcataacaaaccaatatgcaggtactttcatggttatttttttttttcctgcaaattcttttgtattgacttggggttgttggttggttagtaatgaaataaacttccctattttccacagttccctgatgtttgtgtgagatatcacctgcctgttacagaaactgaaagagacagatttccttttctaacagcctttttagctagaatacgaacagccaggtggtgttatactgtaggcacctttggtaataacagcaaagccatatgtaataagacagtgaactgtgtaataataactatataataagactaatgaacaaggctggctctgtttcggacagaaatcttagctgtattttatagttgttatttgaacctagttatccagctcttcccataagccctatatggttcttttcctgccaaggtaaagaaaatgtctttacatggaggtaaagttttattgaatgcacactatattttggatttaagtgcatttgaagtcttaatttactcatcaggccccaatgcactggttataacagtgtgttccagtagcatccttgcagccttgtccttccccagcccctctctcatgtggtcttctgtcgttacagggcccacttccgaacacatgctgccatttctggctcatggtgtggcagcaaaagaccaaagcagttgtcatgctaaaccgaactgtagaaaaagaatcggtgagtattcttctcttctcttctcttctcttctctcctctcctctcctctcctctcctctcctctcctctcctctcctcttctcttcttctatcttgtttgtgtgtttgttttgttttgtttttttggagacagggtttctcagtataaccagccctggctgtcttcactcactgtgtagaccaggctggcctcaaagtcacagagctctgcttgtctctgcctccttagtgctaggatccaaggcgtgctccaccatgcctggctggtgagtcatattcaacattagtggttgaatatgccgcgtgtgtgatgtgtgatcgttatcttacacatttcttttattttggtgaatgcttgtggtggctaacagctagtggccaggttatttctttctttttttttttttggtttttcgagacagggtttctcttttatagccctggctgtcctggaactcactttgtagaccaggctggcctcgaactcagaaatccacctgcctctgcctcccgagtgctgggattaaaggcgtgcaccaccaccgcccggcagccaggttatttctgaccaggactaatggatactttgctttcctttcatttctttacagttttttaaaaatgcattttatagatttatagtaatacatatcttattctgctacataagcacattttttataagtataggtgatatgttgggagtattccctactttcagccctcctttctcccactctcctattatcttttttgtactccccgacagttctactactgcttttgtgtctgtctcctagattttgtataggtaggtaccttaaagcacacacacacacacacacacacacacacacaccacttctctttgtgagattgccatatttcatttggtacatttatcttagttgcatctatagttctgcaaatgacataactttgctgtttttggttaaaagttttcattctgtaaatataccacattttcttcatttttctgctgttaggcaagtgtattggattctcaatttaggttttgtgagtagtgccttggtaaatgtcaataaagtataaatctctctgtgatagacctaccttggacttggagtcctgtggttagatactcagaagtgatatagctgggtcatattgtaggtctatttcacttttgtgtagaacctctgtacagatttccatagctgctggaccagtttacattttgaatagcagtgtgtagcggctcccttttggtcacatctttgccagtgttttgggtttggttttggtttgtttagtggtagtggtgtcactgttgaggttttcctttattttgtgtagccctggctgtcctggaactccctgtgtaggccagactggcatcacagaaacctacctccttctgcctcctgagtgcccgaattaaaggtgtggattgctgtgtccaacagttctttcttttctacagtcatcctgactagagggagatggaaatgtctgtgcggtttcaatttgcatttctctgatagccagctagactgaaaactttttatgtatgtaatgacatttgtgtctcattttgtctgttcattagcccatttactgattgggttgtttgtctttttgtttaattttttcagttctttatatattctagctattaactgtctgtcagataaattaggtagcaaagatttttctcctatttggtagattctgttcatttaactacttgcttttctatgcagaaacttcttaatttcatgaggtttcttttgtcatctgtttgcatatttttcttaagtgagtagagtcctgttaaaagtcattgcctgggctggggagatggctcagcagttatgagcactgactgctctccagaaggtcctgagttcaattcccagcaaccacatggtggctcacaaccatctgtaatgagatcagacgccctcttctcgtgtgtctgaagacatctacagtgtacttagatatataataaacaaatcttaaaaaaaaaagaaaaagtcattgcctagggtgtctgtctgtatgcgtgtgcccacgctagggggagcagagtttattcttcagtgctttctgctttatattttgagaccttcttgggtcctgttttgactggactggttgtgagccctcaggatctgcttttatgaacccacccactcctccccagtgacatctgcatcagaaaccacatggatgaagctgccttggcttctctctgggttctctttctcttgggttggtttgtggacctgctgcttttgttactatgactgtgtggtatgccttgaaatcatgtctgattagacctctagtctttatgtttgttttcttttctcaggcttgcattgcccttccccccaggcctttagagcttttgtaagagcagtgctgggatcaaaggtgtgtaccaccactgccaggctaagcgctttaagatttttctgagatgtactttcaaatagcattggaattttgatggggattgcactgaatctgtagattgctttggatagtatagccggttttacaatattaaccagtctacatacttttcttcctttcttctttctgtttgtcaatattggggactggacctcagcctttatgcctgctaagcaagaattttcccagctaagccatagttcctgtgggcagagaaagtcttcagggttttgtctccccccccccctattgttgtagcttttgtagagatttttctttacctaggtttattacatggtattccttaatgcagttagaatgagcttctatccttcatgcgttgtgagcatgtttgttattgagataatagaaagacttctgattttatatattatatattaatttctatcttgatgcaaagacttctcattaatcctgaattttcttttgcagtctttaggatcttttaaaatggaatgattttatctgtaaagaaggatacataggcgtcttcctttcctgtttgtacctcactcattttcttctcttgctttacttatgattgagacttcaagtgtcatgttgaataagggtagagaaagatgagaacttttgtctttctcctgatttagccaacatgtttcagatttttctttggtgtgatgctggcttttgggttgtcatttatagcctttattgtgttgaaatatgttcttcctattcctagcctccttaggacttttattatgaagggatgttgttttaaatatcacgtatgcaatttctccttgagtcattttacgtagtgaattatgattggtgatttgcatatgttgaaccacccttgtatctctgggatgaagccaacttgatcatgatgaatgatctcttatgtcttgaattccattactgatacttattatagtgagaactttgcagttttgttatttgtgtgttggtctttgtccgacattattattagagtgatgctggcttcataaagtgaatttgctagcatttgttttgctgattcttagtattgtttctttagtttccatttcattcacttctgcgcaggtgttaattcttaccatcccctactttgggatgtgcgcgcatgtccagtttccatagctagcctatctgagatctctctgatttatttacttacttgacacttagagctataaacattcttttgagaactgctttgtatccagagagtttggaatgttgtgcaattctatatatttttctgtttccagcggctcatatttctttcagtggtgtgttttcttttttgtttcctttttgcttggtgtgttgttttcagtctgtaaatctgcatggtttctgtagtttctcctgttgttttgtagttttgttcctttgattttctataagattgtgtgtgaatgtttcctgaggcacacatctgctagctccaggtagggcatcagtgatagaaacaaagtgtagtttggcaagctaatgaatggataggtgtgagattatttttagaaacattgggaattaagtggctctactactaccaaagaaaaacaactctctgtgagactagtaatttttggaggcattgtacggccttggctttttatatttcctttgtttctcttttgtatatatgtatctgaggttaggctgttttttgttttttttgtttgtttgtttgcttgttttcgaaataagtttgtagtgttcagtaagatctgggttgtagtagtgttgaagtgtttttatctgttgggctctttttcagaacaccagactcaatatccagtaactcttgacagaagtattacacctttataaacaatcactaattaagagtaaacccattataaatatatagaaataatcaagtagcactcagtcgccacttacaccccaagcatttcagaaagtaaaggaagccttatagaatcaagccttgtttactgagctgctagttactgtgagtgtgactacaagaaaaataccagagtgcctcacagttggtattaggttaatagagaaaatctcgaggatgagattagatgagagtcagcaaggccaagtagaagggtgagtcagttttgcctaaaggtaggatggaaagggagagctaaagaaatggtttcaggggttaagagcaaacactgctcttgcaggagcccctagttcagttcccacacctatgtggtagctcacaaccatgttatttcagcttcagggggactcagtgtcctttctgagttcaccagacactgtattcacatgcacaccccctgcaccacacacacaaacacacacacacacacacacacacacacacacacacacacacacgctttaaagtatgtttttaaaaagaaaaagggccaggtgcagtggtgcatgttttttatctcttcattcaggaaacagaagcaggaagatccctgtgagttccaggccagataagtaagaccatgtcttaaaagaaaggaggagggctggagagatggctcagtggttaagagttccgacagctcttccagaggacctgagttcaaatcccagacaaccacatggtggctcacaaccattaaatcagacgccctcttctggggtgtctgaagatagctacagtgtacttacatatattaaattaataaaaagaaagaaagaaggaagaggagtggaggatgttatcagaaaacccaatatctataatattttgccagttcaaattataactaagatttatgtgtctttagactctttttctgtcaaaattagaaagtcttgactcttaagattatttccagtttaaatataccacacacacacacactcacatgctttaaagtatgtttttaaatctgttctatgacagatttctggttcttaattcttcctaaattaatagttactgggttttgatgataatgtgtatcatgacattggcctaacagaatttccaataattgccatctgtagttgttttccgtcctctcccactaatctagagattcaggggtggggcatagttttgtttgtttgaatgtgttgcaaactagatgatttgtaaatttcatccctctcacgtcaaagtagattcactttttttaagattcatttcttttatgtgtattattttgcctgaatgtatatatgtgcaccacataagtacctggtactcccagaggctaaaggaagacatcagatcctctggaactgggttaaggcatagtgtaagacaccatgtaggttctgagacctgtgtaggaggaacaagtactcttaactgctgagccattcctttagtcccttgtgggcattttaatctctggttcctttttttttttttttttttttttttgttgttgttgttggtgggggtggttttttgagacagggtttctctgtatactcctggctgttctagaactcactctgtagaccaggctggccttgaactcagaaatccgcctgcctctgcctcccgagtgctgggattaaaggtgtgcgccaccatgcctggcccttaatctctggtttcttaagtgtcagtgaaagtgcatctgctcttttagtacttaaacgtcattttggcagggtataaaaaacttagctcttgcttttacttgaatttaaatacattcttttcattttagtgttttagattactgtccaagtcaaatggatgtcatttttttttcttgtacctatattacgctggggttttctccctttttatttatttttttaaattttatgtttatgaatggtttgattgcatatatatatgctaccatgtgtgtgcctgttggatcccctagaacaaggtttatgggtggttttgtcccatcatatgggtgctgggactcaaacccaggtcttttgcaagaacaagaagtactcttaacaactaagccgtctccctacctagccaggctctctttctctttaacattaacttttgaaactgtatcttaccacttgattggctttttgcaagtaaatagttttgtcctttaaatagttgcaaatgcttttttttgtgtgtgaaaaataaaaagattattaaattatatttgggatttgttctgttcctgtttgtcattgctggtgtttttgaggggtgtgtgtgtgtattttctttgtatttgtcctgctcttgttaatcctgtttctatttctctctatttactgtaaaatgtccttcttgtcctcttaagctttatccattacacttatcctgcactacagttctaggtgtcaaaacaaaccaaaaggcttattcttctgattggctgtcctggaactcgctctgtagaccaggctagctttgaacctagaggaaagccgcctgcctctgccaaacaagtgctgggattaaaggtgtgcgccaccaccgttcagggtgacagaaagcctctttagtgctctctgaaataacattcttgcaagagcttgatacttctagaaactcaaactgaatatgttaaagtgtaattatgctttaagaacagcacacttttgggttgggcatggtaacacatgcctttaaatccagcatgtgggggctgaagccagcttgatgttcatactgaattccaggacagccaaggctatataaagagattctgtctcaaaataaagaagagcatattttaatgaatattttatgtttatggctttttaaatggaaaggaaacattttatggagctactttacagaatattctcattaaaatacttaagatattcttaggtttcagtacttggagaagtatagtatgtatgtgtgagatttgtttttatggttttataatatgtctaaactattcttaaatttaggttaaatgtgcacagtactggccaacggatgacagagaaatggtgtttaaggaaacgggattcagtgtgaagctcttatctgaagatgtaaaatcatattatacagtacatctactacagttagaaaatatcaatgtaaggcttttcttgtttcaaagtttcagttttatctgatggtcttaagaattcttgggggttgatactatacttacttgcctttggatatttgtaataatatctaggtataagcatgtttttagagtatttctatataaagtaactctttatgcattctgaagaattctaaaaacaacaaagaattacaatgttcaggttaatattaatatttaagaattgaaaattaactattggagctggaaggctggctgtagtgttcttgtagaaggcccaggttcagttcccagtacccatgtcacgaggcttaccactgcctgtaactcctgctctgggaaatccttgtataagaaaaatgctagaactaacttctccttgatttttacatttcagactggtgaaaccagaaccatatctcacttccattataccacctggccagattttggggttccagagtcaccagcttcatttctaaacttcttgtttaaagttagagaatctggttgtttgacccctgaccatggacctgcagtgatccattgcagtgcgggcatcgggcgctctggcaccttctctcttgtagatacctgtcttgttctggtaagtgtcatgcgatctcattggtgttttgtgcttctctgttttgttttatcattttataatgttgatgataaaaagataaataattttgcatgactggtgcctgaggaggttagaagaaagcattgagtccctgaaactggagttgcagatagtaagaccctataactcgtgggtgggaaccaaaccctgatcccctttaagagcagccagtgctcttaaccactctgcctgttaatttgtttttaactgtttctataggcagcagtaaattttgaagtaatatcccaaaactctagggttttttttttttaaacactttacttctatagtttaaattgactctagctcctgcaggtacatgtgtgtccatgacaattataaagattccctagaaagtctcaaccaggcttggctcgtacctttcatcccaacactaagtaagttggggcagaaaatttgctccaagtttgaggttagcctgggctacggagttagaccctatccaaaataaaaaaggaagaaatgaagaatacagtaaagtctccctcctaaaagggagaaatgacaaaggaaagcaatcctgaaagagtgactcatgcctgttgtctgcacgtctgagccagccggggttacctagtgagattcacctcaaaaaagaagagaatgccaacgaactggggccatggcaagcatgagcactgactcctagtgcatgggtaaaagatgagcagagtagcacatgtctgtgaccccagaacgtggtgaagggcagttaggtgggtttaatatagtttactgaccaccagcttatccaagtcattagctccaggtccagtaaaaaacactgtctcaaaaatggcttgaattctaccctagaccctactgggtaggagaaaactacctccaataagttgttgactttaaagatttttaagccactgtgctggtacctgtctgtaaccccttgcttgatagaggcaagagaagcaagtccatagagagtttgagcaagtctgagcaacatgagaatttgtttcaataccccctttctttctggtactttgtatacagaattaaaagcttctaccttcagcaataaatttttaaatttaaaaatgtgattttttttccttcttggataagtacatcatctcttatttataaaccaaggctaaaaagtacttcccagggttactgtgataattctgtatattaatgatattttctgtcctggttaacgtcagctaacaacttgacacagcccagagtcatctgagaggagtgcctcaattaaagaaagactttttaagatcagattggtgttatttgtagggaactaattgatacaggacagcccagcccactgtgagattatctgtagagagaatgtcttgtatattttatggatacaccacccatcaattaaaaagcctatggcttaggcaggaagtaggaggtgggacatcggggaggcagaaagagttctgggatagagccaggcacgggaggatttgcccaggaagatgtgaagagatggatgtggggtacctgaacacaggtaaccagccgcgtgtcagaatgtaaattaaaataaatgggttatgggctggagaggtggctcagcgggtaagagcactgactgctctttcgaaggtcctgagtttaaatcccagaaaccacatggtggctcacaaccagccataatgagatctgatgccctcttctggtgtatctgaagacagctactttattgttattattattgttgttgttgttgttattagatataataaaaaataaatctttgggacggagcgagcagaggttctgaattcaattcccagcaaccacatgatgtttcataaccatctgtacagctacagtgtgctcacatacattaaataaataattttttttttttttttttttttttttttggtttttcgagacagagtttctctgtgtagccctggctatcctggaactcactttgtagaccaggctggccttgaactcagaaatctgcctgcctctgccccctaagtgctgggattaaaggcatgtgccaccatgcctggcaataaataaatctttaaaaaaataaataaattattatgcttaaacaaaccctaatgggctactttaacattttgcctagacaagttatagtttcaatcaaaacttgaattgttagagctgagaggtggctcagtgatgaaggacagttgctgctctttagaaaactaatgttcggttctagcacccacagtgggtgactcacagccctctccagctccagggatcacatgctgtcttctggccgctgtgtgcatctctacacacttggcatgcacacatacacatgcactcacaaatctttaaaaaacaaaaaactcatttactaataaaattaaccttttgcttaaaggcagtagattctgttaatgatcaatattaaaactgggttatgagaaccggcggtctttggcagagtgtttgcctagcatttaagggttcatgagtttcaaactgcattggatgttcatacctgctctctaccaactgtgccttgctcatccttccttctcttcagtttatggtttgtttctaaaaccagatcagtagtccagagctcagaggtaattttgtaggtcaaggctttcctatttcctgccatcttttaagtgtctgctgtacatccacaaagcatgtctttgtccagatttgagatggggaagggcactgagcagcaaactttatctctgacaaaaccattttttaaaactatataaataatcctaatcatatctgggtgtctgtgtgcatatttctcacccccccccccatgtgtgtgtgttaaattatccttcttttaagaaggaacctgttcacagctaaggtctgagcagtgagtggccttatattgctttttttttttttcaactcaaacaacttggttttacctttcttgcagatggaaaaaggagaggatgttaatgtgaaacaattattactgaatatgagaaagtatcgaatgggacttattcagacaccggaccaactcagattctcctacatggccataatagaaggagcaaagtacacaaaaggagattcaaatatacaggtaaactcagtatcattagtgtcaaataaccatgttccatctaaatcctcagccttcccctacttcatttggtagatccagctgactgtctagaattcataaaactttccacagttcaagaggcaaagatgaaagctagtcaccaagagttcaagaccagcctggtctatacagtgagatcctgacttaatttttttttttcttcaagatttatttattattatatctaagtacactgtagttgtcttcagatgcaccagaagagggcgtcagatctcattactaatggttgtgagccaccatgtggttgctgggatttgaactcaggacctttggaagagcagtcggtgctcttaaccactgagccatctctccagcccccaatttttttttttatatgtatataagacacatatatacatatatacttgtttcctcagtggaaacaagattcaattaaaataatttttttcatatgagaagatttcagcatttaattattgaaagaaaaaaaaacccagaaaacataagtacttttatcttagtaaaaacttactgtagaggcctgaggagatggctaagtggtcacaggttacaagcacttactgctcttccagagcacccaagtgtgtttttaagaactcatgtcgggcagctccaggggatccagcatctctggcctctcatggcacttacagtcatatgcacatacctaaatgcacacacacagaactaaaaatgaaggaaatagccgggtgtggtggtgcacgcctttaatcccagcacttgggaggcagaggcaggcggatttctgagttcgaggccagcctggtctacaaagtgagttccaggacaaccaaggctatacagagaaaccctgtctcgggggggaaaaaaaaggtggaaataaaaattttaaacccttcatgtgggagtaaagactatttctatataggtactttatagctgaataacttcagttcttactacagacataaagtgctaggagtaaactttacaaactttacatgtctgatggtgacattctcaggtggtgtcttttttttttttttggttttttgagacagggtttctctgtgtagccctggctgacctggaactcactctgtagaccaggctggcctgaactcagaaattcacctgcctctgcctccccagtgctgggattaaaggcgtgcgccaccacacccagctctcaggtggtttttattgtatatttatagaccgtaagctcaaagagtatcagaacttagtttcatgtgataaggaaaagtgtaaggtagaaagagaaataacagtatatccttccctgcatcgtctgcataataaagacatacctagatcgaaaccaacaatcttaatccactactctttgactatctatttataaatatctgctttaaaatagaatatatcttttagggggctagagagatggctcagcagttaagagcattgactgctcttcctaaggtcttgagttcaaatcacagtaatcacattgtggctcacaaccatccgtaatgagatctgatggcctcttctgatgtgtctgaggacagctacagtgtacttaaatataataaataaatttaaataaatagaatacatcttttaaatcaattctatgtgattattttaaatgtctatgaatgtgtttgtgtttgtgggcaagtgctgggataaaagttaatgtcaagtttacctgtctgttccctgcctcatattgagtaattgcaatttgtagattgtgatttaaagaaaacaaattttattgttttttatttctgttctatacctagaaacggtggaaagaactttctaaagaagatttatctcctatttgtgatcattcacagaacagagtgatggttgagaagtacaatgggaagagaataggttcagaagatgaaaagttaacagggcttccttctaaggtgcaggatactgtggaggagagcagtgagaggtatgtgccatatatgtgctagcctaccgagatatgcgggtagtcactacactcagggagtctgcgacaggaccaccctgtgttcagacagttaatgctccttttacttgtgtcagagtgtttaggtggtatgtgatgacataaatatcttcagagagttttccgtcatcataagaaagccggtacagggcggggcagtggtggtgcatgcctttaatcccagttggtggcttactaccaacttaactccagcttttttgggatccaaggtccttatggtggtttgagtgtgcttagtccatgggaagtggcactattattggccttgaagtggctttttttttttttttaagtttatttattgtatgtatgtgagtacactgtagctgcacagatagttgttgggaattgaacaagggctcactctggtcagccctgctccctctggtcaactctgcacgcttgctcagtccctgcttgcacctctaatggcacatacctaaatggacacacatagaactaaaaatgttggaaataaaatgttaaaacccttcacgtgggagtaaagactatttacagtgtacataagtacactgtagctgacttcagacacgccagaaaaggtcatcagatctcattacaggtggttgtgagccaccatgttgttgctgggatttgaactcagggccttctgaagagcagtcagtgctcttcccgctgagccatctcgccagccctggagtggctttgttggaggaaatcaatcattgtggggatgggctttttgcggtcttgtgctcgagccccacccatggttgaagagattaggctccaggctgcctggagaagacagtcttctggtggcctttggttcaagatgtagagctctcagctcctccagcaccatgtctgcctgcccactgccatgcttcccatcatgaggagaatggactgaacctctgaaactgtgagccagccccagttacatgttttgtttttataagagttgccatgatcatggtgtctcctcacaacaatagaaaccctaagacaagcctccttaggcttttggttttttatttttatttagatttatttaatttacatgtatgagtttttggctgcatgtacaccacatgtgtgcctgatacacacagaggccagaagagggtgtcaggtcctttaaactgatcattcagacagtgaactatcaagtgaatactgggaattttccctggctcatttgcaagaacaagtgttcttaaccactgatccatttctccagcccaagaaatcaaaatttgaaaacatagtaatgataaatttaaaagctattgtttagagacaacaaaaactccaatactgctcatatacagaagttagtgtaattaggtgataaaaataaatagtataatacagaagatgagttgtataaaaatacctaattggccgggcagtggtggtgcatgcctttaatcccagcacatgggaggcagagacaggtggatatctgagtttgaggccagcctggtttacagagtgagttccaggacagccagggccacacagaaaaaccctgtctcaaaaaaacaaaacaaaacaaaaatgccggccagcaaatagaagcctaatgacctaagtttgttcactgagtcccactgtagaaggaagatacccaacttctcacagtgtcctctgacctctacaggtttatacctctctttctcctcctccctctccctccctccccacctctcatgcactcaagcagagtaattaaataaaaagagaaattcagagctgtatgcctatccccatgcacttaggaggcagaagcaggagaatctcaagttcaaggatagcttacatactgcgatcctgtctcaaagcaaatcattaatcctatctatatctagtcaaggctcatgcttggatctgaatgagacctgatgccttggtgttcttagtcagaacagatcccccacttttttctcctctgtttgaaagatgatcttttagcatgccttatagtttggatttctctaataattgttcttcttggttagaatcaagttaaatgttttgactagaaaaggtcttgtgatctagtagacattgaagagccagtcaagattttttttttaaatcaagctgcatgttcaaagctgtgacttaaaaaatgactgccaagccgggcatagtggcgcacgcctttaatcccagcacttgggaggcagaggcaggtggatttctgagtttgaggctagcctggtctacagagtgagttccaggacagctagggctacacagagaaaccctgtctggaaaaaccaaaaaaaaaaaaaaaaaaaaagccaagactgaagcactcacagtgagtgaaaagagatgggttttgtgagatttttttttttctagaggaagagagtaaatggctgacatgttgacattggccatttttcaatggatgtcagagaactttcattgaaacaaaattctatgttggacaggaaaatgggtgctagatgtacggatggtcaaatagaagactgggtaacctccaaacacagcattcatagaaattgtcaacagtatttgtcaaaggacattgaattttcatatgctgtcacgcaacaggaccagatgctgagaatctccagtctgtctccttcgtggtagagaggaaagcagagtgtgcagaaaacatgtctgtcttagcttcaggtcctgtaaaagcgttacttactgtaggcacagcagacgtgaccctgaactgtggagcacaccactggcctgtggagccgcttgtccttccctttgttactgcagtacactgaatgagatggaaagcatacaaatgagtgtccatggtttgatggtggcctgggccccactgtcagtgtgatgaaggcctgcagtgcagacactcacttgtttctgtctattcacagcattctacggaaacgtattcgagaggatagaaaggctacgacggctcagaaggtgcagcagatgaaacagaggctaaatgaaactgaacgaaaaagaaaaaggtggttatattggcaacctattctcactaagatggggtttgtgtcagtcattttggttggcgctttggttggctggacactgctttttcactaaatgttctataaattaatagttttacccagcacctttctgcactagtagctgaccgtggtgcattaatctcaagggtttgttagcaatgcctcatacccagaaacactgcgctagagtagacatcagccagataagggatattacagtcacaagcccagcatctcaggactcatcactgcaggttcctctgagacccagactgtcaatggctcacaataaagacaagcatgcttgttggatactgttacttcttacagctgcgttcacaccagtgtattgagaaatcctttatcccaaggattggcttttggaggccttctggattttaacctgcacttgatataagcaataaacattgtggtttttttctacattattaatggaaagaaaatatcctttaaacaatgtatgtaatatgtaatgtactgttgaaatgggcattacaactttatataaccattttagggtaaatatattttataagtacctatttaatcttacttttgtagttaaatgtactttttaaaggttcaatctgaaagtctgttatcatagaaaaataaattgtatgttgactcagttgtatactgaatacattttccctttcctaagcagacgtttgatagaggcagttgaaactataagcaagctaagactactacacattcttatttcctttctatttatgctttatcttattttaaaaagaaaaacaaaaattttctaaacatgtcattgaaggaaattgtttttttctgcgatagttaagaagtgacagttggtcaaaatatagttgaaaacaaacaaaaacttggtttctgcaggatgtggtagcacacacagtgctcaggaagctaaaacaagaggctcaatggtttgaagccagccaaaactacatagcaaggtcctatctttaaagataagagaaaaatagaggtggtggaggagagatcagacaacaccaagaataagaaatcgattcttagccatatttaatggacaaacctgtcatctcagcttttgggagatagaggcagaaggctcacaagttcaaggccagcttcaactacatagctagccccagagtttggggccagtcaggactgcaagaaacactgtctcagaaactgaagtggtttaaaaacattttgatttctgtaaagtaaagcccatgcatgactacactgttaattttttgtgaaaatgtaaatgtaattacccagacgggataaattatggttagtaagttaaaggaaccagtgttttatacttttgatttcagttcactagactgaaattttgaagtaaaaaaaaatttaatttctttacaagttcaataaatagtacaatggtgtacaaacttacattgtcccttacctttgtaatgagtatttttaaagcataaccactaattgggttttggtggtttcaaaccctgcttggtggaaaggttccaaaccattaggacagcattgctgcttcatctcttttatatatcacgtaaaagtgcgtggtaaatcttaattagtttaaatgagacagttaatttcttaatgcagtttgaaccccataggtgtagttagaaattgtgaatggccttgaaaagcatctcacaaagcgtatgatgtatgtgtgtgtcctgactcagcatagctgtcctaaggctttgaaatggagagcaggtaagaaggatgtttcctcttgtctgtttaatctctgtttaagcggaggccttagaattagatggctatgggttttgagctttctaacacttactggtttgtttttccaaaatgtagtatgttatcctactagaccttattaaaacttacagtccaagccaataaggtggcgtacacctttaatctcaacactaagaacaccaagacagacagatccctgtgagttcaaggctagtctggtccacataataagttcccaggcagccagaaatagacattgagatcctgtcttgaaagaaagcaaaccaactgaagatagcctgagcttaaacaacttcccacaagaaaaactgataaggctgagaccagtccttccttggacgatatgctttctagagatagcattgagcaccactctttctgcctcttggtgtgtattttatgtttgtgaggattcctttggcatacggaaccctcagtgctcctccccggagcccgtctttctcccctgaacacatctttaaggatgagttttaacaggagaacctttaagtcacactgtcatgttgcttactaaaggtacatggcctgtggtgacagtgtcactggcatcatcctgagcctgtatgagatgtgctgtgctgatgagagaagggtgctgggcagagaagggatactagcagtttctgatgggttcacggctttaaacacagtgtgcgtcagtctcggtagcagcttattttaactaatttaggaataatagtttgtcttggatcaaattctgttttttgtttgtttgtttgttttttgtttttggtgtttggtttttttttaatttggggaaaaaataggctttttaaaggggattattgtttactggaaagaatcctcacttcctgtttcctcccaccttgctgtaatgtcagtggtcacaagattcaccaggtactgtgttatctcagcctcctgatttctatccatgctcaaacctaaagtgtaaaagtacacattcctttttaaaaatacgcatatgcatcatttctacgttcagcagaatctacacatttgtcaagttttccacagttctcagttctttttatccattccgttatgtgtcacctcatgtatcaaacagtgaacataaaaagatatgaagacctgtattaattagtttttgtccaaacagctgtgctctgaagctgcgtcagaggaaaggtcctaatttctgagctcagcttccatgcactcggctcggccctttgtcttaaagtaaagctagtgctgtgagtttagaactgtggcccacgtttcaagttatgacacagaacagccctctctggttgtcatttcatttccttgtttgcttttagcaccagtcccagggtgctggctcccattttctgccaggcacagaaaggctacagctgactgctttaaaaatagctctgcgtagattctgcagagaagctggaacctaatggtagtaaaagtacttttttttggccattgtatacaatctacttaacaagtttacatttctgtcaagacattgcagactgaagatctacattgccttaatttgttacttactgatacaaatctttatttgtagttgttgttttggataggtttgtatattcttttttttttttttttttttttttgtatgtgtgttgagatagtaccttgccattgcccaagcctggccttaaactcagctcaaacgactttcctacctcagcctgttgagtaactaacaccacaggtacacactgtgcacacagctttcaagtataaatcttaaagagattattttaaaactgtagataagatttcaggcccttagtcaagcgtggtgcataccttctctgagtagggccatctctgggtcctggtgagtagtgtctatgtctgtgggaaggaagggctgctcggggccttcatctggctgagctcgattcatctgttcatagcatgggacaaaataccaacagaaatgtccattctatttacatgccaacacctaacaaagtctcttatttttaaaactcctttatatggctttgccatagaatcttgtatatacttttttttttttttcaaaatagaaatgattttttttctcattaaatttgtcatcttattacttgaaacgtgggcctttgttattggcagtggcttgctcccgaggaggcctgttctgtccaccctgtcccagaacgcactcatttgagtcagatgccacagttcttcctcacactggtctttggtttataccatgcagcaccatacctagagtcacagctgtctctaattgtcccctgaatatggaatgagagactcaggctgtgccctcattcactgctgctctgcactggagcctgtccccaatcagagaacttgcctcgtggccagcagtcttccttcctgggtcctgagcagcttcaagccttctgcattagtgctttctcttagccgtggctgttgggaagaagacccactgttctccacaggttgggttgttttttttttttttcctggctgtccttgtcccagcacagtgccatcagccattgtgagcagtgcttaaagtggaaagctacaccagcctaagaggctttgtgtaagctgacgtttaggatttaaagagcctggaccatctgagttctgactctgaagctctgcttggttgtaaagttccagttgattctgagcagtgaggtgtgaggccactgtcaccggtagggtctgcttggatgccgcctgctttacttggatctgttttgttggggactgctgcaaggagaattgcatgggaattttcttctttttctttacagagacttataagcatcgagttattctttgtagtcactcattaggcatagttttttttttttaagacccatgatgctgttgctattccccccccccttttttttttggttttttgagacagggtttctctgtataactctggctgtcctggaactcactttgtagacctcaaactcagaaatccccctgcctttgcctcctgagtgctgggattaaaggcatgcgccaccacggcccggctgctgttgatattttaaatgactattttaaaaagtcgttcagtgtggaaagttgaggagaggaagcctaggtaagttcctttaaagcatgcttggctcacctcggttagtcctgatcaatctcagtcggatgctaatgtaaatgtcgtgtggcaaaacaacttttaatgcagtctgactttccctctaacacgggcaaggaagaagacaccagcatttgcctctgcagcacagaggcagcccccaggatacccacgtagctcattgcttggtttgctcgcccattttacttttgccttattaaaaataaaatggtgaagatccattcaagtgaatataatagaattatctcaaaagccatttatcttaatagtcttacaaataaagtcatttcttagaagctattccattgatttcctcttattttgctacccctaaacactatttgaaaagaagtaatgagtttcaaaaaccacagcgtgtctgttaaatggcaaatttattattcttggtaaatgtgtatttaacaaacactaggaaaggatatctcgtgtgtatgtgagagagaaagagagagtgcttcacaacactttaaataatgccagccatattttcagataagaaacccagtggaggtgtgactcacgccttattttccagcctgtgcagatagagctgagatgcagactccaggctgtggtttcagtccctccaaggctcaggctcattgtgctactccactgtgtatttacttaaaccagatgtttaagcggggaaatagtagacaccccactagtggaggggtggaatcccttttacaatgcttcactgactatggcggaccagaacgtttctgtgccaaagccccacttcattcctttctgttctgttccacattctgccagagtcagaaccagccgtttggtcccaggtcctgcgacccattgctatctaaagagtatggttccctaatgagaacactgcagagaatcactgttgggaaatcaaacaagactttgtagaccaccacaggggcttggtagatctgcctgcctatggagaaagaagccagtagacaggaagaagcttcattctcatggttggggaggagcctaagtggtggagatctagtgtattgcctgtttatacagtgataaagtcaagtattttcatgggtagagagcgagggtggaggaagggaggggctgcgatcggtgcaaaaatggaaatacctttaatctcccaaaagctttgaccactggcaaacaattgaaatatcagcaaagactactgctcttaatggtcacaccctcttgtttaaatggcgtccccctcccaagcattaaattgcgctgaactatcacagttttacttagttctagtagttataatcattagcattctccttcaggagaaaatctaaatgctggaaatctaattcagagataacaagccaactttatgtgcaaactttatatttaaactgtttctagcagtgttacagtgattgtccaaactggattagacttttgcgttgaaatcaaagtatgggtaagtctagcacatgtaataaaaccttgctgtttcttgtggctacatttttttttttaacttgtctgtctcttagcctaccatgtagaggtcatttcttgagttaagatgggatggcctaaaagattcagtgtgtagttactgaagaagtaagtcccggcgcctcagagcagtctgtctcacagccccgcttccatttggaaacctgccattctggaaggaagcacttggtgttcttggaatgttcatgttggaatgatttttgttgttgttgttgttgttgactttttagttcagtcttagttcttttgtgtttgtatctatctatgtacatctgtgtgtgtggtggccatggattgaatagatgacttcttattttatgttttaggccaagattgacagacacctaaatgttcatgacttgagactattctgcagctataaaatttgaacctttgatgtgcaaagcaagacctgaagcccactccggaaactaaagtgaggcttgctaaccctgtagattgcctcacaagttgtctgtttacaaagtaagctttccatccaggggatgaagaacgccaccagcagaagacttgcaaaccctttaatttgatgtattgttttttaacatgtgtatgaaatgtagaaagatgtaaaggaaataaattaggagcgactactttgtattgtactgccattcctaatgtatttttatactttttggcagcattaaatatttttattaaatagactatgttggtt

SEQ ID NO: 5 - DNA, 9860 bp, Homo sapiens
agtgcctcaccaccacctgcccgcgcgccgccccatataaaggcgccgccgtccttacccccaggactcggccccatggagacgccgccggccgccgctggcgcatggcggggtgaggcgcggcggggagggtgcgggcctcggcggggaagcggcagggagcgctccggcggggtccgagccgagccgaggggaggggacgggaggggacgggaggggtggagaggggaggggacggaagggtcggggtcctgttccccggcccgccgggcctcaggacccctccaactttgcccaagttgggagagccggggaagagcaccaggttcctgatcgggatcgcgggccgagcaagcgcgggtctcgggccgggacagcctcggcgtcaccagctcccggcagccaaccaaagttttccagcaacttttctccccacgggtcaccccagaaacttgccctccccggctccgggagtccggccatgggccttgcgctgcctgggcctgagctctgagtgccggacgggtgggcgaaagtggtggcctcagtggggaagccttgggccccgcaacgcacttccagccccaggattccatctgagagccccgtccctgccttctgggtcccctgggttggcgtgggggttcgaggctttacagctggggtagttccagtcttagttcagactaagttccgatcttcgagccaccagctaacagctggatgaccttgagcacgtttttaatgtctgtgcctcagtttctccctttatagaatgggaatgggaatgcctagctgggagagcttttgagaggacaacctgaactaataacacgatgcagtgtgtagtgccaccaatccctcatcctgctggatgtggttttgggagggaggttaggagagagcgctattcaccgtccctaaccctgttggttgttcagtaggagctgtggcgcggggccttccaggagtctgagctatgagtggcccctgtggagagaagcctgtcctggaagccagccccaccatgagtctgtgggaatttgaggacagccacagccgtcagggcaccccaaggccgggtcaagagctggccgctgaggaggcctcggccctggaactgcagatgaaggtggacttcttccggaagctgggctattcatccacggagatccacagcgtcctgcagaagctgggcgtccaggcagacaccaacacggtgctgggtgagctggtgaaacacgggacagccaccgagcgggagcgccagacctcaccggacccctgccctcagctccctctagtcccgcggggtggtggcacccctaaggctcccaacctggagcctccactcccagaagaggaaaaggagggcagcgacctgagaccagtggtcatcgatgggagcaacgtggccatgaggtaagtgtcacttctgtggccaggacacatgaggttcgcatctctcctgtggccaggacacatggaaggatgactgtctctgcagctctggtgggctggaagaagtgacttccttcttagggactggtctagagggagggaaagccttttatgaggctagggtcgcgctactggtaagtgacagaggcagggttccagcccaggtccctctgtccgcagagcttaccttatgctgtgggtccctccctgaggccagcactgtgtctccactcctctcctgtaatgtgaggcagggctgtaaaggggaccagcccgtgggcagctgctcactcacccctcggaacctgatctcaggtgggtgttgtgaggagggagtgtgcggatcccggagaggagcaagcgttcaggaaccatagtagtggtagtaggcagtggtagtagcagttgttgttgttatttcaggggctgggcttctgaggccttcttgtctcagacctgggctcccacgaaaacaggtccagtgactgtgtccaagagtgacctcaaggctgggtccagctttacatgggttgtgggcaggtggcctgatgctctggcctcaggcgagctgcaacttggatccctactgactcagctgaagcagcctgggctggaccagtctctggaggttccccggggccagagccaggactcagcccccagtgactcccacccccatcccccagcacaggaatggggtggggaggtaggggcagagagggaagccagggctgctgctgaccttgggaaacccagacttctcccgtcaatccaggctccaccctctggccccacttttttcccagttccctccaaggcaggctgatgagtcagtgctgggtggatggctcacagggaaaagttcctgttggcatggacggggagaccccagacacagggttgagaagggatgattttctggctgccctgagggagctcctaggtgggctgcccaaggctggcatgacatcagcctctgccctccctaatttgtggcccctgtgggacaagttcactgtccagctggccctggcccagctcagcaggcatgcagctgagtgctcttccctccttctgtcaccttcaggccagtcttcctctctctgacttgggctgagcctggatgggaggctgagaaaatccagtttccatgccaagaggcagcaggtcacagcccatgcttcgagattagaaggggtccctctcagtgtccagcctcatgtagctgggctaggagctaggttggatctgtgtgactccttcctctggaggtatgggccaggagcggctgagccctgggagcagccatatgcagtatgtgtttgtatccagaaggaggagggacatgggaggaggggctgtgatgaggggtgctcagaggagcctgctcctgagaagcagagccacgcacccacctcgggggtttctgagctggggtagtggcaggaggggaattccagcctcaaacttcctgtctcagctgctactcggttcctgcccggccaccccccctccccaggggcaggcgggctagcccccccatcatatgcaaatcatggggaaattcacctctgtttccttctaagggaatttttttccactcgccaggcccagcccaggcatatcttttcacctggggaggctggcccagccctgccttctgtcctggggcaggagcagcctagctggagtcttctacacacacatcctatcagtgtctgcttagcccggctgggggaccctgaccttacaggcaaagagaactggaatgggggcagggatgtctggttttcctgtgccagcagcgcttcctccagtgcagtgaaagagactaaagctagctgtaggggggcactccaggcctcatgtgctctaccaggaggagggctctgatagcgcccactgatcagtttttccatctgctgagggaatattcaggtgctagggctgctgagaaccgacttgtcccagccaagattttcagtgcccacaggcagtgcactgccctgagagccctggcatcccattagttcccagtgctccaagatactagcctaggatcatgtgcccggagggaggcaggtgggcggatacatgcgcacactgcatacctgagacaaggagccaggcctgggatgacccagctgccatggttttgagaggcaggccagaaggcccaaggaaggggggtgatgtgatgggggaagcactgggcattgtgaagacacagaccagggacctgacctattgtggcaggtcagggaaggcttctcagaggcagtgacatctcacaagggttctgaaggaagagcaggagtttgccagcagcattgtttctcccgttcgccacctgtctctcataacacactttgttcaaactcaattttgggagctttgttctcccaaataaattctggatcttctgttagagacaggaaggcaaccgagtggcccagacctgggttcgagttctgactgtctcttaccactagccaggtgaccttcctttccttcacggtagcaagccctataagaatgaagccatatatctggccaccagcgggagctcagtgcctgcgaatccctgctcattccctgcgatcagggtctgtgcctgccttcgtacctgcctgagcattgacgtacagggtagtgataagcataggccctgggcttgcgtctctcttccaccgttaactgctgtgtgaccctggtgactaagctgccttctctggaccacagttttcatctgaaaaacagggaaggagttgatgatggtatagtgccctcctcacaagcttgctgatgattcagtgttagacacttataacagggcctggttcacagctctcagcaaatgggaacttttattataagcaggctggcagatgtggcagagggaccgggaaggatgagtgctgccctgggctggactttgtctcccctacaatttagagaccttcagaccctggtgccccagtcttgcccccaaatagcctccatggcccccagctgccccacctccctccctagcttctcttagggctccagctatcaccccttacctcccccactcccattaaagacacccacaggatgtggttggccctggacttgcctcttttgcgtaacatttattctgttttatttgccaaatacgagagtctaagctgttcactgagggggcagtgagacgtggggctgctggtcctaggagctcttccatgacacctagtgttacctgggagccccaagccccagtcccccatccctcaggaaatccagttggaacccctactttgcagggccatctttggcctgaagccaccaggaaagctgtcccactgactgacccctcttaaggaagctgcagactgctgggcccattttcagatagaggaacggagagctgcggcagcccaggagccctgccaggcttccttctgggtgcagccacctgtgggtggggcgcggcctcgtctgagaccaaggcagggcccatgccttactcagctgtccttgctaagagtcccctagcatcttcctgatggtctttctgccttggggtgaagaggcatttgggggaattgccatcttgggaggaaaagcagtatcctttcactttcagactgaaaggctcctggggaacttgcttcactccggcgctttctgtcccatgctgaaggctggagtcgccagccccttcccctttgcctttctcaggccccagtccctgaggtgtgtgtgaggactgaactttaatccggaacaatctggtttctcctcggtcagcccagccggtgcttcccccgcccccacccacgctgcaagaggcgagggaggggtggtgactcagtgtgcatgtgtacatctgtccctgtggtcccggcagctcgccgggtggggcaggaaccagaagtctcgcggcacctttcccccacccccaggtgtgttgcaagtggcctggccctcccccaccgtggggagcagtgctgccagcttcctgggcgcttcctcactttcaggagatggagcctcagggaaagtcccagcggggcctggccatcctggagctgtgggaactggcactgggaatggaggccttggccttgggccccacctgcagagggccagggcctgagccctggggaaaggggaaggggctggtgactccaaaggggaagggactgctcactaccaccaccaaagccagggcaggggtggggtgtgaccacctccctcctcctgctcaggcctagggggtggcaggctggccatcagtgtgggctaaccctgtcctctccctcccagccatgggaacaaggaggtcttctcctgccggggcatcctgctggcagtgaactggtttctggagcggggccacacagacatcacagtgtttgtgccatcctggaggaaggagcagcctcggcccgacgtgcccatcacaggtgagtggtgcctctggaggtgggatggtcttactgccccagcagccctggttctccccaagagagaccctctgggagtgacctacaacttctagaactcaagcagggaccagcatttcttttctcagttttttctgtccttagcaacctgctctgaatggtggtcattggaggtcaccttgtccagtcttctgcctcagatctggagcccctccctcatcctactggtgggcaggtcgctctttcaacatccccagtggcagaagcgcagtatcacatagtggttaagcctgtgggctctgagtcgagacagccagagttggaatcctggctctgtcccctcttggcgccctggcctggacagggacccctcttagccctgctctgagccacatgctcctcatctgaaacatggggtcggtgtcagcagggggtgttgacagcattgccttcctcgtgaggtcatataagttaacacacggtaagtgctgagaagagtacctggcacatggtagatgctcagtgtatgttagctgttaccactgttcttgcaagagcccccagcctttggaggtaaaaactgaatcagtttctctagaagtcttgagatctgggggcagcctgtctgggactgtaggctccaggaagaggggccctttccacttgcacacttctgtgtccctgtggtcccagcagctccccaggtggggcatggtgagtggcctgaccttttcccaccatgggatttagcgaccttgtcctggcaatgcaagggacagagccaagatacttctcccaccccagagcaagctgcggctcattcacggacccctcagaacaggctggatgaaagccaaagccaagaccacatctggcttgaacaggcctgatgtccagaaggagaaaaggaacaaggccagctctgtgcttggagcctctgccatgcaggccctgccgcctgtgttcagatgttcttggtcttcaggcagcaaactgtgccacgccaagggaggggctggggcatggaagaggcctcagcagagttagttcttgccccggtgaccttggcgttaaccactcctgtgtgtggccatcagcaggttagggagccctgtaggcctggctctaggtcccgctgggccctgacctgtgtgcacccgtcacctcccagaccagcacatcctgcgggaactggagaagaagaagatcctggtgttcacaccatcacgacgcgtgggtggcaagcgggtggtgtgctatgacgacagattcattgtgaagctggcctacgagtctgacgggatcgtggtttccaacgacacataccgtgacctccaaggcgagcggcaggagtggaagcgcttcatcgaggagcggctgctcatgtactccttcgtcaatgacaagtatgttccctcccagaggccctgacagacttggggtccacaggggaagccagaggtgcccttggcaagggtggagctgggggctgggctctgcggggccctgtggccatgggaggttgcgggtcttggctccaggcagctttgagagtgagacggatagctcaccacataggagaaatcagaccgggaccaggcaggctgtggggtggagagagtggctaatttgggagatagagccgtagcacttatgaggggatgtatgtggttgatggttccaggtggcctctctacgaaccaacatggcatctctcgagcagaggccatgggccagtgggtgcgggctgccatcccccgacgacctcagagagggagttcccctaaaggtgcccatgggctgtggccctctagaccggggatccctttctcctcccttggtatgatcccacccatagagcagttttgcggggtcctggacaggccccagttttaggtgtcactctcctattcttcccagcaaggcttgccatcggccccttctcagcaattagaagtccctgcatggctcccacctccagagagttctttgcaccctctgcaggtttatgccccctgatgacccactgggccggcacgggcccagcctggacaacttcctgcgtaagaagccactcactttggagcacaggaagcagccgtgtccctatggtatggaacccagcctgccctccccggccctcctcactcctgcccttcctctcacctgtctgcctgtgccctgttttcctcttggggacctccaccattggaccacccaaccccgtttcctgtgccaccccttccctgctctcatccctggttctgaagtgcccctgcttagagtccctcttgattcctcttccaggaaggaaatgcacctatgggatcaagtgccgattcttccacccagagcggccaagctgcccccagcgctctgtggcagatgagctccgtgccaatgctctcctctcaccccccagagccccaagcaaggacaaaaatggccggcggccttcaccttcatcccagtccagctctctgctaacagagagtgagcagtgcagcctggatgggaagaagctgggggcccaggcatccccagggtcccgccaagagggtctaacacagacctatgccccatcaggcaggagcctcgcacctagcgggggcagtggcagcagctttgggcccacagactggctcccacagacgctggactcactcccgtacgtctcccaggattgcctggactcgggcattggctccctggagagccagatgtcggaactttggggggttcgaggaggaggccctggtgagccgggcccaccccgagccccttacacgggctacagtccctatggatctgagctcccagccaccgcagccttctctgcctttggccgggccatgggtgctggccacttcagtgtccctgccgactacccacccgcgccccctgcctttccacctcgagagtactggtctgaaccatacccactgcccccacccacatcagtccttcaggagcccccagtgcagagcccaggggctggcaggagcccgtggggcagggcaggcagcctggccaaggagcaggccagcgtgtatactaagctgtgtggtgtgtttcccccgcacctggtggaggctgtgatggggcgcttcccacagctcctggacccccagcagctggctgccgagatcctctcctacaagtcccagcaccccagtgagtaagctgcctgtggctggcaagggcagcacccccagcctccaagggccgtcaggctgggctttgggccattgagcagcccattcccagccctgaggcccaccccagaggctggacagagggaggattcaagtcgggaaggaaacccacaaaccaaagatactgtaggattggttctggcccatgcagcacctctagctgtctgcctcagtgggtcagaagcgatcaccctgttgatacacattgtatctctgtagtttaaggagacgctgccggtaacggcgtcggtccgtggctgaggcccaaaccgtcttttctctcagagggtggggagggaggtgggggcagcagaggcctgggctgggtgccctgtgcacgccaccccacttccgccctacccctgggacgttggccttggctggctagttgggcaccgtgtgcctgccctccaagggcctcctctacgccaatgaggcctcatctgtgctctcgctgggcacgtggcttcatgtcagtaagcaagatgcttcttaataacccaccttctgccccactctattccttatcctgctgcccctgtaggggtcaagggccctccgtctacaccctcttcttctcctccatcctttattcagagtcatctcgcccttccccatgggtgggggaacctgtgtttgtttgtgtgcacatgtaaattttaaatattttaagcagaaagtccttacctcctgtaacacatcaataaagtacaatcattgtgagccctttcaa

SEQ ID NO: 6 - DNA, 9468 bp, Mus musculus
ttccgcgtcattgcccttaaagcacctgcccgcctgtcgcggctacgaaggtgccgtcagagcccgagactcggctccatggacaccttacagacgacgtccggcgctcagcggggtgagtgctaccgggtggaacgggtgctgcgctgcgttgggtctgactccgggagggtcgcgcgcgggcagtgtctccatcgctgggaccccgctgagttcaccctggtgcgtggaacggggtgtcttggggcacccggcgttctccgggctctagccaggacctcctcgcgtcaccgggcccggtagccaaccagagtcgcccagcaacttttctctccgtgggtcaccccggaaacttgtccttcccagaatagaactctgtccctgagccgaagtatgagactcggagacgaaggcggtggccacaatggggaagcccctgggtcccctgcgcacttccagccccagtttccttctgagagccccgcccctgcctcttgggtcccttgggttggcatggaacagaacggggtttgggggtggagtcagaccaagttcaaatgtttgtatcacaaactttgagttgatgatcttgagtaaacgcttactgcctgtgcctcggtctccctcttgtaaaaataactaatgctgagagctctttgagagcacagcgtgggctgacacgtgagaatggctttgaatagcgtcttctcagttcgtgttgccccattctgtggaagagagtctgaaagagacctatattgggcattgcttgcccttaaccctgtggtctggtcaataggagctgtgatggacacattccaagaatgtgaaatatgagtgacccttgtggaacgaagcctgtccaagaatccaaccccaccatgagtctgtggagtcttgaggacagacacagcagccagggtcgacctcagccagaccaggatcctgtggctaaagaggcccctacttccgagctgcagatgaaggtggactttttccgtaaactggggtactcgtcctctgagatccacagtgtcctgcagaagctgggagtccaagcagacaccaacacggtgctaggggaattggtgaagcatggctcagctactgaacgagaatgccaggccctgacggcccccagcccccagccccctctggtgccccggggtggaagcacccccaagccttccactctagaaccctcactcccagaggaggacagagagggcagcgacctgagacctgtggtcatcgacggaagcaatgtggccatgaggtatgtgtggagacctgtggaatagtgtctgtagctcttcctttgggaagggggcttggcttttctcttggactggtgaaggaacacaaaagctgtctgtgagatcccaccatgtgccagaaggaaccccttaagccttaaagacttcctcagggctgtttctccaatgctataagaaggaaaacagctcaaatccttgccaagagtcacacttcttgataaatgagaagcaaagattcaaacccagccttctttgcctggagtgtaccttgtgccatggtctatccttgcctacggcatgaggcaggacagtataagggattaacccgtgggccagtcacttagccagttgggctttttcttctcctgtcaaactttccactcaggtggcccctgtggggagaaaattcatagaacgtggagagttacaagtactcagtggagccgggcgtggtggcacacacctttaatcccagcacttgggaggcagaacaggcagatttctgagttcaaggccagcctggtctacagagtgagttccaggatagccaggttctatacagagaaaccctgtctcaaaaaagaagaagtactcagtggacagatgttattccatgacttgagtcttctgggatctccttgtccccgactcgagctcccactgaagcaaggctcattagttattcagagcttcctagccctaaccagctgtgaccttttggtcagatctggcatcttcagatgcgtgtgtacgggtagcctgatgctctgggctcaggtgtgctgtaatgagaatctatactccctgaagccgctcaggctggcctgacctctgggggttcccagatctcctgccgcctttgcatatctctcaaggaatggggtggggtggaggcagaatgggagccaggctgcctctgaccttaaggagccctgatttctcccttctatccaggccgcaccctccagctctgctttctcccagctccctgcacagcgggctggtgagtcaggactgggtggataaagtacaaggaaaagatcctgttggcagggaccaggggagaccccagcaaaagaacaggaagaggagaagtctcctttctagatcccctgagggctctcccaagaggactgcccaacttgttatgatgtcagcttcttcgccacctggctcctggtcctacctactcccagcccagctgggtcaagtgagctgtccagctggtcctggccctacttagctggttcatggaggcagcccttctctaccacctttgggtctgttgtcacatctttaaaatgggccgatcctgggctggaaggctgagaaatgctggcttctctgccagagggcagaagggtcaactggttaccttccgagtctcgagttctctctgaggtcctgaattaggaggggggtctgacttcccctgagagtcctaggctgggtgttaggagtcttgtgggcagccatgtgctctgggtatttgtttctgggcaggaggaggaggggcctgggtgggaggaggggcagtagtggaggggttcaagagagctgataccaaagagacagacagccactgtctgccctcttgcctgcctgcctatctgctgggggtttctgagctagggtggccacggaaggggaattccagcctcacacttcctgtctcagcagctgcttggttcctaccctgctgctacacacacacacacacacacacacacacacagacacacacaagggcaggccggctaacccccaccatatgcaaatcgtggggaaattcacctctgtttccttctaagggaattttcctccacaggagttaggcccagcccaggttgttctctccacctggggaggctagccctgccttgtccttgggcaggaatgggagctttccagagtcacggccctgtcagggtggggggccctgaactgtggaccagcaggactctgtgggtgggaggaagttggttttcctaggccaaccaggcttcctgcattgcagtgagctgaatgagctgtccaggcggtctgggcctcaggcctcaagggctctccctgccaagaatagggctctgactacctgctgagggaactgtcccagtgccagcactgctgagggcagacgtgtccccaacctgggttttaaatgcccacaggcggtatacagcctaagcaccctggcgtcttgagatcctccaacttgtctttgctggtccagggagggctcgtgagtccacatgtggacactgcacgctcaggacagcgagccatggggcagatgcttacctcacacctgagcgaggcagtgtgatgggacagcagtgggcattgtgaagccccagactgggccctgccctggcaggtgcggaggtggggtggggtggagtggggaggggttctcggaggttttgacatatcacttggcttccagaaagaagagaagaattggcagcaaatggctcatcatttctctgtctgtggggcggggaggcatctccctctccatatgcagaacacacaccatcattgataattgagttcagaaactgctcttctcccacatgaactttgggtcttctgtaagggacagccagcgctccaaccagtagccccactccctggttcagttttcaccacttggcagtagccagggaacttgtccaagtgacggaagtcttcctttctagacagagtgatggtcaaggtggactccgtgcctttcccacctatcagggctgttggtacctaagcaacaacagggtagttctgatcactggctactgtgtgaccctagtgactactaagttaccctctctggaccacaggagcggaggcagtgtacttctggttttgcttgctgatgattccgtgtagagcgcggaatggccaggtttgcagctccctgtgaatgttaaccgacttatgagcaagccgcctgggaccaggagggcgcgagggctcccttggggtgggctttcttcctctccaactcagagacctctagctagaccttggagccttgccctgagatagtgtccattcccactctgttctcttggggctcaagctatcgctcccccaaagccacccacaggatgtggtctgccatggacttgcctcttttgcgtaacatgtattctgttttatttgccaaatatgagtctgagtcgctcactctccccagagtaacaagaggcagggctttctggggagggagccccaagctccagtcctccttcagcactcccagaaaatccaccttgccagcgcctctggcctcaagccttagggagctctgtgtccctggggtgaaagagcttgtctgcaaaagctgtccaccaagcttgtgggcagagacacagaccagggcaagcagctaggctgcttccgaggaaagccgtgtttagatagaacaaggccagggcctgatgccccagccctcctgtgagcagagttccccagtaatttcctgcccatcatctgccaggggtcacgccctccctggggcggggggtaggggagggcagggaggggcagggcagtctttgcagggaacaggcctcttaagaggaaaagaggtggcctttcactttcacacggaaaagctcctggtgctgttatttcctgaccccttgtggaaggacgctccccgtgcccctgagacctcatgcagactgaactttaatcctcaacagtctggtttctcctgcttaacatggccggtgcctccctcccccgcctcccttgctggtgcctccttgctgtgtagtgttgagggaaagaggtgactcagcctgcatgtgcacatccacgtccccgtggtccctgcagcttgggtggggcaggatccccaagtctcccatcacttttccccacccccaggtatgttgcaaatggcccgaccctccccctctacgtggggagccagtgtggctagattcctgggtggatccacgcttttaggagatggaatcttagggaaagtcccttccaggggctggccatcaggcactgtgggaaccggcccagggacaagaccttaggccctactggcagagggccatgggctgagtcctacagttgcccagcaaggccaagcagggctagattgtggctcccttcttacttttgctctgaccggactgactacatacagactggctactctgtgagctaacctttccctcctctgccagccatgggaacaaggaagtcttctcttgccggggcattctgctggctgtgaactggtttctggagcgaggccacacagatattaccgtgtttgtgccatcttggaggaaggaacagcctcgaccagatgtgcctatcacaggtatgtatgccctctggaagtaggatagcctcactctactagggaccctggtgcttccccaaagggtccttaaatagggtcagcaccttctaaactgtaatgaggaccagcctgtctttttcaattcttgttgctttcagcagcccagttcaggcaggaaatggctggaagtcaccttgtgtcgctttgggatgccttcctgatcctattggagtggggtgtcccttcttcaacatccccggtggcagagggacagtgtagcttcatatttgaaattgtgggtgctgggccaagaagacagggtaaagggcttggccctaagttactcaggtaagggcttcaagtagacattagctgtgggctcttccatctgtgaaatgggacagggtagaggtggcatcctccaaacactgctgaaggagcagttgagaatggtacagaagtgcttaactcagtgcagtgtgaaagctcggcatagtgtgaatgcccagctgcgaccattgctctcatgaaagcccccaccttgaggaggagggagtgacaagaggcagctttttaagaagtctgcataggctggagagatggctcagtggttaagaacactgactgcccttccagaggtcctgagttcaaatcccaggaactacacggtggctgacaaccatctgtaatgggatctgatgccctcttctggtgtgtctgagctacagagtactctaaataaattattaagaaaaaaaaataaggttggcttagataaaaaccatagactacattttaaaaaaaaaataaagaagtctgcaaatcagggccatgctaggggcttctaggccttttctgaacagagctgggagtgttgttttgagtgtgtagaggacagggccaagacacttcttcctggacccctgtggtacaggcaaggcacacaggtcggaggggaagtaggaagagccactcttggctccgcactcttgctgaatgtgatggccctgcatagagatgcacttggttttcacacagctggtttccgtcctcttggaactggtgtgaacagcctctcatcccggccactgtgagccatgggaccctgtgggtccatcttagggtctccaatgggccctgacctgcagctctgctcatcccccagaccagcacatccttcgggaactagagaaaaagaagatcttggtgttcacgccatccaggcgggtcggcggcaagcgcgtggtgtgctatgatgaccgcttcattgtgaagctggccttcgaatccgacggagtggtggtctccaatgacacgtaccgagacctccaaggcgagaggcaggagtggaaacgcttcatcgaggagcggctgctcatgtactccttcgtcaatgacaagtacgttactgctggggactggctctgctgggcccacaggctgggctgcagctttcggggtgacatgagttgcagggagaaggctagggacagtggaactttttgatagggagcctgggagttgaagctggcccgagacagaaccaattcgttcctaggtggctctctcaagaaggtctatggccacacacatcccaagatacacttagccttcctgatagcctcatagttcccagcctagggagtgccccgaaggcgaccctggcctataccctctggactgagggatcccctcctcctctggggtgacccaactgcaaactcagaagactttaggggcatcctgcagacaagcatcttgggagctggctatctaaccctttccttggttcctcttcacgacttctgtttcctgagcgttctgcttcacctaccacaggttcatgccccctgacgaccctttaggacggcatgggcctagcctggacaacttccttcgtaagaaaccactgccttctgagcacaggaagcagccatgcccctatggtgagacccagctctgtcctcaatagtcttacttaccctacctttcctcttgcctgtcccatcccacttaggcccgcccgtggcttccttgtggggaccccctgttctagaccacctgaatcccaagtctgtgcctcgtcctcgctctgaccccatttagacaccctctctgttcttgcagggaagaaatgtacgtatggaatcaagtgccgatttttccaccctgagcggccaagccgtccccagcgctctgtggccgatgagctccgtgccaacgctctcctctcacctcccaggactccagtcaaggacaaaagtagccagaggccttcccctgcctctcagtccagctctgtgtccctagaggctgaaccaggcagcctggatgggaaaaagctgggtgccaggtcatctccgggtccccaccgagaaggctcaccgcagacctgtgctccagctggcaggagcctccctgttagtgggggcagctttgggcccacagagtggcttgcacacacccaggactcactcccatacacctcccaggagtgccttgattcaggcattggttccctggagagccagatgtcagaattatggggcgtgcgaggaggcagccctggggagtcgggccccactcggggcccctatgcaggttatcacagctatggatccaaggtcccagcagcaccttccttttctccttttagaccagccatgggtgctggccacttcagtgtccccaccgactatgtgcccccgccacccacctacccatccagagagtactggtctgagccgtacccattacccccacccactcctgtccttcaggagccccagagacccagccccggggctggtgggggcccctggggcagggtgggtgacctggccaaagaaagggctggtgtatataccaagctgtgtggtgtcttccccccacacctggtagaagctgtaatgagacgcttcccacagctgctggatccgcagcagctggccgcagagatcctgtcttacaagtcccagcacctcagtgagtaagccagaaggtggcgcaaggggctgtcgggcatcacagatagcggtccccagcccctgcctggcctaccctggaagctggacagaaggacaagtgaagcaggcaagggaatcctcaaaccaaagataccataggattggttctggccccgcggcaccccaacccgtcccgcacaggtcagaagtgatcaccctgttgatacacattgtatctctgtcatttaaggagacgctgccggtcaggccgtccatccgtggctgatgcccaaaccctcttttttttttctcagagggccgggtgggaggcatgggggagcagaggcctgagctggaccccaccttccaccctgtccctgggacgccggcaccaccggctagttaggcaccatgtgcctgctctctgaggccccctcaagccaatgcggcctcatccctgttcacagggcatgaggcttcatgttagtaagcaagatgcttctttaagcccctcccctgcccgctctgtccacctacacaccccccccccaaccagggctccaaggccctctgtttccacacctcccatgggtgggaggacacatgtatgctgtgtacagaggcgagatttaaatattttaaatgaaaaaggttgacaaaataaaggctattgccaggcaggctggagagatggctcagtggttaagagcaccgactgctcttctgaaggtcctgagttcaaatgtcagcaaccacatggtggctcacaaacatctgtgatgagatctggtgccctcttctggggtgtctgaagacagctacagtgtacttacatacaataataaatcttttaaaaaagagagaaatttaaaagaaaaaaaaagctattgcc

SEQ ID NO: 7 - DNA, 2949 bp, Homo sapiens
atggcaaactcaatgaatggcagaaaccctggtggtcgaggaggaaatccccgaaaaggtcgaattttgggtattattgatgctattcaggatgcagttggaccccctaagcaagctgccgcagatcgcaggaccgtggagaagacttggaagctcatggacaaagtggtaagactgtgccaaaatcccaaacttcagttgaaaaatagcccaccatatatacttgatattttgcctgatacatatcagcatttacgacttatattgagtaaatatgatgacaaccagaaacttgcccaactcagtgagaatgagtactttaaaatctacattgatagccttatgaaaaagtcaaaacgggcaataagactctttaaagaaggcaaggagagaatgtatgaagaacagtcacaggacagacgaaatctcacaaaactgtcccttatcttcagtcacatgctggcagaaatcaaagcaatctttcccaatggtcaattccagggagataactttcgtatcacaaaagcagatgctgctgaattctggagaaagttttttggagacaaaactatcgtaccatggaaagtattcagacagtgccttcatgaggtccaccagattagctctagcctggaagcaatggctctaaaatcaacaattgatttaacttgcaatgattacatttcagtttttgaatttgatatttttaccaggctgtttcagccttggggctctattttgcggaattggaatttcttagctgtgacacatccaggttacatggcatttctcacatatgatgaagttaaagcacgactacagaaatatagcaccaaacccggaagctatattttccggttaagttgcactcgattgggacagtgggccattggctatgtgactggggatgggaatatcttacagaccatacctcataacaagcccttatttcaagccctgattgatggcagcagggaaggattttatctttatcctgatgggaggagttataatcctgatttaactggattatgtgaacctacacctcatgaccatataaaagttacacaggaacaatatgaattatattgtgaaatgggctccacttttcagctctgtaagatttgtgcagagaatgacaaagatgtcaagattgagccttgtgggcatttgatgtgcacctcttgccttacggcatggcaggagtcggatggtcagggctgccctttctgtcgttgtgaaataaaaggaactgagcccataatcgtggacccctttgatccaagagatgaaggctccaggtgttgcagcatcattgacccctttggcatgccgatgctagacttggacgacgatgatgatcgtgaggagtccttgatgatgaatcggttggcaaacgtccgaaagtgcactgacaggcagaactcaccagtcacatcaccaggatcctctccccttgcccagagaagaaagccacagcctgacccactccagatcccacatctaagcctgccacccgtgcctcctcgcctggatctaattcagaaaggcatagttagatctccctgtggcagcccaacaggttcaccaaagtcttctccttgcatggtgagaaaacaagataaaccactcccagcaccacctcctcccttaagagatcctcctccaccgccacctgaaagacctccaccaatcccaccagacaatagactgagtagacacatccatcatgtggaaagcgtgccttccagagacccgccaatgcctcttgaagcatggtgccctcgggatgtgtttgggactaatcagcttgtgggatgtcgactcctaggggagggctctccaaaacctggaatcacagcgagttcaaatgtcaatggaaggcacagtagagtgggctctgacccagtgcttatgcggaaacacagacgccatgatttgcctttagaaggagctaaggtcttttccaatggtcaccttggaagtgaagaatatgatgttcctccccggctttctcctcctcctccagttaccaccctcctccctagcataaagtgtactggtccgttagcaaattctctttcagagaaaacaagagacccagtagaggaagatgatgatgaatacaagattccttcatcccaccctgtttccctgaattcacaaccatctcattgtcataatgtaaaacctcctgttcggtcctgtgataatggtcactgtatgctgaatggaacacatggtccatcttcagagaagaaatcaaacatccctgacttaagcatatatttaaagggagatgtttttgattcagcctctgatcccgtgccattaccacctgccaggcctccaactcgggacaatccaaagcatggttcttcactcaacaggacgccctctgattatgatcttctcatccctccattaggtgaagatgcttttgatgccctccctccatctctcccacctcccccacctcctgcaaggcatagtctcattgaacattcaaaacctcctggctccagtagccggccatcctcaggacaggatctttttcttcttccttcagatccctttgttgatctagcaagtggccaagttcctttgcctcctgctagaaggttaccaggtgaaaatgtcaaaactaacagaacatcacaggactatgatcagcttccttcatgttcagatggttcacaggcaccagccagaccccctaaaccacgaccgcgcaggactgcaccagaaattcaccacagaaaaccccatgggcctgaggcggcattggaaaatgtcgatgcaaaaattgcaaaactcatgggagagggttatgcctttgaagaggtgaagagagccttagagatagcccagaataatgtcgaagttgcccggagcatcctccgagaatttgccttccctcctccagtatccccacgtctaaatctatag

SEQ ID NO: 8 - DNA, 2949 bp, Mus musculus
atggcaaattctatgaatggcagaaatcctggtggtcgaggaggaaacccccgcaaaggtcgcattttggggattattgatgctattcaggatgcagtcggacccccaaagcaagctgcagctgaccgcaggactgtggagaagacttggaaactcatggacaaagtggtaagactgtgccaaaatcccaagcttcagttgaaaaacagcccaccgtatatacttgatattttacctgatacgtatcagcacttgagacttatattgagtaaatatgatgacaaccagaagctggctcaactgagcgagaatgagtactttaaaatctacatcgatagtctcatgaagaagtcgaagcgagcgatccggctctttaaagaaggcaaggaaaggatgtacgaagagcagtcgcaggacagacggaatctcacaaagctgtcccttatcttcagtcacatgctggcagaaatcaaggcgatctttcccaatggccagttccagggagataacttccggatcaccaaagcagatgctgctgagttctggaggaagttttttggagacaaaactattgtaccatggaaagtcttcagacagtgcctgcatgaggtccatcagatcagctctggcctggaagcaatggctctgaagtcaaccattgatttaacttgcaatgattacatctcagtgtttgaatttgatatttttaccaggctatttcagccctggggctctattttacggaattggaacttcttggctgtgactcacccgggatacatggcatttctcacgtatgatgaagttaaagctcggctacagaaatacagcaccaagcctggaagttacattttccggttaagctgcactcggctgggacaatgggccattggctatgtgactggggacggcaatatcctacagaccatacctcataacaagcccctgttccaagccctgattgatggtagcagggaaggcttttatctttatcctgatgggcggagctataaccctgatttaaccggattatgtgaacctacacctcacgatcatataaaagttacacaggagcagtatgaactgtattgtgaaatgggctccacttttcagctctgcaagatctgtgcagagaatgacaaagatgtcaagattgagccttgcgggcatctgatgtgcacttcgtgccttaccgcgtggcaggagtctgatggccaaggctgccccttctgtcgctgtgagataaaaggaacggagcccatcattgtggaccccttcgaccccagagatgaaggctccaggtgctgcagcatcattgaccctttcagcatccccatgcttgacttggacgatgacgatgatcgagaggagtctttgatgatgaacaggctggcgagtgttcgaaagtgcacggacaggcagaactcaccagtcacatcgccaggctcctcaccccttgcccagagaagaaagcctcagccagaccctctccagatcccccacctcagcctgccaccagtgcctccccgcctagatctcattcagaaaggcatcgtgcgctctccgtgtggcagccccacaggctcgccaaagtcttctccatgcatggttagaaaacaagacaaaccactcccggcaccccctcctcccttgagagatccacctcctccaccagagaggcctccccccatcccacctgacaatagactgagcagacacttccaccatggagagagtgtgccttcccgggaccagcccatgcccctcgaagcctggtgccctcgggatgccttcgggactaaccaggtgatgggatgtcgcatcctcggggatggctctccaaagcctggcgtcaccgcaaactccagcttaaatgggaggcacagtagaatgggctccgagcaggttcttatgaggaaacacagacgccatgatttgccttcagaaggagccaaggtcttttccaatggccaccttgccactgaagaatacgatgtccctcctcggctttcccctccgcctccagtcaccacccttctcccaagcataaagtgtactggtccattagcaaattgtctctcagagaaaactagagacacggtagaagatgatgacgatgaatacaagattccttcatcccatcctgtgtccctgaattcacaaccatctcattgccataatgtcaaagctcctgttcggtcttgtgataatggtcattgtatattgaatggaactcatggtgcgccttcagagatgaagaaatcgaacatcccagatttaggcatctatttgaagggaggtggttctgactcagcctctgatccagtaccgttaccacctgccaggcctccaccccgggacagcccaaagcacggctcttcagtcaacaggacgccctctgactatgatctgctcatccctccattaggtgaggacgcctttgatgctctccctccatccctccctcctcccccacctcctgcaagacacagtctcatcgaacattcaaaacctccaggctccagtagccggccttcctcagggcaggaccttttccttcttccttcagatcccttttttgacccaacaagtggccaagttccattgcctcctgccaggagagcagcaggagacagcggcaaagccaacagagcctcgcaggactatgaccagctcccttcatcttccgatggttcgcaagcaccagctagaccccccaaaccacgaccccgaaggactgcaccagaaattcatcacagaaagccccatgggcccgaagcggcgttggaaaatgtcgatgcaaaaattgcaaaactcatgggagaggggtatgcctttgaagaggtgaagagagccttagagattgcccagaataacgtggaagtggccaggagcatacttcgagaatttgccttcccccctcctgtctccccacgtctgaatctatag

SEQ ID NO: 9 - DNA, 11102 bp, Homo sapiens
ctttttttcaaagattttgattccagtggaatctttgctttagatttgtgtgtgtgtttgtgttcgtgaattgtaactccagaagcaatgcctgtacaagctccacaatggacggatttcctctcctgcccaatttgcactcagactttcgacgaaacaattcgaaagcccatcagtttgggttgtggccatactgtctgcaagatgtgcctgaataaactccaccgcaaggcttgcccatttgaccagaccactatcaatacagacattgagctcctccctgtgaactcagcattgctgcagctcgtgggtgctcaggtccctgagcagcagcctattactttgtgtagtggggttgaagacacaaagcattatgaggaagccaagaaatgtgtagaagaattagcattgtacttaaaaccgctcagcagtgctagaggagtgggtctgaacagcactactcagagtgttctgagtcgcccaatgcagaggaagcttgtgactctagtccactgtcaactagtagaagaagaaggcaggattcgtgccatgagggcagctcgatctttaggtgaacgaacagttacagagctcattctccagcaccagaatcctcagcaactctcttccaatctttgggcagcagtaagggctaggggatgccagttccttggaccagcaatgcaggaggaagctttaaagttggttctgctggccttagaagatggttctgctttgtcaagaaaagtattggttctgtttgtggtgcaaagattggagccacggtttcctcaagcctctaaaactagcattgggcatgttgttcagctcctttatagagcctcctgtttcaaggtcaccaaacgtgatgaagactcttctttgatgcagctgaaagaagaatttagaacctatgaagctctgcggcgagaacatgactcccagatagtgcagattgctatggaagcaggcttacgaattgcaccggaccagtggtcctctttgctttatggagaccagtctcacaaatctcatatgcagtccattattgacaagttgcagactccagcctcttttgcacagagtgttcaggaactaacaattgctctccagcgaactggagacccagcaaacttgaaccgactaagaccccatttggagctgttagcaaacattgaccctagtccagatgctcctcctcctacttgggaacagctggaaaatgggctggttgctgtgcgtacagtggtacatgggctggttgattatatccagaaccacagcaaaaaaggagcagatcagcagcagcctccacagcatagcaaatacaaaacatacatgtgtcgagatatgaagcagagaggaggatgccctcgtggggccagctgtacatttgcacactcacaggaggaactggaaaaatttcgtaaaatgaataagcgcctggttccgagaagacccttgagtgcctctttgggtcaacttaatgaggtgggcctgccttcagcagctatccttccagatgaaggtgcagtggatctccctagcagaaaacctcctgctctgccaaatggaattgtatcaacagggaatacagtaacacagcttattccgcgagggacagaccccagctatgattctagtctgaaaccaggaaaaatagatcatctgagcagtagtgctcctggatcccctcctgacctgctagaatctgttcctaagagtatttctgccttacctgtgaatccacattctatacctccaagggggccagcagatctgcctccaatgcctgttaccaaaccacttcagatggtacctcgaggttctcagttatatccagcacaacagacggatgtttattatcaggatcctcgaggagcagctccgccatttgaaccagcaccttatcagcagggtatgtattatactccaccaccacaatgtgtgtcccgctttgtccgacctccaccatctgctcctgaacctgctcctccctacttggatcattatccaccctacctccaagaacgtgttgtaaactctcagtatggcacacagccacagcagtacccacctatatacccatctcactatgatggccgtcgagtgtaccctgctccgtcttacacaagagaagagatattccgagaaagccctatacccattgagattccacctgcagcagtaccatcgtatgtaccagaatccagagaaagataccaacagatcgagagttactatccagtggctcctcatccaactcagatcagaccttcgtacctcagagaacctccttatagccggcttcctcctcctccccagcctcatcctagtctagatgaactacatcgccgacgaaaggaaataatggcccagctagaggaaagaaaggttatctctccacctccttttgcaccttcaccaaccttgcctcctacctttcatccggaagaatttttggatgaagacttgaaggtagctgggaaatacaaaggaaatgattatagccaatactctccctggtcatgtgacaccatcggctcctacattggaaccaaagatgcaaaacccaaagatgttgtggcagcagggagtgtggaaatgatgaatgtggagagtaaaggaatgagggaccagcgattagatcttcagagaagagcagcagaaaccagtgatgatgacctcatcccatttggagaccgaccaacagtgtctcggtttggtgccatctcacgaacttccaaaactatatatcagggtgctggtccaatgcaggctatggcacctcagggagctcctacaaaatctattaacatttcagattatagtccatatggaacccacggtggctggggagcttctccatattcacctcatcaaaacataccttctcagggacacttcagtgagagggagagaatatctatgtcagaagtggccagtcatggaaaaccccttccatctgctgagagagaacagctacgactagaattgcagcaattgaaccatcagattagccagcagacccagctacgtggactagagagggaggcaaacaccctggcaggccagtcacagcccccaccaccgccacctccaaaatggcctgggatgatctcaagtgagcagttgagcttggaactgcaccaggtggaaagggaaatcgggaagagaacacgggaactgagtatggaaaaccagtgttctctggacatgaaaagcaaactgaatacaagtaaacaagcagaaaatggacaaccagaaccacaaaacaaggttccggctgaggaccttacattgacattcagcagtgatgtaccaaatggatcagccttgacacaagagaatatcagcctcctatcaaacaagaccagctctctgaacctgtcagaggaccctgagggaggaggggataataatgactcccagagatcaggagttactcccagttctgctccttaaaatatgaggagtcctacttttatcttctgctcctaatcagtttgtcgggcataggggtaggagaacgcataacttctaaactttttctcttaagagcggtcagagaaatccaggaaattcaccagaggcaatgtgcacaaacaccactactaaaaacaaaccctgcacatagttcacattaatgcatttttttaatttaaagaaaaagaaaattagcagtatctgcaagcaattcagattaaatttgtttttgttctgtgaatgaactttattttaataactttggacgccttggatccagggctttaaaaaaaaaagatattatatatacactctgtttgattaattgtatgatcttaatgaagtaggttttcttgtattttggtattacatacccagtgtataatttgtataattcctcacccccaatccttatcaattctcaccaccccataaacttaaaacagaccaaaaatgtcagctttcagagaattctgtggtgcttgagaaacagacagtactagactgagttattagaaaatgtgcagcaaaccaacttttcccagagctgttatttggaattctttatccctttctagatcactagcttcattggatacgtatatttttcttgacattgaagctttttgtttgtttgggagggttttatttcttgtttttattttcaacttttcaaatagaattaccaattttgaagtcctcattttcatattaaggatcatttgcattctcttaatataatcagaaagctgattcagtgtttaattagatttaaatgttgatcaagaattaactgtaacttactagcactgaaaatacatgtttggtgtttcgcctgcgttcagctcatggttggaaatttgaagcagtgcttttcaaattaaacttttgctgacatcataggatgctaaaaaacaaggacaatcaactatgttttaaaactgcaacatttgatttgaacttaaaatgaatagagccacattattttaatggatgttttgttttacttgtgaactgattgagtcttaaggatagtttatgaattgatcacttgttgctcagaatcattagtataagtaggtaggaaatagtttgaagtcatttttcatagacttaactgtccaatgttgcaaatgtttcttcatgaaaatattgaaaagagtaaggttttttttttttttaattctgctgcacatattctgatgccatgtgtctgtgcttaattaactaagaccctataaaacgtgtttagtggggactgtttgatatagttgccctctccattcctctatttgcctgatttttttcagtttccctttctgtattttactactaaattagaaagcactggttatgtaataagttactacattgctttggttgagtaaaaacaagagtttatatttttcttgtttcttattttcttaacccttaactcctgctgaggtcataacaacctcagtctaagctctgtgtccatcatactgttaacttaaacaaatgggtggtgggggtgggaagggtaatacagtcctgccattgcctaccagtaggagagtccaaacagaacactcttttgagtagcaattatttaatttgcccagtcaaggcaccgtgtttatatactatttcacattgaatttgattatgccctacagacctggctggtcaaggatttgatatacacatattggcttgggattcgagctttcttttttatttaaataaaaatttatatatatattatatatatacatatatacatagctatatctgtatatatattgggtatgttttaaggattttttcgcatgagcgcagctgttgcgataaattgaccgattgggaggtggtagaagcactgaatgaaggtgaggctttttttttttccagttttcttatacatttgtcttctttttttaaaaaaaaaaaaaaaaatatatatatatatatatatatatatatatatgtatgctttttcttttttttttggcttttgtttgctttttgacttttacttaaaacccttttacatttccactttaaatgcctattgtttattgggagaaggaaagaaacttttgtatgtctgacagagtcccaagatttttcatttctctgcataacagtaaaaggcaacattattggcctccaaccttctttttttttttttttttttttttttggagagggagtctgcttctgttgcccaggctggagtgcagtgggttgatctcggctcgctacaacctccacctcccgggttcaagcgattctcctgcctcagcctcctgagtagctgggactacaggcacgcgccaccacgtccagctaatttttgtactacaaccttcttataagcagatttggactgatcttctcctttgggtatgtcagatcatcctaaattttagaagcagttgattcactttggaattcagaatgattcttgtattagaagataatttgtgggtattaatttttgtttaagtttaaaataaaagtaaagattcattttggatatcagttgaaaccccttagtaactcagtttctgttattcttgttctcatttcctttaaatacacttgttcttggcttttgccattttgattctgtgaagtaggcaggagcagggattaatttatacagtattcctgttctgaacaaaaccagaaaagtcactgtataaacttgacttaaaatagtatctttctcttttcatgtattttcatttgggggaaaaaaaatctctttaattgtaacctgaattcaagctgtacccctccatggtcctacactctagagctaatctggttgggcagaaaggcagaaggatggtatattgtcccattgtgcctataatgtattttaaattggtcattccaccttacctaatggaaattcttgcagctttcctagtgctcatcagcggttttaggaattcactaacacccccaaaatttgttttttgttttttgctttttttccttttccttttctctgtgatatcatgagcctgtaaggacaaatggcatatttgggcataactgcttgactctgtaactacttttttaaaatcaaaattgaaaaggtattaattgcttctggctttttaatttttttttttagagacaaagatgaaaccagataggaaatacagtttttatgttttctacttactaaaatattagtttcttctaaatattactttatttgtatttcactaaattccaaagcaaaaatcagatatctgagatgaacaattttgaatgggttaaggtaggagtaataagcattgtgttcactcatacataggtgagggaattttatttatagcgagaaatataaaagtattttgacttactcttttttcaaggagtcgctaggaaagggtagaagaactgagaataagacatctagattccatttctggtttatatgtagctcttttgcctaatttatatataattttgtcataagataagtccagtaactgtcctgtcttagataacgtacagaggctgaagtgaccaaagatccattccttattacttttctaattaaaagaagagttagctagatttctagtagtagtattctagatattgataagacattattcctaatgccaactacaaatattcaactactcagaatgtatttaaaaaattgtttggtacagcattaatgttggtgattcctttctcccatcttatttcccttcccttctattctttcccatgtatttcttttgcctaaaagaatattggtatgattctcatttgctactttgagtatataactccatcagactttccagagttcttcagggatggagctcaaagtttacatttttccatatatgtctttttttttcttgacctttttccaccctgtaatttccccttgacttacccactgccctccccaatctgcaacagtgacagtttgctgcctcaaaacagagcattcaaatgaattcgcttagccaataacttctgtgaagttgccttttctaatggtgttattactcagtgatgcacaaatgtggtatttgccctactggtaggcaaacaaaggtctggttaagaatggtaacctgctgttttctttaaaggagagccaaagacttgtgctttactctgttttggttttgggggaagtagtagccattttgatcatgaaaccttttagtatttgtgagttgattgtactgtgtaagtagatttatctaaagttaactgttaaagttagctagccttataaaatgtaaaagtaatgaaaattcatcagtttctctaagccagtgaatgtcgtcttttgtaatattgtgtataagcatttgtgcccaccccagcttctgctatcactgtggtcaagtcatgggtctgagtaaaagaattcttgagtatgtgactggtgagaaataactaactatagtcagtacatgtacagattgttaaaattcaagacaaaatactttcactaaaaaaaaggattcgtctcagtaaagaagtattcagttgctgtcacttacatacaaatggtggtgggtagcagtctcaccatttttctactctctttttgctcgttttaaataattgagtaaaggttttatttgtaaaatctatctgtaaaaagcacctgtaacgatttatatgtcattgaaaggaatggcccagttactttttaggagagattaataacctacctggaaattaactgcagtgtttacttagattttttccccctaataagagtctaagtgtgttaaatttttaactgatgatatggttacagtgttgcaaggtgatctcttcccctgaaaaatctgcctatccttttaagaaaaattagacctactaaactaaagcaaattttactttcatcattagcacctaaacaagcaagcagtctactactgtcatcattttacctgagtgcccattttgctgatccacattcatacctttctaccttggacagctatgatttttatatttatttgttgcttcagctcaggtttatgaaattttcctgctgaaatgacttattgggagttgtaggttgattatagttactattaaacattttctcaatttagcttttctgagccacttcaagaagaaagctgtttttgtcaaaaggtactattaattgtagcaagtaaatcttaagatttgaatttttaagattttatttctgtgttcactaaagatctttccacaagtaatagattataattgataagtaatactagctatagtgttaataaagagtaagactatcaccaccaggaataagtaacaccttttaataataaatctattcctaataggtatgttttcaatactgcactgaacaaagtgggtaaagagccctttgaaatcttattgtaataaagggacttacctcaccactggaatccttgttttcagagcctctggattttcagccaaacactgaatttctcctaaactttatccctgaaacatccctagagaacgaactttattttcacaagaaaataaggcacaataagggggaagaacttaaacctcaacataatatcgtgtccaaaaatgtgtatttagaaaaataaaatcctgtggagtttgttatcctaatgtttattttatactagataaggtgacttgttttcttgtattgaggtcttactgtttcagatttgtttttgtttctgttctaactctaagcagacagagctcacaagtctttggcatccattttaggttgtacactcagagtcatcttataactggaaactgtcaggtggtcccccaccccagtgagtgtatgaaagagtcaagaaaaagattggaaacaaactattgggtgaataattggttgctctattattattattattattatttgcctttttttaatcaccatttatttgtgtaatctttctccattgattggctgtcattcttataatatctgaactaccccataaaataattctctgacttaaaggcatttagcttttgcttatcgtttttacatcctctattcaactaagacactgtcttgagagttattttttccagatggatcgttggcctaaattttcacacttctcccctgttcatcctttttcctcttccctgcttcctgggaataaaaggaacttttttaaaaaaattaattagtccacaggtctcattatctttctttatgattaatctatgactttttggtacaagaacaatggaaaaagtgaattaaggtaatgaacaaaacctttcacccacttaaacattttccagttttgagattcctcttcgtgtttgtggtgtcttccccttgttaccccttctgccctttttctctgactatggtaatttggtctttaggctcatatcagtctccccgagacattctgcagtcattatcacctttttgggtggattttattttgttttattttgttttttttaaaaaaataactttttaacattggtgcatatttgcttgggatagagcttgtgtaatttaccaatcgtattgattgtaagtgattgtgccctgcagaggtatatttaacaagacaaaaataatcttggttaataaaggagcccatgagatttgagtcaggttgtaagtgaaatcacttacacttttggatagaatttatactcctgctcttataaatcagtggtagacttaccattttttaaagttttcttgcatttttttgtttttttattgccacagctccctattctttcttgcctgcctccacccccctgttcaggaaaaaaaaaaattgagccttaaagtgacagctgattttttaattgctgaattttgtgaaattttactttttccaagtgtttccaactttaaaaagagaagtgaagacaaataggttggaatggtgaagacaaatggattggaatttcacaggctgtgaataattccttaggatctggcaaaccgtgaagtcttatttgaagaccttatctcctgagagttcttttggagtaggaaaaagaaccctatttgaaatagaccgtttttctcttgtttttaatctgtttaatatttctgatttttaagcagctttcaaaacaagtgtggtggaaaaaaagaaatagtagtaggaagatgtttagggcagcagaactctgggtctaaataagtacatgttcccacttgttgccgatttttgagagtactagggccatctttctcaattttgtattatttgtgtgcatgtttatatcaaagatgcccattttgttaaaatgctatttcctttattaccttggaaactgactcagcctcatgttgctcctaattagtgtttaaggctcccatgagttgcagataaaatgatttattttaacaagtagaaggaggtgattcaccttttggattgtaaatatatgaaaatgtctacaaggtctttatctgctttctgtcagcatttatattaaatgataaattaatgaggaacgtgtgtattttgtaaatgagtgtgtgccatcttattctaatcttgagattactttcttacacgtttttaatggagggggcttttttaggtgaaaaataaagaaatggctaatctgcatgagcaaacaca

SEQ ID NO: 10 - DNA, 3610 bp, Mus musculus
gctcggaccagagcggttatatttcacactatgtgctgactgtatctacagaagatatttaaaaaaaagataaacttttttccaagattttgattccagtggaatctttgctttagattttttgtgtgtgtgtgcgtgttagtgaattgtaactccagaagcaatgcctgtacaagctccacaatggacggatttcctttcctgcccaatttgcactcagactttcgacgaaacaattcgaaagcccatcagtttgggctgtggccatactgtctgcaaaatgtgcctgaataaactccaccgtaaggcttgcccatttgaccagaccactatcaatacggacattgagcttcttcctgtgaactcagcattactgcagctagtgggtgctcagatccctgagcaacagcccattactttgtgtagtggggttgaagatacaaagcattatgaggaagccaagaaatgtgtagaagaattagcattgtacctgaagccactcagcagtgcgagaggtgtgggtctaaatagcactactcagagtgttctgagtcgcccaatgcagaggaaacttgtgactctggttcattgccagctagtggaagaagaaggcaggatccgtgctatgagggctgctcgatctttaggtgaacgcacagttacagagctcattctccagcaccagaatcctcagcaactctcttctaatctctgggcagcagtcagagctaggggctgccagttccttggaccagcgatgcaggaggaagctctgaagctggtcttgctggccttagaagatggttctgcattgtctcggaaagtgttggttctcttcgtggtgcaaagactggagccacgttttcctcaagcctctaaaaccagcattgggcatgttgttcagctcctctacagagcctcctgcttcaaggtcaccaaacgtgatgaagactcttctttgatgcaactgaaagaagaatttaggacttacgaagctctgaggagagaacatgactcccagatagtgcaaattgctatggaagctggcttaaggattgcaccagaccagtggtcctctttgctttatggggatcagtctcacaaatctcatatgcagtctattattgacaagttacagacccctgcctcatttgcacagagtgttcaggagctaacaattgctctccagcggactggagacccagcgaatttgaaccgactaagaccccatttggagttactcgcaaacattgaccctagtccagatgctcctcctccaacatgggaacagctagaaaatgggctggttgctgtacgtacggtagtgcatggattggttgattatatccagaaccacagcaaaaaaggagcagatcaacaacagcctccacagcatagcaaatacaaaacatacatgtgtcgagatatgaagcaaaggggaggatgccctcgtggtgccagctgtacgtttgcacattcacaggaagaactggaaaagtttcgtaaaatgaacaaacgtctggttcccagaagacccttgagtgcctctttgggtcaacttaatgaggtgggcctgccttcagcacctatactttctgatgaaagtgcagtggacttgtccaacagaaaacctcctgcactcccaaatggaattgcatcgtcagggagcacagtgacacaactgattccacgtgggacagaccccagctttgattcttctctgaaaccagtaaagctagaccacctgagcagtagtgctccggggtcccctcctgacctgctggaatctgcccctaagagtatttctgccttacctgtgaatccacatcctgtacctccaaggggaccaacagatctgcctcccatgcctgtcaccaaaccaattcagatggtacctcgaggttctcagctatatccagcacaacaagcagatgtttattatcaggatcctcgaggaagtgccccagcttttgagacagcaccttaccagcagggtatgtactatactccaccaccatgtgtgtcccgctttgtccgacctccaccatctgctcctgaacctggtcctccatacttggatcattattcaccctacctccaagatcgtgtcataaactctcagtatggcacacagccacaacagtacccacctatgtacccagctcactacgatggccgccgtgtataccctgctcagtcttacacaagagaggagatgttccgggaaagccctatacccattgacattccatctgcagcagtgccgtcctatgtacctgagtccagagaacggtaccaacaggtagagggttactacccggtggctcctcatccagctcagatcagaccttcataccccagggatcctccttatagtcggcttcctcctccccaaccccatcctagtctggatgagctacatcgaaggcggaaggaaataatggcccagctagaggaaagaaaagtcatctctccacctccttttgcaccgtcaccaacattgcctcctgcattccatccagaggaatttttggatgaagacttgaaggtagccggaaaatacaaagcaaatgattatagccagtattctccttggtcatgtgataccatcggctcctacattggaaccaaagatgcaaaacccaaagatgttgtggcagcggggagtgtggaaatgatgaatgtagaaagtaaaggaacgagagagcagaggttggaccttcagagaagagcagtggagacgagcgatgatgacctcatcccgtttggagaccgaccaacagtctctcgattcggtgccatctctcgaacatccaaaacactgtatcagggtgctggcccactgcaagctatagcacctcagggagcacccacaaaatctattaacatttccgattacagtgcatatggagctcatggtggctggggagattctccatactcacctcatgcaaacatacctcctcagggacacttcattgagagggagaaaatgtccatggcagaagtggccagtcatggaaaaccccttctatctgctgaaagagaacaattacggctagaattgcagcagctgaaccatcagatcagccagcagacccagctacgaggactagaggctgttagtaaccggctggtgttacagagggaggtgaacaccctggcaagccagccacagccccctcagctgcctccaaaatggcctggaatgatctcaagtgagcagctcagcttggaactgcatcaggtggaaagagaaattgggaagaggacccgggaattgagtatggagaaccagtgttctgtagacatgaaaagcaaactgggtacaagcaagcaagcagaaaatggccagcccgagccccaaaacaagatccgcactgaggaccttacattgacattcagtgatgtaccaaatggatcagccttgacacaagagaatctcagcctcctatcaaacaagaccagctctttgaatctgtcagaagactctgagggaggaggagataacaatgattcccagagatcaggagttgtttccaattctgctccctagaatatgggcacctgcttctaccttctgctcctaatcagttcatggggcataggg

SEQ ID NO: 11 - DNA, 1559 bp, Homo sapiens
acagcagtccgtgccgccgtcccgcccgccagcgccccagcgaggaagcagcgcgcagcccgcggcccagcgcacccgcagcagcgcccgcagctcgtccgcgccatgttccaggcggccgagcgcccccaggagtgggccatggagggcccccgcgacgggctgaagaaggagcggctactggacgaccgccacgacagcggcctggactccatgaaagacgaggagtacgagcagatggtcaaggagctgcaggagatccgcctcgagccgcaggaggtgccgcgcggctcggagccctggaagcagcagctcaccgaggacggggactcgttcctgcacttggccatcatccatgaagaaaaggcactgaccatggaagtgatccgccaggtgaagggagacctggccttcctcaacttccagaacaacctgcagcagactccactccacttggctgtgatcaccaaccagccagaaattgctgaggcacttctgggagctggctgtgatcctgagctccgagactttcgaggaaatacccccctacaccttgcctgtgagcagggctgcctggccagcgtgggagtcctgactcagtcctgcaccaccccgcacctccactccatcctgaaggctaccaactacaatggccacacgtgtctacacttagcctctatccatggctacctgggcatcgtggagcttttggtgtccttgggtgctgatgtcaatgctcaggagccctgtaatggccggactgcccttcacctcgcagtggacctgcaaaatcctgacctggtgtcactcctgttgaagtgtggggctgatgtcaacagagttacctaccagggctattctccctaccagctcacctggggccgcccaagcacccggatacagcagcagctgggccagctgacactagaaaaccttcagatgctgccagagagtgaggatgaggagagctatgacacagagtcagagttcacggagttcacagaggacgagctgccctatgatgactgtgtgtttggaggccagcgtctgacgttatgagcgcaaaggggctgaaagaacatggacttgtatatttgtacaaaaaaaaagttttatttttctaaaaaaagaaaaaagaagaaaaaatttaaagggtgtacttatatccacactgcacactgcctggcccaaaacgtcttattgtggtaggatcagccctcattttgttgcttttgtgaactttttgtaggggacgagaaagatcattgaaattctgagaaaacttcttttaaacctcacctttgtggggtttttggagaaggttatcaaaaatttcatggaaggaccacattttatatttattgtgcttcgagtgactgaccccagtggtatcctgtgacatgtaacagccaggagtgttaagcgttcagtgatgtggggtgaaaagttactacctgtcaaggtttgtgttaccctcctgtaaatggtgtacataatgtattgttggtaattattttggtacttttatgatgtatatttattaaacagatttttacaaa

SEQ ID NO: 12 - DNA, 1083 bp, Mus musculus
cgccagccagccgccagcagcctgcagcctgcacccgctcagccccgcacagccatgtttcagccagctgggcacggccaggactgggccatggagggcccgcgggatggcctcaagaaggagcgcttggtggacgatcgccacgacagcggcctggactccatgaaggacgaggagtacgagcaaatggtgaaggagctgcgggagatccgcctgcagccgcaggaggcgccgctggccgccgagccctggaagcagcagctcacggaggacggagactcgttcctgcacttggcaatcatccacgaagagaagccgctgaccatggaagtcattggtcaggtgaagggagacctggccttcctcaacttccagaacaacctgcagcagactccactccacttggctgtgatcaccaaccagccaggaattgctgaggcacttctgaaagctggctgtgatcctgagctccgagactttcgaggaaatacccctctacatcttgcctgtgagcagggctgcctggccagtgtagcagtcttgacgcagacctgcacaccccagcatctccactccgtcctgcaggccaccaactacaatggccacacgtgtctgcacctagcctctatccacggctacctggccatcgtggagcacttggtgactttgggtgctgatgtcaacgctcaggagccctgcaatggccggacagccctccaccttgcggtggacctgcagaatcctgacctggtttcgctcttgttgaaatgtggggctgatgtcaacagggtaacctaccaaggctactccccctaccagcttacctggggccgcccaagtacccggatacagcagcagctgggccagctgaccctggaaaatctccagatgctacccgagagcgaggatgaggagagctatgacacggagtcagaattcacagaggatgagctgccctatgatgactgtgtgtttggaggccagcgtctgacattataagtggaaagtggcaaaaagaatgtggacttgtatatttgtacaaatagagttttatttttctaaaaaaaaaaaaaaaaaaaaaaa

SEQ ID NO: 13 - RNA, 23 nt, Homo sapiens
cgcacuuccgcacauuccguucg

SEQ ID NO: 14 - RNA, 23 nt, Homo sapiens
ggggagggucucuggcuuuauuu

SEQ ID NO: 15 - RNA, 23 nt, Homo sapiens
cagcauuaacugggaugccgugu

SEQ ID NO: 16 - RNA, 23 nt, Homo sapiens
ccaggaccugaacucgcaccucc

SEQ ID NO: 17 - RNA, 23 nt, Homo sapiens
uacauauacccaguaucuuugca

SEQ ID NO: 18 - RNA, 23 nt, Homo sapiens
gccgacaaugcagucuccacagc

SEQ ID NO: 19 - RNA, 23 nt, Homo sapiens
ccccugguuguuguagcagcuua

SEQ ID NO: 20 - RNA, 23 nt, Homo sapiens
cugcugugcagaauccuauuuua

SEQ ID NO: 21 - RNA, 23 nt, Homo sapiens
ugggaugccguguuauuuuguua

SEQ ID NO: 22 - RNA, 23 nt, Homo sapiens
ucgcaccuccuaccucuucaugu

SEQ ID NO: 23 - DNA, 19 bp, Homo sapiens
cacgcacttccgcacattc

SEQ ID NO: 24 - DNA, 19 bp, Homo sapiens
ttccgttcgcacgccgatt

SEQ ID NO: 25 - DNA, 19 bp, Homo sapiens
gagcttcgactgcctcttc

SEQ ID NO: 26 - DNA, 23 bp, Homo sapiens
cgcacttccgcacattccgttcg

SEQ ID NO: 27 - DNA, 23 bp, Homo sapiens
ggggagggtctctggctttattt

SEQ ID NO: 28 - DNA, 23 bp, Homo sapiens
cagcattaactgggatgccgtgt

SEQ ID NO: 29 - DNA, 23 bp, Homo sapiens
ccaggacctgaactcgcacctcc

SEQ ID NO: 30 - DNA, 23 bp, Homo sapiens
tacatatacccagtatctttgca

SEQ ID NO: 31 - DNA, 23 bp, Homo sapiens
gccgacaatgcagtctccacagc

SEQ ID NO: 32 - DNA, 23 bp, Homo sapiens
cccctggttgttgtagcagctta

SEQ ID NO: 33 - DNA, 23 bp, Homo sapiens
ctgctgtgcagaatcctatttta

SEQ ID NO: 34 - DNA, 23 bp, Homo sapiens
tgggatgccgtgttattttgtta

SEQ ID NO: 35 - DNA, 23 bp, Homo sapiens
tcgcacctcctacctcttcatgt

SEQ ID NO: 36 - DNA, 21 bp, Homo sapiens
tttcgagctgctggagcacta

SEQ ID NO: 37 - DNA, 21 bp, Homo sapiens
tcgagctgctggagcactacg

SEQ ID NO: 38 - DNA, 21 bp, Homo sapiens
tcgccaacggaactgcttctt

SEQ ID NO: 39 - DNA, 21 bp, Homo sapiens
acttctggctggagacctcat

SEQ ID NO: 40 - DNA, 21 bp, Homo sapiens
gcgagaccttcgactgccttt

SEQ ID NO: 41 - DNA, 21 bp, Homo sapiens
cgacactcacttccgcacctt

SEQ ID NO: 42 - DNA, 21 bp, Homo sapiens
ctacctgagttccttcccctt

SEQ ID NO: 43 - DNA, 21 bp, Homo sapiens
ttccgctcccactccgattac

SEQ ID NO: 44 - DNA, 21 bp, Homo sapiens
taacccggtactccgtgacta

SEQ ID NO: 45 - DNA, 21 bp, Homo sapiens
tactccgtgactacctgagtt

SEQ ID NO: 46 - DNA, 21 bp, Homo sapiens
cttccgctcccactccgatta

SEQ ID NO: 47 - DNA, 21 bp, Homo sapiens
gcgcgacagtcgccaacggaa

SEQ ID NO: 48 - DNA, 21 bp, Homo sapiens
tggacgcctgcggcttctatt

SEQ ID NO: 49 - DNA, 21 bp, Homo sapiens
cgcatccctcttaacccggta

SEQ ID NO: 50 - DNA, 21 bp, Homo sapiens
tacatattcccagtatctttg

SEQ ID NO: 51 - DNA, 21 bp, Homo sapiens
gcgccttattatttcttatta

SEQ ID NO: 52 - DNA, 21 bp, Homo sapiens
ccgtgactacctgagttcctt

SEQ ID NO: 53 - DNA, 21 bp, Homo sapiens
ggagggtctctggcttcattt

SEQ ID NO: 54 - DNA, 21 bp, Homo sapiens
ttcgcgctcagcgtgaagatg

SEQ ID NO: 55 - DNA, 21 bp, Homo sapiens
atccctcttaacccggtactc

SEQ ID NO: 56 - DNA, 20 bp, Homo sapiens
gcggctgcgcgccgagcccg

SEQ ID NO: 57 - DNA, 20 bp, Homo sapiens
ggacgcctgcggattctact

SEQ ID NO: 58 - DNA, 20 bp, Homo sapiens
ggctgccatccaggtgaaag

SEQ ID NO: 59 - DNA, 20 bp, Homo sapiens
gcggctgtcgcgcaccagga

SEQ ID NO: 60 - DNA, 20 bp, Homo sapiens
tggacgcctgcggattctac

SEQ ID NO: 61 - DNA, 20 bp, Homo sapiens
gacgcctgcggattctactg

SEQ ID NO: 62 - DNA, 20 bp, Homo sapiens
agtgctccagcagctcgaag

SEQ ID NO: 63 - DNA, 20 bp, Homo sapiens
gccggccgctttcacctgga

SEQ ID NO: 64 - DNA, 20 bp, Homo sapiens
agtagaatccgcaggcgtcc

SEQ ID NO: 65 - DNA, 20 bp, Homo sapiens
cgcaccaggaaggtgcccac

SEQ ID NO: 66 - DNA, 20 bp, Homo sapiens
ggccggcctgaaagtgcacg

SEQ ID NO: 67 - DNA, 20 bp, Homo sapiens
tccgttcgcacgccgattac

SEQ ID NO: 68 - DNA, 20 bp, Homo sapiens
agcgcgctcctggacgcctg

SEQ ID NO: 69 - DNA, 20 bp, Homo sapiens
cggctgcgcgccgagcccgt

SEQ ID NO: 70 - DNA, 20 bp, Homo sapiens
acgcctgcggattctactgg

SEQ ID NO: 71 - DNA, 20 bp, Homo sapiens
cgaggccatcttcacgctaa

SEQ ID NO: 72 - DNA, 20 bp, Homo sapiens
tcaggccggccgctttcacc

SEQ ID NO: 73 - DNA, 20 bp, Homo sapiens
cttagcgtgaagatggcctc

SEQ ID NO: 74 - DNA, 20 bp, Homo sapiens
gccggtaatcggcgtgcgaa

SEQ ID NO: 75 - DNA, 20 bp, Homo sapiens
ctgcattgtcggctgccacc

SEQ ID NO: 76 - DNA, 20 bp, Homo sapiens
gtgcgccccgtgcacgctca

SEQ ID NO: 77 - DNA, 20 bp, Homo sapiens
gctgtgccgccagcgcatcg

SEQ ID NO: 78 - DNA, 20 bp, Homo sapiens
cacgcggcgctggcgcagcg

SEQ ID NO: 79 - DNA, 20 bp, Homo sapiens
gctcctgcagcggccgcacg

SEQ ID NO: 80 - DNA, 20 bp, Homo sapiens
agctctcgcggctgccatcc

SEQ ID NO: 81 - DNA, 20 bp, Homo sapiens
tggtgcgcgacagccgccag

SEQ ID NO: 82 - DNA, 20 bp, Homo sapiens
gatggtagcacacaaccagg

SEQ ID NO: 83 - DNA, 20 bp, Homo sapiens
agaggcagtcgaagctctcg

SEQ ID NO: 84 - DNA, 20 bp, Homo sapiens
gctggcggctgtcgcgcacc

SEQ ID NO: 85 - DNA, 20 bp, Homo sapiens
ccgaggccatcttcacgcta

SEQ ID NO: 86 - DNA, 20 bp, Homo sapiens
ggggcccccagcatgcggcg

SEQ ID NO: 87 - DNA, 20 bp, Homo sapiens
gctgctggagcactacgtgg

SEQ ID NO: 88 - DNA, 20 bp, Homo sapiens
cgagctgctggagcactacg

SEQ ID NO: 89 - DNA, 20 bp, Homo sapiens
cgaaaaagcagttccgctgg

SEQ ID NO: 90 - DNA, 20 bp, Homo sapiens
gcaggcgtccaggagcgcgc

SEQ ID NO: 91 - DNA, 20 bp, Homo sapiens
ggggcccctgagcgtgcacg

SEQ ID NO: 92 - DNA, 20 bp, Homo sapiens
gcggcgccgcgccgcatgct

SEQ ID NO: 93 - DNA, 20 bp, Homo sapiens
gcacgcggcgctggcgcagc

SEQ ID NO: 94 - DNA, 20 bp, Homo sapiens
tgggggcccctgagcgtgca

SEQ ID NO: 95 - DNA, 20 bp, Homo sapiens
caggaaggtgcccacgggct

SEQ ID NO: 96 - DNA, 20 bp, Homo sapiens
tgcgccccgtgcacgctcag

SEQ ID NO: 97 - DNA, 20 bp, Homo sapiens
gccatccaggtgaaagcggc

SEQ ID NO: 98 - DNA, 20 bp, Homo sapiens
cacgcgcgccagcgcgctcc

SEQ ID NO: 99 - DNA, 20 bp, Homo sapiens
gggcccccagtagaatccgc

SEQ ID NO: 100 - DNA, 20 bp, Homo sapiens
atccgcgtgcactttcaggc

SEQ ID NO: 101 - DNA, 20 bp, Homo sapiens
cgagcccgtgggcaccttcc

SEQ ID NO: 102 - DNA, 20 bp, Homo sapiens
ccacagcagcagagccccga

SEQ ID NO: 103 - DNA, 20 bp, Homo sapiens
agccaggttctcgcggccca

SEQ ID NO: 104 - DNA, 20 bp, Homo sapiens
aaagtgcacgcggatgctcg

SEQ ID NO: 105 - DNA, 20 bp, Homo sapiens
ctcttcctcctcctcgcccg

SEQ ID NO: 106 - DNA, 20 bp, Homo sapiens
gcgtgcacggggcgcacgag

SEQ ID NO: 107 - DNA, 20 bp, Homo sapiens
aagtgcacgcggatgctcgt

SEQ ID NO: 108 - DNA, 20 bp, Homo sapiens
cgtgcgccccgtgcacgctc

SEQ ID NO: 109 - DNA, 20 bp, Homo sapiens
gcagcggccgcacgcggcgc

SEQ ID NO: 110 - DNA, 20 bp, Homo sapiens
ccttagcgtgaagatggcct

SEQ ID NO: 111 - DNA, 20 bp, Homo sapiens
caggttctcgcggcccacgg

SEQ ID NO: 112 - DNA, 20 bp, Homo sapiens
gcgcaccaggaaggtgccca

SEQ ID NO: 113 - DNA, 20 bp, Homo sapiens
gctgccggtcaaatctggaa

SEQ ID NO: 114 - DNA, 20 bp, Homo sapiens
cggcgtgcgaacggaatgtg

SEQ ID NO: 115 - DNA, 20 bp, Homo sapiens
cagcagcagagccccgacgg

SEQ ID NO: 116 - DNA, 20 bp, Homo sapiens
gggcgaaaaagcagttccgc

SEQ ID NO: 117 - DNA, 20 bp, Homo sapiens
cgcacgcggcgctggcgcag

SEQ ID NO: 118 - DNA, 20 bp, Homo sapiens
ggatgcgagccaggttctcg

SEQ ID NO: 119 - DNA, 20 bp, Homo sapiens
tggcggcacagctcctgcag

SEQ ID NO: 120 - DNA, 20 bp, Homo sapiens
gcgcccgcggccgtgccccg

SEQ ID NO: 121 - DNA, 20 bp, Homo sapiens
ggcgccgcgccgcatgctgg

SEQ ID NO: 122 - DNA, 20 bp, Homo sapiens
cggtggccacgatgcgctgg

SEQ ID NO: 123 - DNA, 20 bp, Homo sapiens
tgctgtggagactgcattgt

SEQ ID NO: 124 - DNA, 20 bp, Homo sapiens
taggatggtagcacacaacc

SEQ ID NO: 125 - DNA, 20 bp, Homo sapiens
gcggccgtgccccgcggtcc

SEQ ID NO: 126 - DNA, 20 bp, Homo sapiens
gagcatccgcgtgcactttc

SEQ ID NO: 127 - DNA, 20 bp, Homo sapiens
cgctgccggtcaaatctgga

SEQ ID NO: 128 - DNA, 20 bp, Homo sapiens
cagcgcatcgtggccaccgt

SEQ ID NO: 129 - DNA, 20 bp, Homo sapiens
gcggatgctcgtgggtcccg

SEQ ID NO: 130 - DNA, 20 bp, Homo sapiens
cggcgccgcgccgcatgctg

SEQ ID NO: 131 - DNA, 20 bp, Homo sapiens
cggtcaaatctggaagggga

SEQ ID NO: 132 - DNA, 20 bp, Homo sapiens
aggaaggttctggccgccgt

SEQ ID NO: 133 - DNA, 20 bp, Homo sapiens
ccacggtggccacgatgcgc

SEQ ID NO: 134 - DNA, 20 bp, Homo sapiens
cgctgcgccagcgccgcgtg

SEQ ID NO: 135 - DNA, 20 bp, Homo sapiens
aggagctcaggtagtcgcgg

SEQ ID NO: 136 - DNA, 20 bp, Homo sapiens
gcagcggggcccccagcatg

SEQ ID NO: 137 - DNA, 20 bp, Homo sapiens
ggaaggagctcaggtagtcg

SEQ ID NO: 138 - DNA, 20 bp, Homo sapiens
tcgcggaggacggggttgag

SEQ ID NO: 139 - DNA, 20 bp, Homo sapiens
cgactgcctcttcgagctgc

SEQ ID NO: 140 - DNA, 20 bp, Homo sapiens
gcgccgcgtgcggccgctgc

SEQ ID NO: 141 - DNA, 20 bp, Homo sapiens
caccgtgggccgcgagaacc

SEQ ID NO: 142 - DNA, 20 bp, Homo sapiens
gtgccccgcggtcccggccc

SEQ ID NO: 143 - DNA, 20 bp, Homo sapiens
ctgccggtcaaatctggaag

SEQ ID NO: 144 - DNA, 20 bp, Homo sapiens
cttccccttccagatttgac

SEQ ID NO: 145 - DNA, 20 bp, Homo sapiens
ctcaggtagtcgcggaggac

SEQ ID NO: 146 - DNA, 20 bp, Homo sapiens
cgggcgctgccggtcaaatc

SEQ ID NO: 147 - DNA, 20 bp, Homo sapiens
ggaaggttctggccgccgtc

SEQ ID NO: 148 - DNA, 20 bp, Homo sapiens
gctcaggtagtcgcggagga

SEQ ID NO: 149 - DNA, 20 bp, Homo sapiens
gcggaagtgcgtgtcgccgg

SEQ ID NO: 150 - DNA, 20 bp, Homo sapiens
ggaccgcggggcacggccgc

SEQ ID NO: 151 - DNA, 20 bp, Homo sapiens
gggaccgcggggcacggccg

SEQ ID NO: 152 - DNA, 20 bp, Homo sapiens
gcgcgtgatgcgccggtaat

SEQ ID NO: 153 - DNA, 20 bp, Homo sapiens
tcaggtagtcgcggaggacg

SEQ ID NO: 154 - DNA, 20 bp, Homo sapiens
tgcggaagtgcgtgtcgccg

SEQ ID NO: 155 - DNA, 20 bp, Homo sapiens
ggggccgggaccgcggggca

SEQ ID NO: 156 - DNA, 20 bp, Homo sapiens
ccgtcggggctctgctgctg

SEQ ID NO: 157 - DNA, 20 bp, Homo sapiens
gaaggttctggccgccgtcg

SEQ ID NO: 158 - DNA, 20 bp, Homo sapiens
gtgtgctaccatcctacaga

SEQ ID NO: 159 - DNA, 20 bp, Homo sapiens
gtcgcggaggacggggttga

SEQ ID NO: 160 - DNA, 20 bp, Homo sapiens
cgctggcgcgcgtgatgcgc

SEQ ID NO: 161 - DNA, 20 bp, Homo sapiens
gcgtgcacggcgggcgctgc

SEQ ID NO: 162 - DNA, 20 bp, Homo sapiens
tctggaaggggaaggagctc

SEQ ID NO: 163 - DNA, 20 bp, Homo sapiens
gtgcgtgtcgccgggggccg

SEQ ID NO: 164 - DNA, 20 bp, Homo sapiens
gggcacggccgcgggcgcgc

SEQ ID NO: 165 - DNA, 20 bp, Homo sapiens
gttaatgctgcgtgcacggc

SEQ ID NO: 166 - DNA, 20 bp, Homo sapiens
gcacggccgcgggcgcgcgg

SEQ ID NO: 167 - DNA, 20 bp, Homo sapiens
ggggcacggccgcgggcgcg

SEQ ID NO: 168 - DNA, 20 bp, Homo sapiens
gtgcggaagtgcgtgtcgcc

SEQ ID NO: 169 - DNA, 20 bp, Homo sapiens
gaggaagaggaggaaggttc

SEQ ID NO: 170 - DNA, 20 bp, Homo sapiens
ggctggccccttctgtagga

SEQ ID NO: 171 - DNA, 20 bp, Homo sapiens
ggggccggggccgggaccgc

SEQ ID NO: 172 - DNA, 20 bp, Homo sapiens
cgcggaggacggggttgagg

SEQ ID NO: 173 - DNA, 20 bp, Homo sapiens
tttcgcccttagcgtgaaga

SEQ ID NO: 174 - DNA, 20 bp, Homo sapiens
ggcacggccgcgggcgcgcg

SEQ ID NO: 175 - DNA, 20 bp, Homo sapiens
agtcgcggaggacggggttg

SEQ ID NO: 176 - DNA, 20 bp, Homo sapiens
gggccggggccgggaccgcg

SEQ ID NO: 177 - DNA, 20 bp, Homo sapiens
aagtgcgtgtcgccgggggc

SEQ ID NO: 178 - DNA, 20 bp, Homo sapiens
ctccggctggccccttctgt

SEQ ID NO: 179 - DNA, 20 bp, Homo sapiens
ggcggcgccgcgccgcatgc

SEQ ID NO: 180 - DNA, 20 bp, Homo sapiens
agtgcgtgtcgccgggggcc

SEQ ID NO: 181 - DNA, 20 bp, Homo sapiens
tgtgcggaagtgcgtgtcgc

SEQ ID NO: 182 - DNA, 20 bp, Homo sapiens
gtgtcgccgggggccggggc

SEQ ID NO: 183 - DNA, 20 bp, Homo sapiens
tgtcgccgggggccggggcc

SEQ ID NO: 184 - DNA, 20 bp, Homo sapiens
gcggtcccggccccggcccc

SEQ ID NO: 185 - DNA, 20 bp, Homo sapiens
cgcgggggccgcgggcgagg

SEQ ID NO: 186 - DNA, 20 bp, Homo sapiens
cgcgggcgaggaggaggaag

SEQ ID NO: 187 - DNA, 20 bp, Homo sapiens
gggcgaggaggaggaagagg

SEQ ID NO: 188 - DNA, 20 bp, Mus musculus
gaagtgcacgcggatgctcg

SEQ ID NO: 189 - DNA, 20 bp, Mus musculus
agtgctccagcagctcgaaa

SEQ ID NO: 190 - DNA, 20 bp, Mus musculus
gccggccgcttccacttgga

SEQ ID NO: 191 - DNA, 20 bp, Mus musculus
gctgtgtcgccagcgcatcg

SEQ ID NO: 192 - DNA, 20 bp, Mus musculus
gcgactgtcgcgcaccaaga

SEQ ID NO: 193 - DNA, 20 bp, Mus musculus
gcgtgcacggggcgcacgag

SEQ ID NO: 194 - DNA, 20 bp, Mus musculus
tcacggagtaccgggttaag

SEQ ID NO: 195 - DNA, 20 bp, Mus musculus
ggacgcctgcggcttctatt

SEQ ID NO: 196 - DNA, 20 bp, Mus musculus
gcgcgaagaagcagttccgt

SEQ ID NO: 197 - DNA, 20 bp, Mus musculus
gctcagcgtgaagatggctt

SEQ ID NO: 198 - DNA, 20 bp, Mus musculus
cgagcccgtgggcaccttct

SEQ ID NO: 199 - DNA, 20 bp, Mus musculus
atccgcgtgcacttccaggc

SEQ ID NO: 200 - DNA, 20 bp, Mus musculus
cgccaggttctcgcgaccca

SEQ ID NO: 201 - DNA, 20 bp, Homo sapiens
ccatgcccaccaccatcgag

SEQ ID NO: 202 - DNA, 20 bp, Homo sapiens
tctacggaaacgtattcgag

SEQ ID NO: 203 - DNA, 20 bp, Homo sapiens
tttagtatattgagaacttg

SEQ ID NO: 204 - DNA, 20 bp, Homo sapiens
gcactacagtggatcaccgc

SEQ ID NO: 205 - DNA, 20 bp, Homo sapiens
tgtcatgctgaaccgcattg

SEQ ID NO: 206 - DNA, 20 bp, Homo sapiens
ggaaacttggccactctatg

SEQ ID NO: 207 - DNA, 20 bp, Homo sapiens
gtatttgaaattattaatgc

SEQ ID NO: 208 - DNA, 20 bp, Homo sapiens
cagtttagttgacatagaag

SEQ ID NO: 209 - DNA, 20 bp, Homo sapiens
gggtctgaataagacccatt

SEQ ID NO: 210 - DNA, 20 bp, Homo sapiens
ccatgactatcctcatagag

SEQ ID NO: 211 - DNA, 20 bp, Homo sapiens
ccatgactatcctcatagag

SEQ ID NO: 212 - DNA, 20 bp, Homo sapiens
ctcttcgaactcccgctcga

SEQ ID NO: 213 - DNA, 20 bp, Homo sapiens
gaaccctgaccatgggcctg

SEQ ID NO: 214 - DNA, 20 bp, Homo sapiens
gctccttgaaccctgaccat

SEQ ID NO: 215 - DNA, 20 bp, Homo sapiens
agttggatactcagcgtcgc

SEQ ID NO: 216 - DNA, 20 bp, Homo sapiens
ccgctcgatggtggtgggca

SEQ ID NO: 217 - DNA, 20 bp, Homo sapiens
cagaaatggcagcatgtgtt

SEQ ID NO: 218 - DNA, 20 bp, Homo sapiens
gcactacagtggatcaccgc

SEQ ID NO: 219 - DNA, 20 bp, Homo sapiens
ggtagacacttgtcttgttt

SEQ ID NO: 220 - DNA, 20 bp, Homo sapiens
tggcagcatgtgttaggaag

SEQ ID NO: 221 - DNA, 20 bp, Homo sapiens
aggcccatggtcagggttca

SEQ ID NO: 222 - DNA, 20 bp, Homo sapiens
gttcagcatgacaactgctt

SEQ ID NO: 223 - DNA, 20 bp, Homo sapiens
caatggaggagaacagtgag

SEQ ID NO: 224 - DNA, 20 bp, Homo sapiens
ctcttctatgtcaactaaac

SEQ ID NO: 225 - DNA, 20 bp, Homo sapiens
agtggatcaccgcaggccca

SEQ ID NO: 226 - DNA, 20 bp, Homo sapiens
ctgacaggtgaccgatgtac

SEQ ID NO: 227 - DNA, 20 bp, Homo sapiens
aactcccgctcgatggtggt

SEQ ID NO: 228 - DNA, 20 bp, Homo sapiens
gtctccctgatccatccagt

SEQ ID NO: 229 - DNA, 20 bp, Homo sapiens
tagaggaaagtcctgtacat

SEQ ID NO: 230 - DNA, 20 bp, Homo sapiens
atgtatggaaaggatggtaa

SEQ ID NO: 231 - DNA, 20 bp, Homo sapiens
gcccaatgcctgcactacag

SEQ ID NO: 232 - DNA, 20 bp, Homo sapiens
cgagcgggagttcgaagagt

SEQ ID NO: 233 - DNA, 20 bp, Homo sapiens
tcaccgcaggcccatggtca

SEQ ID NO: 234 - DNA, 20 bp, Homo sapiens
cagtttagttgacatagaag

SEQ ID NO: 235 - DNA, 20 bp, Homo sapiens
ccatgcccaccaccatcgag

SEQ ID NO: 236 - DNA, 20 bp, Homo sapiens
gccaaaccataagccagaaa

SEQ ID NO: 237 - DNA, 20 bp, Homo sapiens
ccgattctttctccacaatg

SEQ ID NO: 238 - DNA, 20 bp, Homo sapiens
ttcgaactcccgctcgatgg

SEQ ID NO: 239 - DNA, 20 bp, Homo sapiens
agtgcaggcattgggcgctc

SEQ ID NO: 240 - DNA, 20 bp, Homo sapiens
ggaaacttggccactctatg

SEQ ID NO: 241 - DNA, 20 bp, Homo sapiens
atccactgtagtgcaggcat

SEQ ID NO: 242 - DNA, 20 bp, Homo sapiens
cactctatgaggatagtcat

SEQ ID NO: 243 - DNA, 20 bp, Homo sapiens
ccactctatgaggatagtca

SEQ ID NO: 244 - DNA, 20 bp, Homo sapiens
tccactgtagtgcaggcatt

SEQ ID NO: 245 - DNA, 20 bp, Homo sapiens
aagttctttccatcgtttct

SEQ ID NO: 246 - DNA, 20 bp, Homo sapiens
tcgctggcagccgctgtact

SEQ ID NO: 247 - DNA, 20 bp, Homo sapiens
gaactcccgctcgatggtgg

SEQ ID NO: 248 - DNA, 20 bp, Homo sapiens
aggatggtaaaggcaccaac

SEQ ID NO: 249 - DNA, 20 bp, Homo sapiens
aaagggagattctagtatac

SEQ ID NO: 250 - DNA, 20 bp, Homo sapiens
agaatttaggatgtatggaa

SEQ ID NO: 251 - DNA, 20 bp, Homo sapiens
gggtctgaataagacccatt

SEQ ID NO: 252 - DNA, 20 bp, Homo sapiens
ggcaccaactggatggatca

SEQ ID NO: 253 - DNA, 20 bp, Homo sapiens
ctctaaaatgcaagatacaa

SEQ ID NO: 254 - DNA, 20 bp, Homo sapiens
gtatttgaaattattaatgc

SEQ ID NO: 255 - DNA, 20 bp, Homo sapiens
cctttcttgcagatggaaaa

SEQ ID NO: 256 - DNA, 20 bp, Homo sapiens
ctgcaccttctgagctgtgg

SEQ ID NO: 257 - DNA, 20 bp, Homo sapiens
atgctgccatttctggctta

SEQ ID NO: 258 - DNA, 20 bp, Homo sapiens
tttctttaaacagcatctct

SEQ ID NO: 259 - DNA, 20 bp, Homo sapiens
agacatggaatgcagaatgc

SEQ ID NO: 260 - DNA, 20 bp, Homo sapiens
aggcaccaactggatggatc

SEQ ID NO: 261 - DNA, 20 bp, Homo sapiens
taatgactgaaaaatacaat

SEQ ID NO: 262 - DNA, 20 bp, Homo sapiens
gaatgcagaatgcaggaaat

SEQ ID NO: 263 - DNA, 20 bp, Homo sapiens
tttaggatgtatggaaagga

SEQ ID NO: 264 - DNA, 20 bp, Homo sapiens
ctaacacatgctgccatttc

SEQ ID NO: 265 - DNA, 20 bp, Homo sapiens
tcatacatggctataataga

SEQ ID NO: 266 - DNA, 20 bp, Homo sapiens
acgatggaaagaactttcta

SEQ ID NO: 267 - DNA, 20 bp, Homo sapiens
acgtattcgagaggacagaa

SEQ ID NO: 268 - DNA, 20 bp, Homo sapiens
gcggtgatccactgtagtgc

SEQ ID NO: 269 - DNA, 20 bp, Homo sapiens
tattaatgctggatgttaaa

SEQ ID NO: 270 - DNA, 20 bp, Homo sapiens
gagatgctgtttaaagaaac

SEQ ID NO: 271 - DNA, 20 bp, Homo sapiens
cagcaagaatttaggatgta

SEQ ID NO: 272 - DNA, 20 bp, Homo sapiens
ttgacatagaagaggcacaa

SEQ ID NO: 273 - DNA, 20 bp, Homo sapiens
gattcagggactccaaaatc

SEQ ID NO: 274 - DNA, 20 bp, Homo sapiens
ctcactttcattatactacc

SEQ ID NO: 275 - DNA, 20 bp, Homo sapiens
tttagtatattgagaacttg

SEQ ID NO: 276 - DNA, 20 bp, Homo sapiens
agggactccaaaatctggcc

SEQ ID NO: 277 - DNA, 20 bp, Homo sapiens
aggttaaatgtgcacagtac

SEQ ID NO: 278 - DNA, 20 bp, Homo sapiens
atcaccgcaggcccatggtc

SEQ ID NO: 279 - DNA, 20 bp, Homo sapiens
agcatctcttggtcatctgt

SEQ ID NO: 280 - DNA, 20 bp, Homo sapiens
gaaggagcaaaatgtataaa

SEQ ID NO: 281 - DNA, 20 bp, Homo sapiens
gccatttctggcttatggtt

SEQ ID NO: 282 - DNA, 20 bp, Homo sapiens
ctggatggatcagggagaca

SEQ ID NO: 283 - DNA, 20 bp, Homo sapiens
aaatacaatgggaacagaat

SEQ ID NO: 284 - DNA, 20 bp, Homo sapiens
ataatgactgaaaaatacaa

SEQ ID NO: 285 - DNA, 20 bp, Homo sapiens
catgcccaccaccatcgagc

SEQ ID NO: 286 - DNA, 20 bp, Homo sapiens
aacatgagaaaataccgaat

SEQ ID NO: 287 - DNA, 20 bp, Homo sapiens
agaaatgaagctggtgattc

SEQ ID NO: 288 - DNA, 20 bp, Homo sapiens
ccgcattgtggagaaagaat

SEQ ID NO: 289 - DNA, 20 bp, Homo sapiens
gaaatgaagctggtgattca

SEQ ID NO: 290 - DNA, 20 bp, Homo sapiens
ttgtttaaagtgagagaatc

SEQ ID NO: 291 - DNA, 20 bp, Homo sapiens
ccgcgactcaccaagtacag

SEQ ID NO: 292 - DNA, 20 bp, Homo sapiens
gaacatgagaaaataccgaa

SEQ ID NO: 293 - DNA, 20 bp, Homo sapiens
tatactacctggccagattt

SEQ ID NO: 294 - DNA, 20 bp, Homo sapiens
tatgagaatctcagttgatc

SEQ ID NO: 295 - DNA, 20 bp, Homo sapiens
tcaactgagattctcataca

SEQ ID NO: 296 - DNA, 20 bp, Homo sapiens
tgagaatctcagttgatctg

SEQ ID NO: 297 - DNA, 20 bp, Homo sapiens
atgagaatctcagttgatct

SEQ ID NO: 298 - DNA, 20 bp, Homo sapiens
tggtaaaggcaccaactgga

SEQ ID NO: 299 - DNA, 20 bp, Homo sapiens
tgtcatgctgaaccgcattg

SEQ ID NO: 300 - DNA, 20 bp, Homo sapiens
tttggtgaatgatcaaaggc

SEQ ID NO: 301 - DNA, 20 bp, Homo sapiens
atgaaagtgagatattgttc

SEQ ID NO: 302 - DNA, 20 bp, Homo sapiens
tatttcctcatagtgctcta

SEQ ID NO: 303 - DNA, 20 bp, Homo sapiens
agaaggagcaaaatgtataa

SEQ ID NO: 304 - DNA, 20 bp, Homo sapiens
tttgtttggtgaatgatcaa

SEQ ID NO: 305 - DNA, 20 bp, Homo sapiens
tctacggaaacgtattcgag

SEQ ID NO: 306 - DNA, 20 bp, Homo sapiens
aaaggccaccacagctcaga

SEQ ID NO: 307 - DNA, 20 bp, Homo sapiens
aggtgcagcagatgaaacag

SEQ ID NO: 308 - DNA, 20 bp, Homo sapiens
ggctccttgaaccctgacca

SEQ ID NO: 309 - DNA, 20 bp, Homo sapiens
aaggagttacatcttaacac

SEQ ID NO: 310 - DNA, 20 bp, Homo sapiens
taaaatgcaagatacaatgg

SEQ ID NO: 311 - DNA, 20 bp, Homo sapiens
acaagtgtctaccagagaga

SEQ ID NO: 312 - DNA, 20 bp, Homo sapiens
gcgctctggcaccttctctc

SEQ ID NO: 313 - DNA, 20 bp, Homo sapiens
ctgctgcaccttctgagctg

SEQ ID NO: 314 - DNA, 20 bp, Homo sapiens
tcttccctacctagaaacga

SEQ ID NO: 315 - DNA, 20 bp, Mus musculus
aatctggccaggtggtataa

SEQ ID NO: 316 - DNA, 20 bp, Mus musculus
aatatgagaaagtatcgaat

SEQ ID NO: 317 - DNA, 20 bp, Mus musculus
atcactgcaggtccatggtc

SEQ ID NO: 318 - DNA, 20 bp, Mus musculus
atgtgcacagtactggccaa

SEQ ID NO: 319 - DNA, 20 bp, Mus musculus
ggcagcatgtgttcggaagt

SEQ ID NO: 320 - DNA, 20 bp, Mus musculus
aagaagtttagaaatgaagc

SEQ ID NO: 321 - DNA, 20 bp, Mus musculus
gccacaccatgagccagaaa

SEQ ID NO: 322 - DNA, 20 bp, Mus musculus
cctttcttgcagatggaaaa

SEQ ID NO: 323 - DNA, 20 bp, Mus musculus
gtactttgctccttctatta

SEQ ID NO: 324 - DNA, 20 bp, Mus musculus
agaaatgaagctggtgactc

SEQ ID NO: 325 - DNA, 20 bp, Mus musculus
gtttagcatgacaactgctt

SEQ ID NO: 326 - DNA, 20 bp, Mus musculus
gcccgatgcccgcactgcaa

SEQ ID NO: 327 - DNA, 20 bp, Mus musculus
tgacagagaaatggtgttta

SEQ ID NO: 328 - DNA, 25 bp, Mus musculus
ccuggacaacuuccuucguaagaaa

SEQ ID NO: 329 - RNA, 19 nt, Homo sapiens
gugucccuauggaaggaaa

SEQ ID NO: 330 - RNA, 19 nt, Homo sapiens
caacuuccuucguaagaaa

SEQ ID NO: 331 - DNA, 21 bp, Homo sapiens
agcgaggccacacagatatta

SEQ ID NO: 332 - DNA, 21 bp, Homo sapiens
gctatgatgaccgcttcattg

SEQ ID NO: 333 - DNA, 21 bp, Homo sapiens
tggtctgagccgtacccatta

SEQ ID NO: 334 - DNA, 21 bp, Homo sapiens
ctgtgtacagaggcgagattt

SEQ ID NO: 335 - DNA, 25 bp, Homo sapiens
cctggacaacttccttcgtaagaaa

SEQ ID NO: 336 - DNA, 19 bp, Homo sapiens
gtgtccctatggaaggaaa

SEQ ID NO: 337 - DNA, 19 bp, Homo sapiens
caacttccttcgtaagaaa

SEQ ID NO: 338 - DNA, 20 bp, Homo sapiens
aagctggcctacgagtctga

SEQ ID NO: 339 - DNA, 20 bp, Homo sapiens
gcgggactagagggagctga

SEQ ID NO: 340 - DNA, 20 bp, Homo sapiens
cagctccctctagtcccgcg

SEQ ID NO: 341 - DNA, 20 bp, Homo sapiens
caggacgctgtggatctccg

SEQ ID NO: 342 - DNA, 20 bp, Homo sapiens
aacatacttgtcattgacga

SEQ ID NO: 343 - DNA, 20 bp, Homo sapiens
ctcacctgtgatgggcacgt

SEQ ID NO: 344 - DNA, 20 bp, Homo sapiens
aacacgggacagccaccgag

SEQ ID NO: 345 - DNA, 20 bp, Homo sapiens
cgacagattcattgtgaagc

SEQ ID NO: 346 - DNA, 20 bp, Homo sapiens
acaccatcacgacgcgtggg

SEQ ID NO: 347 - DNA, 20 bp, Homo sapiens
tcccagccatgggaacaagg

SEQ ID NO: 348 - DNA, 20 bp, Homo sapiens
ggagtggaagcgcttcatcg

SEQ ID NO: 349 - DNA, 20 bp, Homo sapiens
ttaggggtgccaccaccccg

SEQ ID NO: 350 - DNA, 20 bp, Homo sapiens
gacacataccgtgacctcca

SEQ ID NO: 351 - DNA, 20 bp, Homo sapiens
ccggcccagtgggtcatcag

SEQ ID NO: 352 - DNA, 20 bp, Homo sapiens
cctggaactgcagatgaagg

SEQ ID NO: 353 - DNA, 20 bp, Homo sapiens
gtcctctccctcccagccat

SEQ ID NO: 354 - DNA, 20 bp, Homo sapiens
tccccagggtcccgccaaga

SEQ ID NO: 355 - DNA, 20 bp, Homo sapiens
agtgagcagtgcagcctgga

SEQ ID NO: 356 - DNA, 20 bp, Homo sapiens
tgtcctctccctcccagcca

SEQ ID NO: 357 - DNA, 20 bp, Homo sapiens
ctggactgggatgaaggtga

SEQ ID NO: 358 - DNA, 20 bp, Homo sapiens
ggggtgggcccggctcacca

SEQ ID NO: 359 - DNA, 20 bp, Homo sapiens
caccaccccgcgggactaga

SEQ ID NO: 360 - DNA, 20 bp, Homo sapiens
ctgctgccactgcccccgct

SEQ ID NO: 361 - DNA, 20 bp, Homo sapiens
cggcccgacgtgcccatcac

SEQ ID NO: 362 - DNA, 20 bp, Homo sapiens
cactgcccccgctaggtgcg

SEQ ID NO: 363 - DNA, 20 bp, Homo sapiens
atacacgctggcctgctcct

SEQ ID NO: 364 - DNA, 20 bp, Homo sapiens
caaacactgtgatgtctgtg

SEQ ID NO: 365 - DNA, 20 bp, Homo sapiens
gcgggaccctggggatgcct

SEQ ID NO: 366 - DNA, 20 bp, Homo sapiens
gcgggagcgccagacctcac

SEQ ID NO: 367 - DNA, 20 bp, Homo sapiens
aggacaggcttctctccaca

SEQ ID NO: 368 - DNA, 20 bp, Homo sapiens
gcagacaccaacacggtgct

SEQ ID NO: 369 - DNA, 20 bp, Homo sapiens
ccaccaccccgcgggactag

SEQ ID NO: 370 - DNA, 20 bp, Homo sapiens
atccccagggtcccgccaag

SEQ ID NO: 371 - DNA, 20 bp, Homo sapiens
cctggaggaaggagcagcct

SEQ ID NO: 372 - DNA, 20 bp, Homo sapiens
agagccagatgtcggaactt

SEQ ID NO: 373 - DNA, 20 bp, Homo sapiens
atgacccactgggccggcac

SEQ ID NO: 374 - DNA, 20 bp, Homo sapiens
gcagctttgggcccacagac

SEQ ID NO: 375 - DNA, 20 bp, Homo sapiens
actctctgttagcagagagc

SEQ ID NO: 376 - DNA, 20 bp, Homo sapiens
ccaggaaggaaatgcaccta

SEQ ID NO: 377 - DNA, 20 bp, Homo sapiens
aggcaccactcacctgtgat

SEQ ID NO: 378 - DNA, 20 bp, Homo sapiens
ctgggcccgtgccggcccag

SEQ ID NO: 379 - DNA, 20 bp, Homo sapiens
cagccagctgctgggggtcc

SEQ ID NO: 380 - DNA, 20 bp, Homo sapiens
tccactcctgccgctcgcct

SEQ ID NO: 381 - DNA, 20 bp, Homo sapiens
cgtccaggcagacaccaaca

SEQ ID NO: 382 - DNA, 20 bp, Homo sapiens
cccacccacatcagtccttc

SEQ ID NO: 383 - DNA, 20 bp, Homo sapiens
gccagctcttgacccggcct

SEQ ID NO: 384 - DNA, 20 bp, Homo sapiens
ctgccctccttttcctcttc

SEQ ID NO: 385 - DNA, 20 bp, Homo sapiens
ccagccccaccatgagtctg

SEQ ID NO: 386 - DNA, 20 bp, Homo sapiens
gccgattcttccacccagag

SEQ ID NO: 387 - DNA, 20 bp, Homo sapiens
ctcccagaagaggaaaagga

SEQ ID NO: 388 - DNA, 20 bp, Homo sapiens
gtggggcagggcaggcagcc

SEQ ID NO: 389 - DNA, 20 bp, Homo sapiens
gggtcaagagctggccgctg

SEQ ID NO: 390 - DNA, 20 bp, Homo sapiens
atgccccctgatgacccact

SEQ ID NO: 391 - DNA, 20 bp, Homo sapiens
agccttctctgcctttggcc

SEQ ID NO: 392 - DNA, 20 bp, Homo sapiens
ctctgcctttggccgggcca

SEQ ID NO: 393 - DNA, 20 bp, Homo sapiens
ggaacccagcctgccctccc

SEQ ID NO: 394 - DNA, 20 bp, Homo sapiens
ggcaggagcctcgcacctag

SEQ ID NO: 395 - DNA, 20 bp, Homo sapiens
tcccagaccagcacatcctg

SEQ ID NO: 396 - DNA, 20 bp, Homo sapiens
gtgagcagtgcagcctggat

SEQ ID NO: 397 - DNA, 20 bp, Homo sapiens
gagccagatgtcggaacttt

SEQ ID NO: 398 - DNA, 20 bp, Homo sapiens
ggccgatggcaagccttgct

SEQ ID NO: 399 - DNA, 20 bp, Homo sapiens
aggagcctcgcacctagcgg

SEQ ID NO: 400 - DNA, 20 bp, Homo sapiens
aggtccccaagaggaaaaca

SEQ ID NO: 401 - DNA, 20 bp, Homo sapiens
cgctgaggaggcctcggccc

SEQ ID NO: 402 - DNA, 20 bp, Homo sapiens
gaggacagccacagccgtca

SEQ ID NO: 403 - DNA, 20 bp, Homo sapiens
cagccccaccatgagtctgt

SEQ ID NO: 404 - DNA, 20 bp, Homo sapiens
accccccagagccccaagca

SEQ ID NO: 405 - DNA, 20 bp, Homo sapiens
gaggcaccactcacctgtga

SEQ ID NO: 406 - DNA, 20 bp, Homo sapiens
ccaagaggaaaacagggcac

SEQ ID NO: 407 - DNA, 20 bp, Homo sapiens
gtacgtctcccaggattgcc

SEQ ID NO: 408 - DNA, 20 bp, Homo sapiens
cacagcctccaccaggtgcg

SEQ ID NO: 409 - DNA, 20 bp, Homo sapiens
gatctcggcagccagctgct

SEQ ID NO: 410 - DNA, 20 bp, Homo sapiens
cagccttctctgcctttggc

SEQ ID NO: 411 - DNA, 20 bp, Homo sapiens
cagaagtgacacttacctca

SEQ ID NO: 412 - DNA, 20 bp, Homo sapiens
gctggccgctgaggaggcct

SEQ ID NO: 413 - DNA, 20 bp, Homo sapiens
cagctccctctagtcccgcg

SEQ ID NO: 414 - DNA, 20 bp, Homo sapiens
cggggtgggcccggctcacc

SEQ ID NO: 415 - DNA, 20 bp, Homo sapiens
gacacataccgtgacctcca

SEQ ID NO: 416 - DNA, 20 bp, Homo sapiens
caggaaggaaatgcacctat

SEQ ID NO: 417 - DNA, 20 bp, Homo sapiens
agtggccagcacccatggcc

SEQ ID NO: 418 - DNA, 20 bp, Homo sapiens
ctctcctattcttcccagca

SEQ ID NO: 419 - DNA, 20 bp, Homo sapiens
gcccgagtccaggcaatcct

SEQ ID NO: 420 - DNA, 20 bp, Homo sapiens
caccttcatctgcagttcca

SEQ ID NO: 421 - DNA, 20 bp, Homo sapiens
ggcacaggcagacaggtgag

SEQ ID NO: 422 - DNA, 20 bp, Homo sapiens
agcacccatggcccggccaa

SEQ ID NO: 423 - DNA, 20 bp, Homo sapiens
ccacaggcagcttactcact

SEQ ID NO: 424 - DNA, 20 bp, Homo sapiens
ttcctgtgctccaaagtgag

SEQ ID NO: 425 - DNA, 20 bp, Homo sapiens
accgcagccttctctgcctt

SEQ ID NO: 426 - DNA, 20 bp, Homo sapiens
gggagccaatgcccgagtcc

SEQ ID NO: 427 - DNA, 20 bp, Homo sapiens
ttcccagcaaggcttgccat

SEQ ID NO: 428 - DNA, 20 bp, Homo sapiens
agccagatgtcggaactttg

SEQ ID NO: 429 - DNA, 20 bp, Homo sapiens
tacacgggctacagtcccta

SEQ ID NO: 430 - DNA, 20 bp, Homo sapiens
tctgtgttagaccctcttgg

SEQ ID NO: 431 - DNA, 20 bp, Homo sapiens
aagctgcccccagcgctctg

SEQ ID NO: 432 - DNA, 20 bp, Homo sapiens
ctttggggggttcgaggagg

SEQ ID NO: 433 - DNA, 20 bp, Homo sapiens
gggccgatggcaagccttgc

SEQ ID NO: 434 - DNA, 20 bp, Homo sapiens
cacaggcagcttactcactg

SEQ ID NO: 435 - DNA, 20 bp, Homo sapiens
cccagaccagcacatcctgc

SEQ ID NO: 436 - DNA, 20 bp, Homo sapiens
aggctgggttccataccata

SEQ ID NO: 437 - DNA, 20 bp, Homo sapiens
ggacttctaattgctgagaa

SEQ ID NO: 438 - DNA, 20 bp, Homo sapiens
ctcaaattcccacagactca

SEQ ID NO: 439 - DNA, 20 bp, Homo sapiens
aaaacagggcacaggcagac

SEQ ID NO: 440 - DNA, 20 bp, Homo sapiens
ccagatgtcggaactttggg

SEQ ID NO: 441 - DNA, 20 bp, Homo sapiens
ctccctctagtcccgcgggg

SEQ ID NO: 442 - DNA, 20 bp, Homo sapiens
agcccccagtgcagagccca

SEQ ID NO: 443 - DNA, 20 bp, Homo sapiens
cctggacgcccagcttctgc

SEQ ID NO: 444 - DNA, 20 bp, Homo sapiens
caggggctggcaggagcccg

SEQ ID NO: 445 - DNA, 20 bp, Homo sapiens
ccttgttcccatggctggga

SEQ ID NO: 446 - DNA, 20 bp, Homo sapiens
ctcatctgccacagagcgct

SEQ ID NO: 447 - DNA, 20 bp, Homo sapiens
ggcagacaccaacacggtgc

SEQ ID NO: 448 - DNA, 20 bp, Homo sapiens
tccctcttgattcctcttcc

SEQ ID NO: 449 - DNA, 20 bp, Homo sapiens
ccctcccagccatgggaaca

SEQ ID NO: 450 - DNA, 20 bp, Homo sapiens
gcgtaagaagccactcactt

SEQ ID NO: 451 - DNA, 20 bp, Homo sapiens
tgtgtttcccccgcacctgg

SEQ ID NO: 452 - DNA, 20 bp, Homo sapiens
ctgagaccagtggtcatcga

SEQ ID NO: 453 - DNA, 20 bp, Homo sapiens
gggcagcgacctgagaccag

SEQ ID NO: 454 - DNA, 20 bp, Homo sapiens
agcaattagaagtccctgca

SEQ ID NO: 455 - DNA, 20 bp, Homo sapiens
tgggtgagctggtgaaacac

SEQ ID NO: 456 - DNA, 20 bp, Homo sapiens
ctgttagcagagagctggac

SEQ ID NO: 457 - DNA, 20 bp, Homo sapiens
cccctgatgacccactgggc

SEQ ID NO: 458 - DNA, 20 bp, Homo sapiens
gttcacaccatcacgacgcg

SEQ ID NO: 459 - DNA, 20 bp, Homo sapiens
tgtccaggctgggcccgtgc

SEQ ID NO: 460 - DNA, 20 bp, Homo sapiens
acacagacctatgccccatc

SEQ ID NO: 461 - DNA, 20 bp, Homo sapiens
ggctgcctgccctgccccac

SEQ ID NO: 462 - DNA, 20 bp, Homo sapiens
ccataggtgcatttccttcc

SEQ ID NO: 463 - DNA, 20 bp, Homo sapiens
caggctgggttccataccat

SEQ ID NO: 464 - DNA, 20 bp, Homo sapiens
gccccatcacagcctccacc

SEQ ID NO: 465 - DNA, 20 bp, Homo sapiens
tgccctccttttcctcttct

SEQ ID NO: 466 - DNA, 20 bp, Homo sapiens
gccagatgtcggaactttgg

SEQ ID NO: 467 - DNA, 20 bp, Homo sapiens
caggcagacaggtgagagga

SEQ ID NO: 468 - DNA, 20 bp, Homo sapiens
ccaggagtctgagctatgag

SEQ ID NO: 469 - DNA, 20 bp, Homo sapiens
gctccaggttgggagcctta

SEQ ID NO: 470 - DNA, 20 bp, Homo sapiens
ctcacctgtgatgggcacgt

SEQ ID NO: 471 - DNA, 20 bp, Homo sapiens
agctggcctacgagtctgac

SEQ ID NO: 472 - DNA, 20 bp, Homo sapiens
gtgggtgggggcagtgggta

SEQ ID NO: 473 - DNA, 20 bp, Homo sapiens
catctgcagttccagggccg

SEQ ID NO: 474 - DNA, 20 bp, Homo sapiens
gatgacccactgggccggca

SEQ ID NO: 475 - DNA, 20 bp, Homo sapiens
tgacctccaaggcgagcggc

SEQ ID NO: 476 - DNA, 20 bp, Homo sapiens
ggatctcggcagccagctgc

SEQ ID NO: 477 - DNA, 20 bp, Homo sapiens
tccttttcctcttctgggag

SEQ ID NO: 478 - DNA, 20 bp, Homo sapiens
cacgacgcgtgggtggcaag

SEQ ID NO: 479 - DNA, 20 bp, Homo sapiens
ttcacaccatcacgacgcgt

SEQ ID NO: 480 - DNA, 20 bp, Homo sapiens
gcaggagcctcgcacctagc

SEQ ID NO: 481 - DNA, 20 bp, Homo sapiens
cacccctaaggctcccaacc

SEQ ID NO: 482 - DNA, 20 bp, Homo sapiens
ttgtccttgcttggggctct

SEQ ID NO: 483 - DNA, 20 bp, Homo sapiens
caggacaggcttctctccac

SEQ ID NO: 484 - DNA, 20 bp, Homo sapiens
cacctggtggaggctgtgat

SEQ ID NO: 485 - DNA, 20 bp, Homo sapiens
cgtctgtgggagccagtctg

SEQ ID NO: 486 - DNA, 20 bp, Homo sapiens
ccccccaaagttccgacatc

SEQ ID NO: 487 - DNA, 20 bp, Homo sapiens
aggcagcctggccaaggagc

SEQ ID NO: 488 - DNA, 20 bp, Homo sapiens
tctgcctttggccgggccat

SEQ ID NO: 489 - DNA, 20 bp, Homo sapiens
ggacaggcttctctccacag

SEQ ID NO: 490 - DNA, 20 bp, Homo sapiens
acgtgcccatcacaggtgag

SEQ ID NO: 491 - DNA, 20 bp, Homo sapiens
agagagtgagcagtgcagcc

SEQ ID NO: 492 - DNA, 20 bp, Homo sapiens
cgcaggaagttgtccaggct

SEQ ID NO: 493 - DNA, 20 bp, Homo sapiens
ggctgggagctcagatccat

SEQ ID NO: 494 - DNA, 20 bp, Homo sapiens
cagctcacccagcaccgtgt

SEQ ID NO: 495 - DNA, 20 bp, Homo sapiens
ccagcacatcctgcgggaac

SEQ ID NO: 496 - DNA, 20 bp, Homo sapiens
gacctccttgttcccatggc

SEQ ID NO: 497 - DNA, 20 bp, Homo sapiens
ggggttcgaggaggaggccc

SEQ ID NO: 498 - DNA, 20 bp, Homo sapiens
cagagaaggctgcggtggct

SEQ ID NO: 499 - DNA, 20 bp, Homo sapiens
gggagtgagtccagcgtctg

SEQ ID NO: 500 - DNA, 20 bp, Homo sapiens
caggagcctcgcacctagcg

SEQ ID NO: 501 - DNA, 20 bp, Homo sapiens
ggaggaggccctggtgagcc

SEQ ID NO: 502 - DNA, 20 bp, Homo sapiens
caagcaaggacaaaaatggc

SEQ ID NO: 503 - DNA, 20 bp, Homo sapiens
cgtcagggcaccccaaggcc

SEQ ID NO: 504 - DNA, 20 bp, Homo sapiens
gctggcagtgaactggtttc

SEQ ID NO: 505 - DNA, 20 bp, Homo sapiens
acctccttgttcccatggct

SEQ ID NO: 506 - DNA, 20 bp, Homo sapiens
tcccgcaggatgtgctggtc

SEQ ID NO: 507 - DNA, 20 bp, Homo sapiens
agggactgtagcccgtgtaa

SEQ ID NO: 508 - DNA, 20 bp, Homo sapiens
ccagtactctcgaggtggaa

SEQ ID NO: 509 - DNA, 20 bp, Homo sapiens
aattcccacagactcatggt

SEQ ID NO: 510 - DNA, 20 bp, Homo sapiens
cccaccccgagccccttaca

SEQ ID NO: 511 - DNA, 20 bp, Homo sapiens
gtgcatttccttcctggaag

SEQ ID NO: 512 - DNA, 20 bp, Homo sapiens
tcagcggccagctcttgacc

SEQ ID NO: 513 - DNA, 20 bp, Homo sapiens
ggcccggccaaaggcagaga

SEQ ID NO: 514 - DNA, 20 bp, Homo sapiens
acagagcgctgggggcagct

SEQ ID NO: 515 - DNA, 20 bp, Homo sapiens
tcttgattcctcttccagga

SEQ ID NO: 516 - DNA, 20 bp, Homo sapiens
gcaaggacaaaaatggccgg

SEQ ID NO: 517 - DNA, 20 bp, Homo sapiens
cagggcaggcagcctggcca

SEQ ID NO: 518 - DNA, 20 bp, Homo sapiens
atctcggcagccagctgctg

SEQ ID NO: 519 - DNA, 20 bp, Homo sapiens
cccgcaggatgtgctggtct

SEQ ID NO: 520 - DNA, 20 bp, Homo sapiens
ggctccaggttgggagcctt

SEQ ID NO: 521 - DNA, 20 bp, Homo sapiens
caacacggtgctgggtgagc

SEQ ID NO: 522 - DNA, 20 bp, Homo sapiens
gcagccgtgtccctatggta

SEQ ID NO: 523 - DNA, 20 bp, Homo sapiens
tgtccttgcttggggctctg

SEQ ID NO: 524 - DNA, 20 bp, Homo sapiens
tcatggtggggctggcttcc

SEQ ID NO: 525 - DNA, 20 bp, Homo sapiens
gaagctgggctattcatcca

SEQ ID NO: 526 - DNA, 20 bp, Homo sapiens
gaccctcttggcgggaccct

SEQ ID NO: 527 - DNA, 20 bp, Homo sapiens
ggaaaggcagggggcgcggg

SEQ ID NO: 528 - DNA, 20 bp, Homo sapiens
aggtctgtgttagaccctct

SEQ ID NO: 529 - DNA, 20 bp, Homo sapiens
ctcagctccctctagtcccg

SEQ ID NO: 530 - DNA, 20 bp, Homo sapiens
tagggactgtagcccgtgta

SEQ ID NO: 531 - DNA, 20 bp, Homo sapiens
agggggcataaacctgcaga

SEQ ID NO: 532 - DNA, 20 bp, Homo sapiens
ctcccaggattgcctggact

SEQ ID NO: 533 - DNA, 20 bp, Homo sapiens
gggatgaaggtgaaggccgc

SEQ ID NO: 534 - DNA, 20 bp, Homo sapiens
tgcagagcccaggggctggc

SEQ ID NO: 535 - DNA, 20 bp, Homo sapiens
gaatcggcacttgatcccat

SEQ ID NO: 536 - DNA, 20 bp, Homo sapiens
ccgaggctgctccttcctcc

SEQ ID NO: 537 - DNA, 20 bp, Homo sapiens
ccagcttctgcaggacgctg

SEQ ID NO: 538 - DNA, 20 bp, Homo sapiens
gggccggcacgggcccagcc

SEQ ID NO: 539 - DNA, 20 bp, Homo sapiens
tgaggtctggcgctcccgct

SEQ ID NO: 540 - DNA, 20 bp, Homo sapiens
ttggggtgccctgacggctg

SEQ ID NO: 541 - DNA, 20 bp, Homo sapiens
actagagggagctgagggca

SEQ ID NO: 542 - DNA, 20 bp, Homo sapiens
ccagttcccgcaggatgtgc

SEQ ID NO: 543 - DNA, 20 bp, Homo sapiens
tatgccccctgatgacccac

SEQ ID NO: 544 - DNA, 20 bp, Homo sapiens
gtgagaggagagcattggca

SEQ ID NO: 545 - DNA, 20 bp, Homo sapiens
agcttactcactggggtgct

SEQ ID NO: 546 - DNA, 20 bp, Homo sapiens
atcacagcctccaccaggtg

SEQ ID NO: 547 - DNA, 20 bp, Homo sapiens
actgaagtggccagcaccca

SEQ ID NO: 548 - DNA, 20 bp, Homo sapiens
gccggcccagtgggtcatca

SEQ ID NO: 549 - DNA, 20 bp, Homo sapiens
cctgcagaagctgggcgtcc

SEQ ID NO: 550 - DNA, 20 bp, Homo sapiens
gcaccgtgttggtgtctgcc

SEQ ID NO: 551 - DNA, 20 bp, Homo sapiens
ggccctggaactgcagatga

SEQ ID NO: 552 - DNA, 20 bp, Homo sapiens
gtccttgcttggggctctgg

SEQ ID NO: 553 - DNA, 20 bp, Homo sapiens
ctccctggagagccagatgt

SEQ ID NO: 554 - DNA, 20 bp, Homo sapiens
aaattcccacagactcatgg

SEQ ID NO: 555 - DNA, 20 bp, Homo sapiens
tcatctgccacagagcgctg

SEQ ID NO: 556 - DNA, 20 bp, Homo sapiens
agtcggcagggacactgaag

SEQ ID NO: 557 - DNA, 20 bp, Homo sapiens
actctcgaggtggaaaggca

SEQ ID NO: 558 - DNA, 20 bp, Homo sapiens
cccagtgagtaagctgcctg

SEQ ID NO: 559 - DNA, 20 bp, Homo sapiens
agagggtgcaaagaactctc

SEQ ID NO: 560 - DNA, 20 bp, Homo sapiens
cacgatcccgtcagactcgt

SEQ ID NO: 561 - DNA, 20 bp, Homo sapiens
tctgcactgggggctcctga

SEQ ID NO: 562 - DNA, 20 bp, Homo sapiens
cagggggcataaacctgcag

SEQ ID NO: 563 - DNA, 20 bp, Homo sapiens
tgaggacagccacagccgtc

SEQ ID NO: 564 - DNA, 20 bp, Homo sapiens
gtttcccccgcacctggtgg

SEQ ID NO: 565 - DNA, 20 bp, Homo sapiens
ttaggggtgccaccaccccg

SEQ ID NO: 566 - DNA, 20 bp, Homo sapiens
actggggtgctgggacttgt

SEQ ID NO: 567 - DNA, 20 bp, Homo sapiens
ctcactcccgtacgtctccc

SEQ ID NO: 568 - DNA, 20 bp, Homo sapiens
aggggctggcaggagcccgt

SEQ ID NO: 569 - DNA, 20 bp, Homo sapiens
tccttgttcccatggctggg

SEQ ID NO: 570 - DNA, 20 bp, Homo sapiens
gccaaaggcagagaaggctg

SEQ ID NO: 571 - DNA, 20 bp, Homo sapiens
cacgggctcctgccagcccc

SEQ ID NO: 572 - DNA, 20 bp, Homo sapiens
ccacagcgtcctgcagaagc

SEQ ID NO: 573 - DNA, 20 bp, Homo sapiens
acgggctcctgccagcccct

SEQ ID NO: 574 - DNA, 20 bp, Homo sapiens
atgggagcaacgtggccatg

SEQ ID NO: 575 - DNA, 20 bp, Homo sapiens
cccaaggccgggtcaagagc

SEQ ID NO: 576 - DNA, 20 bp, Homo sapiens
aattgctgagaaggggccga

SEQ ID NO: 577 - DNA, 20 bp, Homo sapiens
gggcaggagtgaggagggcc

SEQ ID NO: 578 - DNA, 20 bp, Homo sapiens
ggcgggaccctggggatgcc

SEQ ID NO: 579 - DNA, 20 bp, Homo sapiens
ggggctggcaggagcccgtg

SEQ ID NO: 580 - DNA, 20 bp, Homo sapiens
ttccgacatctggctctcca

SEQ ID NO: 581 - DNA, 20 bp, Homo sapiens
gtgctgcccttgccagccac

SEQ ID NO: 582 - DNA, 20 bp, Homo sapiens
actcctgccgctcgccttgg

SEQ ID NO: 583 - DNA, 20 bp, Homo sapiens
gtggacttcttccggaagct

SEQ ID NO: 584 - DNA, 20 bp, Homo sapiens
ccagtgcagagcccaggggc

SEQ ID NO: 585 - DNA, 20 bp, Homo sapiens
ggggcagtggcagcagcttt

SEQ ID NO: 586 - DNA, 20 bp, Homo sapiens
gggactgtagcccgtgtaag

SEQ ID NO: 587 - DNA, 20 bp, Homo sapiens
ccacagactcatggtggggc

SEQ ID NO: 588 - DNA, 20 bp, Homo sapiens
aacacgggacagccaccgag

SEQ ID NO: 589 - DNA, 20 bp, Homo sapiens
gcaaagaactctctggaggt

SEQ ID NO: 590 - DNA, 20 bp, Homo sapiens
tgggcccgtgccggcccagt

SEQ ID NO: 591 - DNA, 20 bp, Homo sapiens
ctcctgccggggcatcctgc

SEQ ID NO: 592 - DNA, 20 bp, Homo sapiens
aggcagacaggtgagaggaa

SEQ ID NO: 593 - DNA, 20 bp, Homo sapiens
aggcaatcctgggagacgta

SEQ ID NO: 594 - DNA, 20 bp, Homo sapiens
tcagaccagtactctcgagg

SEQ ID NO: 595 - DNA, 20 bp, Homo sapiens
aacatacttgtcattgacga

SEQ ID NO: 596 - DNA, 20 bp, Homo sapiens
ggcagcttggccgctctggg

SEQ ID NO: 597 - DNA, 20 bp, Homo sapiens
gagttctttgcaccctctgc

SEQ ID NO: 598 - DNA, 20 bp, Homo sapiens
gccacaggcagcttactcac

SEQ ID NO: 599 - DNA, 20 bp, Homo sapiens
aggctgcctgccctgcccca

SEQ ID NO: 600 - DNA, 20 bp, Homo sapiens
ccggcccagtgggtcatcag

SEQ ID NO: 601 - DNA, 20 bp, Homo sapiens
ctctcgaggtggaaaggcag

SEQ ID NO: 602 - DNA, 20 bp, Homo sapiens
gattgcctggactcgggcat

SEQ ID NO: 603 - DNA, 20 bp, Homo sapiens
tccttgcttggggctctggg

SEQ ID NO: 604 - DNA, 20 bp, Homo sapiens
gcagagaaggctgcggtggc

SEQ ID NO: 605 - DNA, 20 bp, Homo sapiens
accgtgacctccaaggcgag

SEQ ID NO: 606 - DNA, 20 bp, Homo sapiens
caggacgctgtggatctccg

SEQ ID NO: 607 - DNA, 20 bp, Homo sapiens
aggaagcagccgtgtcccta

SEQ ID NO: 608 - DNA, 20 bp, Homo sapiens
acgcaggaagttgtccaggc

SEQ ID NO: 609 - DNA, 20 bp, Homo sapiens
gaggtccccaagaggaaaac

SEQ ID NO: 610 - DNA, 20 bp, Homo sapiens
cccccagcttcttcccatcc

SEQ ID NO: 611 - DNA, 20 bp, Homo sapiens
attcccacagactcatggtg

SEQ ID NO: 612 - DNA, 20 bp, Homo sapiens
tccaaggcgagcggcaggag

SEQ ID NO: 613 - DNA, 20 bp, Homo sapiens
gctgggagctcagatccata

SEQ ID NO: 614 - DNA, 20 bp, Homo sapiens
tgggggcccaggcatcccca

SEQ ID NO: 615 - DNA, 20 bp, Homo sapiens
gggtgcaaagaactctctgg

SEQ ID NO: 616 - DNA, 20 bp, Homo sapiens
gcgggactagagggagctga

SEQ ID NO: 617 - DNA, 20 bp, Homo sapiens
actggagaagaagaagatcc

SEQ ID NO: 618 - DNA, 20 bp, Homo sapiens
ccagctcttgacccggcctt

SEQ ID NO: 619 - DNA, 20 bp, Homo sapiens
gaactttggggggttcgagg

SEQ ID NO: 620 - DNA, 20 bp, Homo sapiens
gaaaccagttcactgccagc

SEQ ID NO: 621 - DNA, 20 bp, Homo sapiens
acagccgtcagggcacccca

SEQ ID NO: 622 - DNA, 20 bp, Homo sapiens
ccaccccgagccccttacac

SEQ ID NO: 623 - DNA, 20 bp, Homo sapiens
tctcggcagccagctgctgg

SEQ ID NO: 624 - DNA, 20 bp, Homo sapiens
agagagctggactgggatga

SEQ ID NO: 625 - DNA, 20 bp, Homo sapiens
cctttccacctcgagagtac

SEQ ID NO: 626 - DNA, 20 bp, Homo sapiens
aagctggcctacgagtctga

SEQ ID NO: 627 - DNA, 20 bp, Homo sapiens
gtctgtgggagccagtctgt

SEQ ID NO: 628 - DNA, 20 bp, Homo sapiens
agacctatgccccatcaggc

SEQ ID NO: 629 - DNA, 20 bp, Homo sapiens
tgggaagaagctgggggccc

SEQ ID NO: 630 - DNA, 20 bp, Homo sapiens
ctgtggagagaagcctgtcc

SEQ ID NO: 631 - DNA, 20 bp, Homo sapiens
gggacttctaattgctgaga

SEQ ID NO: 632 - DNA, 20 bp, Homo sapiens
ggactcgggcattggctccc

SEQ ID NO: 633 - DNA, 20 bp, Homo sapiens
catctgccacagagcgctgg

SEQ ID NO: 634 - DNA, 20 bp, Homo sapiens
cttctgggagtggaggctcc

SEQ ID NO: 635 - DNA, 20 bp, Homo sapiens
gcccccagtgcagagcccag

SEQ ID NO: 636 - DNA, 20 bp, Homo sapiens
tttgtccttgcttggggctc

SEQ ID NO: 637 - DNA, 20 bp, Homo sapiens
gtggggctggcttccaggac

SEQ ID NO: 638 - DNA, 20 bp, Homo sapiens
tcaagagctggccgctgagg

SEQ ID NO: 639 - DNA, 20 bp, Homo sapiens
cctctagtcccgcggggtgg

SEQ ID NO: 640 - DNA, 20 bp, Homo sapiens
gctcatctgccacagagcgc

SEQ ID NO: 641 - DNA, 20 bp, Homo sapiens
catgagtctgtgggaatttg

SEQ ID NO: 642 - DNA, 20 bp, Homo sapiens
tgcgaggctcctgcctgatg

SEQ ID NO: 643 - DNA, 20 bp, Homo sapiens
ggagtgagtccagcgtctgt

SEQ ID NO: 644 - DNA, 20 bp, Homo sapiens
tgcaaagaactctctggagg

SEQ ID NO: 645 - DNA, 20 bp, Homo sapiens
cacagcgtcctgcagaagct

SEQ ID NO: 646 - DNA, 20 bp, Homo sapiens
cagcttactcactggggtgc

SEQ ID NO: 647 - DNA, 20 bp, Homo sapiens
actgatgtgggtgggggcag

SEQ ID NO: 648 - DNA, 20 bp, Homo sapiens
gcaggatgtgctggtctggg

SEQ ID NO: 649 - DNA, 20 bp, Homo sapiens
tcacagtgtttgtgccatcc

SEQ ID NO: 650 - DNA, 20 bp, Homo sapiens
gtttgtgccatcctggagga

SEQ ID NO: 651 - DNA, 20 bp, Homo sapiens
tcctgaaggactgatgtggg

SEQ ID NO: 652 - DNA, 20 bp, Homo sapiens
tgttagcagagagctggact

SEQ ID NO: 653 - DNA, 20 bp, Homo sapiens
cagtgtttgtgccatcctgg

SEQ ID NO: 654 - DNA, 20 bp, Homo sapiens
agtctgtcagggcctctggg

SEQ ID NO: 655 - DNA, 20 bp, Homo sapiens
tctcgaggtggaaaggcagg

SEQ ID NO: 656 - DNA, 20 bp, Homo sapiens
agactggctcccacagacgc

SEQ ID NO: 657 - DNA, 20 bp, Homo sapiens
agccactcactttggagcac

SEQ ID NO: 658 - DNA, 20 bp, Homo sapiens
tcccaggattgcctggactc

SEQ ID NO: 659 - DNA, 20 bp, Homo sapiens
cctggaactgcagatgaagg

SEQ ID NO: 660 - DNA, 20 bp, Homo sapiens
ggggcgcttcccacagctcc

SEQ ID NO: 661 - DNA, 20 bp, Homo sapiens
cagcccctgggctctgcact

SEQ ID NO: 662 - DNA, 20 bp, Homo sapiens
gcgcgggtgggtagtcggca

SEQ ID NO: 663 - DNA, 20 bp, Homo sapiens
gccccaagcaaggacaaaaa

SEQ ID NO: 664 - DNA, 20 bp, Homo sapiens
agcctggatgggaagaagct

SEQ ID NO: 665 - DNA, 20 bp, Homo sapiens
cagctcttgacccggccttg

SEQ ID NO: 666 - DNA, 20 bp, Homo sapiens
taggggtgccaccaccccgc

SEQ ID NO: 667 - DNA, 20 bp, Homo sapiens
tccactcccagaagaggaaa

SEQ ID NO: 668 - DNA, 20 bp, Homo sapiens
ggaagcgcttcatcgaggag

SEQ ID NO: 669 - DNA, 20 bp, Homo sapiens
gcatcctgctggcagtgaac

SEQ ID NO: 670 - DNA, 20 bp, Homo sapiens
tggatgaatagcccagcttc

SEQ ID NO: 671 - DNA, 20 bp, Homo sapiens
acacgggacagccaccgagc

SEQ ID NO: 672 - DNA, 20 bp, Homo sapiens
gggctcctgaaggactgatg

SEQ ID NO: 673 - DNA, 20 bp, Homo sapiens
cagcctggatgggaagaagc

SEQ ID NO: 674 - DNA, 20 bp, Homo sapiens
ttttcctcttctgggagtgg

SEQ ID NO: 675 - DNA, 20 bp, Homo sapiens
ctccaggttgggagccttag

SEQ ID NO: 676 - DNA, 20 bp, Homo sapiens
gggagctgagggcaggggtc

SEQ ID NO: 677 - DNA, 20 bp, Homo sapiens
agatgaaggtggacttcttc

SEQ ID NO: 678 - DNA, 20 bp, Homo sapiens
tttggccgggccatgggtgc

SEQ ID NO: 679 - DNA, 20 bp, Homo sapiens
ctcgcacctagcgggggcag

SEQ ID NO: 680 - DNA, 20 bp, Homo sapiens
cccgtgtaaggggctcgggg

SEQ ID NO: 681 - DNA, 20 bp, Homo sapiens
tgccggcccagtgggtcatc

SEQ ID NO: 682 - DNA, 20 bp, Homo sapiens
aaaggcagagaaggctgcgg

SEQ ID NO: 683 - DNA, 20 bp, Homo sapiens
aggagcccgtggggcagggc

SEQ ID NO: 684 - DNA, 20 bp, Homo sapiens
taaggggctcggggtgggcc

SEQ ID NO: 685 - DNA, 20 bp, Homo sapiens
acaccatcacgacgcgtggg

SEQ ID NO: 686 - DNA, 20 bp, Homo sapiens
ctggcaggagcccgtggggc

SEQ ID NO: 687 - DNA, 20 bp, Homo sapiens
ccggccttggggtgccctga

SEQ ID NO: 688 - DNA, 20 bp, Homo sapiens
ctgtgttagaccctcttggc

SEQ ID NO: 689 - DNA, 20 bp, Homo sapiens
gtgatgggcacgtcgggccg

SEQ ID NO: 690 - DNA, 20 bp, Homo sapiens
gcccctgggctctgcactgg

SEQ ID NO: 691 - DNA, 20 bp, Homo sapiens
ctgggtgagctggtgaaaca

SEQ ID NO: 692 - DNA, 20 bp, Homo sapiens
ggctgctccttcctccagga

SEQ ID NO: 693 - DNA, 20 bp, Homo sapiens
acagcctccaccaggtgcgg

SEQ ID NO: 694 - DNA, 20 bp, Homo sapiens
tgcccgagtccaggcaatcc

SEQ ID NO: 695 - DNA, 20 bp, Homo sapiens
aggtggaaaggcagggggcg

SEQ ID NO: 696 - DNA, 20 bp, Homo sapiens
cgacagattcattgtgaagc

SEQ ID NO: 697 - DNA, 20 bp, Homo sapiens
gcggggtggtggcaccccta

SEQ ID NO: 698 - DNA, 20 bp, Homo sapiens
ggcaatcctgggagacgtac

SEQ ID NO: 699 - DNA, 20 bp, Homo sapiens
gccgctcgccttggaggtca

SEQ ID NO: 700 - DNA, 20 bp, Homo sapiens
tcactgccagcaggatgccc

SEQ ID NO: 701 - DNA, 20 bp, Homo sapiens
cctgaaggactgatgtgggt

SEQ ID NO: 702 - DNA, 20 bp, Homo sapiens
gtgcgaggctcctgcctgat

SEQ ID NO: 703 - DNA, 20 bp, Homo sapiens
gcacctggtggaggctgtga

SEQ ID NO: 704 - DNA, 20 bp, Homo sapiens
tcacagcctccaccaggtgc

SEQ ID NO: 705 - DNA, 20 bp, Homo sapiens
gccgctctgggtggaagaat

SEQ ID NO: 706 - DNA, 20 bp, Homo sapiens
gactagagggagctgagggc

SEQ ID NO: 707 - DNA, 20 bp, Homo sapiens
tcagctccctctagtcccgc

SEQ ID NO: 708 - DNA, 20 bp, Homo sapiens
ggagcctccactcccagaag

SEQ ID NO: 709 - DNA, 20 bp, Homo sapiens
agaccctcttggcgggaccc

SEQ ID NO: 710 - DNA, 20 bp, Homo sapiens
ccaccttcatctgcagttcc

SEQ ID NO: 711 - DNA, 20 bp, Homo sapiens
gggagtggaggctccaggtt

SEQ ID NO: 712 - DNA, 20 bp, Homo sapiens
cagtgaactggtttctggag

SEQ ID NO: 713 - DNA, 20 bp, Homo sapiens
tcacctgtgatgggcacgtc

SEQ ID NO: 714 - DNA, 20 bp, Homo sapiens
tgccagcaggatgccccggc

SEQ ID NO: 715 - DNA, 20 bp, Homo sapiens
accctcttggcgggaccctg

SEQ ID NO: 716 - DNA, 20 bp, Homo sapiens
tgggggcagcttggccgctc

SEQ ID NO: 717 - DNA, 20 bp, Homo sapiens
aggaggaggccctggtgagc

SEQ ID NO: 718 - DNA, 20 bp, Homo sapiens
ctggaggtgggagccatgca

SEQ ID NO: 719 - DNA, 20 bp, Homo sapiens
tctggaggtgggagccatgc

SEQ ID NO: 720 - DNA, 20 bp, Homo sapiens
cctggatgggaagaagctgg

SEQ ID NO: 721 - DNA, 20 bp, Homo sapiens
ccagcccctgggctctgcac

SEQ ID NO: 722 - DNA, 20 bp, Homo sapiens
ggagtggaagcgcttcatcg

SEQ ID NO: 723 - DNA, 20 bp, Homo sapiens
tgtagcccgtgtaaggggct

SEQ ID NO: 724 - DNA, 20 bp, Homo sapiens
ggagtgaggagggccgggga

SEQ ID NO: 725 - DNA, 20 bp, Homo sapiens
gaggtcacggtatgtgtcgt

SEQ ID NO: 726 - DNA, 20 bp, Homo sapiens
ctagagggagctgagggcag

SEQ ID NO: 727 - DNA, 20 bp, Homo sapiens
tggtgtgtttcccccgcacc

SEQ ID NO: 728 - DNA, 20 bp, Homo sapiens
ctgatgtgggtgggggcagt

SEQ ID NO: 729 - DNA, 20 bp, Homo sapiens
agggccggggagggcaggct

SEQ ID NO: 730 - DNA, 20 bp, Homo sapiens
tgagctatgagtggcccctg

SEQ ID NO: 731 - DNA, 20 bp, Homo sapiens
tcttacgcaggaagttgtcc

SEQ ID NO: 732 - DNA, 20 bp, Homo sapiens
gttccgacatctggctctcc

SEQ ID NO: 733 - DNA, 20 bp, Homo sapiens
agggggcgcgggtgggtagt

SEQ ID NO: 734 - DNA, 20 bp, Homo sapiens
cgctggcctgctccttggcc

SEQ ID NO: 735 - DNA, 20 bp, Homo sapiens
gaaaggcagggggcgcgggt

SEQ ID NO: 736 - DNA, 20 bp, Homo sapiens
tagcccgtgtaaggggctcg

SEQ ID NO: 737 - DNA, 20 bp, Homo sapiens
ctgagggcaggggtccggtg

SEQ ID NO: 738 - DNA, 20 bp, Homo sapiens
acacagcttagtatacacgc

SEQ ID NO: 739 - DNA, 20 bp, Homo sapiens
ccgtcagggcaccccaaggc

SEQ ID NO: 740 - DNA, 20 bp, Homo sapiens
ggcaggggtccggtgaggtc

SEQ ID NO: 741 - DNA, 20 bp, Homo sapiens
ggacttgtaggagaggatct

SEQ ID NO: 742 - DNA, 20 bp, Homo sapiens
tcccagccatgggaacaagg

SEQ ID NO: 743 - DNA, 20 bp, Homo sapiens
gacttctaattgctgagaag

SEQ ID NO: 744 - DNA, 20 bp, Homo sapiens
ggctcctgaaggactgatgt

SEQ ID NO: 745 - DNA, 20 bp, Homo sapiens
tggcaggagcccgtggggca

SEQ ID NO: 746 - DNA, 20 bp, Homo sapiens
agacaggtgagaggaagggc

SEQ ID NO: 747 - DNA, 20 bp, Homo sapiens
tcggaactttggggggttcg

SEQ ID NO: 748 - DNA, 20 bp, Homo sapiens
gcctggatgggaagaagctg

SEQ ID NO: 749 - DNA, 20 bp, Homo sapiens
tgaaggactgatgtgggtgg

SEQ ID NO: 750 - DNA, 20 bp, Homo sapiens
ctgggggcccaggcatcccc

SEQ ID NO: 751 - DNA, 20 bp, Homo sapiens
gagcccccagtgcagagccc

SEQ ID NO: 752 - DNA, 20 bp, Homo sapiens
ggcgcgggtgggtagtcggc

SEQ ID NO: 753 - DNA, 20 bp, Homo sapiens
ccgtgtaaggggctcggggt

SEQ ID NO: 754 - DNA, 20 bp, Homo sapiens
gtcgtgatggtgtgaacacc

SEQ ID NO: 755 - DNA, 20 bp, Homo sapiens
acgacgcgtgggtggcaagc

SEQ ID NO: 756 - DNA, 20 bp, Homo sapiens
gggggcagtggcagcagctt

SEQ ID NO: 757 - DNA, 20 bp, Homo sapiens
agcgtgtatactaagctgtg

SEQ ID NO: 758 - DNA, 20 bp, Homo sapiens
gctcctgcctgatggggcat

SEQ ID NO: 759 - DNA, 20 bp, Homo sapiens
gtctgtcagggcctctggga

SEQ ID NO: 760 - DNA, 20 bp, Homo sapiens
gtagcccgtgtaaggggctc

SEQ ID NO: 761 - DNA, 20 bp, Homo sapiens
agcccctgggctctgcactg

SEQ ID NO: 762 - DNA, 20 bp, Homo sapiens
gtgaactggtttctggagcg

SEQ ID NO: 763 - DNA, 20 bp, Homo sapiens
ctacgagtctgacgggatcg

SEQ ID NO: 764 - DNA, 20 bp, Homo sapiens
agtgaactggtttctggagc

SEQ ID NO: 765 - DNA, 20 bp, Homo sapiens
acgcgtgggtggcaagcggg

SEQ ID NO: 766 - DNA, 20 bp, Homo sapiens
cgcgggactagagggagctg

SEQ ID NO: 767 - DNA, 20 bp, Homo sapiens
ggcaggagtgaggagggccg

SEQ ID NO: 768 - DNA, 20 bp, Homo sapiens
aagtgagtggcttcttacgc

SEQ ID NO: 769 - DNA, 20 bp, Homo sapiens
ctgaaggactgatgtgggtg

SEQ ID NO: 770 - DNA, 20 bp, Homo sapiens
ttgccacccacgcgtcgtga

SEQ ID NO: 771 - DNA, 20 bp, Homo sapiens
agggcaggagtgaggagggc

SEQ ID NO: 772 - DNA, 20 bp, Homo sapiens
tcttcttctccagttcccgc

SEQ ID NO: 773 - DNA, 20 bp, Homo sapiens
aggagtgaggagggccgggg

SEQ ID NO: 774 - DNA, 20 bp, Homo sapiens
actcccagaagaggaaaagg

SEQ ID NO: 775 - DNA, 20 bp, Homo sapiens
tgaggagggccggggagggc

SEQ ID NO: 776 - DNA, 20 bp, Homo sapiens
acctggtggaggctgtgatg

SEQ ID NO: 777 - DNA, 20 bp, Homo sapiens
cagggccgaggcctcctcag

SEQ ID NO: 778 - DNA, 20 bp, Homo sapiens
tactctcgaggtggaaaggc

SEQ ID NO: 779 - DNA, 20 bp, Homo sapiens
ttggggctctggggggtgag

SEQ ID NO: 780 - DNA, 20 bp, Homo sapiens
gctcctggacccccagcagc

SEQ ID NO: 781 - DNA, 20 bp, Homo sapiens
ggggggtgagaggagagcat

SEQ ID NO: 782 - DNA, 20 bp, Homo sapiens
cggcccagtgggtcatcagg

SEQ ID NO: 783 - DNA, 20 bp, Homo sapiens
aggaagggcaggagtgagga

SEQ ID NO: 784 - DNA, 20 bp, Homo sapiens
gagggccggggagggcaggc

SEQ ID NO: 785 - DNA, 20 bp, Homo sapiens
tgctgggggtccaggagctg

SEQ ID NO: 786 - DNA, 20 bp, Homo sapiens
gctgggggtccaggagctgt

SEQ ID NO: 787 - DNA, 20 bp, Homo sapiens
tgagaggaagggcaggagtg

SEQ ID NO: 788 - DNA, 20 bp, Homo sapiens
tgggagtggaggctccaggt

SEQ ID NO: 789 - DNA, 20 bp, Homo sapiens
gaggaagggcaggagtgagg

SEQ ID NO: 790 - DNA, 20 bp, Mus musculus
gctggctgtgaactggtttc

SEQ ID NO: 791 - DNA, 20 bp, Mus musculus
ctagttcccgaaggatgtgc

SEQ ID NO: 792 - DNA, 20 bp, Mus musculus
attggagaccaccactccgt

SEQ ID NO: 793 - DNA, 20 bp, Mus musculus
ttccctcctctgccagccat

SEQ ID NO: 794 - DNA, 20 bp, Mus musculus
cgaaggaagttgtccaggct

SEQ ID NO: 795 - DNA, 20 bp, Mus musculus
atacctgtgataggcacatc

SEQ ID NO: 796 - DNA, 20 bp, Mus musculus
gacttccttgttcccatggc

SEQ ID NO: 797 - DNA, 20 bp, Mus musculus
ggccttcgaatccgacggag

SEQ ID NO: 798 - DNA, 20 bp, Homo sapiens
ccttatgaaaaagtcaaaac

SEQ ID NO: 799 - DNA, 20 bp, Homo sapiens
aaaatatcaagtatatatgg

SEQ ID NO: 800 - DNA, 20 bp, Homo sapiens
tctagcatcggcatgccaaa

SEQ ID NO: 801 - DNA, 20 bp, Homo sapiens
ttggaagctcatggacaaag

SEQ ID NO: 802 - DNA, 20 bp, Homo sapiens
gatttcctcctcgaccacca

SEQ ID NO: 803 - DNA, 20 bp, Homo sapiens
cttcatctcttggatcaaag

SEQ ID NO: 804 - DNA, 20 bp, Homo sapiens
aatgtatgaagaacagtcac

SEQ ID NO: 805 - DNA, 20 bp, Homo sapiens
taaacttacctgaaacagcc

SEQ ID NO: 806 - DNA, 20 bp, Homo sapiens
aagaatatgatgttcctccc

SEQ ID NO: 807 - DNA, 20 bp, Homo sapiens
agcaagctgccgcagatcgc

SEQ ID NO: 808 - DNA, 20 bp, Homo sapiens
agtactcattctcactgagt

SEQ ID NO: 809 - DNA, 20 bp, Mus musculus
tctttgttgcaggagtctga

SEQ ID NO: 810 - DNA, 20 bp, Mus musculus
caggggcttgttatgaggta

SEQ ID NO: 811 - DNA, 20 bp, Mus musculus
ctgattgatggtagcaggga

SEQ ID NO: 812 - DNA, 20 bp, Mus musculus
ccttatcttcagtcacatgc

SEQ ID NO: 813 - DNA, 20 bp, Mus musculus
tcacatgctggcagaaatca

SEQ ID NO: 814 - DNA, 20 bp, Mus musculus
ttctgtcgctgtgagataaa

SEQ ID NO: 815 - DNA, 20 bp, Mus musculus
acaaggcagtacctgccacg

SEQ ID NO: 816 - DNA, 20 bp, Mus musculus
tgtgactcacccgggataca

SEQ ID NO: 817 - DNA, 20 bp, Mus musculus
gaggtccatcagatcagctc

SEQ ID NO: 818 - DNA, 20 bp, Mus musculus
atctccctggaactggccat

SEQ ID NO: 819 - DNA, 20 bp, Mus musculus
tgcaaaaattgcaaaactca

SEQ ID NO: 820 - DNA, 20 bp, Mus musculus
tgcacagaactattgtacca

SEQ ID NO: 821 - DNA, 20 bp, Mus musculus
cagattagtgcttaccttcc

SEQ ID NO: 822 - DNA, 20 bp, Mus musculus
attccgtaaaatagagcccc

SEQ ID NO: 823 - DNA, 20 bp, Mus musculus
ctgcactcggctgggacaat

SEQ ID NO: 824 - DNA, 20 bp, Homo sapiens
agtccatatggaacccacgg

SEQ ID NO: 825 - DNA, 20 bp, Homo sapiens
agtctgagtgcaaattgggc

SEQ ID NO: 826 - DNA, 20 bp, Homo sapiens
tacgaattgcaccggaccag

SEQ ID NO: 827 - DNA, 20 bp, Homo sapiens
ttagaggcttgaggaaaccg

SEQ ID NO: 828 - DNA, 20 bp, Homo sapiens
ttagaacctatgaagctctg

SEQ ID NO: 829 - DNA, 20 bp, Homo sapiens
cctgaataaactccaccgca

SEQ ID NO: 830 - DNA, 20 bp, Homo sapiens
aattcgaaagcccatcagtt

SEQ ID NO: 831 - DNA, 20 bp, Homo sapiens
tggccacaacccaaactgat

SEQ ID NO: 832 - DNA, 20 bp, Homo sapiens
cagcatactctgaggtacga

SEQ ID NO: 833 - DNA, 20 bp, Homo sapiens
ttacctctagcactgctgag

SEQ ID NO: 834 - DNA, 20 bp, Homo sapiens
tatgcagtccattattgaca

SEQ ID NO: 835 - DNA, 20 bp, Homo sapiens
gtaacacagcttattccgcg

SEQ ID NO: 836 - DNA, 20 bp, Homo sapiens
actttccctagcaatgcagg

SEQ ID NO: 837 - DNA, 20 bp, Mus musculus
caaatgggcaagccttacgg

SEQ ID NO: 838 - DNA, 20 bp, Mus musculus
ctcaatgtccgtattgatag

SEQ ID NO: 839 - DNA, 20 bp, Mus musculus
agtctgagtgcaaattgggc

SEQ ID NO: 840 - DNA, 20 bp, Mus musculus
ccagatagtgcaaattgcta

SEQ ID NO: 841 - DNA, 20 bp, Mus musculus
tgatagtggtctggtcaaat

SEQ ID NO: 842 - DNA, 20 bp, Mus musculus
aattcgaaagcccatcagtt

SEQ ID NO: 843 - DNA, 20 bp, Mus musculus
gcccattactttgtgtagtg

SEQ ID NO: 844 - DNA, 20 bp, Mus musculus
tggccacagcccaaactgat

SEQ ID NO: 845 - DNA, 20 bp, Homo sapiens
cgtccgcgccatgttccagg

SEQ ID NO: 846 - DNA, 20 bp, Homo sapiens
tggtttcaggagccctgtaa

SEQ ID NO: 847 - DNA, 20 bp, Homo sapiens
acccggatacagcagcagct

SEQ ID NO: 848 - DNA, 20 bp, Homo sapiens
ttccagggctccgagccgcg

SEQ ID NO: 849 - DNA, 20 bp, Homo sapiens
ctgaaggctaccaactacaa

SEQ ID NO: 850 - DNA, 20 bp, Homo sapiens
gggtatttcctcgaaagtct

SEQ ID NO: 851 - DNA, 20 bp, Homo sapiens
gagccgcaggaggtgccgcg

SEQ ID NO: 852 - DNA, 20 bp, Homo sapiens
ctgagtcaggactcccacgc

SEQ ID NO: 853 - DNA, 20 bp, Homo sapiens
cacttacgagtccccgtcct

SEQ ID NO: 854 - DNA, 20 bp, Homo sapiens
ctcaaattccttttggtttc

SEQ ID NO: 855 - DNA, 20 bp, Homo sapiens
ggttggtgatcacagccaag

SEQ ID NO: 856 - DNA, 20 bp, Homo sapiens
gcaggttgttctggaagttg

SEQ ID NO: 857 - DNA, 20 bp, Mus musculus
cctcgaaagtctcggagctc

SEQ ID NO: 858 - DNA, 20 bp, Mus musculus
ctgcgtcaagactgctacac

SEQ ID NO: 859 - DNA, 20 bp, Mus musculus
tgctcacaggcaagatgtag

SEQ ID NO: 860 - DNA, 20 bp, Mus musculus
ccggacagccctccaccttg

SEQ ID NO: 861 - DNA, 20 bp, Mus musculus
agacctaccattgtagttgg

SEQ ID NO: 862 - DNA, 20 bp, Mus musculus
ccaagtgctccacgatggcc

SEQ ID NO: 863 - DNA, 20 bp, Mus musculus
agcctctatccacggctacc

SEQ ID NO: 864 - DNA, 20 bp, Mus musculus
gccccaggtaagctggtagg

SEQ ID NO: 865 - DNA, 20 bp, Mus musculus
gcaagcagcgcacctgctgc

SEQ ID NO: 866 - DNA, 20 bp, Mus musculus
tcaagactgctacactggcc

SEQ ID NO: 867 - DNA, 20 bp, Mus musculus
gcaggttgttctggaagttg

SEQ ID NO: 868 - DNA, 20 bp, Mus musculus
gggtgctgatgtcaacgctc

SEQ ID NO: 869 - DNA, 20 bp, Mus musculus
ccacgatggccaggtagccg

SEQ ID NO: 870 - DNA, 20 bp, Mus musculus
tggtcagcggcttctcttcg

SEQ ID NO: 871 - DNA, 20 bp, Mus musculus
aatgtggggctgatgtcaac

SEQ ID NO: 872 - DNA, 20 bp, Mus musculus
atttcaacaagagcgaaacc

SEQ ID NO: 873 - DNA, 20 bp, Mus musculus
cacctgaccaatgacttcca

SEQ ID NO: 874 - DNA, 20 bp, Mus musculus
gccctggaagcagcagctca

SEQ ID NO: 875 - DNA, 20 bp, Mus musculus
gctcacaggcaagatgtaga

SEQ ID NO: 876 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 877 - protein, 220 aa, Homo sapiens
MLRLLLALNLFPSIQVTGNKILVKQSPMLVAYDNAVNLSCKYSYNLFSREFRASLHKGLDSAVEVCVVYGNYSQQLQVYSKTGFNCDGKLGNESVTFYLQNLYVNQTDIYFCKIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 878 - protein, 351 aa, Homo sapiens
MSFPCKFVASFLLIFNVSSKGAVSKEITNALETWGALGQDINLDIPSFQMSDDIDDIKWEKTSDKKKIAQFRKEKETFKEKDTYKLFKNGTLKIKHLKTDDQDIYKVSIYDTKGKNVLEKIFDLKIQERVSKPKISWTCINTTLTCEVMNGTDPELNLYQDGKHLKLSQRVITHKWTTSLSAKFKCTAGNKVSKESSVEPVSCPEKGLDIYLIIGICGGGSLLMVFVALLVFYITKRKKQRSRRNDEELETRAHRVATEERGRKPHQIPASTPQNPATSQHPPPPPGHRSQAPSHRPPPPGHRVQHQPQKRPPAPSGTQVHQQKGPPLPRPRVQPKPPHGAAENSLSPSSN

SEQ ID NO: 879 - protein, 153 aa, Homo sapiens
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 880 - protein, 255 aa, Homo sapiens
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 881 - protein, 254 aa, Homo sapiens
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 882 - protein, 142 aa, Homo sapiens
PNIQNPDPAVYQLRDSKSSDKSVCLFTDFDSQTNVSQSKDSDVYITDKTVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 883 - protein, 178 aa, Homo sapiens
DLKNVFPPEVAVFEPSEAEISHTQKATLVCLATGFYPDHVELSWWVNGKEVHSGVSTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 884 - protein, 9 aa, Artificial Sequence
  Synthetic
SLLMWITQC

SEQ ID NO: 885 - protein, 274 aa, Homo sapiens
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 886 - protein, 311 aa, Homo sapiens
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 887 - protein, 274 aa, Artificial Sequence
  Synthetic, 95LY_NyESO_TCRa
METLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 888 - protein, 311 aa, Artificial Sequence; identical to SEQ ID NO: 886
  Synthetic, 95LY_NyESO_TCRb

SEQ ID NO: 889 - protein, 9 aa, Artificial Sequence
  Synthetic
AAGIGILTV

SEQ ID NO: 890 - protein, 268 aa, Artificial Sequence
  Synthetic, DMF4_MART_TCRa
MLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 891 - protein, 311 aa, Artificial Sequence
  Synthetic, DMF4_MART_TCRb
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 892 - protein, 272 aa, Artificial Sequence
  Synthetic, DMF5_MART_TCRa
MKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 893 - protein, 310 aa, Artificial Sequence
  Synthetic, DMF5_MART_TCRb
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 894 - protein, 9 aa, Artificial Sequence
  Synthetic
RMFPNAPYL

SEQ ID NO: 895 - protein, 270 aa, Artificial Sequence
  Synthetic, DLT_WT1_TCRa
MTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATEDLTLIWGAGTKLIIKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 896 - protein, 310 aa, Artificial Sequence
  Synthetic, DLT_WT1_TCRb
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGALYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRG

SEQ ID NO: 897 - protein, 22 aa, Artificial Sequence
  Synthetic, P2A_linker
GSGATNFSLLKQAGDVEENPGP

SEQ ID NO: 898 - protein, 607 aa, Artificial sequence
  Synthetic, IG4_NyESO_TCRa_TCR
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPTSGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 899 - protein, 607 aa, Artificial Sequence
  Synthetic, 95LY_NyESO_TCRa_TCRb
MSIGLLCCAALSLLWAGPVNAGVTQTPKFQVLKTGQSMTLQCAQDMNHEYMSWYRQDPGMGLRLIHYSVGAGITDQGEVPNGYNVSRSTTEDFPLRLLSAAPSQTSVYFCASSYVGNTGELFFGEGSRLTVLEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMETLLGLLILWLQLQWVSSKQEVTQIPAALSVPEGENLVLNCSFTDSAIYNLQWFRQDPGKGLTSLLLIQSSQREQTSGRLNASLDKSSGRSTLYIAASQPGDSATYLCAVRPLYGGSYIPTFGRGTSLIVHPYIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 900 - protein, 601 aa, Artificial sequence
  Synthetic, DMF4_MART_TCRa_TCRb
MGTRLFFYVALCLLWTGHMDAGITQSPRHKVTETGTPVTLRCHQTENHRYMYWYRQDPGHGLRLIHYSYGVKDTDKGEVSDGYSVSRSKTEDFLLTLESATSSQTSVYFCAISEVGVGQPQHFGDGTRLSILEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMLLEHLLIILWMQLTWVSGQQLNQSPQSMFIQEGEDVSMNCTSSSIFNTWLWYKQEPGEGPVLLIALYKAGELTSNGRLTAQFGITRKDSFLNISASIPSDVGIYFCAGGTGNQFYFGTGTSLTVIPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 901 - protein, 604 aa, Artificial sequence
  Synthetic, DMF5_MART_TCRa_TCRb
MRIRLLCCVAFSLLWAGPVIAGITQAPTSQILAAGRRMTLRCTQDMRHNAMYWYRQDLGLGLRLIHYSNTAGTTGKGEVPDGYSVSRANTDDFPLTLASAVPSQTSVYFCASSLSFGTEAFFGQGTRLTVVEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMKSLRVLLVILWLQLSWVWSQQKEVEQNSGPLSVPEGAIASLNCTYSDRGSQSFFWYRQYSGKSPELIMFIYSNGDKEDGRFTAQLNKASQYVSLLIRDSQPSDSATYLCAVNFGGGKLIFGQGTELSVKPNIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 902 - protein, 602 aa, Artificial Sequence
  Synthetic, DLT_WT1_TCRa_TCRb
MSNQVLCCVVLCFLGANTVDGGITQSPKYLFRKEGQNVTLSCEQNLNHDAMYWYRQDPGQGLRLIYYSQIVNDFQKGDIAEGYSVSREKKESFPLTVTSAQKNPTAFYLCASSPGALYEQYFGPGTRLTVTEDLKNVFPPEVAVFEPSEAEISHTQKATLVCLATGFYPDHVELSWWVNGKEVHSGVCTDPQPLKEQPALNDSRYCLSSRLRVSATFWQNPRNHFRCQVQFYGLSENDEWTQDRAKPVTQIVSAEAWGRADCGFTSESYQQGVLSATILYEILLGKATLYAVLVSALVLMAMVKRKDSRGGSGATNFSLLKQAGDVEENPGPMTSIRAVFIFLWLQLDLVNGENVEQHPSTLSVQEGDSAVIKCTYSDSASNYFPWYKQELGKRPQLIIDIRSNVGEKKDQRIAVTLNKTAKHFSLHITETQPEDSAVYFCAATEDLTLIWGAGTKLIIKPDIQNPDPAVYQLRDSKSSDKSVCLFTDFDSQTNVSQSKDSDVYITDKCVLDMRSMDFKSNSAVAWSNKSDFACANAFNNSIIPEDTFFPSPESSCDVKLVEKSFETDTNLNFQNLSVIGFRILLLKVAGFNLLMTLRLWSS

SEQ ID NO: 903 - protein, 444 aa, Artificial Sequence
  Synthetic
MLLLVTSLLLCELPHPAFLLIPDILLTQSPVILSVSPGERVSFSCRASQSIGTNIHWYQQRTNGSPRLLIKYASESISGIPSRFSGSGSGTDFTLSINSVESEDIADYYCQQNNNWPTTFGAGTKLELKGGGGSGGGGSGGGGSQVQLKQSGPGLVQPSQSLSITCTVSGFSLTNYGVHWVRQSPGKGLEWLGVIWSGGNTDYNTPFTSRLSINKDNSKSQVFFKMNSLQSNDTAIYYCARALTYYDYEFAYWGQGTLVTVSATTTPAPRPPTPAPTIASQPLSLRPEACRPAAGGAVHTRGLDFACDIYIWAPLAGTCGVLLLSLVITLYCRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 904 - DNA, 1328 bp, Artificial Sequence
  Synthetic
atgctcctcctggttactagcttgcttttgtgcgaactgccgcatcctgccttccttctcatcccagatatacttctgacacaatctccggtaatactttccgtctcaccgggggagcgagtgtcattttcatgccgggcgagtcaatccatcgggacgaatattcactggtatcagcaaaggactaacggctcaccacgccttctcatcaagtatgccagtgagtccataagtggcattccctctagattctcaggatcaggcagtggcacggacttcacattgtcaattaatagcgtagaaagtgaggacatagcagattattattgccaacaaaacaataactggcctaccacattcggtgcaggcaccaagcttgagttgaaggggggtggtggttctggaggcggtgggagcggtggtggtgggtcacaagtgcagcttaagcaaagcggaccaggtctggtccaacctagccagtcactgtcaatcacatgtacggtatccggctttagtctgacaaattatggcgtccactgggtaaggcaatcccctggaaagggcctcgagtggttgggggtgatttggagcggaggaaacaccgactataatacccctttcacctccagactgtccataaacaaggacaactctaaaagtcaggtattcttcaaaatgaacagtctgcaaagtaatgacacagcgatatattattgcgcgagagcccttacatactacgattacgagttcgcttattggggacaaggaacgttggtgacggtgtctgccacaaccactcctgctcccaggccaccaacaccggcgcctaccatagcgtcacagccgcttagtctcaggccggaagcgtgtcgccccgcagccggtggggcggtccacacacgcgggctggatttcgcatgcgatatatacatctgggcaccccttgccgggacctgcggtgttttgctcttgtctctcgtaatcacgctgtactgtcgggttaagttttcaagatctgcggatgccccggcataccaacaagggcagaatcagttgtacaacgaactgaacttgggcagacgcgaggagtatgatgtcttggacaagaggcgggggcgcgacccggaaatgggtggcaaaccacggcgcaagaacccacaagaggggctttacaacgaattgcagaaagacaagatggccgaggcatacagcgagattggcatgaaaggagagaggaggaggggaaaggggcatgatggcctttatcagggcctttctactgccaccaaggacacatacgacgcactgcacatgcaggcattgccacc

SEQ ID NO: 905 - protein, 446 aa, Artificial Sequence
  Synthetic
MLLLVTSLLLCELPHPAFLLIPDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGGGGSGGGGSGGGGSEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSTTTPAPRPPTPAPTIASQPLSLRPEACRPAAGGAVHTRGLDFACDIYIWAPLAGTCGVLLLSLVITLYCLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 906 - DNA, 1338 bp, Artificial Sequence
  Synthetic
atgctccttcttgtgacgtcactcctgctttgtgagctgccgcacccggcctttctgctcatacccgacatacaaatgacacagacgacaagttccctttccgcctccttgggcgaccgagtgacaatcagttgccgagcttcccaggacatatctaaatatttgaattggtatcagcaaaagccagatggtacggttaaacttcttatctaccacacctccaggctccattctggggttccgagccgattctctggatctggctcagggaccgattattctttgactatttccaatttggagcaggaagacatcgcaacctatttctgccaacaaggaaatacgctgccatacaccttcggcgggggcaccaaactcgagattactgggggtggggggagtggaggagggggttccggtggaggtgggtcagaagtcaagctgcaagagagtgggcccgggttggttgctcctagccaatccttgagtgtaacatgcaccgttagcggagtttcacttcctgactacggtgttagctggataagacagcccccgaggaagggtctggaatggctgggggtcatttggggcagtgagacgacatattacaacagtgccttgaaatccaggcttacgatcataaaagacaatagtaaaagccaagtgttcctcaagatgaactctcttcagaccgacgacacagccatctactattgcgcaaaacattattattatggaggtagttacgctatggactattggggccaggggacttcagtgacggtgagtagtaccacgactccggcaccgagaccaccaacaccagccccaacaattgcctcacagcccttgagccttagacccgaggcctgtaggcccgccgcaggaggggcagttcatacgcgaggattggactttgcatgtgacatctatatctgggcgccacttgcgggaacttgcggtgtccttttgctctcattggtcattaccctctattgtttgagagtaaaattttcccgctccgctgatgcgcctgcataccagcaaggtcagaaccaactctacaatgagcttaacctcggtagaagagaggaatatgacgtcttggataagaggagaggccgagacccagaaatggggggaaagccgcgccgcaagaatccacaagaaggtctttacaatgaactgcagaaggacaaaatggccgaagcgtatagcgagataggaatgaaaggcgaacggagacggggcaaggggcatgacgggctttaccaaggacttagcacagcgacgaaggatacatacgacgcactgcatatgcaagcgctgccaccgcgc

SEQ ID NO: 907 - protein, 496 aa, Artificial Sequence
  Synthetic
MDFQVQIFSFLLISASVIMSRGATGGATTTTCAGGTGCAGATTTTCAGCTTCCTGCTAATCAGTGCCTCADIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTGSTSGSGKPGSGEGSEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDVWGQGTLVTVSSTTTPAPRPPTPAPTIASQPLSLRPEACRPAAGGAVHTRGLDFACDIYIWAPLAGTCGVLLLSLVITLYCLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 908 - DNA, 1488 bp, Artificial Sequence
  Synthetic
atggactttcaggtgcagatcttctcattcctccttatcagtgcgagtgtaattatgtcaagaggtgctacaggcggtgctaccacgacgacatgcgcagggggtaccggttgtgcaggagcgaccacgactacctgcgcaggttgtaccacctgttgtactggatgtactgctgcgacatgtgcggggacgggttgttgcacttgtgcggacatacaaatgacgcaaagcccgtccagcctgtctgcatcagttggggatagagtgacgattacatgtagagcaagccaggatgttaacaccgccgtagcgtggtaccaacaaaaaccaggtaaagccccgaagctgctcatatactccgccagctttctgtattcaggcgttcccagtcggttcagcggcagcagatcagggacggattttacgctcactatctcttcccttcagcctgaagattttgctacctattattgtcagcagcattacacgactcccccaacttttggtcaggggactaaagttgaaatcaaacgaacgggctccacctcaggtagcggtaagccaggcagcggagaagggtctgaagtccagttggttgagagtggaggcggtcttgtgcaacccggtggcagcttgcgactgagctgtgcagcgtctggcttcaacataaaagatacttatattcattgggtaagacaggctcctggtaaagggctggaatgggtggcacgaatatatccgactaacggttataccagatacgccgattctgttaagggcaggttcacaataagcgccgacacaagtaagaatacggcgtatctgcagatgaattcacttcgagctgaagacacagcggtatactactgctccaggtggggtggggatggtttttatgcgatggacgtttggggtcaaggaacactggtaactgttagttctaccacgacacctgctcctaggccccccacaccggcacctacgatcgcttcccagccgcttagcctccgcccggaggcatgccggcccgctgcggggggagcggtacatactcgcgggttggacttcgcttgcgacatctacatttgggcaccactggcaggcacatgtggcgttctgttgcttagtctggttattacactgtattgcctgcgagttaaattctcccgcagcgctgatgcgcccgcctatcagcaaggtcaaaaccagctgtataatgagcttaatttgggacgccgagaagagtatgacgtccttgacaagaggcgcgggcgcgatccggagatgggtggtaaaccgcgccggaaaaacccccaggaaggcctttacaatgagctccaaaaagataaaatggcagaggcatactctgaaataggaatgaagggcgagagacgccggggtaagggacacgatggcctttatcaagggcttagtacagccacgaaggatacgtatgacgctctgcacatgcaggctcttcccccgaga

SEQ ID NO: 909 - DNA, 20 bp, Artificial Sequence
  Synthetic
gagtttaagccacaaataca

SEQ ID NO: 910 - DNA, 20 bp, Homo sapiens
agatgatgtcaaccaaggag

(19) United States
(12) Patent Application Publication
(10) Pub. No.: US 2024/0376428 A1
(43) Pub. Date: Nov. 14, 2024

(54) METHODS FOR ACTIVATION AND EXPANSION OF TUMOR INFILTRATING LYMPHOCYTES

(71) Applicant: KSQ Therapeutics, Inc., Cambridge, MA (US)

(72) Inventors: Micah Benson, Cambridge, MA (US); Nicholas John Colletti, Cambridge, MA (US); Noah Jacob Tubo, Cambridge, MA (US); Michael R. Schlabach, Cambridge, MA (US)

(73) Assignee: KSQ Therapeutics, Inc., Cambridge, MA (US)

(21) Appl. No.: 17/779,458

(22) PCT Filed: Nov. 24, 2020
(86) PCT No.: PCT/US2020/062094

§ 371 Date: Nov. 22, 2022

Related U.S. Application Data
(60) Provisional application No. 63/081,539, filed on Sep. 22, 2020.
(60) Provisional application No. 62/940,035, filed on Nov. 25, 2019.

(51) Int. Cl. C12N 5/0783; C12N 5/00

(52) U.S. Cl. CPC C12N 5/0636; C12N 5/0068; C12N 2501/2302; C12N 2501/51; C12N 2501/515; C12N 2501/599; C12N 2502/11; C12N 2510/00

(57) ABSTRACT

Methods for activating and expanding tumor infiltrating lymphocytes (TILs) in a one-step process are provided. Methods for activating and expanding TILs without the use of feeder cells are also provided. Compositions of expanded populations of TILs are also provided, in addition to isolated populations of expanded TILs enriched in central memory T cell phenotype.

### RELATED APPLICATIONS

This application application is a national stage filing under 35 U.S.C. § 371 of International Application No. PCT/US2020/062094, filed Nov. 24, 2020, which was published under PCT Article 21(2) in English and claims the benefit under 35 U.S.C. § 119(e) of U.S. Provisional Patent Application No. 62/940,035, filed on Nov. 25, 2019 and U.S. Provisional Patent Application No. 63/081,539, filed on Sep. 22, 2020, each of which is incorporated by reference herein in its entirety.

### REFERENCE TO A SEQUENCE LISTING SUBMITTED AS A TEXT FILE VIA EFS-WEB

The instant application contains a Sequence Listing that has been submitted in ASCII format via EFS-Web and is hereby incorporated by reference in its entirety. Said ASCII copy, created on Aug. 1, 2022, is named K0713.70004US02-SUBSEQ-HJD and is 483,466 bytes in size.

### FIELD OF THE INVENTION

This disclosure relates to methods for activation and expansion of lymphocyte populations, especially of tumor infiltrating lymphocytes.

### BACKGROUND

The adoptive transfer of tumor infiltrating lymphocytes (TILs) is a powerful approach to the treatment of bulky, refractory cancers, especially in patients with poor prognoses. A large number of TILs are required for successful immunotherapy, necessitating a robust and reliable process for expansion. A multi-step process, typically including an IL-2-based TIL expansion “pre-REP” followed by a “rapid expansion protocol” (REP), has become a preferred method for TIL expansion because of its ability to generate a therapeutically effective number of TILs but remains limited due to the time consuming nature of the process. After a pre-REP step that can last for up to 6 weeks, REP can then result in a 1,000-fold expansion of TILs over a 14-day period. REP is a difficult process requiring a large excess (e.g., 200-fold) of feeder cells to activate the TILs, in addition to requiring large doses of anti-CD3 antibody (OKT3) and IL-2.

One of the most challenging aspects of currently existing REP-based TIL expansion methods is the necessity of obtaining and using feeder cells. In these REP-based methods, the activation of TILs depends on the presence of feeder cells. Populations of feeder cells are usually collected from 3-5 allogeneic donors, which makes controlling the process of collecting, irradiating, and maintaining the populations of feeder cells expensive and difficult. Therefore, even though using feeder cells is costly and challenging, they have been assumed to be indispensable in the TIL activation and expansion process.

There is thus a need for TIL expansion methods that are more streamlined.

### SUMMARY

Methods for activating and expanding TILs using more streamlined approaches, including one-step approaches, approaches requiring shorter expansion periods, approaches using soluble reagents for stimulation, approaches more suitable for clinical manufacturing, and approaches without the use of feeder cells, are provided. Compositions of expanded populations of TILs are also provided, in addition to isolated populations of expanded TILs enriched in central memory T cell phenotype.

As disclosed herein, it was found, unexpectedly, that TILs can be activated and expanded using a combination of a T cell receptor (TCR) agonist (e.g., an CD3 agonist) and a CD28 agonist in the absence of feeder cells. The TCR agonist and CD28 agonist can be antibodies linked to or complexed with each other, or linked to nanomatrices. Surprisingly, the feeder cell-free TIL activation and expansion process described herein can result in a 150,000-fold expansion of TILs and also in an enriched central memory T cell phenotype. Surprisingly, the feeder cell-free TIL activation and expansion process described herein can also result in a 4,000 to 100,000-fold expansion by day 14 of the one-step process. This robust expansion was even observed in samples from multiple donors which failed to expand under pre-REP conditions. Thus, the processes described herein are capable of generating expanded TILs in situations where the current standard of practice two-step TIL expansion method fails.

As disclosed herein, it was also found, unexpectedly, that TILs can be activated and expanded using a one-step process that obviates the need for separate pre-REP and REP steps.

In one aspect, the present invention is directed to a method of expanding a population of TILs in a disaggregated tumor sample, the method comprising culturing the disaggregated tumor sample in a medium, wherein the TILs are contacted with a TCR agonist, a CD28 agonist, and a T cell-stimulating cytokine.

In some embodiments, the medium is supplemented with the T cell-stimulating cytokine at a time interval selected from the group consisting of 1 day, 2 days, 3 days, 4 days, 5 days, and 6 days.

In some embodiments, the final concentration of the T cell-stimulating cytokine is 10 U/ml to 7,000 U/ml. In some embodiments, the T cell-stimulating cytokine is IL-2. In some embodiments, the medium is changed at a time interval selected from the group consisting of 1 day, 2 days, 3 days, 4 days, 5 days, and 6 days.

In some embodiments, the components of the medium are maintained. In some embodiments, 30% to 99% of the medium is changed at a time interval selected from the group consisting of 1 day, 2 days, 3 days, 4 days, 5 days, and 6 days.

In some embodiments, the methods herein can rescue TIL samples from a previously failed pre-REP expansion. In some embodiments, the tumor sample is from a subject who had previously submitted a tumor sample for TIL expansion, wherein the previous TIL expansion comprises a pre-REP step and wherein the number of TILs isolated from the pre-REP step was less than 1000 TILs. In some embodiments, the tumor sample is from a subject who had previously submitted a tumor sample for TIL expansion, wherein the previous TIL expansion comprises a pre-REP step and wherein the fold expansion of TILs isolated from the pre-REP step was less than 5 fold.

In some embodiments, the disaggregated tumor sample comprises tumor fragments that are 0.5 to 4 mm³ in size. In some embodiments, the disaggregated tumor sample comprises digested tumor fragments.

In some embodiments, the medium comprises feeder cells. In some embodiments, the feeder cells are peripheral blood mononuclear cells or antigen presenting cells. In some embodiments, the feeder cells express the TCR agonist, the CD28 agonist and/or a 4-1BB agonist. In some embodiments, the TCR agonist, CD28 agonist and/or the 4-1BB agonist are expressed on the surface of the feeder cells. In some embodiments, the feeder cells are genetically modified to express the TCR agonist, the CD28 agonist and/or the 4-1BB ligand. In some embodiments, the TCR agonist is a CD3 agonist. In some embodiments, the CD3 agonist is OKT3. In some embodiments, the CD28 agonist is CD86. In some embodiments, the feeder cells are antigen presenting cells. In some embodiments, the antigen presenting cells comprise K562 cells. In some embodiments, the feeder cells express the TCR agonist and/or a 4-1BB agonist. In some embodiments, the 4-1BB agonist is 4-1BB ligand. In some embodiments, the feeder cells are genetically modified to express the T cell-stimulating cytokine. In some embodiments, the T cell-stimulating cytokine is IL-2.

In some embodiments, the medium does not comprise feeder cells. In some embodiments, the CD28 agonist is soluble in the medium.

In some embodiments, the TCR agonist is a CD3 agonist.

In some embodiments, the TCR agonist and/or the CD28 agonist are linked to a nanomatrix comprising a colloidal suspension of matrices of polymer chains, wherein each nanomatrix is 1 to 500 nm in length in its largest dimension. In some embodiments, the TCR agonist and the CD28 agonist are attached to the same polymer chains. In some embodiments, the TCR agonist and the CD28 agonist are attached to different polymer chains. In some embodiments, the TCR agonist is attached to the nanomatrix at 25 μg per mg of nanomatrix.

In some embodiments, the TCR agonist comprises a soluble monospecific complex comprising two anti-CD3 antibodies linked together. In some embodiments, the CD28 agonist comprises a soluble monospecific complex comprising two anti-CD28 antibodies linked together. In some embodiments, the medium comprises a CD2 agonist. In some embodiments, the CD2 agonist comprises a soluble monospecific complex comprising two anti-CD2 antibodies linked together.

In another aspect, the present invention is directed to a method for expanding a population of TILs comprising contacting the population of TILs with a nanomatrix comprising a colloidal suspension of matrices of polymer chains, wherein the matrices are attached to CD3 agonists and CD28 agonists, wherein the nanomatrix provides activation signals to the population of TILs, thereby activating and inducing the population of TILs to proliferate, wherein each matrix is 1 to 500 nm in length in its largest dimension, and wherein the method does not comprise the use of feeder cells during expansion of the population of TILs.

In some embodiments, the population of TILs contacted with the nanomatrix further comprises tumor cells.

In some embodiments, the population of TILs is isolated from a subject and contacted with the nanomatrix without an additional expansion process of the population of TILs prior to contacting the population of TILs with the nanomatrix.

In some embodiments, the CD3 agonists and the CD28 agonists are attached to the same polymer chains. In some embodiments, the CD3 agonists and the CD28 agonists are attached to different polymer chains. In some embodiments, the CD3 agonists are attached to the nanomatrix at 25 μg per mg of nanomatrix.

In some embodiments, the nanomatrix further comprises magnetic, paramagnetic or superparamagnetic nanocrystals embedded among or within the matrices of polymer chains.

In some embodiments, the matrix of polymer chains comprises a polymer of dextran.

In some embodiments, the polymer chains are colloidal polymer chains.

In some embodiments, the ratio of volume of nanomatrix to volume of TILs is greater than or equal to 1:5. In some embodiments, the ratio of volume of nanomatrix to volume of TILs is greater than or equal to 1:500. In some embodiments, the ratio of volume of nanomatrix to volume of TILs is greater than or equal to 1:10, 1:20, 1:30, 1:40, 1:50, 1:60, 1:70, 1:80, 1:90, 1:100, 1:200, 1:300, 1:400 or 1:500.

In some embodiments, the CD28 agonist is attached to the nanomatrix at 25 μg per mg of nanomatrix. In some embodiments, the agonists are recombinant agonists. In some embodiments, the agonists are antibodies. In some embodiments, the antibodies are humanized antibodies. In some embodiments, the CD3 agonist is OKT3 or UCHT1.

In some embodiments, the methods herein can rescue TIL samples from a previously failed pre-REP expansion. In some embodiments, the TILs to be expanded are from a subject who had previously submitted a sample of TILs for expansion, wherein the previous TIL expansion comprises a pre-REP step and wherein the number of TILs isolated from the pre-REP step was less than 1000 TILs. In some embodiments, the TILs to be expanded are from a subject who had previously submitted a sample of TILs for expansion, wherein the previous TIL expansion comprises a pre-REP step and wherein the fold expansion of TILs isolated from the pre-REP step was less than 5 fold.

In another aspect, the present invention is directed to a method for expanding a population of TILs comprising contacting the population of TILs with a composition comprising a first, a second, and a third soluble monospecific complex, wherein each soluble monospecific complex comprises two antibodies or fragments thereof linked together, wherein each antibody or fragments thereof of each soluble monospecific complex specifically binds to the same antigen on the population of TILs, wherein the first soluble monospecific complex comprises an anti-CD3 antibody, wherein the second soluble monospecific complex comprises an anti-CD28 antibody, and wherein the third soluble monospecific complex comprises an anti-CD2 antibody, and the method does not comprise the use of feeder cells during expansion of the population of TILs.

In some embodiments, the population of TILs contacted with the composition further comprises tumor cells.

In some embodiments, the population of TILs is isolated from a subject and contacted with the composition without an additional expansion process of the population of TILs prior to contacting the population of TILs with the composition.

In some embodiments, the soluble monospecific complexes are at a concentration of 0.2-25 μl/ml.

In some embodiments, the soluble monospecific complexes are tetrameric antibody complexes (TACs). In some embodiments, each TAC comprises two antibodies from a first animal species bound by two antibody molecules from a second species that specifically bind to the Fc portion of the antibodies from the first animal species.

In some embodiments, the anti-CD3 antibody is an OKT3 antibody or an UCHT1 antibody. In some embodiments, the method further comprises contacting the population of TILs with the cytokine IL-2. In some embodiments, the TILs are contacted with the cytokine IL-2 at a time interval selected from the group consisting of 1 day, 2 days, 3 days, 4 days, 5 days, and 6 days. In some embodiments, the final concentration of the cytokine IL-2 is 100 U/ml to 7,000 U/ml.

In some embodiments, the methods herein can rescue TIL samples from a previously failed pre-REP expansion. In some embodiments, the TILs to be expanded are from a subject who had previously submitted a sample of TILs for expansion, wherein the previous TIL expansion comprises a pre-REP step and wherein the number of TILs isolated from the pre-REP step was less than 1000 TILs. In some embodiments, the TILs to be expanded are from a subject who had previously submitted a sample of TILs for expansion, wherein the previous TIL expansion comprises a pre-REP step and wherein the fold expansion of TILs isolated from the pre-REP step was less than 5 fold.

In some embodiments, the TILs are expanded for up to 9, 10, 11, 12, 13, 14, 15, 16, 17, 18, 19, 20, 21, 22, 23, 24, or 25 days. In some embodiments, the TILs are expanded for 9-25 days, 9-21 days, or 9-14 days.

In some embodiments, the TILs are expanded 500 to 500,000-fold. In some embodiments, the population of TILs is expanded from an initial population of TILs of 100 to 100,000 TILs. In some embodiments, the population of TILs is expanded at least 1,500-fold at day 14 of the expansion. In some embodiments, the population of TILs is expanded at most 100,000-fold at day 14 of expansion. In some embodiments, the population of TILs is expanded at least 15,000-fold at day 21 of the expansion. In some embodiments, the population of TILs is expanded at most 500,000-fold at day 21 of expansion.

In some embodiments, members of the population of TILs are genetically modified.

In some embodiments, members of the population of TILs are modified by a gene-regulating system. In some embodiments, the members of the population of TILs are modified using RNA interference. In some embodiments, the members of the population of TILs are modified using a transcription activator-like effector nuclease (TALEN). In some embodiments, the members of the population of TILs are modified using a zinc-finger nuclease. In one embodiment, the members of the population of TILs are modified using an RNA-guided nuclease. In some embodiments, members of the population of TILs are modified using a Cas enzyme and at least one guide RNA. In some embodiments, the Cas enzyme is Cas9.

In some embodiments, members of the population of TILs are modified at one or more genes selected from the group consisting of ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2DA, SMAD2, SOCS1, TANK, TGFBR1, TGFBR2, TIGIT, TNFAIP3, TNIP1, TRAF6, UMPS, WDR6 and ZC3H12A. In some embodiments, members of the population of TILs are modified at one or more genes selected from the group consisting of SOCS1, PTPN2, ZC3H12A, CBLB, RC3H1 and NFKBIA. In some embodiments, the modification at a one or more genes is an insertion, deletion, or mutation of one or more nucleic acids. In some embodiments, the modification at one or more genes results in the reduction or inhibition of expression of the gene and/or function of a protein encoded by the gene. In some embodiments, members of the population of TILs are epigenetically modified. In some embodiments, the epigenetic modification is a histone modification.

In some embodiments, members of the population of TILs are modified at one or more genes selected from the group consisting of ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2DA, SMAD2, SOCS1, TANK, TGFBR1, TGFBR2, TIGIT, TNFAIP3, TNIP1, TRAF6, UMPS, WDR6 and ZC3H12A. the modification at one or more genes is methylation of one or more nucleic acids. In some embodiments, the modification at one or more genes is methylation of one or more nucleic acids. In some embodiments, the modification at one or more genes results in the reduction or inhibition of expression of the gene and/or function of a protein encoded by the gene.

In some embodiments, members of the population of TILs are modified at the SOCS1 gene. In some embodiments, the modification of the SOCS1 gene results in the reduction or inhibition of expression of the gene and/or function of a protein encoded by the gene.

In some embodiments, members of the population of TILs are modified at more than one gene. In some embodiments, members of the population of TILs are modified at two or more genes selected from the group consisting of SOCS1, PTPN2, ZC3H12A, CBLB, RC3H1 and NFKBIA. In some embodiments the two or more genes are selected from the group consisting of ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. In some embodiments, members of the population of TILs are modified at the SOCS1 gene and one or more additional genes. In some embodiments, members of the population of TILs are modified at the SOCS1 gene and one or more additional genes selected from the group consisting of ZC3H12A, PTPN2, CBLB, RC3H1 or NFKBIA. In a specific embodiment, members of the population of TILs are modified at the SOCS1 and ZC3H12A genes. In some embodiments, members of the population of TILs are modified at the SOCS1 and PTPN2 genes. In some embodiments, the modification of the SOCS1 and PTPN2 genes results in the reduction or inhibition of expression of the genes and/or function of proteins encoded by the genes. In some embodiments, members of the population of TILs are modified at the SOCS1 and ZC3H12A genes. In some embodiments, the modification of the SOCS1 and ZC3H12A genes results in the reduction or inhibition of expression of the genes and/or function of proteins encoded by the genes. In some embodiments, members of the population of TILs are modified at the SOCS1 and CBLB genes. In some embodiments, the modification of the SOCS1 and CBLB genes results in the reduction or inhibition of expression of the genes and/or function of proteins encoded by the genes. In some embodiments, members of the population of TILs are modified at the SOCS1 and RC3H genes. In some embodiments, the modification of the SOCS1 and RC3H1 genes results in the reduction or inhibition of expression of the genes and/or function of proteins encoded by the genes. In some embodiments, members of the population of TILs are modified at the SOCS1 and NFKBIA genes. In some embodiments, the modification of the SOCS1 and NFKBIA genes results in the reduction or inhibition of expression of the genes and/or function of proteins encoded by the genes

In some embodiments, the population of TILs is expanded to produce an expanded population of TILs, wherein at least 10% of the expanded population have a central memory T cell phenotype. In some embodiments, the population of TILs is expanded to produce an expanded population of TILs, wherein at least 15% of the expanded population have a central memory T cell phenotype. In some embodiments, the population of TILs is expanded to produce an expanded population of TILs, wherein 5 to 50% of the expanded population have a central memory T cell phenotype at day 14 of expansion. In some embodiments, the population of TILs is expanded to produce an expanded population of TILs, wherein 10 to 25% of the expanded population have a central memory T cell phenotype at day 14 of expansion.

In another aspect, the present invention is directed to a composition comprising an expanded population of TILs produced by any of the methods disclosed herein.

### BRIEF DESCRIPTION OF THE DRAWINGS

The foregoing and other features and advantages of the present invention will be more fully understood from the following detailed description of illustrative embodiments taken in conjunction with the accompanying drawings.

FIG. 1 is a schematic depicting a TIL production workflow using feeder cells.

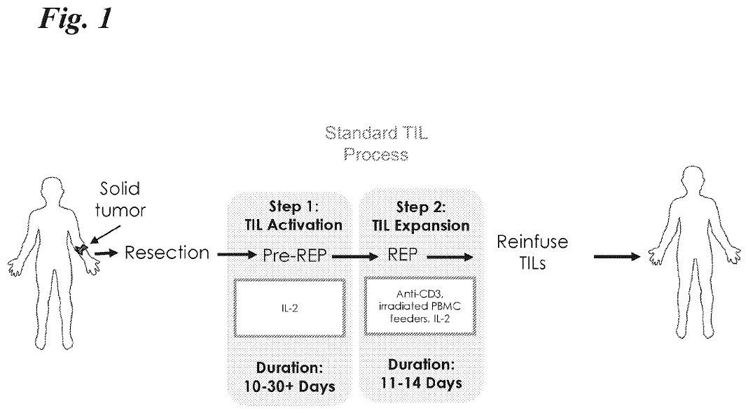

FIG. 2 is a schematic depicting four methods for TIL manufacturing: Method 1 (one-step REP using feeder cells but no pre-REP step (“REP-like”)); Method 2 (using anti-CD3 and anti-CD28 antibody conjugated Dynabeads (“Dynabeads”)); Method 3 (using anti-CD3, anti-CD2, and anti-CD28 antibody-based Tetrameric Antibody Complexes (TACs) (“Stemcell”)); and Method 4 (using anti-CD3 and anti-CD28 antibody-conjugated nanomatrix (“Transact”)).

FIG. 3. FIG. 3A depicts a plot showing the fold expansion of TILs at day 14. FIG. 3B depicts a plot showing the fold expansion of TILs at day 21. In each figure panel each dot represents an independent melanoma donor. Data generated using Method 1 (REP-like), Method 2 (Dynabeads at 1.5 or 0.5×10⁶ beads/well), Method 3 (Stemcell), Method 4 (Transact), and Control (IL-2 alone) are shown.

FIG. 4 depicts a series of FACS analyses providing a gating strategy for cell counts.

FIG. 5. FIG. 5A depicts a plot showing the percentage of live cells in culture that are CD3+ T cells at Days 0, 9, and 14. FIG. 5B depicts a plot showing the percentage of T cells that have a central memory phenotype (Tem, defined as CCR7+ CD45RO+) at day 14. Data generated using Method 1 (REP-like), Method 3 (Stemcell), Method 4 (Transact), and Control (IL-2 alone) are shown.

FIG. 6 depicts a series of FACS analyses showing the percentage of central memory T cell phenotype from three independent donors at day 14.

FIG. 7 depicts bar graphs showing fold expansion at day 14 using FACS counting bead analysis of a TIL pan T cell expansion from three independent donors.

FIG. 8 depicts bar graphs showing fold expansion at day 14 using FACS counting bead analysis of a TIL pan T cell expansion from the combined donors. Data generated using Method 1 (REP-like), Method 2 (Dynabeads at 1.5 or 0.5×10⁶ beads/well) Method 3 (Stemcell), Method 4 (Transact), and Control (IL-2 alone) are shown.

FIG. 9. depicts bar graphs showing extrapolated fold expansion at day 21 using FACS counting bead analysis of a TIL pan T cell expansion from three independent donors. Data generated using Method 1 (REP-like), Method 3 (Stemcell), and Method 4 (Transact) are shown.

FIG. 10 depicts a plot showing fold expansion relative to day 0 of culture at day 14 over time as a function of seeding density.

FIG. 11 depicts bar graphs showing fold induction of expression of IFNγ (FIG. 11A), IL-2 (FIG. 11B), IL-6 (FIG. 11C) and TNFα (FIG. 11D) in TIL donor samples produced using Method 1 (REP-like), Method 3 (Stemcell), and Method 4 (Transact).

FIG. 12 depicts the percent CD45 editing and viability of TILs 8 days post electroporation, where the TILs were expanded using Method 1 (REP-like), Method 3 (Stemcell), or Method 4 (Transact).

FIG. 13 depicts a bar graph showing fold expansion for soluble tetramer and aAPC at day 10 or 11 using Method 1 (REP-like or “REP”), Method 3 (Stemcell or “Stem”), Method 4 (Transact or “Trans”), Method 5 (aAPC-OKT3 or “OKT3”), and Method 6 (aAPC-OKT3-CD86 or “OKT3+CD86”).

FIG. 14 depicts a bar graph showing fold expansion for soluble tetramer and aAPC edited TILs at day 18 (D3399) or day 23 (D6752 and D6755).

FIG. 15 depicts a bar graph showing central memory phenotype for edited TILs at day 18 (D3399) or day 23 (D6752 and D6755).

FIG. 16 depicts a table of editing frequencies at day 18 (D3399) or 23 (D6752 and D6755).

FIG. 17 depicts a bar graph showing TIL tumor fragment extrapolated cell counts at day 14 or 20.

FIG. 18 depicts a bar graph showing central memory phenotype (%) at day 14 or 20.

FIG. 19 depicts a table of editing frequencies at day 14 or 20.

FIG. 20 depicts tables of editing frequencies at day 14 or 20.

FIG. 21 depicts bar graphs showing viability of TILs from different donors prepared from tumor fragments and digests.

FIG. 22 depicts bar graphs showing cell numbers for TILs from different donors prepared from tumor fragments and digests.

### DETAILED DESCRIPTION

The conventional activation and expansion process of TILs necessitates the use of feeder cells, in addition to multiple steps, including at least a separate pre-REP step and REP step. Both requirements render the conventional process time-consuming and expensive. Patients who are in need of immunotherapy using adoptive transfer of TILs often have very poor prognoses and having a population of expanded and differentiated TILs available for therapy more quickly may constitute the difference between life or death.

The necessity of performing a generally slower pre-REP step before a generally faster REP step to achieve an activation and fold expansion of TILs sufficient for therapeutic use is both time-consuming and costly. In certain applications, the pre-REP step of the conventional method can last between 2-6 weeks, with an additional 1-3 weeks of REP. Thus, there is a need to eliminate the pre-REP step and streamline the TIL manufacturing process to one step, with or without the use of feeder cells.

The dependence on feeder cells is especially challenging for at least a few reasons. First, it is very difficult to obtain a viable feeder cell population, because the cells are collected from 3-5 allogeneic donors. The heterogeneous sourcing of feeder cells renders their use non-standardizable, because each population of donor cells must be qualified individually for its ability to expand TILs. Also, due to the inherent variability in the feeder cells, TIL expansion using the feeder cells becomes less reproducible and predictable. Second, when using feeder cells, TILs can only be engineered, or genetically modified, before or after the REP phase, not during, because the TILs cannot be engineered in the presence of the feeder cells. Third, in TIL manufacturing methods involving a REP step, the REP cannot be shortened because the population of expanded TILs cannot be used until the feeder cells die off. Fourth, the use of feeder cells to stimulate TILs results in the inability to wash out and/or remove the stimulating agents. Therefore, in some cases, the pre-REP and REP steps of the conventional process fail to produce the desired number of TILs. For at least the four reasons delineated above, there is a need to eliminate the reliance on feeder cells, which is what has been achieved in the present invention and disclosed herein. Eliminating feeder cells enables enhanced control over the TIL expansion process. For example, the TIL expansion process can be stopped when the number of TILs required is achieved.

In order to provide an improved, faster and simpler method for producing TILs, the present disclosure provides methods for activating and expanding TILs using more streamlined approaches, including one-step approaches, approaches requiring shorter expansion periods, approaches using soluble reagents for stimulation, and approaches without the use of feeder cells. Compositions of expanded populations of TILs are also provided, in addition to isolated populations of expanded TILs enriched in central memory T cell phenotype.

In some aspects, the disclosure relates to methods for activating and expanding TILs in a one-step process without the use of feeder cells, whereby activation occurs through contact with CD3 and CD28 agonists. In certain embodiments, the CD3 and CD28 agonists are bound to a nanomatrix of polymer chains. In certain embodiments, the CD3 and CD28 agonists are antibodies or fragments thereof linked to or complexed with each other. In certain embodiments, the expanded TILs have a higher percentage of cells with a central memory T cell phenotype than TILs isolated using a feeder cell-based method. In certain embodiments, the methods further include the activation of TILs using at least one 4-1BB agonist. In some embodiments, the 4-1BB agonist is 4-1BB ligand.

In some aspects, the disclosure relates to methods for activating and expanding TILs in a one-step process, obviating the need for the pre-REP step and a separate rapid expansion protocol (“REP”) and pre-REP.

Generally, nomenclature used in connection with cell and tissue culture, molecular biology, immunology, microbiology, genetics, and protein and nucleic acid chemistry and hybridization described herein is well-known and commonly used in the art. The methods and techniques provided herein are generally performed according to conventional methods well known in the art and as described in various general and more specific references that are cited and discussed throughout the present specification unless otherwise indicated. Enzymatic reactions and purification techniques are performed according to manufacturer's specifications, as commonly accomplished in the art or as described herein. The nomenclature used in connection with, and the laboratory procedures and techniques of, analytical chemistry, synthetic organic chemistry, and medicinal and pharmaceutical chemistry described herein are well-known and commonly used in the art. Standard techniques are used for chemical syntheses, chemical analyses, pharmaceutical preparation, formulation, and delivery, and treatment of patients.

Unless otherwise defined herein, scientific and technical terms used herein have the meanings that are commonly understood by those of ordinary skill in the art. In the event of any latent ambiguity, definitions provided herein take precedence over any dictionary or extrinsic definition. Unless otherwise required by context, singular terms shall include pluralities and plural terms shall include the singular. The use of “or” means “and/or” unless stated otherwise. The use of the term “including,” as well as other forms, such as “includes” and “included,” is not limiting.

As used herein, the terms “about” and “approximately” refer to a value being within 5% of a given value or range.

As used herein, the phrase “tumor infiltrating lymphocytes” or “TILs” refers to a population of lymphocytes that have left the bloodstream of a subject and migrated into a tumor. TILs include, but are not limited to, CD8⁺ cytotoxic T cells, Th1 and Th17 CD4⁺ T cells, and natural killer (NK) cells. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs and expanded TILs (“REP TILs” or “post-REP TILs”). In some embodiments, primary TILs include tumor reactive T cells that are obtained from peripheral blood of a patient. TIL cell populations can include genetically modified TILs. “TILs” also refers to a population of lymphocytes that have left the blood stream of a subject, have migrated into a tumor and then have departed to again enter the bloodstream.

As used herein, the phrase “population of cells” or “population of TILs” refers to a number of cells or TILs that share common traits. In general, populations generally range from 1×10⁶ to 1×10¹⁰ in number, with different TIL populations comprising different numbers. For example, initial growth of primary TILs in the presence of IL-2 can result in a population of bulk TILs of roughly 1×10⁷ cells. REP expansion is generally done to provide populations of 1.5×10⁹ to 1.5×10¹⁰ cells for infusion.

As used herein, the phrase “expanding a population of TILs” is synonymous with “proliferating a population of TILs” and refers to increasing the number of cells in a TIL population.

As used herein, the phrase “expansion process” refers to the process whereby the number of cells in a TIL population is increased. Processes where TILs are merely isolated or enriched without substantial increase in the number of TILs are not expansion processes.

As used herein, the term “matrix” or “mobile matrix” refers to a discrete, isolatable, three-dimensional lattice-type structure where the backbone of the structure can be flexible or mobile and can be composed of materials, such as polymers and ceramics. Being a three-dimensional structure, a matrix can have a smallest dimension and a largest dimension, such as a length. A mobile matrix may be of collagen, purified proteins, purified peptides, polysaccharides, glycosaminoglycans, or extracellular matrix compositions. A polysaccharide may include for example, cellulose ethers, starch, gum arabic, agarose, dextran, chitosan, hyaluronic acid, pectins, xanthan, guar gum, or alginate. Other polymers may include polyesters, polyethers, polyacrylates, polyacrylamides, polyamines, polyethylene imines, polyquaternium polymers, polyphosphazenes, polyvinylalcohols, polyvinylacetates, polyvinylpyrrolidones, block copolymers, or polyurethanes. The mobile matrix may comprise a polymer of dextran. “Matrices” refers to a collection of more than one matrix.

As used herein, the phrase “largest dimension” in the context of a matrix refers to the longest length of the matrix.

As used herein, the term “agonist” refers to a chemical, a molecule, a macromolecule, a complex of molecules, or a complex of macromolecules that binds to a target, either on the surface of a cell or in soluble form. In certain embodiments, when an agonist binds to a target on the surface of a cell, the agonist activates the target to produce a biological response. Agonists include hormones, neurotransmitters, antibodies, and fragments of antibodies.

As used herein, the term “nanomatrix” refers to a colloidal suspension of more than one matrix of polymer chains. A nanomatrix is a multiphase material that has dimensions of less than 500 nm or structures having nano-scale repeat distances between the different phases that make up the material. Polymers may include polyethylene, polypropylene, polystyrene, polysaccharide, dextran, and other macromolecules, which are composed of many repeated subunits. A nanomatrix may also have embedded within it additional functional compounds, such as magnetic, paramagnetic, or superparamagnetic nanocrystals. In addition, functional moieties, such as ligands or agonists can be covalently attached or bound to the polymer chains for specific applications.

As used herein, the term “dextran” refers to a complex branched glucan, a polysaccharide derived from the condensation of glucose. Dextran chains are of varying lengths, from 3 to 2000 kilodaltons. The polymer main chain consists of α-1,6 glycosidic linkages between glucose monomers, with branches from α-1,3 linkages.

As used herein, the phrase “agonists bound to a nanomatrix” refers to agonists that are covalently attached to the polymer chains that comprise the matrices within the nanomatrix.

As used herein, the phrase “colloidal suspension” refers to a mixture in which one substance, such as a matrix, is suspended throughout another substance, such as a liquid. A colloidal suspension thus has a dispersed phase, i.e., the suspended substance, and a continuous phase, i.e., the medium of suspension, such as a liquid.

As used herein, the phrase “contacting the population of TILs with a nanomatrix” refers to bringing TILs and the nanomatrix together such that the TILs can associate with nanomatrix-bound functional moieties, such as ligands or agonists, or nanomatrix-embedded functional compounds, such as nanocrystals, through ionic, hydrogen-bonding, or other types of physical or chemical interactions.

As used herein, the term “subject” refers to a human being who has a tumor into which a population of lymphocytes that have left the human being's bloodstream have migrated and transformed into TILs. This human being may be a patient in need of immunotherapy involving an expanded population of the patient's own TILs.

As used herein, the term “CD3” refers to the CD3 (cluster of differentiation 3) T cell co-receptor that helps to activate both the cytotoxic T cell (CD8⁺ naïve T cells) and also T helper cells (CD4⁺ naïve T cells). CD3 is a protein complex composed of six distinct polypeptide chains (2 CD3 zeta chains, 2 CD3 epsilon chains, 1 CD3e gamma chain, and 1 CD3 delta chain). These chains associate with the T-cell receptor (TCR) alpha and beta chains (or gamma and delta chains) to generate an activation signal in T lymphocytes. The TCR alpha and beta chains (or gamma and delta chains), and CD3 molecules together constitute the TCR complex. The human CD3E gene is identified by National Center for Biotechnology Information (NCBI) Gene ID 916. An exemplary nucleotide sequence for a human CD3E gene is the NCBI Reference Sequence: NG_007383.1. An exemplary amino acid sequence of a human CD3E polpeptide is provided as SEQ ID NO: 876.

TABLE 1
Sequences of human Cluster of Differentiation
polypeptides and cytokines

| NCBI | SEQ | |
|---|---|---|
| Reference | ID | |
| Sequence No. | NO | SEQUENCE |
| NG_007383.1 | 876 | MQSGTHWRVLGLCLLSVGVWGQDGNEEMGGITQTPYKVSI |
| | | SGTTVILTCPQYPGSEILWOHNDKNIGGDEDDKNIGSDEDHL |
| | | SLKEFSELEQSGYYVCYPRGSKPEDANFYLYLRARVCENCM |
| | | EMDVMSVATIVIVDICITGGLLLLVYYWSKNRKAKAKPVTR |
| | | GAGAGGRQRGQNKERPPPVPNPDYEPIRKGQRDLYSGLNQR |
| | | RI |
| NG_029618.1 | 877 | MLRLLLALNLFPSIQVTGNKILVKQSPMLVAYDNAVNLSCK |
| | | YSYNLFSREFRASLHKGLDSAVEVCVVYGNYSQQLQVYSKT |
| | | GFNCDGKLGNESVTFYLQNLYVNQTDIYFCKIEVMYPPPYLD |
| | | NEKSNGTIIHVKGKHLCPSPLFPGPSKPFWVLVVVGGVLACY |
| | | SLLVTVAFIIFWVRSKRSRLLHSDYMNMTPRRPGPTRKHYQP |
| | | YAPPRDFAAYRS |
| NG_050908.1 | 878 | MSFPCKFVASFLLIFNVSSKGAVSKEITNALETWGALGQDINL |
| | | DIPSFQMSDDIDDIKWEKTSDKKKIAQFRKEKETFKEKDTYK |
| | | LFKNGTLKIKHLKTDDQDIYKVSIYDTKGKNVLEKIFDLKIQE |
| | | RVSKPKISWTCINTTLTCEVMNGTDPELNLYQDGKHLKLSQR |
| | | VITHKWTTSLSAKFKCTAGNKVSKESSVEPVSCPEKGLDIYLI |
| | | IGICGGGSLLMVFVALLVFYITKRKKQRSRRNDEELETRAHR |
| | | VATEERGRKPHQIPASTPQNPATSQHPPPPPGHRSQAPSHRPP |
| | | PPGHRVQHQPQKRPPAPSGTQVHQQKGPPLPRPRVQPKPPHG |
| | | AAENSLSPSSN |
| NG_016779.1 | 879 | MYRMQLLSCIALSLALVTNSAPTSSSTKKTQLQLEHLLLDLQ |
| | | MILNGINNYKNPKLTRMLTFKFYMPKKATELKHLQCLEEELK |
| | | PLEEVLNLAQSKNFHLRPRDLISNINVIVLELKGSETTFMCE |
| | | YADETATIVEFLNRWITFCQSIISTLT |
| NP_001552.2 | 880 | MGNSCYNIVATLLLVLNFERTRSLQDPCSNCPAGTFCDNNRN |
| | | QICSPCPPNSFSSAGGQRTCDICRQCKGVFRTRKECSSTSNA |
| | | ECDCTPGFHCLGAGCSMCEQDCKQGQELTKKGCKDCCFGTF |
| | | NDQKRGICRPWTNCSLDGKSVLVNGTKERDVVCGPSPADLS |
| | | PGASSVTPPAPAREPGHSPQIISFFLALTSTALLFLLFFLT |
| | | LRFSVVKRGRKKLLYIFKQPFMRPVQTTQEEDGCSCRFPEE |
| | | EEGGCEL |
| NP_003802.1 | 881 | MEYASDASLDPEAPWPPAPRARACRVLPWALVAGLLLLLLL |
| | | AAACAVFLACPWAVSGARASPGSAASPRLREGPELSPDDPA |
| | | GLLDLRQGMFAQLVAQNVLLIDGPLSWYSDPGLAGVSLTGG |
| | | LSYKEDTKELVVAKAGVYYVFFQLELRRVVAGEGSGSVSLA |
| | | LHLQPLRSAAGAAALALTVDLPPASSEARNSAFGFQGRLLHL |
| | | SAGQRLGVHLHTEARARHAWQLTQGATVLGLFRVTPEIPAG |
| | | LPSPRSE |

In Table 1, the presumed leader sequences for proteins that have them are shown as underlined.

As used herein, the term “CD28” refers to cluster of differentiation 28, which is one of the proteins expressed on T cells that provides co-stimulatory signals required for T cell activation and survival. T cell stimulation through CD28 in addition to the T-cell receptor (TCR) can provide a potent signal for the production of various cytokines, such as interleukins. CD28 is the receptor for CD80 and CD86 proteins. When activated by Toll-like receptor ligands, CD80 expression is upregulated in antigen-presenting cells (APCs). The human CD28 gene is identified by NCBI Gene ID 940. An exemplary nucleotide sequence for a human CD28 gene is the NCBI Reference Sequence: NG_029618.1. An exemplary amino acid sequence of a human CD28 polypeptide is provided as SEQ ID NO: 877.

As used herein, the term “CD2” refers to cluster of differentiation 2, which is a cell adhesion molecule found on the surface of T cells and natural killer (NK) cells. CD2 interacts with other adhesion molecules and acts as a co-stimulatory molecule on T and NK cells. The human CD2 gene is identified by NCBI Gene ID 914. An exemplary nucleotide sequence for a human CD2 gene is the NCBI Reference Sequence: NG_050908.1. An exemplary amino acid sequence of a human CD2 polypeptide is provided as SEQ ID NO: 878.

As used herein, the term “4-1BB” refers to CD137, which is a T cell costimulator. An exemplary nucleotide sequence for a human 4-1BB gene is the NCBI Reference Sequence: NC_000001.11. An exemplary amino acid sequence of a human 4-1BB is the NCBI Reference Sequence: NP_001552.2 (SEQ ID NO: 880).

As used herein, the term “4-1BB ligand” refers to a type 2 transmembrane glycoprotein that is expressed on activated T-lymphocytes and binds 4-1BB. An exemplary nucleotide sequence for a human 4-1BB ligand gene is the NCBI Reference Sequence: NC_000019.10. An exemplary amino acid sequence of a human 4-1BB ligand is the NCBI Reference Sequence: AAA53134.1 (SEQ ID NO: 881).

As used herein, the term “fragment” used in association with agonist or antibody, refers to a fragment of the agonist or antibody that retains the ability to specifically bind to an antigen. Examples of fragments of antibodies include (i) an Fab fragment, a monovalent fragment consisting of the VL, VH, CL and CH1 domains; (ii) an F(ab′)2 fragment, a bivalent fragment comprising two Fab fragments linked by a disulfide bridge at the hinge region; (iii) an Fd fragment consisting of the VH and CH1 domains; (iv) an Fv fragment consisting of the VL and VH domains of a single arm of an antibody; (v) a dAb fragment, which comprises a single variable domain; and (vi) an isolated complementarity determining region (CDR). Furthermore, although the two domains of the Fv fragment, VL and VH, are encoded by separate genes, they can be joined, using recombinant methods, by a synthetic linker that enables them to be made as a single protein chain in which the VL and VH regions pair to form monovalent molecules (known as single chain Fv (scFv)). Such single chain antibodies are also intended to be encompassed within the term “antigen-binding portion” of an antibody. Other forms of single chain antibodies, such as diabodies are also encompassed. In addition, single chain antibodies also include “linear antibodies” comprising a pair of tandem Fv segments (VH-CH1-VH-CH1), which, together with complementary light chain polypeptides, form a pair of antigen binding regions.

The term “antibody” refers to an immunoglobulin (Ig) molecule, which is generally comprised of four polypeptide chains, two heavy (H) chains and two light (L) chains, or a functional fragment, mutant, variant, or derivative thereof, that retains the epitope binding features of an Ig molecule. Such fragment, mutant, variant, or derivative antibody formats are known in the art. In an embodiment of a full-length antibody, each heavy chain is comprised of a heavy chain variable region (VH) and a heavy chain constant region (CH). The heavy chain variable region (domain) is also designated as VDH in this disclosure. The CH is comprised of three domains, CH1, CH2 and CH3. Each light chain is comprised of a light chain variable region (VL) and a light chain constant region (CL). The CL is comprised of a single CL domain. The light chain variable region (domain) is also designated as VDL in this disclosure. The VH and VL can be further subdivided into regions of hypervariability, termed complementarity determining regions (CDRs), interspersed with regions that are more conserved, termed framework regions (FRs). Generally, each VH and VL is composed of three CDRs and four FRs, arranged from amino-terminus to carboxy-terminus in the following order: FR1, CDR1, FR2, CDR2, FR3, CDR3, and FR4. Immunoglobulin molecules can be of any type (e.g., IgG, IgE, IgM, IgD, IgA and IgY), class (e.g., IgG1, IgG2, IgG3, IgG4, IgA1 and IgA2), or subclass.

The phrases “selective binding” or “selectively binds” as used herein refer to agonist binding to an epitope on a predetermined antigen. Typically, the agonist binds with an affinity (K_{D}) of approximately less than 10⁻⁵ M, such as approximately less than 10⁻⁶ M, 10⁻⁷ M, 10⁻⁸M, 10⁻⁹ M or 10⁻¹⁰ M or even lower.

The term “K_{D}” as used herein refers to the dissociation equilibrium constant of a particular agonist-antigen interaction. Typically, the agonists described herein bind to a target with a dissociation equilibrium constant (K_{D}) of less than approximately 10⁻⁶ M, 10⁻⁷ M, 10⁻⁸ M, 10⁻⁹ M or 10⁻¹⁰ M or even lower, for example, as determined using surface plasmon resonance (SPR) technology in a Biacore instrument using the agonist as the ligand and the target as the analyte, and bind to a target protein with an affinity corresponding to a K_{D} that is at least ten-fold lower, such as at least 100-fold lower, for instance at least 1000-fold lower, such as at least 10,000-fold lower, for instance at least 100,000-fold lower than its affinity for binding to a non-specific antigen (e.g., BSA, casein) other than the predetermined antigen or a closely-related antigen. The amount with which the affinity is lower is dependent on the K_{D} of the agonist, so that when the K_{D} of the agonist is very low (that is, the agonist is highly specific), the amount with which the affinity for the antigen is lower than the affinity for a non-specific antigen may be at least 10,000-fold.

The term “k_{d}” (sec⁻¹) as used herein refers to the dissociation rate constant of a particular agonist-antigen interaction. Said value is also referred to as the k_{off} value.

The term “kₐ” (M⁻¹×sec⁻¹) as used herein refers to the association rate constant of a particular agonist-antigen interaction.

The term “KD” (M) as used herein refers to the dissociation equilibrium constant of a particular agonist-antigen interaction.

The term “K_{A}” (M⁻¹) as used herein refers to the association equilibrium constant of a particular agonist-antigen interaction and is obtained by dividing the kₐ by the k_{d}.

As used herein, the phrase “activation signal” refers to one or more non-endogenous stimuli that cause T cells to become activated. In the endogenous process, T cells become activated when they are presented with peptide antigens by MHC class II molecules, which are expressed on the surface of antigen-presenting cells (APCs). Once activated, the T cells divide rapidly and secrete cytokines that regulate or assist the immune response. The endogenous T cell activation process involves at least (a) activation of the TCR complex, which involves CD3, and (b) co-stimulation of CD28 or 4-1BB by proteins on the APC surface. It is known in the art that the endogenous activation of T cells can be simulated by stimulation of T cells by CD3, CD28 or 4-1BB agonists (e.g. antibodies). Thus, CD3, CD28 and/or 4-1BB can together provide an activation signal to T cells.

As used herein, the phrase “activating and inducing the population of TILs to proliferate” refers to the process of subjecting a population of TILs to activation signals, so that the TILs increase in number or proliferate and begin producing cytokines (activated TILs) to boost the immune response.

As used herein, the term “nanocrystal” refers to a material particle having at least one dimension smaller than 100 nm, based on quantum dots and composed of atoms in either a single- or poly-crystalline arrangement. The size of nanocrystals distinguishes them from larger crystals.

As used herein, the phrase “magnetic, paramagnetic, or superparamagnetic nanocrystals” refers to nanocrystals that can be manipulated using magnetic fields. Such nanocrystals commonly consist of at least one component that is a magnetic material, such as iron, nickel, or cobalt.

As used herein, the phrase “tumor cells” or “cancer cells” refers to cells that divide in an uncontrolled manner, forming solid tumors or flooding the blood with abnormal cells. Healthy cells stop dividing when there is no longer a need for more daughter cells, but tumor cells or cancer cells continue to produce copies. They are also able to spread from one part of the body to another in a process known as metastasis. Tumor cells can be isolated from a number of cancer types including bladder cancer, breast cancer, cervical cancer, colon and rectal cancer, endometrial cancer, kidney cancer, lip and oral cancer, liver cancer, melanoma, mesothelioma, lung cancer, non-small cell lung cancer, head and neck cancer, neuroblastoma, glioblastoma multiforme, nonmelanoma skin cancer, ovarian cancer, pancreatic cancer, prostate cancer, sarcoma, small cell lung cancer, and thyroid cancer. Tumor cells can be isolated from primary tumors and metastases.

As used herein, the phrase “tumor sample” refers to tumor cells isolated from a subject. In certain embodiments, a tumor sample is at least a portion of a solid tumor that is isolated in its entirety or in part from a subject or patient having a tumor. A tumor sample can be isolated from a number of cancer types, including bladder cancer, breast cancer, cervical cancer, colon and rectal cancer, endometrial cancer, kidney cancer, lip and oral cancer, liver cancer, melanoma, mesothelioma, lung cancer, non-small cell lung cancer, head and neck cancer, neuroblastoma, glioblastoma multiforme, nonmelanoma skin cancer, ovarian cancer, pancreatic cancer, prostate cancer, sarcoma, small cell lung cancer, and thyroid cancer. Tumor samples can be isolated from primary tumors and metastases.

As used herein, the phrase “disaggregated tumor sample” refers to a tumor sample that has been fragmented into “tumor fragments”. The fragmentation may be physical fragmentation, mechanical fragmentation, ultrasonic fragmentation, enzymatic fragmentation, or any combinations thereof. The fragmentation may be done mechanically and optionally be followed by enzymatic digestion of the tumor fragments into a single cell suspension. Mechanical disaggregation methods may include chopping or slicing the tumor into smaller tumor fragments, while enzymatic disaggregation methods may include treating the tumor fragments with specific enzymes, such as proteases.

In some embodiments, the methods herein can rescue TIL samples from a previously failed pre-REP expansion. In certain embodiments, the tumor sample is isolated from a subject who has previously had a sample subject to a TIL expansion technique. In some embodiments, the previous TIL expansion technique comprised a pre-REP expansion. In some embodiments, the pre-REP expansion comprises administration of IL-2 to a disaggregated tumor sample from the subject. In some embodiments, in the pre-REP expansion the only immunomodulator administered to the tumor sample or the TILs expanded from the tumor sample is IL-2. In some embodiments, the previous TIL expansion technique failed. In some embodiments, a TIL expansion technique fails when it does not expand an adequate number of TILs. In some embodiments, an adequate number of TILs is greater than 1000, 5000, 10,000, 20,000, 30,000, 40,000, 50,000, 60,000, 70,000, 80,000, 90,000 or 100,000 TILs. In some embodiments, a TIL expansion technique fails when it does not induce an adequate fold expansion of the TILs. In some embodiments, an adequate fold expansion of TILs is greater than 50, 100, 1000, 2000, 3000, 4000, 5000, 6000, 7000, 8000, 9000 or 10,000 fold expansion. In some embodiments, a portion of the same tumor sample is used in the previous TIL expansion technique and the TIL expansion methods disclosed, herein. In some embodiments, two distinct samples are isolated from the same subject. In some embodiments, the methods described herein are able to provide greater numbers or fold expansion of TILs than the previous expansion technique. In some embodiments, the methods described herein are able to provide a clinically useful number of TILs, wherein the previous expansion technique was unable to provide that number of TILs.

As used herein, the phrase “T cell receptor agonist” or “TCR agonist” refers to an agonist of the T cell receptor complex. Suitable TCR agonists include, without limitation, CD3 agonists (e.g., anti-CD3 antibodies).

As used herein, the term “medium” refers to a liquid or gel designed to support the survival, growth, and/or proliferation of cells in an artificial environment. A medium generally comprises a defined set of components. Such components may include an energy source, growth factors, hormones, stimulants, activators, sugars, salts, vitamins, and/or amino acids, and/or a combination of these.

As used herein, the phrase “components of the medium are maintained” refers to a medium comprising a defined set of components, such as particular stimulants and activators, where the identity of the components remains constant, but the concentration of one or more of the components may be varied. In certain embodiments, the concentration of one or more components in the media varies over time while the cells are cultured in the media. However, when the media is changed the fresh media has the same components for each change.

As used herein, the phrase “feeder cell” refers to cells used to provide extracellular secretions that help another cell type proliferate. In certain embodiments, the feeder cells referred to herein are peripheral blood mononuclear cell (PBMC) or an antigen-presenting cell (APC).

As used herein, the phrase “recombinant agonist” refers to an agonist protein that is encoded by a recombinant gene, which has been cloned in a system that supports expression of the gene and translation of mRNA. The recombinant gene is designed to be under the control of a well characterized promoter and to express the target agonist protein within the chosen host cell to achieve high-level protein expression. Modification of the gene by recombinant DNA technology can lead to expression of a mutant protein or a large quantity of protein.

As use herein, the phrase “colloidal polymer chains” refers to polymer chains that when linked to each other through covalent bonds or other physical or chemical interactions can form colloidal suspensions.

As used herein, the phrase “specifically bind” refers to a protein complex, such as an agonist, antagonist, antibody or soluble monospecific complex, interacting with high specificity with a particular antigen, as compared with other antigens for which the complex has a lower affinity to associate. The specific binding interaction can be mediated through ionic bonds, hydrogen bonds, or other types of chemical or physical associations. In certain embodiments, a protein complex specifically binds a particular antigen when it recognizes its target antigen in a complex mixture of proteins and/or macromolecules. Two or more agonist, antagonist, antibody or soluble monospecific complex “bind to the same epitope” if the agonists cross-compete (one prevents the binding or modulating effect of the other).

As used herein, the phrase “central memory T cell phenotype” refers to a subset of T cells that in the human are CD45RO+ and express CCR7 (CCR7^{hi}) and CD62L (CD62^{hi}). The surface phenotype of central memory T cells also includes TCR, CD3, CD127 (IL-7R). Central memory T cells primarily secret IL-2 and CD40L as effector molecules after TCR triggering. Central memory T cells are predominant in the CD4 compartment in blood, and in human beings are proportionally enriched in lymph nodes and tonsils.

As used herein, the phrase “anti-CD3 antibody” refers to an antibody or variant thereof, e.g., a monoclonal antibody, and includes human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UCHT1 clone, also known as T3 and CD3c. Other anti-CD3 antibodies include, for example, otelixizumab, teplizumab, and visilizumab.

As used herein, the phrase “anti-CD28 antibody” refers to an antibody or variant thereof, e.g., a monoclonal antibody, and includes human, humanized, chimeric or murine antibodies which are directed against the CD28 receptor in the T cell antigen receptor of mature T cells.

As used herein, the phrase “anti-4-1BB antibody” refers to an antibody or variant thereof, e.g., a monoclonal antibody, and includes human, humanized, chimeric or murine antibodies which are directed against 4-1BB. In some embodiments, an anti-4-1BB antibody can be utilized as a 4-1BB ligand.

As used herein, the phrase “anti-CD2 antibody” refers to an antibody or variant thereof, e.g., a monoclonal antibody, and includes human, humanized, chimeric or murine antibodies which are directed against the CD2 receptor in the T cell antigen receptor of mature T cells.

As used herein, the term “OKT-3” (also referred to herein as “OKT3”) refers to the anti-CD3 antibody produced by Miltenyi Biotech, Inc., San Diego, Calif., USA) and or biosimilar or variant thereof (e.g., a humanized, chimeric, or affinity matured variant). A hybridoma capable of producing OKT-3 is available in the American Type Culture Collection and assigned the ATCC accession number CRL 8001. A hybridoma capable of producing OKT-3 is available in the European Collection of Authenticated Cell Cultures (ECACC) and assigned Catalogue No. 86022706.

As used herein, the term “UCHT1” refers to the anti-CD3 antibody described in Beverley and Callard (1981) Eur. J. Immunol. 11: 329-334, and or biosimilar or variant thereof (e.g., a humanized, chimeric, or affinity matured variant). A hybridoma capable of producing an exemplary UCHT1 is available from Creative Diagnostics, Shirley, NY, USA, and assigned Catalogue No. CSC-H3068.

As used herein, the phrase “tetrameric antibody complex” or “TAC” refers to a protein complex comprising two antibodies that act as the first and second agonists that are linked by one or two linker antibodies that bind the antibodies acting as first and second agonists. The linker antibodies may bind the constant region of the agonist antibodies, and where the constant regions are of different isotypes, a bi-specific antibody with one binding region for each isotype may also be used. Support for these complexes can also be found in U.S. Pat. No. 4,868,109, incorporated by reference herein in its entirety. In other embodiments, the antibodies, or antigen binding fragments thereof, that act as first and second ligands may be covalently or non-covalently bound by one or more linker molecules. Non-limiting examples of such linker molecules include avidin or streptavidin, which may be used to join biotinylated antibodies, such as antibodies with biotin moieties in the Fc region. In additional embodiments, tetrameric antibody complexes may be used as a mixture of complexes. This includes use of more than one species of complex in a mixture of complexes, wherein the complexes of the entire mixture can contact more than two different ligands.

As used herein, the phrase “RNA-guided nuclease” refers to a nucleic acid/protein complex based on naturally occurring Type II CRISPR-Cas systems, that is a programmable endonuclease that can be used to perform targeted genome editing. RNA-guided nucleases consist of two components: a short ˜100 nucleotide guide RNA (gRNA) that uses 20 variable nucleotides at its 5′ end to base pair with a target genomic DNA sequence and a nuclease, e.g., the Cas9 endonuclease, that cleaves the target DNA.

As used herein, the term “Cas9” refers to CRISPR associated protein 9, a protein that plays a vital role in the immunological defense of certain bacteria against DNA viruses, and which is heavily utilized in genetic engineering applications. Cas9 is an RNA-guided DNA endonuclease enzyme associated with the CRISPR (clustered regularly interspaced short palindromic repeats) adaptive immunity system in Streptococcus pyogenes. Cas9 can interrogate sections of DNA by checking for sites complementary to a guide RNA (gRNA). If the DNA substrate is complementary to the gRNA, Cas9 cleaves the DNA. Because the target specificity of Cas9 stems from the gRNA:DNA complementarity and not modifications to the protein itself (like TALENs and Zinc-fingers), engineering Cas9 to target new DNA is straightforward. Versions of Cas9 that bind but do not cleave cognate DNA can be used to locate transcriptional activators or repressors to specific DNA sequences in order to control transcriptional activation and repression. Native Cas9 requires a guide RNA composed of two disparate RNAs that associate, the CRISPR RNA (crRNA) and the trans-activating crRNA (tracrRNA). Cas9 targeting has been simplified through the engineering of a chimeric single guide RNA.

As used herein, the phrase “dead Cas9” or “dCas9” refers to Cas9 endonuclease Dead, which is a mutant form of Cas9 whose endonuclease activity is removed through point mutations in its endonuclease domains. Similar to its unmutated form, dCas9 is used in CRISPR systems along with gRNAs to target specific genes or nucleotides complementary to the gRNA with PAM sequences that allow Cas9 to bind. Cas9 ordinarily has 2 endonuclease domains called the RuvC and HNH domains. The point mutations D10A and H840A change two important residues for endonuclease activity that ultimately results in its deactivation. Although dCas9 lacks endonuclease activity, it is still capable of binding to its guide RNA and the DNA strand that is being targeted because such binding is managed by other domains. This alone is often enough to attenuate if not outright block transcription of the targeted gene if the gRNA positions dCas9 in a way that prevents transcriptional factors and RNA polymerase from accessing the DNA. However, this ability to bind DNA can also be exploited for activation since dCas9 has modifiable regions, typically the N and C terminus of the protein, that can be used to attach transcriptional activators.

As used herein, the term “cytokine” refers to abroad category of small proteins (about 5-20 kDa in size) that are important in cell signaling. Cytokines are peptides and cannot cross the lipid bilayer of cells to enter the cytoplasm. Cytokines have been shown to be involved in autocrine signaling, paracrine signaling, and endocrine signaling as immunomodulating agents. Cytokines include chemokines, interferons, interleukins, lymphokines, and tumor necrosis factors, but generally not hormones or growth factors, although there is some overlap in terminology. Cytokines are produced by a broad range of cells, including immune cells like macrophages, B lymphocytes, T lymphocytes, and mast cells, as well as endothelial cells, fibroblasts, and various stromal cells. Cytokines generally act through binding to cell-surface receptors and are especially important in the immune response, since they are involved in regulating the maturation, growth, and responsiveness of particular cell populations.

As used herein, the phrase “T cell-stimulating cytokine” refers to a cytokine that stimulates and/or activates T cell lymphocytes. In some embodiments, the T-cell stimulating cytokine is IL-2.

As used herein, the term “IL-2” (also referred to herein as “IL2”) refers to the cytokine and T cell growth factor known as interleukin-2, and includes all forms of IL-2, including human and mammalian forms, forms with conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated herein by reference in their entireties. The term IL-2 encompasses human, recombinant forms of IL-2, such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The term IL-2 also encompasses pegylated forms of IL-2, including the pegylated IL-2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated herein by reference in their entireties. Alternative forms of conjugated IL-2 suitable for use in the invention are described in U.S. Pat. Nos. 4,766,106, 5,206,344, 5,089,261 and 4,902,502, the disclosures of which are incorporated herein by reference in their entireties. Formulations of IL-2 suitable for use in the invention are described in U.S. Pat. No. 6,706,289, the disclosure of which is incorporated herein by reference in its entirety. The human IL2 gene is identified by NCBI Gene ID 3558. An exemplary nucleotide sequence for a human IL2 gene is the NCBI Reference Sequence: NG_016779.1. An exemplary amino acid sequence of a human IL-2 polypeptide is provided as SEQ ID NO: 879.

As used herein, the phrase “soluble monospecific complex” refers to a complex that comprises two binding proteins that are linked, either directly or indirectly, to each other and bind to the same antigen. The two binding proteins are soluble and not immobilized on a surface, particle, or bead.

Furthermore, in accordance with the present disclosure there may be employed conventional molecular biology, microbiology, and recombinant DNA techniques within the skill of the art. Such techniques are explained fully in the literature. See, e.g., Sambrook, Fritsch & Maniatis, Molecular Cloning: A Laboratory Manual, Second Edition (1989) Cold Spring Harbor Laboratory Press, Cold Spring Harbor, N.Y. (herein “Sambrook et al., 1989”); DNA Cloning: A Practical Approach, Volumes I and II (D. N. Glover ed. 1985); Oligonucleotide Synthesis (M. J. Gait ed. 1984); Nucleic Acid Hybridization [B. D. Hames & S. J. Higgins eds. (1985)]; Transcription And Translation [B. D. Hames & S. J. Higgins, eds. (1984)]; Animal Cell Culture [R. I. Freshney, ed. (1986)]; Immobilized Cells And Enzymes [IRL Press, (1986)]; B. Perbal, A Practical Guide To Molecular Cloning (1984); F. M. Ausubel et al. (eds.), Current Protocols in Molecular Biology, John Wiley & Sons, Inc. (1994). Each of these references are incorporated by reference herein in its entirety.

Unless otherwise stated, sequence identity/similarity values provided herein refer to the value obtained using the BLAST 2.0 suite of programs using default parameters (Altschul, et al., (1997) Nucleic Acids Res. 25:3389-402, incorporated by reference herein in its entirety).

As used herein, “nucleic-acid targeting sequence” and “nucleic-acid binding sequence” are used interchangeably and refer to sequences that bind and/or target nucleic acids.

As used herein, “sequence identity” or “identity” in the context of two nucleic acid or polypeptide sequences includes reference to the residues in the two sequences, which are the same when aligned for maximum correspondence over a specified comparison window. When percentage of sequence identity is used in reference to proteins, it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. Where sequences differ in conservative substitutions, the percent sequence identity may be adjusted upwards to correct for the conservative nature of the substitution. Sequences, which differ by such conservative substitutions, are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., according to the algorithm of Meyers and Miller, (1988) Computer Applic. Biol. Sci. 4:11-17, e.g., as implemented in the program PC/GENE (Intelligenetics, Mountain View, Calif., USA). Each of these references are incorporated by reference herein in its entirety.

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity.

The term “substantial identity” or “substantially identical” in the context of polynucleotide sequences means that a polynucleotide comprises a sequence that has between 50-100% sequence identity, preferably at least 50% sequence identity, preferably at least 60% sequence identity, preferably at least 70%, more preferably at least 80%, more preferably at least 90% and most preferably at least 95%, compared to a reference sequence using one of the alignment programs described using standard parameters. One of skill will recognize that these values can be appropriately adjusted to determine corresponding identity of proteins encoded by two nucleotide sequences by taking into account codon degeneracy, amino acid similarity, reading frame positioning and the like. Substantial identity of amino acid sequences for these purposes normally means sequence identity of between 55-100%, preferably at least 55%, preferably at least 60%, more preferably at least 70%, 80%, 90% and most preferably at least 95%.

### I. Tumor Infiltrating Lymphocytes (TILs)

Tumor infiltrating lymphocytes or TILs are a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, and natural killer (NK) cells. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein.

TILs can generally be defined either biochemically, using cell surface markers, or functionally, by their ability to infiltrate tumors and effect treatment. TILs can be generally categorized as expressing one or more of the following biomarkers: CD4, CD8, TCR αβ, TCRγδ, CD27, CD28, CD56, CCR7, CD45RA, CD45RO, CD95, PD-1, and CD25. Additionally, and alternatively, TILs can be functionally defined by their ability to infiltrate solid tumors upon reintroduction into a patient. TILS may further be characterized by potency; for example, TILS may be considered potent if, for instance, interferon gamma (IFNγ) release is greater than about 50 pg/mL, greater than about 100 pg/mL, greater than about 150 pg/mL, or greater than about 200 pg/mL upon TCR stimulation.

Adoptive cell therapy utilizing TILs cultured ex vivo by conventional TIL manufacturing processes involves at least two steps, namely at least one rapid expansion protocol (REP) step subsequent to a pre-REP step. Adoptive cell therapy has resulted in successful therapy following host immunosuppression in patients with melanoma. Current infusion acceptance parameters rely on readouts of the composition of TILs (e.g., CD28, CD8, or CD4 positivity) and on the numerical folds of expansion and viability of the REP product.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention may utilize a lymphodepletion step (sometimes also referred to as “immunosuppressive conditioning”) on the patient prior to the introduction of the TILs of the invention. In some embodiments, a lymphodepletion step is not used.

### A. Expansion of TILs

As generally outlined herein, TILs are generally taken from a patient sample and manipulated to expand their number prior to transplant into a patient. In some embodiments, the TILs may be genetically manipulated as discussed below. In general, TILs are initially obtained from a patient tumor sample (“primary TILs”) and then expanded into a larger population for further manipulation as described herein, optionally cryopreserved and re-stimulated, and optionally evaluated for phenotype and metabolic parameters as an indication of TIL health.

A patient tumor sample may be obtained using methods known in the art, generally via surgical resection, needle biopsy, or other means for obtaining a sample that contains a mixture of tumor and TIL cells. In general, the tumor sample may be from any solid tumor, including primary tumors, invasive tumors or metastases. The solid tumor may be of any cancer type, including, but not limited to, bladder cancer, breast cancer, cervical cancer, colon and rectal cancer, endometrial cancer, kidney cancer, lip and oral cancer, liver cancer, melanoma, mesothelioma, lung cancer, non-small cell lung cancer, head and neck cancer, neuroblastoma, glioblastoma multiforme, nonmelanoma skin cancer, ovarian cancer, pancreatic cancer, prostate cancer, sarcoma, small cell lung cancer, and thyroid cancer). In some embodiments, useful TILs are obtained from malignant melanoma tumors, as these have been reported to have particularly high levels of TILs. Primary melanoma tumors or metastases thereof can be used to obtain TILs.

In some embodiments, a solid tumor is an abnormal mass of tissue that usually does not contain cysts or liquid areas. Solid tumors may be benign or malignant. Solid tumor cancer refers to malignant, neoplastic, or cancerous solid tumors. Solid tumor cancers include, but are not limited to, lymphoma, sarcoma, breast (including triple negative breast cancer), pancreatic, prostate, colon, rectum, bladder, lung (including non small cell lung carcinoma (NSCLC)), brain, renal, stomach, and skin (including but not limited to squamous cell carcinoma, basal cell carcinoma, and melanoma). In some embodiments, the cancer is selected from cervical cancer, head and neck cancer (including, for example, head and neck squamous cell carcinoma (HNSCC)) neuroblastoma, glioblastoma, glioblastoma multiforme, liver cancer, ovarian cancer, sarcoma, pancreatic cancer, bladder cancer, breast cancer, triple negative breast cancer, and non-small cell lung carcinoma. The tissue structure of solid tumors includes interdependent tissue compartments, including the parenchyma (cancer cells) and the supporting stromal cells in which the cancer cells are dispersed and which may provide a supporting microenvironment.

Once obtained, the tumor sample is generally fragmented using sharp dissection into small pieces of from about 1 to about 8 mm³, or from about 0.5 to about 4 mm³ with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicin, 30 units/mL of DNase and 1.0 mg/mL of collagenase), followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated herein by reference in its entirety. Any of the foregoing methods may be used in any of the embodiments described herein for methods of expanding TILs or methods treating a cancer.

In general, the harvested cell suspension is called a “primary cell population” or a “freshly harvested” cell population. In some embodiments, fragmentation includes physical fragmentation, including for example, dissection as well as digestion. In some embodiments, the fragmentation is physical fragmentation. In some embodiments, the fragmentation is by dissection. In some embodiments, the fragmentation is by digestion. In some embodiments, TILs can be initially cultured from enzymatic tumor digests and tumor fragments obtained from patients.

In some embodiments, where the tumor is a solid tumor, the tumor undergoes physical fragmentation after the tumor sample is obtained. In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the tumor is fragmented and 10, 20, 30, 40 or more fragments or pieces are placed in each container for the first expansion. In some embodiments, the tumor is fragmented and 30 or 40 fragments or pieces are placed in each container for the first expansion. In some embodiments, the tumor is fragmented and 40 fragments or pieces are placed in each container for the first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is from about 1 mm³ and 8 mm³. In some embodiments, the tumor fragment is from about 0.5 mm³ and 4 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³.

In some embodiments, the TILs are obtained from tumor digests. In some embodiments, tumor digests are generated by incubation in enzyme media, for example, but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, Calif.). After placing the tumor in enzyme media, the tumor can be mechanically dissociated for approximately 1 minute. The solution can then be incubated for 30 minutes at 37° C. in 5% CO₂ and can then be mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue are present, one or two additional mechanical dissociations can be applied to the sample, with or without 30 additional minutes of incubation at 37° C. in 5% CO₂. In some embodiments, at the end of the final incubation if the cell suspension contains a large number of red blood cells or dead cells, a density gradient separation using FICOLL can be performed to remove these cells.

In some embodiments, cells can be optionally frozen or cryopreserved after sample harvest and stored frozen prior to entry into the expansion phase.

### 1. Overview of Conventional Methods for TIL Expansion

In conventional methods of activating and expanding TILs, a multi-step process is employed, in addition to the use of feeder cells. This multi-step process includes at least one rapid expansion protocol (REP) step, preceded by a separate pre-REP step.

a. First Expansion Step in Conventional Multi-Step TIL Manufacture: pre-REP

A conventional multi-step TIL manufacture process begins with a pre-REP or first expansion. Generally, pre-REP is initiated using a tumor sample that has been fragmented and/or enzymatically digested and to which IL-2 is added for slow cytokine-driven growth of the TILs within the tumor sample. Generally, IL-2 has been the only cytokine, or immunomodulator added to the pre-REP. The pre-REP or first expansion step can take anywhere between 2 weeks and a few months. Pre-REP can begin with obtaining young TILs, which are capable of increased replication cycles upon administration to a subject/patient and as such may provide additional therapeutic benefits over older TILs (i.e., TILs that have further undergone more rounds of replication prior to administration to a subject/patient).

In some embodiments, during pre-REP tumor tissue or cells from tumor tissue are grown in standard lab media (including without limitation RPMI) and treated the with reagents such as irradiated feeder cells and anti-CD3 antibodies to achieve a desired effect, such as increase in the number of TILs and/or an enrichment of the population for cells containing desired cell surface markers or other structural, biochemical or functional features. Pre-REP may utilize lab grade reagents (under the assumption that the lab grade reagents get diluted out during a later REP stage), making it easier to incorporate alternative strategies for improving TIL production. Therefore, in some embodiments, the disclosed TLR agonist and/or peptide or peptidomimetics can be included in the culture medium during the pre-REP stage. The pre-1′P culture can in some embodiments, include 1L-2.

In some cases, after dissection or digestion of tumor fragments, the resulting cells are cultured in media containing IL-2 under conditions that favor the growth of TILs over tumor and other cells. Tumor digests are incubated in 2 mL wells in media comprising inactivated human AB serum with 6000 IU/mL of IL-2. In some examples, 300-6000 IU/mL of IL-2 is added. During pre-REP, this primary cell population is cultured for a period of days to months, resulting in a bulk TIL population, generally about 1×10⁸ bulk TIL cells.

In some cases, during the pre-REP or first expansion step, TIL cultures are initiated by the explant of small (˜2 mm³) tumor fragments or by plating 1×10⁶ viable cells of a single cell suspension of enzymatically digested tumor tissue into 2 ml of complete medium (RPMI1640 based medium supplemented with 10% human serum) containing 6000 IU/ml of IL-2. The cultures are maintained at cell concentrations from 5×10⁵ to 2×10⁶ cells per ml until several million TIL cells are available, usually 2-4 weeks. Multiple independent cultures are screened by cytokine secretion assay for recognition of autologous tumor cells (if available) and HLA-A2+ tumor cell lines. Two to six independent TIL cultures exhibiting the highest cytokine secretion are then further expanded in complete medium with 6000 IU per ml IL-2 until the cell number is over 5×10⁷ cells (this cell number is typically reached 3-6 weeks after tumor excision).

In some cases, the first expansion during pre-REP is performed in a closed system bioreactor, such as G-REX-10 or a G-REX-100.

In the case where genetically modified TILs are to be used in therapy, the first TIL population (also referred to as the bulk TIL population) can be subjected to genetic modifications prior to the second expansion in the REP step.

In conventional processes that incorporate the pre-REP step, the demarcation between the pre-REP and the REP occurs once TIL have undergone expansion in the presence of IL-2 and have either reached an appropriate cell number required to initiate a REP, or have undergone a pre-REP for a predetermined period of time. In various embodiments, a pre-REP may be complete when the number of TIL obtained is 1×10⁶, 10×10⁶, 4×10⁶ or 40×10⁶ cells, depending on the manufacturing protocol used. In another embodiment, a pre-REP may be complete when the duration of culture reached is 3 to 14 days or up to 9 to 14 days from when fragmentation occurs. TIL may then either directly cryopreserved for further use, or transitioned to the REP.

In some cases, the TILs obtained from the pre-REP or first expansion step are stored until phenotyped for selection. In some cases, the TILs obtained from the first expansion are not stored and proceed directly to the second expansion or REP step. In some cases, the TILs obtained from the pre-REP step are not cryopreserved after the first expansion and prior to the second expansion or REP step.

b. Second and Subsequent Expansion Steps in Conventional Multi-Step TIL Manufacture: Rep

In conventional multi-step TIL manufacture, in some cases, the TIL cell population is expanded in number after harvest and initial bulk processing, i.e., pre-REP. This further expansion is referred to as the second expansion, which can include expansion processes generally referred to in the art as a rapid expansion protocol (REP). The second expansion or REP is generally accomplished using a culture media comprising a number of components, including feeder cells, a cytokine source, and an anti-CD3 antibody, in a gas-permeable container. In some cases, the second expansion or REP can be performed using any TIL flasks or containers known by those of skill in the art and can proceed for 7-14 days or longer.

In some cases, the second expansion or REP can be performed in a gas permeable container using methods known in the art. For example, TILs can be rapidly expanded using non-specific T-cell receptor stimulation in the presence of interleukin-2 (IL-2). The non-specific T-cell receptor stimulus can include, for example, an anti-CD3 antibody, such as about 30 ng/ml of OKT3, a mouse monoclonal anti-CD3 antibody (commercially available from Ortho-McNeil, Raritan, N.J. or Miltenyi Biotech, Auburn, Calif.) or UCHT-1 (commercially available from BioLegend, San Diego, Calif., USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2. Other suitable antigens may include, e.g., NY-ESO-1, TRP-1, TRP-2, tyrosinase cancer antigen, MAGE-A3, SSX-2, and VEGFR2, or antigenic portions thereof. TILs may also be rapidly expanded by re-stimulation with the same antigen(s) of the cancer pulsed onto HLA-A2-expressing antigen-presenting cells. Alternatively, the TILs can be further re-stimulated with, e.g., irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In some cases, the second expansion or REP can be conducted in a supplemented cell culture medium comprising IL-2, OKT-3, and antigen-presenting feeder cells. In some cases, the antigen-presenting feeder cells (APCs) are PBMCs (peripheral blood mononuclear cells). In some cases, the ratio of TILs to PBMCs and/or antigen-presenting cells in the rapid expansion and/or the second expansion is 1 to 25 and 1 to 500. In some cases, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 ml media. Media replacement is done (generally ½ or ⅔ media replacement via respiration with fresh media) until the cells are transferred to an alternative growth chamber. Alternative growth chambers include G-REX flasks and gas permeable containers.

In some cases, the second expansion or REP is performed and further comprises a step wherein TILs are selected for superior tumor reactivity. Any selection method known in the art may be used. For example, the methods described in U.S. Patent Application Publication No. 2016/0010058 A1, the disclosure of which is incorporated herein by reference in its entirety, may be used for selection of TILs for superior tumor reactivity. Optionally, a cell viability assay can be performed after the second expansion (including expansions referred to as the REP expansion), using standard assays known in the art. For example, a trypan blue exclusion assay can be done on a sample of the bulk TILs, which selectively labels dead cells and allows a viability assessment. In some cases, TIL samples can be counted and viability determined using a Cellometer K2 automated cell counter (Nexcelom Bioscience, Lawrence, Mass.).

In some cases, further expansion steps can be performed in addition to the second expansion.

c. Feeder Cells

In many cases, the feeder cells used in the conventional multi-step feeder cell-based TIL expansion method are peripheral blood mononuclear cells (PBMCs) obtained from standard whole blood units from healthy blood donors. The PBMCs are obtained using standard methods such as FICOLL-Paque gradient separation. In general, the allogeneic PBMCs are inactivated, either via irradiation or heat treatment, and used in the REP procedures. In some cases, PBMCs are considered replication incompetent and accepted for use in TIL expansion procedures if the total number of viable cells on day 14 is less than the initial viable cell number put into culture on day 0 of the REP and/or day 0 of the second expansion (i.e., the start day of the second expansion).

In some cases, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some cases, the PBMCs are cultured in the presence of 30 ng/ml OKT3 antibody and 3000 IU/ml IL-2.

In some cases, the second expansion or REP procedure requires a ratio of about 2.5×10⁹ feeder cells to between 12.5×10⁶ TILs and 100×10⁶ TILs.

After the second expansion step or REP, cells can be harvested. In some embodiments the TILs are harvested after one, two, three, four or more expansion steps. TILs can be harvested in any appropriate and sterile manner, including for example by centrifugation. Methods for TIL harvesting are well known in the art and any such known methods can be employed with the present process.

### 2. A New One-Step Method for Expanding and Activating TILs

The conventional multi-step feeder cell-dependent expansion and activation process of TILs described above necessitates multiple steps and feeder cells; both requirements render the conventional process time-consuming and expensive. Patients who are in need of immunotherapy using adoptive transfer of tumor infiltrating lymphocytes (TILs) often have very poor prognoses and being treated with a population of expanded and differentiated TILs quickly may constitute the difference between life or death.

In one example, the duration of the expansion is challenging because the patients in need of such therapy are often critically ill and delays can result in death before the treatment is administered. The ability to shorten the duration required for TIL expansion, through the methods described herein, provides a significant advantage over conventional and lengthy processes. In addition, TILs that have been genetically engineered to produce increased effector function as described herein, are advantageous because of their ability to proliferate more rapidly, thus reducing the time for expansion, and to kill tumor cells more effectively.

In addition, the dependence on feeder cells poses challenges for at least a few reasons. First, it is very difficult to obtain a viable feeder cell population, because the cells are collected from 3-5 allogeneic donors. The heterogeneous sourcing of feeder cells renders their use non-standardizable, because each population of donor cells must be qualified individually for its ability to expand TILs. Also, due to the inherent variability in the feeder cells, TIL expansion using the feeder cells becomes less reproducible and predictable. Second, when using feeder cells, TILs can only be engineered, or genetically modified, before or after the REP phase, not during, because the TILs cannot be engineered in the presence of the feeder cells. Third, in TIL manufacturing methods involving a rapid expansion protocol (REP), the REP cannot be shortened because the population of expanded TILs cannot be used until the feeder cells die off. Fourth, the use of feeder cells to stimulate TILs results in the inability to wash out and/or remove the stimulating agents. Therefore, in some cases, the pre-REP and REP steps of the conventional process fail to produce the desired number of TILs. For at least the three reasons delineated above, there is a need, in some embodiments, to eliminate the reliance on feeder cells, which is what has been achieved and disclosed herein. Eliminating feeder cells enables enhanced control over the TIL expansion process. For example, the TIL expansion process can be stopped when the number of TILs required is achieved.

In one aspect of the method disclosed herein, the pre-REP step of the conventional TIL expansion protocol is skipped altogether. Surprisingly, large numbers of TILs can be obtained in 21 days or less during this single expansion step without the use of a pre-REP step, i.e., in a one-step TIL activation and expansion process. In some embodiments, TILs are expanded using a one-step REP-like process depending on feeder cells. In some embodiments, TILs are expanded in a one-step process using particles, such as Dynabeads. In some embodiments, TILs are expanded in a one-step process using tetrameric antibody complexes (TACs), such as from Stemcell. In some embodiments, TILS are expanded in a one-step process using nanomatrices, such as from Miltenyi Biotec (Transact). In some embodiments, TILs are engineered or genetically modified during the one-step TIL expansion process.

In some embodiments, the TILs are from previous failures using the conventional pre-REP described above. In certain embodiments, a pre-REP failure is a failure to expand TILs isolated from a human subject to 4×10⁷ cells in 23 days using the pre-REP protocol. In other embodiments, a pre-REP failure is a failure to expand TILs isolated from a human subject to more than 100× the original number. In other embodiments, a pre-REP failure is a failure to expand TILs isolated from a human subject to 1×10⁶ or 1×10⁷ cells using the pre-REP protocol. In certain embodiments, the methods provided herein are able to rescue pre-REP failures, i.e. expand cells from samples that have experienced a pre-REP failure.

In one aspect of the method disclosed herein, the method of expanding a population of TILs in a disaggregated tumor sample comprises culturing the disaggregated tumor sample in a medium, wherein the TILs are contacted with a T cell receptor (TCR) agonist, a CD28 agonist, and/or a T cell-stimulating cytokine. In some embodiments, the TILs are contacted with a 4-1BB agonist.

In some embodiments, the disaggregated tumor sample comprises tumor fragments that are 0.5 to 4 mm³ in size. In some embodiments, the tumor fragments are 0.5 to 1 mm³ in size. In some embodiments, the tumor fragments are 1 to 1.5 mm³ in size. In some embodiments, the tumor fragments are 1.5 to 2 mm³ in size. In some embodiments, the tumor fragments are 2 to 2.5 mm³ in size. In some embodiments, the tumor fragments are 2.5 to 3 mm³ in size. In some embodiments, the tumor fragments are 3 to 3.5 mm³ in size. In some embodiments, the tumor fragments are 3.5 to 4 mm³ in size. In some embodiments, the disaggregated tumor sample comprises digested tumor fragments.

In some embodiments, the medium is supplemented with the T cell-stimulating cytokine at a time interval ranging from 1-2 days, 2-3 days, 3-4 days, 4-5 days, or 5-6 days. In some embodiments, the time interval is 1 day. In some embodiments, the time interval is 2 days. In some embodiments, the time interval is 3 days. In some embodiments, the time interval is 4 days. In some embodiments, the time interval is 5 days. In some embodiments, the time interval is 6 days.

In some embodiments, the final concentration of the T cell-stimulating cytokine is 10 U/ml to 7,000 U/ml. In some embodiments, the final concentration of the T cell-stimulating cytokine is 100 U/ml to 200 U/ml. In some embodiments, the final concentration of the T cell-stimulating cytokine is 200 U/ml to 300 U/ml. In some embodiments, the final concentration of the T cell-stimulating cytokine is 300 U/ml to 400 U/ml. In some embodiments, the final concentration of the T cell-stimulating cytokine is 400 U/ml to 500 U/ml. In some embodiments, the final concentration of the T cell-stimulating cytokine is 500 U/ml to 600 U/ml. In some embodiments, the final concentration of the T cell-stimulating cytokine is 600 U/ml to 700 U/ml. In some embodiments, the final concentration of the T cell-stimulating cytokine is 700 U/ml to 800 U/ml. In some embodiments, the final concentration of the T cell-stimulating cytokine is 800 U/ml to 900 U/ml. In some embodiments, the final concentration of the T cell-stimulating cytokine is 900 U/ml to 1000 U/ml. In some embodiments, the final concentration of the T cell-stimulating cytokine is 1,000 U/ml to 1,500 U/ml. In some embodiments, the final concentration of the T cell-stimulating cytokine is 1,500 U/ml to 2,000 U/ml. In some embodiments, the final concentration of the T cell-stimulating cytokine is 2,000 U/ml to 2,500 U/ml. In some embodiments, the final concentration of the T cell-stimulating cytokine is 2,500 U/ml to 3,000 U/ml. In some embodiments, the final concentration of the T cell-stimulating cytokine is 3,000 U/ml to 3,500 U/ml. In some embodiments, the final concentration of the T cell-stimulating cytokine is 3,500 U/ml to 4,000 U/ml. In some embodiments, the final concentration of the T cell-stimulating cytokine is 4,000 U/ml to 4,500 U/ml. In some embodiments, the final concentration of the T cell-stimulating cytokine is 4,500 U/ml to 5,000 U/ml. In some embodiments, the final concentration of the T cell-stimulating cytokine is 5,000 U/ml to 5,500 U/ml. In some embodiments, the final concentration of the T cell-stimulating cytokine is 5,500 U/ml to 6,000 U/ml. In some embodiments, the final concentration of the T cell-stimulating cytokine is 6,000 U/ml to 6,500 U/ml. In some embodiments, the final concentration of the T cell-stimulating cytokine is 6,500 U/ml to 7,000 U/ml.

The T-cell stimulating cytokine can be any cytokine effective in stimulating T-cells. In some embodiments, the T cell-stimulating cytokine is IL-2. In some embodiments, the methods disclosed herein further comprise contacting the disaggregated tumor sample and/or population of TILs with the cytokine IL-2. In some embodiments, the TILs are contacted with the cytokine IL-2 every other day. In some embodiments, the TILs are contacted with the cytokine IL-2 in time intervals of 2, 3, 4, 5, or 6 days. In some embodiments, the TILs are contacted with the cytokine IL-2 in a time interval of 2 days. In some embodiments, the TILs are contacted with the cytokine IL-2 in a time interval of 3 days. In some embodiments, the TILs are contacted with the cytokine IL-2 in a time interval of 4 days. In some embodiments, the TILs are contacted with the cytokine IL-2 in a time interval of 5 days. In some embodiments, the TILs are contacted with the cytokine IL-2 in a time interval of 6 days.

In some embodiments, the final concentration of the cytokine IL-2 is 100 U/ml to 7,000 U/ml. In some embodiments, the final concentration of the cytokine IL-2 is 100 U/ml to 200 U/ml. In some embodiments, the final concentration of the cytokine IL-2 is 200 U/ml to 300 U/ml. In some embodiments, the final concentration of the cytokine IL-2 is 300 U/ml to 400 U/ml. In some embodiments, the final concentration of the cytokine IL-2 is 400 U/ml to 500 U/ml. In some embodiments, the final concentration of the cytokine IL-2 is 500 U/ml to 600 U/ml. In some embodiments, the final concentration of the cytokine IL-2 is 600 U/ml to 700 U/ml. In some embodiments, the final concentration of the cytokine IL-2 is 700 U/ml to 800 U/ml. In some embodiments, the final concentration of the cytokine IL-2 is 800 U/ml to 900 U/ml. In some embodiments, the final concentration of the cytokine IL-2 is 900 U/ml to 1000 U/ml. In some embodiments, the final concentration of the cytokine IL-2 is 1,000 U/ml to 1,500 U/ml. In some embodiments, the final concentration of the cytokine IL-2 is 1,500 U/ml to 2,000 U/ml. In some embodiments, the final concentration of the cytokine IL-2 is 2,000 U/ml to 2,500 U/ml. In some embodiments, the final concentration of the cytokine IL-2 is 2,500 U/ml to 3,000 U/ml. In some embodiments, the final concentration of the cytokine IL-2 is 3,000 U/ml to 3,500 U/ml. In some embodiments, the final concentration of the cytokine IL-2 is 3,500 U/ml to 4,000 U/ml. In some embodiments, the final concentration of the cytokine IL-2 is 4,000 U/ml to 4,500 U/ml. In some embodiments, the final concentration of the cytokine IL-2 is 4,500 U/ml to 5,000 U/ml. In some embodiments, the final concentration of the cytokine IL-2 is 5,000 U/ml to 5,500 U/ml. In some embodiments, the final concentration of the cytokine IL-2 is 5,500 U/ml to 6,000 U/ml. In some embodiments, the final concentration of the cytokine IL-2 is 6,000 U/ml to 6,500 U/ml. In some embodiments, the final concentration of the cytokine IL-2 is 6,500 U/ml to 7,000 U/ml.

In some embodiments the components of the medium are maintained. In some embodiments, 30% to 99% of the medium is changed at a time interval ranging from 1-2 days, 2-3 days, 3-4 days, 4-5 days, or 5-6 days. In some embodiments, the time interval is 1 day. In some embodiments, the time interval is 2 days. In some embodiments, the time interval is 3 days. In some embodiments, the time interval is 4 days. In some embodiments, the time interval is 5 days. In some embodiments, the time interval is 6 days.

a. Feeder Cells

In some embodiments, the medium comprises feeder cells. In some embodiments, the feeder cells are peripheral blood mononuclear cells (PBMCs). In some embodiments, the feeder cells are antigen presenting cells (APCs). In some embodiments, the feeder cells express the T cell receptor (TCR) agonist, the CD28 agonist and/or a 4-1BB agonist. In some embodiments, the feeder cells express a 41BB agonist, as described in Bartkowiak and Curran, Front Oncol, 5:117 (2015), incorporated herein by reference in its entirety. In some embodiments, the 4-1BB agonist is 4-1BB ligand. In some embodiments, the T cell receptor (TCR) agonist, CD28 agonist and/or 4-1BB agonist are expressed on the surface of the feeder cells. In some embodiments, the TCR agonist is a CD3 agonist. In some embodiments the CD3 agonist is OKT3 or UCHT. In some embodiments, the CD28 agonist is CD80 or CD86. In some embodiments, the CD28 agonist is CD86. In some embodiments, the CD28 agonist is soluble in the medium. In some embodiments, the feeder cells are APCs. In some embodiments, the APCs are K562 cells. In some embodiments, the APCs are modified to express the proteins described above.

In some embodiments, TILs are genetically modified to reduce expression of one more genes. Additional disclosure regarding these genetic modifications are provided below.

In some embodiments, K562 cells are engineered to express OKT3 “aAPC-OKT3”. In some embodiments, the engineered cells are irradiated prior to use (e.g., with 15,000 rads). In some embodiments, K562 cells are engineered to express OKT3 and CD86 “aAPC-OKT3-CD86”. In some embodiments, the engineered cells are irradiated prior to use (e.g., with 15,000 rads). In some cases, pre-REP failure TILs are expanded with soluble activators or artificial antigen presenting cells (aAPCs).

In some embodiments, the feeder cells are genetically modified to express the T cell-stimulating cytokine. In some embodiments, the T cell-stimulating cytokine that the feeder cells are genetically modified to express is IL-2. In some embodiments, the medium does not comprise feeder cells.

In some embodiments, TILs are expanded using a T cell-stimulating cytokine and feeder cells. In some embodiments, TILs are expanded using a T cell-stimulating cytokine and no feeder cells.

b. Nanomatrices

Nanomatrices smaller than 1 m or 500 nm, having a mobile matrix and having attached thereto stimulatory agent(s) or agonists are able to stimulate T cells. In certain embodiments, the matrix being smaller than 500 nm has no solid phase surface (resulting in a flexible and mobile phase), in contrast to beads or microspheres of the same size. The nanomatrix is like a mesh or net consisting of a mobile polymeric material. In certain embodiments, the polymeric material is dextran. In certain embodiments, the nanomatrix is plastic resulting in the ability to access the cell surface membrane of target cells, e.g., T cells. Therefore, the nanomatrix binds with its agonists attached to the mobile matrix to the respective targets (e.g., receptors) on the cell surface, whereby the flexibility of the matrix allows optimal interaction with the binding partners. To a certain degree the shape of the nanomatrix adapts to the target cell surface thereby extending the contacting surface between nanomatrix and target cell. Due to the size of the matrix of 1 to 500 nm, they are too small to cause perturbance in the cell, i.e., the nanomatrix is biologically inert with regard to alterations of the cell function. Such perturbances triggered by direct cell/bead contact is problematic if beads or microspheres of 1 μm or larger in size are used. In addition, preferentially, the nanomatrix is biodegradable and non-toxic to the cells due to the composition consisting of biodegradable polymeric material, such as a polymer of dextran. In consequence, the nanomatrix is a completely biologically inert entity with regard to alterations of the cell function but biodegradable. Therefore, there is no need to remove the nanomatrix after contacting it with the T cells for stimulation and proliferation. No disturbing effects occur due to the presence of the nanomatrices in an activated T cell composition for subsequent analysis, experiments, and/or clinical applications of these cells.

In addition, due to being soluble or colloidal, the unbound nanomatrices can easily be diluted by repeated washing steps to effective concentrations below the T cell activation threshold after the T-cell stimulation process.

The mobile matrix of the nanomatrix has attached thereto one or more stimulatory agonists which provide activation signal(s) to the T cells, thereby activating and inducing the T cells to proliferate. The agonists are molecules which are capable of binding to a cell surface structure and inducing the polyclonal stimulation of the cells. One example for agents attached to the mobile matrix of the nanomatrix is anti-CD3 monoclonal antibody (mAb) in combination with a co-stimulatory protein such as anti-CD28 mAb.

The feature of the nanomatrix being able to pass sterile filters allows the addition to closed cell culture systems being equipped or equipable with sterile filters, e.g., cell cultivation bags (Miltenyi Biotec, Baxter, CellGenics), G-Rex devices (Wilson Wolf manufacturing), WAVE Bioreactors (GE Healthcare), Quantum Cell Expansion System (Terumo BCT), CliniMACS® Prodigy (Miltenyi Biotec, see Apel et al. 2013, Chemie Ingenieur Technik 85:103-110, incorporated by reference in its entirety herein). The nanomatrix can be added to closed cell culture systems using a syringe to push the nanomatrix through the filter or a pump to pull the nanomatrix through the filter from a bag or a vial (connected to a vented vial adapter).

The contacting can occur e.g., in vitro in any container capable of holding cells, preferably in a sterile environment. Such containers may be e.g., culture flasks, culture bags, bioreactors or any device that can be used to grow cells (e.g., the sample processing system of WO2009072003, i.e., the CliniMACS® Prodigy system, incorporated by reference in its entirety herein).

The nanomatrix used in the present invention can be a nanomatrix wherein at least one first agent and one second agent are attached to the same mobile matrix. Nanomatrices of this kind are contacted with T cells, thereby activating and inducing the T cells to proliferate. The ratio of the first and the second agent attached to the same flexible matrix may be in the range of the ratios of 100:1 to 1:100, preferentially between 10:1 and 1:10, most preferentially between 2:1 and 1:2.

In addition, the nanomatrix of the present invention can be a nanomatrix wherein at least one first agonist and one second agonist are attached to separate mobile matrices. A mixture of these nanomatrices is contacted with T cells, thereby activating and inducing the T cells to proliferate. The ratio and/or concentration of the mobile matrix having attached thereto the first agent and the mobile matrix having attached thereto the second agent may vary to yield optimal stimulation results depending on the kind of T cells used and/or agents used. This facilitates the optimization of the activation conditions for specialized T cell subsets by titrating various concentrations and ratios of the mobile matrix having attached thereto the first agent and the mobile matrix having attached thereto the second agent.

Nanomatrices can be prepared by various methods known in the art, including solvent evaporation, phase separation, spray-drying, or solvent extraction at low temperature. The process selected should be simple, reproducible and scalable. The resulting nanomatrices should be free-flowing and not aggregate in order to produce a uniform syringeable suspension. The nanomatrix should also be sterile. This can be ensured by e.g., filtration, a terminal sterilization step and/or through aseptic processing. A preparation of nanomatrices is described in Example 1.

The terms “matrix of mobile polymer chains” and “mobile matrix” as used herein have an interchangeable meaning. The term “mobile” refers to the common and well described feature of organic biopolymers such as dextran or others on nanoparticles (see Bertholon et al. Langmuir 2006, pp 45485-5490, incorporated by reference herein in its entirety). These polymers include mobile (motile), preferentially highly mobile (motile) chains, so the matrix is characterized by the absence of a solid surface as the attachment point for the stimulating agents such as antibodies, and which is in strong contrast to currently used beads or microspheres which regularly have an inflexible, stiff surface. As a result, the nanomatrix comprising a matrix of mobile polymer chains is flexible and adjustable to the form of the surface of the cells. In addition, as a result, the nanomatrix is a nanomatrix wherein the majority (i.e., more than 50%), preferentially more than 80% and more preferentially more than 90% and most preferentially more than 99% of the total volume of the nanomatrix in aqueous solution consists of mobile polymer chains.

The contact between nanomatrix which has coupled thereto one or more stimulatory agents and the cells to be stimulated benefit from the fact that the nanomatrix does not have a fixed, stiff or rigid surface, allowing the nanomatrix to access the cell surface. In certain embodiments, the nanomatrix is composed of hydrophilic polymer chains, which obtain maximal mobility in aqueous solution due to hydration of the chains. The mobile matrix is the only or at least main component of the nanomatrix regardless the agents which are attached thereto.

An agonist may be attached or coupled to the mobile matrix by a variety of methods known and available in the art. The attachment may be covalent or noncovalent, electrostatic, or hydrophobic and may be accomplished by a variety of attachment means, including, for example, chemical, mechanical, enzymatic, or other means whereby an agent is capable of stimulating the cells. For example, the antibody to a cell surface structure first may be attached to the matrix, or avidin or streptavidin may be attached to the matrix for binding to a biotinylated agent. The antibody to the cell surface structure may be attached to the matrix directly or indirectly, e.g., via an anti-isotype antibody. Another example includes using protein A or protein G, or other non-specific antibody binding molecules, attached to matrices to bind an antibody. Alternatively, the agent may be attached to the matrix by chemical means, such as cross-linking to the matrix.

The phrase “biologically inert” as used herein refers to the properties of the nanomatrix, that it is non-toxic to living cells and does not induce strong alterations of the cell function via physical interaction with the cell surface, due to its small size, except the specific ligand/receptor triggering function of the attached ligands or antibodies. The nanomatrices, in addition, may be biodegradable, e.g., degraded by enzymatic activity or cleared by phagocytic cells. The biodegradable material can be derived from natural or synthetic materials that degrade in biological fluids, e.g., cell culture media and blood. The degradation may occur using enzymatic means or may occur without enzymatic means. The biodegradable material degrades within days, weeks or a few months, which may depend on the environmental conditions it is exposed to. The biodegradable material should be non-toxic and non-antigenic for living cells and in humans. The degradation products must produce non-toxic by-products. An important aspect in the context of being biologically inert is the fact that the nanomatrix does not induce strong alteration in structure, function, activity status or viability of labelled cells, i.e., it does not cause perturbance of the cells and does not interfere with subsequent experiments and therapeutic applications of the stimulated cells. The mechanical or chemical irritation of the cell is decreased due to the properties of the nanomatrix of being very small, i.e., nano-scale range, and having a mobile matrix which rather snuggles to the cell surface than altering the shape of the cell surface or exerting strong shearing force to the cells, e.g., resulting in membrane rupture.

In some embodiments, the TCR agonist and/or the CD28 agonist are linked to a nanomatrix comprising a colloidal suspension of matrices of polymer chains, wherein each nanomatrix is 1 to 500 nm in length in its largest dimension. In some embodiments, the nanomatrix is 1 to 50 nm in length in its largest dimension. In some embodiments, the nanomatrix is 50 to 100 nm in length in its largest dimension. In some embodiments, the nanomatrix is 100 to 150 nm in length in its largest dimension. In some embodiments, the nanomatrix is 150 to 200 nm in length in its largest dimension. In some embodiments, the nanomatrix is 200 to 250 nm in length in its largest dimension. In some embodiments, the nanomatrix is 250 to 300 nm in length in its largest dimension. In some embodiments, the nanomatrix is 300 to 350 nm in length in its largest dimension. In some embodiments, the nanomatrix is 350 to 400 nm in length in its largest dimension. In some embodiments, the nanomatrix is 400 to 450 nm in length in its largest dimension. In some embodiments, the nanomatrix is 450 to 500 nm in length in its largest dimension.

In some embodiments, the TCR agonist and the CD28 agonist are attached to the same polymer chains. In some embodiments, the TCR agonist and the CD28 agonist are attached to different polymer chains. In some embodiments, the TCR agonist, or fragment thereof, is attached to the nanomatrix at 25 μg per mg of nanomatrix. In some embodiments, the TCR agonist, or fragment thereof, is attached to the nanomatrix at about 5 μg to about 10 μg per mg of nanomatrix. In some embodiments, the TCR agonist, or fragment thereof, is attached to the nanomatrix at about 10 μg to about 15 μg per mg of nanomatrix. In some embodiments, the TCR agonist, or fragment thereof, is attached to the nanomatrix at about 15 μg to about 20 μg per mg of nanomatrix. In some embodiments, the TCR agonist, or fragment thereof, is attached to the nanomatrix at about 20 μg to about 25 μg per mg of nanomatrix. In some embodiments, the TCR agonist, or fragment thereof, is attached to the nanomatrix at about 25 μg to about 30 μg per mg of nanomatrix. In some embodiments, the TCR agonist, or fragment thereof, is attached to the nanomatrix at about 30 μg to about 35 μg per mg of nanomatrix. In some embodiments, the TCR agonist, or fragment thereof, is attached to the nanomatrix at about 35 μg to about 40 μg per mg of nanomatrix. In some embodiments, the TCR agonist, or fragment thereof, is attached to the nanomatrix at about 40 μg to about 45 μg per mg of nanomatrix. In some embodiments, the TCR agonist, or fragment thereof, is attached to the nanomatrix at about 45 μg to about 50 μg per mg of nanomatrix. In some embodiments, the TCR agonist is a CD3 agonist.

In some embodiments, the CD28 agonist, or fragment thereof, is attached to the nanomatrix at 25 μg per mg of nanomatrix. In some embodiments, the CD28 agonist, or fragment thereof, is attached to the nanomatrix at about 5 μg to about 10 μg per mg of nanomatrix. In some embodiments, the CD28 agonist, or fragment thereof, is attached to the nanomatrix at about 10 μg to about 15 μg per mg of nanomatrix. In some embodiments, the CD28 agonist, or fragment thereof, is attached to the nanomatrix at about 15 μg to about 20 μg per mg of nanomatrix. In some embodiments, the CD28 agonist, or fragment thereof, is attached to the nanomatrix at about 20 μg to about 25 μg per mg of nanomatrix. In some embodiments, the CD28 agonist, or fragment thereof, is attached to the nanomatrix at about 25 pg to about 30 μg per mg of nanomatrix. In some embodiments, the CD28 agonist, or fragment thereof, is attached to the nanomatrix at about 30 μg to about 35 μg per mg of nanomatrix. In some embodiments, the CD28 agonist, or fragment thereof, is attached to the nanomatrix at about 35 μg to about 40 μg per mg of nanomatrix. In some embodiments, the CD28 agonist, or fragment thereof, is attached to the nanomatrix at about 40 μg to about 45 μg per mg of nanomatrix. In some embodiments, the CD28 agonist, or fragment thereof, is attached to the nanomatrix at about 45 μg to about 50 μg per mg of nanomatrix.

In some embodiments, the nanomatrix further comprises magnetic, paramagnetic or superparamagnetic nanocrystals embedded among or within the matrices of polymer chains. In some embodiments, the matrix of polymer chains comprises a polymer of dextran. In some embodiments, the polymer chains are colloidal polymer chains.

In some embodiments, the ratio of volume of nanomatrix to volume of TILs in the disaggregated tumor sample is greater than or equal to 1:5. In some embodiments, the ratio of volume of nanomatrix to volume of TILs is greater than or equal to 1:10. In some embodiments, the ratio of volume of nanomatrix to volume of TILs is greater than or equal to 1:25. In some embodiments, the ratio of volume of nanomatrix to volume of TILs is greater than or equal to 1:50. In some embodiments, the ratio of volume of nanomatrix to volume of TILs is greater than or equal to 1:100. In some embodiments, the ratio of volume of nanomatrix to volume of TILs is greater than or equal to 1:200. In some embodiments, the ratio of volume of nanomatrix to volume of TILs is greater than or equal to 1:300. In some embodiments, the ratio of volume of nanomatrix to volume of TILs is greater than or equal to 1:400. In some embodiments, the ratio of volume of nanomatrix to volume of TILs is greater than or equal to 1:500. In some embodiments, the ratio of volume of nanomatrix to volume of TILs is greater than or equal to 1:600. In some embodiments, the ratio of volume of nanomatrix to volume of TILs is greater than or equal to 1:700. In some embodiments, the ratio of volume of nanomatrix to volume of TILs is greater than or equal to 1:800. In some embodiments, the ratio of volume of nanomatrix to volume of TILs is greater than or equal to 1:900. In some embodiments, the ratio of volume of nanomatrix to volume of TILs is greater than or equal to 1:1,000.

In some embodiments, the ratio of number of matrices to TILs in the disaggregated tumor sample is greater than or equal to 1:500. In some embodiments, the ratio of number of matrices to TILs is 1:500 to 1:750. In some embodiments, the ratio of number of matrices to TILs is 1:750 to 1:1,000. In some embodiments, the ratio of number of matrices to TILs is 1:1,000 to 1:1,250. In some embodiments, the ratio of number of matrices to TILs is 1:1,250 to 1:1,500. In some embodiments, the ratio of number of matrices to TILs is 1:1,500 to 1:1,750. In some embodiments, the ratio of number of matrices to TILs is 1:1,750 to 1:2,000. In some embodiments, the ratio of number of matrices to TILs is 1:2,000 to 1:2,250. In some embodiments, the ratio of number of matrices to TILs is 1:2,250 to 1:2,500. In some embodiments, the ratio of number of matrices to TILs is 1:2,500 to 1:2,750. In some embodiments, the ratio of number of matrices to TILs is 1:2,750 to 1:3,000. In some embodiments, the ratio of number of matrices to TILs is 1:3,000 to 1:3,500. In some embodiments, the ratio of number of matrices to TILs is 1:3,500 to 1:4,000. In some embodiments, the ratio of number of matrices to TILs is 1:4,000 to 1:5,000.

In some embodiments, the agonists are recombinant agonists. In some embodiments, the agonists are antibodies. In some embodiments, the antibodies are humanized antibodies. In some embodiments, the CD3 agonist is an OKT3 antibody or an UCHT1 antibody.

In another aspect of the method disclosed herein, the method for expanding a population of TILs comprises contacting the population of TILs with a nanomatrix comprising a colloidal suspension of matrices of polymer chains, wherein the matrices are attached to CD3 agonists and CD28 agonists, wherein the nanomatrix provides activation signals to the population of TILs, thereby activating and inducing the population of TILs to proliferate, wherein each matrix is 1 to 500 nm in length in its largest dimension, and wherein the method does not comprise the use of feeder cells during expansion of the population of TILs.

In some embodiments, the population of TILs contacted with the nanomatrix further comprises tumor cells. In some embodiments, the population of TILs is isolated from a subject and contacted with the nanomatrix without an additional expansion process of the population of TILs prior to contacting the population of TILs with the nanomatrix.

In some embodiments, the CD3 agonists and the CD28 agonists are attached to the same polymer chains. In some embodiments, the CD3 agonists and the CD28 agonists are attached to different polymer chains. In some embodiments, the CD3 agonists, or fragments thereof, are attached to the nanomatrix at 25 μg per mg of nanomatrix. In some embodiments, the CD3 agonists, or fragments thereof, are attached to the nanomatrix at about 5 μg to about 10 μg per mg of nanomatrix. In some embodiments, the CD3 agonists, or fragments thereof, are attached to the nanomatrix at about 10 μg to about 15 μg per mg of nanomatrix. In some embodiments, the CD3 agonists, or fragments thereof, are attached to the nanomatrix at about 15 μg to about 20 μg per mg of nanomatrix. In some embodiments, the CD3 agonists, or fragments thereof, are attached to the nanomatrix at about 20 μg to about 25 μg per mg of nanomatrix. In some embodiments, the CD3 agonists, or fragments thereof, are attached to the nanomatrix at about 25 μg to about 30 μg per mg of nanomatrix. In some embodiments, the CD3 agonists, or fragments thereof, are attached to the nanomatrix at about 30 μg to about 35 μg per mg of nanomatrix. In some embodiments, the CD3 agonists, or fragments thereof, are attached to the nanomatrix at about 35 μg to about 40 μg per mg of nanomatrix. In some embodiments, the CD3 agonists, or fragments thereof, are attached to the nanomatrix at about 40 μg to about 45 μg per mg of nanomatrix. In some embodiments, the CD3 agonists, or fragments thereof, are attached to the nanomatrix at about 45 μg to about 50 μg per mg of nanomatrix.

In some embodiments, the CD28 agonists, or fragments thereof, are attached to the nanomatrix at 25 μg per mg of nanomatrix. In some embodiments, the CD28 agonists, or fragments thereof, are attached to the nanomatrix at about 5 μg to about 10 μg per mg of nanomatrix. In some embodiments, the CD28 agonists, or fragments thereof, are attached to the nanomatrix at about 10 μg to about 15 μg per mg of nanomatrix. In some embodiments, the CD28 agonists, or fragments thereof, are attached to the nanomatrix at about 15 μg to about 20 μg per mg of nanomatrix. In some embodiments, the CD28 agonists, or fragments thereof, are attached to the nanomatrix at about 20 μg to about 25 μg per mg of nanomatrix. In some embodiments, the CD28 agonists, or fragments thereof, are attached to the nanomatrix at about 25 μg to about 30 μg per mg of nanomatrix. In some embodiments, the CD28 agonists, or fragments thereof, are attached to the nanomatrix at about 30 μg to about 35 μg per mg of nanomatrix. In some embodiments, the CD28 agonists, or fragments thereof, are attached to the nanomatrix at about 35 μg to about 40 μg per mg of nanomatrix. In some embodiments, the CD28 agonists, or fragments thereof, are attached to the nanomatrix at about 40 μg to about 45 μg per mg of nanomatrix. In some embodiments, the CD28 agonists, or fragments thereof, are attached to the nanomatrix at about 45 μg to about 50 μg per mg of nanomatrix.

In some embodiments, the nanomatrix further comprises magnetic, paramagnetic or superparamagnetic nanocrystals embedded among or within the matrices of polymer chains. In some embodiments, the matrix of polymer chains comprises a polymer of dextran. In some embodiments, the polymer chains are colloidal polymer chains.

In some embodiments, the ratio of volume of nanomatrix to volume of TILs is greater than or equal to 1:5. In some embodiments, the ratio of volume of nanomatrix to volume of TILs is greater than or equal to 1:10. In some embodiments, the ratio of volume of nanomatrix to volume of TILs is greater than or equal to 1:25. In some embodiments, the ratio of volume of nanomatrix to volume of TILs is greater than or equal to 1:50. In some embodiments, the ratio of volume of nanomatrix to volume of TILs is greater than or equal to 1:100. In some embodiments, the ratio of volume of nanomatrix to volume of TILs is greater than or equal to 1:200. In some embodiments, the ratio of volume of nanomatrix to volume of TILs is greater than or equal to 1:300. In some embodiments, the ratio of volume of nanomatrix to volume of TILs is greater than or equal to 1:400. In some embodiments, the ratio of volume of nanomatrix to volume of TILs is greater than or equal to 1:500. In some embodiments, the ratio of volume of nanomatrix to volume of TILs is greater than or equal to 1:600. In some embodiments, the ratio of volume of nanomatrix to volume of TILs is greater than or equal to 1:700. In some embodiments, the ratio of volume of nanomatrix to volume of TILs is greater than or equal to 1:800. In some embodiments, the ratio of volume of nanomatrix to volume of TILs is greater than or equal to 1:900. In some embodiments, the ratio of volume of nanomatrix to volume of TILs is greater than or equal to 1:1,000.

In some embodiments, the ratio of number of matrices to TILs is greater than or equal to 1:500. In some embodiments, the ratio of number of matrices to TILs is 1:500 to 1:750. In some embodiments, the ratio of number of matrices to TILs is 1:750 to 1:1,000. In some embodiments, the ratio of number of matrices to TILs is 1:1,000 to 1:1,250. In some embodiments, the ratio of number of matrices to TILs is 1:1,250 to 1:1,500. In some embodiments, the ratio of number of matrices to TILs is 1:1,500 to 1:1,750. In some embodiments, the ratio of number of matrices to TILs is 1:1,750 to 1:2,000. In some embodiments, the ratio of number of matrices to TILs is 1:2,000 to 1:2,250. In some embodiments, the ratio of number of matrices to TILs is 1:2,250 to 1:2,500. In some embodiments, the ratio of number of matrices to TILs is 1:2,500 to 1:2,750. In some embodiments, the ratio of number of matrices to TILs is 1:2,750 to 1:3,000. In some embodiments, the ratio of number of matrices to TILs is 1:3,000 to 1:3,500. In some embodiments, the ratio of number of matrices to TILs is 1:3,500 to 1:4,000. In some embodiments, the ratio of number of matrices to TILs is 1:4,000 to 1:5,000.

In some embodiments, the agonists are recombinant agonists. In some embodiments, the agonists are antibodies. In some embodiments, the antibodies are humanized antibodies. In some embodiments, the CD3 agonist is an OKT3 antibody or an UCHT1 antibody.

c. Soluble Monospecific Complexes

In another aspect of the method disclosed herein, the method for expanding a population of TILs comprises contacting the population of TILs with a composition comprising a first, a second, and a third soluble monospecific complex, wherein each soluble monospecific complex comprises two antibodies or fragments thereof linked together, wherein each antibody or fragments thereof of each soluble monospecific complex specifically binds to the same antigen on the population of TILs, wherein the first soluble monospecific complex comprises an anti-CD3 antibody, wherein the second soluble monospecific complex comprises an anti-CD28 antibody, and wherein the third soluble monospecific complex comprises an anti-CD2 antibody, and the method does not comprise the use of feeder cells during expansion of the population of TILs.

In some embodiments, the TCR agonist comprises a soluble monospecific complex comprising two anti-CD3 antibodies linked together. In some embodiments, the CD28 agonist comprises a soluble monospecific complex comprising two anti-CD28 antibodies linked together.

In some embodiments, the medium comprises a CD2 agonist. In some embodiments, the CD2 agonist comprises a soluble monospecific complex comprising two anti-CD2 antibodies linked together.

In some embodiments, the soluble monospecific complexes are at a concentration of 0.2-25 μl/ml. In some embodiments, the soluble monospecific complexes are at a concentration of 0.2-1 μl/ml. In some embodiments, the soluble monospecific complexes are at a concentration of 1-2 μl/ml. In some embodiments, the soluble monospecific complexes are at a concentration of 2-5 μl/ml. In some embodiments, the soluble monospecific complexes are at a concentration of 5-10 μl/ml. In some embodiments, the soluble monospecific complexes are at a concentration of 10⁻¹⁵ μl/ml. In some embodiments, the soluble monospecific complexes are at a concentration of 15-20 μl/ml. In some embodiments, the soluble monospecific complexes are at a concentration of 20-25 μl/ml. In some embodiments, the soluble monospecific complexes are tetrameric antibody complexes (TACs). In some embodiments, each TAC comprises two antibodies from a first animal species bound by two antibody molecules from a second species that specifically bind to the Fc portion of the antibodies from the first animal species. In some embodiments, the anti-CD3 antibody is an OKT3 antibody or an UCHT1 antibody. In some embodiments, soluble monospecific complexes are particularly effective increasing the central memory T cell phenotype.

d. TILs Expansion

In some embodiments, the TILs are expanded for up to a total of 9, 10, 11, 12, 13, 14, 15, 16, 17, 18, 19, 20, 21, 22, 23, 24 or 25 days from the initial tumor fragmentation or disaggregation. In some embodiments, the TILs are expanded for a total of 9-25 days, 9-21 days, or 9-14 days. In some embodiments, the TILs are expanded for up to a total of 9 days. In some embodiments, the TILs are expanded for up to a total of 10 days. In some embodiments, the TILs are expanded for up to a total of 11 days. In some embodiments, the TILs are expanded for up to a total of 12 days. In some embodiments, the TILs are expanded for up to a total of 13 days. In some embodiments, the TILs are expanded for up to a total of 14 days. In some embodiments, the TILs are expanded for up to a total of 15 days. In some embodiments, the TILs are expanded for up to a total of 16 days. In some embodiments, the TILs are expanded for up to a total of 17 days. In some embodiments, the TILs are expanded for up to a total of 18 days. In some embodiments, the TILs are expanded for up to a total of 19 days. In some embodiments, the TILs are expanded for up to a total of 20 days. In some embodiments, the TILs are expanded for up to a total of 21 days. In some embodiments, the TILs are expanded for up to a total of 22 days. In some embodiments, the TILs are expanded for up to a total of 23 days. In some embodiments, the TILs are expanded for up to a total of 24 days. In some embodiments, the TILs are expanded for up to a total of 25 days.

In some embodiments, the population of TILs is expanded 500 to 500,000-fold. In some embodiments, the population of TILs is expanded 500 to 1,000-fold. In some embodiments, the population of TILs is expanded 1,000 to 2,500-fold. In some embodiments, the population of TILs is expanded 2,500 to 5,000-fold. In some embodiments, the population of TILs is expanded 5,000 to 10,000-fold. In some embodiments, the population of TILs is expanded 10,000 to 20,000-fold. In some embodiments, the population of TILs is expanded 20,000 to 30,000-fold. In some embodiments, the population of TILs is expanded 30,000 to 40,000-fold. In some embodiments, the population of TILs is expanded 40,000 to 50,000-fold. In some embodiments, the population of TILs is expanded 50,000 to 100,000-fold. In some embodiments, the population of TILs is expanded 100,000 to 150,000-fold. In some embodiments, the population of TILs is expanded 150,000 to 200,000-fold. In some embodiments, the population of TILs is expanded 200,000 to 250,000-fold. In some embodiments, the population of TILs is expanded 250,000 to 300,000-fold. In some embodiments, the population of TILs is expanded 300,000 to 350,000-fold. In some embodiments, the population of TILs is expanded 350,000 to 400,000-fold. In some embodiments, the population of TILs is expanded 400,000 to 450,000-fold. In some embodiments, the population of TILs is expanded 450,000 to 500,000-fold.

In some embodiments, the population of TILs is expanded from an initial population of TILs of between 100 and 100,000 TILs. In some embodiments, the population of TILs is expanded from an initial population of TILs of between 100 and 1,000 TILs. In some embodiments, the population of TILs is expanded from an initial population of TILs of between 1,000 and 2,500 TILs. In some embodiments, the population of TILs is expanded from an initial population of TILs of between 2,500 and 5,000 TILs. In some embodiments, the population of TILs is expanded from an initial population of TILs of between 5,000 and 7,500 TILs. In some embodiments, the population of TILs is expanded from an initial population of TILs of between 7,500 and 10,000 TILs. In some embodiments, the population of TILs is expanded from an initial population of TILs of between 10,000 and 20,000 TILs. In some embodiments, the population of TILs is expanded from an initial population of TILs of between 20,000 and 30,000 TILs. In some embodiments, the population of TILs is expanded from an initial population of TILs of between 30,000 and 40,000 TILs. In some embodiments, the population of TILs is expanded from an initial population of TILs of between 40,000 and 50,000 TILs. In some embodiments, the population of TILs is expanded from an initial population of TILs of between 50,000 and 60,000 TILs. In some embodiments, the population of TILs is expanded from an initial population of TILs of between 60,000 and 70,000 TILs. In some embodiments, the population of TILs is expanded from an initial population of TILs of between 70,000 and 80,000 TILs. In some embodiments, the population of TILs is expanded from an initial population of TILs of between 80,000 and 90,000 TILs. In some embodiments, the population of TILs is expanded from an initial population of TILs of between 90,000 and 100,000 TILs.

In some embodiments, the population of TILs is expanded at least 150-fold at day 10 of the expansion. In some embodiments, the population of TILs is expanded at least 500-fold at day 10 of the expansion. In some embodiments, the population of TILs is expanded at least 750-fold at day 10 of the expansion. In some embodiments, the population of TILs is expanded at least 1000-fold at day 10 of the expansion. In some embodiments, the population of TILs is expanded at least 1500-fold at day 10 of the expansion. In some embodiments, the population of TILs is expanded at least 2000-fold at day 10 of the expansion. In some embodiments, the population of TILs is expanded at least 2500-fold at day 10 of the expansion. In some embodiments, the population of TILs is expanded at least 3000-fold at day 10 of the expansion. In some embodiments, the population of TILs is expanded at least 4000-fold at day 10 of the expansion. In some embodiments, the population of TILs is expanded at least 5000-fold at day 10 of the expansion. In some embodiments, the population of TILs is expanded at least 6000-fold at day 10 of the expansion. In some embodiments, the population of TILs is expanded at least 7000-fold at day 10 of the expansion. In some embodiments, the population of TILs is expanded at least 8000-fold at day 10 of the expansion. In some embodiments, the population of TILs is expanded at least 9000-fold at day 10 of the expansion. In some embodiments, the population of TILs is expanded at least 10,000-fold at day 10 of the expansion. In some embodiments, these fold expansions on day 10 occurred with TILs from pre-REP failures.

In some embodiments, the population of TILs is expanded at least 1,500-fold at day 14 of the expansion. In some embodiments, the population of TILs is expanded at least 5,000-fold at day 14 of the expansion. In some embodiments, the population of TILs is expanded at least 7,500-fold at day 14 of the expansion. In some embodiments, the population of TILs is expanded at least 10,000-fold at day 14 of the expansion. In some embodiments, the population of TILs is expanded at least 15,000-fold at day 14 of the expansion. In some embodiments, the population of TILs is expanded at least 20,000-fold at day 14 of the expansion. In some embodiments, the population of TILs is expanded at least 25,000-fold at day 14 of the expansion. In some embodiments, the population of TILs is expanded at least 30,000-fold at day 14 of the expansion. In some embodiments, the population of TILs is expanded at least 40,000-fold at day 14 of the expansion. In some embodiments, the population of TILs is expanded at least 50,000-fold at day 14 of the expansion. In some embodiments, the population of TILs is expanded at least 60,000-fold at day 14 of the expansion. In some embodiments, the population of TILs is expanded at least 70,000-fold at day 14 of the expansion. In some embodiments, the population of TILs is expanded at least 80,000-fold at day 14 of the expansion. In some embodiments, the population of TILs is expanded at least 90,000-fold at day 14 of the expansion. In some embodiments, the population of TILs is expanded at least 100,000-fold at day 14 of the expansion. In some embodiments, the population of TILs is expanded at least 110,000-fold at day 14 of the expansion. In some embodiments, the population of TILs is expanded at least 120,000-fold at day 14 of the expansion. In some embodiments, the population of TILs is expanded at least 130,000-fold at day 14 of the expansion. In some embodiments, the population of TILs is expanded at least 140,000-fold at day 14 of the expansion. In some embodiments, these fold expansions on day 14 occurred with TILs from pre-REP failures.

In some embodiments, the population of TILs is expanded at most 150,000-fold at day 14 of the expansion. In some embodiments, the population of TILs is expanded at most 5,000-fold at day 14 of the expansion. In some embodiments, the population of TILs is expanded at most 7,500-fold at day 14 of the expansion. In some embodiments, the population of TILs is expanded at most 10,000-fold at day 14 of the expansion. In some embodiments, the population of TILs is expanded at most 15,000-fold at day 14 of the expansion. In some embodiments, the population of TILs is expanded at most 20,000-fold at day 14 of the expansion. In some embodiments, the population of TILs is expanded at most 25,000-fold at day 14 of the expansion. In some embodiments, the population of TILs is expanded at most 30,000-fold at day 14 of the expansion. In some embodiments, the population of TILs is expanded at most 40,000-fold at day 14 of the expansion. In some embodiments, the population of TILs is expanded at most 50,000-fold at day 14 of the expansion. In some embodiments, the population of TILs is expanded at most 60,000-fold at day 14 of the expansion. In some embodiments, the population of TILs is expanded at most 70,000-fold at day 14 of the expansion. In some embodiments, the population of TILs is expanded at most 80,000-fold at day 14 of the expansion. In some embodiments, the population of TILs is expanded at most 90,000-fold at day 14 of the expansion. In some embodiments, the population of TILs is expanded at most 100,000-fold at day 14 of the expansion. In some embodiments, the population of TILs is expanded at most 110,000-fold at day 14 of the expansion. In some embodiments, the population of TILs is expanded at most 120,000-fold at day 14 of the expansion. In some embodiments, the population of TILs is expanded at most 130,000-fold at day 14 of the expansion. In some embodiments, the population of TILs is expanded at most 140,000-fold at day 14 of the expansion. In some embodiments, these fold expansions on day 14 occurred with TILs from pre-REP failures.

In some embodiments, the population of TILs is expanded at least 15,000-fold at day 21 of the expansion. In some embodiments, the population of TILs is expanded at least 20,000-fold at day 21 of the expansion. In some embodiments, the population of TILs is expanded at least 25,000-fold at day 21 of the expansion. In some embodiments, the population of TILs is expanded at least 30,000-fold at day 21 of the expansion. In some embodiments, the population of TILs is expanded at least 40,000-fold at day 21 of the expansion. In some embodiments, the population of TILs is expanded at least 50,000-fold at day 21 of the expansion. In some embodiments, the population of TILs is expanded at least 60,000-fold at day 21 of the expansion. In some embodiments, the population of TILs is expanded at least 70,000-fold at day 21 of the expansion. In some embodiments, the population of TILs is expanded at least 80,000-fold at day 21 of the expansion. In some embodiments, the population of TILs is expanded at least 90,000-fold at day 21 of the expansion. In some embodiments, the population of TILs is expanded at least 100,000-fold at day 21 of the expansion. In some embodiments, the population of TILs is expanded at least 110,000-fold at day 21 of the expansion. In some embodiments, the population of TILs is expanded at least 120,000-fold at day 21 of the expansion. In some embodiments, the population of TILs is expanded at least 130,000-fold at day 21 of the expansion. In some embodiments, the population of TILs is expanded at least 140,000-fold at day 21 of the expansion. In some embodiments, the population of TILs is expanded at least 150,000-fold at day 21 of the expansion. In some embodiments, the population of TILs is expanded at least 200,000-fold at day 21 of the expansion. In some embodiments, the population of TILs is expanded at least 300,000-fold at day 21 of the expansion. In some embodiments, the population of TILs is expanded at least 400,000-fold at day 21 of the expansion. In some embodiments, these fold expansions on day 21 occurred with TILs from pre-REP failures.

In some embodiments, the population of TILs is expanded at most 500,000-fold at day 21 of the expansion. In some embodiments, the population of TILs is expanded at most 20,000-fold at day 21 of the expansion. In some embodiments, the population of TILs is expanded at most 25,000-fold at day 21 of the expansion. In some embodiments, the population of TILs is expanded at most 30,000-fold at day 21 of the expansion. In some embodiments, the population of TILs is expanded at most 40,000-fold at day 21 of the expansion. In some embodiments, the population of TILs is expanded at most 50,000-fold at day 21 of the expansion. In some embodiments, the population of TILs is expanded at most 60,000-fold at day 21 of the expansion. In some embodiments, the population of TILs is expanded at most 70,000-fold at day 21 of the expansion. In some embodiments, the population of TILs is expanded at most 80,000-fold at day 21 of the expansion. In some embodiments, the population of TILs is expanded at most 90,000-fold at day 21 of the expansion. In some embodiments, the population of TILs is expanded at most 100,000-fold at day 21 of the expansion. In some embodiments, the population of TILs is expanded at most 110,000-fold at day 21 of the expansion. In some embodiments, the population of TILs is expanded at most 120,000-fold at day 21 of the expansion. In some embodiments, the population of TILs is expanded at most 130,000-fold at day 21 of the expansion. In some embodiments, the population of TILs is expanded at most 140,000-fold at day 21 of the expansion. In some embodiments, the population of TILs is expanded at most 150,000-fold at day 21 of the expansion. In some embodiments, the population of TILs is expanded at most 200,000-fold at day 21 of the expansion. In some embodiments, the population of TILs is expanded at most 300,000-fold at day 21 of the expansion. In some embodiments, the population of TILs is expanded at most 400,000-fold at day 21 of the expansion. In some embodiments, these fold expansions on day 21 occurred with TILs from pre-REP failures.

In some embodiments, members of the population of TILs are genetically modified. In some embodiments, the population of TILs is genetically modified using an RNA-guided nuclease. In some embodiments, the population of TILs is genetically modified using Cas9 and at least one guide RNA. In some embodiments, members of the population of TILs are epigenetically modified.

In some embodiments, the population of TILs is expanded to produce an expanded population of TILs, wherein at least 2% of the expanded population have a central memory T cell phenotype. In some embodiments, the population of TILs is expanded to produce an expanded population of TILs, wherein at least 3% of the expanded population have a central memory T cell phenotype. In some embodiments, the population of TILs is expanded to produce an expanded population of TILs, wherein at least 4% of the expanded population have a central memory T cell phenotype. In some embodiments, the population of TILs is expanded to produce an expanded population of TILs, wherein at least 5% of the expanded population have a central memory T cell phenotype. In some embodiments, the population of TILs is expanded to produce an expanded population of TILs, wherein at least 6% of the expanded population have a central memory T cell phenotype. In some embodiments, the population of TILs is expanded to produce an expanded population of TILs, wherein at least 7% of the expanded population have a central memory T cell phenotype. In some embodiments, the population of TILs is expanded to produce an expanded population of TILs, wherein at least 8% of the expanded population have a central memory T cell phenotype. In some embodiments, the population of TILs is expanded to produce an expanded population of TILs, wherein at least 9% of the expanded population have a central memory T cell phenotype. In some embodiments, the population of TILs is expanded to produce an expanded population of TILs, wherein at least 10% of the expanded population have a central memory T cell phenotype. In some embodiments, the population of TILs is expanded to produce an expanded population of TILs, wherein at least 11% of the expanded population have a central memory T cell phenotype. In some embodiments, the population of TILs is expanded to produce an expanded population of TILs, wherein at least 12% of the expanded population have a central memory T cell phenotype. In some embodiments, the population of TILs is expanded to produce an expanded population of TILs, wherein at least 13% of the expanded population have a central memory T cell phenotype. In some embodiments, the population of TILs is expanded to produce an expanded population of TILs, wherein at least 14% of the expanded population have a central memory T cell phenotype. In some embodiments, the population of TILs is expanded to produce an expanded population of TILs, wherein at least 15% of the expanded population have a central memory T cell phenotype.

In some embodiments, the population of TILs is expanded to produce an expanded population of TILs, wherein 5 to 50% of the expanded population have a central memory T cell phenotype at day 14 of expansion. In some embodiments, the population of TILs is expanded to produce an expanded population of TILs, wherein 10 to 25% of the expanded population have a central memory T cell phenotype at day 14 of expansion. In some embodiments, the population of TILs is expanded to produce an expanded population of TILs, wherein 5 to 10% of the expanded population have a central memory T cell phenotype at day 14 of expansion. In some embodiments, the population of TILs is expanded to produce an expanded population of TILs, wherein 10 to 15% of the expanded population have a central memory T cell phenotype at day 14 of expansion. In some embodiments, the population of TILs is expanded to produce an expanded population of TILs, wherein 15 to 20% of the expanded population have a central memory T cell phenotype at day 14 of expansion. In some embodiments, the population of TILs is expanded to produce an expanded population of TILs, wherein 20 to 25% of the expanded population have a central memory T cell phenotype at day 14 of expansion. In some embodiments, the population of TILs is expanded to produce an expanded population of TILs, wherein 25 to 30% of the expanded population have a central memory T cell phenotype at day 14 of expansion. In some embodiments, the population of TILs is expanded to produce an expanded population of TILs, wherein 30 to 35% of the expanded population have a central memory T cell phenotype at day 14 of expansion. In some embodiments, the population of TILs is expanded to produce an expanded population of TILs, wherein 35 to 40% of the expanded population have a central memory T cell phenotype at day 14 of expansion. In some embodiments, the population of TILs is expanded to produce an expanded population of TILs, wherein 40 to 45% of the expanded population have a central memory T cell phenotype at day 14 of expansion. In some embodiments, the population of TILs is expanded to produce an expanded population of TILs, wherein 45 to 50% of the expanded population have a central memory T cell phenotype at day 14 of expansion.

In some embodiments, the population of TILs is expanded to produce an expanded population of TILs that have an increase in abundance of CD8+ cells. In some embodiments, the population of TILs is enriched 10% after expansion compared to the starting population of TILs. In some embodiments, the population of TILs is enriched 20% after expansion compared to the starting population of TILs. In some embodiments, the population of TILs is enriched 30% after expansion compared to the starting population of TILs. In some embodiments, the population of TILs is enriched 40% after expansion compared to the starting population of TILs. In some embodiments, the population of TILs is enriched 50% after expansion compared to the starting population of TILs. In some embodiments, the population of TILs is enriched 60% after expansion compared to the starting population of TILs. In some embodiments, the population of TILs is enriched 70% after expansion compared to the starting population of TILs. In some embodiments, the population of TILs is enriched 80% after expansion compared to the starting population of TILs. In some embodiments, the population of TILs is enriched 90% after expansion compared to the starting population of TILs. In some embodiments, the population of TILs is enriched 100% after expansion compared to the starting population of TILs.

In another aspect, the invention disclosed herein is directed to a composition comprising an expanded population of TILs produced by any of the methods disclosed herein.

### B. Phenotypic Characteristics of Expanded TILs

In some cases, the expanded TILs are analyzed for expression of numerous phenotype markers, including those described herein. In some cases, the marker is selected from: TCRα/β, CD57, CD28, CD4, CD27, CD56, CD8a, CD45RA, CD45RO, CD8a, CCR7, CD4, CD3, CD38, and HLA-DR. In some cases, expression of one or more regulatory markers is measured, namely from the group: CD137, CD8a, Lag3, CD4, CD3, PD-1, TIM-3, CD69, CD8a, TIGIT, CD4, CD3, KLRG1, and CD154.

In some cases, the memory marker is CCR7 or CD62L. In some cases, re-stimulated TILs can also be evaluated for cytokine release, using cytokine release assays. In some cases, TILs can be evaluated for interferon-gamma (IFN-gamma) secretion in response to stimulation either with OKT3 or co-culture with autologous tumor digest.

In some cases, TILs are evaluated for various regulatory markers, such as TCRα/β, CD56, CD27, CD28, CD57, CD45RA, CD45RO, CD25, CD127, CD95, IL-2R, CCR7, CD62L, KLRG1, and CD122.

### C. Genetic Modification of TILs

In some cases, the TILs are genetically engineered to include additional functionalities, including, but not limited to, a high-affinity T cell receptor (TCR), e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., EGFR, CD19 or HER2).

In some embodiments, the present disclosure provides modified TILs, encompassing TILs comprising one or more genomic modifications resulting in the reduced expression and/or function of one or more endogenous target genes as well as immune effector cells comprising a gene-regulating system capable of reducing the expression and/or function of one or more endogenous target genes. In some embodiments, these endogenous genes include ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.) In some embodiments, these genes include SOCS1, PTPN2, ZC3H12A, CBLB, RC3H1, NFKBIA. In some embodiments, these genes include SOCS1 and at least one, two or more genes selected from PTPN2, ZC3H12A, CBLB, RC3H, and NFKBIA.

Herein, the term “modified TIL” encompasses TILs comprising one or more genomic modifications, effected through non-natural means, resulting in the reduced expression and/or function of one or more endogenous target genes as well as TILs comprising a non-naturally occurring gene-regulating system capable of reducing the expression and/or function of one or more endogenous target genes. The term, “modified TIL” is used interchangeably with the terms “engineered TIL” or “eTIL™”

Herein, an “unmodified TIL” or “control TIL” refers to a cell or population of cells wherein the genomes have not been modified using exogenous means and that does not comprise an exogenous gene-regulating system or comprises a control gene-regulating system (e.g., an empty vector control, a non-targeting gRNA, a scrambled siRNA, etc.). Exemplary modifications that can be made to TILs are shown in International Publication Nos. WO 2019/178422, WO 2019/178420 and WO 2019/178421, incorporated by reference herein in their entireties. TILs that occur naturally that have reduced expression and/or function of one or more endogenous genes are included under the terms un-modified or control TILs.

Without wishing to be bound by theory, it is thought that TILs possess increased specificity to tumor antigens (Radvanyi et al., 2012 Clin Canc Res 18:6758-6770, incorporated herein by reference in its entirety) and can therefore mediate tumor antigen-specific immune response (e.g., activation, proliferation, and cytotoxic activity against the cancer cell) leading to cancer cell destruction (Brudno et al., 2018 Nat Rev Clin One 15:31-46, incorporated herein by reference in its entirety) without the introduction of an exogenous engineered receptor. Therefore, in some embodiments, TILs are isolated from a tumor in a subject, expanded ex vivo, and re-infused into a subject. In some embodiments, TILs are modified to express one or more exogenous receptors specific for an autologous tumor antigen, expanded ex vivo, and re-infused into the subject. Such embodiments can be modeled using in vivo mouse models wherein mice have been transplanted with a cancer cell line expressing a cancer antigen (e.g., CD19) and are treated with modified T cells that express an exogenous receptor that is specific for the cancer antigen.

In some embodiments, the modified TILs comprise one or more modifications (e.g., insertions, deletions, or mutations of one or more nucleic acids) in the genomic DNA sequence of an endogenous target gene resulting in the reduced expression and/or function the endogenous gene. Such modifications are referred to herein as “inactivating mutations” and endogenous genes comprising an inactivating mutation are referred to as “modified endogenous target genes.” In some embodiments, the inactivating mutations reduce or inhibit mRNA transcription, thereby reducing the expression level of the encoded mRNA transcript and protein. In some embodiments, the inactivating mutations reduce or inhibit mRNA translation, thereby reducing the expression level of the encoded protein. In some embodiments, the inactivating mutations encode a modified endogenous protein with reduced or altered function compared to the unmodified (i.e., wild-type) version of the endogenous protein (e.g., a dominant-negative mutant, described infra). Exemplary modifications that can be made to TILs are shown in International Publication Nos. WO 2019/178422, WO 2019/178420 and WO 2019/178421, incorporated by reference herein in their entireties. In some embodiments, the modified TILs comprise at least one, two or more modified endogenous target genes selected from SOCS1, PTPN2, ZC3H12A, CBLB, RC3H1 and NFKBIA In some embodiments, the modified TILs comprise the modified endogenous target gene SOCS1 and at least one, two or more modified endogenous target genes selected from PTPN2, ZC3H12A, CBLB, RC3H1, and NFKBIA.

In some embodiments, the modified TILs comprise one or more genomic modifications at a genomic location other than an endogenous target gene that result in the reduced expression and/or function of the endogenous target gene or that result in the expression of a modified version of an endogenous protein. For example, in some embodiments, a polynucleotide sequence encoding a gene regulating system is inserted into one or more locations in the genome, thereby reducing the expression and/or function of an endogenous target gene upon the expression of the gene-regulating system. In some embodiments, a polynucleotide sequence encoding a modified version of an endogenous protein is inserted at one or more locations in the genome, wherein the function of the modified version of the protein is reduced compared to the unmodified or wild-type version of the protein (e.g., a dominant-negative mutant, described infra).

In some embodiments, the modified TILs described herein comprise one or more modified endogenous target genes, wherein the one or more modifications result in reduced expression and/or function of a gene product (i.e., an mRNA transcript or a protein) encoded by the endogenous target gene compared to an unmodified TIL. For example, in some embodiments, a modified TIL demonstrates reduced expression of an mRNA transcript and/or reduced expression of a protein. In some embodiments, the expression of the gene product in a modified TIL is reduced by at least 5% compared to the expression of the gene product in an unmodified TIL. In some embodiments, the expression of the gene product in a modified TIL is reduced by at least 10%, 20%, 30%, 40%, 50%, 60%, 70%, 80%, 90%, or more compared to the expression of the gene product in an unmodified TIL. In some embodiments, the modified TILs described herein demonstrate reduced expression and/or function of gene products encoded by a plurality (e.g., two or more) of endogenous target genes compared to the expression of the gene products in an unmodified TIL. For example, in some embodiments, a modified TIL demonstrates reduced expression and/or function of gene products from 2, 3, 4, 5, 6, 7, 8, 9, 10, or more endogenous target genes compared to the expression of the gene products in an unmodified TIL.

In some embodiments, the present disclosure provides a modified TIL wherein one or more endogenous target genes, or a portion thereof, are deleted (i.e., “knocked-out”) such that the modified TIL does not express the mRNA transcript or protein. In some embodiments, a modified TIL comprises deletion of a plurality of endogenous target genes, or portions thereof. In some embodiments, a modified TIL comprises deletion of 2, 3, 4, 5, 6, 7, 8, 9, 10, or more endogenous target genes.

In some embodiments, the modified TILs described herein comprise one or more modified endogenous target genes, wherein the one or more modifications to the target DNA sequence result in expression of a protein with reduced or altered function (e.g., a “modified endogenous protein”) compared to the function of the corresponding protein expressed in an unmodified TIL (e.g., an “unmodified endogenous protein”). In some embodiments, the modified TILs described herein comprise 2, 3, 4, 5, 6, 7, 8, 9, 10, or more modified endogenous target genes encoding 2, 3, 4, 5, 6, 7, 8, 9, 10, or more modified endogenous proteins. In some embodiments, the modified endogenous protein demonstrates reduced or altered binding affinity for another protein expressed by the modified TIL or expressed by another cell; reduced or altered signaling capacity; reduced or altered enzymatic activity; reduced or altered DNA-binding activity; or reduced or altered ability to function as a scaffolding protein.

In some embodiments, the modified endogenous target gene comprises one or more dominant negative mutations. As used herein, a “dominant-negative mutation” refers to a substitution, deletion, or insertion of one or more nucleotides of a target gene such that the encoded protein acts antagonistically to the protein encoded by the unmodified target gene. The mutation is dominant-negative because the negative phenotype confers genic dominance over the positive phenotype of the corresponding unmodified gene. A gene comprising one or more dominant-negative mutations and the protein encoded thereby are referred to as a “dominant-negative mutants”, e.g., dominant-negative genes and dominant-negative proteins. In some embodiments, the dominant negative mutant protein is encoded by an exogenous transgene inserted at one or more locations in the genome of the TIL.

Various mechanisms for dominant negativity are known. Typically, the gene product of a dominant negative mutant retains some functions of the unmodified gene product but lacks one or more crucial other functions of the unmodified gene product. This causes the dominant-negative mutant to antagonize the unmodified gene product. For example, as an illustrative embodiment, a dominant-negative mutant of a transcription factor may lack a functional activation domain but retain a functional DNA binding domain. In this example, the dominant-negative transcription factor cannot activate transcription of the DNA as the unmodified transcription factor does, but the dominant-negative transcription factor can indirectly inhibit gene expression by preventing the unmodified transcription factor from binding to the transcription-factor binding site. As another illustrative embodiment, dominant-negative mutations of proteins that function as dimers are known. Dominant-negative mutants of such dimeric proteins may retain the ability to dimerize with unmodified protein but be unable to function otherwise. The dominant-negative monomers, by dimerizing with unmodified monomers to form heterodimers, prevent formation of functional homodimers of the unmodified monomers. Dominant negative mutations of the SOCS1 gene are known in the art and include the murine F59D mutant (See e.g., Hanada et al., J Biol Chem, 276:44:2 (2001), 40746-40754; and Suzuki et al., J Exp Med, 193:4 (2001), 471-482), and the human F58D mutant, identified by sequence alignments of the human and murine SOCS1 amino acid sequences.

In some embodiments, the modified TILs comprise a gene-regulating system capable of reducing the expression or function of one or more endogenous target genes. In some embodiments, the one or more target genes are selected from ANKRD11, BCL2L11, BCL3, BCOR, CALM2, CBLB, CHIC2, CTLA4, DHODH, E2F8, EGR2, FLI1, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.) In some embodiments, the modified TILs described herein comprise a gene-regulating system capable of reducing the expression and/or function of one or more endogenous target genes selected from SOCS1, PTPN2, ZC3H12A, CBLB, RC3H1 and NFKBIA. In some embodiments, the modified TILs described herein comprise a gene-regulating system capable of reducing the expression and/or function of one or more endogenous target genes selected from SOCS1 and at least one, two or more modified endogenous target genes selected from PTPN2, ZC3H12A, CBLB, RC3H1, and NFKBIA.

In some embodiments, the modified TILs described herein comprise a gene-regulating system capable of reducing the expression and/or function of two or more endogenous target genes. In some embodiments, the two or more target genes are selected from ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2DA, SMAD2, SOCS1, TANK, TGFBR1, TGFBR2, TIGIT, TNFAIP3, TNIP1, TRAF6, UMPS, WDR6 and ZC3H12A. In some embodiments, the modified TILs described herein comprise a gene-regulating system capable of reducing the expression and/or function of two or more endogenous target genes selected from SOCS1, PTPN2, ZC3H12A, CBLB, RC3H and NFKBIA. In some embodiments, the modified TILs described herein comprise a gene-regulating system capable of reducing the expression and/or function of SOCS1 and at least one, two or more modified endogenous target genes selected from PTPN2, ZC3H12A, CBLB, RC3H1, and NFKBIA. The gene-regulating system can reduce the expression and/or function of the endogenous target gene by a variety of mechanisms including by modifying the genomic DNA sequence of the endogenous target gene (e.g., by insertion, deletion, or mutation of one or more nucleic acids in the genomic DNA sequence); by regulating transcription of the endogenous target gene (e.g., inhibition or repression of mRNA transcription); and/or by regulating translation of the endogenous target gene (e.g., by mRNA degradation).

In some embodiments, the modified TILs described herein comprise a gene-regulating system (e.g., a nucleic acid-based gene-regulating system, a protein-based gene-regulating system, or a combination protein/nucleic acid-based gene-regulating system). In such embodiments, the gene-regulating system comprised in the modified TIL is capable of modifying one or more endogenous target genes. In some embodiments, the modified TILs described herein comprise a gene-regulating system comprising:
a. one or more nucleic acid molecules capable of reducing the expression or modifying the function of a gene product encoded by one or more endogenous target genes;
b. one or more polynucleotides encoding a nucleic acid molecule that is capable of reducing the expression or modifying the function of a gene product encoded by one or more endogenous target genes;
c. one or more proteins capable of reducing the expression or modifying the function of a gene product encoded by one or more endogenous target genes;
d. one or more polynucleotides encoding a protein that is capable of reducing the expression or modifying the function of a gene product encoded by one or more endogenous target genes;
e. one or more guide RNAs (gRNAs) capable of binding to a target DNA sequence in an endogenous gene;
f. one or more polynucleotides encoding one or more gRNAs capable of binding to a target DNA sequence in an endogenous gene;
g. one or more site-directed modifying polypeptides capable of interacting with a gRNA and modifying a target DNA sequence in an endogenous gene;
h. one or more polynucleotides encoding a site-directed modifying polypeptide capable of interacting with a gRNA and modifying a target DNA sequence in an endogenous gene;
i. one or more guide DNAs (gDNAs) capable of binding to a target DNA sequence in an endogenous gene;
j. one or more polynucleotides encoding one or more gDNAs capable of binding to a target DNA sequence in an endogenous gene;
k. one or more site-directed modifying polypeptides capable of interacting with a gDNA and modifying a target DNA sequence in an endogenous gene;
l. one or more polynucleotides encoding a site-directed modifying polypeptide capable of interacting with a gDNA and modifying a target DNA sequence in an endogenous gene;
m. one or more gRNAs capable of binding to a target mRNA sequence encoded by an endogenous gene;
n. one or more polynucleotides encoding one or more gRNAs capable of binding to a target mRNA sequence encoded by an endogenous gene;
o. one or more site-directed modifying polypeptides capable of interacting with a gRNA and modifying a target mRNA sequence encoded by an endogenous gene;
p. one or more polynucleotides encoding a site-directed modifying polypeptide capable of interacting with a gRNA and modifying a target mRNA sequence encoded by an endogenous gene; or
q. any combination of the above.

In some embodiments, the modified TILs described herein comprise a gene-regulating system comprising:
a. one or more nucleic acid molecules capable of reducing the expression and/or modifying the function of a gene product encoded by one or more endogenous target genes selected from SOCS1, PTPN2, ZC3H12A, CBLB, RC3H1 and NFKBIA;
b. one or more polynucleotides encoding one or more nucleic acid molecules that are capable of reducing the expression and/or modifying the function of the gene products encoded by one or more endogenous target genes selected from SOCS1, PTPN2, ZC3H12A, CBLB, RC3H1 and NFKBIA;
c. one or more proteins capable of reducing the expression and/or modifying the function of the gene products encoded by one or more endogenous target genes selected from SOCS1, PTPN2, ZC3H12A, CBLB, RC3H1 and NFKBIA;
d. one or more polynucleotides encoding one or more proteins that are capable of reducing the expression and/or modifying the function of a gene product encoded by one or more endogenous target genes selected from SOCS1, PTPN2, ZC3H12A, CBLB, RC3H and NFKBIA;
e. one or more guide RNAs (gRNAs) capable of binding to a target DNA sequence in one or more endogenous genes selected from SOCS1, PTPN2, ZC3H12A, CBLB, RC3H and NFKBIA;
f. one or more polynucleotides encoding one or more gRNAs capable of binding to a target DNA sequence in one or more endogenous genes selected from SOCS1, PTPN2, ZC3H12A, CBLB, RC3H and NFKBIA;
g. one or more site-directed modifying polypeptides capable of interacting with a gRNA and modifying a target DNA sequence in an endogenous gene selected from SOCS1, PTPN2, ZC3H12A, CBLB, RC3H and NFKBIA;
h. one or more polynucleotides encoding a site-directed modifying polypeptide capable of interacting with a gRNA and modifying a target DNA sequence in an endogenous gene selected from SOCS1, PTPN2, ZC3H12A, CBLB, RC3H and NFKBIA;
i. one or more guide DNAs (gDNAs) capable of binding to a target DNA sequence in two or more endogenous genes selected from SOCS1, PTPN2, ZC3H12A, CBLB, RC3H and NFKBIA;
j. one or more polynucleotides encoding one or more gDNAs capable of binding to a target DNA sequence in two or more endogenous genes selected from SOCS1, PTPN2, ZC3H12A, CBLB, RC3H and NFKBIA;
k. one or more site-directed modifying polypeptides capable of interacting with a gDNA and modifying a target DNA sequence in an endogenous gene selected from SOCS1, PTPN2, ZC3H12A, CBLB, RC3H and NFKBIA;
l. one or more polynucleotides encoding a site-directed modifying polypeptide capable of interacting with a gDNA and modifying a target DNA sequence in an endogenous gene selected from SOCS1, PTPN2, ZC3H12A, CBLB, RC3H and NFKBIA;
m. one or more gRNAs capable of binding to a target mRNA sequence encoded by one or more endogenous genes selected from SOCS1, PTPN2, ZC3H12A, CBLB, RC3H and NFKBIA;
n. one or more polynucleotides encoding one or more gRNAs capable of binding to a target mRNA sequence encoded by two or more endogenous genes selected from SOCS1, PTPN2, ZC3H12A, CBLB, RC3H1 and NFKBIA;
o. one or more site-directed modifying polypeptides capable of interacting with a gRNA and modifying a target mRNA sequence encoded by an endogenous gene selected from SOCS1, PTPN2, ZC3H12A, CBLB, RC3H1 and NFKBIA;
p. one or more polynucleotides encoding a site-directed modifying polypeptide capable of interacting with a gRNA and modifying a target mRNA sequence encoded by an endogenous gene selected from SOCS1, PTPN2, ZC3H12A, CBLB, RC3H1 and NFKBIA; or
q. any combination of the above.

In some embodiments, the modified TILs described herein comprise a gene-regulating system comprising:
a. two or more nucleic acid molecules capable of reducing the expression and/or modifying the function of a gene product encoded by two or more endogenous target genes selected from SOCS1, PTPN2, ZC3H12A, CBLB, RC3H1 and NFKBIA;
b. one or more polynucleotides encoding two or more nucleic acid molecules that are capable of reducing the expression and/or modifying the function of the gene products encoded by two or more endogenous target genes selected from SOCS1, PTPN2, ZC3H12A, CBLB, RC3H1 and NFKBIA;
c. two or more proteins capable of reducing the expression and/or modifying the function of the gene products encoded by two or more endogenous target genes selected from SOCS1, PTPN2, ZC3H12A, CBLB, RC3H1 and NFKBIA;
d. one or more polynucleotides encoding two or more proteins that are capable of reducing the expression and/or modifying the function of a gene product encoded by two or more endogenous target genes selected from SOCS1, PTPN2, ZC3H12A, CBLB, RC3H1 and NFKBIA;
e. two or more guide RNAs (gRNAs) capable of binding to a target DNA sequence in two or more endogenous genes selected from SOCS1, PTPN2, ZC3H12A, CBLB, RC3H and NFKBIA;
f. one or more polynucleotides encoding two or more gRNAs capable of binding to a target DNA sequence in two or more endogenous genes selected from SOCS1, PTPN2, ZC3H12A, CBLB, RC3H and NFKBIA;
g. two or more guide DNAs (gDNAs) capable of binding to a target DNA sequence in two or more endogenous genes selected from SOCS1, PTPN2, ZC3H12A, CBLB, RC3H and NFKBIA;
h. one or more polynucleotides encoding two or more gDNAs capable of binding to a target DNA sequence in two or more endogenous genes selected from SOCS1, PTPN2, ZC3H12A, CBLB, RC3H and NFKBIA;
i. two or more gRNAs capable of binding to a target mRNA sequence encoded by two or more endogenous genes selected from SOCS1, PTPN2, ZC3H12A, CBLB, RC3H and NFKBIA;
j. one or more polynucleotides encoding two or more gRNAs capable of binding to a target mRNA sequence encoded by two or more endogenous genes selected from SOCS1, PTPN2, ZC3H12A, CBLB, RC3H and NFKBIA;
k. any combination of the above.

In some embodiments, one, two or more polynucleotides encoding the gene-regulating system is inserted into the genome of the TIL. In some embodiments, one or more polynucleotides encoding the gene-regulating system is expressed episomally and is not inserted into the genome of the TIL.

In some embodiments, the modified TILs described herein comprise reduced expression and/or function of one or more endogenous target genes and further comprise one or more exogenous transgenes inserted at one or more genomic loci (e.g., a genetic “knock-in”). In some embodiments, the one or more exogenous transgenes encode detectable tags, safety-switch systems, chimeric switch receptors, and/or engineered antigen-specific receptors.

In some embodiments, the modified TILs described herein further comprise an exogenous transgene encoding a detectable tag. Examples of detectable tags include but are not limited to, FLAG tags, poly-histidine tags (e.g., 6×His), SNAP tags, Halo tags, cMyc tags, glutathione-S-transferase tags, avidin, enzymes, fluorescent proteins, luminescent proteins, chemiluminescent proteins, bioluminescent proteins, and phosphorescent proteins. In some embodiments the fluorescent protein is selected from the group consisting of blue/UV proteins (such as BFP, TagBFP, mTagBFP2, Azurite, EBFP2, mKalama1, Sirius, Sapphire, and T-Sapphire); cyan proteins (such as CFP, eCFP, Cerulean, SCFP3A, mTurquoise, mTurquoise2, monomeric Midoriishi-Cyan, TagCFP, and mTFP1); green proteins (such as: GFP, eGFP, meGFP (A208K mutation), Emerald, Superfolder GFP, Monomeric Azami Green, TagGFP2, mUKG, mWasabi, Clover, and mNeonGreen); yellow proteins (such as YFP, eYFP, Citrine, Venus, SYFP2, and TagYFP); orange proteins (such as Monomeric Kusabira-Orange, mKOκ, mKO2, mOrange, and mOrange2); red proteins (such as RFP, mRaspberry, mCherry, mStrawberry, mTangerine, tdTomato, TagRFP, TagRFP-T, mApple, mRuby, and mRuby2); far-red proteins (such as mPlum, HcRed-Tandem, mKate2, mNeptune, and NirFP); near-infrared proteins (such as TagRFP657, IFP1.4, and iRFP); long stokes shift proteins (such as mKeima Red, LSS-mKate1, LSS-mKate2, and mBeRFP); photoactivatible proteins (such as PA-GFP, PAmCherry1, and PATagRFP); photoconvertible proteins (such as Kaede (green), Kaede (red), KikGR1 (green), KikGR1 (red), PS-CFP2, PS-CFP2, mEos2 (green), mEos2 (red), mEos3.2 (green), mEos3.2 (red), PSmOrange, and PSmOrange); and photoswitchable proteins (such as Dronpa). In some embodiments, the detectable tag can be selected from AmCyan, AsRed, DsRed2, DsRed Express, E2-Crimson, HcRed, ZsGreen, ZsYellow, mCherry, mStrawberry, mOrange, mBanana, mPlum, mRasberry, tdTomato, DsRed Monomer, and/or AcGFP, all of which are available from Clontech.

In some embodiments, the modified TILs described herein further comprise an exogenous transgene encoding a safety-switch system. Safety-switch systems (also referred to in the art as suicide gene systems) comprise exogenous transgenes encoding for one or more proteins that enable the elimination of a modified TIL after the cell has been administered to a subject. Examples of safety-switch systems are known in the art. For example, safety-switch systems include genes encoding for proteins that convert non-toxic pro-drugs into toxic compounds such as the Herpes simplex thymidine kinase (Hsv-tk) and ganciclovir (GCV) system (Hsv-tk/GCV). Hsv-tk converts non-toxic GCV into a cytotoxic compound that leads to cellular apoptosis. As such, administration of GCV to a subject that has been treated with modified TILs comprising a transgene encoding the Hsv-tk protein can selectively eliminate the modified TILs while sparing endogenous TILs. See e.g., Bonini et al., Science, 1997, 276(5319):1719-1724; Ciceri et al., Blood, 2007, 109(11):1828-1836; Bondanza et al., Blood 2006, 107(5):1828-1836, incorporated herein by reference in their entireties.

Additional safety-switch systems include genes encoding for cell-surface markers, enabling elimination of modified TILs by administration of a monoclonal antibody specific for the cell-surface marker via ADCC. In some embodiments, the cell-surface marker is CD20 and the modified TILs can be eliminated by administration of an anti-CD20 monoclonal antibody such as Rituximab (see e.g., Introna et al., Hum Gene Ther, 2000, 11(4):611-620; Serafini et al., Hum Gene Ther, 2004, 14, 63-76; van Meerten et al., Gene Ther, 2006, 13, 789-797, incorporated herein by reference in their entireties). Similar systems using EGF-R and Cetuximab or Panitumumab are described in International PCT Publication No. WO 2018006880, incorporated herein by reference in its entirety. Additional safety-switch systems include transgenes encoding pro-apoptotic molecules comprising one or more binding sites for a chemical inducer of dimerization (CID), enabling elimination of modified TILs by administration of a CID which induces oligomerization of the pro-apoptotic molecules and activation of the apoptosis pathway. In some embodiments, the pro-apoptotic molecule is Fas (also known as CD95) (Thomis et al., Blood, 2001, 97(5), 1249-1257, incorporated herein by reference in its entirety). In some embodiments, the pro-apoptotic molecule is caspase-9 (Straathof et al., Blood, 2005, 105(11), 4247-4254, incorporated herein by reference in its entirety).

In some embodiments, the modified TILs described herein further comprise an exogenous transgene encoding a chimeric switch receptor. Chimeric switch receptors are engineered cell-surface receptors comprising an extracellular domain from an endogenous cell-surface receptor and a heterologous intracellular signaling domain, such that ligand recognition by the extracellular domain results in activation of a different signaling cascade than that activated by the wild type form of the cell-surface receptor. In some embodiments, the chimeric switch receptor comprises the extracellular domain of an inhibitory cell-surface receptor fused to an intracellular domain that leads to the transmission of an activating signal rather than the inhibitory signal normally transduced by the inhibitory cell-surface receptor. In particular embodiments, extracellular domains derived from cell-surface receptors known to inhibit TIL activation can be fused to activating intracellular domains. Engagement of the corresponding ligand will then activate signaling cascades that increase, rather than inhibit, the activation of the TIL. For example, in some embodiments, the modified TILs described herein comprise a transgene encoding a PD1-CD28 switch receptor, wherein the extracellular domain of PD1 is fused to the intracellular signaling domain of CD28 (see e.g., Liu et al., Cancer Res 76:6 (2016), 1578-1590 and Moon et al., Molecular Therapy 22 (2014), S201, incorporated herein by reference in its entirety). In some embodiments, the modified TILs described herein comprise a transgene encoding the extracellular domain of CD200R and the intracellular signaling domain of CD28 (see Oda et al., Blood 130:22 (2017), 2410-2419, incorporated herein by reference in its entirety). In some embodiments, the modified TILs described herein further comprise an engineered antigen-specific receptor recognizing a protein target expressed by a target cell, such as a tumor cell or an antigen presenting cell (APC), referred to herein as “modified receptor-engineered cells” or “modified RE-cells”. The term “engineered antigen receptor” refers to a non-naturally occurring antigen-specific receptor such as a chimeric antigen receptor (CAR) or a recombinant T cell receptor (TCR). In some embodiments, the engineered antigen receptor is a CAR comprising an extracellular antigen binding domain fused via hinge and transmembrane domains to a cytoplasmic domain comprising a signaling domain. In some embodiments, the CAR extracellular domain binds to an antigen expressed by a target cell in an MHC-independent manner leading to activation and proliferation of the RE cell. In some embodiments, the extracellular domain of a CAR recognizes a tag fused to an antibody or antigen-binding fragment thereof. In such embodiments, the antigen-specificity of the CAR is dependent on the antigen-specificity of the labeled antibody, such that a single CAR construct can be used to target multiple different antigens by substituting one antibody for another (See e.g., U.S. Pat. Nos. 9,233,125 and 9,624,279; US Patent Application Publication Nos. 20150238631 and 20180104354). In some embodiments, the extracellular domain of a CAR may comprise an antigen binding fragment derived from an antibody. Antigen binding domains that are useful in the present disclosure include, for example, scFvs; antibodies; antigen binding regions of antibodies; variable regions of the heavy/light chains; and single chain antibodies.

In some embodiments, the intracellular signaling domain of a CAR may be derived from the TCR complex zeta chain (such as CD3 signaling domains), FcγRIII, FcεRI, or the T-lymphocyte activation domain. In some embodiments, the intracellular signaling domain of a CAR further comprises a costimulatory domain, for example a 4-1BB, CD28, CD40, MyD88, or CD70 domain. In some embodiments, the intracellular signaling domain of a CAR comprises two costimulatory domains, for example any two of 4-1BB, CD28, CD40, MyD88, or CD70 domains. Exemplary CAR structures and intracellular signaling domains are known in the art (See e.g., WO 2009/091826; US 20130287748; WO 2015/142675; WO 2014/055657; and WO 2015/090229, incorporated herein by reference).

CARs specific for a variety of tumor antigens are known in the art, for example CD171-specific CARs (Park et al., Mol Ther (2007) 15(4):825-833), EGFRvIII-specific CARs (Morgan et al., Hum Gene Ther (2012) 23(10):1043-1053), EGF-R-specific CARs (Kobold et al., J Natl Cancer Inst (2014) 107(1):364), carbonic anhydrase K-specific CARs (Lamers et al., Biochem Soc Trans (2016) 44(3):951-959), FR-a-specific CARs (Kershaw et al., Clin Cancer Res (2006) 12(20):6106-6015), HER2-specific CARs (Ahmed et al., J Clin Oncol (2015) 33(15)1688-1696; Nakazawa et al., Mol Ther (2011) 19(12):2133-2143; Ahmed et al., Mol Ther (2009) 17(10):1779-1787; Luo et al., Cell Res (2016) 26(7):850-853; Morgan et al., Mol Ther (2010) 18(4):843-851; Grada et al., Mol Ther Nucleic Acids (2013) 9(2):32), CEA-specific CARs (Katz et al., Clin Cancer Res (2015) 21(14):3149-3159), IL13Ra2-specific CARs (Brown et al., Clin Cancer Res (2015) 21(18):4062-4072), GD2-specific CARs (Louis et al., Blood (2011) 118(23):6050-6056; Caruana et al., Nat Med (2015) 21(5):524-529), ErbB2-specific CARs (Wilkie et al., J Clin Immunol (2012) 32(5):1059-1070), VEGF-R-specific CARs (Chinnasamy et al., Cancer Res (2016) 22(2):436-447), FAP-specific CARs (Wang et al., Cancer Immunol Res (2014) 2(2):154-166), MSLN-specific CARs (Moon et al, Clin Cancer Res (2011) 17(14):4719-30), NKG2D-specific CARs (VanSeggelen et al., Mol Ther (2015) 23(10):1600-1610), CD19-specific CARs (Axicabtagene ciloleucel (Yescarta*) and Tisagenlecleucel (Kymriah®). See also, Li et al., J Hematol and Oncol (2018) 11(22), reviewing clinical trials of tumor-specific CARs. Exemplary CARs suitable for use according to the present disclosure are described below in Table 2.

TABLE 2
Exemplary CAR constructs

| | | Ag- | Intra- | Trans- | AA | NA |
|---|---|---|---|---|---|---|
| CAR | | binding | cellular | membrane | SEQ | SEQ |
| Ref ID | Target | domain | Domain | Domain | ID | ID |
| KSQCAR017 | human | Cetuximab | CD3 | CD8a | 903 | 904 |
| | EGFR | H225 scFv | zeta | hinge | | |
| KSQCAR1909 | human | FMC63 | CD3 | CD8a | 905 | 906 |
| | CD19 | scFv | zeta | hinge | | |
| KSQCAR010 | human | Herceptin | CD3 | CD8a | 907 | 908 |
| | HER2 | scFv | zeta | hinge | | |

In some embodiments, the engineered antigen receptor is a recombinant TCR. Recombinant TCRs comprise TCRα and/or TCRβ chains that have been isolated and cloned from T cell populations recognizing a particular target antigen. For example, TCRα and/or TCRβ genes (i.e., TRAC and TRBC) can be cloned from T cell populations isolated from individuals with particular malignancies or T cell populations that have been isolated from humanized mice immunized with specific tumor antigens or tumor cells. Recombinant TCRs recognize antigen through the same mechanisms as their endogenous counterparts (e.g., by recognition of their cognate antigen presented in the context of major histocompatibility complex (MHC) proteins expressed on the surface of a target cell). This antigen engagement stimulates endogenous signal transduction pathways leading to activation and proliferation of the TCR-engineered cells.

Recombinant TCRs specific for tumor antigens are known in the art, for example WT1-specific TCRs (JTCR016, Juno Therapeutics; WT1-TCRc4, described in US Patent Application Publication No. 20160083449), MART-1 specific TCRs (including the DMF4T clone, described in Morgan et al., Science 314 (2006) 126-129); the DMF5T clone, described in Johnson et al., Blood 114 (2009) 535-546); and the ID3T clone, described in van den Berg et al., Mol. Ther. 23 (2015) 1541-1550), gp100-specific TCRs (Johnson et al., Blood 114 (2009) 535-546), CEA-specific TCRs (Parkhurst et al., Mol Ther. 19 (2011) 620-626), NY-ESO and LAGE-1 specific TCRs (1G4T clone, described in Robbins et al., J Clin Oncol 26 (2011) 917-924; Robbins et al., Clin Cancer Res 21 (2015) 1019-1027; and Rapoport et al., Nature Medicine 21 (2015) 914-921), and MAGE-A3-specific TCRs (Morgan et al., J Immunother 36 (2013) 133-151) and Linette et al., Blood 122 (2013) 227-242). (See also, Debets et al., Seminars in Immunology 23 (2016) 10⁻²¹).

To generate the recombinant TCRs, the native TRAC (SEQ ID NO: 882) and TRBC (SEQ ID NOs: 883) protein sequences are fused to the C-terminal ends of TCR-α and TCR-β chain variable regions specific for a protein or peptide of interest. For example, the engineered TCR can recognize the NY-ESO peptide (SLLMWITQC, SEQ ID NO: 884), such as the 1G4 TCR or the 95:LY TCR (Robbins et al, Journal of Immunology 2008 180:6116-6131). In such illustrative embodiments, the paired 1G4-TCR α/βchains comprise SEQ ID NOs: 885 and 886, respectively and the paired 95:LY-TCR α/βchains comprise SEQ ID NOs: 887 and 888, respectively. The recombinant TCR can recognize the MART-1 peptide (AAGIGILTV, SEQ ID NO: 889), such as the DMF4 and DMF5 TCRs (Robbins et al, Journal of Immunology 2008 180:6116-6131). In such illustrative embodiments, the paired DMF4-TCR α/βchains comprise SEQ ID NOs: 890 and 891, respectively and the paired DMF5-TCR α/βchains comprise SEQ ID NOs: 892 and 893, respectively. The recombinant TCR can recognize the WT-1 peptide (RMFPNAPYL, SEQ ID NO: 894), such as the DLT TCR (Robbins et al, Journal of Immunology 2008 180:6116-6131). In such illustrative embodiments, the paired high-affinity DLT-TCR w/chains comprise SEQ ID NOs: 895 and 896, respectively.

Codon-optimized DNA sequences encoding the recombinant TCRα and TCRβ chain proteins can be generated such that expression of both TCR chains is driven off of a single promoter in a stoichiometric fashion. In such embodiment, the P2A sequence (SEQ ID NO: 897) can be inserted between the DNA sequences encoding the TCRβ and the TCRα chain, such that the expression cassettes encoding the recombinant TCR chains comprise the following format: TCRβ-P2A-TCRα. As an illustrative embodiment, the protein sequence of the 1G4 NY-ESO-specific TCR expressed from such a cassette would comprise SEQ ID NO: 898, the protein sequence of the 95:LY NY-ESO-specific TCR expressed from such a cassette would comprise SEQ ID NO: 899, the protein sequence of the DMF4 MART1-specific TCR expressed from such a cassette would comprise SEQ ID NO: 900, the protein sequence of the DMF5 MART1-specific TCR expressed from such a cassette would comprise SEQ ID NO: 901, and the protein sequence of the DLT WT1-specific TCR expressed from such a cassette would comprise SEQ ID NO: 902.

In some embodiments, the engineered antigen receptor is directed against a target antigen selected from a cluster of differentiation molecule, such as CD3, CD4, CD8, CD16, CD24, CD25, CD33, CD34, CD45, CD64, CD71, CD78, CD80 (also known as B7-1), CD86 (also known as B7-2), CD96, , CD116, CD117, CD123, CD133, and CD138, CD371 (also known as CLL1); a tumor-associated surface antigen, such as 5T4, BCMA (also known as CD269 and TNFRSF17, UniProt #Q02223), carcinoembryonic antigen (CEA), carbonic anhydrase 9 (CAIX or MN/CAIX), CD19, CD20, CD22, CD30, CD40, disialogangliosides such as GD2, ELF2M, ductal-epithelial mucin, ephrin B2, epithelial cell adhesion molecule (EpCAM), ErbB2 (HER2/neu), FCRL5 (UniProt #Q68SN8), FKBP11 (UniProt #Q9NYL4), glioma-associated antigen, glycosphingolipids, gp36, GPRC5D (UniProt #Q9NZD1), mut hsp70-2, intestinal carboxyl esterase, IGF-I receptor, ITGA8 (UniProt #P53708), KAMP3, LAGE-1a, MAGE, mesothelin, neutrophil elastase, NKG2D, Nkp30, NY-ESO-1, PAP, prostase, prostate-carcinoma tumor antigen-1 (PCTA-1), prostate specific antigen (PSA), PSMA, prostein, RAGE-1, ROR1, RU1 (SFMBT1), RU2 (DCDC2), SLAMF7 (UniProt #Q9NQ25), survivin, TAG-72, and telomerase; a major histocompatibility complex (MHC) molecule presenting a tumor-specific peptide epitope; tumor stromal antigens, such as the extra domain A (EDA) and extra domain B (EDB) of fibronectin; the A1 domain of tenascin-C(TnC A1) and fibroblast associated protein (FAP); cytokine receptors, such as epidermal growth factor receptor (EGFR), EGFR variant III (EGFRvIII), TFGβ-R or components thereof such as endoglin; a major histocompatibility complex (MHC) molecule; a virus-specific surface antigen such as an HIV-specific antigen (such as HIV gp120); an EBV-specific antigen, a CMV-specific antigen, a HPV-specific antigen, a Lassa virus-specific antigen, an Influenza virus-specific antigen as well as any derivate or variant of these surface antigens.

In some embodiments, the present disclosure provides modified TILs comprising reduced expression and/or function of one, two or more endogenous target genes. In some embodiments, these endogenous genes include ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.)

In some embodiments, the present disclosure provides modified TILs comprising reduced expression and/or function of SOCS1 and PTPN2 or a gene-regulating system capable of reducing the expression and/or function of SOCS1 and PTPN2 and further comprising a CAR or recombinant TCR expressed on the cell surface. In some embodiments, the modified TILs comprise reduced expression and/or function of SOCS1 and PTPN2 or a gene-regulating system capable of reducing the expression and/or function of SOCS1 and PTPN2 and further comprising a recombinant expression vector encoding a CAR or a recombinant TCR.

In some embodiments, the present disclosure provides modified TILs comprising reduced expression and/or function of SOCS1 and ZC3H12A or a gene-regulating system capable of reducing the expression and/or function of SOCS1 and ZC3H12A and further comprising a CAR or recombinant TCR expressed on the cell surface. In some embodiments, the modified TILs comprise reduced expression and/or function of SOCS1 and ZC3H12A or a gene-regulating system capable of reducing the expression and/or function of SOCS1 and ZC3H12A and further comprising a recombinant expression vector encoding a CAR or a recombinant TCR.

In some embodiments, the present disclosure provides modified TILs comprising reduced expression and/or function of PTPN2 and ZC3H12A or a gene-regulating system capable of reducing the expression and/or function of PTPN2 and ZC3H12A and further comprising a CAR or recombinant TCR expressed on the cell surface. In some embodiments, the modified TILs comprise reduced expression and/or function of PTPN2 and ZC3H12A or a gene-regulating system capable of reducing the expression and/or function of PTPN2 and ZC3H12A and further comprising a recombinant expression vector encoding a CAR or a recombinant TCR.

In some embodiments, the present disclosure provides modified TILs comprising reduced expression and/or function of PTPN2 and CBLB or a gene-regulating system capable of reducing the expression and/or function of PTPN2 and CBLB and further comprising a CAR or recombinant TCR expressed on the cell surface. In some embodiments, the modified TILs comprise reduced expression and/or function of PTPN2 and CBLB or a gene-regulating system capable of reducing the expression and/or function of PTPN2 and CBLB and further comprising a recombinant expression vector encoding a CAR or a recombinant TCR.

In some embodiments, the present disclosure provides modified TILs comprising reduced expression and/or function of ZC3H12A and CBLB or a gene-regulating system capable of reducing the expression and/or function of ZC3H12A and CBLB and further comprising a CAR or recombinant TCR expressed on the cell surface. In some embodiments, the modified TILs comprise reduced expression and/or function of ZC3H12A and CBLB or a gene-regulating system capable of reducing the expression and/or function of ZC3H12A and CBLB and further comprising a recombinant expression vector encoding a CAR or a recombinant TCR.

In some embodiments, the present disclosure provides modified TILs comprising reduced expression and/or function of SOCS1 and CBLB or a gene-regulating system capable of reducing the expression and/or function of SOCS1 and CBLB and further comprising a CAR or recombinant TCR expressed on the cell surface. In some embodiments, the modified TILs comprise reduced expression and/or function of SOCS1 and CBLB or a gene-regulating system capable of reducing the expression and/or function of SOCS1 and CBLB and further comprising a recombinant expression vector encoding a CAR or a recombinant TCR.

In some embodiments, the present disclosure provides modified TILs comprising reduced expression and/or function of PTPN2 and RC3H1 or a gene-regulating system capable of reducing the expression and/or function of PTPN2 and RC3H1 and further comprising a CAR or recombinant TCR expressed on the cell surface. In some embodiments, the modified TILs comprise reduced expression and/or function of PTPN2 and RC3H1 or a gene-regulating system capable of reducing the expression and/or function of PTPN2 and RC3H1 and further comprising a recombinant expression vector encoding a CAR or a recombinant TCR.

In some embodiments, the present disclosure provides modified TILs comprising reduced expression and/or function of ZC3H12A and RC3H1 or a gene-regulating system capable of reducing the expression and/or function of ZC3H12A and RC3H1 and further comprising a CAR or recombinant TCR expressed on the cell surface. In some embodiments, the modified TILs comprise reduced expression and/or function of ZC3H12A and RC3H1 or a gene-regulating system capable of reducing the expression and/or function of ZC3H12A and RC3H1 and further comprising a recombinant expression vector encoding a CAR or a recombinant TCR.

In some embodiments, the present disclosure provides modified TILs comprising reduced expression and/or function of SOCS1 and RC3H1 or a gene-regulating system capable of reducing the expression and/or function of SOCS1 and RC3H1 and further comprising a CAR or recombinant TCR expressed on the cell surface. In some embodiments, the modified TILs comprise reduced expression and/or function of SOCS1 and RC3H1 or a gene-regulating system capable of reducing the expression and/or function of SOCS1 and RC3H1 and further comprising a recombinant expression vector encoding a CAR or a recombinant TCR.

In some embodiments, the present disclosure provides modified TILs comprising reduced expression and/or function of CBLB and RC3H1 or a gene-regulating system capable of reducing the expression and/or function of CBLB and RC3H1 and further comprising a CAR or recombinant TCR expressed on the cell surface. In some embodiments, the modified TILs comprise reduced expression and/or function of CBLB and RC3H1 or a gene-regulating system capable of reducing the expression and/or function of CBLB and RC3H1 and further comprising a recombinant expression vector encoding a CAR or a recombinant TCR.

In some embodiments, the present disclosure provides modified TILs comprising reduced expression and/or function of PTPN2 and NFKBIA or a gene-regulating system capable of reducing the expression and/or function of PTPN2 and NFKBIA and further comprising a CAR or recombinant TCR expressed on the cell surface. In some embodiments, the modified TILs comprise reduced expression and/or function of PTPN2 and NFKBIA or a gene-regulating system capable of reducing the expression and/or function of PTPN2 and NFKBIA and further comprising a recombinant expression vector encoding a CAR or a recombinant TCR.

In some embodiments, the present disclosure provides modified TILs comprising reduced expression and/or function of ZC3H12A and NFKBIA or a gene-regulating system capable of reducing the expression and/or function of ZC3H12A and NFKBIA and further comprising a CAR or recombinant TCR expressed on the cell surface. In some embodiments, the modified TILs comprise reduced expression and/or function of ZC3H12A and NFKBIA or a gene-regulating system capable of reducing the expression and/or function of ZC3H12A and NFKBIA and further comprising a recombinant expression vector encoding a CAR or a recombinant TCR.

In some embodiments, the present disclosure provides modified TILs comprising reduced expression and/or function of SOCS1 and NFKBIA or a gene-regulating system capable of reducing the expression and/or function of SOCS1 and NFKBIA and further comprising a CAR or recombinant TCR expressed on the cell surface. In some embodiments, the modified TILs comprise reduced expression and/or function of SOCS1 and NFKBIA or a gene-regulating system capable of reducing the expression and/or function of SOCS1 and NFKBIA and further comprising a recombinant expression vector encoding a CAR or a recombinant TCR.

In some embodiments, the present disclosure provides modified TILs comprising reduced expression and/or function of CBLB and NFKBIA or a gene-regulating system capable of reducing the expression and/or function of CBLB and NFKBIA and further comprising a CAR or recombinant TCR expressed on the cell surface. In some embodiments, the modified TILs comprise reduced expression and/or function of CBLB and NFKBIA or a gene-regulating system capable of reducing the expression and/or function of CBLB and NFKBIA and further comprising a recombinant expression vector encoding a CAR or a recombinant TCR.

In some embodiments, the present disclosure provides modified TILs comprising reduced expression and/or function of RC3H1 and NFKBIA or a gene-regulating system capable of reducing the expression and/or function of RC3H1 and NFKBIA and further comprising a CAR or recombinant TCR expressed on the cell surface. In some embodiments, the modified TILs comprise reduced expression and/or function of RC3H1 and NFKBIA or a gene-regulating system capable of reducing the expression and/or function of RC3H1 and NFKBIA and further comprising a recombinant expression vector encoding a CAR or a recombinant TCR.

In some embodiments, the present disclosure provides modified TILs comprising a gene-regulating system capable of reducing the expression and/or function of one or more endogenous target genes. In some embodiments, these endogenous genes include ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2DA, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.)

In some embodiments, the present disclosure provides modified TILs comprising reduced expression and/or function of SOCS1 and PTPN2 or a gene-regulating system capable of reducing the expression and/or function of SOCS1 and PTPN2, wherein the immune effector cell is a TIL.

In some embodiments, the present disclosure provides modified TILs comprising reduced expression and/or function of SOCS1 and ZC3H12A or a gene-regulating system capable of reducing the expression and/or function of SOCS1 and ZC3H12A, wherein the immune effector cell is a TIL.

In some embodiments, the present disclosure provides modified TILs comprising reduced expression and/or function of PTPN2 and ZC3H12A or a gene-regulating system capable of reducing the expression and/or function of PTPN2 and ZC3H12A, wherein the immune effector cell is a TIL.

In some embodiments, the present disclosure provides modified TILs comprising reduced expression and/or function of PTPN2 and CBLB or a gene-regulating system capable of reducing the expression and/or function of PTPN2 and CBLB, wherein the immune effector cell is a TIL.

In some embodiments, the present disclosure provides modified TILs comprising reduced expression and/or function of ZC3H12A and CBLB or a gene-regulating system capable of reducing the expression and/or function of ZC3H12A and CBLB, wherein the immune effector cell is a TIL.

In some embodiments, the present disclosure provides modified TILs comprising reduced expression and/or function of SOCS1 and CBLB or a gene-regulating system capable of reducing the expression and/or function of SOCS1 and CBLB, wherein the immune effector cell is a TIL.

In some embodiments, the present disclosure provides modified TILs comprising reduced expression and/or function of PTPN2 and RC3H1 or a gene-regulating system capable of reducing the expression and/or function of PTPN2 and RC3H, wherein the immune effector cell is a TIL.

In some embodiments, the present disclosure provides modified TILs comprising reduced expression and/or function of ZC3H12A and RC3H1 or a gene-regulating system capable of reducing the expression and/or function of ZC3H12A and RC3H, wherein the immune effector cell is a TIL.

In some embodiments, the present disclosure provides modified TILs comprising reduced expression and/or function of SOCS1 and RC3H1 or a gene-regulating system capable of reducing the expression and/or function of SOCS1 and RC3H1, wherein the immune effector cell is a TIL.

In some embodiments, the present disclosure provides modified TILs comprising reduced expression and/or function of CBLB and RC3H1 or a gene-regulating system capable of reducing the expression and/or function of CBLB and RC3H1, wherein the immune effector cell is a TIL.

In some embodiments, the present disclosure provides modified TILs comprising reduced expression and/or function of PTPN2 and NFKBIA or a gene-regulating system capable of reducing the expression and/or function of PTPN2 and NFKBIA, wherein the immune effector cell is a TIL.

In some embodiments, the present disclosure provides modified TILs comprising reduced expression and/or function of ZC3H12A and NFKBIA or a gene-regulating system capable of reducing the expression and/or function of ZC3H12A and NFKBIA, wherein the immune effector cell is a TIL.

In some embodiments, the present disclosure provides modified TILs comprising reduced expression and/or function of SOCS1 and NFKBIA or a gene-regulating system capable of reducing the expression and/or function of SOCS1 and NFKBIA, wherein the immune effector cell is a TIL.

In some embodiments, the present disclosure provides modified TILs comprising reduced expression and/or function of CBLB and NFKBIA or a gene-regulating system capable of reducing the expression and/or function of CBLB and NFKBIA, wherein the immune effector cell is a TIL.

In some embodiments, the present disclosure provides modified TILs comprising reduced expression and/or function of RC3H1 and NFKBIA or a gene-regulating system capable of reducing the expression and/or function of RC3H1 and NFKBIA, wherein the immune effector cell is a TIL.

### D. Effector Functions

In some embodiments, the modified TILs described herein comprise reduced expression and/or function (or a gene-regulating system capable of reducing the expression and/or function) of one or more endogenous target genes selected from ANKRD11, BCL2L11, BCL3, BCOR, CALM2, CBLB, CHIC2, CTLA4, DHODH, E2F8, EGR2, FLI1, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A (See International Publication Nos. WO 2019/178422, WO 2019/178420 and WO 2019/178421, incorporated by reference herein in their entireties) and demonstrate an increase in one or more immune cell effector functions.

In some embodiments, the modified TILs described herein comprise reduced expression and/or function (or a gene-regulating system capable of reducing the expression and/or function) of one or more endogenous target genes selected from SOCS1, PTPN2, ZC3H12A, CBLB, RC3H1 and NFKBIA and demonstrate an increase in one or more immune cell effector functions. Herein, the term “effector function” refers to functions of an immune cell related to the generation, maintenance, and/or enhancement of an immune response against a target cell or target antigen. In some embodiments, the modified TILs described herein demonstrate one or more of the following characteristics compared to an unmodified TIL: increased infiltration or migration in to a tumor, increased proliferation, increased or prolonged cell viability, increased resistance to inhibitory factors in the surrounding microenvironment such that the activation state of the cell is prolonged or increased, increased production of pro-inflammatory immune factors (e.g., pro-inflammatory cytokines, chemokines, and/or enzymes), increased cytotoxicity, increased resistance to exhaustion and/or increased percentage of T_{cm}.

In some embodiments, the modified TILs described herein demonstrate increased infiltration into a tumor compared to an unmodified TIL. In some embodiments, increased tumor infiltration by modified TILs refers to an increase the number of modified TILs infiltrating into a tumor during a given period of time compared to the number of unmodified TILs that infiltrate into a tumor during the same period of time. In some embodiments, the modified TILs demonstrate a 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20,25, 30, 35, 40, 45, 50, 60, 70, 80, 90, 100, or more fold increase in tumor filtration compared to an unmodified immune cell. Tumor infiltration can be measured by isolating one or more tumors from a subject and assessing the number of modified immune cells in the sample by flow cytometry, immunohistochemistry, and/or immunofluorescence.

In some embodiments, the modified TILs described herein demonstrate an increase in cell proliferation compared to an unmodified TIL. In these embodiments, the result is an increase in the number of modified TILs present compared to unmodified TILs after a given period of time. For example, in some embodiments, modified TILs demonstrate increased rates of proliferation compared to unmodified TILs, wherein the modified TILs divide at a more rapid rate than unmodified TILs. In some embodiments, the modified TILs demonstrate a 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20,25, 30, 35, 40, 45, 50, 60, 70, 80, 90, 100, or more fold increase in the rate of proliferation compared to an unmodified immune cell. In some embodiments, modified TILs demonstrate prolonged periods of proliferation compared to unmodified TILs, wherein the modified TILs and unmodified TILs divide at similar rates, but wherein the modified TILs maintain the proliferative state for a longer period of time. In some embodiments, the modified TILs maintain a proliferative state for 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20,25, 30, 35, 40, 45, 50, 60, 70, 80, 90, 100, or more times longer than an unmodified immune cell.

In some embodiments, the modified TILs described herein demonstrate increased or prolonged cell viability compared to an unmodified TIL. In such embodiments, the result is an increase in the number of modified TILs or present compared to unmodified TILs after a given period of time. For example, in some embodiments, modified TILs described herein remain viable and persist for 1.1, 1.2, 1.3, 1.4, 1.5, 1.6, 1.7, 1.8, 1.9, 2, 2.5, 3, 3.5, 4, 4.5, 5, 6, 7, 8, 9, 10, 15, 20,25, 30, 35, 40, 45, 50, 60, 70, 80, 90, 100, or more times longer than an unmodified immune cell.

In some embodiments, the modified TILs described herein demonstrate increased resistance to inhibitory factors compared to an unmodified TIL. Exemplary inhibitory factors include signaling by immune checkpoint molecules (e.g., PD1, PDL1, CTLA4, LAG3, IDO) and/or inhibitory cytokines (e.g., IL-10, TGFβ).

In some embodiments, the modified T cells described herein demonstrate increased resistance to T cell exhaustion compared to an unmodified T cell. T cell exhaustion is a state of antigen-specific T cell dysfunction characterized by decreased effector function and leading to subsequent deletion of the antigen-specific T cells. In some embodiments, exhausted T cells lack the ability to proliferate in response to antigen, demonstrate decreased cytokine production, and/or demonstrate decreased cytotoxicity against target cells such as tumor cells. In some embodiments, exhausted T cells are identified by altered expression of cell surface markers and transcription factors, such as decreased cell surface expression of CD122 and CD127; increased expression of inhibitory cell surface markers such as PD1, LAG3, CD244, CD160, TIM3, and/or CTLA4; and/or increased expression of transcription factors such as Blimp1, NFAT, and/or BATF. In some embodiments, exhausted T cells demonstrate altered sensitivity cytokine signaling, such as increased sensitivity to TGFβ signaling and/or decreased sensitivity to IL-7 signaling. T cell exhaustion can be determined, for example, by co-culturing the T cells with a population of target cells and measuring T cell proliferation, cytokine production, and/or lysis of the target cells. In some embodiments, the modified TILs described herein are co-cultured with a population of target cells (e.g., autologous tumor cells or cell lines that have been engineered to express a target tumor antigen) and effector cell proliferation, cytokine production, and/or target cell lysis is measured. These results are then compared to the results obtained from co-culture of target cells with a control population of immune cells (such as unmodified TILs or immune effector cells that have a control modification).

In some embodiments, resistance to T cell exhaustion is demonstrated by increased production of one or more cytokines (e.g., IFNγ, TNFα, or IL-2) from the modified TILs compared to the cytokine production observed from the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in cytokine production from the modified TILs compared to the cytokine production from the control population of immune cells is indicative of an increased resistance to T cell exhaustion. In some embodiments, resistance to T cell exhaustion is demonstrated by increased proliferation of the modified TILs compared to the proliferation observed from the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in proliferation of the modified TILs compared to the proliferation of the control population of immune cells is indicative of an increased resistance to T cell exhaustion. In some embodiments, resistance to T cell exhaustion is demonstrated by increased target cell lysis by the modified TILs compared to the target cell lysis observed by the control population of immune cells. In some embodiments, a 1.1, 1.2, 1.3, 1.4, 1.5, 1.6, 1.7, 1.8, 1.9, 2.0, 2.5, 3.0, 3.5, 4.0, 4.5, 5, 6, 7, 8, 9, 10, 15, 20, 30, 35, 40, 45, 50, 60, 70, 80, 90, 100 or more fold increase in target cell lysis by the modified TILs compared to the target cell lysis by the control population of immune cells is indicative of an increased resistance to T cell exhaustion.

In some embodiments, exhaustion of the modified TILs compared to control populations of immune cells is measured during the in vitro or ex vivo manufacturing process. For example, in some embodiments, TILs isolated from tumor fragments are modified according to the methods described herein and then expanded in one or more rounds of expansion to produce a population of modified TILs. In such embodiments, the exhaustion of the modified TILs can be determined immediately after harvest and prior to a first round of expansion, after the first round of expansion but prior to a second round of expansion, and/or after the first and the second round of expansion. In some embodiments, exhaustion of the modified TILs compared to control populations of immune cells is measured at one or more time points after transfer of the modified TILs into a subject. For example, in some embodiments, the modified cells are produced according to the methods described herein and administered to a subject. Samples can then be taken from the subject at various time points after the transfer to determine exhaustion of the modified TILs in vivo over time.

In some embodiments, the modified TILs described herein demonstrate increased expression or production of pro-inflammatory immune factors compared to an unmodified TIL. Examples of pro-inflammatory immune factors include cytolytic factors, such as granzyme B, perforin, and granulysin; and pro-inflammatory cytokines such as interferons (IFNα, IFNβ, IFNγ), TNFα, IL-1β, IL-12, IL-2, IL-17, CXCL8, and/or IL-6.

In some embodiments, the modified TILs described herein demonstrate increased cytotoxicity against a target cell compared to an unmodified TIL. In some embodiments, the modified TILs demonstrate a 1, 2, 3, 4, 5, 6, 7, 8, 9, 10, 20, 30, 40, 50, 60, 70, 80, 90, 100, or more fold increase in cytotoxicity against a target cell compared to an unmodified immune cell.

Assays for measuring immune effector function are known in the art. For example, tumor infiltration can be measured by isolating tumors from a subject and determining the total number and/or phenotype of the lymphocytes present in the tumor by flow cytometry, immunohistochemistry, and/or immunofluorescence. Cell-surface receptor expression can be determined by flow cytometry, immunohistochemistry, immunofluorescence, Western blot, and/or qPCR. Cytokine and chemokine expression and production can be measured by flow cytometry, immunohistochemistry, immunofluorescence, Western blot, ELISA, and/or qPCR. Responsiveness or sensitivity to extracellular stimuli (e.g., cytokines, inhibitory ligands, or antigen) can be measured by assaying cellular proliferation and/or activation of downstream signaling pathways (e.g., phosphorylation of downstream signaling intermediates) in response to the stimuli. Cytotoxicity can be measured by target-cell lysis assays known in the art, including in vitro or ex vivo co-culture of the modified TILs with target cells and in vivo murine tumor models, such as those described throughout the Examples.

### E. Regulation of Endogenous Pathways and Genes

In some embodiments, the modified TILs described herein demonstrate a reduced expression and/or function of one, two or more endogenous target genes selected from SOCS1, PTPN2, ZC3H12A, CBLB, RC3H1 and NFKBIA. Further details on the endogenous target genes are provided below in Table 3. In such embodiments, the reduced expression or function of the one, two or more endogenous target genes enhances one or more effector functions of the immune cell.

In some embodiments, the modified TILs described herein comprise reduced expression and/or function of the Suppressors of cytokine signaling SOCS 1 (SOCS1) gene. The SOCS1 protein comprises C-terminal SOCS box motifs, an SH2-domain, an ESS domain, and an N-terminal KIR domain. The 12 amino-acid residues called the kinase inhibitory region (KIR) has been found to be critical in the ability of SOCS1 to negatively regulate JAK1, TYK2 and JAK2 tyrosine kinase function.

In some embodiments, the modified TILs described herein comprise reduced expression and/or function of the PTPN2 gene. The protein tyrosine phosphatase family (PTP) dephosphorylate phospho-tyrosine residues by their phosphatase catalytic domain. PTPN2 functions as a brake on both TCRs and cytokines, which signal through JAK/STAT signaling complexes, and thus serves as a checkpoint on both Signals 1 and 3. Following T Cell engagement with antigen and activation of the TCR, positive signals are amplified downstream by the kinases Lck and Fyn by phosphorylation of tyrosine residues. PTPN2 serves to dephosphorylate both Lck and Fyn and thus attenuate TCR signaling. In addition, following T cell encounter with cytokines and signaling through common γ chain receptor complex, which transmit positive signals though JAK/STAT signaling, PTPN2 also attenuates by dephosphorylation of STAT1 and STAT3. The sum functional impact of PTPN2 loss on T cell function is a lowering of the activation threshold needed for fulminant T cell activation through the TCR, and a hypersensitivity to growth and differentiation-enhancing cytokines.

In addition, deletion of PTPN2 in the whole mouse increases cytokine levels, lymphocytic infiltration in nonlymphoid tissues and early signs of rheumatoid arthritis-like symptoms; these mice do not survive past 5 weeks of age. Thus, PTPN2 has been identified as critical for postnatal development in mice. Consistent with this autoimmune phenotype, deletion of Ptpn2 in the T cell lineage from birth also results in an increase in lymphocytic infiltration in non-lymphoid tissues. Importantly, an inducible knockout of Ptpn2 in adult mouse T cells did not result in any autoimmune manifestations. Outside of its role in autoimmunity, Ptpn2 deletion was identified to associate with a small percentage of T cell acute lymphoblastic leukemia in humans (ALL), and to enhance skin tumor development in a two-stage chemically-induced carcinogenicity.

In some embodiments, the modified TILs described herein comprise reduced expression and/or function of the ZC3H12A gene. Zc3h12, also referred to as MCPIP1 and Regnase-1, is an RNase that possesses a RNase domain just upstream of a CCCH-type zinc-finger motif. Through its nuclease activity, Zc3h12a targets and destabilizes the mRNAs of transcripts, such as IL-6, by binding a conserved stem loop structure within the 3′ UTR of these genes. In T cells, Zc3h12a controls the transcript levels of a number of pro-inflammatory genes, including c-Rel, O×40 and IL-2. Regnase-1 activation is transient and is subject to negative feedback mechanisms including proteasome-mediated degradation or mucosa-associated lymphoid tissue 1 (MALT1) mediated cleavage. The major function of Regnase-1 is promoting mRNA decay via its ribonuclease activity by specifically targeting a subset of genes in different cell types. In monocytes, Regnase-1 downregulates IL-6 and IL-12B mRNAs, thus mitigating inflammation, whereas in T cells, it restricts T-cell activation by targeting c-Rel, 0×40 and IL-2 transcripts. In cancer cells, Regnase-1 promotes apoptosis by inhibiting anti-apoptotic genes including Bcl2L1, Bcl2A1, RelB and Bcl3.

In some embodiments, the modified TILs described herein comprise reduced expression and/or function of the CBLB gene. This gene encodes CBL-B, also referred to as RNF56, Nbla00127 and Cbl proto-oncogene B. CBL-B is an E3 ubiquitin-protein ligase and a member of the CBL gene family. CBL-B functions as a negative regulator of T-cell activation. CBL-B expression in T cells causes ligand-induced T cell receptor down-modulation, controlling the activation degree of T cells during antigen presentation. Mutation of the CBLB gene has been associated with autoimmune conditions such as type 1 diabetes.

In some embodiments, the modified TILs described herein comprise reduced expression and/or function of the RC3H1 gene. This gene encodes Ring finger and CCCH-type domains 1, also referred to as Roquin-1. Roquin-1 recognizes and binds to a constitutive decay element (CDE) in the 3′ UTR of mRNAs, leading to mRNA deadenylation and degradation. Alternative splicing results in multiple transcript variants.

In some embodiments, the modified TILs described herein comprise reduced expression and/or function of the NFKBIA gene. This gene encodes IκBα, also referred to as NFKB inhibitor alpha, MAD-3, NFKBI and EDAID2. IκBα is one member of a family of cellular proteins that function to inhibit the NF-κB transcription factor. IκBα inhibits NF-κB by masking the nuclear localization signals (NLS) of NF-κB proteins and keeping them sequestered in an inactive state in the cytoplasm. In addition, IκBα blocks the ability of NF-κB transcription factors to bind to DNA, which is required for NF-κB's proper functioning. The NFKBIA gene is mutated in some Hodgkin's lymphoma cells; such mutations inactivate the IκBα protein, thus causing NF-κB to be chronically active in the lymphoma tumor cells and this activity contributes to the malignant state of these tumor cells.

TABLE 3
Endogenous target genes

| | | Human | Human | Murine | Murine |
|---|---|---|---|---|---|
| Gene | | UniProt | NCBI | UniProt | NCBI |
| Symbol | Gene Name | Ref. | Ref | Ref. | Ref |
| SOCS1 | suppressor of | O15524 | 8651 | O35716 | 12703 |
| | cytokine | | (SEQ ID | | (SEQ ID |
| | signaling 1 | | NO: 1) | | NO: 2) |
| PTPN2 | protein tyrosine | P17706 | 5771 | Q06180 | 19255 |
| | phosphatase, non- | | (SEQ ID | | (SEQ ID |
| | receptor type 2 | | NO: 3) | | NO: 4) |
| ZC3H12A | Endoribonuclease | Q5D1E8 | 80149 | Q5D1E7 | 230738 |
| | ZC3H12A | | (SEQ ID | | (SEQ ID |
| | | | NO: 5) | | NO: 6) |
| CBLB | Cbl proto- | Q13191 | 868 | Q3TTA7 | 208650 |
| | oncogene B | | (SEQ ID | | (SEQ ID |
| | | | NO: 7) | | NO: 8) |
| RC3H1 | Ring finger and | Q5TC82 | 149041 | Q4VGL6 | 381305 |
| | CCCH-type | | (SEQ ID | | (SEQ ID |
| | domains 1 | | NO: 9) | | NO: 10) |
| NFKBIA | NFKB inhibitor | P25963 | 4792 | Q9Z1E3 | 18035 |
| | alpha | | (SEQ ID | | (SEQ ID |
| | | | NO: 11) | | NO: 12) |

In some embodiments, the modified TILs comprise reduced expression and/or function of any one or two or more of SOCS1, PTPN2, ZC3H12A, CBLB, RC3H1 or NFKBIA. In some embodiments, the modified TILs comprise reduced expression and/or function of at least one endogenous target gene selected from SOCS1, PTPN2, ZC3H12A, RC3H1 and NFKBIA and further comprise reduced expression and/or function of CBLB. In some embodiments, the modified TILs comprise reduced expression and/or function of at least two endogenous target genes selected from SOCS, PTPN2, ZC3H12A, RC3H1 and NFKBIA and further comprise reduced expression and/or function of CBLB.

In some embodiments, the modified TILs comprise reduced expression and/or function of at least one endogenous target gene selected from CBLB, PTPN2, ZC3H12A, RC3H1 and NFKBIA and further comprise reduced expression and/or function of SOCS. In some embodiments, the modified TILs comprise reduced expression and/or function of at least two endogenous target genes selected from CBLB, PTPN2, ZC3H12A, RC3H1 and NFKBIA and further comprise reduced expression and/or function of SOCS1.

In some embodiments, the modified TILs comprise reduced expression and/or function of at least one endogenous target gene selected from CBLB, SOCS1, ZC3H12A, RC3H1 and NFKBIA and further comprise reduced expression and/or function of PTPN2. In some embodiments, the modified TILs comprise reduced expression and/or function of at least two endogenous target genes selected from CBLB, SOCS1, ZC3H12A, RC3H1 and NFKBIA and further comprise reduced expression and/or function of PTPN2.

In some embodiments, the modified TILs comprise reduced expression and/or function of at least one endogenous target gene selected from CBLB, SOCS1, PTPN2, RC3H1 and NFKBIA and further comprise reduced expression and/or function of ZC3H12A. In some embodiments, the modified TILs comprise reduced expression and/or function of at least two endogenous target genes selected from CBLB, SOCS1, PTPN2, RC3H1 and NFKBIA and further comprise reduced expression and/or function of ZC3H12A.

In some embodiments, the modified TILs comprise reduced expression and/or function of at least one endogenous target gene selected from CBLB, SOCS1, PTPN2, ZC3H12A and NFKBIA and further comprise reduced expression and/or function of RC3H1. In some embodiments, the modified TILs comprise reduced expression and/or function of at least two endogenous target genes selected from CBLB, SOCS1, PTPN2, ZC3H12A and NFKBIA and further comprise reduced expression and/or function of RC3H1.

In some embodiments, the modified TILs comprise reduced expression and/or function of at least one endogenous target gene selected from CBLB, SOCS1, PTPN2, ZC3H12A and RC3H1 and further comprise reduced expression and/or function of NFKBIA. In some embodiments, the modified TILs comprise reduced expression and/or function of at least two endogenous target genes selected from CBLB, SOCS1, PTPN2, ZC3H12A and RC3H1 and further comprise reduced expression and/or function of NFKBIA.

### II. Gene-Regulating Systems

Herein, the term “gene-regulating system” refers to a protein, nucleic acid, or combination thereof that is capable of modifying an endogenous target DNA sequence when introduced into a cell, thereby regulating the expression or function of the encoded gene product. Numerous gene-regulating systems suitable for use in the methods of the present disclosure are known in the art including, but not limited to, shRNAs, siRNAs, zinc-finger nuclease systems, TALEN systems, and CRISPR/Cas systems. In some embodiments, the gene-regulating system is a gene-editing system. Gene editing systems suitable for use in the methods of the present disclosure are known in the art including, but not limited to, zinc-finger nuclease systems, TALEN systems, and CRISPR/Cas systems.

As used herein, “regulate,” when used in reference to the effect of a gene-regulating system on an endogenous target gene encompasses any change in the sequence of the endogenous target gene, any change in the epigenetic state of the endogenous target gene, and/or any change in the expression or function of the protein encoded by the endogenous target gene.

In some embodiments, the gene-regulating system may mediate a change in the sequence of the endogenous target gene, for example, by introducing one or more mutations into the endogenous target sequence, such as by insertion or deletion of one or more nucleic acids in the endogenous target sequence. Exemplary mechanisms that can mediate alterations of the endogenous target sequence include, but are not limited to, non-homologous end joining (NHEJ) (e.g., classical or alternative), microhomology-mediated end joining (MMEJ), homology-directed repair (e.g., endogenous donor template mediated), SDSA (synthesis dependent strand annealing), single strand annealing or single strand invasion.

In some embodiments, the gene-regulating system may mediate a change in the epigenetic state of the endogenous target sequence. For example, in some embodiments, the gene-regulating system may mediate covalent modifications of the endogenous target gene DNA (e.g., cytosine methylation and hydroxymethylation) or of associated histone proteins (e.g. lysine acetylation, lysine and arginine methylation, serine and threonine phosphorylation, and lysine ubiquitination and sumoylation).

In some embodiments, the gene-regulating system may mediate a change in the expression of the protein encoded by the endogenous target gene. In such embodiments, the gene-regulating system may regulate the expression of the encoded protein by modifications of the endogenous target DNA sequence, or by acting on the mRNA product encoded by the DNA sequence. In some embodiments, the gene-regulating system may result in the expression of a modified endogenous protein. In such embodiments, the modifications to the endogenous DNA sequence mediated by the gene-regulating system result in the expression of an endogenous protein demonstrating a reduced function as compared to the corresponding endogenous protein in an unmodified TIL. In such embodiments, the expression level of the modified endogenous protein may be increased, decreased or may be the same, or substantially similar to, the expression level of the corresponding endogenous protein in an unmodified immune cell.

### A. Nucleic Acid-Based Gene-Regulating Systems

In some embodiments, the present disclosure provides nucleic acid gene-regulating systems comprising one, two or more nucleic acids capable of reducing the expression and/or function of at least one, two, or more endogenous gene selected from ANKRD11, BCL2L11, BCL3, BCOR, CALM2, CBLB, CHIC2, CTLA4, DHODH, E2F8, EGR2, FLI1, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.) In some embodiments, the present disclosure provides nucleic acid gene-regulating systems comprising one, two or more nucleic acids capable of reducing the expression and/or function of at least one endogenous gene selected from SOCS1, PTPN2, ZC3H12A, CBLB, RC3H1 and NFKBIA. In some embodiments, the present disclosure provides nucleic acid gene-regulating systems comprising nucleic acids capable of reducing the expression and/or function of SOCS1 and at least one, two or more endogenous target genes selected from PTPN2, ZC3H12A, CBLB, RC3H, and NFKBIA. In some embodiments, the present disclosure provides modified TILs manufactured by the methods described herein comprising such gene-regulating systems. As used herein, a nucleic acid-based gene-regulating system is a system comprising one or more nucleic acid molecules that is capable of regulating the expression of an endogenous target gene without the requirement for an exogenous protein. In some embodiments, the gene-regulating system comprises an RNA interference molecule or antisense RNA molecule that is complementary to a target nucleic acid sequence.

An “antisense RNA molecule” refers to an RNA molecule, regardless of length, that is complementary to an mRNA transcript. Antisense RNA molecules refer to single stranded RNA molecules that can be introduced to a cell, tissue, or subject and result in decreased expression of an endogenous target gene product through mechanisms that do not rely on endogenous gene silencing pathways, but rather rely on RNaseH-mediated degradation of the target mRNA transcript. In some embodiments, an antisense nucleic acid comprises a modified backbone, for example, phosphorothioate, phosphorodithioate, or others known in the art, or may comprise non-natural internucleoside linkages. In some embodiments, an antisense nucleic acid can comprise locked nucleic acids (LNA).

“RNA interference molecule” as used herein refers to an RNA polynucleotide that mediates the decreased the expression of an endogenous target gene product by degradation of a target mRNA through endogenous gene silencing pathways (e.g., Dicer and RNA-induced silencing complex (RISC)). Exemplary RNA interference agents include micro RNAs (also referred to herein as “miRNAs”), short hair-pin RNAs (shRNAs), small interfering RNAs (siRNAs), RNA aptamers, and morpholinos.

In some embodiments, the gene-regulating system comprises one or more miRNAs. miRNAs are naturally occurring, small non-coding RNA molecules of about 21-25 nucleotides in length. miRNAs are at least partially complementary to one or more target mRNA molecules. miRNAs can downregulate (e.g., decrease) expression of an endogenous target gene product through translational repression, cleavage of the mRNA, and/or deadenylation.

In some embodiments, the gene-regulating system comprises one or more shRNAs. shRNAs are single stranded RNA molecules of about 50-70 nucleotides in length that form stem-loop structures and result in degradation of complementary mRNA sequences. shRNAs can be cloned in plasmids or in non-replicating recombinant viral vectors to be introduced intracellularly and result in the integration of the shRNA-encoding sequence into the genome. As such, an shRNA can provide stable and consistent repression of endogenous target gene translation and expression.

In some embodiments, nucleic acid-based gene-regulating system comprises one or more siRNAs. siRNAs refer to double stranded RNA molecules typically about 21-23 nucleotides in length. The siRNA associates with a multi protein complex called the RNA-induced silencing complex (RISC), during which the “passenger” sense strand is enzymatically cleaved. The antisense “guide” strand contained in the activated RISC then guides the RISC to the corresponding mRNA because of sequence homology and the same nuclease cuts the target mRNA, resulting in specific gene silencing. Optimally, an siRNA is 18, 19, 20, 21, 22, 23 or 24 nucleotides in length and has a 2 base overhang at its 3′ end. siRNAs can be introduced to an individual cell and/or culture system and result in the degradation of target mRNA sequences. siRNAs and shRNAs are further described in Fire et al., Nature, 391:19, 1998 and U.S. Pat. Nos. 7,732,417; 8,202,846; and 8,383,599.

In some embodiments, the gene-regulating system comprises one or more morpholinos. “Morpholino” as used herein refers to a modified nucleic acid oligomer wherein standard nucleic acid bases are bound to morpholine rings and are linked through phosphorodiamidate linkages. Similar to siRNA and shRNA, morpholinos bind to complementary mRNA sequences. However, morpholinos function through steric-inhibition of mRNA translation and alteration of mRNA splicing rather than targeting complementary mRNA sequences for degradation.

In some embodiments, the gene-regulating system comprises a nucleic acid molecule that binds to a target RNA sequence that is at least 90% identical to a RNA sequence encoded by a DNA sequence defined by a set of genomic coordinates shown in Tables 4, -5, 9-12, and 17-22. Throughout this application, the referenced genomic coordinates are based on genomic annotations in the GRCh38 (also referred to as hg38) assembly of the human genome from the Genome Reference Consortium, available at the National Center for Biotechnology Information website. Tools and methods for converting genomic coordinates between one assembly and another are known in the art and can be used to convert the genomic coordinates provided herein to the corresponding coordinates in another assembly of the human genome, including conversion to an earlier assembly generated by the same institution or using the same algorithm (e.g., from GRCh38 to GRCh37), and conversion an assembly generated by a different institution or algorithm (e.g., from GRCh38 to NCBI33, generated by the International Human Genome Sequencing Consortium). Available methods and tools known in the art include, but are not limited to, NCBI Genome Remapping Service, available at the National Center for Biotechnology Information website, UCSC LiftOver, available at the UCSC Genome Brower website, and Assembly Converter, available at the Ensembl.org website.

In some embodiments, the nucleic acid-based gene-regulating system comprises at least one nucleic acid molecule (e.g., an siRNA, an shRNA, an RNA aptamer, or a morpholino), wherein the at least one nucleic acid molecule is a SOCS1-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2). In some embodiments, the at least one SOCS1-targeting nucleic acid molecule is an siRNA or an shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 4 (human genome) or Table 5 (mouse genome). In some embodiments, the at least one SOCS1-targeting nucleic acid molecule binds to a target RNA sequence that is 100% identical to a RNA sequence encoded by a DNA sequence defined by a set of genomic coordinates shown in Table 4 or Table 5. In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 23-200. In some embodiments, the at least one SOCS1-targeting nucleic acid molecule binds to a human target RNA sequence that is at least 95%, 96%, 97%, 98%, or 99% identical to a human RNA sequence encoded by one of SEQ ID NOs: 23-35 and 56-187. In some embodiments, the at least one SOCS1-targeting nucleic acid molecule binds to a target RNA sequence that is 100% identical to an RNA sequence encoded by one of SEQ ID NOs: 23-200. In some embodiments, the at least one SOCS1-targeting nucleic acid molecule binds to a target human RNA sequence that is 100% identical to a human RNA sequence encoded by one of SEQ ID NOs: 23-35 and 56-187.

In some embodiments, the at least one SOCS1-targeting nucleic acid molecule is a SOCS1-targeting shRNA or siRNA molecule. In some embodiments, the at least one SOCS1-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 4 or Table 5. In some embodiments, the at least one SOCS1-targeting shRNA or siRNA molecule binds to a target RNA sequence that is 100% identical to a RNA sequence encoded by a DNA sequence defined by a set of genomic coordinates shown in Table 4 or Table 5. In some embodiments, the at least one SOCS1-targeting shRNA or siRNA molecule binds to a target RNA sequence that is at least 95%, 96%, 97%, 98%, or 99% identical to an RNA sequence encoded by one of SEQ ID NOs: 23-55 or 23-200. In some embodiments, the at least one SOCS1-targeting shRNA or siRNA molecule binds to a target human RNA sequence that is at least 95%, 96%, 97%, 98%, or 99% identical to a human RNA sequence encoded by one of SEQ ID NOs: 23-35 and 56-187. In some embodiments, the at least one SOCS1-targeting shRNA or siRNA molecule binds to a target RNA sequence that is 100% identical to an RNA sequence encoded by one of SEQ ID NOs: 23-55 or 23-200. In some embodiments, the at least one SOCS1-targeting shRNA or siRNA molecule binds to a target human RNA sequence that is 100% identical to a human RNA sequence encoded by one of SEQ ID NOs: 23-35 and 56-187.

In some embodiments, the nucleic acid-based gene-regulating system comprises at least one SOCS1-targeting siRNA molecule or shRNA molecule selected from those known in the art. For example, in some embodiments, the SOCS1-targeting nucleic acid molecule is a SOCS1-targeting siRNA comprising a nucleic acid sequence selected from SEQ ID NOs: 13-22. (See International PCT Publication Nos. WO 2017120996; WO 2018137295; WO 2017120998; and WO 2018137293, incorporated by reference herein in their entireties) (Table 6). In some embodiments, the SOCS-targeting siRNA molecule or shRNA molecule is encoded by a nucleic acid sequence selected from SEQ ID NOs: 13-200. In some embodiments, the SOCS-targeting siRNA molecule or shRNA molecule is encoded by a human nucleic acid sequence selected from SEQ ID NOs: 23-35 and 56-187. In some embodiments, the SOCS1-targeting nucleic acid molecule is a SOCS1-targeting shRNA molecule or siRNA molecule that binds to a human target sequence selected from SEQ ID NOs: 23-35 (See U.S. Pat. No. 8,324,369, incorporated herein by reference in its entirety. (Table 7). In some embodiments, the SOCS1-targeting nucleic acid molecule is a SOCS-targeting shRNA molecule or siRNA molecule that binds to a mouse target sequence selected from SEQ ID NOs: 36-55 (See U.S. Pat. No. 9,944,931, incorporated by reference herein in its entirety) (Table 8).

TABLE 4
SOCS1 Human Genome Coordinates

| Target | Coordinates |
|---|---|
| SOCS1 | chr16: 11255187-11255206 |
| SOCS1 | chr16: 11255238-11255257 |
| SOCS1 | chr16: 11255058-11255077 |
| SOCS1 | chr16: 11255158-11255177 |
| SOCS1 | chr16: 11255239-11255258 |
| SOCS1 | chr16: 11255237-11255256 |
| SOCS1 | chr16: 11255019-11255038 |
| SOCS1 | chr16: 11255066-11255085 |
| SOCS1 | chr16: 11255238-11255257 |
| SOCS1 | chr16: 11255168-11255187 |
| SOCS1 | chr16: 11255079-11255098 |
| SOCS1 | chr16: 11255287-11255306 |
| SOCS1 | chr16: 11255249-11255268 |
| SOCS1 | chr16: 11255186-11255205 |
| SOCS1 | chr16: 11255236-11255255 |
| SOCS1 | chr16: 11255116-11255135 |
| SOCS1 | chr16: 11255070-11255089 |
| SOCS1 | chr16: 11255117-11255136 |
| SOCS1 | chr16: 11255283-11255302 |
| SOCS1 | chr16: 11255442-11255461 |
| SOCS1 | chr16: 11255209-11255228 |
| SOCS1 | chr16: 11254932-11254951 |
| SOCS1 | chr16: 11254966-11254985 |
| SOCS1 | chr16: 11254950-11254969 |
| SOCS1 | chr16: 11255049-11255068 |
| SOCS1 | chr16: 11255155-11255174 |
| SOCS1 | chr16: 11255460-11255479 |
| SOCS1 | chr16: 11255037-11255056 |
| SOCS1 | chr16: 11255154-11255173 |
| SOCS1 | chr16: 11255115-11255134 |
| SOCS1 | chr16: 11254985-11255004 |
| SOCS1 | chr16: 11255013-11255032 |
| SOCS1 | chr16: 11255016-11255035 |
| SOCS1 | chr16: 11255139-11255158 |
| SOCS1 | chr16: 11255248-11255267 |
| SOCS1 | chr16: 11255217-11255236 |
| SOCS1 | chr16: 11254994-11255013 |
| SOCS1 | chr16: 11254965-11254984 |
| SOCS1 | chr16: 11255219-11255238 |
| SOCS1 | chr16: 11255173-11255192 |
| SOCS1 | chr16: 11255210-11255229 |
| SOCS1 | chr16: 11255062-11255081 |
| SOCS1 | chr16: 11255259-11255278 |
| SOCS1 | chr16: 11255230-11255249 |
| SOCS1 | chr16: 11255084-11255103 |
| SOCS1 | chr16: 11255175-11255194 |
| SOCS1 | chr16: 11255419-11255438 |
| SOCS1 | chr16: 11254903-11254922 |
| SOCS1 | chr16: 11255089-11255108 |
| SOCS1 | chr16: 11255379-11255398 |
| SOCS1 | chr16: 11255206-11255225 |
| SOCS1 | chr16: 11255090-11255109 |
| SOCS1 | chr16: 11255208-11255227 |
| SOCS1 | chr16: 11254956-11254975 |
| SOCS1 | chr16: 11255118-11255137 |
| SOCS1 | chr16: 11254906-11254925 |
| SOCS1 | chr16: 11255167-11255186 |
| SOCS1 | chr16: 11254835-11254854 |
| SOCS1 | chr16: 11255292-11255311 |
| SOCS1 | chr16: 11255416-11255435 |
| SOCS1 | chr16: 11255136-11255155 |
| SOCS1 | chr16: 11254964-11254983 |
| SOCS1 | chr16: 11254896-11254915 |
| SOCS1 | chr16: 11254940-11254959 |
| SOCS1 | chr16: 11255349-11255368 |
| SOCS1 | chr16: 11254992-11255011 |
| SOCS1 | chr16: 11254923-11254942 |
| SOCS1 | chr16: 11255431-11255450 |
| SOCS1 | chr16: 11255463-11255482 |
| SOCS1 | chr16: 11255343-11255362 |
| SOCS1 | chr16: 11255088-11255107 |
| SOCS1 | chr16: 11254834-11254853 |
| SOCS1 | chr16: 11254922-11254941 |
| SOCS1 | chr16: 11255098-11255117 |
| SOCS1 | chr16: 11254993-11255012 |
| SOCS1 | chr16: 11254840-11254859 |
| SOCS1 | chr16: 11255400-11255419 |
| SOCS1 | chr16: 11254920-11254939 |
| SOCS1 | chr16: 11254966-11254985 |
| SOCS1 | chr16: 11254860-11254879 |
| SOCS1 | chr16: 11254980-11254999 |
| SOCS1 | chr16: 11254857-11254876 |
| SOCS1 | chr16: 11254874-11254893 |
| SOCS1 | chr16: 11255028-11255047 |
| SOCS1 | chr16: 11254956-11254975 |
| SOCS1 | chr16: 11254908-11254927 |
| SOCS1 | chr16: 11255337-11255356 |
| SOCS1 | chr16: 11254836-11254855 |
| SOCS1 | chr16: 11254842-11254861 |
| SOCS1 | chr16: 11254865-11254884 |
| SOCS1 | chr16: 11254830-11254849 |
| SOCS1 | chr16: 11255401-11255420 |
| SOCS1 | chr16: 11254864-11254883 |
| SOCS1 | chr16: 11255311-11255330 |
| SOCS1 | chr16: 11255343-11255362 |
| SOCS1 | chr16: 11255342-11255361 |
| SOCS1 | chr16: 11255272-11255291 |
| SOCS1 | chr16: 11254866-11254885 |
| SOCS1 | chr16: 11255310-11255329 |
| SOCS1 | chr16: 11255336-11255355 |
| SOCS1 | chr16: 11255416-11255435 |
| SOCS1 | chr16: 11255402-11255421 |
| SOCS1 | chr16: 11255467-11255486 |
| SOCS1 | chr16: 11254873-11254892 |
| SOCS1 | chr16: 11255265-11255284 |
| SOCS1 | chr16: 11254820-11254839 |
| SOCS1 | chr16: 11254848-11254867 |
| SOCS1 | chr16: 11255317-11255336 |
| SOCS1 | chr16: 11255351-11255370 |
| SOCS1 | chr16: 11254811-11254830 |
| SOCS1 | chr16: 11255353-11255372 |
| SOCS1 | chr16: 11255350-11255369 |
| SOCS1 | chr16: 11255309-11255328 |
| SOCS1 | chr16: 11255390-11255409 |
| SOCS1 | chr16: 11255478-11255497 |
| SOCS1 | chr16: 11255330-11255349 |
| SOCS1 | chr16: 11254875-11254894 |
| SOCS1 | chr16: 11255124-11255143 |
| SOCS1 | chr16: 11255352-11255371 |
| SOCS1 | chr16: 11254872-11254891 |
| SOCS1 | chr16: 11255331-11255350 |
| SOCS1 | chr16: 11255315-11255334 |
| SOCS1 | chr16: 11255482-11255501 |
| SOCS1 | chr16: 11254995-11255014 |
| SOCS1 | chr16: 11255316-11255335 |
| SOCS1 | chr16: 11255308-11255327 |
| SOCS1 | chr16: 11255321-11255340 |
| SOCS1 | chr16: 11255322-11255341 |
| SOCS1 | chr16: 11255330-11255349 |
| SOCS1 | chr16: 11255368-11255387 |
| SOCS1 | chr16: 11255377-11255396 |
| SOCS1 | chr16: 11255380-11255399 |

TABLE 5
Socs1 Murine Genome Coordinates

| Target | Coordinates |
|---|---|
| Socs1 | chr16: 10784479-10784498 |
| Socs1 | chr16: 10784409-10784428 |
| Socs1 | chr16: 10784456-10784475 |
| Socs1 | chr16: 10784322-10784341 |
| Socs1 | chr16: 10784548-10784567 |
| Socs1 | chr16: 10784596-10784615 |
| Socs1 | chr16: 10784264-10784283 |
| Socs1 | chr16: 10784628-10784647 |
| Socs1 | chr16: 10784526-10784545 |
| Socs1 | chr16: 10784508-10784527 |
| Socs1 | chr16: 10784565-10784584 |
| Socs1 | chr16: 10784474-10784493 |
| Socs1 | chr16: 10784293-10784312 |

TABLE 6
Exemplary human SOCSI siRNAs

| | | SEQ |
|---|---|---|
| Target | Sequence | ID |
| SOCS1_siRNA_1 | CGCACUUCCGCACAUUCCGUUCG | 13 |
| SOCS1_siRNA_2 | GGGGAGGGUCUCUGGCUUUAUUU | 14 |
| SOCS1_siRNA_3 | CAGCAUUAACUGGGAUGCCGUGU | 15 |
| SOCS1_siRNA_4 | CCAGGACCUGAACUCGCACCUCC | 16 |
| SOCS1_siRNA_5 | UACAUAUACCCAGUAUCUUUGCA | 17 |
| SOCS1_siRNA_6 | GCCGACAAUGCAGUCUCCACAGC | 18 |
| SOCS1_siRNA_7 | CCCCUGGUUGUUGUAGCAGCUUA | 19 |
| SOCS1_siRNA_8 | CUGCUGUGCAGAAUCCUAUUUUA | 20 |
| SOCS1_siRNA_9 | UGGGAUGCCGUGUUAUUUUGUUA | 21 |
| SOCS1_siRNA_10 | UCGCACCUCCUACCUCUUCAUGU | 22 |

TABLE 7
Exemplary human SOCSI shRNA and
siRNA target sequences

| | | SEQ |
|---|---|---|
| Target | Sequence | ID |
| SOCS1_shRNA_1 | CACGCACTTCCGCACATTC | 23 |
| SOCS1_shRNA_2 | TTCCGTTCGCACGCCGATT | 24 |
| SOCS1_shRNA_3 | GAGCTTCGACTGCCTCTTC | 25 |
| SOCS1_siRNA_1 | CGCACTTCCGCACATTCCGTTCG | 26 |
| SOCS1_siRNA_2 | GGGGAGGGTCTCTGGCTTTATTT | 27 |
| SOCS1_siRNA_3 | CAGCATTAACTGGGATGCCGTGT | 28 |
| SOCS1_siRNA_4 | CCAGGACCTGAACTCGCACCTCC | 29 |
| SOCS1_siRNA_5 | TACATATACCCAGTATCTTTGCA | 30 |
| SOCS1_siRNA_6 | GCCGACAATGCAGTCTCCACAGC | 31 |
| SOCS1_siRNA_7 | CCCCTGGTTGTTGTAGCAGCTTA | 32 |
| SOCS1_siRNA_8 | CTGCTGTGCAGAATCCTATTTTA | 33 |
| SOCS1_siRNA_9 | TGGGATGCCGTGTTATTTTGTTA | 34 |
| SOCS1_siRNA_10 | TCGCACCTCCTACCTCTTCATGT | 35 |

TABLE 8
Exemplary murine Socs1 shRNA and
siRNA target sequences

| | | SEQ |
|---|---|---|
| Target | Sequence | ID |
| ND000214 | TTTCGAGCTGCTGGAGCACTA | 36 |
| ND000219 | TCGAGCTGCTGGAGCACTACG | 37 |
| TRCN0000231240 | TCGCCAACGGAACTGCTTCTT | 38 |
| ND000218 | ACTTCTGGCTGGAGACCTCAT | 39 |
| TRCN0000067420 | GCGAGACCTTCGACTGCCTTT | 40 |
| TRCN0000067418 | CGACACTCACTTCCGCACCTT | 41 |
| ND000220 | CTACCTGAGTTCCTTCCCCTT | 42 |
| TRCN0000231238 | TTCCGCTCCCACTCCGATTAC | 43 |
| TRCN0000231241 | TAACCCGGTACTCCGTGACTA | 44 |
| ND000216 | TACTCCGTGACTACCTGAGTT | 45 |
| ND000211 | CTTCCGCTCCCACTCCGATTA | 46 |
| TRCN0000067422 | GCGCGACAGTCGCCAACGGAA | 47 |
| TRCN0000231239 | TGGACGCCTGCGGCTTCTATT | 48 |
| TRCN0000067419 | CGCATCCCTCTTAACCCGGTA | 49 |
| ND000212 | TACATATTCCCAGTATCTTTG | 50 |
| TRCN0000231242 | GCGCCTTATTATTTCTTATTA | 51 |
| TRCN0000067421 | CCGTGACTACCTGAGTTCCTT | 52 |
| ND000215 | GGAGGGTCTCTGGCTTCATTT | 53 |
| ND000213 | TTCGCGCTCAGCGTGAAGATG | 54 |
| ND000217 | ATCCCTCTTAACCCGGTACTC | 55 |

In some embodiments, the nucleic acid-based gene-regulating system comprises at least one nucleic acid molecule (e.g., an siRNA, an shRNA, an RNA aptamer, or a morpholino), wherein the at least one nucleic acid molecule is a PTPN2-targeting nucleic acid molecule. In some embodiments, the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4). In some embodiments, the at least one PTPN2-targeting nucleic acid molecule is an siRNA or an shRNA molecule. In some embodiments, the at least one PTPN2-targeting siRNA or an shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one PTPN2-targeting siRNA or an shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 9 (human genome) or Table 10 (mouse genome). In some embodiments, the at least one PTPN2-targeting nucleic acid molecule binds to a target RNA sequence that is 100% identical to a RNA sequence encoded by a DNA sequence defined by a set of genomic coordinates shown in Table 9 or Table 10. In some embodiments, the at least one PTPN2-targeting nucleic acid molecule binds to a target RNA sequence that is at least 95%, 96%, 97%, 98%, or 99% identical to an RNA sequence encoded by one of SEQ ID NOs: 201-327. In some embodiments, the at least one PTPN2-targeting nucleic acid molecule binds to a human target RNA sequence that is at least 95%, 96%, 97%, 98%, or 99% identical to a human RNA sequence encoded by one of SEQ ID NOs: 201-314. In some embodiments, the at least one PTPN2-targeting nucleic acid molecule binds to a target RNA sequence that is 100% identical to an RNA sequence encoded by one of SEQ ID NOs: 201-327. In some embodiments, the at least one PTPN2-targeting nucleic acid molecule binds to a human target RNA sequence that is 100% identical to a human RNA sequence encoded by one of SEQ ID NOs: 201-314.

In some embodiments, the at least one PTPN2-targeting nucleic acid molecule is a SOCS1-targeting shRNA or siRNA molecule. In some embodiments, the at least one PTPN2-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one PTPN2-targeting shRNA or siRNA molecule binds to a target RNA sequence that is 100% identical to a RNA sequence encoded by a DNA sequence defined by a set of genomic coordinates shown in Table 9 or Table 10. In some embodiments, the at least one PTPN2-targeting shRNA or siRNA molecule binds to a target RNA sequence that is at least 95%, 96%, 97%, 98%, or 99% identical to an RNA sequence encoded by one of SEQ ID NOs: 201-327. In some embodiments, the at least one PTPN2-targeting shRNA or siRNA molecule binds to a human target RNA sequence that is at least 95%, 96%, 97%, 98%, or 99% identical to a human RNA sequence encoded by one of SEQ ID NOs: 201-314. In some embodiments, the at least one PTPN2-targeting shRNA or siRNA molecule binds to a human target RNA sequence that is 100% identical to a human RNA sequence encoded by one of SEQ ID NOs: 201-327. In some embodiments, the at least one PTPN2-targeting shRNA or siRNA molecule binds to a target RNA sequence that is 100% identical to an RNA sequence encoded by one of SEQ ID NOs: 201-314.

TABLE 9
PTPN2 Human Genome Coordinates

| Target | Coordinates |
|---|---|
| PTPN2 | Chr18: 12859218-12859237 |
| PTPN2 | Chr18: 12884109-12884128 |
| PTPN2 | Chr18: 12817227-12817246 |
| PTPN2 | Chr18: 12817234-12817253 |
| PTPN2 | Chr18: 12884091-12884110 |
| PTPN2 | Chr18: 12884121-12884140 |
| PTPN2 | Chr18: 12831010-12831029 |
| PTPN2 | Chr18: 12817208-12817227 |
| PTPN2 | Chr18: 12817158-12817177 |
| PTPN2 | Chr18: 12831016-12831035 |
| PTPN2 | Chr18: 12817228-12817247 |
| PTPN2 | Chr18: 12830964-12830983 |
| PTPN2 | Chr18: 12801972-12801991 |
| PTPN2 | Chr18: 12836818-12836837 |
| PTPN2 | Chr18: 12817215-12817234 |
| PTPN2 | Chr18: 12802018-12802037 |
| PTPN2 | Chr18: 12884116-12884135 |
| PTPN2 | Chr18: 12840739-12840758 |
| PTPN2 | Chr18: 12802004-12802023 |
| PTPN2 | Chr18: 12840767-12840786 |
| PTPN2 | Chr18: 12817197-12817216 |
| PTPN2 | Chr18: 12884108-12884127 |
| PTPN2 | Chr18: 12817221-12817240 |
| PTPN2 | Chr18: 12836820-12836839 |
| PTPN2 | Chr18: 12884124-12884143 |
| PTPN2 | Chr18: 12830996-12831015 |
| PTPN2 | Chr18: 12830942-12830961 |
| PTPN2 | Chr18: 12884112-12884131 |
| PTPN2 | Chr18: 12817193-12817212 |
| PTPN2 | Chr18: 12859205-12859224 |
| PTPN2 | Chr18: 12817202-12817221 |
| PTPN2 | Chr18: 12859216-12859235 |
| PTPN2 | Chr18: 12859215-12859234 |
| PTPN2 | Chr18: 12817201-12817220 |
| PTPN2 | Chr18: 12802134-12802153 |
| PTPN2 | Chr18: 12884075-12884094 |
| PTPN2 | Chr18: 12884115-12884134 |
| PTPN2 | Chr18: 12840757-12840776 |
| PTPN2 | Chr18: 12814205-12814224 |
| PTPN2 | Chr18: 12840777-12840796 |
| PTPN2 | Chr18: 12814277-12814296 |
| PTPN2 | Chr18: 12840746-12840765 |
| PTPN2 | Chr18: 12801989-12802008 |
| PTPN2 | Chr18: 12819237-12819256 |
| PTPN2 | Chr18: 12814348-12814367 |
| PTPN2 | Chr18: 12794428-12794447 |
| PTPN2 | Chr18: 12831005-12831024 |
| PTPN2 | Chr18: 12825890-12825909 |
| PTPN2 | Chr18: 12840723-12840742 |
| PTPN2 | Chr18: 12840747-12840766 |
| PTPN2 | Chr18: 12802068-12802087 |
| PTPN2 | Chr18: 12840716-12840735 |
| PTPN2 | Chr18: 12840773-12840792 |
| PTPN2 | Chr18: 12831012-12831031 |
| PTPN2 | Chr18: 12814240-12814259 |
| PTPN2 | Chr18: 12802130-12802149 |
| PTPN2 | Chr18: 12794454-12794473 |
| PTPN2 | Chr18: 12817208-12817227 |
| PTPN2 | Chr18: 12819226-12819245 |
| PTPN2 | Chr18: 12825889-12825908 |
| PTPN2 | Chr18: 12840782-12840801 |
| PTPN2 | Chr18: 12836812-12836831 |
| PTPN2 | Chr18: 12817298-12817317 |
| PTPN2 | Chr18: 12817324-12817343 |
| PTPN2 | Chr18: 12819268-12819287 |
| PTPN2 | Chr18: 12817303-12817322 |
| PTPN2 | Chr18: 12825927-12825946 |
| PTPN2 | Chr18: 12817220-12817239 |
| PTPN2 | Chr18: 12825901-12825920 |
| PTPN2 | Chr18: 12814222-12814241 |
| PTPN2 | Chr18: 12831000-12831019 |
| PTPN2 | Chr18: 12840738-12840757 |
| PTPN2 | Chr18: 12802057-12802076 |
| PTPN2 | Chr18: 12802069-12802088 |
| PTPN2 | Chr18: 12884123-12884142 |
| PTPN2 | Chr18: 12814294-12814313 |
| PTPN2 | Chr18: 12817283-12817302 |
| PTPN2 | Chr18: 12830945-12830964 |
| PTPN2 | Chr18: 12817284-12817303 |
| PTPN2 | Chr18: 12817256-12817275 |
| PTPN2 | Chr18: 12884062-12884081 |
| PTPN2 | Chr18: 12814295-12814314 |
| PTPN2 | Chr18: 12817313-12817332 |
| PTPN2 | Chr18: 12814255-12814274 |
| PTPN2 | Chr18: 12814253-12814272 |
| PTPN2 | Chr18: 12814257-12814276 |
| PTPN2 | Chr18: 12814256-12814275 |
| PTPN2 | Chr18: 12840753-12840772 |
| PTPN2 | Chr18: 12830957-12830976 |
| PTPN2 | Chr18: 12802093-12802112 |
| PTPN2 | Chr18: 12817333-12817352 |
| PTPN2 | Chr18: 12794479-12794498 |
| PTPN2 | Chr18: 12814223-12814242 |
| PTPN2 | Chr18: 12802089-12802108 |
| PTPN2 | Chr18: 12794463-12794482 |
| PTPN2 | Chr18: 12794436-12794455 |
| PTPN2 | Chr18: 12794416-12794435 |
| PTPN2 | Chr18: 12817235-12817254 |
| PTPN2 | Chr18: 12836793-12836812 |
| PTPN2 | Chr18: 12801986-12802005 |
| PTPN2 | Chr18: 12817165-12817184 |
| PTPN2 | Chr18: 12817179-12817198 |
| PTPN2 | Chr18: 12794425-12794444 |
| PTPN2 | Chr18: 12802146-12802165 |

TABLE 10
Ptpn2 Murine Genome Coordinates

| Target | Coordinates |
|---|---|
| Ptpn2 | Chr18: 67680998-67681017 |
| Ptpn2 | Chr18: 67677801-67677820 |
| Ptpn2 | Chr18: 67680904-67680923 |
| Ptpn2 | Chr18: 67681553-67681572 |
| Ptpn2 | Chr18: 67688965-67688984 |
| Ptpn2 | Chr18: 67680958-67680977 |
| Ptpn2 | Chr18: 67688944-67688963 |
| Ptpn2 | Chr18: 67677855-67677874 |
| Ptpn2 | Chr18: 67677734-67677753 |
| Ptpn2 | Chr18: 67680967-67680986 |
| Ptpn2 | Chr18: 67688912-67688931 |
| Ptpn2 | Chr18: 67680881-67680900 |
| Ptpn2 | Chr18: 67681529-67681548 |

In some embodiments, the nucleic acid-based gene-regulating system comprises at least one nucleic acid molecule (e.g., an siRNA, an shRNA, an RNA aptamer, or a morpholino), wherein the at least one nucleic acid molecule is a ZC3H12A-targeting nucleic acid molecule. In some embodiments, the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6). In some embodiments, the at least one ZC3H12A-targeting nucleic acid molecule is an siRNA or an shRNA molecule. In some embodiments, the at least one ZC3H12A-targeting siRNA or an shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one ZC3H12A-targeting siRNA or an shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 11 (human genome) or Table 12 (mouse genome). In some embodiments, the at least one ZC3H12A-targeting nucleic acid molecule binds to a target RNA sequence that is 100% identical to an RNA sequence encoded by a DNA sequence defined by a set of genomic coordinates shown in Table 11 or Table 12. In some embodiments, the at least one ZC3H12A-targeting nucleic acid molecule binds to a target RNA sequence that is at least 95%, 96%, 97%, 98%, or 99% identical to an RNA sequence encoded by one of SEQ ID NOs: 331-337 or 331-797. In some embodiments, the at least one ZC3H12A-targeting nucleic acid molecule binds to a target RNA sequence that is 100% identical to an RNA sequence encoded by one of SEQ ID NOs: 331-337 or 331-797.

In some embodiments, the at least one ZC3H12A-targeting nucleic acid molecule is a ZC3H12A-targeting shRNA or siRNA molecule. In some embodiments, the at least one ZC3H12A-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one ZC3H12A-targeting shRNA or siRNA molecule binds to a target RNA sequence that is 100% identical to an RNA sequence encoded by a DNA sequence defined by a set of genomic coordinates shown in Table 11 or Table 12. In some embodiments, the ZC3H12A-targeting nucleic acid molecule is a ZC3H12A-targeting siRNA comprising a nucleic acid sequence selected from SEQ ID NOs: 328-330 or 329 and 330 (human) (See Liu et al., Scientific Reports (2016), 6, Article #24073 and Mino et al., Cell (2015) 161(5), 1058-1073, incorporated herein by reference in its entirety). In some embodiments, the at least one ZC3H12A-targeting shRNA or siRNA molecule binds to a target RNA sequence that is at least 95%, 96%, 97%, 98%, or 99% identical to an RNA sequence encoded by one of SEQ ID NOs: 331-797. In some embodiments, the at least one ZC3H12A-targeting shRNA or siRNA molecule binds to a human target RNA sequence that is at least 95%, 96%, 97%, 98%, or 99% identical to a human RNA sequence encoded by one of SEQ ID NOs: 336-789. In some embodiments, the at least one ZC3H12A-targeting shRNA or siRNA molecule binds to a target RNA sequence that is 100% identical to an RNA sequence encoded by one of SEQ ID NOs: 331-797. In some embodiments, the at least one ZC3H12A-targeting shRNA or siRNA molecule binds to a human target RNA sequence that is 100% identical to a human RNA sequence encoded by one of SEQ ID NOs: 336-789. In some embodiments, the ZC3H12A-targeting nucleic acid molecule is a ZC3H12A-targeting shRNA molecule encoded by a nucleic acid sequence selected from SEQ ID NOs: 331-337 (See Huang et al., J Biol Chem (2015) 290(34), 20782-20792, incorporated by reference herein in its entirety).

TABLE 11
ZC3H12A Human Genome Coordinates

| Target | Coordinates |
|---|---|
| ZC3H12A | Chr1: 37481708-37481727 |
| ZC3H12A | Chr1: 37475808-37475827 |
| ZC3H12A | Chr1: 37475809-37475828 |
| ZC3H12A | Chr1: 37475684-37475703 |
| ZC3H12A | Chr1: 37481823-37481842 |
| ZC3H12A | Chr1: 37480415-37480434 |
| ZC3H12A | Chr1: 37475756-37475775 |
| ZC3H12A | Chr1: 37481692-37481711 |
| ZC3H12A | Chr1: 37481648-37481667 |
| ZC3H12A | Chr1: 37480284-37480303 |
| ZC3H12A | Chr1: 37481779-37481798 |
| ZC3H12A | Chr1: 37475827-37475846 |
| ZC3H12A | Chr1: 37481747-37481766 |
| ZC3H12A | Chr1: 37482445-37482464 |
| ZC3H12A | Chr1: 37475631-37475650 |
| ZC3H12A | Chr1: 37480274-37480293 |
| ZC3H12A | Chr1: 37482967-37482986 |
| ZC3H12A | Chr1: 37482922-37482941 |
| ZC3H12A | Chr1: 37480273-37480292 |
| ZC3H12A | Chr1: 37482886-37482905 |
| ZC3H12A | Chr1: 37483185-37483204 |
| ZC3H12A | Chr1: 37475817-37475836 |
| ZC3H12A | Chr1: 37483033-37483052 |
| ZC3H12A | Chr1: 37480408-37480427 |
| ZC3H12A | Chr1: 37483026-37483045 |
| ZC3H12A | Chr1: 37483463-37483482 |
| ZC3H12A | Chr1: 37480362-37480381 |
| ZC3H12A | Chr1: 37482962-37482981 |
| ZC3H12A | Chr1: 37475775-37475794 |
| ZC3H12A | Chr1: 37475509-37475528 |
| ZC3H12A | Chr1: 37475722-37475741 |
| ZC3H12A | Chr1: 37475818-37475837 |
| ZC3H12A | Chr1: 37482966-37482985 |
| ZC3H12A | Chr1: 37480388-37480407 |
| ZC3H12A | Chr1: 37483142-37483161 |
| ZC3H12A | Chr1: 37482448-37482467 |
| ZC3H12A | Chr1: 37483049-37483068 |
| ZC3H12A | Chr1: 37482905-37482924 |
| ZC3H12A | Chr1: 37482733-37482752 |
| ZC3H12A | Chr1: 37480423-37480442 |
| ZC3H12A | Chr1: 37482456-37482475 |
| ZC3H12A | Chr1: 37483551-37483570 |
| ZC3H12A | Chr1: 37481767-37481786 |
| ZC3H12A | Chr1: 37475715-37475734 |
| ZC3H12A | Chr1: 37483377-37483396 |
| ZC3H12A | Chr1: 37475593-37475612 |
| ZC3H12A | Chr1: 37475875-37475894 |
| ZC3H12A | Chr1: 37475534-37475553 |
| ZC3H12A | Chr1: 37482764-37482783 |
| ZC3H12A | Chr1: 37475869-37475888 |
| ZC3H12A | Chr1: 37483437-37483456 |
| ZC3H12A | Chr1: 37475598-37475617 |
| ZC3H12A | Chr1: 37482438-37482457 |
| ZC3H12A | Chr1: 37483257-37483276 |
| ZC3H12A | Chr1: 37483263-37483282 |
| ZC3H12A | Chr1: 37482545-37482564 |
| ZC3H12A | Chr1: 37483015-37483034 |
| ZC3H12A | Chr1: 37481595-37481614 |
| ZC3H12A | Chr1: 37482923-37482942 |
| ZC3H12A | Chr1: 37483143-37483162 |
| ZC3H12A | Chr1: 37482348-37482367 |
| ZC3H12A | Chr1: 37483018-37483037 |
| ZC3H12A | Chr1: 37482612-37482631 |
| ZC3H12A | Chr1: 37475613-37475632 |
| ZC3H12A | Chr1: 37475563-37475582 |
| ZC3H12A | Chr1: 37475535-37475554 |
| ZC3H12A | Chr1: 37482843-37482862 |
| ZC3H12A | Chr1: 37480424-37480443 |
| ZC3H12A | Chr1: 37482606-37482625 |
| ZC3H12A | Chr1: 37483098-37483117 |
| ZC3H12A | Chr1: 37483508-37483527 |
| ZC3H12A | Chr1: 37483559-37483578 |
| ZC3H12A | Chr1: 37483256-37483275 |
| ZC3H12A | Chr1: 37475936-37475955 |
| ZC3H12A | Chr1: 37475607-37475626 |
| ZC3H12A | Chr1: 37475809-37475828 |
| ZC3H12A | Chr1: 37483186-37483205 |
| ZC3H12A | Chr1: 37481747-37481766 |
| ZC3H12A | Chr1: 37482734-37482753 |
| ZC3H12A | Chr1: 37483278-37483297 |
| ZC3H12A | Chr1: 37482332-37482351 |
| ZC3H12A | Chr1: 37483109-37483128 |
| ZC3H12A | Chr1: 37475633-37475652 |
| ZC3H12A | Chr1: 37482591-37482610 |
| ZC3H12A | Chr1: 37483271-37483290 |
| ZC3H12A | Chr1: 37483603-37483622 |
| ZC3H12A | Chr1: 37482504-37482523 |
| ZC3H12A | Chr1: 37483252-37483271 |
| ZC3H12A | Chr1: 37483119-37483138 |
| ZC3H12A | Chr1: 37482343-37482362 |
| ZC3H12A | Chr1: 37483144-37483163 |
| ZC3H12A | Chr1: 37483213-37483232 |
| ZC3H12A | Chr1: 37482981-37483000 |
| ZC3H12A | Chr1: 37482789-37482808 |
| ZC3H12A | Chr1: 37483159-37483178 |
| ZC3H12A | Chr1: 37482349-37482368 |
| ZC3H12A | Chr1: 37483602-37483621 |
| ZC3H12A | Chr1: 37481596-37481615 |
| ZC3H12A | Chr1: 37482537-37482556 |
| ZC3H12A | Chr1: 37482370-37482389 |
| ZC3H12A | Chr1: 37475546-37475565 |
| ZC3H12A | Chr1: 37482598-37482617 |
| ZC3H12A | Chr1: 37483146-37483165 |
| ZC3H12A | Chr1: 37475812-37475831 |
| ZC3H12A | Chr1: 37483400-37483419 |
| ZC3H12A | Chr1: 37475703-37475722 |
| ZC3H12A | Chr1: 37483418-37483437 |
| ZC3H12A | Chr1: 37480284-37480303 |
| ZC3H12A | Chr1: 37482800-37482819 |
| ZC3H12A | Chr1: 37475721-37475740 |
| ZC3H12A | Chr1: 37482715-37482734 |
| ZC3H12A | Chr1: 37480281-37480300 |
| ZC3H12A | Chr1: 37482491-37482510 |
| ZC3H12A | Chr1: 37483497-37483516 |
| ZC3H12A | Chr1: 37475899-37475918 |
| ZC3H12A | Chr1: 37475889-37475908 |
| ZC3H12A | Chr1: 37482375-37482394 |
| ZC3H12A | Chr1: 37475741-37475760 |
| ZC3H12A | Chr1: 37482900-37482919 |
| ZC3H12A | Chr1: 37482442-37482461 |
| ZC3H12A | Chr1: 37481644-37481663 |
| ZC3H12A | Chr1: 37482464-37482483 |
| ZC3H12A | Chr1: 37482994-37483013 |
| ZC3H12A | Chr1: 37483437-37483456 |
| ZC3H12A | Chr1: 37482736-37482755 |
| ZC3H12A | Chr1: 37482538-37482557 |
| ZC3H12A | Chr1: 37483515-37483534 |
| ZC3H12A | Chr1: 37475874-37475893 |
| ZC3H12A | Chr1: 37483145-37483164 |
| ZC3H12A | Chr1: 37482587-37482606 |
| ZC3H12A | Chr1: 37475482-37475501 |
| ZC3H12A | Chr1: 37475844-37475863 |
| ZC3H12A | Chr1: 37480415-37480434 |
| ZC3H12A | Chr1: 37481709-37481728 |
| ZC3H12A | Chr1: 37483366-37483385 |
| ZC3H12A | Chr1: 37475627-37475646 |
| ZC3H12A | Chr1: 37482447-37482466 |
| ZC3H12A | Chr1: 37481758-37481777 |
| ZC3H12A | Chr1: 37483560-37483579 |
| ZC3H12A | Chr1: 37475869-37475888 |
| ZC3H12A | Chr1: 37481655-37481674 |
| ZC3H12A | Chr1: 37481645-37481664 |
| ZC3H12A | Chr1: 37483016-37483035 |
| ZC3H12A | Chr1: 37475838-37475857 |
| ZC3H12A | Chr1: 37482850-37482869 |
| ZC3H12A | Chr1: 37475510-37475529 |
| ZC3H12A | Chr1: 37483510-37483529 |
| ZC3H12A | Chr1: 37483064-37483083 |
| ZC3H12A | Chr1: 37483149-37483168 |
| ZC3H12A | Chr1: 37483449-37483468 |
| ZC3H12A | Chr1: 37483264-37483283 |
| ZC3H12A | Chr1: 37475508-37475527 |
| ZC3H12A | Chr1: 37480415-37480434 |
| ZC3H12A | Chr1: 37482918-37482937 |
| ZC3H12A | Chr1: 37482474-37482493 |
| ZC3H12A | Chr1: 37483232-37483251 |
| ZC3H12A | Chr1: 37475732-37475751 |
| ZC3H12A | Chr1: 37481602-37481621 |
| ZC3H12A | Chr1: 37480289-37480308 |
| ZC3H12A | Chr1: 37483165-37483184 |
| ZC3H12A | Chr1: 37483248-37483267 |
| ZC3H12A | Chr1: 37483078-37483097 |
| ZC3H12A | Chr1: 37483017-37483036 |
| ZC3H12A | Chr1: 37483174-37483193 |
| ZC3H12A | Chr1: 37482857-37482876 |
| ZC3H12A | Chr1: 37475578-37475597 |
| ZC3H12A | Chr1: 37480329-37480348 |
| ZC3H12A | Chr1: 37480288-37480307 |
| ZC3H12A | Chr1: 37481600-37481619 |
| ZC3H12A | Chr1: 37483212-37483231 |
| ZC3H12A | Chr1: 37483337-37483356 |
| ZC3H12A | Chr1: 37475542-37475561 |
| ZC3H12A | Chr1: 37483197-37483216 |
| ZC3H12A | Chr1: 37482730-37482749 |
| ZC3H12A | Chr1: 37475599-37475618 |
| ZC3H12A | Chr1: 37483262-37483281 |
| ZC3H12A | Chr1: 37482790-37482809 |
| ZC3H12A | Chr1: 37482719-37482738 |
| ZC3H12A | Chr1: 37482860-37482879 |
| ZC3H12A | Chr1: 37483443-37483462 |
| ZC3H12A | Chr1: 37483558-37483577 |
| ZC3H12A | Chr1: 37481599-37481618 |
| ZC3H12A | Chr1: 37475845-37475864 |
| ZC3H12A | Chr1: 37475730-37475749 |
| ZC3H12A | Chr1: 37482524-37482543 |
| ZC3H12A | Chr1: 37482849-37482868 |
| ZC3H12A | Chr1: 37475529-37475548 |
| ZC3H12A | Chr1: 37475664-37475683 |
| ZC3H12A | Chr1: 37482972-37482991 |
| ZC3H12A | Chr1: 37483321-37483340 |
| ZC3H12A | Chr1: 37482984-37483003 |
| ZC3H12A | Chr1: 37475807-37475826 |
| ZC3H12A | Chr1: 37483213-37483232 |
| ZC3H12A | Chr1: 37482427-37482446 |
| ZC3H12A | Chr1: 37483104-37483123 |
| ZC3H12A | Chr1: 37482879-37482898 |
| ZC3H12A | Chr1: 37483409-37483428 |
| ZC3H12A | Chr1: 37482752-37482771 |
| ZC3H12A | Chr1: 37480391-37480410 |
| ZC3H12A | Chr1: 37475694-37475713 |
| ZC3H12A | Chr1: 37482458-37482477 |
| ZC3H12A | Chr1: 37475774-37475793 |
| ZC3H12A | Chr1: 37475574-37475593 |
| ZC3H12A | Chr1: 37475803-37475822 |
| ZC3H12A | Chr1: 37481605-37481624 |
| ZC3H12A | Chr1: 37482437-37482456 |
| ZC3H12A | Chr1: 37482825-37482844 |
| ZC3H12A | Chr1: 37483595-37483614 |
| ZC3H12A | Chr1: 37483510-37483529 |
| ZC3H12A | Chr1: 37483283-37483302 |
| ZC3H12A | Chr1: 37482446-37482465 |
| ZC3H12A | Chr1: 37475700-37475719 |
| ZC3H12A | Chr1: 37475721-37475740 |
| ZC3H12A | Chr1: 37475628-37475647 |
| ZC3H12A | Chr1: 37482848-37482867 |
| ZC3H12A | Chr1: 37483134-37483153 |
| ZC3H12A | Chr1: 37475543-37475562 |
| ZC3H12A | Chr1: 37482799-37482818 |
| ZC3H12A | Chr1: 37483296-37483315 |
| ZC3H12A | Chr1: 37483332-37483351 |
| ZC3H12A | Chr1: 37483600-37483619 |
| ZC3H12A | Chr1: 37482410-37482429 |
| ZC3H12A | Chr1: 37481718-37481737 |
| ZC3H12A | Chr1: 37483395-37483414 |
| ZC3H12A | Chr1: 37482428-37482447 |
| ZC3H12A | Chr1: 37475562-37475581 |
| ZC3H12A | Chr1: 37483500-37483519 |
| ZC3H12A | Chr1: 37475827-37475846 |
| ZC3H12A | Chr1: 37483586-37483605 |
| ZC3H12A | Chr1: 37483089-37483108 |
| ZC3H12A | Chr1: 37483419-37483438 |
| ZC3H12A | Chr1: 37480285-37480304 |
| ZC3H12A | Chr1: 37483256-37483275 |
| ZC3H12A | Chr1: 37483420-37483439 |
| ZC3H12A | Chr1: 37475691-37475710 |
| ZC3H12A | Chr1: 37483419-37483438 |
| ZC3H12A | Chr1: 37475918-37475937 |
| ZC3H12A | Chr1: 37475589-37475608 |
| ZC3H12A | Chr1: 37482362-37482381 |
| ZC3H12A | Chr1: 37482566-37482585 |
| ZC3H12A | Chr1: 37482963-37482982 |
| ZC3H12A | Chr1: 37483420-37483439 |
| ZC3H12A | Chr1: 37483139-37483158 |
| ZC3H12A | Chr1: 37483619-37483638 |
| ZC3H12A | Chr1: 37481764-37481783 |
| ZC3H12A | Chr1: 37475650-37475669 |
| ZC3H12A | Chr1: 37483405-37483424 |
| ZC3H12A | Chr1: 37483037-37483056 |
| ZC3H12A | Chr1: 37483211-37483230 |
| ZC3H12A | Chr1: 37475537-37475556 |
| ZC3H12A | Chr1: 37475756-37475775 |
| ZC3H12A | Chr1: 37482403-37482422 |
| ZC3H12A | Chr1: 37482455-37482474 |
| ZC3H12A | Chr1: 37480311-37480330 |
| ZC3H12A | Chr1: 37482586-37482605 |
| ZC3H12A | Chr1: 37483099-37483118 |
| ZC3H12A | Chr1: 37483342-37483361 |
| ZC3H12A | Chr1: 37481823-37481842 |
| ZC3H12A | Chr1: 37482777-37482796 |
| ZC3H12A | Chr1: 37482412-37482431 |
| ZC3H12A | Chr1: 37483604-37483623 |
| ZC3H12A | Chr1: 37483438-37483457 |
| ZC3H12A | Chr1: 37482445-37482464 |
| ZC3H12A | Chr1: 37483331-37483350 |
| ZC3H12A | Chr1: 37483111-37483130 |
| ZC3H12A | Chr1: 37482847-37482866 |
| ZC3H12A | Chr1: 37483249-37483268 |
| ZC3H12A | Chr1: 37481754-37481773 |
| ZC3H12A | Chr1: 37475684-37475703 |
| ZC3H12A | Chr1: 37482519-37482538 |
| ZC3H12A | Chr1: 37482475-37482494 |
| ZC3H12A | Chr1: 37482613-37482632 |
| ZC3H12A | Chr1: 37482939-37482958 |
| ZC3H12A | Chr1: 37475541-37475560 |
| ZC3H12A | Chr1: 37481763-37481782 |
| ZC3H12A | Chr1: 37483231-37483250 |
| ZC3H12A | Chr1: 37482953-37482972 |
| ZC3H12A | Chr1: 37482407-37482426 |
| ZC3H12A | Chr1: 37475808-37475827 |
| ZC3H12A | Chr1: 37481620-37481639 |
| ZC3H12A | Chr1: 37475592-37475611 |
| ZC3H12A | Chr1: 37483156-37483175 |
| ZC3H12A | Chr1: 37480329-37480348 |
| ZC3H12A | Chr1: 37475573-37475592 |
| ZC3H12A | Chr1: 37483198-37483217 |
| ZC3H12A | Chr1: 37483557-37483576 |
| ZC3H12A | Chr1: 37482892-37482911 |
| ZC3H12A | Chr1: 37483334-37483353 |
| ZC3H12A | Chr1: 37481708-37481727 |
| ZC3H12A | Chr1: 37483063-37483082 |
| ZC3H12A | Chr1: 37482998-37483017 |
| ZC3H12A | Chr1: 37482942-37482961 |
| ZC3H12A | Chr1: 37475508-37475527 |
| ZC3H12A | Chr1: 37482371-37482390 |
| ZC3H12A | Chr1: 37483119-37483138 |
| ZC3H12A | Chr1: 37482798-37482817 |
| ZC3H12A | Chr1: 37475859-37475878 |
| ZC3H12A | Chr1: 37483401-37483420 |
| ZC3H12A | Chr1: 37482851-37482870 |
| ZC3H12A | Chr1: 37475524-37475543 |
| ZC3H12A | Chr1: 37475601-37475620 |
| ZC3H12A | Chr1: 37475815-37475834 |
| ZC3H12A | Chr1: 37482801-37482820 |
| ZC3H12A | Chr1: 37475544-37475563 |
| ZC3H12A | Chr1: 37483010-37483029 |
| ZC3H12A | Chr1: 37483077-37483096 |
| ZC3H12A | Chr1: 37482404-37482423 |
| ZC3H12A | Chr1: 37475692-37475711 |
| ZC3H12A | Chr1: 37483596-37483615 |
| ZC3H12A | Chr1: 37483372-37483391 |
| ZC3H12A | Chr1: 37481596-37481615 |
| ZC3H12A | Chr1: 37480370-37480389 |
| ZC3H12A | Chr1: 37480377-37480396 |
| ZC3H12A | Chr1: 37483381-37483400 |
| ZC3H12A | Chr1: 37482899-37482918 |
| ZC3H12A | Chr1: 37480373-37480392 |
| ZC3H12A | Chr1: 37481847-37481866 |
| ZC3H12A | Chr1: 37483330-37483349 |
| ZC3H12A | Chr1: 37483065-37483084 |
| ZC3H12A | Chr1: 37482499-37482518 |
| ZC3H12A | Chr1: 37483105-37483124 |
| ZC3H12A | Chr1: 37475631-37475650 |
| ZC3H12A | Chr1: 37483530-37483549 |
| ZC3H12A | Chr1: 37483407-37483426 |
| ZC3H12A | Chr1: 37483308-37483327 |
| ZC3H12A | Chr1: 37482853-37482872 |
| ZC3H12A | Chr1: 37482934-37482953 |
| ZC3H12A | Chr1: 37475591-37475610 |
| ZC3H12A | Chr1: 37475826-37475845 |
| ZC3H12A | Chr1: 37475865-37475884 |
| ZC3H12A | Chr1: 37481784-37481803 |
| ZC3H12A | Chr1: 37480322-37480341 |
| ZC3H12A | Chr1: 37475664-37475683 |
| ZC3H12A | Chr1: 37475757-37475776 |
| ZC3H12A | Chr1: 37483385-37483404 |
| ZC3H12A | Chr1: 37482933-37482952 |
| ZC3H12A | Chr1: 37475866-37475885 |
| ZC3H12A | Chr1: 37475843-37475862 |
| ZC3H12A | Chr1: 37475797-37475816 |
| ZC3H12A | Chr1: 37475642-37475661 |
| ZC3H12A | Chr1: 37483270-37483289 |
| ZC3H12A | Chr1: 37483024-37483043 |
| ZC3H12A | Chr1: 37483201-37483220 |
| ZC3H12A | Chr1: 37482447-37482466 |
| ZC3H12A | Chr1: 37483253-37483272 |
| ZC3H12A | Chr1: 37483429-37483448 |
| ZC3H12A | Chr1: 37483195-37483214 |
| ZC3H12A | Chr1: 37481648-37481667 |
| ZC3H12A | Chr1: 37483424-37483443 |
| ZC3H12A | Chr1: 37475580-37475599 |
| ZC3H12A | Chr1: 37482980-37482999 |
| ZC3H12A | Chr1: 37480408-37480427 |
| ZC3H12A | Chr1: 37483405-37483424 |
| ZC3H12A | Chr1: 37475740-37475759 |
| ZC3H12A | Chr1: 37480387-37480406 |
| ZC3H12A | Chr1: 37483507-37483526 |
| ZC3H12A | Chr1: 37483110-37483129 |
| ZC3H12A | Chr1: 37483325-37483344 |
| ZC3H12A | Chr1: 37481692-37481711 |
| ZC3H12A | Chr1: 37475826-37475845 |
| ZC3H12A | Chr1: 37483098-37483117 |
| ZC3H12A | Chr1: 37481758-37481777 |
| ZC3H12A | Chr1: 37480320-37480339 |
| ZC3H12A | Chr1: 37483380-37483399 |
| ZC3H12A | Chr1: 37483011-37483030 |
| ZC3H12A | Chr1: 37483509-37483528 |
| ZC3H12A | Chr1: 37483509-37483528 |
| ZC3H12A | Chr1: 37482768-37482787 |
| ZC3H12A | Chr1: 37475804-37475823 |
| ZC3H12A | Chr1: 37475808-37475827 |
| ZC3H12A | Chr1: 37475859-37475878 |
| ZC3H12A | Chr1: 37482973-37482992 |
| ZC3H12A | Chr1: 37475634-37475653 |
| ZC3H12A | Chr1: 37475854-37475873 |
| ZC3H12A | Chr1: 37480334-37480353 |
| ZC3H12A | Chr1: 37480414-37480433 |
| ZC3H12A | Chr1: 37480316-37480335 |
| ZC3H12A | Chr1: 37482971-37482990 |
| ZC3H12A | Chr1: 37482781-37482800 |
| ZC3H12A | Chr1: 37483173-37483192 |
| ZC3H12A | Chr1: 37482391-37482410 |
| ZC3H12A | Chr1: 37482392-37482411 |
| ZC3H12A | Chr1: 37482936-37482955 |
| ZC3H12A | Chr1: 37483408-37483427 |
| ZC3H12A | Chr1: 37481779-37481798 |
| ZC3H12A | Chr1: 37483206-37483225 |
| ZC3H12A | Chr1: 37482561-37482580 |
| ZC3H12A | Chr1: 37481745-37481764 |
| ZC3H12A | Chr1: 37475802-37475821 |
| ZC3H12A | Chr1: 37483494-37483513 |
| ZC3H12A | Chr1: 37483371-37483390 |
| ZC3H12A | Chr1: 37482552-37482571 |
| ZC3H12A | Chr1: 37475491-37475510 |
| ZC3H12A | Chr1: 37482479-37482498 |
| ZC3H12A | Chr1: 37483140-37483159 |
| ZC3H12A | Chr1: 37483313-37483332 |
| ZC3H12A | Chr1: 37483458-37483477 |
| ZC3H12A | Chr1: 37483320-37483339 |
| ZC3H12A | Chr1: 37483204-37483223 |
| ZC3H12A | Chr1: 37475792-37475811 |
| ZC3H12A | Chr1: 37483475-37483494 |
| ZC3H12A | Chr1: 37475577-37475596 |
| ZC3H12A | Chr1: 37475787-37475806 |
| ZC3H12A | Chr1: 37483574-37483593 |
| ZC3H12A | Chr1: 37480284-37480303 |
| ZC3H12A | Chr1: 37482369-37482388 |
| ZC3H12A | Chr1: 37483384-37483403 |
| ZC3H12A | Chr1: 37483425-37483444 |
| ZC3H12A | Chr1: 37482582-37482601 |
| ZC3H12A | Chr1: 37483153-37483172 |
| ZC3H12A | Chr1: 37482935-37482954 |
| ZC3H12A | Chr1: 37483378-37483397 |
| ZC3H12A | Chr1: 37482952-37482971 |
| ZC3H12A | Chr1: 37483399-37483418 |
| ZC3H12A | Chr1: 37483309-37483328 |
| ZC3H12A | Chr1: 37483200-37483219 |
| ZC3H12A | Chr1: 37481641-37481660 |
| ZC3H12A | Chr1: 37481656-37481675 |
| ZC3H12A | Chr1: 37483036-37483055 |
| ZC3H12A | Chr1: 37483474-37483493 |
| ZC3H12A | Chr1: 37483004-37483023 |
| ZC3H12A | Chr1: 37481846-37481865 |
| ZC3H12A | Chr1: 37483205-37483224 |
| ZC3H12A | Chr1: 37483406-37483425 |
| ZC3H12A | Chr1: 37480336-37480355 |
| ZC3H12A | Chr1: 37481716-37481735 |
| ZC3H12A | Chr1: 37480335-37480354 |
| ZC3H12A | Chr1: 37481659-37481678 |
| ZC3H12A | Chr1: 37475809-37475828 |
| ZC3H12A | Chr1: 37482565-37482584 |
| ZC3H12A | Chr1: 37482491-37482510 |
| ZC3H12A | Chr1: 37483379-37483398 |
| ZC3H12A | Chr1: 37481654-37481673 |
| ZC3H12A | Chr1: 37482567-37482586 |
| ZC3H12A | Chr1: 37481614-37481633 |
| ZC3H12A | Chr1: 37482562-37482581 |
| ZC3H12A | Chr1: 37475868-37475887 |
| ZC3H12A | Chr1: 37482557-37482576 |
| ZC3H12A | Chr1: 37483511-37483530 |
| ZC3H12A | Chr1: 37475615-37475634 |
| ZC3H12A | Chr1: 37483333-37483352 |
| ZC3H12A | Chr1: 37482840-37482859 |
| ZC3H12A | Chr1: 37483545-37483564 |
| ZC3H12A | Chr1: 37482830-37482849 |
| ZC3H12A | Chr1: 37482444-37482463 |
| ZC3H12A | Chr1: 37482571-37482590 |
| ZC3H12A | Chr1: 37482553-37482572 |
| ZC3H12A | Chr1: 37483543-37483562 |
| ZC3H12A | Chr1: 37483542-37483561 |
| ZC3H12A | Chr1: 37482575-37482594 |
| ZC3H12A | Chr1: 37475855-37475874 |
| ZC3H12A | Chr1: 37482572-37482591 |

TABLE 12
Zc3h12a Murine Genome Coordinates

| Target | Coordinates |
|---|---|
| Zc3h12a | Chr1: 125122335-125122354 |
| Zc3h12a | Chr1: 125121083-125121102 |
| Zc3h12a | Chr1: 125120961-125120980 |
| Zc3h12a | Chr1: 125122390-125122409 |
| Zc3h12a | Chr1: 125120373-125120392 |
| Zc3h12a | Chr1: 125122250-125122269 |
| Zc3h12a | Chr1: 125122375-125122394 |
| Zc3h12a | Chr1: 125120975-125120994 |

TABLE 13
Exemplary murine Zc3h12a siRNA sequence

| Target | Sequence | SEQ ID |
|---|---|---|
| Zc3h12a_siRNA_1 | CCUGGACAACUUCCUUCGUAAGAAA | 328 |

TABLE 14
Exemplary human ZC3H12A siRNA sequences

| Target | Sequence | SEQ ID |
|---|---|---|
| ZC3H12A_siRNA_2 | GUGUCCCUAUGGAAGGAAA | 329 |
| ZC3H12A_siRNA_3 | CAACUUCCUUCGUAAGAAA | 330 |

TABLE 15
Murine Zc3h12a shRNA and siRNA target
sequences

| | | |
|---|---|---|
| Zc3h12a_shRNA_1 | AGCGAGGCCACACAGATATTA | 331 |
| Zc3h12a_shRNA_2 | GCTATGATGACCGCTTCATTG | 332 |
| Zc3h12a_shRNA_3 | TGGTCTGAGCCGTACCCATTA | 333 |
| Zc3h12a_shRNA_4 | CTGTGTACAGAGGCGAGATTT | 334 |
| Zc3h12a_siRNA_1 | CCTGGACAACTTCCTTCGTAAGAAA | 335 |

TABLE 16
Human ZC3H12A shRNA and siRNA target
sequences

| Target | Sequence | SEQ ID |
|---|---|---|
| ZC3H12A_siRNA_2 | GTGTCCCTATGGAAGGAAA | 336 |
| ZC3H12A_siRNA_3 | CAACTTCCTTCGTAAGAAA | 337 |

In some embodiments, the nucleic acid-based gene-regulating system comprises at least one nucleic acid molecule (e.g., an siRNA, an shRNA, an RNA aptamer, or a morpholino), wherein the at least one nucleic acid molecule is a CBLB-targeting nucleic acid molecule. In some embodiments, the at least one CBLB-targeting nucleic acid molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8). In some embodiments, the at least one CBLB-targeting nucleic acid molecule binds to a target RNA sequence that is 100% identical to an RNA sequence encoded by the CBLB gene (SEQ ID NO: 7) or the Cblb gene (SEQ ID NO: 8). In some embodiments, the at least one CBLB-targeting nucleic acid molecule is an siRNA or an shRNA molecule. In some embodiments, the at least one CBLB-targeting siRNA or an shRNA molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8). In some embodiments, the at least one CBLB-targeting siRNA or an shRNA molecule binds to a target RNA sequence that is 100% identical to an RNA sequence encoded by the CBLB gene (SEQ ID NO: 7) or the Cblb gene (SEQ ID NO: 8).

In some embodiments, the at least one CBLB-targeting nucleic acid molecule binds to a target RNA sequence that is at least 95%, 96%, 97%, 98%, or 99% identical to a RNA sequence encoded by a DNA sequence defined by a set of genomic coordinates shown in Table 17 (human genome) or Table 18 (mouse genome). In some embodiments, the at least one CBLB-targeting nucleic acid molecule binds to a target RNA sequence that is 100% identical to a RNA sequence encoded by a DNA sequence defined by a set of genomic coordinates shown in Table 17 or Table 18. In some embodiments, the at least one CBLB-targeting nucleic acid molecule binds to a target RNA sequence that is at least 95%, 96%, 97%, 98%, or 99% identical to an RNA sequence encoded by one of SEQ ID NOs: 798-823. In some embodiments, the at least one CBLB-targeting nucleic acid molecule binds to a human target RNA sequence that is at least 95%, 96%, 97%, 98%, or 99% identical to a human RNA sequence encoded by one of SEQ ID NOs: 798-808. In some embodiments, the at least one CBLB-targeting nucleic acid molecule binds to a target RNA sequence that is 100% identical to an RNA sequence encoded by one of SEQ ID NOs: 798-823. In some embodiments, the at least one CBLB-targeting nucleic acid molecule binds to a human target RNA sequence that is 100% identical to a human RNA sequence encoded by one of SEQ ID NOs: 798-808.

In some embodiments, the at least one CBLB-targeting nucleic acid molecule is a CBLB-targeting shRNA or siRNA molecule. In some embodiments, the at least one CBLB-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 17 or Table 18. In some embodiments, the at least one CBLB-targeting shRNA or siRNA molecule binds to a target RNA sequence that is 100% identical to a RNA sequence encoded by a DNA sequence defined by a set of genomic coordinates shown in Table 17 or Table 18. In some embodiments, the at least one CBLB-targeting shRNA or siRNA molecule binds to a target RNA sequence that is at least 95%, 96%, 97%, 98%, or 99% identical to an RNA sequence encoded by one of SEQ ID NOs: 798-823. In some embodiments, the at least one CBLB-targeting shRNA or siRNA molecule binds to a human target RNA sequence that is at least 95%, 96%, 97%, 98%, or 99% identical to a human RNA sequence encoded by one of SEQ ID NOs: 798-808. In some embodiments, the at least one CBLB-targeting shRNA or siRNA molecule binds to a target RNA sequence that is 100% identical to an RNA sequence encoded by one of SEQ ID NOs: 798-823. In some embodiments, the at least one CBLB-targeting shRNA or siRNA molecule binds to a human target RNA sequence that is 100% identical to a human RNA sequence encoded by one of SEQ ID NOs: 798-808.

TABLE 17
CBLB Human Genome Coordinates

| Target | Coordinates |
|---|---|
| CBLB | chr3: 105853475-105853494 |
| CBLB | chr3: 105853600-105853619 |
| CBLB | chr3: 105720111-105720130 |
| CBLB | chr3: 105867412-105867431 |
| CBLB | chr3: 105867529-105867548 |
| CBLB | chr3: 105720160-105720179 |
| CBLB | chr3: 105853421-105853440 |
| CBLB | chr3: 105751453-105751472 |
| CBLB | chr3: 105693541-105693560 |
| CBLB | chr3: 105867449-105867468 |
| CBLB | chr3: 105853514-105853533 |

TABLE 18
Cblb Mouse Genome Coordinates

| Target | Coordinates |
|---|---|
| Cblb | chr16: 52152499-52152518 |
| Cblb | chr16: 52139574-52139593 |
| Cblb | chr16: 52139603-52139622 |
| Cblb | chr16: 52112122-52112141 |
| Cblb | chr16: 52112134-52112153 |
| Cblb | chr16: 52152535-52152554 |
| Cblb | chr16: 52142891-52142910 |
| Cblb | chr16: 52135797-52135816 |
| Cblb | chr16: 52131105-52131124 |
| Cblb | chr16: 52112169-52112188 |
| Cblb | chr16: 52204542-52204561 |
| Cblb | chr16: 52131058-52131077 |
| Cblb | chr16: 52135876-52135895 |
| Cblb | chr16: 52135763-52135782 |
| Cblb | chr16: 52139509-52139528 |

In some embodiments, the nucleic acid-based gene-regulating system comprises at least one nucleic acid molecule (e.g., an siRNA, an shRNA, an RNA aptamer, or a morpholino), wherein the at least one nucleic acid molecule is a RC3H1-targeting nucleic acid molecule. In some embodiments, the at least one RC3H1-targeting nucleic acid molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10). In some embodiments, the at least one RC3H1-targeting nucleic acid molecule binds to a target RNA sequence that is 100% identical to an RNA sequence encoded by the RC3H1 gene (SEQ ID NO: 9) or the Rc3h1 gene (SEQ ID NO: 10). In some embodiments, the at least one RC3H1-targeting nucleic acid molecule is an siRNA or an shRNA molecule. In some embodiments, the at least one RC3H1-targeting siRNA or an shRNA molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10). In some embodiments, the at least one RC3H1-targeting siRNA or an shRNA molecule binds to a target RNA sequence that is 100% identical to an RNA sequence encoded by the RC3H1 gene (SEQ ID NO: 9) or the Rc3h1 gene (SEQ ID NO: 10).

In some embodiments, the at least one RC3H1-targeting nucleic acid molecule binds to a target RNA sequence that is at least 95%, 96%, 97%, 98%, or 99% identical to a RNA sequence encoded by a DNA sequence defined by a set of genomic coordinates shown in Table 19 (human genome) or Table 20 (mouse genome). In some embodiments, the at least one RC3H1-targeting nucleic acid molecule binds to a target RNA sequence that is 100% identical to a RNA sequence encoded by a DNA sequence defined by a set of genomic coordinates shown in Table 19 or Table 20. In some embodiments, the at least one RC3H1-targeting nucleic acid molecule binds to a target RNA sequence that is at least 95%, 96%, 97%, 98%, or 99% identical to an RNA sequence encoded by one of SEQ ID NOs: 824-844. In some embodiments, the at least one RC3H1-targeting nucleic acid molecule binds to a human target RNA sequence that is at least 95%, 96%, 97%, 98%, or 99% identical to a human RNA sequence encoded by one of SEQ ID NOs: 824-836. In some embodiments, the at least one RC3H1-targeting nucleic acid molecule binds to a target RNA sequence that is 100% identical to an RNA sequence encoded by one of SEQ ID NOs: 824-844. In some embodiments, the at least one RC3H1-targeting nucleic acid molecule binds to a human target RNA sequence that is 100% identical to a human RNA sequence encoded by one of SEQ ID NOs: 824-836.

In some embodiments, the at least one RC3H1-targeting nucleic acid molecule is a RC3H1-targeting shRNA or siRNA molecule. In some embodiments, the at least one RC3H1-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 19 or Table 20. In some embodiments, the at least one RC3H1-targeting shRNA or siRNA molecule binds to a target RNA sequence that is 100% identical to a RNA sequence encoded by a DNA sequence defined by a set of genomic coordinates shown in Table 19 or Table 20. In some embodiments, the at least one RC3H1-targeting shRNA or siRNA molecule binds to a target RNA sequence that is at least 95%, 96%, 97%, 98%, or 99% identical to an RNA sequence encoded by one of SEQ ID NOs: 824-844. In some embodiments, the at least one RC3H1-targeting shRNA or siRNA molecule binds to a human target RNA sequence that is at least 95%, 96%, 97%, 98%, or 99% identical to a human RNA sequence encoded by one of SEQ ID NOs: 824-836. In some embodiments, the at least one RC3H1-targeting shRNA or siRNA molecule binds to a target RNA sequence that is 100% identical to an RNA sequence encoded by one of SEQ ID NOs: 824-844. In some embodiments, the at least one RC3H1-targeting shRNA or siRNA molecule binds to a human target RNA sequence that is 100% identical to a human RNA sequence encoded by one of SEQ ID NOs: 824-836.

TABLE 19
RC3H1 Human Genome Coordinates

| Target | Coordinates |
|---|---|
| RC3H1 | chr1: 173946812-173946831 |
| RC3H1 | chr1: 173992926-173992945 |
| RC3H1 | chr1: 173980872-173980891 |
| RC3H1 | chr1: 173982779-173982798 |
| RC3H1 | chr1: 173980941-173980960 |
| RC3H1 | chr1: 173992844-173992863 |
| RC3H1 | chr1: 173992895-173992914 |
| RC3H1 | chr1: 173992882-173992901 |
| RC3H1 | chr1: 173961717-173961736 |
| RC3H1 | chr1: 173984495-173984514 |
| RC3H1 | chr1: 173980811-173980830 |
| RC3H1 | chr1: 173964926-173964945 |
| RC3H1 | chr1: 173982894-173982913 |

TABLE 20
Rc3h1 Mouse Genome Coordinates

| Target | Coordinates |
|---|---|
| Rc3h1 | chr1: 160930251-160930270 |
| Rc3h1 | chr1: 160930280-160930299 |
| Rc3h1 | chr1: 160930154-160930173 |
| Rc3h1 | chr1: 160942614-160942633 |
| Rc3h1 | chr1: 160930266-160930285 |
| Rc3h1 | chr1: 160930185-160930204 |
| Rc3h1 | chr1: 160938126-160938145 |
| Rc3h1 | chr1: 160930198-160930217 |

In some embodiments, the nucleic acid-based gene-regulating system comprises at least one nucleic acid molecule (e.g., an siRNA, an shRNA, an RNA aptamer, or a morpholino), wherein the at least one nucleic acid molecule is a NFKBIA-targeting nucleic acid molecule. In some embodiments, the at least one NFKBIA-targeting nucleic acid molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one NFKBIA-targeting nucleic acid molecule binds to a target RNA sequence that is 100% identical to an RNA sequence encoded by the NFKBIA gene (SEQ ID NO: 11) or the Nfkbia gene (SEQ ID NO: 12). In some embodiments, the at least one NFKBIA-targeting nucleic acid molecule is an siRNA or an shRNA molecule. In some embodiments, the at least one NFKBIA-targeting siRNA or an shRNA molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one NFKBIA-targeting siRNA or an shRNA molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one NFKBIA-targeting nucleic acid molecule binds to a target RNA sequence that is at least 95%, 96%, 97%, 98%, or 99% identical to a RNA sequence encoded by a DNA sequence defined by a set of genomic coordinates shown in Table 21 (human genome) or Table 22 (mouse genome). In some embodiments, the at least one NFKBIA-targeting nucleic acid molecule binds to a target RNA sequence that is 100% identical to a RNA sequence encoded by a DNA sequence defined by a set of genomic coordinates shown in Table 21 or Table 22. In some embodiments, the at least one NFKBIA-targeting nucleic acid molecule binds to a target RNA sequence that is at least 95%, 96%, 97%, 98%, or 99% identical to an RNA sequence encoded by one of SEQ ID NOs: 845-875. In some embodiments, the at least one NFKBIA-targeting nucleic acid molecule binds to a human target RNA sequence that is at least 95%, 96%, 97%, 98%, or 99% identical to a human RNA sequence encoded by one of SEQ ID NOs: 845-856. In some embodiments, the at least one NFKBIA-targeting nucleic acid molecule binds to a target RNA sequence that is 100% identical to an RNA sequence encoded by one of SEQ ID NOs: 845-875. In some embodiments, the at least one NFKBIA-targeting nucleic acid molecule binds to a human target RNA sequence that is 100% identical to a human RNA sequence encoded by one of SEQ ID NOs: 845-856.

In some embodiments, the at least one NFKBIA-targeting nucleic acid molecule is a NFKBIA-targeting shRNA or siRNA molecule. In some embodiments, the at least one NFKBIA-targeting shRNA or siRNA molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one NFKBIA-targeting shRNA or siRNA molecule binds to a target RNA sequence that is 100% identical to a RNA sequence encoded by a DNA sequence defined by a set of genomic coordinates shown in Table 21 or Table 22. In some embodiments, the at least one NFKBIA-targeting shRNA or siRNA molecule binds to a target RNA sequence that is at least 95%, 96%, 97%, 98%, or 99% identical to an RNA sequence encoded by one of SEQ ID NOs: 845-875. In some embodiments, the at least one NFKBIA-targeting shRNA or siRNA molecule binds to a human target RNA sequence that is at least 95%, 96%, 97%, 98%, or 99% identical to a human RNA sequence encoded by one of SEQ ID NOs: 845-856. In some embodiments, the at least one NFKBIA-targeting shRNA or siRNA molecule binds to a target RNA sequence that is 100% identical to an RNA sequence encoded by one of SEQ ID NOs: 845-875. In some embodiments, the at least one NFKBIA-targeting shRNA or siRNA molecule binds to a human target RNA sequence that is 100% identical to a human RNA sequence encoded by one of SEQ ID NOs: 845-856.

TABLE 21
NFKBIA Human Genome Coordinates

| Target | Coordinates |
|---|---|
| NFKBIA | chr14: 35404635-35404654 |
| NFKBIA | chr14: 35402653-35402672 |
| NFKBIA | chr14: 35402494-35402513 |
| NFKBIA | chr14: 35404445-35404464 |
| NFKBIA | chr14: 35403152-35403171 |
| NFKBIA | chr14: 35403258-35403277 |
| NFKBIA | chr14: 35404463-35404482 |
| NFKBIA | chr14: 35403202-35403221 |
| NFKBIA | chr14: 35404411-35404430 |
| NFKBIA | chr14: 35402666-35402685 |
| NFKBIA | chr14: 35403330-35403349 |
| NFKBIA | chr14: 35403695-35403714 |

TABLE 22
Nfkbia Mouse Genome Coordinates

| Target | Coordinates |
|---|---|
| Nfkbia | chr12: 55491236-55491255 |
| Nfkbia | chr12: 55491172-55491191 |
| Nfkbia | chr12: 55491206-55491225 |
| Nfkbia | chr12: 55490633-55490652 |
| Nfkbia | chr12: 55491112-55491131 |
| Nfkbia | chr12: 55490800-55490819 |
| Nfkbia | chr12: 55490821-55490840 |
| Nfkbia | chr12: 55490526-55490545 |
| Nfkbia | chr12: 55491657-55491676 |
| Nfkbia | chr12: 55491177-55491196 |
| Nfkbia | chr12: 55491675-55491694 |
| Nfkbia | chr12: 55490773-55490792 |
| Nfkbia | chr12: 55490809-55490828 |
| Nfkbia | chr12: 55491735-55491754 |
| Nfkbia | chr12: 55490571-55490590 |
| Nfkbia | chr12: 55490588-55490607 |
| Nfkbia | chr12: 55491715-55491734 |
| Nfkbia | chr12: 55492316-55492335 |
| Nfkbia | chr12: 55491207-55491226 |

In some embodiments, the at least one SOCS1-, PTPN2-, ZC3H12A-, CBLB-, RC3H1- or NFKBIA-targeting siRNA molecule or shRNA molecule is obtained from commercial suppliers such as Sigma Aldrich®, Dharmacon®, ThermoFisher®, and the like. In some embodiments, the at least one SOCS1-, PTPN2-, or ZC3H12A-targeting siRNA molecule is one shown in Table 23. In some embodiments, the at least one SOCS1-, PTPN2-, or ZC3H12A-targeting shRNA molecule is one shown in Table 24.

TABLE 23
Exemplary SOCS1, PTPN2, ZC3H12A, RC3H1 and NFKBIA siRNAs

| Target Gene | siRNA construct |
|---|---|
| SOCS1 | MISSION ® esiRNA targeting mouse Socs1 (SigmaAldrich# EMU203261) |
| | Rosetta Predictions human (SigmaAldrich# NM_003745) |
| | Rosetta Predictions murine (SigmaAldrich# NM_009896) |
| PTPN2 | MISSION ® esiRNA human PTPN2 (esiRNA1) (SigmaAldrich# EHU113971) |
| | human Rosetta Predictions (SigmaAldrich# NM_002828) |
| | human Rosetta Predictions (SigmaAldrich# NM_080422) |
| | human Rosetta Predictions (SigmaAldrich# NM_080423) |
| | murine Rosetta Predictions (SigmaAldrich# NM_001127177) |
| ZC3H12A | MISSION ® esiRNA targeting human ZC3H12A (esiRNA1) |
| | (SigmaAldrich# EHU009491) |
| | MISSION ® esiRNA targeting mouse Zc3h12a (esiRNA1) |
| | (SigmaAldrich# EMU048551) |
| | Rosetta Predictions human (SigmaAldrich# NM_025079) |
| | Rosetta Predictions mouse (SigmaAldrich# NM_153159) |
| RC3H1 | MISSION ® esiRNA targeting mouse Cyth4 (SigmaAldrich# EHU032691) |
| NFKBIA | MISSION ® esiRNA targeting mouse Ephb4 (SigmaAldrich# EMU043721) |

TABLE 24
Exemplary SOCS1, PTPN2, ZC3H12A, RC3H1, and NFKBIA shRNAs

| Target Gene | shRNA construct |
|---|---|
| SOCS1 | MISSION ® shRNA Plasmid DNA human (SigmaAldrich# |
| | SHCLND-NM_003745) |
| | MISSION ® shRNA Plasmid DNA murine (SigmaAldrich# |
| | SHCLND-NM_009896) |
| PTPN2 | MISSION ® shRNA Plasmid human (SigmaAldrich# SHCLND-NM_002827) |
| | MISSION ® shRNA Plasmid murine (SigmaAldrich# SHCLND-NM_011201) |
| ZC3H12A | MISSION ® shRNA Plasmid DNA human (SigmaAldrich# |
| | SHCLND-NM_025079) |
| | MISSION ® shRNA Plasmid DNA mouse (SigmaAldrich# |
| | SHCLND-NM_153159) |
| RC3H1 | MISSION ® shRNA Plasmid DNA human (SigmaAldrich# |
| | SHCLND-NM_172071) |
| NFKBIA | MISSION ® shRNA Plasmid DNA human (SigmaAldrich# |
| | SHCLND-NM_020529) |

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a PTPN2-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one SOCS1-targeting nucleic acid molecule binds to a target RNA sequence that is 100% identical to a RNA sequence encoded by a DNA sequence defined by a set of genomic coordinates shown in Table 4 or Table 5 and the at least one PTPN2-targeting nucleic acid molecule binds to a target RNA sequence that is 100% identical to a RNA sequence encoded by a DNA sequence defined by a set of genomic coordinates shown in Table 9 or Table 10.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 23-200 or 23-55 and the at least one PTPN2-targeting nucleic acid molecule binds to a target RNA sequence that is at least 95%, 96%, 97%, 98%, or 99% identical to an RNA sequence encoded by one of SEQ ID NOs: 201-327. In some embodiments, the at least one SOCS1-targeting nucleic acid molecule binds to a target RNA sequence that is 100% identical to an RNA sequence encoded by one of SEQ ID NOs: 23-200 or 23-55 and the at least one PTPN2-targeting nucleic acid molecule binds to a target RNA sequence that is 100% identical to an RNA sequence encoded by one of SEQ ID NOs: 201-327.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a PTPN2-targeting siRNA or shRNA molecule. In some embodiments, the at least one SOCS1-targeting nucleic acid molecule is an siRNA or an shRNA molecule and at least one PTPN2-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one PTPN2-targeting siRNA or shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one PTPN2-targeting siRNA or shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one SOCS1-targeting siRNA or shRNA molecule binds to a target RNA sequence that is 100% identical to a RNA sequence encoded by a DNA sequence defined by a set of genomic coordinates shown in Table 4 or Table 5 and the at least one PTPN2-targeting siRNA or shRNA molecule binds to a target RNA sequence that is 100% identical to a RNA sequence encoded by a DNA sequence defined by a set of genomic coordinates shown in Table 9 or Table 10.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 23-200 or 23-55 and the at least one PTPN2-targeting siRNA or shRNA molecule binds to a target RNA sequence that is at least 95%, 96%, 97%, 98%, or 99% identical to an RNA sequence encoded by one of SEQ ID NOs: 201-327. In some embodiments, the at least one SOCS1-targeting siRNA or shRNA molecule binds to a target RNA sequence that is 100% identical to an RNA sequence encoded by one of SEQ ID NOs: 23-200 or 23-55 and the at least one PTPN2-targeting siRNA or shRNA molecule binds to a target RNA sequence that is 100% identical to an RNA sequence encoded by one of SEQ ID NOs: 201-327.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a ZC3H12A-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one SOCS1-targeting nucleic acid molecule binds to a target RNA sequence that is 100% identical to a RNA sequence encoded by a DNA sequence defined by a set of genomic coordinates shown in Table 4 or Table 5 and the at least one ZC3H12A-targeting nucleic acid molecule binds to a target RNA sequence that is 100% identical to a RNA sequence encoded by a DNA sequence defined by a set of genomic coordinates shown in Table 11 or Table 12.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 23-200 or 23-55 and the at least one ZC3H12A-targeting nucleic acid molecule binds to a target RNA sequence that is at least 95%, 96%, 97%, 98%, or 99% identical to an RNA sequence encoded by one of SEQ ID NOs: 331-797 or 331-337. In some embodiments, the at least one SOCS1-targeting nucleic acid molecule binds to a target RNA sequence that is 100% identical to an RNA sequence encoded by one of SEQ ID NOs: 23-200 or 23-55 and the at least one ZC3H12A-targeting nucleic acid molecule binds to a target RNA sequence that is 100% identical to an RNA sequence encoded by one of SEQ ID NOs: 331-797 or 331-337.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a ZC3H12A-targeting siRNA or shRNA molecule. In some embodiments, the at least one SOCS1-targeting nucleic acid molecule is an siRNA or an shRNA molecule and at least one ZC3H12A-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one ZC3H12A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one ZC3H12A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one ZC3H12A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one SOCS1-targeting siRNA or shRNA molecule binds to a target RNA sequence that is 100% identical to a RNA sequence encoded by a DNA sequence defined by a set of genomic coordinates shown in Table 4 or Table 5 and the at least one ZC3H12A-targeting siRNA or shRNA molecule binds to a target RNA sequence that is 100% identical to a RNA sequence encoded by a DNA sequence defined by a set of genomic coordinates shown in Table 11 or Table 12.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 23-200 or 23-55 and the at least one ZC3H12A-targeting siRNA or shRNA molecule binds to a target RNA sequence that is at least 95%, 96%, 97%, 98%, or 99% identical to an RNA sequence encoded by one of SEQ ID NOs: 331-797 or 331-337. In some embodiments, the at least one SOCS1-targeting siRNA or shRNA molecule binds to a target RNA sequence that is 100% identical to an RNA sequence encoded by one of SEQ ID NOs: 23-200 or 23-55 and the at least one ZC3H12A-targeting siRNA or shRNA molecule binds to a target RNA sequence that is 100% identical to an RNA sequence encoded by one of SEQ ID NOs: 331-797 or 331-337.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a PTPN2-targeting nucleic acid molecule and at least one nucleic acid molecule is a ZC3H12A-targeting nucleic acid molecule. In some embodiments, the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4) and the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 9 or Table 10 and the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one PTPN2-targeting nucleic acid molecule binds to a target RNA sequence that is 100% identical to a RNA sequence encoded by a DNA sequence defined by a set of genomic coordinates shown in Table 9 or Table 10 and the at least one ZC3H12A-targeting nucleic acid molecule binds to a target RNA sequence that is 100% identical to a RNA sequence encoded by a DNA sequence defined by a set of genomic coordinates shown in Table 11 or Table 12.

In some embodiments, the at least one PTPN2-targeting nucleic acid molecule binds to a target RNA sequence that is at least 95%, 96%, 97%, 98%, or 99% identical to an RNA sequence encoded by one of SEQ ID NOs: 201-327 and the at least one ZC3H12A-targeting nucleic acid molecule binds to a target RNA sequence that is at least 95%, 96%, 97%, 98%, or 99% identical to an RNA sequence encoded by one of SEQ ID NOs: 331-797 or 331-337. In some embodiments, the at least one PTPN2-targeting nucleic acid molecule binds to a target RNA sequence that is 100% identical to an RNA sequence encoded by one of SEQ ID NOs: 201-327 and the at least one ZC3H12A-targeting nucleic acid molecule binds to a target RNA sequence that is 100% identical to an RNA sequence encoded by one of SEQ ID NOs: 331-797 or 331-337.

In some embodiments, the nucleic acid-based gene-regulating system comprises at least two siRNA or shRNA molecules, wherein at least one siRNA or shRNA molecule is a PTPN2-targeting siRNA or shRNA molecule and at least one siRNA or shRNA molecule is a ZC3H12A-targeting siRNA or shRNA molecule. In some embodiments, the at least one PTPN2-targeting siRNA or an shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4) and the at least one ZC3H12A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one PTPN2-targeting siRNA or an shRNA molecule binds to a target RNA sequence that is 100% identical to an RNA sequence encoded by the PTPN2 gene (SEQ ID NO: 3) or the Ptpn2 gene (SEQ ID NO: 4) and the at least one ZC3H12A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 9 or Table 10 and the at least one ZC3H12A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one PTPN2-targeting siRNA or shRNA molecule binds to a target RNA sequence that is 100% identical to a RNA sequence encoded by a DNA sequence defined by a set of genomic coordinates shown in Table 9 or Table 10 and the at least one ZC3H12A-targeting siRNA or shRNA molecule binds to a target RNA sequence that is 100% identical to a RNA sequence encoded by a DNA sequence defined by a set of genomic coordinates shown in Table 11 or Table 12.

In some embodiments, the at least one PTPN2-targeting siRNA or shRNA molecule binds to a target RNA sequence that is at least 95%, 96%, 97%, 98%, or 99% identical to an RNA sequence encoded by one of SEQ ID NOs: 201-327 and the at least one ZC3H12A-targeting siRNA or shRNA molecule binds to a target RNA sequence that is at least 95%, 96%, 97%, 98%, or 99% identical to an RNA sequence encoded by one of SEQ ID NOs: 331-797 or 331-337. In some embodiments, the at least one PTPN2-targeting siRNA or shRNA molecule binds to a target RNA sequence that is 100% identical to an RNA sequence encoded by one of SEQ ID NOs: 201-327 and the at least one ZC3H12A-targeting siRNA or shRNA molecule binds to a target RNA sequence that is 100% identical to an RNA sequence encoded by one of SEQ ID NOs: 331-797 or 331-337.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a CBLB-targeting nucleic acid molecule and at least one nucleic acid molecule is a PTPN2-targeting nucleic acid molecule. In some embodiments, the at least one CBLB-targeting nucleic acid molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one CBLB-targeting nucleic acid molecule binds to a target RNA sequence that is 100% identical to an RNA sequence encoded by the CBLB gene (SEQ ID NO: 7) or the Cblb gene (SEQ ID NO: 8) and the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one CBLB-targeting nucleic acid molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one CBLB-targeting nucleic acid molecule binds to a target RNA sequence that is 100% identical to a RNA sequence encoded by a DNA sequence defined by a set of genomic coordinates shown in Table 17 or Table 18 and the at least one PTPN2-targeting nucleic acid molecule binds to a target RNA sequence that is 100% identical to a RNA sequence encoded by a DNA sequence defined by a set of genomic coordinates shown in Table 9 or Table 10.

In some embodiments, the at least one CBLB-targeting nucleic acid molecule binds to a target RNA sequence that is at least 95%, 96%, 97%, 98%, or 99% identical to an RNA sequence encoded by one of SEQ ID NOs: 798-823 and the at least one PTPN2-targeting nucleic acid molecule binds to a target RNA sequence that is at least 95%, 96%, 97%, 98%, or 99% identical to an RNA sequence encoded by one of SEQ ID NOs: 201-327. In some embodiments, the at least one CBLB-targeting nucleic acid molecule binds to a target RNA sequence that is 100% identical to an RNA sequence encoded by one of SEQ ID NOs: 798-823 and the at least one PTPN2-targeting nucleic acid molecule binds to a target RNA sequence that is 100% identical to an RNA sequence encoded by one of SEQ ID NOs: 201-327.

In some embodiments, the nucleic acid-based gene-regulating system comprises at least two siRNA or shRNA molecules, wherein at least one siRNA or shRNA molecule is a CBLB-targeting siRNA or shRNA molecule and at least one siRNA or shRNA molecule is a PTPN2-targeting siRNA or shRNA molecule. In some embodiments, the at least one CBLB-targeting nucleic acid molecule is an siRNA or an shRNA molecule and at least one PTPN2-targeting nucleic acid molecule is an siRNA or shRNA molecule. In some embodiments, the at least one CBLB-targeting siRNA or an shRNA molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one PTPN2-targeting siRNA or shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one CBLB-targeting siRNA or an shRNA molecule binds to a target RNA sequence that is 100% identical to an RNA sequence encoded by the CBLB gene (SEQ ID NO: 7) or the Cblb gene (SEQ ID NO: 8) and the at least one PTPN2-targeting siRNA or shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one CBLB-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one CBLB-targeting siRNA or shRNA molecule binds to a target RNA sequence that is 100% identical to a RNA sequence encoded by a DNA sequence defined by a set of genomic coordinates shown in Table 17 or Table 18 and the at least one PTPN2-targeting siRNA or shRNA molecule binds to a target RNA sequence that is 100% identical to a RNA sequence encoded by a DNA sequence defined by a set of genomic coordinates shown in Table 9 or Table 10.

In some embodiments, the at least one CBLB-targeting siRNA or shRNA molecule binds to a target RNA sequence that is at least 95%, 96%, 97%, 98%, or 99% identical to an RNA sequence encoded by one of SEQ ID NOs: 798-823 and the at least one PTPN2-targeting siRNA or shRNA molecule binds to a target RNA sequence that is at least 95%, 96%, 97%, 98%, or 99% identical to an RNA sequence encoded by one of SEQ ID NOs: 201-327. In some embodiments, the at least one CBLB-targeting siRNA or shRNA molecule binds to a target RNA sequence that is 100% identical to an RNA sequence encoded by one of SEQ ID NOs: 798-823 and the at least one PTPN2-targeting siRNA or shRNA molecule binds to a target RNA sequence that is 100% identical to an RNA sequence encoded by one of SEQ ID NOs: 201-327.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a CBLB-targeting nucleic acid molecule and at least one nucleic acid molecule is a ZC3H12A-targeting nucleic acid molecule. In some embodiments, the at least one CBLB-targeting nucleic acid molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one CBLB-targeting nucleic acid molecule binds to a target RNA sequence that is 100% identical to an RNA sequence encoded by the CBLB gene (SEQ ID NO: 7) or the Cblb gene (SEQ ID NO: 8) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one CBLB-targeting nucleic acid molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one CBLB-targeting nucleic acid molecule binds to a target RNA sequence that is 100% identical to a RNA sequence encoded by a DNA sequence defined by a set of genomic coordinates shown in Table 17 or Table 18 and the at least one ZC3H12A-targeting nucleic acid molecule binds to a target RNA sequence that is 100% identical to a RNA sequence encoded by a DNA sequence defined by a set of genomic coordinates shown in Table 11 or Table 12.

In some embodiments, the at least one CBLB-targeting nucleic acid molecule binds to a target RNA sequence that is at least 95%, 96%, 97%, 98%, or 99% identical to an RNA sequence encoded by one of SEQ ID NOs: 798-823 and the at least one ZC3H12A-targeting nucleic acid molecule binds to a target RNA sequence that is at least 95%, 96%, 97%, 98%, or 99% identical to an RNA sequence encoded by one of SEQ ID NOs: 331-797 or 331-337. In some embodiments, the at least one CBLB-targeting nucleic acid molecule binds to a target RNA sequence that is 100% identical to an RNA sequence encoded by one of SEQ ID NOs: 798-823 and the at least one ZC3H12A-targeting nucleic acid molecule binds to a target RNA sequence that is 100% identical to an RNA sequence encoded by one of SEQ ID NOs: 331-797 or 331-337.

In some embodiments, the nucleic acid-based gene-regulating system comprises at least two siRNA or shRNA molecules, wherein at least one siRNA or shRNA molecule is a CBLB-targeting siRNA or shRNA molecule and at least one siRNA or shRNA molecule is a ZC3H12A-targeting siRNA or shRNA molecule. In some embodiments, the at least one CBLB-targeting nucleic acid molecule is an siRNA or an shRNA molecule and at least one ZC3H12A-targeting nucleic acid molecule is an siRNA or shRNA molecule. In some embodiments, the at least one CBLB-targeting siRNA or an shRNA molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one ZC3H12A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one CBLB-targeting siRNA or an shRNA molecule binds to a target RNA sequence that is 100% identical to an RNA sequence encoded by the CBLB gene (SEQ ID NO: 7) or the Cblb gene (SEQ ID NO: 8) and the at least one ZC3H12A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one CBLB-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one ZC3H12A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one CBLB-targeting siRNA or shRNA molecule binds to a target RNA sequence that is 100% identical to a RNA sequence encoded by a DNA sequence defined by a set of genomic coordinates shown in Table 17 or Table 18 and the at least one ZC3H12A-targeting siRNA or shRNA molecule binds to a target RNA sequence that is 100% identical to a RNA sequence encoded by a DNA sequence defined by a set of genomic coordinates shown in Table 11 or Table 12.

In some embodiments, the at least one CBLB-targeting siRNA or shRNA molecule binds to a target RNA sequence that is at least 95%, 96%, 97%, 98%, or 99% identical to an RNA sequence encoded by one of SEQ ID NOs: 798-823 and the at least one ZC3H12A-targeting siRNA or shRNA molecule binds to a target RNA sequence that is at least 95%, 96%, 97%, 98%, or 99% identical to an RNA sequence encoded by one of SEQ ID NOs: 331-797 or 331-337. In some embodiments, the at least one CBLB-targeting siRNA or shRNA molecule binds to a target RNA sequence that is 100% identical to an RNA sequence encoded by one of SEQ ID NOs: 798-823 and the at least one ZC3H12A-targeting siRNA or shRNA molecule binds to a target RNA sequence that is 100% identical to an RNA sequence encoded by one of SEQ ID NOs: 331-797 or 331-337.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a CBLB-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one CBLB-targeting nucleic acid molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one CBLB-targeting nucleic acid molecule binds to a target RNA sequence that is 100% identical to an RNA sequence encoded by the CBLB gene (SEQ ID NO: 7) or the Cblb gene (SEQ ID NO: 8).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one CBLB-targeting nucleic acid molecule binds to a target RNA sequence that is at least 95%, 96%, 97%, 98%, or 99% identical to a RNA sequence encoded by a DNA sequence defined by a set of genomic coordinates shown in Table 17 or Table 18. In some embodiments, the at least one SOCS1-targeting nucleic acid molecule binds to a target RNA sequence that is 100% identical to a RNA sequence encoded by a DNA sequence defined by a set of genomic coordinates shown in Table 4 or Table 5 and the at least one CBLB-targeting nucleic acid molecule binds to a target RNA sequence that is 100% identical to a RNA sequence encoded by a DNA sequence defined by a set of genomic coordinates shown in Table 17 or Table 18.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 23-200 or 23-55 and the at least one CBLB-targeting nucleic acid molecule binds to a target RNA sequence that is at least 95%, 96%, 97%, 98%, or 99% identical to an RNA sequence encoded by one of SEQ ID NOs: 798-823. In some embodiments, the at least one SOCS1-targeting nucleic acid molecule binds to a target RNA sequence that is 100% identical to an RNA sequence encoded by one of SEQ ID NOs: 23-200 or 23-55 and the at least one CBLB-targeting nucleic acid molecule binds to a target RNA sequence that is 100% identical to an RNA sequence encoded by one of SEQ ID NOs: 798-823.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a CBLB-targeting siRNA or shRNA molecule. In some embodiments, the at least one SOCS1-targeting nucleic acid molecule is an siRNA or an shRNA molecule and at least one CBLB-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one CBLB-targeting siRNA or shRNA molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one CBLB-targeting siRNA or shRNA molecule binds to a target RNA sequence that is 100% identical to an RNA sequence encoded by the CBLB gene (SEQ ID NO: 7) or the Cblb gene (SEQ ID NO: 8).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one CBLB-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 17 or Table 18. In some embodiments, the at least one SOCS1-targeting siRNA or shRNA molecule binds to a target RNA sequence that is 100% identical to a RNA sequence encoded by a DNA sequence defined by a set of genomic coordinates shown in Table 4 or Table 5 and the at least one CBLB-targeting siRNA or shRNA molecule binds to a target RNA sequence that is 100% identical to a RNA sequence encoded by a DNA sequence defined by a set of genomic coordinates shown in Table 17 or Table 18.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 23-200 or 23-55 and the at least one CBLB-targeting siRNA or shRNA molecule binds to a target RNA sequence that is at least 95%, 96%, 97%, 98%, or 99% identical to an RNA sequence encoded by one of SEQ ID NOs: 798-823. In some embodiments, the at least one SOCS1-targeting siRNA or shRNA molecule binds to a target RNA sequence that is 100% identical to an RNA sequence encoded by one of SEQ ID NOs: 23-200 or 23-55 and the at least one CBLB-targeting siRNA or shRNA molecule binds to a target RNA sequence that is 100% identical to an RNA sequence encoded by one of SEQ ID NOs: 798-823.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a RC3H1-targeting nucleic acid molecule and at least one nucleic acid molecule is a PTPN2-targeting nucleic acid molecule. In some embodiments, the at least one RC3H1-targeting nucleic acid molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10) and the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one RC3H1-targeting nucleic acid molecule binds to a target RNA sequence that is 100% identical to an RNA sequence encoded by the RC3H1 gene (SEQ ID NO: 9) or the Rc3h1 gene (SEQ ID NO: 10) and the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one RC3H1-targeting nucleic acid molecule binds to a target RNA sequence that is at least 95%, 96%, 97%, 98%, or 99% identical to a RNA sequence encoded by a DNA sequence defined by a set of genomic coordinates shown in Table 19 or Table 20 and the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one RC3H1-targeting nucleic acid molecule binds to a target RNA sequence that is 100% identical to a RNA sequence encoded by a DNA sequence defined by a set of genomic coordinates shown in Table 19 or Table 20 and the at least one PTPN2-targeting nucleic acid molecule binds to a target RNA sequence that is 100% identical to a RNA sequence encoded by a DNA sequence defined by a set of genomic coordinates shown in Table 9 or Table 10.

In some embodiments, the at least one RC3H1-targeting nucleic acid molecule binds to a target RNA sequence that is at least 95%, 96%, 97%, 98%, or 99% identical to an RNA sequence encoded by one of SEQ ID NOs: 824-844 and the at least one PTPN2-targeting nucleic acid molecule binds to a target RNA sequence that is at least 95%, 96%, 97%, 98%, or 99% identical to an RNA sequence encoded by one of SEQ ID NOs: 201-327. In some embodiments, the at least one RC3H1-targeting nucleic acid molecule binds to a target RNA sequence that is 100% identical to an RNA sequence encoded by one of SEQ ID NOs: 824-844 and the at least one PTPN2-targeting nucleic acid molecule binds to a target RNA sequence that is 100% identical to an RNA sequence encoded by one of SEQ ID NOs: 201-327.

In some embodiments, the nucleic acid-based gene-regulating system comprises at least two siRNA or shRNA molecules, wherein at least one siRNA or shRNA molecule is a RC3H1-targeting siRNA or shRNA molecule and at least one siRNA or shRNA molecule is a PTPN2-targeting siRNA or shRNA molecule. In some embodiments, the at least one RC3H1-targeting nucleic acid molecule is an siRNA or an shRNA molecule and at least one PTPN2-targeting nucleic acid molecule is an siRNA or shRNA molecule. In some embodiments, the at least one RC3H1-targeting siRNA or an shRNA molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10) and the at least one PTPN2-targeting siRNA or shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one RC3H1-targeting siRNA or an shRNA molecule binds to a target RNA sequence that is 100% identical to an RNA sequence encoded by the RC3H1 gene (SEQ ID NO: 9) or the Rc3h1 gene (SEQ ID NO: 10) and the at least one PTPN2-targeting siRNA or shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one RC3H1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 19 or Table 20 and the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one RC3H1-targeting siRNA or shRNA molecule binds to a target RNA sequence that is 100% identical to a RNA sequence encoded by a DNA sequence defined by a set of genomic coordinates shown in Table 19 or Table 20 and the at least one PTPN2-targeting siRNA or shRNA molecule binds to a target RNA sequence that is 100% identical to a RNA sequence encoded by a DNA sequence defined by a set of genomic coordinates shown in Table 9 or Table 10.

In some embodiments, the at least one RC3H1-targeting siRNA or shRNA molecule binds to a target RNA sequence that is at least 95%, 96%, 97%, 98%, or 99% identical to an RNA sequence encoded by one of SEQ ID NOs: 824-844 and the at least one PTPN2-targeting siRNA or shRNA molecule binds to a target RNA sequence that is at least 95%, 96%, 97%, 98%, or 99% identical to an RNA sequence encoded by one of SEQ ID NOs: 201-327. In some embodiments, the at least one RC3H1-targeting siRNA or shRNA molecule binds to a target RNA sequence that is 100% identical to an RNA sequence encoded by one of SEQ ID NOs: 824-844 and the at least one PTPN2-targeting siRNA or shRNA molecule binds to a target RNA sequence that is 100% identical to an RNA sequence encoded by one of SEQ ID NOs: 201-327.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a RC3H1-targeting nucleic acid molecule and at least one nucleic acid molecule is a ZC3H12A-targeting nucleic acid molecule. In some embodiments, the at least one RC3H1-targeting nucleic acid molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10) and the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one RC3H1-targeting nucleic acid molecule binds to a target RNA sequence that is 100% identical to an RNA sequence encoded by the RC3H1 gene (SEQ ID NO: 9) or the Rc3h1 gene (SEQ ID NO: 10) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one RC3H1-targeting nucleic acid molecule binds to a target RNA sequence that is at least 95%, 96%, 97%, 98%, or 99% identical to a RNA sequence encoded by a DNA sequence defined by a set of genomic coordinates shown in Table 19 or Table 20 and the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one RC3H1-targeting nucleic acid molecule binds to a target RNA sequence that is 100% identical to a RNA sequence encoded by a DNA sequence defined by a set of genomic coordinates shown in Table 19 or Table 20 and the at least one ZC3H12A-targeting nucleic acid molecule binds to a target RNA sequence that is 100% identical to a RNA sequence encoded by a DNA sequence defined by a set of genomic coordinates shown in Table 11 or Table 12.

In some embodiments, the at least one RC3H1-targeting nucleic acid molecule binds to a target RNA sequence that is at least 95%, 96%, 97%, 98%, or 99% identical to an RNA sequence encoded by one of SEQ ID NOs: 824-844 and the at least one ZC3H12A-targeting nucleic acid molecule binds to a target RNA sequence that is at least 95%, 96%, 97%, 98%, or 99% identical to an RNA sequence encoded by one of SEQ ID NOs: 331-797 or 331-337. In some embodiments, the at least one RC3H1-targeting nucleic acid molecule binds to a target RNA sequence that is 100% identical to an RNA sequence encoded by one of SEQ ID NOs: 824-844 and the at least one ZC3H12A-targeting nucleic acid molecule binds to a target RNA sequence that is 100% identical to an RNA sequence encoded by one of SEQ ID NOs: 331-797 or 331-337.

In some embodiments, the nucleic acid-based gene-regulating system comprises at least two siRNA or shRNA molecules, wherein at least one siRNA or shRNA molecule is a RC3H1-targeting siRNA or shRNA molecule and at least one siRNA or shRNA molecule is a ZC3H12A-targeting siRNA or shRNA molecule. In some embodiments, the at least one RC3H1-targeting nucleic acid molecule is an siRNA or an shRNA molecule and at least one ZC3H12A-targeting nucleic acid molecule is an siRNA or shRNA molecule. In some embodiments, the at least one RC3H1-targeting siRNA or an shRNA molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10) and the at least one ZC3H12A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one RC3H1-targeting siRNA or an shRNA molecule binds to a target RNA sequence that is 100% identical to an RNA sequence encoded by the RC3H1 gene (SEQ ID NO: 9) or the Rc3h1 gene (SEQ ID NO: 10) and the at least one ZC3H12A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one RC3H1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 19 or Table 20 and the at least one ZC3H12A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one RC3H1-targeting siRNA or shRNA molecule binds to a target RNA sequence that is 100% identical to a RNA sequence encoded by a DNA sequence defined by a set of genomic coordinates shown in Table 19 or Table 20 and the at least one ZC3H12A-targeting siRNA or shRNA molecule binds to a target RNA sequence that is 100% identical to a RNA sequence encoded by a DNA sequence defined by a set of genomic coordinates shown in Table 11 or Table 12.

In some embodiments, the at least one RC3H1-targeting siRNA or shRNA molecule binds to a target RNA sequence that is at least 95%, 96%, 97%, 98%, or 99% identical to an RNA sequence encoded by one of SEQ ID NOs: 824-844 and the at least one ZC3H12A-targeting siRNA or shRNA molecule binds to a target RNA sequence that is at least 95%, 96%, 97%, 98%, or 99% identical to an RNA sequence encoded by one of SEQ ID NOs: 331-797 or 331-337. In some embodiments, the at least one RC3H1-targeting siRNA or shRNA molecule binds to a target RNA sequence that is 100% identical to an RNA sequence encoded by one of SEQ ID NOs: 824-844 and the at least one ZC3H12A-targeting siRNA or shRNA molecule binds to a target RNA sequence that is 100% identical to an RNA sequence encoded by one of SEQ ID NOs: 331-797 or 331-337.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a RC3H1-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one RC3H1-targeting nucleic acid molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one RC3H1-targeting nucleic acid molecule binds to a target RNA sequence that is 100% identical to an RNA sequence encoded by the RC3H1 gene (SEQ ID NO: 9) or the Rc3h1 gene (SEQ ID NO: 10).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one RC3H1-targeting nucleic acid molecule binds to a target RNA sequence that is at least 95%, 96%, 97%, 98%, or 99% identical to a RNA sequence encoded by a DNA sequence defined by a set of genomic coordinates shown in Table 19 or Table 20. In some embodiments, the at least one SOCS1-targeting nucleic acid molecule binds to a target RNA sequence that is 100% identical to a RNA sequence encoded by a DNA sequence defined by a set of genomic coordinates shown in Table 4 or Table 5 and the at least one RC3H1-targeting nucleic acid molecule binds to a target RNA sequence that is 100% identical to a RNA sequence encoded by a DNA sequence defined by a set of genomic coordinates shown in Table 19 or Table 20.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 23-200 or 23-55 and the at least one RC3H1-targeting nucleic acid molecule binds to a target RNA sequence that is at least 95%, 96%, 97%, 98%, or 99% identical to an RNA sequence encoded by one of SEQ ID NOs: 824-844. In some embodiments, the at least one SOCS1-targeting nucleic acid molecule binds to a target RNA sequence that is 100% identical to an RNA sequence encoded by one of SEQ ID NOs: 23-200 or 23-55 and the at least one RC3H1-targeting nucleic acid molecule binds to a target RNA sequence that is 100% identical to an RNA sequence encoded by one of SEQ ID NOs: 824-844.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a RC3H1-targeting siRNA or shRNA molecule. In some embodiments, the at least one SOCS1-targeting nucleic acid molecule is an siRNA or an shRNA molecule and at least one RC3H1-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one RC3H1-targeting siRNA or shRNA molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one RC3H1-targeting siRNA or shRNA molecule binds to a target RNA sequence that is 100% identical to an RNA sequence encoded by the RC3H1 gene (SEQ ID NO: 9) or the Rc3h1 gene (SEQ ID NO: 10).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one RC3H1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 19 or Table 20. In some embodiments, the at least one SOCS1-targeting siRNA or shRNA molecule binds to a target RNA sequence that is 100% identical to a RNA sequence encoded by a DNA sequence defined by a set of genomic coordinates shown in Table 4 or Table 5 and the at least one RC3H1-targeting siRNA or shRNA molecule binds to a target RNA sequence that is 100% identical to a RNA sequence encoded by a DNA sequence defined by a set of genomic coordinates shown in Table 19 or Table 20.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 23-200 or 23-55 and the at least one RC3H1-targeting siRNA or shRNA molecule binds to a target RNA sequence that is at least 95%, 96%, 97%, 98%, or 99% identical to an RNA sequence encoded by one of SEQ ID NOs: 824-844. In some embodiments, the at least one SOCS1-targeting siRNA or shRNA molecule binds to a target RNA sequence that is 100% identical to an RNA sequence encoded by one of SEQ ID NOs: 23-200 or 23-55 and the at least one RC3H1-targeting siRNA or shRNA molecule binds to a target RNA sequence that is 100% identical to an RNA sequence encoded by one of SEQ ID NOs: 824-844.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a CBLB-targeting nucleic acid molecule and at least one nucleic acid molecule is a RC3H1-targeting nucleic acid molecule. In some embodiments, the at least one CBLB-targeting nucleic acid molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one RC3H1-targeting nucleic acid molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10). In some embodiments, the at least one CBLB-targeting nucleic acid molecule binds to a target RNA sequence that is 100% identical to an RNA sequence encoded by the CBLB gene (SEQ ID NO: 7) or the Cblb gene (SEQ ID NO: 8) and the at least one RC3H1-targeting nucleic acid molecule binds to a target RNA sequence that is 100% identical to an RNA sequence encoded by the RC3H1 gene (SEQ ID NO: 9) or the Rc3h1 gene (SEQ ID NO: 10).

In some embodiments, the at least one CBLB-targeting nucleic acid molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one RC3H1-targeting nucleic acid molecule binds to a target RNA sequence that is at least 95%, 96%, 97%, 98%, or 99% identical to a RNA sequence encoded by a DNA sequence defined by a set of genomic coordinates shown in Table 19 or Table 20. In some embodiments, the at least one CBLB-targeting nucleic acid molecule binds to a target RNA sequence that is 100% identical to a RNA sequence encoded by a DNA sequence defined by a set of genomic coordinates shown in Table 17 or Table 18 and the at least one RC3H1-targeting nucleic acid molecule binds to a target RNA sequence that is 100% identical to a RNA sequence encoded by a DNA sequence defined by a set of genomic coordinates shown in Table 19 or Table 20.

In some embodiments, the at least one CBLB-targeting nucleic acid molecule binds to a target RNA sequence that is at least 95%, 96%, 97%, 98%, or 99% identical to an RNA sequence encoded by one of SEQ ID NOs: 798-823 and the at least one RC3H1-targeting nucleic acid molecule binds to a target RNA sequence that is at least 95%, 96%, 97%, 98%, or 99% identical to an RNA sequence encoded by one of SEQ ID NOs: 824-844. In some embodiments, the at least one CBLB-targeting nucleic acid molecule binds to a target RNA sequence that is 100% identical to an RNA sequence encoded by one of SEQ ID NOs: 798-823 and the at least one RC3H1-targeting nucleic acid molecule binds to a target RNA sequence that is 100% identical to an RNA sequence encoded by one of SEQ ID NOs: 824-844.

In some embodiments, the nucleic acid-based gene-regulating system comprises at least two siRNA or shRNA molecules, wherein at least one siRNA or shRNA molecule is a CBLB-targeting siRNA or shRNA molecule and at least one siRNA or shRNA molecule is a RC3H1-targeting siRNA or shRNA molecule. In some embodiments, the at least one CBLB-targeting nucleic acid molecule is an siRNA or an shRNA molecule and at least one RC3H1-targeting nucleic acid molecule is an siRNA or shRNA molecule. In some embodiments, the at least one CBLB-targeting siRNA or an shRNA molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one RC3H1-targeting siRNA or shRNA molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10). In some embodiments, the at least one CBLB-targeting siRNA or an shRNA molecule binds to a target RNA sequence that is 100% identical to an RNA sequence encoded by the CBLB gene (SEQ ID NO: 7) or the Cblb gene (SEQ ID NO: 8) and the at least one RC3H1-targeting siRNA or shRNA molecule binds to a target RNA sequence that is 100% identical to an RNA sequence encoded by the RC3H1 gene (SEQ ID NO: 9) or the Rc3h1 gene (SEQ ID NO: 10).

In some embodiments, the at least one CBLB-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one RC3H1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 19 or Table 20. In some embodiments, the at least one CBLB-targeting siRNA or shRNA molecule binds to a target RNA sequence that is 100% identical to a RNA sequence encoded by a DNA sequence defined by a set of genomic coordinates shown in Table 17 or Table 18 and the at least one RC3H1-targeting siRNA or shRNA molecule binds to a target RNA sequence that is 100% identical to a RNA sequence encoded by a DNA sequence defined by a set of genomic coordinates shown in Table 19 or Table 20.

In some embodiments, the at least one CBLB-targeting siRNA or shRNA molecule binds to a target RNA sequence that is at least 95%, 96%, 97%, 98%, or 99% identical to an RNA sequence encoded by one of SEQ ID NOs: 798-823 and the at least one RC3H1-targeting siRNA or shRNA molecule binds to a target RNA sequence that is at least 95%, 96%, 97%, 98%, or 99% identical to an RNA sequence encoded by one of SEQ ID NOs: 824-844. In some embodiments, the at least one CBLB-targeting siRNA or shRNA molecule binds to a target RNA sequence that is 100% identical to an RNA sequence encoded by one of SEQ ID NOs: 798-823 and the at least one RC3H1-targeting siRNA or shRNA molecule binds to a target RNA sequence that is 100% identical to an RNA sequence encoded by one of SEQ ID NOs: 824-844.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a NFKBIA-targeting nucleic acid molecule and at least one nucleic acid molecule is a PTPN2-targeting nucleic acid molecule. In some embodiments, the at least one NFKBIA-targeting nucleic acid molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12) and the at least one PTPN2-targeting nucleic acid molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one NFKBIA-targeting nucleic acid molecule binds to a target RNA sequence that is 100% identical to an RNA sequence encoded by the NFKBIA gene (SEQ ID NO: 11) or the Nfkbia gene (SEQ ID NO: 12) and the at least one PTPN2-targeting nucleic acid molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one NFKBIA-targeting nucleic acid molecule binds to a target RNA sequence that is at least 95%, 96%, 97%, 98%, or 99% identical to a RNA sequence encoded by a DNA sequence defined by a set of genomic coordinates shown in Table 21 or Table 22 and the at least one PTPN2-targeting nucleic acid molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one NFKBIA-targeting nucleic acid molecule binds to a target RNA sequence that is 100% identical to a RNA sequence encoded by a DNA sequence defined by a set of genomic coordinates shown in Table 21 or Table 22 and the at least one PTPN2-targeting nucleic acid molecule binds to a target RNA sequence that is 100% identical to a RNA sequence encoded by a DNA sequence defined by a set of genomic coordinates shown in Table 9 or Table 10.

In some embodiments, the at least one NFKBIA-targeting nucleic acid molecule binds to a target RNA sequence that is at least 95%, 96%, 97%, 98%, or 99% identical to an RNA sequence encoded by one of SEQ ID NOs: 845-875 and the at least one PTPN2-targeting nucleic acid molecule binds to a target RNA sequence that is at least 95%, 96%, 97%, 98%, or 99% identical to an RNA sequence encoded by one of SEQ ID NOs: 201-327. In some embodiments, the at least one NFKBIA-targeting nucleic acid molecule binds to a target RNA sequence that is 100% identical to an RNA sequence encoded by one of SEQ ID NOs: 845-875 and the at least one PTPN2-targeting nucleic acid molecule binds to a target RNA sequence that is 100% identical to an RNA sequence encoded by one of SEQ ID NOs: 201-327.

In some embodiments, the nucleic acid-based gene-regulating system comprises at least two siRNA or shRNA molecules, wherein at least one siRNA or shRNA molecule is a NFKBIA-targeting siRNA or shRNA molecule and at least one siRNA or shRNA molecule is a PTPN2-targeting siRNA or shRNA molecule. In some embodiments, the at least one NFKBIA-targeting nucleic acid molecule is an siRNA or an shRNA molecule and at least one PTPN2-targeting nucleic acid molecule is an siRNA or shRNA molecule. In some embodiments, the at least one NFKBIA-targeting siRNA or an shRNA molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12) and the at least one PTPN2-targeting siRNA or shRNA molecule binds to a target RNA sequence that is at least 95%, 96%, 97%, 98%, or 99% identical to an RNA sequence encoded by the PTPN2 gene (SEQ ID NO: 3) or the Ptpn2 gene (SEQ ID NO: 4). In some embodiments, the at least one NFKBIA-targeting siRNA or an shRNA molecule binds to a target RNA sequence that is 100% identical to an RNA sequence encoded by the NFKBIA gene (SEQ ID NO: 11) or the Nfkbia gene (SEQ ID NO: 12) and the at least one PTPN2-targeting siRNA or shRNA molecule binds to a target RNA sequence that is 100% identical to an RNA sequence encoded by the PTPN2 gene (SEQ ID NO: 3) or the Ptpn2 gene (SEQ ID NO: 4).

In some embodiments, the at least one NFKBIA-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 21 or Table 22 and the at least one PTPN2-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 9 or Table 10. In some embodiments, the at least one NFKBIA-targeting siRNA or shRNA molecule binds to a target RNA sequence that is 100% identical to a RNA sequence encoded by a DNA sequence defined by a set of genomic coordinates shown in Table 21 or Table 22 and the at least one PTPN2-targeting siRNA or shRNA molecule binds to a target RNA sequence that is 100% identical to a RNA sequence encoded by a DNA sequence defined by a set of genomic coordinates shown in Table 9 or Table 10.

In some embodiments, the at least one NFKBIA-targeting siRNA or shRNA molecule binds to a target RNA sequence that is at least 95%, 96%, 97%, 98%, or 99% identical to an RNA sequence encoded by one of SEQ ID NOs: 845-875 and the at least one PTPN2-targeting siRNA or shRNA molecule binds to a target RNA sequence that is at least 95%, 96%, 97%, 98%, or 99% identical to an RNA sequence encoded by one of SEQ ID NOs: 201-327. In some embodiments, the at least one NFKBIA-targeting siRNA or shRNA molecule binds to a target RNA sequence that is 100% identical to an RNA sequence encoded by one of SEQ ID NOs: 845-875 and the at least one PTPN2-targeting siRNA or shRNA molecule binds to a target RNA sequence that is 100% identical to an RNA sequence encoded by one of SEQ ID NOs: 201-327.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a NFKBIA-targeting nucleic acid molecule and at least one nucleic acid molecule is a ZC3H12A-targeting nucleic acid molecule. In some embodiments, the at least one NFKBIA-targeting nucleic acid molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12) and the at least one ZC3H12A-targeting nucleic acid molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one NFKBIA-targeting nucleic acid molecule binds to a target RNA sequence that is 100% identical to an RNA sequence encoded by the NFKBIA gene (SEQ ID NO: 11) or the Nfkbia gene (SEQ ID NO: 12) and the at least one ZC3H12A-targeting nucleic acid molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one NFKBIA-targeting nucleic acid molecule binds to a target RNA sequence that is at least 95%, 96%, 97%, 98%, or 99% identical to a RNA sequence encoded by a DNA sequence defined by a set of genomic coordinates shown in Table 21 or Table 22 and the at least one ZC3H12A-targeting nucleic acid molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one NFKBIA-targeting nucleic acid molecule binds to a target RNA sequence that is 100% identical to a RNA sequence encoded by a DNA sequence defined by a set of genomic coordinates shown in Table 21 or Table 22 and the at least one ZC3H12A-targeting nucleic acid molecule binds to a target RNA sequence that is 100% identical to a RNA sequence encoded by a DNA sequence defined by a set of genomic coordinates shown in Table 11 or Table 12.

In some embodiments, the at least one NFKBIA-targeting nucleic acid molecule binds to a target RNA sequence that is at least 95%, 96%, 97%, 98%, or 99% identical to an RNA sequence encoded by one of SEQ ID NOs: 845-875 and the at least one ZC3H12A-targeting nucleic acid molecule binds to a target RNA sequence that is at least 95%, 96%, 97%, 98%, or 99% identical to an RNA sequence encoded by one of SEQ ID NOs: 331-797 or 331-337. In some embodiments, the at least one NFKBIA-targeting nucleic acid molecule binds to a target RNA sequence that is 100% identical to an RNA sequence encoded by one of SEQ ID NOs: 845-875 and the at least one ZC3H12A-targeting nucleic acid molecule binds to a target RNA sequence that is 100% identical to an RNA sequence encoded by one of SEQ ID NOs: 331-797 or 331-337.

In some embodiments, the nucleic acid-based gene-regulating system comprises at least two siRNA or shRNA molecules, wherein at least one siRNA or shRNA molecule is a NFKBIA-targeting siRNA or shRNA molecule and at least one siRNA or shRNA molecule is a ZC3H12A-targeting siRNA or shRNA molecule. In some embodiments, the at least one NFKBIA-targeting nucleic acid molecule is an siRNA or an shRNA molecule and at least one ZC3H12A-targeting nucleic acid molecule is an siRNA or shRNA molecule. In some embodiments, the at least one NFKBIA-targeting siRNA or an shRNA molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12) and the at least one ZC3H12A-targeting siRNA or shRNA molecule binds to a target RNA sequence that is at least 95%, 96%, 97%, 98%, or 99% identical to an RNA sequence encoded by the ZC3H12A gene (SEQ ID NO: 5) or the Zc3h12a gene (SEQ ID NO: 6). In some embodiments, the at least one NFKBIA-targeting siRNA or an shRNA molecule binds to a target RNA sequence that is 100% identical to an RNA sequence encoded by the NFKBIA gene (SEQ ID NO: 11) or the Nfkbia gene (SEQ ID NO: 12) and the at least one ZC3H12A-targeting siRNA or shRNA molecule binds to a target RNA sequence that is 100% identical to an RNA sequence encoded by the ZC3H12A gene (SEQ ID NO: 5) or the Zc3h12a gene (SEQ ID NO: 6).

In some embodiments, the at least one NFKBIA-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 21 or Table 22 and the at least one ZC3H12A-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 11 or Table 12. In some embodiments, the at least one NFKBIA-targeting siRNA or shRNA molecule binds to a target RNA sequence that is 100% identical to a RNA sequence encoded by a DNA sequence defined by a set of genomic coordinates shown in Table 21 or Table 22 and the at least one ZC3H12A-targeting siRNA or shRNA molecule binds to a target RNA sequence that is 100% identical to a RNA sequence encoded by a DNA sequence defined by a set of genomic coordinates shown in Table 11 or Table 12.

In some embodiments, the at least one NFKBIA-targeting siRNA or shRNA molecule binds to a target RNA sequence that is at least 95%, 96%, 97%, 98%, or 99% identical to an RNA sequence encoded by one of SEQ ID NOs: 845-875 and the at least one ZC3H12A-targeting siRNA or shRNA molecule binds to a target RNA sequence that is at least 95%, 96%, 97%, 98%, or 99% identical to an RNA sequence encoded by one of SEQ ID NOs: 331-797 or 331-337. In some embodiments, the at least one NFKBIA-targeting siRNA or shRNA molecule binds to a target RNA sequence that is 100% identical to an RNA sequence encoded by one of SEQ ID NOs: 845-875 and the at least one ZC3H12A-targeting siRNA or shRNA molecule binds to a target RNA sequence that is 100% identical to an RNA sequence encoded by one of SEQ ID NOs: 331-797 or 331-337.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a SOCS1-targeting nucleic acid molecule and at least one nucleic acid molecule is a NFKBIA-targeting nucleic acid molecule. In some embodiments, the at least one SOCS1-targeting nucleic acid molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one NFKBIA-targeting nucleic acid molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one SOCS1-targeting nucleic acid molecule binds to a target RNA sequence that is 100% identical to an RNA sequence encoded by the SOCS1 gene (SEQ ID NO: 1) or the Socs1 gene (SEQ ID NO: 2) and the at least one NFKBIA-targeting nucleic acid molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one SOCS1-targeting nucleic acid molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one NFKBIA-targeting nucleic acid molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one SOCS1-targeting nucleic acid molecule binds to a target RNA sequence that is 100% identical to a RNA sequence encoded by a DNA sequence defined by a set of genomic coordinates shown in Table 4 or Table 5 and the at least one NFKBIA-targeting nucleic acid molecule binds to a target RNA sequence that is 100% identical to a RNA sequence encoded by a DNA sequence defined by a set of genomic coordinates shown in Table 21 or Table 22.

In some embodiments, the at least one SOCS1-targeting nucleic acid molecule binds to a target RNA sequence that is at least 95%, 96%, 97%, 98%, or 99% identical to an RNA sequence encoded by one of SEQ ID NOs: 23-200 or 23-55 and the at least one NFKBIA-targeting nucleic acid molecule binds to a target RNA sequence that is at least 95%, 96%, 97%, 98%, or 99% identical to an RNA sequence encoded by one of SEQ ID NOs: 845-875. In some embodiments, the at least one SOCS1-targeting nucleic acid molecule binds to a target RNA sequence that is 100% identical to an RNA sequence encoded by one of SEQ ID NOs: 23-200 or 23-55 and the at least one NFKBIA-targeting nucleic acid molecule binds to a target RNA sequence that is 100% identical to an RNA sequence encoded by one of SEQ ID NOs: 845-875.

In some embodiments, the nucleic acid-based gene-regulating system comprises at least two siRNA or shRNA molecules, wherein at least one siRNA or shRNA molecule is a SOCS1-targeting siRNA or shRNA molecule and at least one siRNA or shRNA molecule is a NFKBIA-targeting siRNA or shRNA molecule. In some embodiments, the at least one SOCS1-targeting nucleic acid molecule is an siRNA or an shRNA molecule and at least one NFKBIA-targeting nucleic acid molecule is an siRNA or shRNA molecule. In some embodiments, the at least one SOCS1-targeting siRNA or an shRNA molecule binds to a target RNA sequence that is at least 95%, 96%, 97%, 98%, or 99% identical to an RNA sequence encoded by the SOCS1 gene (SEQ ID NO: 1) or the Socs1 gene (SEQ ID NO: 2) and the at least one NFKBIA-targeting siRNA or shRNA molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one SOCS1-targeting siRNA or an shRNA molecule binds to a target RNA sequence that is 100% identical to an RNA sequence encoded by the SOCS1 gene (SEQ ID NO: 1) or the Socs1 gene (SEQ ID NO: 2) and the at least one NFKBIA-targeting siRNA or shRNA molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one SOCS1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 4 or Table 5 and the at least one NFKBIA-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one SOCS1-targeting siRNA or shRNA molecule binds to a target RNA sequence that is 100% identical to a RNA sequence encoded by a DNA sequence defined by a set of genomic coordinates shown in Table 4 or Table 5 and the at least one NFKBIA-targeting siRNA or shRNA molecule binds to a target RNA sequence that is 100% identical to a RNA sequence encoded by a DNA sequence defined by a set of genomic coordinates shown in Table 21 or Table 22.

In some embodiments, the at least one SOCS1-targeting siRNA or shRNA molecule binds to a target RNA sequence that is at least 95%, 96%, 97%, 98%, or 99% identical to an RNA sequence encoded by one of SEQ ID NOs: 23-200 or 23-55 and the at least one NFKBIA-targeting siRNA or shRNA molecule binds to a target RNA sequence that is at least 95%, 96%, 97%, 98%, or 99% identical to an RNA sequence encoded by one of SEQ ID NOs: 845-875. In some embodiments, the at least one SOCS1-targeting siRNA or shRNA molecule binds to a target RNA sequence that is 100% identical to an RNA sequence encoded by one of SEQ ID NOs: 23-200 or 23-55 and the at least one NFKBIA-targeting siRNA or shRNA molecule binds to a target RNA sequence that is 100% identical to an RNA sequence encoded by one of SEQ ID NOs: 845-875.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a CBLB-targeting nucleic acid molecule and at least one nucleic acid molecule is a NFKBIA-targeting nucleic acid molecule. In some embodiments, the at least one CBLB-targeting nucleic acid molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one NFKBIA-targeting nucleic acid molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one CBLB-targeting nucleic acid molecule binds to a target RNA sequence that is 100% identical to an RNA sequence encoded by the CBLB gene (SEQ ID NO: 7) or the Cblb gene (SEQ ID NO: 8) and the at least one NFKBIA-targeting nucleic acid molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one CBLB-targeting nucleic acid molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one NFKBIA-targeting nucleic acid molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one CBLB-targeting nucleic acid molecule binds to a target RNA sequence that is 100% identical to a RNA sequence encoded by a DNA sequence defined by a set of genomic coordinates shown in Table 17 or Table 18 and the at least one NFKBIA-targeting nucleic acid molecule binds to a target RNA sequence that is 100% identical to a RNA sequence encoded by a DNA sequence defined by a set of genomic coordinates shown in Table 21 or Table 22.

In some embodiments, the at least one CBLB-targeting nucleic acid molecule binds to a target RNA sequence that is at least 95%, 96%, 97%, 98%, or 99% identical to an RNA sequence encoded by one of SEQ ID NOs: 798-823 and the at least one NFKBIA-targeting nucleic acid molecule binds to a target RNA sequence that is at least 95%, 96%, 97%, 98%, or 99% identical to an RNA sequence encoded by one of SEQ ID NOs: 845-875. In some embodiments, the at least one CBLB-targeting nucleic acid molecule binds to a target RNA sequence that is 100% identical to an RNA sequence encoded by one of SEQ ID NOs: 798-823 and the at least one NFKBIA-targeting nucleic acid molecule binds to a target RNA sequence that is 100% identical to an RNA sequence encoded by one of SEQ ID NOs: 845-875.

In some embodiments, the nucleic acid-based gene-regulating system comprises at least two siRNA or shRNA molecules, wherein at least one siRNA or shRNA molecule is a CBLB-targeting siRNA or shRNA molecule and at least one siRNA or shRNA molecule is a NFKBIA-targeting siRNA or shRNA molecule. In some embodiments, the at least one CBLB-targeting nucleic acid molecule is an siRNA or an shRNA molecule and at least one NFKBIA-targeting nucleic acid molecule is an siRNA or shRNA molecule. In some embodiments, the at least one CBLB-targeting siRNA or an shRNA molecule binds to a target RNA sequence that is at least 95%, 96%, 97%, 98%, or 99% identical to an RNA sequence encoded by the CBLB gene (SEQ ID NO: 7) or the Cblb gene (SEQ ID NO: 8) and the at least one NFKBIA-targeting siRNA or shRNA molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one CBLB-targeting siRNA or an shRNA molecule binds to a target RNA sequence that is 100% identical to an RNA sequence encoded by the CBLB gene (SEQ ID NO: 7) or the Cblb gene (SEQ ID NO: 8) and the at least one NFKBIA-targeting siRNA or shRNA molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one CBLB-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 17 or Table 18 and the at least one NFKBIA-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one CBLB-targeting siRNA or shRNA molecule binds to a target RNA sequence that is 100% identical to a RNA sequence encoded by a DNA sequence defined by a set of genomic coordinates shown in Table 17 or Table 18 and the at least one NFKBIA-targeting siRNA or shRNA molecule binds to a target RNA sequence that is 100% identical to a RNA sequence encoded by a DNA sequence defined by a set of genomic coordinates shown in Table 21 or Table 22.

In some embodiments, the at least one CBLB-targeting siRNA or shRNA molecule binds to a target RNA sequence that is at least 95%, 96%, 97%, 98%, or 99% identical to an RNA sequence encoded by one of SEQ ID NOs: 798-823 and the at least one NFKBIA-targeting siRNA or shRNA molecule binds to a target RNA sequence that is at least 95%, 96%, 97%, 98%, or 99% identical to an RNA sequence encoded by one of SEQ ID NOs: 845-875. In some embodiments, the at least one CBLB-targeting siRNA or shRNA molecule binds to a target RNA sequence that is 100% identical to an RNA sequence encoded by one of SEQ ID NOs: 798-823 and the at least one NFKBIA-targeting siRNA or shRNA molecule binds to a target RNA sequence that is 100% identical to an RNA sequence encoded by one of SEQ ID NOs: 845-875.

In some embodiments, the nucleic acid-based gene-regulating system comprises at least two nucleic acid molecules (e.g., an siRNA, an shRNA, an RNA aptamer, or a morpholino), wherein at least one nucleic acid molecule is a RC3H1-targeting nucleic acid molecule and at least one nucleic acid molecule is a NFKBIA-targeting nucleic acid molecule. In some embodiments, the at least one RC3H1-targeting nucleic acid molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10) and the at least one NFKBIA-targeting nucleic acid molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one RC3H1-targeting nucleic acid molecule binds to a target RNA sequence that is 100% identical to an RNA sequence encoded by the RC3H1 gene (SEQ ID NO: 9) or the Rc3h1 gene (SEQ ID NO: 10) and the at least one NFKBIA-targeting nucleic acid molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one RC3H1-targeting nucleic acid molecule binds to a target RNA sequence that is at least 95%, 96%, 97%, 98%, or 99% identical to a RNA sequence encoded by a DNA sequence defined by a set of genomic coordinates shown in Table 19 or Table 20 and the at least one NFKBIA-targeting nucleic acid molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one RC3H1-targeting nucleic acid molecule binds to a target RNA sequence that is 100% identical to a RNA sequence encoded by a DNA sequence defined by a set of genomic coordinates shown in Table 19 or Table 20 and the at least one NFKBIA-targeting nucleic acid molecule binds to a target RNA sequence that is 100% identical to a RNA sequence encoded by a DNA sequence defined by a set of genomic coordinates shown in Table 21 or Table 22.

In some embodiments, the at least one RC3H1-targeting nucleic acid molecule binds to a target RNA sequence that is at least 95%, 96%, 97%, 98%, or 99% identical to an RNA sequence encoded by one of SEQ ID NOs: 824-844 and the at least one NFKBIA-targeting nucleic acid molecule binds to a target RNA sequence that is at least 95%, 96%, 97%, 98%, or 99% identical to an RNA sequence encoded by one of SEQ ID NOs: 845-875. In some embodiments, the at least one RC3H1-targeting nucleic acid molecule binds to a target RNA sequence that is 100% identical to an RNA sequence encoded by one of SEQ ID NOs: 824-844 and the at least one NFKBIA-targeting nucleic acid molecule binds to a target RNA sequence that is 100% identical to an RNA sequence encoded by one of SEQ ID NOs: 845-875.

In some embodiments, the nucleic acid-based gene-regulating system comprises at least two siRNA or shRNA molecules, wherein at least one siRNA or shRNA molecule is a RC3H1-targeting siRNA or shRNA molecule and at least one siRNA or shRNA molecule is a NFKBIA-targeting siRNA or shRNA molecule. In some embodiments, the at least one RC3H1-targeting nucleic acid molecule is an siRNA or an shRNA molecule and at least one NFKBIA-targeting nucleic acid molecule is an siRNA or shRNA molecule. In some embodiments, the at least one RC3H1-targeting siRNA or an shRNA molecule binds to a target RNA sequence that is at least 95%, 96%, 97%, 98%, or 99% identical to an RNA sequence encoded by the RC3H1 gene (SEQ ID NO: 9) or the Rc3h1 gene (SEQ ID NO: 10) and the at least one NFKBIA-targeting siRNA or shRNA molecule binds to a target RNA sequence that is at least 95%, 96%, 97%, 98%, or 99% identical to an RNA sequence encoded by the NFKBIA gene (SEQ ID NO: 11) or the Nfkbia gene (SEQ ID NO: 12). In some embodiments, the at least one RC3H1-targeting siRNA or an shRNA molecule binds to a target RNA sequence that is 100% identical to an RNA sequence encoded by the RC3H1 gene (SEQ ID NO: 9) or the Rc3h1 gene (SEQ ID NO: 10) and the at least one NFKBIA-targeting siRNA or shRNA molecule binds to a target RNA sequence that is 100% identical to an RNA sequence encoded by the NFKBIA gene (SEQ ID NO: 11) or the Nfkbia gene (SEQ ID NO: 12).

In some embodiments, the at least one RC3H1-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 19 or Table 20 and the at least one NFKBIA-targeting siRNA or shRNA molecule binds to a target RNA sequence that is at least 95%, 96%, 97%, 98%, or 99% identical to a RNA sequence encoded by a DNA sequence defined by a set of genomic coordinates shown in Table 21 or Table 22. In some embodiments, the at least one RC3H1-targeting siRNA or shRNA molecule binds to a target RNA sequence that is 100% identical to a RNA sequence encoded by a DNA sequence defined by a set of genomic coordinates shown in Table 19 or Table 20 and the at least one NFKBIA-targeting siRNA or shRNA molecule binds to a target RNA sequence that is 100% identical to a RNA sequence encoded by a DNA sequence defined by a set of genomic coordinates shown in Table 21 or Table 22.

In some embodiments, the at least one RC3H1-targeting siRNA or shRNA molecule binds to a target RNA sequence that is at least 95%, 96%, 97%, 98%, or 99% identical to an RNA sequence encoded by one of SEQ ID NOs: 824-844 and the at least one NFKBIA-targeting siRNA or shRNA molecule binds to a target RNA sequence that is at least 95%, 96%, 97%, 98%, or 99% identical to an RNA sequence encoded by one of SEQ ID NOs: 845-875. In some embodiments, the at least one RC3H1-targeting siRNA or shRNA molecule binds to a target RNA sequence that is 100% identical to an RNA sequence encoded by one of SEQ ID NOs: 824-844 and the at least one NFKBIA-targeting siRNA or shRNA molecule binds to a target RNA sequence that is 100% identical to an RNA sequence encoded by one of SEQ ID NOs: 845-875.

### B. Protein-Based Gene-Regulating Systems

In some embodiments, the present disclosure provides protein gene-regulating systems comprising one, two or more proteins capable of reducing the expression and/or function of at least one, two or more endogenous genes selected from ANKRD11, BCL2L11, BCL3, BCOR, CALM2, CBLB, CHIC2, CTLA4, DHODH, E2F8, EGR2, FLI1, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.) In some embodiments, the present disclosure provides protein gene-regulating systems comprising one, two or more proteins capable of reducing the expression and/or function of at least one, two or more endogenous genes selected from SOCS1, PTPN2, ZC3H12A, CBLB, RC3H1 and NFKBIA. In some embodiments, the present disclosure provides modified TILs manufactured by the methods described herein comprising such gene-regulating systems. In some embodiments, a protein-based gene-regulating system is a system comprising one or more proteins capable of regulating the expression of an endogenous target gene in a sequence specific manner without the requirement for a nucleic acid guide molecule. In some embodiments, the protein-based gene-regulating system comprises a protein comprising one or more zinc-finger binding domains and an enzymatic domain. In some embodiments, the protein-based gene-regulating system comprises a protein comprising a Transcription activator-like effector nuclease (TALEN) domain and an enzymatic domain. Such embodiments are referred to herein as “TALENs”.

### 1. Zinc Finger Systems

In some embodiments, the present disclosure provides zinc finger gene-regulating systems comprising one, two or more zinc finger fusion proteins capable of reducing the expression and/or function of at least one, two or more endogenous genes selected from SOCS1, PTPN2, ZC3H12A, CBLB, RC3H1 and NFKBIA. In some embodiments, the present disclosure provides modified TILs manufactured by the methods described herein comprising such gene-regulating systems. Herein, zinc finger-based systems comprise a fusion protein with two protein domains: a zinc finger DNA binding domain and an enzymatic domain. A “zinc finger DNA binding domain”, “zinc finger protein”, or “ZFP” is a protein, or a domain within a larger protein, that binds DNA in a sequence-specific manner through one or more zinc fingers, which are regions of amino acid sequence within the binding domain whose structure is stabilized through coordination of a zinc ion. The zinc finger domain, by binding to a target DNA sequence, directs the activity of the enzymatic domain to the vicinity of the sequence and, hence, induces modification of the endogenous target gene in the vicinity of the target sequence. A zinc finger domain can be engineered to bind to virtually any desired sequence. Accordingly, after identifying a target genetic locus containing a target DNA sequence at which cleavage or recombination is desired (e.g., a target locus in a target gene referenced in Tables 2 or 3), one or more zinc finger binding domains can be engineered to bind to one or more target DNA sequences in the target genetic locus. Expression of a fusion protein comprising a zinc finger binding domain and an enzymatic domain in a cell, effects modification in the target genetic locus.

In some embodiments, a zinc finger binding domain comprises one or more zinc fingers. Miller et al. (1985) EMBO J. 4:1609-1614; Rhodes (1993) Scientific American February: 56-65; U.S. Pat. No. 6,453,242. Typically, a single zinc finger domain is about 30 amino acids in length. An individual zinc finger binds to a three-nucleotide (i.e., triplet) sequence (or a four-nucleotide sequence which can overlap, by one nucleotide, with the four-nucleotide binding site of an adjacent zinc finger). Therefore, the length of a sequence to which a zinc finger binding domain is engineered to bind (e.g., a target sequence) will determine the number of zinc fingers in an engineered zinc finger binding domain. For example, for ZFPs in which the finger motifs do not bind to overlapping subsites, a six-nucleotide target sequence is bound by a two-finger binding domain; a nine-nucleotide target sequence is bound by a three-finger binding domain, etc. Binding sites for individual zinc fingers (i.e., subsites) in a target site need not be contiguous, but can be separated by one or several nucleotides, depending on the length and nature of the amino acids sequences between the zinc fingers (i.e., the inter-finger linkers) in a multi-finger binding domain. In some embodiments, the DNA-binding domains of individual ZFPs comprise between three and six individual zinc finger repeats and can each recognize between 9 and 18 base pairs.

Zinc finger binding domains can be engineered to bind to a sequence of choice. See, for example, Beerli et al. (2002) Nature Biotechnol. 20:135-141; Pabo et al. (2001) Ann. Rev. Biochem. 70:313-340; Isalan et al. (2001) Nature Biotechnol. 19:656-660; Segal et al. (2001) Curr. Opin. Biotechnol. 12:632-637; Choo et al. (2000) Curr. Opin. Struct. Biol. 10:411-416. An engineered zinc finger binding domain can have a novel binding specificity, compared to a naturally-occurring zinc finger protein. Engineering methods include, but are not limited to, rational design and various types of selection.

Selection of a target DNA sequence for binding by a zinc finger domain can be accomplished, for example, according to the methods disclosed in U.S. Pat. No. 6,453,242. It will be clear to those skilled in the art that simple visual inspection of a nucleotide sequence can also be used for selection of a target DNA sequence. Accordingly, any means for target DNA sequence selection can be used in the methods described herein. A target site generally has a length of at least 9 nucleotides and, accordingly, is bound by a zinc finger binding domain comprising at least three zinc fingers. However, binding of, for example, a 4-finger binding domain to a 12-nucleotide target site, a 5-finger binding domain to a 15-nucleotide target site or a 6-finger binding domain to an 18-nucleotide target site, is also possible. As will be apparent, binding of larger binding domains (e.g., 7-, 8-, 9-finger and more) to longer target sites is also possible.

In some embodiments, the protein-based gene-regulating system comprises at least one zinc finger fusion protein (ZFP) that comprises a SOCS1-targeting zinc finger binding domain. In some embodiments, the at least one SOCS1-targeting zinc finger binding domain binds to a target DNA sequence that is at least 95%, 96%, 97%, 98%, or 99% identical to a target DNA sequence in the SOCS1 gene (SEQ ID NO: 1) or the Socs1 gene (SEQ ID NO: 2). In some embodiments, the at least one SOCS1-targeting zinc finger binding domain binds to a target DNA sequence that is 100% identical to a target DNA sequence in the SOCS1 gene (SEQ ID NO: 1) or the Socs1 gene (SEQ ID NO: 2).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 4 or Table 5. In some embodiments, the at least one SOCS1-targeting zinc finger binding domain binds to a target DNA sequence that is 100% identical to a DNA sequence defined by a set of genomic coordinates shown in Table 4 or Table 5. In some embodiments, the at least one SOCS1-targeting zinc finger binding domain binds to a target DNA sequence that is at least 90%, 95%, 96%, 97%, 98%, or 99% identical to one of SEQ ID NOs: 23-200. In some embodiments, the at least one SOCS1-targeting zinc finger binding domain binds to a target DNA sequence that is 100% identical to one of SEQ ID NOs: 23-200.

In some embodiments, the protein-based gene-regulating system comprises at least one zinc finger fusion protein (ZFP) that comprises a PTPN2-targeting zinc finger binding domain. In some embodiments, the at least one PTPN2-targeting zinc finger binding domain binds to a target DNA sequence that is at least 95%, 96%, 97%, 98%, or 99% identical to a target DNA sequence in the PTPN2 gene (SEQ ID NO: 3) or the Ptpn2 gene (SEQ ID NO: 4). In some embodiments, the at least one PTPN2-targeting zinc finger binding domain binds to a target DNA sequence that is 100% identical to a target DNA sequence in the PTPN2 gene (SEQ ID NO: 3) or the Ptpn2 gene (SEQ ID NO: 4).

In some embodiments, the at least one PTPN2-targeting zinc finger binding domain binds to a target DNA sequence that is at least 95%, 96%, 97%, 98%, or 99% identical to a DNA sequence defined by a set of genomic coordinates shown in Table 9 or Table 10. In some embodiments, the at least one PTPN2-targeting zinc finger binding domain binds to a target DNA sequence that is 100% identical to a DNA sequence defined by a set of genomic coordinates shown in Table 9 or Table 10. In some embodiments, the at least one PTPN2-targeting zinc finger binding domain binds to a target DNA sequence that is at least 95%, 96%, 97%, 98%, or 99% identical to one of SEQ ID NOs: 201-327. In some embodiments, the at least one PTPN2-targeting zinc finger binding domain binds to a target DNA sequence that is 100% identical to one of SEQ ID NOs: 201-327.

In some embodiments, the protein-based gene-regulating system comprises at least one zinc finger fusion protein (ZFP) that comprises a ZC3H12A-targeting zinc finger binding domain. In some embodiments, the at least one ZC3H12A-targeting zinc finger binding domain binds to a target DNA sequence that is at least 95%, 96%, 97%, 98%, or 99% identical to a target DNA sequence in the ZC3H12A gene (SEQ ID NO: 5) or the Zc3h12a gene (SEQ ID NO: 6). In some embodiments, the at least one ZC3H12A-targeting zinc finger binding domain binds to a target DNA sequence that is 100% identical to a target DNA sequence in the ZC3H12A gene (SEQ ID NO: 5) or the Zc3h12a gene (SEQ ID NO: 6).

In some embodiments, the at least one ZC3H12A-targeting zinc finger binding domain binds to a target DNA sequence that is at least 95%, 96%, 97%, 98%, or 99% identical to a DNA sequence defined by a set of genomic coordinates shown in Table 11 or Table 12. In some embodiments, the at least one ZC3H12A-targeting zinc finger binding domain binds to a target DNA sequence that is 100% identical to a DNA sequence defined by a set of genomic coordinates shown in Table 11 or Table 12. In some embodiments, the at least one ZC3H12A-targeting zinc finger binding domain binds to a target DNA sequence that is at least 95%, 96%, 97%, 98%, or 99% identical to one of SEQ ID NOs: 331-797. In some embodiments, the at least one ZC3H12A-targeting zinc finger binding domain binds to a target DNA sequence that is 100% identical to one of SEQ ID NOs: 331-797.

In some embodiments, the protein-based gene-regulating system comprises at least one TALEN fusion protein that comprises a CBLB-targeting zinc finger binding domain. In some embodiments, the at least one CBLB-targeting zinc finger binding domain binds to a target DNA sequence that is at least 95%, 96%, 97%, 98%, or 99% identical to a target DNA sequence in the CBLB gene (SEQ ID NO: 7) or the Cblb gene (SEQ ID NO: 8). In some embodiments, the at least one CBLB-targeting zinc finger binding domain binds to a target DNA sequence that is 100% identical to a target DNA sequence in the CBLB gene (SEQ ID NO: 7) or the Cblb gene (SEQ ID NO: 8).

In some embodiments, the at least one CBLB-targeting zinc finger binding domain binds to a target DNA sequence that is at least 95%, 96%, 97%, 98%, or 99% identical to a DNA sequence defined by a set of genomic coordinates shown in Table 17 or Table 18. In some embodiments, the at least one CBLB-targeting zinc finger binding domain binds to a target DNA sequence that is 100% identical to a DNA sequence defined by a set of genomic coordinates shown in Table 17 or Table 18. In some embodiments, the at least one CBLB-targeting zinc finger binding domain binds to a target DNA sequence that is at least 95%, 96%, 97%, 98%, or 99% identical to one of SEQ ID NOs: 798-823. In some embodiments, the at least one CBLB-targeting zinc finger binding domain binds to a target DNA sequence that is 100% identical to one of SEQ ID NOs: 798-823.

In some embodiments, the protein-based gene-regulating system comprises at least one TALEN fusion protein that comprises a RC3H1-targeting zinc finger binding domain. In some embodiments, the at least one RC3H1-targeting zinc finger binding domain binds to a target DNA sequence that is at least 95%, 96%, 97%, 98%, or 99% identical to a target DNA sequence in the RC3H1 gene (SEQ ID NO: 9) or the Rc3h1 gene (SEQ ID NO: 10). In some embodiments, the at least one RC3H1-targeting zinc finger binding domain binds to a target DNA sequence that is 100% identical to a target DNA sequence in the RC3H1 gene (SEQ ID NO: 9) or the Rc3h1 gene (SEQ ID NO: 10).

In some embodiments, the at least one RC3H1-targeting zinc finger binding domain binds to a target DNA sequence that is at least 95%, 96%, 97%, 98%, or 99% identical to a DNA sequence defined by a set of genomic coordinates shown in Table 19 or Table 20. In some embodiments, the at least one RC3H1-targeting zinc finger binding domain binds to a target DNA sequence that is 100% identical to a DNA sequence defined by a set of genomic coordinates shown in Table 19 or Table 20. In some embodiments, the at least one RC3H1-targeting zinc finger binding domain binds to a target DNA sequence that is at least 95%, 96%, 97%, 98%, or 99% identical to one of SEQ ID NOs: 824-844. In some embodiments, the at least one RC3H1-targeting zinc finger binding domain binds to a target DNA sequence that is 100% identical to one of SEQ ID NOs: 824-844.

In some embodiments, the protein-based gene-regulating system comprises at least one TALEN fusion protein that comprises a NFKBIA-targeting zinc finger binding domain. In some embodiments, the at least one NFKBIA-targeting zinc finger binding domain binds to a target DNA sequence that is at least 95%, 96%, 97%, 98%, or 99% identical to a target DNA sequence in the NFKBIA gene (SEQ ID NO: 11) or the Nfkbia gene (SEQ ID NO: 12). In some embodiments, the at least one NFKBIA-targeting zinc finger binding domain binds to a target DNA sequence that is 100% identical to a target DNA sequence in the NFKBIA gene (SEQ ID NO: 11) or the Nfkbia gene (SEQ ID NO: 12).

In some embodiments, the at least one NFKBIA-targeting zinc finger binding domain binds to a target DNA sequence that is at least 95%, 96%, 97%, 98%, or 99% identical to a DNA sequence defined by a set of genomic coordinates shown in Table 21 or Table 22. In some embodiments, the at least one NFKBIA-targeting zinc finger binding domain binds to a target DNA sequence that is 100% identical to a DNA sequence defined by a set of genomic coordinates shown in Table 21 or Table 22. In some embodiments, the at least one NFKBIA-targeting zinc finger binding domain binds to a target DNA sequence that is at least 95%, 96%, 97%, 98%, or 99% identical to one of SEQ ID NOs: 845-875. In some embodiments, the at least one NFKBIA-targeting zinc finger binding domain binds to a target DNA sequence that is 100% identical to one of SEQ ID NOs: 845-875.

In some embodiments, the at least one SOCS1-, PTPN2-, ZC3H12A-, CBLB-, RC3H1- or NFKBIA-targeting ZFP is obtained from commercial suppliers such as Sigma Aldrich, Dharmacon, ThermoFisher, and the like. For example, in some embodiments, the at least one SOCS1, PTPN2, or ZC3H12A ZFP is one shown in Table 25.

TABLE 25
Exemplary SOCS1, PTPN2, and ZC3H12A Zinc Finger Systems

| Target Gene | Zinc Finger System |
|---|---|
| SOCS1 | CompoZr ® Knockout ZFN plasmid Human SOCS1 (NM_003745) |
| | (SigmaAldrich# CKOZFND20320) |
| | CompoZr ® Knockout ZFN plasmid Mouse Socs1 (NM_009896.2) |
| | (SigmaAldrich# CKOZFND41801) |
| PTPN2 | CompoZr ® Knockout ZFN human plasmid PTPN1 (NM_002827) |
| | (SigmaAldrich# CKOZFND2121) |
| | CompoZr ® Knockout ZFN murine plasmid Ptpn1 (NM_011201.3) |
| | (SigmaAldrich# CKOZFND39626) |
| ZC3H12A | CompoZr ® Knockout ZFN Kit, ZFN plasmid Human ZC3H12A |
| | (NM_025079) (SigmaAldrich# CKOZFND23094) |
| | CompoZr ® Knockout ZFN Kit, ZFN plasmid mouse Zc3h12a |
| | (NM_153159.2) (SigmaAldrich# CKOZFND44851) |

In some embodiments, the protein-based gene-regulating system comprises at least two ZFPs, wherein at least one ZFP comprises a SOCS1-targeting zinc finger binding domain and at least one ZFP comprises a PTPN2-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one PTPN2-targeting zinc finger binding domain binds to a target DNA sequence that is at least 95%, 96%, 97%, 98%, or 99% identical to a DNA sequence the PTPN2 gene (SEQ ID NO: 3) or the Ptpn2 gene (SEQ ID NO: 4). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 4 or Table 5 and the at least one PTPN2-targeting zinc finger binding domain binds to a target DNA sequence that is at least 95%, 96%, 97%, 98%, or 99% identical to a DNA sequence defined by a set of genomic coordinates shown in Table 9 or Table 10. In some embodiments, the at least one SOCS1-targeting zinc finger binding domain binds to a target DNA sequence that is 100% identical to a DNA sequence defined by a set of genomic coordinates shown in Table 4 or Table 5 and the at least one PTPN2-targeting zinc finger binding domain binds to a target DNA sequence that is 100% identical to a DNA sequence defined by a set of genomic coordinates shown in Table 9 or Table 10.

In some embodiments, the at least one SOCS1-targeting zinc finger binding domain binds to a target DNA sequence that is at least 95%, 96%, 97%, 98%, or 99% identical to one of SEQ ID NOs: 23-200 or 56-187 and the at least one PTPN2-targeting zinc finger binding domain binds to a target DNA sequence that is at least 95%, 96%, 97%, 98%, or 99% identical to one of SEQ ID NOs: 201-327 or 201-314. In some embodiments, the at least one SOCS1-targeting zinc finger binding domain binds to a target DNA sequence that is 100% identical to one of SEQ ID NOs: 23-200 or 56-187 and the at least one PTPN2-targeting zinc finger binding domain binds to a target DNA sequence that is 100% identical to one of SEQ ID NOs: 201-327 or 201-314.

In some embodiments, the protein-based gene-regulating system comprises at least two ZFPs, wherein at least one ZFP comprises a SOCS1-targeting zinc finger binding domain and at least one ZFP comprises a ZC3H12A-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 4 or Table 5 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 11 or Table 12. In some embodiments, the at least one SOCS1-targeting zinc finger binding domain binds to a target DNA sequence that is 100% identical to a DNA sequence defined by a set of genomic coordinates shown in Table 4 or Table 5 and the at least one ZC3H12A-targeting zinc finger binding domain binds to a target DNA sequence that is 100% identical to a DNA sequence defined by a set of genomic coordinates shown in Table 11 or Table 12.

In some embodiments, the at least one SOCS1-targeting zinc finger binding domain binds to a target DNA sequence that is at least 95%, 96%, 97%, 98%, or 99% identical to one of SEQ ID NOs: 23-200 or 56-187 and the at least one ZC3H12A-targeting zinc finger binding domain binds to a target DNA sequence that is at least 95%, 96%, 97%, 98%, or 99% identical to one of SEQ ID NOs: 331-797 or 338-789. In some embodiments, the at least one SOCS1-targeting zinc finger binding domain binds to a target DNA sequence that is 100% identical to one of SEQ ID NOs: 23-200 or 56-187 and the at least one ZC3H12A-targeting zinc finger binding domain binds to a target DNA sequence that is 100% identical to one of SEQ ID NOs: 331-797 or 338-789.

In some embodiments, the protein-based gene-regulating system comprises at least two ZFPs, wherein at least one ZFP comprises a PTPN2-targeting zinc finger binding domain and at least one ZFP comprises a ZC3H12A-targeting zinc finger binding domain. In some embodiments, the at least one PTPN2-targeting zinc finger binding domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one PTPN2-targeting zinc finger binding domain binds to a target DNA sequence that is at least 95%, 96%, 97%, 98%, or 99% identical to a DNA sequence defined by a set of genomic coordinates shown in Table 9 or Table 10 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 11 or Table 12. In some embodiments, the at least one PTPN2-targeting zinc finger binding domain binds to a target DNA sequence that is 100% identical to a DNA sequence defined by a set of genomic coordinates shown in Table 9 or Table 10 and the at least one ZC3H12A-targeting zinc finger binding domain binds to a target DNA sequence that is 100% identical to a DNA sequence defined by a set of genomic coordinates shown in Table 11 or Table 12.

In some embodiments, the at least one PTPN2-targeting zinc finger binding domain binds to a target DNA sequence that is at least 95%, 96%, 97%, 98%, or 99% identical to one of SEQ ID NOs: 201-327 or 201-314 and the at least one ZC3H12A-targeting zinc finger binding domain binds to a target DNA sequence that is at least 95%, 96%, 97%, 98%, or 99% identical to one of SEQ ID NOs: 331-797 or 338-789. In some embodiments, the at least one PTPN2-targeting zinc finger binding domain binds to a target DNA sequence that is 100% identical to one of SEQ ID NOs: 201-327 or 201-314 and the at least one ZC3H12A-targeting zinc finger binding domain binds to a target DNA sequence that is 100% identical to one of SEQ ID NOs: 331-797 or 338-789.

In some embodiments, the protein-based gene-regulating system comprises at least two ZFPs, wherein at least one ZFP comprises a CBLB-targeting zinc finger binding domain and at least one ZFP comprises a PTPN2-targeting zinc finger binding domain. In some embodiments, the at least one CBLB-targeting zinc finger binding domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one PTPN2-targeting zinc finger binding domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one CBLB-targeting zinc finger binding domain binds to a target DNA sequence that is 100% identical to a DNA sequence in the CBLB gene (SEQ ID NO: 7) or the Cblb gene (SEQ ID NO: 8) and the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4).

In some embodiments, the at least one CBLB-targeting zinc finger binding domain binds to a target DNA sequence that is at least 95%, 96%, 97%, 98%, or 99% identical to a DNA sequence defined by a set of genomic coordinates shown in Table 17 or Table 18 and the at least one PTPN2-targeting zinc finger binding domain binds to a target DNA sequence that is at least 95%, 96%, 97%, 98%, or 99% identical to a DNA sequence defined by a set of genomic coordinates shown in Table 9 or Table 10. In some embodiments, the at least one CBLB-targeting zinc finger binding domain binds to a target DNA sequence that is 100% identical to a DNA sequence defined by a set of genomic coordinates shown in Table 17 or Table 18 and the at least one PTPN2-targeting zinc finger binding domain binds to a target DNA sequence that is 100% identical to a DNA sequence defined by a set of genomic coordinates shown in Table 9 or Table 10.

In some embodiments, the at least one CBLB-targeting zinc finger binding domain binds to a target DNA sequence that is at least 95%, 96%, 97%, 98%, or 99% identical to one of SEQ ID NOs: 798-823 or 798-808 and the at least one PTPN2-targeting zinc finger binding domain binds to a target DNA sequence that is at least 95%, 96%, 97%, 98%, or 99% identical to one of SEQ ID NOs: 201-327 or 201-314. In some embodiments, the at least one CBLB-targeting zinc finger binding domain binds to a target DNA sequence that is 100% identical to one of SEQ ID NOs: 798-823 or 798-808 and the at least one PTPN2-targeting zinc finger binding domain binds to a target DNA sequence that is 100% identical to one of SEQ ID NOs: 201-327 or 201-314.

In some embodiments, the protein-based gene-regulating system comprises at least two ZFPs, wherein at least one ZFP comprises a CBLB-targeting zinc finger binding domain and at least one ZFP comprises a ZC3H12A-targeting zinc finger binding domain. In some embodiments, the at least one CBLB-targeting zinc finger binding domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one CBLB-targeting zinc finger binding domain binds to a target DNA sequence that is 100% identical to a DNA sequence in the CBLB gene (SEQ ID NO: 7) or the Cblb gene (SEQ ID NO: 8) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one CBLB-targeting zinc finger binding domain binds to a target DNA sequence that is at least 95%, 96%, 97%, 98%, or 99% identical to a DNA sequence defined by a set of genomic coordinates shown in Table 17 or Table 18 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 11 or Table 12. In some embodiments, the at least one CBLB-targeting zinc finger binding domain binds to a target DNA sequence that is 100% identical to a DNA sequence defined by a set of genomic coordinates shown in Table 17 or Table 18 and the at least one ZC3H12A-targeting zinc finger binding domain binds to a target DNA sequence that is 100% identical to a DNA sequence defined by a set of genomic coordinates shown in Table 11 or Table 12.

In some embodiments, the at least one CBLB-targeting zinc finger binding domain binds to a target DNA sequence that is at least 95%, 96%, 97%, 98%, or 99% identical to one of SEQ ID NOs: 798-823 or 798-808 and the at least one ZC3H12A-targeting zinc finger binding domain binds to a target DNA sequence that is at least 95%, 96%, 97%, 98%, or 99% identical to one of SEQ ID NOs: 331-797 or 338-789. In some embodiments, the at least one CBLB-targeting zinc finger binding domain binds to a target DNA sequence that is 100% identical to one of SEQ ID NOs: 798-823 or 798-808 and the at least one ZC3H12A-targeting zinc finger binding domain binds to a target DNA sequence that is 100% identical to one of SEQ ID NOs: 331-797 or 338-789.

In some embodiments, the protein-based gene-regulating system comprises at least two ZFPs, wherein at least one ZFP comprises a SOCS1-targeting zinc finger binding domain and at least one ZFP comprises a CBLB-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one CBLB-targeting zinc finger binding domain binds to a target DNA sequence that is at least 95%, 96%, 97%, 98%, or 99% identical to a DNA sequence in the CBLB gene (SEQ ID NO: 7) or the Cblb gene (SEQ ID NO: 8). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one CBLB-targeting zinc finger binding domain binds to a target DNA sequence that is 100% identical to a DNA sequence in the CBLB gene (SEQ ID NO: 7) or the Cblb gene (SEQ ID NO: 8).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 4 or Table 5 and the at least one CBLB-targeting zinc finger binding domain binds to a target DNA sequence that is at least 95%, 96%, 97%, 98%, or 99% identical to a DNA sequence defined by a set of genomic coordinates shown in Table 17 or Table 18. In some embodiments, the at least one SOCS1-targeting zinc finger binding domain binds to a target DNA sequence that is 100% identical to a DNA sequence defined by a set of genomic coordinates shown in Table 4 or Table 5 and the at least one CBLB-targeting zinc finger binding domain binds to a target DNA sequence that is 100% identical to a DNA sequence defined by a set of genomic coordinates shown in Table 17 or Table 18.

In some embodiments, the at least one SOCS1-targeting zinc finger binding domain binds to a target DNA sequence that is at least 95%, 96%, 97%, 98%, or 99% identical to one of SEQ ID NOs: 23-200 or 56-187 and the at least one CBLB-targeting zinc finger binding domain binds to a target DNA sequence that is at least 95%, 96%, 97%, 98%, or 99% identical to one of SEQ ID NOs: 798-823 or 798-808. In some embodiments, the at least one SOCS1-targeting zinc finger binding domain binds to a target DNA sequence that is 100% identical to one of SEQ ID NOs: 23-200 or 56-187 and the at least one CBLB-targeting zinc finger binding domain binds to a target DNA sequence that is 100% identical to one of SEQ ID NOs: 798-823 or 798-808.

In some embodiments, the protein-based gene-regulating system comprises at least two ZFPs, wherein at least one ZFP comprises a RC3H1-targeting zinc finger binding domain and at least one ZFP comprises a PTPN2-targeting zinc finger binding domain. In some embodiments, the at least one RC3H1-targeting zinc finger binding domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one PTPN2-targeting zinc finger binding domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one RC3H1-targeting zinc finger binding domain binds to a target DNA sequence that is 100% identical to a DNA sequence in the RC3H1 gene (SEQ ID NO: 9) or the Rc3h1 gene (SEQ ID NO: 10) and the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4).

In some embodiments, the at least one RC3H1-targeting zinc finger binding domain binds to a target DNA sequence that is at least 95%, 96%, 97%, 98%, or 99% identical to a DNA sequence defined by a set of genomic coordinates shown in Table 19 or Table 20 and the at least one PTPN2-targeting zinc finger binding domain binds to a target DNA sequence that is at least 95%, 96%, 97%, 98%, or 99% identical to a DNA sequence defined by a set of genomic coordinates shown in Table 9 or Table 10. In some embodiments, the at least one RC3H1-targeting zinc finger binding domain binds to a target DNA sequence that is 100% identical to a DNA sequence defined by a set of genomic coordinates shown in Table 19 or Table 20 and the at least one PTPN2-targeting zinc finger binding domain binds to a target DNA sequence that is 100% identical to a DNA sequence defined by a set of genomic coordinates shown in Table 9 or Table 10.

In some embodiments, the at least one RC3H1-targeting zinc finger binding domain binds to a target DNA sequence that is at least 95%, 96%, 97%, 98%, or 99% identical to one of SEQ ID NOs: 824-844 or 824-836 and the at least one PTPN2-targeting zinc finger binding domain binds to a target DNA sequence that is at least 95%, 96%, 97%, 98%, or 99% identical to one of SEQ ID NOs: 201-327 or 201-314. In some embodiments, the at least one RC3H1-targeting zinc finger binding domain binds to a target DNA sequence that is 100% identical to one of SEQ ID NOs: 824-844 or 824-836 and the at least one PTPN2-targeting zinc finger binding domain binds to a target DNA sequence that is 100% identical to one of SEQ ID NOs: 201-327 or 201-314.

In some embodiments, the protein-based gene-regulating system comprises at least two ZFPs, wherein at least one ZFP comprises a RC3H1-targeting zinc finger binding domain and at least one ZFP comprises a ZC3H12A-targeting zinc finger binding domain. In some embodiments, the at least one RC3H1-targeting zinc finger binding domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one RC3H1-targeting zinc finger binding domain binds to a target DNA sequence that is 100% identical to a DNA sequence in the RC3H1 gene (SEQ ID NO: 9) or the Rc3h1 gene (SEQ ID NO: 10) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one RC3H1-targeting zinc finger binding domain binds to a target DNA sequence that is at least 95%, 96%, 97%, 98%, or 99% identical to a DNA sequence defined by a set of genomic coordinates shown in Table 19 or Table 20 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 11 or Table 12. In some embodiments, the at least one RC3H1-targeting zinc finger binding domain binds to a target DNA sequence that is 100% identical to a DNA sequence defined by a set of genomic coordinates shown in Table 19 or Table 20 and the at least one ZC3H12A-targeting zinc finger binding domain binds to a target DNA sequence that is 100% identical to a DNA sequence defined by a set of genomic coordinates shown in Table 11 or Table 12.

In some embodiments, the at least one RC3H1-targeting zinc finger binding domain binds to a target DNA sequence that is at least 95%, 96%, 97%, 98%, or 99% identical to one of SEQ ID NOs: 824-844 or 824-836 and the at least one ZC3H12A-targeting zinc finger binding domain binds to a target DNA sequence that is at least 95%, 96%, 97%, 98%, or 99% identical to one of SEQ ID NOs: 331-797 or 338-789. In some embodiments, the at least one RC3H1-targeting zinc finger binding domain binds to a target DNA sequence that is 100% identical to one of SEQ ID NOs: 824-844 or 824-836 and the at least one ZC3H12A-targeting zinc finger binding domain binds to a target DNA sequence that is 100% identical to one of SEQ ID NOs: 331-797 or 338-789.

In some embodiments, the protein-based gene-regulating system comprises at least two ZFPs, wherein at least one ZFP comprises a SOCS1-targeting zinc finger binding domain and at least one ZFP comprises a RC3H1-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one RC3H1-targeting zinc finger binding domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one RC3H1-targeting zinc finger binding domain binds to a target DNA sequence that is 100% identical to a DNA sequence in the RC3H1 gene (SEQ ID NO: 9) or the Rc3h1 gene (SEQ ID NO: 10).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 4 or Table 5 and the at least one RC3H1-targeting zinc finger binding domain binds to a target DNA sequence that is at least 95%, 96%, 97%, 98%, or 99% identical to a DNA sequence defined by a set of genomic coordinates shown in Table 19 or Table 20. In some embodiments, the at least one SOCS1-targeting zinc finger binding domain binds to a target DNA sequence that is 100% identical to a DNA sequence defined by a set of genomic coordinates shown in Table 4 or Table 5 and the at least one RC3H1-targeting zinc finger binding domain binds to a target DNA sequence that is 100% identical to a DNA sequence defined by a set of genomic coordinates shown in Table 19 or Table 20.

In some embodiments, the protein-based gene-regulating system comprises at least two ZFPs, wherein at least one ZFP comprises a CBLB-targeting zinc finger binding domain and at least one ZFP comprises a RC3H1-targeting zinc finger binding domain. In some embodiments, the at least one CBLB-targeting zinc finger binding domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one RC3H1-targeting zinc finger binding domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10). In some embodiments, the at least one CBLB-targeting zinc finger binding domain binds to a target DNA sequence that is 100% identical to a DNA sequence in the CBLB gene (SEQ ID NO: 7) or the Cblb gene (SEQ ID NO: 8) and the at least one RC3H1-targeting zinc finger binding domain binds to a target DNA sequence that is 100% identical to a DNA sequence in the RC3H1 gene (SEQ ID NO: 9) or the Rc3h1 gene (SEQ ID NO: 10).

In some embodiments, the at least one CBLB-targeting zinc finger binding domain binds to a target DNA sequence that is at least 95%, 96%, 97%, 98%, or 99% identical to a DNA sequence defined by a set of genomic coordinates shown in Table 17 or Table 18 and the at least one RC3H1-targeting zinc finger binding domain binds to a target DNA sequence that is at least 95%, 96%, 97%, 98%, or 99% identical to a DNA sequence defined by a set of genomic coordinates shown in Table 19 or Table 20. In some embodiments, the at least one CBLB-targeting zinc finger binding domain binds to a target DNA sequence that is 100% identical to a DNA sequence defined by a set of genomic coordinates shown in Table 17 or Table 18 and the at least one RC3H1-targeting zinc finger binding domain binds to a target DNA sequence that is 100% identical to a DNA sequence defined by a set of genomic coordinates shown in Table 19 or Table 20.

In some embodiments, the at least one CBLB-targeting zinc finger binding domain binds to a target DNA sequence that is at least 95%, 96%, 97%, 98%, or 99% identical to one of SEQ ID NOs: 798-823 or 798-808 and the at least one RC3H1-targeting zinc finger binding domain binds to a target DNA sequence that is at least 95%, 96%, 97%, 98%, or 99% identical to one of SEQ ID NOs: 824-844 or 824-836. In some embodiments, the at least one CBLB-targeting zinc finger binding domain binds to a target DNA sequence that is 100% identical to one of SEQ ID NOs: 798-823 or 798-808 and the at least one RC3H1-targeting zinc finger binding domain binds to a target DNA sequence that is 100% identical to one of SEQ ID NOs: 824-844 or 824-836.

In some embodiments, the protein-based gene-regulating system comprises at least two ZFPs, wherein at least one ZFP comprises a NFKBIA-targeting zinc finger binding domain and at least one ZFP comprises a PTPN2-targeting zinc finger binding domain. In some embodiments, the at least one NFKBIA-targeting zinc finger binding domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12) and the at least one PTPN2-targeting zinc finger binding domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one NFKBIA-targeting zinc finger binding domain binds to a target DNA sequence that is 100% identical to a DNA sequence in the NFKBIA gene (SEQ ID NO: 11) or the Nfkbia gene (SEQ ID NO: 12) and the at least one PTPN2-targeting zinc finger binding domain binds to a target DNA sequence that is 100% identical to a DNA sequence in the PTPN2 gene (SEQ ID NO: 3) or the Ptpn2 gene (SEQ ID NO: 4).

In some embodiments, the at least one NFKBIA-targeting zinc finger binding domain binds to a target DNA sequence that is at least 95%, 96%, 97%, 98%, or 99% identical to a DNA sequence defined by a set of genomic coordinates shown in Table 21 or Table 22 and the at least one PTPN2-targeting zinc finger binding domain binds to a target DNA sequence that is at least 95%, 96%, 97%, 98%, or 99% identical to a DNA sequence defined by a set of genomic coordinates shown in Table 9 or Table 10. In some embodiments, the at least one NFKBIA-targeting zinc finger binding domain binds to a target DNA sequence that is 100% identical to a DNA sequence defined by a set of genomic coordinates shown in Table 21 or Table 22 and the at least one PTPN2-targeting zinc finger binding domain binds to a target DNA sequence that is 100% identical to a DNA sequence defined by a set of genomic coordinates shown in Table 9 or Table 10.

In some embodiments, the at least one NFKBIA-targeting zinc finger binding domain binds to a target DNA sequence that is at least 95%, 96%, 97%, 98%, or 99% identical to one of SEQ ID NOs: 845-875 or 845-856 and the at least one PTPN2-targeting zinc finger binding domain binds to a target DNA sequence that is at least 95%, 96%, 97%, 98%, or 99% identical to one of SEQ ID NOs: 201-327 or 201-314. In some embodiments, the at least one NFKBIA-targeting zinc finger binding domain binds to a target DNA sequence that is 100% identical to one of SEQ ID NOs: 845-875 or 845-856 and the at least one PTPN2-targeting zinc finger binding domain binds to a target DNA sequence that is 100% identical to one of SEQ ID NOs: 201-327 or 201-314.

In some embodiments, the protein-based gene-regulating system comprises at least two ZFPs, wherein at least one ZFP comprises a NFKBIA-targeting zinc finger binding domain and at least one ZFP comprises a ZC3H12A-targeting zinc finger binding domain. In some embodiments, the at least one NFKBIA-targeting zinc finger binding domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12) and the at least one ZC3H12A-targeting zinc finger binding domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one NFKBIA-targeting zinc finger binding domain binds to a target DNA sequence that is 100% identical to a DNA sequence in the NFKBIA gene (SEQ ID NO: 11) or the Nfkbia gene (SEQ ID NO: 12) and the at least one ZC3H12A-targeting zinc finger binding domain binds to a target DNA sequence that is 100% identical to a DNA sequence in the ZC3H12A gene (SEQ ID NO: 5) or the Zc3h12a gene (SEQ ID NO: 6).

In some embodiments, the at least one NFKBIA-targeting zinc finger binding domain binds to a target DNA sequence that is at least 95%, 96%, 97%, 98%, or 99% identical to a DNA sequence defined by a set of genomic coordinates shown in Table 21 or Table 22 and the at least one ZC3H12A-targeting zinc finger binding domain binds to a target DNA sequence that is at least 95%, 96%, 97%, 98%, or 99% identical to a DNA sequence defined by a set of genomic coordinates shown in Table 11 or Table 12. In some embodiments, the at least one NFKBIA-targeting zinc finger binding domain binds to a target DNA sequence that is 100% identical to a DNA sequence defined by a set of genomic coordinates shown in Table 21 or Table 22 and the at least one ZC3H12A-targeting zinc finger binding domain binds to a target DNA sequence that is 100% identical to a DNA sequence defined by a set of genomic coordinates shown in Table 11 or Table 12.

In some embodiments, the at least one NFKBIA-targeting zinc finger binding domain binds to a target DNA sequence that is at least 95%, 96%, 97%, 98%, or 99% identical to one of SEQ ID NOs: 845-875 or 845-856 and the at least one ZC3H12A-targeting zinc finger binding domain binds to a target DNA sequence that is at least 95%, 96%, 97%, 98%, or 99% identical to one of SEQ ID NOs: 331-797 or 338-789. In some embodiments, the at least one NFKBIA-targeting zinc finger binding domain binds to a target DNA sequence that is 100% identical to one of SEQ ID NOs: 845-875 or 845-856 and the at least one ZC3H12A-targeting zinc finger binding domain binds to a target DNA sequence that is 100% identical to one of SEQ ID NOs: 331-797 or 338-789.

In some embodiments, the protein-based gene-regulating system comprises at least two ZFPs, wherein at least one ZFP comprises a SOCS1-targeting zinc finger binding domain and at least one ZFP comprises a NFKBIA-targeting zinc finger binding domain. In some embodiments, the at least one SOCS1-targeting zinc finger binding domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one NFKBIA-targeting zinc finger binding domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one SOCS1-targeting zinc finger binding domain binds to a target DNA sequence that is 100% identical to a DNA sequence in the SOCS1 gene (SEQ ID NO: 1) or the Socs1 gene (SEQ ID NO: 2) and the at least one NFKBIA-targeting zinc finger binding domain binds to a target DNA sequence that is 100% identical to a DNA sequence in the NFKBIA gene (SEQ ID NO: 11) or the Nfkbia gene (SEQ ID NO: 12).

In some embodiments, the at least one SOCS1-targeting zinc finger binding domain binds to a target DNA sequence that is at least 95%, 96%, 97%, 98%, or 99% identical to a DNA sequence defined by a set of genomic coordinates shown in Table 4 or Table 5 and the at least one NFKBIA-targeting zinc finger binding domain binds to a target DNA sequence that is at least 95%, 96%, 97%, 98%, or 99% identical to a DNA sequence defined by a set of genomic coordinates shown in Table 21 or Table 22. In some embodiments, the at least one SOCS1-targeting zinc finger binding domain binds to a target DNA sequence that is 100% identical to a DNA sequence defined by a set of genomic coordinates shown in Table 4 or Table 5 and the at least one NFKBIA-targeting zinc finger binding domain binds to a target DNA sequence that is 100% identical to a DNA sequence defined by a set of genomic coordinates shown in Table 21 or Table 22.

In some embodiments, the at least one SOCS1-targeting zinc finger binding domain binds to a target DNA sequence that is at least 95%, 96%, 97%, 98%, or 99% identical to one of SEQ ID NOs: 23-200 or 56-187 and the at least one NFKBIA-targeting zinc finger binding domain binds to a target DNA sequence that is at least 95%, 96%, 97%, 98%, or 99% identical to one of SEQ ID NOs: 845-875 or 845-856. In some embodiments, the at least one SOCS1-targeting zinc finger binding domain binds to a target DNA sequence that is 100% identical to one of SEQ ID NOs: 23-200 or 56-187 and the at least one NFKBIA-targeting zinc finger binding domain binds to a target DNA sequence that is 100% identical to one of SEQ ID NOs: 845-875 or 845-856.

In some embodiments, the protein-based gene-regulating system comprises at least two ZFPs, wherein at least one ZFP comprises a CBLB-targeting zinc finger binding domain and at least one ZFP comprises a NFKBIA-targeting zinc finger binding domain. In some embodiments, the at least one CBLB-targeting zinc finger binding domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one NFKBIA-targeting zinc finger binding domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one CBLB-targeting zinc finger binding domain binds to a target DNA sequence that is 100% identical to a DNA sequence in the CBLB gene (SEQ ID NO: 7) or the Cblb gene (SEQ ID NO: 8) and the at least one NFKBIA-targeting zinc finger binding domain binds to a target DNA sequence that is 100% identical to a DNA sequence in the NFKBIA gene (SEQ ID NO: 11) or the Nfkbia gene (SEQ ID NO: 12).

In some embodiments, the at least one CBLB-targeting zinc finger binding domain binds to a target DNA sequence that is at least 95%, 96%, 97%, 98%, or 99% identical to a DNA sequence defined by a set of genomic coordinates shown in Table 17 or Table 18 and the at least one NFKBIA-targeting zinc finger binding domain binds to a target DNA sequence that is at least 95%, 96%, 97%, 98%, or 99% identical to a DNA sequence defined by a set of genomic coordinates shown in Table 21 or Table 22. In some embodiments, the at least one CBLB-targeting zinc finger binding domain binds to a target DNA sequence that is 100% identical to a DNA sequence defined by a set of genomic coordinates shown in Table 17 or Table 18 and the at least one NFKBIA-targeting zinc finger binding domain binds to a target DNA sequence that is 100% identical to a DNA sequence defined by a set of genomic coordinates shown in Table 21 or Table 22.

In some embodiments, the at least one CBLB-targeting zinc finger binding domain binds to a target DNA sequence that is at least 95%, 96%, 97%, 98%, or 99% identical to one of SEQ ID NOs: 798-823 or 798-808 and the at least one NFKBIA-targeting zinc finger binding domain binds to a target DNA sequence that is at least 95%, 96%, 97%, 98%, or 99% identical to one of SEQ ID NOs: 845-875 or 845-856. In some embodiments, the at least one CBLB-targeting zinc finger binding domain binds to a target DNA sequence that is 100% identical to one of SEQ ID NOs: 798-823 or 798-808 and the at least one NFKBIA-targeting zinc finger binding domain binds to a target DNA sequence that is 100% identical to one of SEQ ID NOs: 845-875 or 845-856.

In some embodiments, the protein-based gene-regulating system comprises at least two ZFPs, wherein at least one ZFP comprises a RC3H1-targeting zinc finger binding domain and at least one ZFP comprises a NFKBIA-targeting zinc finger binding domain. In some embodiments, the at least one RC3H1-targeting zinc finger binding domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one NFKBIA-targeting zinc finger binding domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one RC3H1-targeting zinc finger binding domain binds to a target DNA sequence that is 100% identical to a DNA sequence in the RC3H1 gene (SEQ ID NO: 9) or the Rc3h1 gene (SEQ ID NO: 10) and the at least one NFKBIA-targeting zinc finger binding domain binds to a target DNA sequence that is 100% identical to a DNA sequence in the NFKBIA gene (SEQ ID NO: 11) or the Nfkbia gene (SEQ ID NO: 12).

In some embodiments, the at least one RC3H1-targeting zinc finger binding domain binds to a target DNA sequence that is at least 95%, 96%, 97%, 98%, or 99% identical to a DNA sequence defined by a set of genomic coordinates shown in Table 19 or Table 20 and the at least one NFKBIA-targeting zinc finger binding domain binds to a target DNA sequence that is at least 95%, 96%, 97%, 98%, or 99% identical to a DNA sequence defined by a set of genomic coordinates shown in Table 21 or Table 22. In some embodiments, the at least one RC3H1-targeting zinc finger binding domain binds to a target DNA sequence that is 100% identical to a DNA sequence defined by a set of genomic coordinates shown in Table 19 or Table 20 and the at least one NFKBIA-targeting zinc finger binding domain binds to a target DNA sequence that is 100% identical to a DNA sequence defined by a set of genomic coordinates shown in Table 21 or Table 22.

In some embodiments, the at least one RC3H1-targeting zinc finger binding domain binds to a target DNA sequence that is at least 95%, 96%, 97%, 98%, or 99% identical to one of SEQ ID NOs: 824-844 or 824-836 and the at least one NFKBIA-targeting zinc finger binding domain binds to a target DNA sequence that is at least 95%, 96%, 97%, 98%, or 99% identical to one of SEQ ID NOs: 845-875 or 845-856. In some embodiments, the at least one RC3H1-targeting zinc finger binding domain binds to a target DNA sequence that is 100% identical to one of SEQ ID NOs: 824-844 or 824-836 and the at least one NFKBIA-targeting zinc finger binding domain binds to a target DNA sequence that is 100% identical to one of SEQ ID NOs: 845-875 or 845-856.

The enzymatic domain portion of the zinc finger fusion proteins can be obtained from any endo- or exonuclease. Exemplary endonucleases from which an enzymatic domain can be derived include, but are not limited to, restriction endonucleases and homing endonucleases. See, for example, 2002-2003 Catalogue, New England Biolabs, Beverly, Mass.; and Belfort et al. (1997) Nucleic Acids Res. 25:3379-3388. Additional enzymes which cleave DNA are known (e.g., 51 Nuclease; mung bean nuclease; pancreatic DNaseI; micrococcal nuclease; yeast HO endonuclease; see also Linn et al. (eds.) Nucleases, Cold Spring Harbor Laboratory Press, 1993). One or more of these enzymes (or functional fragments thereof) can be used as a source of cleavage domains.

Exemplary restriction endonucleases (restriction enzymes) suitable for use as an enzymatic domain of the ZFPs described herein are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31,978-31,982. Thus, in one embodiment, fusion proteins comprise the enzymatic domain from at least one Type IIS restriction enzyme and one or more zinc finger binding domains.

An exemplary Type IIS restriction enzyme, whose cleavage domain is separable from the binding domain, is FokI. This particular enzyme is active as a dimer. Bitinaite et al. (1998) Proc. Natl. Acad. Sci. USA 95: 10,570-10,575. Thus, for targeted double-stranded DNA cleavage using zinc finger-FokI fusions, two fusion proteins, each comprising a FokI enzymatic domain, can be used to reconstitute a catalytically active cleavage domain. Alternatively, a single polypeptide molecule containing a zinc finger binding domain and two FokI enzymatic domains can also be used. Exemplary ZFPs comprising FokI enzymatic domains are described in U.S. Pat. No. 9,782,437.

### 2. TALEN Systems

In some embodiments, the present disclosure provides TALEN gene-regulating systems comprising one, two or more TALEN fusion proteins capable of reducing the expression and/or function of at least one, two or more endogenous genes selected from SOCS1, PTPN2, ZC3H12A, CBLB, RC3H1 and NFKBIA. In some embodiments, the present disclosure provides modified TILs manufactured by the methods described herein comprising such gene-regulating systems. TALEN-based systems comprise a TALEN fusion protein comprising a TAL effector DNA binding domain and an enzymatic domain. They are made by fusing a TAL effector DNA-binding domain to a DNA cleavage domain (a nuclease which cuts DNA strands). The FokI restriction enzyme described above is an exemplary enzymatic domain suitable for use in TALEN-based gene-regulating systems.

TAL effectors are proteins that are secreted by Xanthomonas bacteria via their type III secretion system when they infect plants. The DNA binding domain contains a repeated, highly conserved, 33-34 amino acid sequence with divergent 12th and 13th amino acids. These two positions, referred to as the Repeat Variable Diresidue (RVD), are highly variable and strongly correlated with specific nucleotide recognition. Therefore, the TAL effector domains can be engineered to bind specific target DNA sequences by selecting a combination of repeat segments containing the appropriate RVDs. The nucleic acid specificity for RVD combinations is as follows: HD targets cytosine, NI targets adenine, NG targets thymine, and NN targets guanine (though, in some embodiments, NN can also bind adenine with lower specificity).

Methods and compositions for assembling the TAL-effector repeats are known in the art. See e.g., Cermak et al, Nucleic Acids Research, 39:12, 2011, e82. Plasmids for constructions of the TAL-effector repeats are commercially available from Addgene.

In some embodiments, the protein-based gene-regulating system comprises at least one TALEN fusion protein that comprises a SOCS1-targeting TAL effector domain. In some embodiments, the at least one SOCS1-targeting TAL effector domain binds to a target DNA sequence that is at least 95%, 96%, 97%, 98%, or 99% identical to a target DNA sequence in the SOCS1 gene (SEQ ID NO: 1) or the Socs1 gene (SEQ ID NO: 2). In some embodiments, the at least one SOCS1-targeting TAL effector domain binds to a target DNA sequence that is 100% identical to a target DNA sequence in the SOCS1 gene (SEQ ID NO: 1) or the Socs1 gene (SEQ ID NO: 2).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 4 or Table 5. In some embodiments, the at least one SOCS1-targeting TAL effector domain binds to a target DNA sequence that is 100% identical to a DNA sequence defined by a set of genomic coordinates shown in Table 4 or Table 5. In some embodiments, the at least one SOCS1-targeting TAL effector domain binds to a target DNA sequence that is at least 90%, 95%, 96%, 97%, 98%, or 99% identical to one of SEQ ID NOs: 23-200 or 56-187. In some embodiments, the at least one SOCS1-targeting TAL effector domain binds to a target DNA sequence that is 100% identical to one of SEQ ID NOs: 23-200 or 56-187.

In some embodiments, the protein-based gene-regulating system comprises at least one TALEN fusion protein that comprises a PTPN2-targeting TAL effector domain. In some embodiments, the at least one PTPN2-targeting TAL effector domain binds to a target DNA sequence that is at least 95%, 96%, 97%, 98%, or 99% identical to a target DNA sequence in the PTPN2 gene (SEQ ID NO: 3) or the Ptpn2 gene (SEQ ID NO: 4). In some embodiments, the at least one PTPN2-targeting TAL effector domain binds to a target DNA sequence that is 100% identical to a target DNA sequence in the PTPN2 gene (SEQ ID NO: 3) or the Ptpn2 gene (SEQ ID NO: 4).

In some embodiments, the at least one PTPN2-targeting TAL effector domain binds to a target DNA sequence that is at least 95%, 96%, 97%, 98%, or 99% identical to a DNA sequence defined by a set of genomic coordinates shown in Table 9 or Table 10. In some embodiments, the at least one PTPN2-targeting TAL effector domain binds to a target DNA sequence that is 100% identical to a DNA sequence defined by a set of genomic coordinates shown in Table 9 or Table 10. In some embodiments, the at least one PTPN2-targeting TAL effector domain binds to a target DNA sequence that is at least 95%, 96%, 97%, 98%, or 99% identical to one of SEQ ID NOs: 201-327 or 201-314. In some embodiments, the at least one PTPN2-targeting TAL effector domain binds to a target DNA sequence that is 100% identical to one of SEQ ID NOs: 201-327 or 201-314.

In some embodiments, the protein-based gene-regulating system comprises at least one TALEN fusion protein that comprises a ZC3H12A-targeting TAL effector domain. In some embodiments, the at least one ZC3H12A-targeting TAL effector domain binds to a target DNA sequence that is at least 95%, 96%, 97%, 98%, or 99% identical to a target DNA sequence in the ZC3H12A gene (SEQ ID NO: 5) or the Zc3h12a gene (SEQ ID NO: 6). In some embodiments, the at least one ZC3H12A-targeting TAL effector domain binds to a target DNA sequence that is 100% identical to a target DNA sequence in the ZC3H12A gene (SEQ ID NO: 5) or the Zc3h12a gene (SEQ ID NO: 6).

In some embodiments, the at least one ZC3H12A-targeting TAL effector domain binds to a target DNA sequence that is at least 95%, 96%, 97%, 98%, or 99% identical to a DNA sequence defined by a set of genomic coordinates shown in Table 11 or Table 12. In some embodiments, the at least one ZC3H12A-targeting TAL effector domain binds to a target DNA sequence that is 100% identical to a DNA sequence defined by a set of genomic coordinates shown in Table 11 or Table 12. In some embodiments, the at least one ZC3H12A-targeting TAL effector domain binds to a target DNA sequence that is at least 95%, 96%, 97%, 98%, or 99% identical to one of SEQ ID NOs: 331-797 or 338-789. In some embodiments, the at least one ZC3H12A-targeting TAL effector domain binds to a target DNA sequence that is 100% identical to one of SEQ ID NOs: 331-797 or 338-789.

In some embodiments, the protein-based gene-regulating system comprises at least one TALEN fusion protein that comprises a CBLB-targeting TAL effector domain. In some embodiments, the at least one CBLB-targeting TAL effector domain binds to a target DNA sequence that is at least 95%, 96%, 97%, 98%, or 99% identical to a target DNA sequence in the CBLB gene (SEQ ID NO: 7) or the Cblb gene (SEQ ID NO: 8). In some embodiments, the at least one CBLB-targeting TAL effector domain binds to a target DNA sequence that is 100% identical to a target DNA sequence in the CBLB gene (SEQ ID NO: 7) or the Cblb gene (SEQ ID NO: 8).

In some embodiments, the at least one CBLB-targeting TAL effector domain binds to a target DNA sequence that is at least 95%, 96%, 97%, 98%, or 99% identical to a DNA sequence defined by a set of genomic coordinates shown in Table 17 or Table 18. In some embodiments, the at least one CBLB-targeting TAL effector domain binds to a target DNA sequence that is 100% identical to a DNA sequence defined by a set of genomic coordinates shown in Table 17 or Table 18. In some embodiments, the at least one CBLB-targeting TAL effector domain binds to a target DNA sequence that is at least 95%, 96%, 97%, 98%, or 99% identical to one of SEQ ID NOs: 798-823 or 798-808. In some embodiments, the at least one CBLB-targeting TAL effector domain binds to a target DNA sequence that is 100% identical to one of SEQ ID NOs: 798-823 or 798-808.

In some embodiments, the protein-based gene-regulating system comprises at least one TALEN fusion protein that comprises a RC3H1-targeting TAL effector domain. In some embodiments, the at least one RC3H1-targeting TAL effector domain binds to a target DNA sequence that is at least 95%, 96%, 97%, 98%, or 99% identical to a target DNA sequence in the RC3H1 gene (SEQ ID NO: 9) or the Rc3h1 gene (SEQ ID NO: 10). In some embodiments, the at least one RC3H1-targeting TAL effector domain binds to a target DNA sequence that is 100% identical to a target DNA sequence in the RC3H1 gene (SEQ ID NO: 9) or the Rc3h1 gene (SEQ ID NO: 10).

In some embodiments, the at least one RC3H1-targeting TAL effector domain binds to a target DNA sequence that is at least 95%, 96%, 97%, 98%, or 99% identical to a DNA sequence defined by a set of genomic coordinates shown in Table 7 or Table 8. In some embodiments, the at least one RC3H1-targeting TAL effector domain binds to a target DNA sequence that is 100% identical to a DNA sequence defined by a set of genomic coordinates shown in Table 19 or Table 20. In some embodiments, the at least one RC3H1-targeting TAL effector domain binds to a target DNA sequence that is at least 95%, 96%, 97%, 98%, or 99% identical to one of SEQ ID NOs: 824-844 or 824-836. In some embodiments, the at least one RC3H1-targeting TAL effector domain binds to a target DNA sequence that is 100% identical to one of SEQ ID NOs: 824-844 or 824-836.

In some embodiments, the protein-based gene-regulating system comprises at least one TALEN fusion protein that comprises a NFKBIA-targeting TAL effector domain. In some embodiments, the at least one NFKBIA-targeting TAL effector domain binds to a target DNA sequence that is at least 95%, 96%, 97%, 98%, or 99% identical to a target DNA sequence in the NFKBIA gene (SEQ ID NO: 11) or the Nfkbia gene (SEQ ID NO: 12). In some embodiments, the at least one NFKBIA-targeting TAL effector domain binds to a target DNA sequence that is 100% identical to a target DNA sequence in the NFKBIA gene (SEQ ID NO: 11) or the Nfkbia gene (SEQ ID NO: 12).

In some embodiments, the at least one NFKBIA-targeting TAL effector domain binds to a target DNA sequence that is at least 95%, 96%, 97%, 98%, or 99% identical to a DNA sequence defined by a set of genomic coordinates shown in Table 21 or Table 22. In some embodiments, the at least one NFKBIA-targeting TAL effector domain binds to a target DNA sequence that is 100% identical to a DNA sequence defined by a set of genomic coordinates shown in Table 21 or Table 22. In some embodiments, the at least one NFKBIA-targeting TAL effector domain binds to a target DNA sequence that is at least 95%, 96%, 97%, 98%, or 99% identical to one of SEQ ID NOs: 845-875 or 845-856. In some embodiments, the at least one NFKBIA-targeting TAL effector domain binds to a target DNA sequence that is 100% identical to one of SEQ ID NOs: 845-875 or 845-856.

In some embodiments, the protein-based gene-regulating system comprises at least two Talen fusion proteins, wherein at least one Talen fusion protein comprises a SOCS1-targeting TAL effector domain and at least one Talen fusion protein comprises a PTPN2-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one PTPN2-targeting TAL effector domain binds to a target DNA sequence that is at least 95%, 96%, 97%, 98%, or 99% identical to a DNA sequence the PTPN2 gene (SEQ ID NO: 3) or the Ptpn2 gene (SEQ ID NO: 4). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 4 or Table 5 and the at least one PTPN2-targeting TAL effector domain binds to a target DNA sequence that is at least 95%, 96%, 97%, 98%, or 99% identical to a DNA sequence defined by a set of genomic coordinates shown in Table 9 or Table 10. In some embodiments, the at least one SOCS1-targeting TAL effector domain binds to a target DNA sequence that is 100% identical to a DNA sequence defined by a set of genomic coordinates shown in Table 4 or Table 5 and the at least one PTPN2-targeting TAL effector domain binds to a target DNA sequence that is 100% identical to a DNA sequence defined by a set of genomic coordinates shown in Table 9 or Table 10.

In some embodiments, the at least one SOCS1-targeting TAL effector domain binds to a target DNA sequence that is at least 95%, 96%, 97%, 98%, or 99% identical to one of SEQ ID NOs: 23-200 or 56-18723-200 or 56-187 and the at least one PTPN2-targeting TAL effector domain binds to a target DNA sequence that is at least 95%, 96%, 97%, 98%, or 99% identical to one of SEQ ID NOs: 201-327 or 201-314. In some embodiments, the at least one SOCS1-targeting TAL effector domain binds to a target DNA sequence that is 100% identical to one of SEQ ID NOs: 23-200 or 56-187 and the at least one PTPN2-targeting TAL effector domain binds to a target DNA sequence that is 100% identical to one of SEQ ID NOs: 201-327 or 201-314.

In some embodiments, the protein-based gene-regulating system comprises at least two Talen fusion proteins, wherein at least one Talen fusion protein comprises a SOCS1-targeting TAL effector domain and at least one Talen fusion protein comprises a ZC3H12A-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 4 or Table 5 and the at least one ZC3H12A-targeting TAL effector domain binds to a target DNA sequence that is at least 95%, 96%, 97%, 98%, or 99% identical to a DNA sequence defined by a set of genomic coordinates shown in Table 11 or Table 12. In some embodiments, the at least one SOCS1-targeting TAL effector domain binds to a target DNA sequence that is 100% identical to a DNA sequence defined by a set of genomic coordinates shown in Table 4 or Table 5 and the at least one ZC3H12A-targeting TAL effector domain binds to a target DNA sequence that is 100% identical to a DNA sequence defined by a set of genomic coordinates shown in Table 11 or Table 12.

In some embodiments, the at least one SOCS1-targeting TAL effector domain binds to a target DNA sequence that is at least 95%, 96%, 97%, 98%, or 99% identical to one of SEQ ID NOs: 23-200 or 56-18723-200 or 56-187 and the at least one ZC3H12A-targeting TAL effector domain binds to a target DNA sequence that is at least 95%, 96%, 97%, 98%, or 99% identical to one of SEQ ID NOs: 331-797 or 338-789. In some embodiments, the at least one SOCS1-targeting TAL effector domain binds to a target DNA sequence that is 100% identical to one of SEQ ID NOs: 23-200 or 56-187 and the at least one ZC3H12A-targeting TAL effector domain binds to a target DNA sequence that is 100% identical to one of SEQ ID NOs: 331-797 or 338-789.

In some embodiments, the protein-based gene-regulating system comprises at least two Talen fusion proteins, wherein at least one Talen fusion protein comprises a PTPN2-targeting TAL effector domain and at least one Talen fusion protein comprises a ZC3H12A-targeting TAL effector domain. In some embodiments, the at least one PTPN2-targeting TAL effector domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one PTPN2-targeting TAL effector domain binds to a target DNA sequence that is at least 95%, 96%, 97%, 98%, or 99% identical to a DNA sequence defined by a set of genomic coordinates shown in Table 9 or Table 10 and the at least one ZC3H12A-targeting TAL effector domain binds to a target DNA sequence that is at least 95%, 96%, 97%, 98%, or 99% identical to a DNA sequence defined by a set of genomic coordinates shown in Table 11 or Table 12. In some embodiments, the at least one PTPN2-targeting TAL effector domain binds to a target DNA sequence that is 100% identical to a DNA sequence defined by a set of genomic coordinates shown in Table 9 or Table 10 and the at least one ZC3H12A-targeting TAL effector domain binds to a target DNA sequence that is 100% identical to a DNA sequence defined by a set of genomic coordinates shown in Table 11 or Table 12.

In some embodiments, the at least one PTPN2-targeting TAL effector domain binds to a target DNA sequence that is at least 95%, 96%, 97%, 98%, or 99% identical to one of SEQ ID NOs: 201-327 or 201-314 and the at least one ZC3H12A-targeting TAL effector domain binds to a target DNA sequence that is at least 95%, 96%, 97%, 98%, or 99% identical to one of SEQ ID NOs: 331-797 or 338-789. In some embodiments, the at least one PTPN2-targeting TAL effector domain binds to a target DNA sequence that is 100% identical to one of SEQ ID NOs: 201-327 or 201-314 and the at least one ZC3H12A-targeting TAL effector domain binds to a target DNA sequence that is 100% identical to one of SEQ ID NOs: 331-797 or 338-789.

In some embodiments, the protein-based gene-regulating system comprises at least two Talen fusion proteins, wherein at least one Talen fusion protein comprises a CBLB-targeting TAL effector domain and at least one Talen fusion protein comprises a PTPN2-targeting TAL effector domain. In some embodiments, the at least one CBLB-targeting TAL effector domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one PTPN2-targeting TAL effector domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one CBLB-targeting TAL effector domain binds to a target DNA sequence that is 100% identical to a DNA sequence in the CBLB gene (SEQ ID NO: 7) or the Cblb gene (SEQ ID NO: 8) and the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4).

In some embodiments, the at least one CBLB-targeting TAL effector domain binds to a target DNA sequence that is at least 95%, 96%, 97%, 98%, or 99% identical to a DNA sequence defined by a set of genomic coordinates shown in Table 17 or Table 18 and the at least one PTPN2-targeting TAL effector domain binds to a target DNA sequence that is at least 95%, 96%, 97%, 98%, or 99% identical to a DNA sequence defined by a set of genomic coordinates shown in Table 9 or Table 10. In some embodiments, the at least one CBLB-targeting TAL effector domain binds to a target DNA sequence that is 100% identical to a DNA sequence defined by a set of genomic coordinates shown in Table 17 or Table 18 and the at least one PTPN2-targeting TAL effector domain binds to a target DNA sequence that is 100% identical to a DNA sequence defined by a set of genomic coordinates shown in Table 9 or Table 10.

In some embodiments, the at least one CBLB-targeting TAL effector domain binds to a target DNA sequence that is at least 95%, 96%, 97%, 98%, or 99% identical to one of SEQ ID NOs: 798-823 or 798-808 and the at least one PTPN2-targeting TAL effector domain binds to a target DNA sequence that is at least 95%, 96%, 97%, 98%, or 99% identical to one of SEQ ID NOs: 201-327 or 201-314. In some embodiments, the at least one CBLB-targeting TAL effector domain binds to a target DNA sequence that is 100% identical to one of SEQ ID NOs: 798-823 or 798-808 and the at least one PTPN2-targeting TAL effector domain binds to a target DNA sequence that is 100% identical to one of SEQ ID NOs: 201-327 or 201-314.

In some embodiments, the protein-based gene-regulating system comprises at least two Talen fusion proteins, wherein at least one Talen fusion protein comprises a CBLB-targeting TAL effector domain and at least one Talen fusion protein comprises a ZC3H12A-targeting TAL effector domain. In some embodiments, the at least one CBLB-targeting TAL effector domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one CBLB-targeting TAL effector domain binds to a target DNA sequence that is 100% identical to a DNA sequence in the CBLB gene (SEQ ID NO: 7) or the Cblb gene (SEQ ID NO: 8) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one CBLB-targeting TAL effector domain binds to a target DNA sequence that is at least 95%, 96%, 97%, 98%, or 99% identical to a DNA sequence defined by a set of genomic coordinates shown in Table 17 or Table 18 and the at least one ZC3H12A-targeting TAL effector domain binds to a target DNA sequence that is at least 95%, 96%, 97%, 98%, or 99% identical to a DNA sequence defined by a set of genomic coordinates shown in Table 11 or Table 12. In some embodiments, the at least one CBLB-targeting TAL effector domain binds to a target DNA sequence that is 100% identical to a DNA sequence defined by a set of genomic coordinates shown in Table 17 or Table 18 and the at least one ZC3H12A-targeting TAL effector domain binds to a target DNA sequence that is 100% identical to a DNA sequence defined by a set of genomic coordinates shown in Table 11 or Table 12.

In some embodiments, the at least one CBLB-targeting TAL effector domain binds to a target DNA sequence that is at least 95%, 96%, 97%, 98%, or 99% identical to one of SEQ ID NOs: 798-823 or 798-808 and the at least one ZC3H12A-targeting TAL effector domain binds to a target DNA sequence that is at least 95%, 96%, 97%, 98%, or 99% identical to one of SEQ ID NOs: 331-797 or 338-789. In some embodiments, the at least one CBLB-targeting TAL effector domain binds to a target DNA sequence that is 100% identical to one of SEQ ID NOs: 798-823 or 798-808 and the at least one ZC3H12A-targeting TAL effector domain binds to a target DNA sequence that is 100% identical to one of SEQ ID NOs: 331-797 or 338-789.

In some embodiments, the protein-based gene-regulating system comprises at least two Talen fusion proteins, wherein at least one Talen fusion protein comprises a SOCS1-targeting TAL effector domain and at least one Talen fusion protein comprises a CBLB-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one CBLB-targeting TAL effector domain binds to a target DNA sequence that is at least 95%, 96%, 97%, 98%, or 99% identical to a DNA sequence in the CBLB gene (SEQ ID NO: 7) or the Cblb gene (SEQ ID NO: 8). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one CBLB-targeting TAL effector domain binds to a target DNA sequence that is 100% identical to a DNA sequence in the CBLB gene (SEQ ID NO: 7) or the Cblb gene (SEQ ID NO: 8).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 4 or Table 5 and the at least one CBLB-targeting TAL effector domain binds to a target DNA sequence that is at least 95%, 96%, 97%, 98%, or 99% identical to a DNA sequence defined by a set of genomic coordinates shown in Table 17 or Table 18. In some embodiments, the at least one SOCS1-targeting TAL effector domain binds to a target DNA sequence that is 100% identical to a DNA sequence defined by a set of genomic coordinates shown in Table 4 or Table 5 and the at least one CBLB-targeting TAL effector domain binds to a target DNA sequence that is 100% identical to a DNA sequence defined by a set of genomic coordinates shown in Table 17 or Table 18.

In some embodiments, the at least one SOCS1-targeting TAL effector domain binds to a target DNA sequence that is at least 95%, 96%, 97%, 98%, or 99% identical to one of SEQ ID NOs: 23-200 or 56-187 and the at least one CBLB-targeting TAL effector domain binds to a target DNA sequence that is at least 95%, 96%, 97%, 98%, or 99% identical to one of SEQ ID NOs: 798-823 or 798-808. In some embodiments, the at least one SOCS1-targeting TAL effector domain binds to a target DNA sequence that is 100% identical to one of SEQ ID NOs: 23-200 or 56-187 and the at least one CBLB-targeting TAL effector domain binds to a target DNA sequence that is 100% identical to one of SEQ ID NOs: 798-823 or 798-808.

In some embodiments, the protein-based gene-regulating system comprises at least two Talen fusion proteins, wherein at least one Talen fusion protein comprises a RC3H1-targeting TAL effector domain and at least one Talen fusion protein comprises a PTPN2-targeting TAL effector domain. In some embodiments, the at least one RC3H1-targeting TAL effector domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one PTPN2-targeting TAL effector domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one RC3H1-targeting TAL effector domain binds to a target DNA sequence that is 100% identical to a DNA sequence in the RC3H1 gene (SEQ ID NO: 9) or the Rc3h1 gene (SEQ ID NO: 10) and the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4).

In some embodiments, the at least one RC3H1-targeting TAL effector domain binds to a target DNA sequence that is at least 95%, 96%, 97%, 98%, or 99% identical to a DNA sequence defined by a set of genomic coordinates shown in Table 19 or Table 20 and the at least one PTPN2-targeting TAL effector domain binds to a target DNA sequence that is at least 95%, 96%, 97%, 98%, or 99% identical to a DNA sequence defined by a set of genomic coordinates shown in Table 9 or Table 10. In some embodiments, the at least one RC3H1-targeting TAL effector domain binds to a target DNA sequence that is 100% identical to a DNA sequence defined by a set of genomic coordinates shown in Table 19 or Table 20 and the at least one PTPN2-targeting TAL effector domain binds to a target DNA sequence that is 100% identical to a DNA sequence defined by a set of genomic coordinates shown in Table 9 or Table 10.

In some embodiments, the at least one RC3H1-targeting TAL effector domain binds to a target DNA sequence that is at least 95%, 96%, 97%, 98%, or 99% identical to one of SEQ ID NOs: 824-844 or 824-836 and the at least one PTPN2-targeting TAL effector domain binds to a target DNA sequence that is at least 95%, 96%, 97%, 98%, or 99% identical to one of SEQ ID NOs: 201-327 or 201-314. In some embodiments, the at least one RC3H1-targeting TAL effector domain binds to a target DNA sequence that is 100% identical to one of SEQ ID NOs: 824-844 or 824-836 and the at least one PTPN2-targeting TAL effector domain binds to a target DNA sequence that is 100% identical to one of SEQ ID NOs: 201-327 or 201-314.

In some embodiments, the protein-based gene-regulating system comprises at least two Talen fusion proteins, wherein at least one Talen fusion protein comprises a RC3H1-targeting TAL effector domain and at least one Talen fusion protein comprises a ZC3H12A-targeting TAL effector domain. In some embodiments, the at least one RC3H1-targeting TAL effector domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one RC3H1-targeting TAL effector domain binds to a target DNA sequence that is 100% identical to a DNA sequence in the RC3H1 gene (SEQ ID NO: 9) or the Rc3h1 gene (SEQ ID NO: 10) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one RC3H1-targeting TAL effector domain binds to a target DNA sequence that is at least 95%, 96%, 97%, 98%, or 99% identical to a DNA sequence defined by a set of genomic coordinates shown in Table 19 or Table 20 and the at least one ZC3H12A-targeting TAL effector domain binds to a target DNA sequence that is at least 95%, 96%, 97%, 98%, or 99% identical to a DNA sequence defined by a set of genomic coordinates shown in Table 11 or Table 12. In some embodiments, the at least one RC3H1-targeting TAL effector domain binds to a target DNA sequence that is 100% identical to a DNA sequence defined by a set of genomic coordinates shown in Table 19 or Table 20 and the at least one ZC3H12A-targeting TAL effector domain binds to a target DNA sequence that is 100% identical to a DNA sequence defined by a set of genomic coordinates shown in Table 11 or Table 12.

In some embodiments, the at least one RC3H1-targeting TAL effector domain binds to a target DNA sequence that is at least 95%, 96%, 97%, 98%, or 99% identical to one of SEQ ID NOs: 824-844 or 824-836 and the at least one ZC3H12A-targeting TAL effector domain binds to a target DNA sequence that is at least 95%, 96%, 97%, 98%, or 99% identical to one of SEQ ID NOs: 331-797 or 338-789. In some embodiments, the at least one RC3H1-targeting TAL effector domain binds to a target DNA sequence that is 100% identical to one of SEQ ID NOs: 824-844 or 824-836 and the at least one ZC3H12A-targeting TAL effector domain binds to a target DNA sequence that is 100% identical to one of SEQ ID NOs: 331-797 or 338-789.

In some embodiments, the protein-based gene-regulating system comprises at least two Talen fusion proteins, wherein at least one Talen fusion protein comprises a SOCS1-targeting TAL effector domain and at least one Talen fusion protein comprises a RC3H1-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one RC3H1-targeting TAL effector domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one RC3H1-targeting TAL effector domain binds to a target DNA sequence that is 100% identical to a DNA sequence in the RC3H1 gene (SEQ ID NO: 9) or the Rc3h1 gene (SEQ ID NO: 10).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 4 or Table 5 and the at least one RC3H1-targeting TAL effector domain binds to a target DNA sequence that is at least 95%, 96%, 97%, 98%, or 99% identical to a DNA sequence defined by a set of genomic coordinates shown in Table 19 or Table 20. In some embodiments, the at least one SOCS1-targeting TAL effector domain binds to a target DNA sequence that is 100% identical to a DNA sequence defined by a set of genomic coordinates shown in Table 4 or Table 5 and the at least one RC3H1-targeting TAL effector domain binds to a target DNA sequence that is 100% identical to a DNA sequence defined by a set of genomic coordinates shown in Table 19 or Table 20.

In some embodiments, the protein-based gene-regulating system comprises at least two Talen fusion proteins, wherein at least one Talen fusion protein comprises a CBLB-targeting TAL effector domain and at least one Talen fusion protein comprises a RC3H1-targeting TAL effector domain. In some embodiments, the at least one CBLB-targeting TAL effector domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one RC3H1-targeting TAL effector domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10). In some embodiments, the at least one CBLB-targeting TAL effector domain binds to a target DNA sequence that is 100% identical to a DNA sequence in the CBLB gene (SEQ ID NO: 7) or the Cblb gene (SEQ ID NO: 8) and the at least one RC3H1-targeting TAL effector domain binds to a target DNA sequence that is 100% identical to a DNA sequence in the RC3H1 gene (SEQ ID NO: 9) or the Rc3h1 gene (SEQ ID NO: 10).

In some embodiments, the at least one CBLB-targeting TAL effector domain binds to a target DNA sequence that is at least 95%, 96%, 97%, 98%, or 99% identical to a DNA sequence defined by a set of genomic coordinates shown in Table 17 or Table 18 and the at least one RC3H1-targeting TAL effector domain binds to a target DNA sequence that is at least 95%, 96%, 97%, 98%, or 99% identical to a DNA sequence defined by a set of genomic coordinates shown in Table 19 or Table 20. In some embodiments, the at least one CBLB-targeting TAL effector domain binds to a target DNA sequence that is 100% identical to a DNA sequence defined by a set of genomic coordinates shown in Table 17 or Table 18 and the at least one RC3H1-targeting TAL effector domain binds to a target DNA sequence that is 100% identical to a DNA sequence defined by a set of genomic coordinates shown in Table 19 or Table 20.

In some embodiments, the at least one CBLB-targeting TAL effector domain binds to a target DNA sequence that is at least 95%, 96%, 97%, 98%, or 99% identical to one of SEQ ID NOs: 798-823 or 798-808 and the at least one RC3H1-targeting TAL effector domain binds to a target DNA sequence that is at least 95%, 96%, 97%, 98%, or 99% identical to one of SEQ ID NOs: 824-844 or 824-836. In some embodiments, the at least one CBLB-targeting TAL effector domain binds to a target DNA sequence that is 100% identical to one of SEQ ID NOs: 798-823 or 798-808 and the at least one RC3H1-targeting TAL effector domain binds to a target DNA sequence that is 100% identical to one of SEQ ID NOs: 824-844 or 824-836.

In some embodiments, the protein-based gene-regulating system comprises at least two Talen fusion proteins, wherein at least one Talen fusion protein comprises a NFKBIA-targeting TAL effector domain and at least one Talen fusion protein comprises a PTPN2-targeting TAL effector domain. In some embodiments, the at least one NFKBIA-targeting TAL effector domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12) and the at least one PTPN2-targeting TAL effector domain binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one NFKBIA-targeting TAL effector domain binds to a target DNA sequence that is 100% identical to a DNA sequence in the NFKBIA gene (SEQ ID NO: 11) or the Nfkbia gene (SEQ ID NO: 12) and the at least one PTPN2-targeting TAL effector domain binds to a target DNA sequence that is 100% identical to a DNA sequence in the PTPN2 gene (SEQ ID NO: 3) or the Ptpn2 gene (SEQ ID NO: 4).

In some embodiments, the at least one NFKBIA-targeting TAL effector domain binds to a target DNA sequence that is at least 95%, 96%, 97%, 98%, or 99% identical to a DNA sequence defined by a set of genomic coordinates shown in Table 21 or Table 22 and the at least one PTPN2-targeting TAL effector domain binds to a target DNA sequence that is at least 95%, 96%, 97%, 98%, or 99% identical to a DNA sequence defined by a set of genomic coordinates shown in Table 9 or Table 10. In some embodiments, the at least one NFKBIA-targeting TAL effector domain binds to a target DNA sequence that is 100% identical to a DNA sequence defined by a set of genomic coordinates shown in Table 21 or Table 22 and the at least one PTPN2-targeting TAL effector domain binds to a target DNA sequence that is 100% identical to a DNA sequence defined by a set of genomic coordinates shown in Table 9 or Table 10.

In some embodiments, the at least one NFKBIA-targeting TAL effector domain binds to a target DNA sequence that is at least 95%, 96%, 97%, 98%, or 99% identical to one of SEQ ID NOs: 845-875 or 845-856 and the at least one PTPN2-targeting TAL effector domain binds to a target DNA sequence that is at least 95%, 96%, 97%, 98%, or 99% identical to one of SEQ ID NOs: 201-327 or 201-314. In some embodiments, the at least one NFKBIA-targeting TAL effector domain binds to a target DNA sequence that is 100% identical to one of SEQ ID NOs: 845-875 or 845-856 and the at least one PTPN2-targeting TAL effector domain binds to a target DNA sequence that is 100% identical to one of SEQ ID NOs: 201-327 or 201-314.

In some embodiments, the protein-based gene-regulating system comprises at least two Talen fusion proteins, wherein at least one Talen fusion protein comprises a NFKBIA-targeting TAL effector domain and at least one Talen fusion protein comprises a ZC3H12A-targeting TAL effector domain. In some embodiments, the at least one NFKBIA-targeting TAL effector domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12) and the at least one ZC3H12A-targeting TAL effector domain binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one NFKBIA-targeting TAL effector domain binds to a target DNA sequence that is 100% identical to a DNA sequence in the NFKBIA gene (SEQ ID NO: 11) or the Nfkbia gene (SEQ ID NO: 12) and the at least one ZC3H12A-targeting TAL effector domain binds to a target DNA sequence that is 100% identical to a DNA sequence in the ZC3H12A gene (SEQ ID NO: 5) or the Zc3h12a gene (SEQ ID NO: 6).

In some embodiments, the at least one NFKBIA-targeting TAL effector domain binds to a target DNA sequence that is at least 95%, 96%, 97%, 98%, or 99% identical to a DNA sequence defined by a set of genomic coordinates shown in Table 21 or Table 22 and the at least one ZC3H12A-targeting TAL effector domain binds to a target DNA sequence that is at least 95%, 96%, 97%, 98%, or 99% identical to a DNA sequence defined by a set of genomic coordinates shown in Table 11 or Table 12. In some embodiments, the at least one NFKBIA-targeting TAL effector domain binds to a target DNA sequence that is 100% identical to a DNA sequence defined by a set of genomic coordinates shown in Table 21 or Table 22 and the at least one ZC3H12A-targeting TAL effector domain binds to a target DNA sequence that is 100% identical to a DNA sequence defined by a set of genomic coordinates shown in Table 11 or Table 12.

In some embodiments, the at least one NFKBIA-targeting TAL effector domain binds to a target DNA sequence that is at least 95%, 96%, 97%, 98%, or 99% identical to one of SEQ ID NOs: 845-875 or 845-856 and the at least one ZC3H12A-targeting TAL effector domain binds to a target DNA sequence that is at least 95%, 96%, 97%, 98%, or 99% identical to one of SEQ ID NOs: 331-797 or 338-789. In some embodiments, the at least one NFKBIA-targeting TAL effector domain binds to a target DNA sequence that is 100% identical to one of SEQ ID NOs: 845-875 or 845-856 and the at least one ZC3H12A-targeting TAL effector domain binds to a target DNA sequence that is 100% identical to one of SEQ ID NOs: 331-797 or 338-789.

In some embodiments, the protein-based gene-regulating system comprises at least two Talen fusion proteins, wherein at least one Talen fusion protein comprises a SOCS1-targeting TAL effector domain and at least one Talen fusion protein comprises a NFKBIA-targeting TAL effector domain. In some embodiments, the at least one SOCS1-targeting TAL effector domain binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one NFKBIA-targeting TAL effector domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one SOCS1-targeting TAL effector domain binds to a target DNA sequence that is 100% identical to a DNA sequence in the SOCS1 gene (SEQ ID NO: 1) or the Socs1 gene (SEQ ID NO: 2) and the at least one NFKBIA-targeting TAL effector domain binds to a target DNA sequence that is 100% identical to a DNA sequence in the NFKBIA gene (SEQ ID NO: 11) or the Nfkbia gene (SEQ ID NO: 12).

In some embodiments, the at least one SOCS1-targeting TAL effector domain binds to a target DNA sequence that is at least 95%, 96%, 97%, 98%, or 99% identical to a DNA sequence defined by a set of genomic coordinates shown in Table 4 or Table 5 and the at least one NFKBIA-targeting TAL effector domain binds to a target DNA sequence that is at least 95%, 96%, 97%, 98%, or 99% identical to a DNA sequence defined by a set of genomic coordinates shown in Table 21 or Table 22. In some embodiments, the at least one SOCS1-targeting TAL effector domain binds to a target DNA sequence that is 100% identical to a DNA sequence defined by a set of genomic coordinates shown in Table 4 or Table 5 and the at least one NFKBIA-targeting TAL effector domain binds to a target DNA sequence that is 100% identical to a DNA sequence defined by a set of genomic coordinates shown in Table 21 or Table 22.

In some embodiments, the at least one SOCS1-targeting TAL effector domain binds to a target DNA sequence that is at least 95%, 96%, 97%, 98%, or 99% identical to one of SEQ ID NOs: 23-200 or 56-187 and the at least one NFKBIA-targeting TAL effector domain binds to a target DNA sequence that is at least 95%, 96%, 97%, 98%, or 99% identical to one of SEQ ID NOs: 845-875 or 845-856. In some embodiments, the at least one SOCS1-targeting TAL effector domain binds to a target DNA sequence that is 100% identical to one of SEQ ID NOs: 23-200 or 56-187 and the at least one NFKBIA-targeting TAL effector domain binds to a target DNA sequence that is 100% identical to one of SEQ ID NOs: 845-875 or 845-856.

In some embodiments, the protein-based gene-regulating system comprises at least two Talen fusion proteins, wherein at least one Talen fusion protein comprises a CBLB-targeting TAL effector domain and at least one Talen fusion protein comprises a NFKBIA-targeting TAL effector domain. In some embodiments, the at least one CBLB-targeting TAL effector domain binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one NFKBIA-targeting TAL effector domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one CBLB-targeting TAL effector domain binds to a target DNA sequence that is 100% identical to a DNA sequence in the CBLB gene (SEQ ID NO: 7) or the Cblb gene (SEQ ID NO: 8) and the at least one NFKBIA-targeting TAL effector domain binds to a target DNA sequence that is 100% identical to a DNA sequence in the NFKBIA gene (SEQ ID NO: 11) or the Nfkbia gene (SEQ ID NO: 12).

In some embodiments, the at least one CBLB-targeting TAL effector domain binds to a target DNA sequence that is at least 95%, 96%, 97%, 98%, or 99% identical to a DNA sequence defined by a set of genomic coordinates shown in Table 17 or Table 18 and the at least one NFKBIA-targeting TAL effector domain binds to a target DNA sequence that is at least 95%, 96%, 97%, 98%, or 99% identical to a DNA sequence defined by a set of genomic coordinates shown in Table 21 or Table 22. In some embodiments, the at least one CBLB-targeting TAL effector domain binds to a target DNA sequence that is 100% identical to a DNA sequence defined by a set of genomic coordinates shown in Table 17 or Table 18 and the at least one NFKBIA-targeting TAL effector domain binds to a target DNA sequence that is 100% identical to a DNA sequence defined by a set of genomic coordinates shown in Table 21 or Table 22.

In some embodiments, the at least one CBLB-targeting TAL effector domain binds to a target DNA sequence that is at least 95%, 96%, 97%, 98%, or 99% identical to one of SEQ ID NOs: 798-823 or 798-808 and the at least one NFKBIA-targeting TAL effector domain binds to a target DNA sequence that is at least 95%, 96%, 97%, 98%, or 99% identical to one of SEQ ID NOs: 845-875 or 845-856. In some embodiments, the at least one CBLB-targeting TAL effector domain binds to a target DNA sequence that is 100% identical to one of SEQ ID NOs: 798-823 or 798-808 and the at least one NFKBIA-targeting TAL effector domain binds to a target DNA sequence that is 100% identical to one of SEQ ID NOs: 845-875 or 845-856.

In some embodiments, the protein-based gene-regulating system comprises at least two Talen fusion proteins, wherein at least one Talen fusion protein comprises a RC3H1-targeting TAL effector domain and at least one Talen fusion protein comprises a NFKBIA-targeting TAL effector domain. In some embodiments, the at least one RC3H1-targeting TAL effector domain binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one NFKBIA-targeting TAL effector domain binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one RC3H1-targeting TAL effector domain binds to a target DNA sequence that is 100% identical to a DNA sequence in the RC3H1 gene (SEQ ID NO: 9) or the Rc3h1 gene (SEQ ID NO: 10) and the at least one NFKBIA-targeting TAL effector domain binds to a target DNA sequence that is 100% identical to a DNA sequence in the NFKBIA gene (SEQ ID NO: 11) or the Nfkbia gene (SEQ ID NO: 12).

In some embodiments, the at least one RC3H1-targeting TAL effector domain binds to a target DNA sequence that is at least 95%, 96%, 97%, 98%, or 99% identical to a DNA sequence defined by a set of genomic coordinates shown in Table 19 or Table 20 and the at least one NFKBIA-targeting TAL effector domain binds to a target DNA sequence that is at least 95%, 96%, 97%, 98%, or 99% identical to a DNA sequence defined by a set of genomic coordinates shown in Table 21 or Table 22. In some embodiments, the at least one RC3H1-targeting TAL effector domain binds to a target DNA sequence that is 100% identical to a DNA sequence defined by a set of genomic coordinates shown in Table 19 or Table 20 and the at least one NFKBIA-targeting TAL effector domain binds to a target DNA sequence that is 100% identical to a DNA sequence defined by a set of genomic coordinates shown in Table 21 or Table 22.

In some embodiments, the at least one RC3H1-targeting TAL effector domain binds to a target DNA sequence that is at least 95%, 96%, 97%, 98%, or 99% identical to one of SEQ ID NOs: 824-844 or 824-836 and the at least one NFKBIA-targeting TAL effector domain binds to a target DNA sequence that is at least 95%, 96%, 97%, 98%, or 99% identical to one of SEQ ID NOs: 845-875 or 845-856. In some embodiments, the at least one RC3H1-targeting TAL effector domain binds to a target DNA sequence that is 100% identical to one of SEQ ID NOs: 824-844 or 824-836 and the at least one NFKBIA-targeting TAL effector domain binds to a target DNA sequence that is 100% identical to one of SEQ ID NOs: 845-875 or 845-856.

### C. Combination Nucleic Acid/Protein-Based Gene-Regulating Systems

Combination gene-regulating systems comprise a site-directed modifying polypeptide and a nucleic acid guide molecule. Herein, a “site-directed modifying polypeptide” refers to a polypeptide that binds to a nucleic acid guide molecule, is targeted to a target nucleic acid sequence, (for example, an endogenous target DNA or RNA sequence) by the nucleic acid guide molecule to which it is bound, and modifies the target nucleic acid sequence (e.g., cleavage, mutation, or methylation of a target nucleic acid sequence).

A site-directed modifying polypeptide comprises two portions, a portion that binds the nucleic acid guide and an activity portion. In some embodiments, a site-directed modifying polypeptide comprises an activity portion that exhibits site-directed enzymatic activity (e.g., DNA methylation, DNA or RNA cleavage, histone acetylation, histone methylation, etc.), wherein the site of enzymatic activity is determined by the guide nucleic acid. In some cases, a site-directed modifying polypeptide comprises an activity portion that has enzymatic activity that modifies the endogenous target nucleic acid sequence (e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity). In other cases, a site-directed modifying polypeptide comprises an activity portion that has enzymatic activity that modifies a polypeptide (e.g., a histone) associated with the endogenous target nucleic acid sequence (e.g., methyltransferase activity, demethylase activity, acetyltransferase activity, deacetylase activity, kinase activity, phosphatase activity, ubiquitin ligase activity, deubiquitinating activity, adenylation activity, deadenylation activity, SUMOylating activity, deSUMOylating activity, ribosylation activity, deribosylation activity, myristoylation activity or demyristoylation activity). In some embodiments, a site-directed modifying polypeptide comprises an activity portion that modulates transcription of a target DNA sequence (e.g., to increase or decrease transcription). In some embodiments, a site-directed modifying polypeptide comprises an activity portion that modulates expression or translation of a target RNA sequence (e.g., to increase or decrease transcription).

The nucleic acid guide comprises two portions: a first portion that is complementary to, and capable of binding with, an endogenous target nucleic sequence (referred to herein as a “nucleic acid-binding segment”), and a second portion that is capable of interacting with the site-directed modifying polypeptide (referred to herein as a “protein-binding segment”). In some embodiments, the nucleic acid-binding segment and protein-binding segment of a nucleic acid guide are comprised within a single polynucleotide molecule. In some embodiments, the nucleic acid-binding segment and protein-binding segment of a nucleic acid guide are each comprised within separate polynucleotide molecules, such that the nucleic acid guide comprises two polynucleotide molecules that associate with each other to form the functional guide.

The nucleic acid guide mediates the target specificity of the combined protein/nucleic acid gene-regulating systems by specifically hybridizing with a target nucleic acid sequence. In some embodiments, the target nucleic acid sequence is an RNA sequence, such as an RNA sequence comprised within an mRNA transcript of a target gene. In some embodiments, the target nucleic acid sequence is a DNA sequence comprised within the DNA sequence of a target gene. Reference herein to a target gene encompasses the full-length DNA sequence for that particular gene which comprises a plurality of target genetic loci (i.e., portions of a particular target gene sequence (e.g., an exon or an intron)). Within each target genetic loci are shorter stretches of DNA sequences referred to herein as “target DNA sequences” that can be modified by the gene-regulating systems described herein. Further, each target genetic loci comprises a “target modification site,” which refers to the precise location of the modification induced by the gene-regulating system (e.g., the location of an insertion, a deletion, or mutation, the location of a DNA break, or the location of an epigenetic modification). The gene-regulating systems described herein may comprise 2 or more nucleic acid guides (e.g., 2, 3, 4, 5, 6, 7, 8, 9, 10, or more nucleic acid guides).

In some embodiments, the combined protein/nucleic acid gene-regulating systems comprise site-directed modifying polypeptides derived from Argonaute (Ago) proteins (e.g., T. thermophiles Ago or TtAgo). In such embodiments, the site-directed modifying polypeptide is a T. thermophiles Ago DNA endonuclease and the nucleic acid guide is a guide DNA (gDNA) (See, Swarts et al., Nature 507 (2014), 258-261). In some embodiments, the present disclosure provides a polynucleotide encoding a gDNA. In some embodiments, a gDNA-encoding nucleic acid is comprised in an expression vector, e.g., a recombinant expression vector. In some embodiments, the present disclosure provides a polynucleotide encoding a TtAgo site-directed modifying polypeptide or variant thereof. In some embodiments, the polynucleotide encoding a TtAgo site-directed modifying polypeptide is comprised in an expression vector, e.g., a recombinant expression vector.

In some embodiments, the gene editing systems described herein are CRISPR (Clustered Regularly Interspaced Short Palindromic Repeats)/Cas (CRISPR Associated) nuclease systems. In some embodiments, the CRISPR/Cas system is a Class 2 system. Class 2 CRISPR/Cas systems are divided into three types: Type II, Type V, and Type VI systems. In some embodiments, the CRISPR/Cas system is a Class 2 Type II system, utilizing the Cas9 protein. In such embodiments, the site-directed modifying polypeptide is a Cas9 DNA endonuclease (or variant thereof) and the nucleic acid guide molecule is a guide RNA (gRNA). In some embodiments, the CRISPR/Cas system is a Class 2 Type V system, utilizing the Cas12 proteins (e.g., Cas12a (also known as Cpf1), Cas12b (also known as C2c1), Cas12c (also known as C2c3), Cas12d (also known as CasY), and Cas12e (also known as CasX)). In such embodiments, the site-directed modifying polypeptide is a Cas12 DNA endonuclease (or variant thereof) and the nucleic acid guide molecule is a gRNA. In some embodiments, the CRISPR/Cas system is a Class 2 and Type VI system, utilizing the Cas13 proteins (e.g., Cas13a (also known as C2c2), Cas13b, and Cas13c). (See, Pyzocha et al., ACS Chemical Biology, 13(2), 347-356). In such embodiments, the site-directed modifying polypeptide is a Cas13 RNA riboendonuclease and the nucleic acid guide molecule is a gRNA.

A Cas polypeptide refers to a polypeptide that can interact with a gRNA molecule and, in concert with the gRNA molecule, home or localize to a target DNA or target RNA sequence. Cas polypeptides include naturally occurring Cas proteins and engineered, altered, or otherwise modified Cas proteins that differ by one or more amino acid residues from a naturally-occurring Cas sequence.

A guide RNA (gRNA) comprises two segments, a DNA-binding segment and a protein-binding segment. In some embodiments, the protein-binding segment of a gRNA is comprised in one RNA molecule and the DNA-binding segment is comprised in another separate RNA molecule. Such embodiments are referred to herein as “double-molecule gRNAs” or “two-molecule gRNA” or “dual gRNAs.” In some embodiments, the gRNA is a single RNA molecule and is referred to herein as a “single-guide RNA” or an “sgRNA.” The term “guide RNA” or “gRNA” is inclusive, referring both to two-molecule guide RNAs and sgRNAs.

The protein-binding segment of a gRNA comprises, in part, two complementary stretches of nucleotides that hybridize to one another to form a double stranded RNA duplex (dsRNA duplex), which facilitates binding to the Cas protein. The nucleic acid-binding segment (or “nucleic acid-binding sequence”) of a gRNA comprises a nucleotide sequence that is complementary to and capable of binding to a specific target nucleic acid sequence. The protein-binding segment of the gRNA interacts with a Cas polypeptide and the interaction of the gRNA molecule and site-directed modifying polypeptide results in Cas binding to the endogenous nucleic acid sequence and produces one or more modifications within or around the target nucleic acid sequence. The precise location of the target modification site is determined by both (i) base-pairing complementarity between the gRNA and the target nucleic acid sequence; and (ii) the location of a short motif, referred to as the protospacer adjacent motif (PAM), in the target DNA sequence (referred to as a protospacer flanking sequence (PFS) in target RNA sequences). The PAM/PFS sequence is required for Cas binding to the target nucleic acid sequence. A variety of PAM/PFS sequences are known in the art and are suitable for use with a particular Cas endonuclease (e.g., a Cas9 endonuclease). (See e.g., Nat Methods. 2013 November; 10(11): 1116-1121 and Sci Rep. 2014; 4: 5405). In some embodiments, the PAM sequence is located within 50 base pairs of the target modification site in a target DNA sequence. In some embodiments, the PAM sequence is located within 10 base pairs of the target modification site in a target DNA sequence. The DNA sequences that can be targeted by this method are limited only by the relative distance of the PAM sequence to the target modification site and the presence of a unique 20 base pair sequence to mediate sequence-specific, gRNA-mediated Cas binding. In some embodiments, the PFS sequence is located at the 3′ end of the target RNA sequence. In some embodiments, the target modification site is located at the 5′ terminus of the target locus. In some embodiments, the target modification site is located at the 3′ end of the target locus. In some embodiments, the target modification site is located within an intron or an exon of the target locus.

In some embodiments, the present disclosure provides a polynucleotide encoding a gRNA. In some embodiments, a gRNA-encoding nucleic acid is comprised in an expression vector, e.g., a recombinant expression vector. In some embodiments, the present disclosure provides a polynucleotide encoding a site-directed modifying polypeptide. In some embodiments, the polynucleotide encoding a site-directed modifying polypeptide is comprised in an expression vector, e.g., a recombinant expression vector.

### 1. Cas Proteins

In some embodiments, the site-directed modifying polypeptide is a Cas protein. Cas molecules of a variety of species can be used in the methods and compositions described herein, including Cas molecules derived from S. pyogenes, S. aureus, N. meningitidis, S. thermophiles, Acidovorax avenae, Actinobacillus pleuropneumoniae, Actinobacillus succinogenes, Actinobacillus suis, Actinomyces sp., Cycliphilusdenitrificans, Aminomonas paucivorans, Bacillus cereus, Bacillus smithii, Bacillus thuringiensis, Bacteroides sp., Blastopirellula marina, Bradyrhizobium sp., Brevibacillus laterospoxus, Campylobacter coli, Campylobacter jejuni, Campylobacter lari, Candidatus puniceispirillum, Clostridium cellulolyticum, Clostridium perfringens, Corynebacterium accolens, Corynebacterium diphtheria, Corynebacterium matruchotii, Dinoroseobacter shibae, Eubacterium dolichum, Gammaproteobacterium, Gluconacetobacter diazotrophicus, Haemophilus parainfluenzae, Haemophilus sputomm, Helicobacter canadensis, Helicobacter cinaedi, Helicobacter mustelae, Ilyobacter polytropus, Kingella kingae, Lactobacillus crispatus, Listeria ivanovii, Listeria monocytogenes, Listeriaceae bacterium, Methylocystis sp., Methylosinus trichosporium, Mobiluncus mulieris, Neisseria bacilliformis, Neisseria cinerea, Neisseria flavescens, Neisseria lactamica, Neisseria meningitidis, Neisseria sp., Neisseria wadsworthii, Nitrosomonas sp., Parvibaculum lavamentivorans, Pasteurella multocida, Phascolarctobacterium succinatutens, Ralstonia syzygii, Rhodopseudomonas palustris, Rhodovulum sp., Simonsiella muelleri, Sphingomonas sp., Sporolactobacillus vineae, Staphylococcus aureus, Staphylococcus lugdunensis, Streptococcus sp., Subdoligranulum sp., Tistrella mobilis, Treponema sp., or Verminephrobacter eiseniae.

In some embodiments, the Cas protein is a naturally-occurring Cas protein. In some embodiments, the Cas endonuclease is selected from the group consisting of C2C1, C2C3, Cpf1 (also referred to as Cas12a), Cas12b, Cas12c, Cas12d, Cas12e, Cas13a, Cas13b, Cas13c, Cas13d, Cas1, Cas1B, Cas2, Cas3, Cas4, Cas5, Cas6, Cas7, Cas8, Cas9 (also known as Csn1 and Csx12), Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4.

In some embodiments, the Cas protein is an endoribonuclease such as a Cas13 protein. In some embodiments, the Cas13 protein is a Cas13a (Abudayyeh et al., Nature 550 (2017), 280-284), Cas13b (Cox et al., Science (2017) 358:6336, 1019-1027), Cas13c (Cox et al., Science (2017) 358:6336, 1019-1027), or Cas13d (Zhang et al., Cell 175 (2018), 212-223) protein.

In some embodiments, the Cas protein is a wild-type or naturally occurring Cas9 protein or a Cas9 ortholog. Wild-type Cas9 is a multi-domain enzyme that uses an HNH nuclease domain to cleave the target strand of DNA and a RuvC-like domain to cleave the non-target strand. Binding of WT Cas9 to DNA based on gRNA specificity results in double-stranded DNA breaks that can be repaired by non-homologous end joining (NHEJ) or homology-directed repair (HDR). Exemplary naturally occurring Cas9 molecules are described in Chylinski et al., RNA Biology 2013 10:5, 727-737 and additional Cas9 orthologs are described in International PCT Publication No. WO 2015/071474. Such Cas9 molecules include Cas9 molecules of a cluster 1 bacterial family, cluster 2 bacterial family, cluster 3 bacterial family, cluster 4 bacterial family, cluster 5 bacterial family, cluster 6 bacterial family, a cluster 7 bacterial family, a cluster 8 bacterial family, a cluster 9 bacterial family, a cluster 10 bacterial family, a cluster 11 bacterial family, a cluster 12 bacterial family, a cluster 13 bacterial family, a cluster 14 bacterial family, a cluster 15 bacterial family, a cluster 16 bacterial family, a cluster 17 bacterial family, a cluster 18 bacterial family, a cluster 19 bacterial family, a cluster 20 bacterial family, a cluster 21 bacterial family, a cluster 22 bacterial family, a cluster 23 bacterial family, a cluster 24 bacterial family, a cluster 25 bacterial family, a cluster 26 bacterial family, a cluster 27 bacterial family, a cluster 28 bacterial family, a cluster 29 bacterial family, a cluster 30 bacterial family, a cluster 31 bacterial family, a cluster 32 bacterial family, a cluster 33 bacterial family, a cluster 34 bacterial family, a cluster 35 bacterial family, a cluster 36 bacterial family, a cluster 37 bacterial family, a cluster 38 bacterial family, a cluster 39 bacterial family, a cluster 40 bacterial family, a cluster 41 bacterial family, a cluster 42 bacterial family, a cluster 43 bacterial family, a cluster 44 bacterial family, a cluster 45 bacterial family, a cluster 46 bacterial family, a cluster 47 bacterial family, a cluster 48 bacterial family, a cluster 49 bacterial family, a cluster 50 bacterial family, a cluster 51 bacterial family, a cluster 52 bacterial family, a cluster 53 bacterial family, a cluster 54 bacterial family, a cluster 55 bacterial family, a cluster 56 bacterial family, a cluster 57 bacterial family, a cluster 58 bacterial family, a cluster 59 bacterial family, a cluster 60 bacterial family, a cluster 61 bacterial family, a cluster 62 bacterial family, a cluster 63 bacterial family, a cluster 64 bacterial family, a cluster 65 bacterial family, a cluster 66 bacterial family, a cluster 67 bacterial family, a cluster 68 bacterial family, a cluster 69 bacterial family, a cluster 70 bacterial family, a cluster 71 bacterial family, a cluster 72 bacterial family, a cluster 73 bacterial family, a cluster 74 bacterial family, a cluster 75 bacterial family, a cluster 76 bacterial family, a cluster 77 bacterial family, or a cluster 78 bacterial family.

In some embodiments, the naturally occurring Cas9 polypeptide is selected from the group consisting of SpCas9, SpCas9-HF1, SpCas9-HF2, SpCas9-HF3, SpCas9-HF4, SaCas9, FnCpf, FnCas9, eSpCas9, and NmeCas9. In some embodiments, the Cas9 protein comprises an amino acid sequence having at least 60%, 65%, 70%, 75%, 80%, 85%, 90%, 95%, 96%, 97%, 98%, 99%, or 100% sequence identity to a Cas9 amino acid sequence described in Chylinski et al., RNA Biology 2013 10:5, 727-737; Hou et al., PNAS Early Edition 2013, 1-6).

In some embodiments, the Cas polypeptide comprises one or more of the following activities:
a. a nickase activity, i.e., the ability to cleave a single strand, e.g., the non-complementary strand or the complementary strand, of a nucleic acid molecule;
b. a double stranded nuclease activity, i.e., the ability to cleave both strands of a double stranded nucleic acid and create a double stranded break, which in an embodiment is the presence of two nickase activities;
c. an endonuclease activity;
d. an exonuclease activity; and/or
e. a helicase activity, i.e., the ability to unwind the helical structure of a double stranded nucleic acid.

In some embodiments, the Cas polypeptide is fused to heterologous proteins that recruit DNA-damage signaling proteins, exonucleases, or phosphatases to further increase the likelihood or the rate of repair of the target sequence by one repair mechanism or another. In some embodiments, a WT Cas polypeptide is co-expressed with a nucleic acid repair template to facilitate the incorporation of an exogenous nucleic acid sequence by homology-directed repair.

In some embodiments, different Cas proteins (i.e., Cas9 proteins from various species) may be advantageous to use in the various provided methods in order to capitalize on various enzymatic characteristics of the different Cas proteins (e.g., for different PAM sequence preferences; for increased or decreased enzymatic activity; for an increased or decreased level of cellular toxicity; to change the balance between NHEJ, homology-directed repair, single strand breaks, double strand breaks, etc.).

In some embodiments, the Cas protein is a Cas9 protein derived from S. pyogenes and recognizes the PAM sequence motif NGG, NAG, NGA (Mali et al, Science 2013; 339(6121): 823-826). In some embodiments, the Cas protein is a Cas9 protein derived from S. thermophiles and recognizes the PAM sequence motif NGGNG and/or NNAGAAW (W=A or T) (See, e.g., Horvath et al, Science, 2010; 327(5962): 167-170, and Deveau et al, J Bacteriol 2008; 190(4): 1390-1400). In some embodiments, the Cas protein is a Cas9 protein derived from S. mutans and recognizes the PAM sequence motif NGG and/or NAAR (R=A or G) (See, e.g., Deveau et al, J BACTERIOL 2008; 190(4): 1390-1400). In some embodiments, the Cas protein is a Cas9 protein derived from S. aureus and recognizes the PAM sequence motif NNGRR (R=A or G). In some embodiments, the Cas protein is a Cas9 protein derived from S. aureus and recognizes the PAM sequence motif N GRRT (R=A or G). In some embodiments, the Cas protein is a Cas9 protein derived from S. aureus and recognizes the PAM sequence motif N GRRV (R=A or G). In some embodiments, the Cas protein is a Cas9 protein derived from N. meningitidis and recognizes the PAM sequence motif N GATT or N GCTT (R=A or G, V=A, G or C) (See, e.g., Hou et ah, PNAS 2013, 1-6). In the aforementioned embodiments, N can be any nucleotide residue, e.g., any of A, G, C or T. In some embodiments, the Cas protein is a Cas13a protein derived from Leptotrichia shahii and recognizes the PFS sequence motif of a single 3′ A, U, or C.

In some embodiments, a polynucleotide encoding a Cas protein is provided. In some embodiments, the polynucleotide encodes a Cas protein that is at least 90% identical to a Cas protein described in International PCT Publication No. WO 2015/071474 or Chylinski et al., RNA Biology 2013 10:5, 727-737. In some embodiments, the polynucleotide encodes a Cas protein that is at least 95%, 96%, 97%, 98%, or 99% identical to a Cas protein described in International PCT Publication No. WO 2015/071474 or Chylinski et al., RNA Biology 2013 10:5, 727-737. In some embodiments, the polynucleotide encodes a Cas protein that is 100% identical to a Cas protein described in International PCT Publication No. WO 2015/071474 or Chylinski et al., RNA Biology 2013 10:5, 727-737.

### 2. Cas Mutants

In some embodiments, the Cas polypeptides are engineered to alter one or more properties of the Cas polypeptide. For example, in some embodiments, the Cas polypeptide comprises altered enzymatic properties, e.g., altered nuclease activity, (as compared with a naturally occurring or other reference Cas molecule) or altered helicase activity. In some embodiments, an engineered Cas polypeptide can have an alteration that alters its size, e.g., a deletion of amino acid sequence that reduces its size without significant effect on another property of the Cas polypeptide. In some embodiments, an engineered Cas polypeptide comprises an alteration that affects PAM recognition. For example, an engineered Cas polypeptide can be altered to recognize a PAM sequence other than the PAM sequence recognized by the corresponding wild-type Cas protein.

Cas polypeptides with desired properties can be made in a number of ways, including alteration of a naturally occurring Cas polypeptide or parental Cas polypeptide, to provide a mutant or altered Cas polypeptide having a desired property. For example, one or more mutations can be introduced into the sequence of a parental Cas polypeptide (e.g., a naturally occurring or engineered Cas polypeptide). Such mutations and differences may comprise substitutions (e.g., conservative substitutions or substitutions of non-essential amino acids); insertions; or deletions. In some embodiments, a mutant Cas polypeptide comprises one or more mutations (e.g., at least 1, 2, 3, 4, 5, 10, 15, 20, 30, 40 or 50 mutations) relative to a parental Cas polypeptide.

In an embodiment, a mutant Cas polypeptide comprises a cleavage property that differs from a naturally occurring Cas polypeptide. In some embodiments, the Cas is a deactivated Cas (dCas) mutant. In such embodiments, the Cas polypeptide does not comprise any intrinsic enzymatic activity and is unable to mediate target nucleic acid cleavage. In such embodiments, the dCas may be fused with a heterologous protein that is capable of modifying the target nucleic acid in a non-cleavage based manner. For example, in some embodiments, a dCas protein is fused to transcription activator or transcription repressor domains (e.g., the Kruppel associated box (KRAB or SKD); the Mad mSIN3 interaction domain (SID or SID4X); the ERF repressor domain (ERD); the MAX-interacting protein 1 (MXI1); methyl-CpG binding protein 2 (MECP2); etc.). In some such cases, the dCas fusion protein is targeted by the sgRNA to a specific location (i.e., sequence) in the target nucleic acid and exerts locus-specific regulation such as blocking RNA polymerase binding to a promoter (which selectively inhibits transcription activator function), and/or modifying the local chromatin status (e.g., when a fusion sequence is used that modifies the target DNA or modifies a polypeptide associated with the target DNA). In some cases, the changes are transient (e.g., transcription repression or activation). In some cases, the changes are inheritable (e.g., when epigenetic modifications are made to the target DNA or to proteins associated with the target DNA, e.g., nucleosomal histones).

In some embodiments, the dCas is a dCas13 mutant (Konermann et al., Cell 173 (2018), 665-676). These dCas13 mutants can then be fused to enzymes that modify RNA, including adenosine deaminases (e.g., ADAR1 and ADAR2). Adenosine deaminases convert adenine to inosine, which the translational machinery treats like guanine, thereby creating a functional A→G change in the RNA sequence. In some embodiments, the dCas is a dCas9 mutant.

In some embodiments, the mutant Cas9 is a Cas9 nickase mutant. Cas9 nickase mutants comprise only one catalytically active domain (either the HNH domain or the RuvC domain). The Cas9 nickase mutants retain DNA binding based on gRNA specificity, but are capable of cutting only one strand of DNA resulting in a single-strand break (e.g. a “nick”). In some embodiments, two complementary Cas9 nickase mutants (e.g., one Cas9 nickase mutant with an inactivated RuvC domain, and one Cas9 nickase mutant with an inactivated HNH domain) are expressed in the same cell with two gRNAs corresponding to two respective target sequences; one target sequence on the sense DNA strand, and one on the antisense DNA strand. This dual-nickase system results in staggered double stranded breaks and can increase target specificity, as it is unlikely that two off-target nicks will be generated close enough to generate a double stranded break. In some embodiments, a Cas9 nickase mutant is co-expressed with a nucleic acid repair template to facilitate the incorporation of an exogenous nucleic acid sequence by homology-directed repair.

In some embodiments, the Cas polypeptides described herein can be engineered to alter the PAM/PFS specificity of the Cas polypeptide. In some embodiments, a mutant Cas polypeptide has a PAM/PFS specificity that is different from the PAM/PFS specificity of the parental Cas polypeptide. For example, a naturally occurring Cas protein can be modified to alter the PAM/PFS sequence that the mutant Cas polypeptide recognizes to decrease off target sites, improve specificity, or eliminate a PAM/PFS recognition requirement. In some embodiments, a Cas protein can be modified to increase the length of the PAM/PFS recognition sequence. In some embodiments, the length of the PAM recognition sequence is at least 4, 5, 6, 7, 8, 9, 10 or 15 amino acids in length. Cas polypeptides that recognize different PAM/PFS sequences and/or have reduced off-target activity can be generated using directed evolution. Exemplary methods and systems that can be used for directed evolution of Cas polypeptides are described, e.g., in Esvelt et al. Nature 2011, 472(7344): 499-503.

Exemplary Cas mutants are described in International PCT Publication No. WO 2015/161276 and Konermann et al., Cell 173 (2018), 665-676 which are incorporated herein by reference in their entireties.

3. gRNAs

The present disclosure provides guide RNAs (gRNAs) that direct a site-directed modifying polypeptide to a specific target nucleic acid sequence. A gRNA comprises a nucleic acid-targeting segment and protein-binding segment. The nucleic acid-targeting segment of a gRNA comprises a nucleotide sequence that is complementary to a sequence in the target nucleic acid sequence. As such, the nucleic acid-targeting segment of a gRNA interacts with a target nucleic acid in a sequence-specific manner via hybridization (i.e., base pairing), and the nucleotide sequence of the nucleic acid-targeting segment determines the location within the target nucleic acid that the gRNA will bind. The nucleic acid-targeting segment of a gRNA can be modified (e.g., by genetic engineering) to hybridize to any desired sequence within a target nucleic acid sequence.

The protein-binding segment of a guide RNA interacts with a site-directed modifying polypeptide (e.g. a Cas protein) to form a complex. The guide RNA guides the bound polypeptide to a specific nucleotide sequence within target nucleic acid via the above-described nucleic acid-targeting segment. The protein-binding segment of a guide RNA comprises two stretches of nucleotides that are complementary to one another and which form a double stranded RNA duplex.

In some embodiments, a gRNA comprises two separate RNA molecules. In such embodiments, each of the two RNA molecules comprises a stretch of nucleotides that are complementary to one another such that the complementary nucleotides of the two RNA molecules hybridize to form the double-stranded RNA duplex of the protein-binding segment. In some embodiments, a gRNA comprises a single RNA molecule (sgRNA).

The specificity of a gRNA for a target locus is mediated by the sequence of the nucleic acid-binding segment, which comprises about 20 nucleotides that are complementary to a target nucleic acid sequence within the target locus. In some embodiments, the corresponding target nucleic acid sequence is approximately 20 nucleotides in length. In some embodiments, the nucleic acid-binding segments of the gRNA sequences of the present disclosure are at least 90% complementary to a target nucleic acid sequence within a target locus. In some embodiments, the nucleic acid-binding segments of the gRNA sequences of the present disclosure are at least 95%, 96%, 97%, 98%, or 99% complementary to a target nucleic acid sequence within a target locus. In some embodiments, the nucleic acid-binding segments of the gRNA sequences of the present disclosure are 100% complementary to a target nucleic acid sequence within a target locus. In some embodiments, the target nucleic acid sequence is an RNA target sequence. In some embodiments, the target nucleic acid sequence is a DNA target sequence. In some embodiments, the target nucleic acid sequence is a DNA target sequence from an endogenous genes including ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.)

In some embodiments, the gene-regulating system comprises at least one gRNA molecule that comprises a SOCS1-targeting nucleic acid-binding segment (i.e., a SOCS1-targeting gRNA). In some embodiments, the nucleic acid-binding segment of the at least one SOCS1-targeting gRNA molecules binds to a target DNA sequence that is at least 95%, 96%, 97%, 98%, or 99% identical to a DNA sequence encoded by the SOCS1 gene (SEQ ID NO: 1) or the Socs1 gene (SEQ ID NO: 2). In some embodiments, the nucleic acid-binding segment of the at least one SOCS1-targeting gRNA molecule binds to a target DNA sequence that is 100% identical to a DNA sequence encoded by the SOCS1 gene (SEQ ID NO: 1) or the Socs1 gene (SEQ ID NO: 2).

In some embodiments, the nucleic acid-binding segment of the at least one SOCS1-targeting gRNA molecule binds to a target DNA sequence that is at least 95%, 96%, 97%, 98%, or 99% identical to a DNA sequence defined by a set of genomic coordinates shown in Table 3 or Table 4. In some embodiments, the nucleic acid-binding segment of the at least one SOCS1-targeting gRNA molecules binds to a target DNA sequence that is 100% identical to a DNA sequence defined by a set of genomic coordinates shown in Table 4 or Table 5. In some embodiments, the nucleic acid-binding segment of the at least one SOCS1-targeting gRNA molecules binds to a target DNA sequence that is at least 95%, 96%, 97%, 98%, or 99% identical to one of SEQ ID NOs: 23-200, 56-200 or 56-187. In some embodiments, the nucleic acid-binding segment of the at least one SOCS1-targeting gRNA molecules binds to a target DNA sequence that is 100% identical to one of SEQ ID NOs: 23-200, 56-200 or 56-187. Exemplary SOCS1 target DNA sequences are shown in Tables 26 and 27.

In some embodiments, the nucleic acid-binding segment of the at least one SOCS1-targeting gRNA molecule is encoded by a DNA sequence that is at least 95%, 96%, 97%, 98%, or 99% identical to one of SEQ ID NOs: 23-200, 56-200 or 56-187. In some embodiments, the nucleic acid-binding segment of the at least one SOCS1-targeting gRNA molecules is encoded by a DNA sequence that is 100% identical to one of SEQ ID NOs: 23-200, 56-200 or 56-187. Exemplary DNA sequences encoding the nucleic acid-binding segment of the SOCS1-targeting gRNAs are shown in Tables 26 and 27.

TABLE 26
Exemplary human SOCS1 target sequences

| | | SEQ |
|---|---|---|
| Target | Sequence | ID |
| hSOCS1_gRNA_1 | GCGGCTGCGCGCCGAGCCCG | 56 |
| hSOCS1_gRNA_2 | GGACGCCTGCGGATTCTACT | 57 |
| hSOCS1_gRNA_3 | GGCTGCCATCCAGGTGAAAG | 58 |
| hSOCS1_gRNA_4 | GCGGCTGTCGCGCACCAGGA | 59 |
| hSOCS1_gRNA_5 | TGGACGCCTGCGGATTCTAC | 60 |
| hSOCS1_gRNA_6 | GACGCCTGCGGATTCTACTG | 61 |
| hSOCS1_gRNA_7 | AGTGCTCCAGCAGCTCGAAG | 62 |
| hSOCS1_gRNA_8 | GCCGGCCGCTTTCACCTGGA | 63 |
| hSOCS1_gRNA_9 | AGTAGAATCCGCAGGCGTCC | 64 |
| hSOCS1_gRNA_10 | CGCACCAGGAAGGTGCCCAC | 65 |
| hSOCS1_gRNA_11 | GGCCGGCCTGAAAGTGCACG | 66 |
| hSOCS1_gRNA_12 | TCCGTTCGCACGCCGATTAC | 67 |
| hSOCS1_gRNA_13 | AGCGCGCTCCTGGACGCCTG | 68 |
| hSOCS1_gRNA_14 | CGGCTGCGCGCCGAGCCCGT | 69 |
| hSOCS1_gRNA_15 | ACGCCTGCGGATTCTACTGG | 70 |
| hSOCS1_gRNA_16 | CGAGGCCATCTTCACGCTAA | 71 |
| hSOCS1_gRNA_17 | TCAGGCCGGCCGCTTTCACC | 72 |
| hSOCS1_gRNA_18 | CTTAGCGTGAAGATGGCCTC | 73 |
| hSOCS1_gRNA_19 | GCCGGTAATCGGCGTGCGAA | 74 |
| hSOCS1_gRNA_20 | CTGCATTGTCGGCTGCCACC | 75 |
| hSOCS1_gRNA_21 | GTGCGCCCCGTGCACGCTCA | 76 |
| hSOCS1_gRNA_22 | GCTGTGCCGCCAGCGCATCG | 77 |
| hSOCS1_gRNA_23 | CACGCGGCGCTGGCGCAGCG | 78 |
| hSOCS1_gRNA_24 | GCTCCTGCAGCGGCCGCACG | 79 |
| hSOCS1_gRNA_25 | AGCTCTCGCGGCTGCCATCC | 80 |
| hSOCS1_gRNA_26 | TGGTGCGCGACAGCCGCCAG | 81 |
| hSOCS1_gRNA_27 | GATGGTAGCACACAACCAGG | 82 |
| hSOCS1_gRNA_28 | AGAGGCAGTCGAAGCTCTCG | 83 |
| hSOCS1_gRNA_29 | GCTGGCGGCTGTCGCGCACC | 84 |
| hSOCS1_gRNA_30 | CCGAGGCCATCTTCACGCTA | 85 |
| hSOCS1_gRNA_31 | GGGGCCCCCAGCATGCGGCG | 86 |
| hSOCS1_gRNA_32 | GCTGCTGGAGCACTACGTGG | 87 |
| hSOCS1_gRNA_33 | CGAGCTGCTGGAGCACTACG | 88 |
| hSOCS1_gRNA_34 | CGAAAAAGCAGTTCCGCTGG | 89 |
| hSOCS1_gRNA_35 | GCAGGCGTCCAGGAGCGCGC | 90 |
| hSOCS1_gRNA_36 | GGGGCCCCTGAGCGTGCACG | 91 |
| hSOCS1_gRNA_37 | GCGGCGCCGCGCCGCATGCT | 92 |
| hSOCS1_gRNA_38 | GCACGCGGCGCTGGCGCAGC | 93 |
| hSOCS1_gRNA_39 | TGGGGGCCCCTGAGCGTGCA | 94 |
| hSOCS1_gRNA_40 | CAGGAAGGTGCCCACGGGCT | 95 |
| hSOCS1_gRNA_41 | TGCGCCCCGTGCACGCTCAG | 96 |
| hSOCS1_gRNA_42 | GCCATCCAGGTGAAAGCGGC | 97 |
| hSOCS1_gRNA_43 | CACGCGCGCCAGCGCGCTCC | 98 |
| hSOCS1_gRNA_44 | GGGCCCCCAGTAGAATCCGC | 99 |
| hSOCS1_gRNA_45 | ATCCGCGTGCACTTTCAGGC | 100 |
| hSOCS1_gRNA_46 | CGAGCCCGTGGGCACCTTCC | 101 |
| hSOCS1_gRNA_47 | CCACAGCAGCAGAGCCCCGA | 102 |
| hSOCS1_gRNA_48 | AGCCAGGTTCTCGCGGCCCA | 103 |
| hSOCS1_gRNA_49 | AAAGTGCACGCGGATGCTCG | 104 |
| hSOCS1_gRNA_50 | CTCTTCCTCCTCCTCGCCCG | 105 |
| hSOCS1_gRNA_51 | GCGTGCACGGGGCGCACGAG | 106 |
| hSOCS1_gRNA_52 | AAGTGCACGCGGATGCTCGT | 107 |
| hSOCS1_gRNA_53 | CGTGCGCCCCGTGCACGCTC | 108 |
| hSOCS1_gRNA_54 | GCAGCGGCCGCACGCGGCGC | 109 |
| hSOCS1_gRNA_55 | CCTTAGCGTGAAGATGGCCT | 110 |
| hSOCS1_gRNA_56 | CAGGTTCTCGCGGCCCACGG | 111 |
| hSOCS1_gRNA_57 | GCGCACCAGGAAGGTGCCCA | 112 |
| hSOCS1_gRNA_58 | GCTGCCGGTCAAATCTGGAA | 113 |
| hSOCS1_gRNA_59 | CGGCGTGCGAACGGAATGTG | 114 |
| hSOCS1_gRNA_60 | CAGCAGCAGAGCCCCGACGG | 115 |
| hSOCS1_gRNA_61 | GGGCGAAAAAGCAGTTCCGC | 116 |
| hSOCS1_gRNA_62 | CGCACGCGGCGCTGGCGCAG | 117 |
| hSOCS1_gRNA_63 | GGATGCGAGCCAGGTTCTCG | 118 |
| hSOCS1_gRNA_64 | TGGCGGCACAGCTCCTGCAG | 119 |
| hSOCS1_gRNA_65 | GCGCCCGCGGCCGTGCCCCG | 120 |
| hSOCS1_gRNA_66 | GGCGCCGCGCCGCATGCTGG | 121 |
| hSOCS1_gRNA_67 | CGGTGGCCACGATGCGCTGG | 122 |
| hSOCS1_gRNA_68 | TGCTGTGGAGACTGCATTGT | 123 |
| hSOCS1_gRNA_69 | TAGGATGGTAGCACACAACC | 124 |
| hSOCS1_gRNA_70 | GCGGCCGTGCCCCGCGGTCC | 125 |
| hSOCS1_gRNA_71 | GAGCATCCGCGTGCACTTTC | 126 |
| hSOCS1_gRNA_72 | CGCTGCCGGTCAAATCTGGA | 127 |
| hSOCS1_gRNA_73 | CAGCGCATCGTGGCCACCGT | 128 |
| hSOCS1_gRNA_74 | GCGGATGCTCGTGGGTCCCG | 129 |
| hSOCS1_gRNA_75 | CGGCGCCGCGCCGCATGCTG | 130 |
| hSOCS1_gRNA_76 | CGGTCAAATCTGGAAGGGGA | 131 |
| hSOCS1_gRNA_77 | AGGAAGGTTCTGGCCGCCGT | 132 |
| hSOCS1_gRNA_78 | CCACGGTGGCCACGATGCGC | 133 |
| hSOCS1_gRNA_79 | CGCTGCGCCAGCGCCGCGTG | 134 |
| hSOCS1_gRNA_80 | AGGAGCTCAGGTAGTCGCGG | 135 |
| hSOCS1_gRNA_81 | GCAGCGGGGCCCCCAGCATG | 136 |
| hSOCS1_gRNA_82 | GGAAGGAGCTCAGGTAGTCG | 137 |
| hSOCS1_gRNA_83 | TCGCGGAGGACGGGGTTGAG | 138 |
| hSOCS1_gRNA_84 | CGACTGCCTCTTCGAGCTGC | 139 |
| hSOCS1_gRNA_85 | GCGCCGCGTGCGGCCGCTGC | 140 |
| hSOCS1_gRNA_86 | CACCGTGGGCCGCGAGAACC | 141 |
| hSOCS1_gRNA_87 | GTGCCCCGCGGTCCCGGCCC | 142 |
| hSOCS1_gRNA_88 | CTGCCGGTCAAATCTGGAAG | 143 |
| hSOCS1_gRNA_89 | CTTCCCCTTCCAGATTTGAC | 144 |
| hSOCS1_gRNA_90 | CTCAGGTAGTCGCGGAGGAC | 145 |
| hSOCS1_gRNA_91 | CGGGCGCTGCCGGTCAAATC | 146 |
| hSOCS1_gRNA_92 | GGAAGGTTCTGGCCGCCGTC | 147 |
| hSOCS1_gRNA_93 | GCTCAGGTAGTCGCGGAGGA | 148 |
| hSOCS1_gRNA_94 | GCGGAAGTGCGTGTCGCCGG | 149 |
| hSOCS1_gRNA_95 | GGACCGCGGGGCACGGCCGC | 150 |
| hSOCS1_gRNA_96 | GGGACCGCGGGGCACGGCCG | 151 |
| hSOCS1_gRNA_97 | GCGCGTGATGCGCCGGTAAT | 152 |
| hSOCS1_gRNA_98 | TCAGGTAGTCGCGGAGGACG | 153 |
| hSOCS1_gRNA_99 | TGCGGAAGTGCGTGTCGCCG | 154 |
| hSOCS1_gRNA_100 | GGGGCCGGGACCGCGGGGCA | 155 |
| hSOCS1_gRNA_101 | CCGTCGGGGCTCTGCTGCTG | 156 |
| hSOCS1_gRNA_102 | GAAGGTTCTGGCCGCCGTCG | 157 |
| hSOCS1_gRNA_103 | GTGTGCTACCATCCTACAGA | 158 |
| hSOCS1_gRNA_104 | GTCGCGGAGGACGGGGTTGA | 159 |
| hSOCS1_gRNA_105 | CGCTGGCGCGCGTGATGCGC | 160 |
| hSOCS1_gRNA_106 | GCGTGCACGGCGGGCGCTGC | 161 |
| hSOCS1_gRNA_107 | TCTGGAAGGGGAAGGAGCTC | 162 |
| hSOCS1_gRNA_108 | GTGCGTGTCGCCGGGGGCCG | 163 |
| hSOCS1_gRNA_109 | GGGCACGGCCGCGGGCGCGC | 164 |
| hSOCS1_gRNA_110 | GTTAATGCTGCGTGCACGGC | 165 |
| hSOCS1_gRNA_111 | GCACGGCCGCGGGCGCGCGG | 166 |
| hSOCS1_gRNA_112 | GGGGCACGGCCGCGGGCGCG | 167 |
| hSOCS1_gRNA_113 | GTGCGGAAGTGCGTGTCGCC | 168 |
| hSOCS1_gRNA_114 | GAGGAAGAGGAGGAAGGTTC | 169 |
| hSOCS1_gRNA_115 | GGCTGGCCCCTTCTGTAGGA | 170 |
| hSOCS1_gRNA_116 | GGGGCCGGGGCCGGGACCGC | 171 |
| hSOCS1_gRNA_117 | CGCGGAGGACGGGGTTGAGG | 172 |
| hSOCS1_gRNA_118 | TTTCGCCCTTAGCGTGAAGA | 173 |
| hSOCS1_gRNA_119 | GGCACGGCCGCGGGCGCGCG | 174 |
| hSOCS1_gRNA_120 | AGTCGCGGAGGACGGGGTTG | 175 |
| hSOCS1_gRNA_121 | GGGCCGGGGCCGGGACCGCG | 176 |
| hSOCS1_gRNA_122 | AAGTGCGTGTCGCCGGGGGC | 177 |
| hSOCS1_gRNA_123 | CTCCGGCTGGCCCCTTCTGT | 178 |
| hSOCS1_gRNA_124 | GGCGGCGCCGCGCCGCATGC | 179 |
| hSOCS1_gRNA_125 | AGTGCGTGTCGCCGGGGGCC | 180 |
| hSOCS1_gRNA_126 | TGTGCGGAAGTGCGTGTCGC | 181 |
| hSOCS1_gRNA_127 | GTGTCGCCGGGGGCCGGGGC | 182 |
| hSOCS1_gRNA_128 | TGTCGCCGGGGGCCGGGGCC | 183 |
| hSOCS1_gRNA_129 | GCGGTCCCGGCCCCGGCCCC | 184 |
| hSOCS1_gRNA_130 | CGCGGGGGCCGCGGGCGAGG | 185 |
| hSOCS1_gRNA_131 | CGCGGGCGAGGAGGAGGAAG | 186 |
| hSOCS1_gRNA_132 | GGGCGAGGAGGAGGAAGAGG | 187 |

TABLE 27
Exemplary murine Socs1 target sequences

| | | SEQ |
|---|---|---|
| Target | Sequence | ID |
| mSocs1_gRNA_1 | GAAGTGCACGCGGATGCTCG | 188 |
| mSocs1_gRNA_2 | AGTGCTCCAGCAGCTCGAAA | 189 |
| mSocs1_gRNA_3 | GCCGGCCGCTTCCACTTGGA | 190 |
| mSocs1_gRNA_4 | GCTGTGTCGCCAGCGCATCG | 191 |
| mSocs1_gRNA_5 | GCGACTGTCGCGCACCAAGA | 192 |
| mSocs1_gRNA_6 | GCGTGCACGGGGCGCACGAG | 193 |
| mSocs1_gRNA_7 | TCACGGAGTACCGGGTTAAG | 194 |
| mSocs1_gRNA_8 | GGACGCCTGCGGCTTCTATT | 195 |
| mSocs1_gRNA_9 | GCGCGAAGAAGCAGTTCCGT | 196 |
| mSocs1_gRNA_10 | GCTCAGCGTGAAGATGGCTT | 197 |
| mSocs1_gRNA_11 | CGAGCCCGTGGGCACCTTCT | 198 |
| mSocs1_gRNA_12 | ATCCGCGTGCACTTCCAGGC | 199 |
| mSocs1_gRNA_13 | CGCCAGGTTCTCGCGACCCA | 200 |

In some embodiments, the gene-regulating system comprises at least one gRNA molecule that comprises a PTPN2-targeting nucleic acid-binding segment (i.e., a PTPN2-targeting gRNA). In some embodiments, the nucleic acid-binding segment of the at least one PTPN2-targeting gRNA molecules binds to a target DNA sequence that is at least 95%, 96%, 97%, 98%, or 99% identical to a DNA sequence encoded by the PTPN2 gene (SEQ ID NO: 3) or the Ptpn2 gene (SEQ ID NO: 4). In some embodiments, the nucleic acid-binding segment of the at least one PTPN2-targeting gRNA molecules binds to a target DNA sequence that is 100% identical to a DNA sequence encoded by the PTPN2 gene (SEQ ID NO: 3) or the Ptpn2 gene (SEQ ID NO: 4).

In some embodiments, the nucleic acid-binding segment of the at least one PTPN2-targeting gRNA molecule binds to a target DNA sequence that is at least 95%, 96%, 97%, 98%, or 99% identical to a DNA sequence defined by a set of genomic coordinates shown in Table 9 or Table 10. In some embodiments, the nucleic acid-binding segment of the at least one PTPN2-targeting gRNA molecules binds to a target DNA sequence that is 100% identical to a DNA sequence defined by a set of genomic coordinates shown in Table 9 or Table 10. In some embodiments, the nucleic acid-binding segment of the at least one PTPN2-targeting gRNA molecules binds to a target DNA sequence that is at least 95%, 96%, 97%, 98%, or 99% identical to one of SEQ ID NOs: 201-327 or 201-314. In some embodiments, the nucleic acid-binding segment of the at least one PTPN2-targeting gRNA molecules binds to a target DNA sequence that is 100% identical to one of SEQ ID NOs: 201-327 or 201-314. Exemplary PTPN2 target DNA sequences are shown in Tables 28 and 29.

In some embodiments, the nucleic acid-binding segment of the at least one PTPN2-targeting gRNA molecule is encoded by a DNA sequence that is at least 95%, 96%, 97%, 98%, or 99% identical to one of SEQ ID NOs: 201-327 or 201-314. In some embodiments, the nucleic acid-binding segment of the at least one PTPN2-targeting gRNA molecules is encoded by a DNA sequence that is 100% identical to one of SEQ ID NOs: 201-327 or 201-314. In some embodiments, the nucleic acid-binding segment of the at least one PTPN2-targeting gRNA molecules is encoded by a DNA sequence that is at least 95%, 96%, 97%, 98%, or 99% identical to one of SEQ ID NOs: 201-327 or 201-314. In some embodiments, the nucleic acid-binding segment of the at least one PTPN2-targeting gRNA molecules is encoded by a DNA sequence that is 100% identical to one of SEQ ID NOs: 201-327 or 201-314. Exemplary DNA sequences encoding the nucleic acid-binding segment of the PTPN2-targeting gRNAs are shown in Tables 28 and 29.

TABLE 28
Exemplary human PTPN2 gRNA sequences

| Target | Sequence | SEQ ID |
|---|---|---|
| hPTPN2_gRNA_1 | CCATGCCCACCACCATCGAG | 201 |
| hPTPN2_gRNA_2 | TCTACGGAAACGTATTCGAG | 202 |
| hPTPN2_gRNA_3 | TTTAGTATATTGAGAACTTG | 203 |
| hPTPN2_gRNA_4 | GCACTACAGTGGATCACCGC | 204 |
| hPTPN2_gRNA_5 | TGTCATGCTGAACCGCATTG | 205 |
| hPTPN2_gRNA_6 | GGAAACTTGGCCACTCTATG | 206 |
| hPTPN2_gRNA_7 | GTATTTGAAATTATTAATGC | 207 |
| hPTPN2_gRNA_8 | CAGTTTAGTTGACATAGAAG | 208 |
| hPTPN2_gRNA_9 | GGGTCTGAATAAGACCCATT | 209 |
| hPTPN2_gRNA_10 | CCATGACTATCCTCATAGAG | 210 |
| hPTPN2_gRNA_11 | CCATGACTATCCTCATAGAG | 211 |
| hPTPN2_gRNA_12 | CTCTTCGAACTCCCGCTCGA | 212 |
| hPTPN2_gRNA_13 | GAACCCTGACCATGGGCCTG | 213 |
| hPTPN2_gRNA_14 | GCTCCTTGAACCCTGACCAT | 214 |
| hPTPN2_gRNA_15 | AGTTGGATACTCAGCGTCGC | 215 |
| hPTPN2_gRNA_16 | CCGCTCGATGGTGGTGGGCA | 216 |
| hPTPN2_gRNA_17 | CAGAAATGGCAGCATGTGTT | 217 |
| hPTPN2_gRNA_18 | GCACTACAGTGGATCACCGC | 218 |
| hPTPN2_gRNA_19 | GGTAGACACTTGTCTTGTTT | 219 |
| hPTPN2_gRNA_20 | TGGCAGCATGTGTTAGGAAG | 220 |
| hPTPN2_gRNA_21 | AGGCCCATGGTCAGGGTTCA | 221 |
| hPTPN2_gRNA_22 | GTTCAGCATGACAACTGCTT | 222 |
| hPTPN2_gRNA_23 | CAATGGAGGAGAACAGTGAG | 223 |
| hPTPN2_gRNA_24 | CTCTTCTATGTCAACTAAAC | 224 |
| hPTPN2_gRNA_25 | AGTGGATCACCGCAGGCCCA | 225 |
| hPTPN2_gRNA_26 | CTGACAGGTGACCGATGTAC | 226 |
| hPTPN2_gRNA_27 | AACTCCCGCTCGATGGTGGT | 227 |
| hPTPN2_gRNA_28 | GTCTCCCTGATCCATCCAGT | 228 |
| hPTPN2_gRNA_29 | TAGAGGAAAGTCCTGTACAT | 229 |
| hPTPN2_gRNA_30 | ATGTATGGAAAGGATGGTAA | 230 |
| hPTPN2_gRNA_31 | GCCCAATGCCTGCACTACAG | 231 |
| hPTPN2_gRNA_32 | CGAGCGGGAGTTCGAAGAGT | 232 |
| hPTPN2_gRNA_33 | TCACCGCAGGCCCATGGTCA | 233 |
| hPTPN2_gRNA_34 | CAGTTTAGTTGACATAGAAG | 234 |
| hPTPN2_gRNA_35 | CCATGCCCACCACCATCGAG | 235 |
| hPTPN2_gRNA_36 | GCCAAACCATAAGCCAGAAA | 236 |
| hPTPN2_gRNA_37 | CCGATTCTTTCTCCACAATG | 237 |
| hPTPN2_gRNA_38 | TTCGAACTCCCGCTCGATGG | 238 |
| hPTPN2_gRNA_39 | AGTGCAGGCATTGGGCGCTC | 239 |
| hPTPN2_gRNA_40 | GGAAACTTGGCCACTCTATG | 240 |
| hPTPN2_gRNA_41 | ATCCACTGTAGTGCAGGCAT | 241 |
| hPTPN2_gRNA_42 | CACTCTATGAGGATAGTCAT | 242 |
| hPTPN2_gRNA_43 | CCACTCTATGAGGATAGTCA | 243 |
| hPTPN2_gRNA_44 | TCCACTGTAGTGCAGGCATT | 244 |
| hPTPN2_gRNA_45 | AAGTTCTTTCCATCGTTTCT | 245 |
| hPTPN2_gRNA_46 | TCGCTGGCAGCCGCTGTACT | 246 |
| hPTPN2_gRNA_47 | GAACTCCCGCTCGATGGTGG | 247 |
| hPTPN2_gRNA_48 | AGGATGGTAAAGGCACCAAC | 248 |
| hPTPN2_gRNA_49 | AAAGGGAGATTCTAGTATAC | 249 |
| hPTPN2_gRNA_50 | AGAATTTAGGATGTATGGAA | 250 |
| hPTPN2_gRNA_51 | GGGTCTGAATAAGACCCATT | 251 |
| hPTPN2_gRNA_52 | GGCACCAACTGGATGGATCA | 252 |
| hPTPN2_gRNA_53 | CTCTAAAATGCAAGATACAA | 253 |
| hPTPN2_gRNA_54 | GTATTTGAAATTATTAATGC | 254 |
| hPTPN2_gRNA_55 | CCTTTCTTGCAGATGGAAAA | 255 |
| hPTPN2_gRNA_56 | CTGCACCTTCTGAGCTGTGG | 256 |
| hPTPN2_gRNA_57 | ATGCTGCCATTTCTGGCTTA | 257 |
| hPTPN2_gRNA_58 | TTTCTTTAAACAGCATCTCT | 258 |
| hPTPN2_gRNA_59 | AGACATGGAATGCAGAATGC | 259 |
| hPTPN2_gRNA_60 | AGGCACCAACTGGATGGATC | 260 |
| hPTPN2_gRNA_61 | TAATGACTGAAAAATACAAT | 261 |
| hPTPN2_gRNA_62 | GAATGCAGAATGCAGGAAAT | 262 |
| hPTPN2_gRNA_63 | TTTAGGATGTATGGAAAGGA | 263 |
| hPTPN2_gRNA_64 | CTAACACATGCTGCCATTTC | 264 |
| hPTPN2_gRNA_65 | TCATACATGGCTATAATAGA | 265 |
| hPTPN2_gRNA_66 | ACGATGGAAAGAACTTTCTA | 266 |
| hPTPN2_gRNA_67 | ACGTATTCGAGAGGACAGAA | 267 |
| hPTPN2_gRNA_68 | GCGGTGATCCACTGTAGTGC | 268 |
| hPTPN2_gRNA_69 | TATTAATGCTGGATGTTAAA | 269 |
| hPTPN2_gRNA_70 | GAGATGCTGTTTAAAGAAAC | 270 |
| hPTPN2_gRNA_71 | CAGCAAGAATTTAGGATGTA | 271 |
| hPTPN2_gRNA_72 | TTGACATAGAAGAGGCACAA | 272 |
| hPTPN2_gRNA_73 | GATTCAGGGACTCCAAAATC | 273 |
| hPTPN2_gRNA_74 | CTCACTTTCATTATACTACC | 274 |
| hPTPN2_gRNA_75 | TTTAGTATATTGAGAACTTG | 275 |
| hPTPN2_gRNA_76 | AGGGACTCCAAAATCTGGCC | 276 |
| hPTPN2_gRNA_77 | AGGTTAAATGTGCACAGTAC | 277 |
| hPTPN2_gRNA_78 | ATCACCGCAGGCCCATGGTC | 278 |
| hPTPN2_gRNA_79 | AGCATCTCTTGGTCATCTGT | 279 |
| hPTPN2_gRNA_80 | GAAGGAGCAAAATGTATAAA | 280 |
| hPTPN2_gRNA_81 | GCCATTTCTGGCTTATGGTT | 281 |
| hPTPN2_gRNA_82 | CTGGATGGATCAGGGAGACA | 282 |
| hPTPN2_gRNA_83 | AAATACAATGGGAACAGAAT | 283 |
| hPTPN2_gRNA_84 | ATAATGACTGAAAAATACAA | 284 |
| hPTPN2_gRNA_85 | CATGCCCACCACCATCGAGC | 285 |
| hPTPN2_gRNA_86 | AACATGAGAAAATACCGAAT | 286 |
| hPTPN2_gRNA_87 | AGAAATGAAGCTGGTGATTC | 287 |
| hPTPN2_gRNA_88 | CCGCATTGTGGAGAAAGAAT | 288 |
| hPTPN2_gRNA_89 | GAAATGAAGCTGGTGATTCA | 289 |
| hPTPN2_gRNA_90 | TTGTTTAAAGTGAGAGAATC | 290 |
| hPTPN2_gRNA_91 | CCGCGACTCACCAAGTACAG | 291 |
| hPTPN2_gRNA_92 | GAACATGAGAAAATACCGAA | 292 |
| hPTPN2_gRNA_93 | TATACTACCTGGCCAGATTT | 293 |
| hPTPN2_gRNA_94 | TATGAGAATCTCAGTTGATC | 294 |
| hPTPN2_gRNA_95 | TCAACTGAGATTCTCATACA | 295 |
| hPTPN2_gRNA_96 | TGAGAATCTCAGTTGATCTG | 296 |
| hPTPN2_gRNA_97 | ATGAGAATCTCAGTTGATCT | 297 |
| hPTPN2_gRNA_98 | TGGTAAAGGCACCAACTGGA | 298 |
| hPTPN2_gRNA_99 | TGTCATGCTGAACCGCATTG | 299 |
| hPTPN2_gRNA_100 | TTTGGTGAATGATCAAAGGC | 300 |
| hPTPN2_gRNA_101 | ATGAAAGTGAGATATTGTTC | 301 |
| hPTPN2_gRNA_102 | TATTTCCTCATAGTGCTCTA | 302 |
| hPTPN2_gRNA_103 | AGAAGGAGCAAAATGTATAA | 303 |
| hPTPN2_gRNA_104 | TTTGTTTGGTGAATGATCAA | 304 |
| hPTPN2_gRNA_105 | TCTACGGAAACGTATTCGAG | 305 |
| hPTPN2_gRNA_106 | AAAGGCCACCACAGCTCAGA | 306 |
| hPTPN2_gRNA_107 | AGGTGCAGCAGATGAAACAG | 307 |
| hPTPN2_gRNA_108 | GGCTCCTTGAACCCTGACCA | 308 |
| hPTPN2_gRNA_109 | AAGGAGTTACATCTTAACAC | 309 |
| hPTPN2_gRNA_110 | TAAAATGCAAGATACAATGG | 310 |
| hPTPN2_gRNA_111 | ACAAGTGTCTACCAGAGAGA | 311 |
| hPTPN2_gRNA_112 | GCGCTCTGGCACCTTCTCTC | 312 |
| hPTPN2_gRNA_113 | CTGCTGCACCTTCTGAGCTG | 313 |
| hPTPN2_gRNA_114 | TCTTCCCTACCTAGAAACGA | 314 |

TABLE 29
Exemplary murine Ptpn2 gRNA sequences

| Target | Sequence | SEQ ID |
|---|---|---|
| mPTPN2_gRNA_1 | AATCTGGCCAGGTGGTATAA | 315 |
| mPTPN2_gRNA_2 | AATATGAGAAAGTATCGAAT | 316 |
| mPTPN2_gRNA_3 | ATCACTGCAGGTCCATGGTC | 317 |
| mPTPN2_gRNA_4 | ATGTGCACAGTACTGGCCAA | 318 |
| mPTPN2_gRNA_5 | GGCAGCATGTGTTCGGAAGT | 319 |
| mPTPN2_gRNA_6 | AAGAAGTTTAGAAATGAAGC | 320 |
| mPTPN2_gRNA_7 | GCCACACCATGAGCCAGAAA | 321 |
| mPTPN2_gRNA_8 | CCTTTCTTGCAGATGGAAAA | 322 |
| mPTPN2_gRNA_9 | GTACTTTGCTCCTTCTATTA | 323 |
| mPTPN2_gRNA_10 | AGAAATGAAGCTGGTGACTC | 324 |
| mPTPN2_gRNA_11 | GTTTAGCATGACAACTGCTT | 325 |
| mPTPN2_gRNA_12 | GCCCGATGCCCGCACTGCAA | 326 |
| mPTPN2_gRNA_13 | TGACAGAGAAATGGTGTTTA | 327 |

In some embodiments, the gene-regulating system comprises at least one gRNA molecule that comprises a ZC3H12A-targeting nucleic acid-binding segment (i.e., a ZC3H12A-targeting gRNA). In some embodiments, the nucleic acid-binding segment of the at least one ZC3H12A-targeting gRNA molecules binds to a target DNA sequence that is at least 95%, 96%, 97%, 98%, or 99% identical to a DNA sequence encoded by the ZC3H12A gene (SEQ ID NO: 5) or the Zc3h12a gene (SEQ ID NO: 6). In some embodiments, the nucleic acid-binding segment of the at least one ZC3H12A-targeting gRNA molecules binds to a target DNA sequence that is 100% identical to a DNA sequence encoded by the ZC3H12A gene (SEQ ID NO: 5) or the Zc3h12a gene (SEQ ID NO: 6).

In some embodiments, the nucleic acid-binding segment of the at least one ZC3H12A-targeting gRNA molecule binds to a target DNA sequence that is at least 95%, 96%, 97%, 98%, or 99% identical to a DNA sequence defined by a set of genomic coordinates shown in Table 11 or Table 12. In some embodiments, the nucleic acid-binding segment of the at least one ZC3H12A-targeting gRNA molecules binds to a target DNA sequence that is 100% identical to a DNA sequence defined by a set of genomic coordinates shown in Table 11 or Table 12. In some embodiments, the nucleic acid-binding segment of the at least one ZC3H12A-targeting gRNA molecules binds to a target DNA sequence that is at least 95%, 96%, 97%, 98%, or 99% identical to one of SEQ ID NOs: 331-797, 338-797 or 338-789. In some embodiments, the nucleic acid-binding segment of the at least one ZC3H12A-targeting gRNA molecules binds to a target DNA sequence that is 100% identical to one of SEQ ID NOs: 331-797, 338-797 or 338-789. Exemplary ZC3H12A target DNA sequences are shown in Tables 30 and 31.

In some embodiments, the nucleic acid-binding segment of the at least one ZC3H12A-targeting gRNA molecule is encoded by a DNA sequence that is at least 95%, 96%, 97%, 98%, or 99% identical to one of SEQ ID NOs: 331-797, 338-797 or 338-789. In some embodiments, the nucleic acid-binding segment of the at least one ZC3H12A-targeting gRNA molecules is encoded by a DNA sequence that is 100% identical to one of SEQ ID NOs: 331-797, 338-797 or 338-789. Exemplary DNA sequences encoding the nucleic acid-binding segment of the ZC3H12A-targeting gRNAs are shown in Tables 30 and 31.

TABLE 30
Exemplary human ZC3H12A gRNA sequences

| Target | Sequence | SEQ ID |
|---|---|---|
| hZC3H12A_gRNA_1 | AAGCTGGCCTACGAGTCTGA | 338 |
| hZC3H12A_gRNA_2 | GCGGGACTAGAGGGAGCTGA | 339 |
| hZC3H12A_gRNA_3 | CAGCTCCCTCTAGTCCCGCG | 340 |
| hZC3H12A_gRNA_4 | CAGGACGCTGTGGATCTCCG | 341 |
| hZC3H12A_gRNA_5 | AACATACTTGTCATTGACGA | 342 |
| hZC3H12A_gRNA_6 | CTCACCTGTGATGGGCACGT | 343 |
| hZC3H12A_gRNA_7 | AACACGGGACAGCCACCGAG | 344 |
| hZC3H12A_gRNA_8 | CGACAGATTCATTGTGAAGC | 345 |
| hZC3H12A_gRNA_9 | ACACCATCACGACGCGTGGG | 346 |
| hZC3H12A_gRNA_10 | TCCCAGCCATGGGAACAAGG | 347 |
| hZC3H12A_gRNA_11 | GGAGTGGAAGCGCTTCATCG | 348 |
| hZC3H12A_gRNA_12 | TTAGGGGTGCCACCACCCCG | 349 |
| hZC3H12A_gRNA_13 | GACACATACCGTGACCTCCA | 350 |
| hZC3H12A_gRNA_14 | CCGGCCCAGTGGGTCATCAG | 351 |
| hZC3H12A_gRNA_15 | CCTGGAACTGCAGATGAAGG | 352 |
| hZC3H12A_gRNA_16 | GTCCTCTCCCTCCCAGCCAT | 353 |
| hZC3H12A_gRNA_17 | TCCCCAGGGTCCCGCCAAGA | 354 |
| hZC3H12A_gRNA_18 | AGTGAGCAGTGCAGCCTGGA | 355 |
| hZC3H12A_gRNA_19 | TGTCCTCTCCCTCCCAGCCA | 356 |
| hZC3H12A_gRNA_20 | CTGGACTGGGATGAAGGTGA | 357 |
| hZC3H12A_gRNA_21 | GGGGTGGGCCCGGCTCACCA | 358 |
| hZC3H12A_gRNA_22 | CACCACCCCGCGGGACTAGA | 359 |
| hZC3H12A_gRNA_23 | CTGCTGCCACTGCCCCCGCT | 360 |
| hZC3H12A_gRNA_24 | CGGCCCGACGTGCCCATCAC | 361 |
| hZC3H12A_gRNA_25 | CACTGCCCCCGCTAGGTGCG | 362 |
| hZC3H12A_gRNA_26 | ATACACGCTGGCCTGCTCCT | 363 |
| hZC3H12A_gRNA_27 | CAAACACTGTGATGTCTGTG | 364 |
| hZC3H12A_gRNA_28 | GCGGGACCCTGGGGATGCCT | 365 |
| hZC3H12A_gRNA_29 | GCGGGAGCGCCAGACCTCAC | 366 |
| hZC3H12A_gRNA_30 | AGGACAGGCTTCTCTCCACA | 367 |
| hZC3H12A_gRNA_31 | GCAGACACCAACACGGTGCT | 368 |
| hZC3H12A_gRNA_32 | CCACCACCCCGCGGGACTAG | 369 |
| hZC3H12A_gRNA_33 | ATCCCCAGGGTCCCGCCAAG | 370 |
| hZC3H12A_gRNA_34 | CCTGGAGGAAGGAGCAGCCT | 371 |
| hZC3H12A_gRNA_35 | AGAGCCAGATGTCGGAACTT | 372 |
| hZC3H12A_gRNA_36 | ATGACCCACTGGGCCGGCAC | 373 |
| hZC3H12A_gRNA_37 | GCAGCTTTGGGCCCACAGAC | 374 |
| hZC3H12A_gRNA_38 | ACTCTCTGTTAGCAGAGAGC | 375 |
| hZC3H12A_gRNA_39 | CCAGGAAGGAAATGCACCTA | 376 |
| hZC3H12A_gRNA_40 | AGGCACCACTCACCTGTGAT | 377 |
| hZC3H12A_gRNA_41 | CTGGGCCCGTGCCGGCCCAG | 378 |
| hZC3H12A_gRNA_42 | CAGCCAGCTGCTGGGGGTCC | 379 |
| hZC3H12A_gRNA_43 | TCCACTCCTGCCGCTCGCCT | 380 |
| hZC3H12A_gRNA_44 | CGTCCAGGCAGACACCAACA | 381 |
| hZC3H12A_gRNA_45 | CCCACCCACATCAGTCCTTC | 382 |
| hZC3H12A_gRNA_46 | GCCAGCTCTTGACCCGGCCT | 383 |
| hZC3H12A_gRNA_47 | CTGCCCTCCTTTTCCTCTTC | 384 |
| hZC3H12A_gRNA_48 | CCAGCCCCACCATGAGTCTG | 385 |
| hZC3H12A_gRNA_49 | GCCGATTCTTCCACCCAGAG | 386 |
| hZC3H12A_gRNA_50 | CTCCCAGAAGAGGAAAAGGA | 387 |
| hZC3H12A_gRNA_51 | GTGGGGCAGGGCAGGCAGCC | 388 |
| hZC3H12A_gRNA_52 | GGGTCAAGAGCTGGCCGCTG | 389 |
| hZC3H12A_gRNA_53 | ATGCCCCCTGATGACCCACT | 390 |
| hZC3H12A_gRNA_54 | AGCCTTCTCTGCCTTTGGCC | 391 |
| hZC3H12A_gRNA_55 | CTCTGCCTTTGGCCGGGCCA | 392 |
| hZC3H12A_gRNA_56 | GGAACCCAGCCTGCCCTCCC | 393 |
| hZC3H12A_gRNA_57 | GGCAGGAGCCTCGCACCTAG | 394 |
| hZC3H12A_gRNA_58 | TCCCAGACCAGCACATCCTG | 395 |
| hZC3H12A_gRNA_59 | GTGAGCAGTGCAGCCTGGAT | 396 |
| hZC3H12A_gRNA_60 | GAGCCAGATGTCGGAACTTT | 397 |
| hZC3H12A_gRNA_61 | GGCCGATGGCAAGCCTTGCT | 398 |
| hZC3H12A_gRNA_62 | AGGAGCCTCGCACCTAGCGG | 399 |
| hZC3H12A_gRNA_63 | AGGTCCCCAAGAGGAAAACA | 400 |
| hZC3H12A_gRNA_64 | CGCTGAGGAGGCCTCGGCCC | 401 |
| hZC3H12A_gRNA_65 | GAGGACAGCCACAGCCGTCA | 402 |
| hZC3H12A_gRNA_66 | CAGCCCCACCATGAGTCTGT | 403 |
| hZC3H12A_gRNA_67 | ACCCCCCAGAGCCCCAAGCA | 404 |
| hZC3H12A_gRNA_68 | GAGGCACCACTCACCTGTGA | 405 |
| hZC3H12A_gRNA_69 | CCAAGAGGAAAACAGGGCAC | 406 |
| hZC3H12A_gRNA_70 | GTACGTCTCCCAGGATTGCC | 407 |
| hZC3H12A_gRNA_71 | CACAGCCTCCACCAGGTGCG | 408 |
| hZC3H12A_gRNA_72 | GATCTCGGCAGCCAGCTGCT | 409 |
| hZC3H12A_gRNA_73 | CAGCCTTCTCTGCCTTTGGC | 410 |
| hZC3H12A_gRNA_74 | CAGAAGTGACACTTACCTCA | 411 |
| hZC3H12A_gRNA_75 | GCTGGCCGCTGAGGAGGCCT | 412 |
| hZC3H12A_gRNA_76 | CAGCTCCCTCTAGTCCCGCG | 413 |
| hZC3H12A_gRNA_77 | CGGGGTGGGCCCGGCTCACC | 414 |
| hZC3H12A_gRNA_78 | GACACATACCGTGACCTCCA | 415 |
| hZC3H12A_gRNA_79 | CAGGAAGGAAATGCACCTAT | 416 |
| hZC3H12A_gRNA_80 | AGTGGCCAGCACCCATGGCC | 417 |
| hZC3H12A_gRNA_81 | CTCTCCTATTCTTCCCAGCA | 418 |
| hZC3H12A_gRNA_82 | GCCCGAGTCCAGGCAATCCT | 419 |
| hZC3H12A_gRNA_83 | CACCTTCATCTGCAGTTCCA | 420 |
| hZC3H12A_gRNA_84 | GGCACAGGCAGACAGGTGAG | 421 |
| hZC3H12A_gRNA_85 | AGCACCCATGGCCCGGCCAA | 422 |
| hZC3H12A_gRNA_86 | CCACAGGCAGCTTACTCACT | 423 |
| hZC3H12A_gRNA_87 | TTCCTGTGCTCCAAAGTGAG | 424 |
| hZC3H12A_gRNA_88 | ACCGCAGCCTTCTCTGCCTT | 425 |
| hZC3H12A_gRNA_89 | GGGAGCCAATGCCCGAGTCC | 426 |
| hZC3H12A_gRNA_90 | TTCCCAGCAAGGCTTGCCAT | 427 |
| hZC3H12A_gRNA_91 | AGCCAGATGTCGGAACTTTG | 428 |
| hZC3H12A_gRNA_92 | TACACGGGCTACAGTCCCTA | 429 |
| hZC3H12A_gRNA_93 | TCTGTGTTAGACCCTCTTGG | 430 |
| hZC3H12A_gRNA_94 | AAGCTGCCCCCAGCGCTCTG | 431 |
| hZC3H12A_gRNA_95 | CTTTGGGGGGTTCGAGGAGG | 432 |
| hZC3H12A_gRNA_96 | GGGCCGATGGCAAGCCTTGC | 433 |
| hZC3H12A_gRNA_97 | CACAGGCAGCTTACTCACTG | 434 |
| hZC3H12A_gRNA_98 | CCCAGACCAGCACATCCTGC | 435 |
| hZC3H12A_gRNA_99 | AGGCTGGGTTCCATACCATA | 436 |
| hZC3H12A_gRNA_100 | GGACTTCTAATTGCTGAGAA | 437 |
| hZC3H12A_gRNA_101 | CTCAAATTCCCACAGACTCA | 438 |
| hZC3H12A_gRNA_102 | AAAACAGGGCACAGGCAGAC | 439 |
| hZC3H12A_gRNA_103 | CCAGATGTCGGAACTTTGGG | 440 |
| hZC3H12A_gRNA_104 | CTCCCTCTAGTCCCGCGGGG | 441 |
| hZC3H12A_gRNA_105 | AGCCCCCAGTGCAGAGCCCA | 442 |
| hZC3H12A_gRNA_106 | CCTGGACGCCCAGCTTCTGC | 443 |
| hZC3H12A_gRNA_107 | CAGGGGCTGGCAGGAGCCCG | 444 |
| hZC3H12A_gRNA_108 | CCTTGTTCCCATGGCTGGGA | 445 |
| hZC3H12A_gRNA_109 | CTCATCTGCCACAGAGCGCT | 446 |
| hZC3H12A_gRNA_110 | GGCAGACACCAACACGGTGC | 447 |
| hZC3H12A_gRNA_111 | TCCCTCTTGATTCCTCTTCC | 448 |
| hZC3H12A_gRNA_112 | CCCTCCCAGCCATGGGAACA | 449 |
| hZC3H12A_gRNA_113 | GCGTAAGAAGCCACTCACTT | 450 |
| hZC3H12A_gRNA_114 | TGTGTTTCCCCCGCACCTGG | 451 |
| hZC3H12A_gRNA_115 | CTGAGACCAGTGGTCATCGA | 452 |
| hZC3H12A_gRNA_116 | GGGCAGCGACCTGAGACCAG | 453 |
| hZC3H12A_gRNA_117 | AGCAATTAGAAGTCCCTGCA | 454 |
| hZC3H12A_gRNA_118 | TGGGTGAGCTGGTGAAACAC | 455 |
| hZC3H12A_gRNA_119 | CTGTTAGCAGAGAGCTGGAC | 456 |
| hZC3H12A_gRNA_120 | CCCCTGATGACCCACTGGGC | 457 |
| hZC3H12A_gRNA_121 | GTTCACACCATCACGACGCG | 458 |
| hZC3H12A_gRNA_122 | TGTCCAGGCTGGGCCCGTGC | 459 |
| hZC3H12A_gRNA_123 | ACACAGACCTATGCCCCATC | 460 |
| hZC3H12A_gRNA_124 | GGCTGCCTGCCCTGCCCCAC | 461 |
| hZC3H12A_gRNA_125 | CCATAGGTGCATTTCCTTCC | 462 |
| hZC3H12A_gRNA_126 | CAGGCTGGGTTCCATACCAT | 463 |
| hZC3H12A_gRNA_127 | GCCCCATCACAGCCTCCACC | 464 |
| hZC3H12A_gRNA_128 | TGCCCTCCTTTTCCTCTTCT | 465 |
| hZC3H12A_gRNA_129 | GCCAGATGTCGGAACTTTGG | 466 |
| hZC3H12A_gRNA_130 | CAGGCAGACAGGTGAGAGGA | 467 |
| hZC3H12A_gRNA_131 | CCAGGAGTCTGAGCTATGAG | 468 |
| hZC3H12A_gRNA_132 | GCTCCAGGTTGGGAGCCTTA | 469 |
| hZC3H12A_gRNA_133 | CTCACCTGTGATGGGCACGT | 470 |
| hZC3H12A_gRNA_134 | AGCTGGCCTACGAGTCTGAC | 471 |
| hZC3H12A_gRNA_135 | GTGGGTGGGGGCAGTGGGTA | 472 |
| hZC3H12A_gRNA_136 | CATCTGCAGTTCCAGGGCCG | 473 |
| hZC3H12A_gRNA_137 | GATGACCCACTGGGCCGGCA | 474 |
| hZC3H12A_gRNA_138 | TGACCTCCAAGGCGAGCGGC | 475 |
| hZC3H12A_gRNA_139 | GGATCTCGGCAGCCAGCTGC | 476 |
| hZC3H12A_gRNA_140 | TCCTTTTCCTCTTCTGGGAG | 477 |
| hZC3H12A_gRNA_141 | CACGACGCGTGGGTGGCAAG | 478 |
| hZC3H12A_gRNA_142 | TTCACACCATCACGACGCGT | 479 |
| hZC3H12A_gRNA_143 | GCAGGAGCCTCGCACCTAGC | 480 |
| hZC3H12A_gRNA_144 | CACCCCTAAGGCTCCCAACC | 481 |
| hZC3H12A_gRNA_145 | TTGTCCTTGCTTGGGGCTCT | 482 |
| hZC3H12A_gRNA_146 | CAGGACAGGCTTCTCTCCAC | 483 |
| hZC3H12A_gRNA_147 | CACCTGGTGGAGGCTGTGAT | 484 |
| hZC3H12A_gRNA_148 | CGTCTGTGGGAGCCAGTCTG | 485 |
| hZC3H12A_gRNA_149 | CCCCCCAAAGTTCCGACATC | 486 |
| hZC3H12A_gRNA_150 | AGGCAGCCTGGCCAAGGAGC | 487 |
| hZC3H12A_gRNA_151 | TCTGCCTTTGGCCGGGCCAT | 488 |
| hZC3H12A_gRNA_152 | GGACAGGCTTCTCTCCACAG | 489 |
| hZC3H12A_gRNA_153 | ACGTGCCCATCACAGGTGAG | 490 |
| hZC3H12A_gRNA_154 | AGAGAGTGAGCAGTGCAGCC | 491 |
| hZC3H12A_gRNA_155 | CGCAGGAAGTTGTCCAGGCT | 492 |
| hZC3H12A_gRNA_156 | GGCTGGGAGCTCAGATCCAT | 493 |
| hZC3H12A_gRNA_157 | CAGCTCACCCAGCACCGTGT | 494 |
| hZC3H12A_gRNA_158 | CCAGCACATCCTGCGGGAAC | 495 |
| hZC3H12A_gRNA_159 | GACCTCCTTGTTCCCATGGC | 496 |
| hZC3H12A_gRNA_160 | GGGGTTCGAGGAGGAGGCCC | 497 |
| hZC3H12A_gRNA_161 | CAGAGAAGGCTGCGGTGGCT | 498 |
| hZC3H12A_gRNA_162 | GGGAGTGAGTCCAGCGTCTG | 499 |
| hZC3H12A_gRNA_163 | CAGGAGCCTCGCACCTAGCG | 500 |
| hZC3H12A_gRNA_164 | GGAGGAGGCCCTGGTGAGCC | 501 |
| hZC3H12A_gRNA_165 | CAAGCAAGGACAAAAATGGC | 502 |
| hZC3H12A_gRNA_166 | CGTCAGGGCACCCCAAGGCC | 503 |
| hZC3H12A_gRNA_167 | GCTGGCAGTGAACTGGTTTC | 504 |
| hZC3H12A_gRNA_168 | ACCTCCTTGTTCCCATGGCT | 505 |
| hZC3H12A_gRNA_169 | TCCCGCAGGATGTGCTGGTC | 506 |
| hZC3H12A_gRNA_170 | AGGGACTGTAGCCCGTGTAA | 507 |
| hZC3H12A_gRNA_171 | CCAGTACTCTCGAGGTGGAA | 508 |
| hZC3H12A_gRNA_172 | AATTCCCACAGACTCATGGT | 509 |
| hZC3H12A_gRNA_173 | CCCACCCCGAGCCCCTTACA | 510 |
| hZC3H12A_gRNA_174 | GTGCATTTCCTTCCTGGAAG | 511 |
| hZC3H12A_gRNA_175 | TCAGCGGCCAGCTCTTGACC | 512 |
| hZC3H12A_gRNA_176 | GGCCCGGCCAAAGGCAGAGA | 513 |
| hZC3H12A_gRNA_177 | ACAGAGCGCTGGGGGCAGCT | 514 |
| hZC3H12A_gRNA_178 | TCTTGATTCCTCTTCCAGGA | 515 |
| hZC3H12A_gRNA_179 | GCAAGGACAAAAATGGCCGG | 516 |
| hZC3H12A_gRNA_180 | CAGGGCAGGCAGCCTGGCCA | 517 |
| hZC3H12A_gRNA_181 | ATCTCGGCAGCCAGCTGCTG | 518 |
| hZC3H12A_gRNA_182 | CCCGCAGGATGTGCTGGTCT | 519 |
| hZC3H12A_gRNA_183 | GGCTCCAGGTTGGGAGCCTT | 520 |
| hZC3H12A_gRNA_184 | CAACACGGTGCTGGGTGAGC | 521 |
| hZC3H12A_gRNA_185 | GCAGCCGTGTCCCTATGGTA | 522 |
| hZC3H12A_gRNA_186 | TGTCCTTGCTTGGGGCTCTG | 523 |
| hZC3H12A_gRNA_187 | TCATGGTGGGGCTGGCTTCC | 524 |
| hZC3H12A_gRNA_188 | GAAGCTGGGCTATTCATCCA | 525 |
| hZC3H12A_gRNA_189 | GACCCTCTTGGCGGGACCCT | 526 |
| hZC3H12A_gRNA_190 | GGAAAGGCAGGGGGCGCGGG | 527 |
| hZC3H12A_gRNA_191 | AGGTCTGTGTTAGACCCTCT | 528 |
| hZC3H12A_gRNA_192 | CTCAGCTCCCTCTAGTCCCG | 529 |
| hZC3H12A_gRNA_193 | TAGGGACTGTAGCCCGTGTA | 530 |
| hZC3H12A_gRNA_194 | AGGGGGCATAAACCTGCAGA | 531 |
| hZC3H12A_gRNA_195 | CTCCCAGGATTGCCTGGACT | 532 |
| hZC3H12A_gRNA_196 | GGGATGAAGGTGAAGGCCGC | 533 |
| hZC3H12A_gRNA_197 | TGCAGAGCCCAGGGGCTGGC | 534 |
| hZC3H12A_gRNA_198 | GAATCGGCACTTGATCCCAT | 535 |
| hZC3H12A_gRNA_199 | CCGAGGCTGCTCCTTCCTCC | 536 |
| hZC3H12A_gRNA_200 | CCAGCTTCTGCAGGACGCTG | 537 |
| hZC3H12A_gRNA_201 | GGGCCGGCACGGGCCCAGCC | 538 |
| hZC3H12A_gRNA_202 | TGAGGTCTGGCGCTCCCGCT | 539 |
| hZC3H12A_gRNA_203 | TTGGGGTGCCCTGACGGCTG | 540 |
| hZC3H12A_gRNA_204 | ACTAGAGGGAGCTGAGGGCA | 541 |
| hZC3H12A_gRNA_205 | CCAGTTCCCGCAGGATGTGC | 542 |
| hZC3H12A_gRNA_206 | TATGCCCCCTGATGACCCAC | 543 |
| hZC3H12A_gRNA_207 | GTGAGAGGAGAGCATTGGCA | 544 |
| hZC3H12A_gRNA_208 | AGCTTACTCACTGGGGTGCT | 545 |
| hZC3H12A_gRNA_209 | ATCACAGCCTCCACCAGGTG | 546 |
| hZC3H12A_gRNA_210 | ACTGAAGTGGCCAGCACCCA | 547 |
| hZC3H12A_gRNA_211 | GCCGGCCCAGTGGGTCATCA | 548 |
| hZC3H12A_gRNA_212 | CCTGCAGAAGCTGGGCGTCC | 549 |
| hZC3H12A_gRNA_213 | GCACCGTGTTGGTGTCTGCC | 550 |
| hZC3H12A_gRNA_214 | GGCCCTGGAACTGCAGATGA | 551 |
| hZC3H12A_gRNA_215 | GTCCTTGCTTGGGGCTCTGG | 552 |
| hZC3H12A_gRNA_216 | CTCCCTGGAGAGCCAGATGT | 553 |
| hZC3H12A_gRNA_217 | AAATTCCCACAGACTCATGG | 554 |
| hZC3H12A_gRNA_218 | TCATCTGCCACAGAGCGCTG | 555 |
| hZC3H12A_gRNA_219 | AGTCGGCAGGGACACTGAAG | 556 |
| hZC3H12A_gRNA_220 | ACTCTCGAGGTGGAAAGGCA | 557 |
| hZC3H12A_gRNA_221 | CCCAGTGAGTAAGCTGCCTG | 558 |
| hZC3H12A_gRNA_222 | AGAGGGTGCAAAGAACTCTC | 559 |
| hZC3H12A_gRNA_223 | CACGATCCCGTCAGACTCGT | 560 |
| hZC3H12A_gRNA_224 | TCTGCACTGGGGGCTCCTGA | 561 |
| hZC3H12A_gRNA_225 | CAGGGGGCATAAACCTGCAG | 562 |
| hZC3H12A_gRNA_226 | TGAGGACAGCCACAGCCGTC | 563 |
| hZC3H12A_gRNA_227 | GTTTCCCCCGCACCTGGTGG | 564 |
| hZC3H12A_gRNA_228 | TTAGGGGTGCCACCACCCCG | 565 |
| hZC3H12A_gRNA_229 | ACTGGGGTGCTGGGACTTGT | 566 |
| hZC3H12A_gRNA_230 | CTCACTCCCGTACGTCTCCC | 567 |
| hZC3H12A_gRNA_231 | AGGGGCTGGCAGGAGCCCGT | 568 |
| hZC3H12A_gRNA_232 | TCCTTGTTCCCATGGCTGGG | 569 |
| hZC3H12A_gRNA_233 | GCCAAAGGCAGAGAAGGCTG | 570 |
| hZC3H12A_gRNA_234 | CACGGGCTCCTGCCAGCCCC | 571 |
| hZC3H12A_gRNA_235 | CCACAGCGTCCTGCAGAAGC | 572 |
| hZC3H12A_gRNA_236 | ACGGGCTCCTGCCAGCCCCT | 573 |
| hZC3H12A_gRNA_237 | ATGGGAGCAACGTGGCCATG | 574 |
| hZC3H12A_gRNA_238 | CCCAAGGCCGGGTCAAGAGC | 575 |
| hZC3H12A_gRNA_239 | AATTGCTGAGAAGGGGCCGA | 576 |
| hZC3H12A_gRNA_240 | GGGCAGGAGTGAGGAGGGCC | 577 |
| hZC3H12A_gRNA_241 | GGCGGGACCCTGGGGATGCC | 578 |
| hZC3H12A_gRNA_242 | GGGGCTGGCAGGAGCCCGTG | 579 |
| hZC3H12A_gRNA_243 | TTCCGACATCTGGCTCTCCA | 580 |
| hZC3H12A_gRNA_244 | GTGCTGCCCTTGCCAGCCAC | 581 |
| hZC3H12A_gRNA_245 | ACTCCTGCCGCTCGCCTTGG | 582 |
| hZC3H12A_gRNA_246 | GTGGACTTCTTCCGGAAGCT | 583 |
| hZC3H12A_gRNA_247 | CCAGTGCAGAGCCCAGGGGC | 584 |
| hZC3H12A_gRNA_248 | GGGGCAGTGGCAGCAGCTTT | 585 |
| hZC3H12A_gRNA_249 | GGGACTGTAGCCCGTGTAAG | 586 |
| hZC3H12A_gRNA_250 | CCACAGACTCATGGTGGGGC | 587 |
| hZC3H12A_gRNA_251 | AACACGGGACAGCCACCGAG | 588 |
| hZC3H12A_gRNA_252 | GCAAAGAACTCTCTGGAGGT | 589 |
| hZC3H12A_gRNA_253 | TGGGCCCGTGCCGGCCCAGT | 590 |
| hZC3H12A_gRNA_254 | CTCCTGCCGGGGCATCCTGC | 591 |
| hZC3H12A_gRNA_255 | AGGCAGACAGGTGAGAGGAA | 592 |
| hZC3H12A_gRNA_256 | AGGCAATCCTGGGAGACGTA | 593 |
| hZC3H12A_gRNA_257 | TCAGACCAGTACTCTCGAGG | 594 |
| hZC3H12A_gRNA_258 | AACATACTTGTCATTGACGA | 595 |
| hZC3H12A_gRNA_259 | GGCAGCTTGGCCGCTCTGGG | 596 |
| hZC3H12A_gRNA_260 | GAGTTCTTTGCACCCTCTGC | 597 |
| hZC3H12A_gRNA_261 | GCCACAGGCAGCTTACTCAC | 598 |
| hZC3H12A_gRNA_262 | AGGCTGCCTGCCCTGCCCCA | 599 |
| hZC3H12A_gRNA_263 | CCGGCCCAGTGGGTCATCAG | 600 |
| hZC3H12A_gRNA_264 | CTCTCGAGGTGGAAAGGCAG | 601 |
| hZC3H12A_gRNA_265 | GATTGCCTGGACTCGGGCAT | 602 |
| hZC3H12A_gRNA_266 | TCCTTGCTTGGGGCTCTGGG | 603 |
| hZC3H12A_gRNA_267 | GCAGAGAAGGCTGCGGTGGC | 604 |
| hZC3H12A_gRNA_268 | ACCGTGACCTCCAAGGCGAG | 605 |
| hZC3H12A_gRNA_269 | CAGGACGCTGTGGATCTCCG | 606 |
| hZC3H12A_gRNA_270 | AGGAAGCAGCCGTGTCCCTA | 607 |
| hZC3H12A_gRNA_271 | ACGCAGGAAGTTGTCCAGGC | 608 |
| hZC3H12A_gRNA_272 | GAGGTCCCCAAGAGGAAAAC | 609 |
| hZC3H12A_gRNA_273 | CCCCCAGCTTCTTCCCATCC | 610 |
| hZC3H12A_gRNA_274 | ATTCCCACAGACTCATGGTG | 611 |
| hZC3H12A_gRNA_275 | TCCAAGGCGAGCGGCAGGAG | 612 |
| hZC3H12A_gRNA_276 | GCTGGGAGCTCAGATCCATA | 613 |
| hZC3H12A_gRNA_277 | TGGGGGCCCAGGCATCCCCA | 614 |
| hZC3H12A_gRNA_278 | GGGTGCAAAGAACTCTCTGG | 615 |
| hZC3H12A_gRNA_279 | GCGGGACTAGAGGGAGCTGA | 616 |
| hZC3H12A_gRNA_280 | ACTGGAGAAGAAGAAGATCC | 617 |
| hZC3H12A_gRNA_281 | CCAGCTCTTGACCCGGCCTT | 618 |
| hZC3H12A_gRNA_282 | GAACTTTGGGGGGTTCGAGG | 619 |
| hZC3H12A_gRNA_283 | GAAACCAGTTCACTGCCAGC | 620 |
| hZC3H12A_gRNA_284 | ACAGCCGTCAGGGCACCCCA | 621 |
| hZC3H12A_gRNA_285 | CCACCCCGAGCCCCTTACAC | 622 |
| hZC3H12A_gRNA_286 | TCTCGGCAGCCAGCTGCTGG | 623 |
| hZC3H12A_gRNA_287 | AGAGAGCTGGACTGGGATGA | 624 |
| hZC3H12A_gRNA_288 | CCTTTCCACCTCGAGAGTAC | 625 |
| hZC3H12A_gRNA_289 | AAGCTGGCCTACGAGTCTGA | 626 |
| hZC3H12A_gRNA_290 | GTCTGTGGGAGCCAGTCTGT | 627 |
| hZC3H12A_gRNA_291 | AGACCTATGCCCCATCAGGC | 628 |
| hZC3H12A_gRNA_292 | TGGGAAGAAGCTGGGGGCCC | 629 |
| hZC3H12A_gRNA_293 | CTGTGGAGAGAAGCCTGTCC | 630 |
| hZC3H12A_gRNA_294 | GGGACTTCTAATTGCTGAGA | 631 |
| hZC3H12A_gRNA_295 | GGACTCGGGCATTGGCTCCC | 632 |
| hZC3H12A_gRNA_296 | CATCTGCCACAGAGCGCTGG | 633 |
| hZC3H12A_gRNA_297 | CTTCTGGGAGTGGAGGCTCC | 634 |
| hZC3H12A_gRNA_298 | GCCCCCAGTGCAGAGCCCAG | 635 |
| hZC3H12A_gRNA_299 | TTTGTCCTTGCTTGGGGCTC | 636 |
| hZC3H12A_gRNA_300 | GTGGGGCTGGCTTCCAGGAC | 637 |
| hZC3H12A_gRNA_301 | TCAAGAGCTGGCCGCTGAGG | 638 |
| hZC3H12A_gRNA_302 | CCTCTAGTCCCGCGGGGTGG | 639 |
| hZC3H12A_gRNA_303 | GCTCATCTGCCACAGAGCGC | 640 |
| hZC3H12A_gRNA_304 | CATGAGTCTGTGGGAATTTG | 641 |
| hZC3H12A_gRNA_305 | TGCGAGGCTCCTGCCTGATG | 642 |
| hZC3H12A_gRNA_306 | GGAGTGAGTCCAGCGTCTGT | 643 |
| hZC3H12A_gRNA_307 | TGCAAAGAACTCTCTGGAGG | 644 |
| hZC3H12A_gRNA_308 | CACAGCGTCCTGCAGAAGCT | 645 |
| hZC3H12A_gRNA_309 | CAGCTTACTCACTGGGGTGC | 646 |
| hZC3H12A_gRNA_310 | ACTGATGTGGGTGGGGGCAG | 647 |
| hZC3H12A_gRNA_311 | GCAGGATGTGCTGGTCTGGG | 648 |
| hZC3H12A_gRNA_312 | TCACAGTGTTTGTGCCATCC | 649 |
| hZC3H12A_gRNA_313 | GTTTGTGCCATCCTGGAGGA | 650 |
| hZC3H12A_gRNA_314 | TCCTGAAGGACTGATGTGGG | 651 |
| hZC3H12A_gRNA_315 | TGTTAGCAGAGAGCTGGACT | 652 |
| hZC3H12A_gRNA_316 | CAGTGTTTGTGCCATCCTGG | 653 |
| hZC3H12A_gRNA_317 | AGTCTGTCAGGGCCTCTGGG | 654 |
| hZC3H12A_gRNA_318 | TCTCGAGGTGGAAAGGCAGG | 655 |
| hZC3H12A_gRNA_319 | AGACTGGCTCCCACAGACGC | 656 |
| hZC3H12A_gRNA_320 | AGCCACTCACTTTGGAGCAC | 657 |
| hZC3H12A_gRNA_321 | TCCCAGGATTGCCTGGACTC | 658 |
| hZC3H12A_gRNA_322 | CCTGGAACTGCAGATGAAGG | 659 |
| hZC3H12A_gRNA_323 | GGGGCGCTTCCCACAGCTCC | 660 |
| hZC3H12A_gRNA_324 | CAGCCCCTGGGCTCTGCACT | 661 |
| hZC3H12A_gRNA_325 | GCGCGGGTGGGTAGTCGGCA | 662 |
| hZC3H12A_gRNA_326 | GCCCCAAGCAAGGACAAAAA | 663 |
| hZC3H12A_gRNA_327 | AGCCTGGATGGGAAGAAGCT | 664 |
| hZC3H12A_gRNA_328 | CAGCTCTTGACCCGGCCTTG | 665 |
| hZC3H12A_gRNA_329 | TAGGGGTGCCACCACCCCGC | 666 |
| hZC3H12A_gRNA_330 | TCCACTCCCAGAAGAGGAAA | 667 |
| hZC3H12A_gRNA_331 | GGAAGCGCTTCATCGAGGAG | 668 |
| hZC3H12A_gRNA_332 | GCATCCTGCTGGCAGTGAAC | 669 |
| hZC3H12A_gRNA_333 | TGGATGAATAGCCCAGCTTC | 670 |
| hZC3H12A_gRNA_334 | ACACGGGACAGCCACCGAGC | 671 |
| hZC3H12A_gRNA_335 | GGGCTCCTGAAGGACTGATG | 672 |
| hZC3H12A_gRNA_336 | CAGCCTGGATGGGAAGAAGC | 673 |
| hZC3H12A_gRNA_337 | TTTTCCTCTTCTGGGAGTGG | 674 |
| hZC3H12A_gRNA_338 | CTCCAGGTTGGGAGCCTTAG | 675 |
| hZC3H12A_gRNA_339 | GGGAGCTGAGGGCAGGGGTC | 676 |
| hZC3H12A_gRNA_340 | AGATGAAGGTGGACTTCTTC | 677 |
| hZC3H12A_gRNA_341 | TTTGGCCGGGCCATGGGTGC | 678 |
| hZC3H12A_gRNA_342 | CTCGCACCTAGCGGGGGCAG | 679 |
| hZC3H12A_gRNA_343 | CCCGTGTAAGGGGCTCGGGG | 680 |
| hZC3H12A_gRNA_344 | TGCCGGCCCAGTGGGTCATC | 681 |
| hZC3H12A_gRNA_345 | AAAGGCAGAGAAGGCTGCGG | 682 |
| hZC3H12A_gRNA_346 | AGGAGCCCGTGGGGCAGGGC | 683 |
| hZC3H12A_gRNA_347 | TAAGGGGCTCGGGGTGGGCC | 684 |
| hZC3H12A_gRNA_348 | ACACCATCACGACGCGTGGG | 685 |
| hZC3H12A_gRNA_349 | CTGGCAGGAGCCCGTGGGGC | 686 |
| hZC3H12A_gRNA_350 | CCGGCCTTGGGGTGCCCTGA | 687 |
| hZC3H12A_gRNA_351 | CTGTGTTAGACCCTCTTGGC | 688 |
| hZC3H12A_gRNA_352 | GTGATGGGCACGTCGGGCCG | 689 |
| hZC3H12A_gRNA_353 | GCCCCTGGGCTCTGCACTGG | 690 |
| hZC3H12A_gRNA_354 | CTGGGTGAGCTGGTGAAACA | 691 |
| hZC3H12A_gRNA_355 | GGCTGCTCCTTCCTCCAGGA | 692 |
| hZC3H12A_gRNA_356 | ACAGCCTCCACCAGGTGCGG | 693 |
| hZC3H12A_gRNA_357 | TGCCCGAGTCCAGGCAATCC | 694 |
| hZC3H12A_gRNA_358 | AGGTGGAAAGGCAGGGGGCG | 695 |
| hZC3H12A_gRNA_359 | CGACAGATTCATTGTGAAGC | 696 |
| hZC3H12A_gRNA_360 | GCGGGGTGGTGGCACCCCTA | 697 |
| hZC3H12A_gRNA_361 | GGCAATCCTGGGAGACGTAC | 698 |
| hZC3H12A_gRNA_362 | GCCGCTCGCCTTGGAGGTCA | 699 |
| hZC3H12A_gRNA_363 | TCACTGCCAGCAGGATGCCC | 700 |
| hZC3H12A_gRNA_364 | CCTGAAGGACTGATGTGGGT | 701 |
| hZC3H12A_gRNA_365 | GTGCGAGGCTCCTGCCTGAT | 702 |
| hZC3H12A_gRNA_366 | GCACCTGGTGGAGGCTGTGA | 703 |
| hZC3H12A_gRNA_367 | TCACAGCCTCCACCAGGTGC | 704 |
| hZC3H12A_gRNA_368 | GCCGCTCTGGGTGGAAGAAT | 705 |
| hZC3H12A_gRNA_369 | GACTAGAGGGAGCTGAGGGC | 706 |
| hZC3H12A_gRNA_370 | TCAGCTCCCTCTAGTCCCGC | 707 |
| hZC3H12A_gRNA_371 | GGAGCCTCCACTCCCAGAAG | 708 |
| hZC3H12A_gRNA_372 | AGACCCTCTTGGCGGGACCC | 709 |
| hZC3H12A_gRNA_373 | CCACCTTCATCTGCAGTTCC | 710 |
| hZC3H12A_gRNA_374 | GGGAGTGGAGGCTCCAGGTT | 711 |
| hZC3H12A_gRNA_375 | CAGTGAACTGGTTTCTGGAG | 712 |
| hZC3H12A_gRNA_376 | TCACCTGTGATGGGCACGTC | 713 |
| hZC3H12A_gRNA_377 | TGCCAGCAGGATGCCCCGGC | 714 |
| hZC3H12A_gRNA_378 | ACCCTCTTGGCGGGACCCTG | 715 |
| hZC3H12A_gRNA_379 | TGGGGGCAGCTTGGCCGCTC | 716 |
| hZC3H12A_gRNA_380 | AGGAGGAGGCCCTGGTGAGC | 717 |
| hZC3H12A_gRNA_381 | CTGGAGGTGGGAGCCATGCA | 718 |
| hZC3H12A_gRNA_382 | TCTGGAGGTGGGAGCCATGC | 719 |
| hZC3H12A_gRNA_383 | CCTGGATGGGAAGAAGCTGG | 720 |
| hZC3H12A_gRNA_384 | CCAGCCCCTGGGCTCTGCAC | 721 |
| hZC3H12A_gRNA_385 | GGAGTGGAAGCGCTTCATCG | 722 |
| hZC3H12A_gRNA_386 | TGTAGCCCGTGTAAGGGGCT | 723 |
| hZC3H12A_gRNA_387 | GGAGTGAGGAGGGCCGGGGA | 724 |
| hZC3H12A_gRNA_388 | GAGGTCACGGTATGTGTCGT | 725 |
| hZC3H12A_gRNA_389 | CTAGAGGGAGCTGAGGGCAG | 726 |
| hZC3H12A_gRNA_390 | TGGTGTGTTTCCCCCGCACC | 727 |
| hZC3H12A_gRNA_391 | CTGATGTGGGTGGGGGCAGT | 728 |
| hZC3H12A_gRNA_392 | AGGGCCGGGGAGGGCAGGCT | 729 |
| hZC3H12A_gRNA_393 | TGAGCTATGAGTGGCCCCTG | 730 |
| hZC3H12A_gRNA_394 | TCTTACGCAGGAAGTTGTCC | 731 |
| hZC3H12A_gRNA_395 | GTTCCGACATCTGGCTCTCC | 732 |
| hZC3H12A_gRNA_396 | AGGGGGCGCGGGTGGGTAGT | 733 |
| hZC3H12A_gRNA_397 | CGCTGGCCTGCTCCTTGGCC | 734 |
| hZC3H12A_gRNA_398 | GAAAGGCAGGGGGCGCGGGT | 735 |
| hZC3H12A_gRNA_399 | TAGCCCGTGTAAGGGGCTCG | 736 |
| hZC3H12A_gRNA_400 | CTGAGGGCAGGGGTCCGGTG | 737 |
| hZC3H12A_gRNA_401 | ACACAGCTTAGTATACACGC | 738 |
| hZC3H12A_gRNA_402 | CCGTCAGGGCACCCCAAGGC | 739 |
| hZC3H12A_gRNA_403 | GGCAGGGGTCCGGTGAGGTC | 740 |
| hZC3H12A_gRNA_404 | GGACTTGTAGGAGAGGATCT | 741 |
| hZC3H12A_gRNA_405 | TCCCAGCCATGGGAACAAGG | 742 |
| hZC3H12A_gRNA_406 | GACTTCTAATTGCTGAGAAG | 743 |
| hZC3H12A_gRNA_407 | GGCTCCTGAAGGACTGATGT | 744 |
| hZC3H12A_gRNA_408 | TGGCAGGAGCCCGTGGGGCA | 745 |
| hZC3H12A_gRNA_409 | AGACAGGTGAGAGGAAGGGC | 746 |
| hZC3H12A_gRNA_410 | TCGGAACTTTGGGGGGTTCG | 747 |
| hZC3H12A_gRNA_411 | GCCTGGATGGGAAGAAGCTG | 748 |
| hZC3H12A_gRNA_412 | TGAAGGACTGATGTGGGTGG | 749 |
| hZC3H12A_gRNA_413 | CTGGGGGCCCAGGCATCCCC | 750 |
| hZC3H12A_gRNA_414 | GAGCCCCCAGTGCAGAGCCC | 751 |
| hZC3H12A_gRNA_415 | GGCGCGGGTGGGTAGTCGGC | 752 |
| hZC3H12A_gRNA_416 | CCGTGTAAGGGGCTCGGGGT | 753 |
| hZC3H12A_gRNA_417 | GTCGTGATGGTGTGAACACC | 754 |
| hZC3H12A_gRNA_418 | ACGACGCGTGGGTGGCAAGC | 755 |
| hZC3H12A_gRNA_419 | GGGGGCAGTGGCAGCAGCTT | 756 |
| hZC3H12A_gRNA_420 | AGCGTGTATACTAAGCTGTG | 757 |
| hZC3H12A_gRNA_421 | GCTCCTGCCTGATGGGGCAT | 758 |
| hZC3H12A_gRNA_422 | GTCTGTCAGGGCCTCTGGGA | 759 |
| hZC3H12A_gRNA_423 | GTAGCCCGTGTAAGGGGCTC | 760 |
| hZC3H12A_gRNA_424 | AGCCCCTGGGCTCTGCACTG | 761 |
| hZC3H12A_gRNA_425 | GTGAACTGGTTTCTGGAGCG | 762 |
| hZC3H12A_gRNA_426 | CTACGAGTCTGACGGGATCG | 763 |
| hZC3H12A_gRNA_427 | AGTGAACTGGTTTCTGGAGC | 764 |
| hZC3H12A_gRNA_428 | ACGCGTGGGTGGCAAGCGGG | 765 |
| hZC3H12A_gRNA_429 | CGCGGGACTAGAGGGAGCTG | 766 |
| hZC3H12A_gRNA_430 | GGCAGGAGTGAGGAGGGCCG | 767 |
| hZC3H12A_gRNA_431 | AAGTGAGTGGCTTCTTACGC | 768 |
| hZC3H12A_gRNA_432 | CTGAAGGACTGATGTGGGTG | 769 |
| hZC3H12A_gRNA_433 | TTGCCACCCACGCGTCGTGA | 770 |
| hZC3H12A_gRNA_434 | AGGGCAGGAGTGAGGAGGGC | 771 |
| hZC3H12A_gRNA_435 | TCTTCTTCTCCAGTTCCCGC | 772 |
| hZC3H12A_gRNA_436 | AGGAGTGAGGAGGGCCGGGG | 773 |
| hZC3H12A_gRNA_437 | ACTCCCAGAAGAGGAAAAGG | 774 |
| hZC3H12A_gRNA_438 | TGAGGAGGGCCGGGGAGGGC | 775 |
| hZC3H12A_gRNA_439 | ACCTGGTGGAGGCTGTGATG | 776 |
| hZC3H12A_gRNA_440 | CAGGGCCGAGGCCTCCTCAG | 777 |
| hZC3H12A_gRNA_441 | TACTCTCGAGGTGGAAAGGC | 778 |
| hZC3H12A_gRNA_442 | TTGGGGCTCTGGGGGGTGAG | 779 |
| hZC3H12A_gRNA_443 | GCTCCTGGACCCCCAGCAGC | 780 |
| hZC3H12A_gRNA_444 | GGGGGGTGAGAGGAGAGCAT | 781 |
| hZC3H12A_gRNA_445 | CGGCCCAGTGGGTCATCAGG | 782 |
| hZC3H12A_gRNA_446 | AGGAAGGGCAGGAGTGAGGA | 783 |
| hZC3H12A_gRNA_447 | GAGGGCCGGGGAGGGCAGGC | 784 |
| hZC3H12A_gRNA_448 | TGCTGGGGGTCCAGGAGCTG | 785 |
| hZC3H12A_gRNA_449 | GCTGGGGGTCCAGGAGCTGT | 786 |
| hZC3H12A_gRNA_450 | TGAGAGGAAGGGCAGGAGTG | 787 |
| hZC3H12A_gRNA_451 | TGGGAGTGGAGGCTCCAGGT | 788 |
| hZC3H12A_gRNA_452 | GAGGAAGGGCAGGAGTGAGG | 789 |

TABLE 31
Exemplary murine Zc3h12a gRNA sequences

| Target | Sequence | SEQ ID |
|---|---|---|
| mZc3h12a_gRNA_1 | GCTGGCTGTGAACTGGTTTC | 790 |
| mZc3h12a_gRNA_2 | CTAGTTCCCGAAGGATGTGC | 791 |
| mZc3h12a_gRNA_3 | ATTGGAGACCACCACTCCGT | 792 |
| mZc3h12a_gRNA_4 | TTCCCTCCTCTGCCAGCCAT | 793 |
| mZc3h12a_gRNA_5 | CGAAGGAAGTTGTCCAGGCT | 794 |
| mZc3h12a_gRNA_6 | ATACCTGTGATAGGCACATC | 795 |
| mZc3h12a_gRNA_7 | GACTTCCTTGTTCCCATGGC | 796 |
| mZc3h12a_gRNA_8 | GGCCTTCGAATCCGACGGAG | 797 |

In some embodiments, the gene-regulating system comprises at least one gRNA molecule that comprises a CBLB-targeting nucleic acid-binding segment (i.e., a CBLB-targeting gRNA). In some embodiments, the nucleic acid-binding segment of the at least one CBLB-targeting gRNA molecules binds to a target DNA sequence that is at least 95%, 96%, 97%, 98%, or 99% identical to a DNA sequence encoded by the CBLB gene (SEQ ID NO: 7) or the Cblb gene (SEQ ID NO: 8). In some embodiments, the nucleic acid-binding segment of the at least one CBLB-targeting gRNA molecules binds to a target DNA sequence that is 100% identical to a DNA sequence encoded by the CBLB gene (SEQ ID NO: 7) or the Cblb gene (SEQ ID NO: 8).

In some embodiments, the nucleic acid-binding segment of the at least one CBLB-targeting gRNA molecule binds to a target DNA sequence that is at least 95%, 96%, 97%, 98%, or 99% identical to a DNA sequence defined by a set of genomic coordinates shown in Table 17 or Table 18. In some embodiments, the nucleic acid-binding segment of the at least one CBLB-targeting gRNA molecules binds to a target DNA sequence that is 100% identical to a DNA sequence defined by a set of genomic coordinates shown in Table 17 or Table 18. In some embodiments, the nucleic acid-binding segment of the at least one CBLB-targeting gRNA molecules binds to a target DNA sequence that is at least 95%, 96%, 97%, 98%, or 99% identical to one of SEQ ID NOs: 798-823 or 798-808. In some embodiments, the nucleic acid-binding segment of the at least one CBLB-targeting gRNA molecules binds to a target DNA sequence that is 100% identical to one of SEQ ID NOs: 798-823 or 798-808. Exemplary CBLB target DNA sequences are shown in Tables 32 and 33.

In some embodiments, the nucleic acid-binding segment of the at least one CBLB-targeting gRNA molecule is encoded by a DNA sequence that is at least 95%, 96%, 97%, 98%, or 99% identical to one of SEQ ID NOs: 798-823 or 798-808. In some embodiments, the nucleic acid-binding segment of the at least one CBLB-targeting gRNA molecules is encoded by a DNA sequence that is 100% identical to one of SEQ ID NOs: 798-823 or 798-808. Exemplary DNA sequences encoding the nucleic acid-binding segment of the CBLB-targeting gRNAs are shown in Tables 32 and 33.

TABLE 32
Exemplary human CBLB gRNA sequences

| Target | Sequence | SEQ ID |
|---|---|---|
| hCBLB_gRNA_1 | CCTTATGAAAAAGTCAAAAC | 798 |
| hCBLB_gRNA_2 | AAAATATCAAGTATATATGG | 799 |
| hCBLB_gRNA_3 | TCTAGCATCGGCATGCCAAA | 800 |
| hCBLB_gRNA_4 | TTGGAAGCTCATGGACAAAG | 801 |
| hCBLB_gRNA_5 | GATTTCCTCCTCGACCACCA | 802 |
| hCBLB_gRNA_6 | CTTCATCTCTTGGATCAAAG | 803 |
| hCBLB_gRNA_7 | AATGTATGAAGAACAGTCAC | 804 |
| hCBLB_gRNA_8 | TAAACTTACCTGAAACAGCC | 805 |
| hCBLB_gRNA_9 | AAGAATATGATGTTCCTCCC | 806 |
| hCBLB_gRNA_10 | AGCAAGCTGCCGCAGATCGC | 807 |
| hCBLB_gRNA_11 | AGTACTCATTCTCACTGAGT | 808 |

TABLE 33
Exemplary murine Chlb gRNA sequences

| Target | Sequence | SEQ ID |
|---|---|---|
| mCblb_gRNA_1 | TCTTTGTTGCAGGAGTCTGA | 809 |
| mCblb_gRNA_2 | CAGGGGCTTGTTATGAGGTA | 810 |
| mCblb_gRNA_3 | CTGATTGATGGTAGCAGGGA | 811 |
| mCblb_gRNA_4 | CCTTATCTTCAGTCACATGC | 812 |
| mCblb_gRNA_5 | TCACATGCTGGCAGAAATCA | 813 |
| mCblb_gRNA_6 | TTCTGTCGCTGTGAGATAAA | 814 |
| mCblb_gRNA_7 | ACAAGGCAGTACCTGCCACG | 815 |
| mCblb_gRNA_8 | TGTGACTCACCCGGGATACA | 816 |
| mCblb_gRNA_9 | GAGGTCCATCAGATCAGCTC | 817 |
| mCblb_gRNA_10 | ATCTCCCTGGAACTGGCCAT | 818 |
| mCblb_gRNA_11 | TGCAAAAATTGCAAAACTCA | 819 |
| mCblb_gRNA_12 | TGCACAGAACTATTGTACCA | 820 |
| mCblb_gRNA_13 | CAGATTAGTGCTTACCTTCC | 821 |
| mCblb_gRNA_14 | ATTCCGTAAAATAGAGCCCC | 822 |
| mCblb_gRNA_15 | CTGCACTCGGCTGGGACAAT | 823 |

In some embodiments, the gene-regulating system comprises at least one gRNA molecule that comprises a RC3H1-targeting nucleic acid-binding segment (i.e., a RC3H1-targeting gRNA). In some embodiments, the nucleic acid-binding segment of the at least one RC3H1-targeting gRNA molecules binds to a target DNA sequence that is at least 95%, 96%, 97%, 98%, or 99% identical to a DNA sequence encoded by the RC3H1 gene (SEQ ID NO: 9) or the Rc3h1 gene (SEQ ID NO: 10). In some embodiments, the nucleic acid-binding segment of the at least one RC3H1-targeting gRNA molecules binds to a target DNA sequence that is 100% identical to a DNA sequence encoded by the RC3H1 gene (SEQ ID NO: 9) or the Rc3h1 gene (SEQ ID NO: 10).

In some embodiments, the nucleic acid-binding segment of the at least one RC3H1-targeting gRNA molecule binds to a target DNA sequence that is at least 95%, 96%, 97%, 98%, or 99% identical to a DNA sequence defined by a set of genomic coordinates shown in Table 19 or Table 20. In some embodiments, the nucleic acid-binding segment of the at least one RC3H1-targeting gRNA molecules binds to a target DNA sequence that is 100% identical to a DNA sequence defined by a set of genomic coordinates shown in Table 19 or Table 20. In some embodiments, the nucleic acid-binding segment of the at least one RC3H1-targeting gRNA molecules binds to a target DNA sequence that is at least 95%, 96%, 97%, 98%, or 99% identical to one of SEQ ID NOs: 824-844 or 824-836. In some embodiments, the nucleic acid-binding segment of the at least one RC3H1-targeting gRNA molecules binds to a target DNA sequence that is 100% identical to one of SEQ ID NOs: 824-844 or 824-836. Exemplary RC3H1 target DNA sequences are shown in Tables 34 and 35.

In some embodiments, the nucleic acid-binding segment of the at least one RC3H1-targeting gRNA molecule is encoded by a DNA sequence that is at least 95%, 96%, 97%, 98%, or 99% identical to one of SEQ ID NOs: 824-844 or 824-836. In some embodiments, the nucleic acid-binding segment of the at least one R2H1-targeting gRNA molecules is encoded by a DNA sequence that is 100% identical to one of SEQ ID NOs: 824-844 or 824-836. Exemplary DNA sequences encoding the nucleic acid-binding segment of the RC3H1-targeting gRNAs are shown in Tables 34 and 35.

TABLE 34
Exemplary human RC3H1 gRNA sequences

| Target | Sequence | SEQ ID |
|---|---|---|
| h RC3H1_gRNA_1 | AGTCCATATGGAACCCACGG | 824 |
| h RC3H1_gRNA_2 | AGTCTGAGTGCAAATTGGGC | 825 |
| h RC3H1_gRNA_3 | TACGAATTGCACCGGACCAG | 826 |
| h RC3H1_gRNA_4 | TTAGAGGCTTGAGGAAACCG | 827 |
| h RC3H1_gRNA_5 | TTAGAACCTATGAAGCTCTG | 828 |
| h RC3H1_gRNA_6 | CCTGAATAAACTCCACCGCA | 829 |
| h RC3H1_gRNA_7 | AATTCGAAAGCCCATCAGTT | 830 |
| h RC3H1_gRNA_8 | TGGCCACAACCCAAACTGAT | 831 |
| h RC3H1_gRNA_9 | CAGCATACTCTGAGGTACGA | 832 |
| h RC3H1_gRNA_10 | TTACCTCTAGCACTGCTGAG | 833 |
| h RC3H1_gRNA_11 | TATGCAGTCCATTATTGACA | 834 |
| h RC3H1_gRNA_12 | GTAACACAGCTTATTCCGCG | 835 |
| h RC3H1_gRNA_13 | ACTTTCCCTAGCAATGCAGG | 836 |

TABLE 35
Exemplary murine Rc3h1 gRNA sequences

| Target | Sequence | SEQ ID |
|---|---|---|
| m Rc3h1_gRNA_1 | CAAATGGGCAAGCCTTACGG | 837 |
| m Rc3h1_gRNA_2 | CTCAATGTCCGTATTGATAG | 838 |
| m Rc3h1_gRNA_3 | AGTCTGAGTGCAAATTGGGC | 839 |
| m Rc3h1_gRNA_4 | CCAGATAGTGCAAATTGCTA | 840 |
| m Rc3h1_gRNA_5 | TGATAGTGGTCTGGTCAAAT | 841 |
| m Rc3h1_gRNA_6 | AATTCGAAAGCCCATCAGTT | 842 |
| m Rc3h1_gRNA_7 | GCCCATTACTTTGTGTAGTG | 843 |
| m Rc3h1_gRNA_8 | TGGCCACAGCCCAAACTGAT | 844 |

In some embodiments, the gene-regulating system comprises at least one gRNA molecule that comprises a NFKBIA-targeting nucleic acid-binding segment (i.e., a NFKBIA-targeting gRNA). In some embodiments, the nucleic acid-binding segment of the at least one NFKBIA-targeting gRNA molecule binds to a target DNA sequence that is at least 95%, 96%, 97%, 98%, or 99% identical to a DNA sequence encoded by the NFKBIA gene (SEQ ID NO: 11) or the Nfkbia gene (SEQ ID NO: 12). In some embodiments, the nucleic acid-binding segment of the at least one NFKBIA-targeting gRNA molecules binds to a target DNA sequence that is 100% identical to a DNA sequence encoded by the NFKBIA gene (SEQ ID NO: 11) or the Nfkbia gene (SEQ ID NO: 12).

In some embodiments, the nucleic acid-binding segment of the at least one NFKBIA-targeting gRNA molecule binds to a target DNA sequence that is at least 95%, 96%, 97%, 98%, or 99% identical to a DNA sequence defined by a set of genomic coordinates shown in Table 21 or Table 22. In some embodiments, the nucleic acid-binding segment of the at least one NFKBIA-targeting gRNA molecules binds to a target DNA sequence that is 100% identical to a DNA sequence defined by a set of genomic coordinates shown in Table 21 or Table 22. In some embodiments, the nucleic acid-binding segment of the at least one NFKBIA-targeting gRNA molecules binds to a target DNA sequence that is at least 95%, 96%, 97%, 98%, or 99% identical to one of SEQ ID NOs: 845-875 or 845-856. In some embodiments, the nucleic acid-binding segment of the at least one NFKBIA-targeting gRNA molecules binds to a target DNA sequence that is 100% identical to one of SEQ ID NOs: 845-875 or 845-856. Exemplary NFKBIA target DNA sequences are shown in Tables 36 and 37.

In some embodiments, the nucleic acid-binding segment of the at least one NFKBIA-targeting gRNA molecule is encoded by a DNA sequence that is at least 95%, 96%, 97%, 98%, or 99% identical to one of SEQ ID NOs: 845-875 or 845-856. In some embodiments, the nucleic acid-binding segment of the at least one NFKBIA-targeting gRNA molecules is encoded by a DNA sequence that is 100% identical to one of SEQ ID NOs: 845-875 or 845-856. Exemplary DNA sequences encoding the nucleic acid-binding segment of the NFKBIA-targeting gRNAs are shown in Tables 36 and 37.

TABLE 36
Exemplary human NFKBIA gRNA sequences

| Target | Sequence | SEQ ID |
|---|---|---|
| hNFKBIA_gRNA_1 | CGTCCGCGCCATGTTCCAGG | 845 |
| hNFKBIA_gRNA_2 | TGGTTTCAGGAGCCCTGTAA | 846 |
| hNFKBIA_gRNA_3 | ACCCGGATACAGCAGCAGCT | 847 |
| hNFKBIA_gRNA_4 | TTCCAGGGCTCCGAGCCGCG | 848 |
| hNFKBIA_gRNA_5 | CTGAAGGCTACCAACTACAA | 849 |
| hNFKBIA_gRNA_6 | GGGTATTTCCTCGAAAGTCT | 850 |
| hNFKBIA_gRNA_7 | GAGCCGCAGGAGGTGCCGCG | 851 |
| hNFKBIA_gRNA_8 | CTGAGTCAGGACTCCCACGC | 852 |
| hNFKBIA_gRNA_9 | CACTTACGAGTCCCCGTCCT | 853 |
| hNFKBIA_gRNA_10 | CTCAAATTCCTTTTGGTTTC | 854 |
| hNFKBIA_gRNA_11 | GGTTGGTGATCACAGCCAAG | 855 |
| hNFKBIA_gRNA_12 | GCAGGTTGTTCTGGAAGTTG | 856 |

TABLE 37
Exemplary murine Nfkbia gRNA sequences

| Target | Sequence | SEQ ID |
|---|---|---|
| mNfkbia_gRNA_1 | CCTCGAAAGTCTCGGAGCTC | 857 |
| mNfkbia_gRNA_2 | CTGCGTCAAGACTGCTACAC | 858 |
| mNfkbia_gRNA_3 | TGCTCACAGGCAAGATGTAG | 859 |
| mNfkbia_gRNA_4 | CCGGACAGCCCTCCACCTTG | 860 |
| mNfkbia_gRNA_5 | AGACCTACCATTGTAGTTGG | 861 |
| mNfkbia_gRNA_6 | CCAAGTGCTCCACGATGGCC | 862 |
| mNfkbia_gRNA_7 | AGCCTCTATCCACGGCTACC | 863 |
| mNfkbia_gRNA_8 | GCCCCAGGTAAGCTGGTAGG | 864 |
| mNfkbia_gRNA_9 | GCAAGCAGCGCACCTGCTGC | 865 |
| mNfkbia_gRNA_10 | TCAAGACTGCTACACTGGCC | 866 |
| mNfkbia_gRNA_11 | GCAGGTTGTTCTGGAAGTTG | 867 |
| mNfkbia_gRNA_12 | GGGTGCTGATGTCAACGCTC | 868 |
| mNfkbia_gRNA_13 | CCACGATGGCCAGGTAGCCG | 869 |
| mNfkbia_gRNA_14 | TGGTCAGCGGCTTCTCTTCG | 870 |
| mNfkbia_gRNA_15 | AATGTGGGGCTGATGTCAAC | 871 |
| mNfkbia_gRNA_16 | ATTTCAACAAGAGCGAAACC | 872 |
| mNfkbia_gRNA_17 | CACCTGACCAATGACTTCCA | 873 |
| mNfkbia_gRNA_18 | GCCCTGGAAGCAGCAGCTCA | 874 |
| mNfkbia_gRNA_19 | GCTCACAGGCAAGATGTAGA | 875 |

In some embodiments, the gene-regulating system comprises at least two gRNA molecules, wherein at least one gRNA molecule is a SOCS1-targeting gRNA molecule and at least one gRNA molecule is a PTPN2-targeting gRNA molecule. In some embodiments, the at least one SOCS1-targeting gRNA molecule binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one PTPN2-targeting gRNA molecule binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one SOCS1-targeting gRNA molecule binds to a target DNA sequence that is 100% identical to a DNA sequence in the SOCS1 gene (SEQ ID NO: 1) or the Socs1 gene (SEQ ID NO: 2) and the at least one PTPN2-targeting gRNA molecule binds to a target DNA sequence that is 100% identical to a DNA sequence in the PTPN2 gene (SEQ ID NO: 3) or the Ptpn2 gene (SEQ ID NO: 4).

In some embodiments, the at least one SOCS1-targeting gRNA molecule binds to a target DNA sequence that is at least 95%, 96%, 97%, 98%, or 99% identical to a DNA sequence defined by a set of genomic coordinates shown in Table 4 or Table 5 and the at least one PTPN2-targeting gRNA molecule binds to a target DNA sequence that is at least 95%, 96%, 97%, 98%, or 99% identical to a DNA sequence defined by a set of genomic coordinates shown in Table 9 or Table 10. In some embodiments, the at least one SOCS1-targeting gRNA molecule binds to a target DNA sequence that is 100% identical to a DNA sequence encoded by a DNA sequence defined by a set of genomic coordinates shown in Table 4 or Table 5 and the at least one PTPN2-targeting gRNA molecule binds to a target DNA sequence that is 100% identical to a DNA sequence encoded by a DNA sequence defined by a set of genomic coordinates shown in Table 9 or Table 10.

In some embodiments, the at least one SOCS1-targeting gRNA molecule binds to a target DNA sequence that is at least 95%, 96%, 97%, 98%, or 99% identical to one of SEQ ID NOs: 23-200, 56-200 or 56-187 and the at least one PTPN2-targeting gRNA molecule binds to a target DNA sequence that is at least 95%, 96%, 97%, 98%, or 99% identical to one of SEQ ID NOs: 201-327 or 201-314. In some embodiments, the at least one SOCS1-targeting gRNA molecule binds to a target DNA sequence that is 100% identical to one of SEQ ID NOs: 23-200, 56-200 or 56-187 and the at least one PTPN2-targeting gRNA molecule binds to a target DNA sequence that is 100% identical to one of SEQ ID NOs: 201-327 or 201-314.

In some embodiments, the at least one SOCS1-targeting gRNA molecule is encoded by a DNA sequence that is at least 95%, 96%, 97%, 98%, or 99% identical to one of SEQ ID NOs: 23-200, 56-200 or 56-187 and the at least one PTPN2-targeting gRNA molecule is encoded by a DNA sequence that is at least 95%, 96%, 97%, 98%, or 99% identical to one of SEQ ID NOs: 201-327 or 201-314. In some embodiments, the at least one SOCS1-targeting gRNA molecule is encoded by a DNA sequence that is 100% identical to one of SEQ ID NOs: 23-200, 56-200 or 56-187 and the at least one PTPN2-targeting gRNA molecule is encoded by a DNA sequence that is 100% identical to one of SEQ ID NOs: 201-327 or 201-314.

In some embodiments, the gene-regulating system comprises at least two gRNA molecules, wherein at least one gRNA molecule is a SOCS1-targeting gRNA molecule and at least one gRNA molecule is a ZC3H12A-targeting gRNA molecule. In some embodiments, the at least one SOCS1-targeting gRNA molecule binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one ZC3H12A-targeting gRNA molecule binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one SOCS1-targeting gRNA molecule binds to a target DNA sequence that is 100% identical to a DNA sequence in the SOCS1 gene (SEQ ID NO: 1) or the Socs1 gene (SEQ ID NO: 2) and the at least one ZC3H12A-targeting gRNA molecule binds to a target DNA sequence that is 100% identical to a DNA sequence in the ZC3H12A gene (SEQ ID NO: 5) or the Zc3h12a gene (SEQ ID NO: 6).

In some embodiments, the at least one SOCS1-targeting gRNA molecule binds to a target DNA sequence that is at least 95%, 96%, 97%, 98%, or 99% identical to a DNA sequence defined by a set of genomic coordinates shown in Table 4 or Table 5 and the at least one ZC3H12A-targeting gRNA molecule binds to a target DNA sequence that is at least 95%, 96%, 97%, 98%, or 99% identical to a DNA sequence defined by a set of genomic coordinates shown in Table 11 or Table 12. In some embodiments, the at least one SOCS1-targeting gRNA molecule binds to a target DNA sequence that is 100% identical to a DNA sequence encoded by a DNA sequence defined by a set of genomic coordinates shown in Table 4 or Table 5 and the at least one ZC3H12A-targeting gRNA molecule binds to a target DNA sequence that is 100% identical to a DNA sequence encoded by a DNA sequence defined by a set of genomic coordinates shown in Table 11 or Table 12.

In some embodiments, the at least one SOCS1-targeting gRNA molecule binds to a target DNA sequence that is at least 95%, 96%, 97%, 98%, or 99% identical to one of SEQ ID NOs: 23-200, 56-200 or 56-187 and the at least one ZC3H12A-targeting gRNA molecule binds to a target DNA sequence that is at least 95%, 96%, 97%, 98%, or 99% identical to one of SEQ ID NOs: 331-797, 338-797 or 338-789. In some embodiments, the at least one SOCS1-targeting gRNA molecule binds to a target DNA sequence that is 100% identical to one of SEQ ID NOs: 23-200, 56-200 or 56-187 and the at least one ZC3H12A-targeting gRNA molecule binds to a target DNA sequence that is 100% identical to one of SEQ ID NOs: 331-797, 338-797 or 338-789.

In some embodiments, the at least one SOCS1-targeting gRNA molecule is encoded by a DNA sequence that is at least 95%, 96%, 97%, 98%, or 99% identical to one of SEQ ID NOs: 23-200, 56-200 or 56-187 and the at least one ZC3H12A-targeting gRNA molecule is encoded by a DNA sequence that is at least 95%, 96%, 97%, 98%, or 99% identical to one of SEQ ID NOs: 331-797, 338-797 or 338-789. In some embodiments, the at least one SOCS1-targeting gRNA molecule is encoded by a DNA sequence that is 100% identical to one of SEQ ID NOs: 23-200, 56-200 or 56-187 and the at least one ZC3H12A-targeting gRNA molecule is encoded by a DNA sequence that is 100% identical to one of SEQ ID NOs: 331-797, 338-797 or 338-789.

In some embodiments, the gene-regulating system comprises at least two gRNA molecules, wherein at least one gRNA molecule is a PTPN2-targeting gRNA molecule and at least one gRNA molecule is a ZC3H12A-targeting gRNA molecule. In some embodiments, the at least one PTPN2-targeting gRNA molecule binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4) and the at least one ZC3H12A-targeting gRNA molecule binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one PTPN2-targeting gRNA molecule binds to a target DNA sequence that is 100% identical to a DNA sequence in the PTPN2 gene (SEQ ID NO: 3) or the Ptpn2 gene (SEQ ID NO: 4) and the at least one ZC3H12A-targeting gRNA molecule binds to a target DNA sequence that is 100% identical to a DNA sequence in the ZC3H12A gene (SEQ ID NO: 5) or the Zc3h12a gene (SEQ ID NO: 6).

In some embodiments, the at least one PTPN2-targeting gRNA molecule binds to a target DNA sequence that is at least 95%, 96%, 97%, 98%, or 99% identical to a DNA sequence defined by a set of genomic coordinates shown in Table 9 or Table 10 and the at least one ZC3H12A-targeting gRNA molecule binds to a target DNA sequence that is at least 95%, 96%, 97%, 98%, or 99% identical to a DNA sequence defined by a set of genomic coordinates shown in Table 11 or Table 12. In some embodiments, the at least one PTPN2-targeting gRNA molecule binds to a target DNA sequence that is 100% identical to a DNA sequence encoded by a DNA sequence defined by a set of genomic coordinates shown in Table 9 or Table 10 and the at least one ZC3H12A-targeting gRNA molecule binds to a target DNA sequence that is 100% identical to a DNA sequence encoded by a DNA sequence defined by a set of genomic coordinates shown in Table 11 or Table 12.

In some embodiments, the at least one PTPN2-targeting gRNA molecule binds to a target DNA sequence that is at least 95%, 96%, 97%, 98%, or 99% identical to one of SEQ ID NOs: 201-327 or 201-314 and the at least one ZC3H12A-targeting gRNA molecule binds to a target DNA sequence that is at least 95%, 96%, 97%, 98%, or 99% identical to one of SEQ ID NOs: 331-797, 338-797 or 338-789. In some embodiments, the at least one PTPN2-targeting gRNA molecule binds to a target DNA sequence that is 100% identical to one of SEQ ID NOs: 201-327 or 201-314 and the at least one ZC3H12A-targeting gRNA molecule binds to a target DNA sequence that is 100% identical to one of SEQ ID NOs: 331-797, 338-797 or 338-789.

In some embodiments, the at least one PTPN2-targeting gRNA molecule is encoded by a DNA sequence that is at least 95%, 96%, 97%, 98%, or 99% identical to one of SEQ ID NOs: 201-327 or 201-314 and the at least one ZC3H12A-targeting gRNA molecule is encoded by a DNA sequence that is at least 95%, 96%, 97%, 98%, or 99% identical to one of SEQ ID NOs: 331-797, 338-797 or 338-789. In some embodiments, the at least one PTPN2-targeting gRNA molecule is encoded by a DNA sequence that is 100% identical to one of SEQ ID NOs: 201-327 or 201-314 and the at least one ZC3H12A-targeting gRNA molecule is encoded by a DNA sequence that is 100% identical to one of SEQ ID NOs: 331-797, 338-797 or 338-789.

In some embodiments, the gene-regulating system comprises at least two gRNA molecules, wherein at least one gRNA molecule is a CBLB-targeting gRNA molecule and at least one gRNA molecule is a PTPN2-targeting gRNA molecule. In some embodiments, the at least one CBLB-targeting gRNA molecule binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one PTPN2-targeting gRNA molecule binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one CBLB-targeting gRNA molecule binds to a target DNA sequence that is 100% identical to a DNA sequence in the CBLB gene (SEQ ID NO: 7) or the Cblb gene (SEQ ID NO: 8) and the at least one PTPN2-targeting gRNA molecule binds to a target DNA sequence that is 100% identical to a DNA sequence in the PTPN2 gene (SEQ ID NO: 3) or the Ptpn2 gene (SEQ ID NO: 4).

In some embodiments, the at least one CBLB-targeting gRNA molecule binds to a target DNA sequence that is at least 95%, 96%, 97%, 98%, or 99% identical to a DNA sequence defined by a set of genomic coordinates shown in Table 17 or Table 18 and the at least one PTPN2-targeting gRNA molecule binds to a target DNA sequence that is at least 95%, 96%, 97%, 98%, or 99% identical to a DNA sequence defined by a set of genomic coordinates shown in Table 9 or Table 10. In some embodiments, the at least one CBLB-targeting gRNA molecule binds to a target DNA sequence that is 100% identical to a DNA sequence encoded by a DNA sequence defined by a set of genomic coordinates shown in Table 17 or Table 18 and the at least one PTPN2-targeting gRNA molecule binds to a target DNA sequence that is 100% identical to a DNA sequence encoded by a DNA sequence defined by a set of genomic coordinates shown in Table 9 or Table 10.

In some embodiments, the at least one CBLB-targeting gRNA molecule binds to a target DNA sequence that is at least 95%, 96%, 97%, 98%, or 99% identical to one of SEQ ID NOs: 798-823 or 798-808 and the at least one PTPN2-targeting gRNA molecule binds to a target DNA sequence that is at least 95%, 96%, 97%, 98%, or 99% identical to one of SEQ ID NOs: 201-327 or 201-314. In some embodiments, the at least one CBLB-targeting gRNA molecule binds to a target DNA sequence that is 100% identical to one of SEQ ID NOs: 798-823 or 798-808 and the at least one PTPN2-targeting gRNA molecule binds to a target DNA sequence that is 100% identical to one of SEQ ID NOs: 201-327 or 201-314.

In some embodiments, the at least one CBLB-targeting gRNA molecule is encoded by a DNA sequence that is at least 95%, 96%, 97%, 98%, or 99% identical to one of SEQ ID NOs: 798-823 or 798-808 and the at least one PTPN2-targeting gRNA molecule is encoded by a DNA sequence that is at least 95%, 96%, 97%, 98%, or 99% identical to one of SEQ ID NOs: 201-327 or 201-314. In some embodiments, the at least one CBLB-targeting gRNA molecule is encoded by a DNA sequence that is 100% identical to one of SEQ ID NOs: 798-823 or 798-808 and the at least one PTPN2-targeting gRNA molecule is encoded by a DNA sequence that is 100% identical to one of SEQ ID NOs: 201-327 or 201-314.

In some embodiments, the gene-regulating system comprises at least two gRNA molecules, wherein at least one gRNA molecule is a CBLB-targeting gRNA molecule and at least one gRNA molecule is a ZC3H12A-targeting gRNA molecule. In some embodiments, the at least one CBLB-targeting gRNA molecule binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one ZC3H12A-targeting gRNA molecule binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one CBLB-targeting gRNA molecule binds to a target DNA sequence that is 100% identical to a DNA sequence in the CBLB gene (SEQ ID NO: 7) or the Cblb gene (SEQ ID NO: 8) and the at least one ZC3H12A-targeting gRNA molecule binds to a target DNA sequence that is 100% identical to a DNA sequence in the ZC3H12A gene (SEQ ID NO: 5) or the Zc3h12a gene (SEQ ID NO: 6).

In some embodiments, the at least one CBLB-targeting gRNA molecule binds to a target DNA sequence that is at least 95%, 96%, 97%, 98%, or 99% identical to a DNA sequence defined by a set of genomic coordinates shown in Table 17 or Table 18 and the at least one ZC3H12A-targeting gRNA molecule binds to a target DNA sequence that is at least 95%, 96%, 97%, 98%, or 99% identical to a DNA sequence defined by a set of genomic coordinates shown in Table 11 or Table 12. In some embodiments, the at least one CBLB-targeting gRNA molecule binds to a target DNA sequence that is 100% identical to a DNA sequence encoded by a DNA sequence defined by a set of genomic coordinates shown in Table 17 or Table 18 and the at least one ZC3H12A-targeting gRNA molecule binds to a target DNA sequence that is 100% identical to a DNA sequence encoded by a DNA sequence defined by a set of genomic coordinates shown in Table 11 or Table 12.

In some embodiments, the at least one CBLB-targeting gRNA molecule binds to a target DNA sequence that is at least 95%, 96%, 97%, 98%, or 99% identical to one of SEQ ID NOs: 798-823 or 798-808 and the at least one ZC3H12A-targeting gRNA molecule binds to a target DNA sequence that is at least 95%, 96%, 97%, 98%, or 99% identical to one of SEQ ID NOs: 331-797, 338-797 or 338-789. In some embodiments, the at least one CBLB-targeting gRNA molecule binds to a target DNA sequence that is 100% identical to one of SEQ ID NOs: 798-823 or 798-808 and the at least one ZC3H12A-targeting gRNA molecule binds to a target DNA sequence that is 100% identical to one of SEQ ID NOs: 331-797, 338-797 or 338-789.

In some embodiments, the at least one CBLB-targeting gRNA molecule is encoded by a DNA sequence that is at least 95%, 96%, 97%, 98%, or 99% identical to one of SEQ ID NOs: 798-823 or 798-808 and the at least one ZC3H12A-targeting gRNA molecule is encoded by a DNA sequence that is at least 95%, 96%, 97%, 98%, or 99% identical to one of SEQ ID NOs: 331-797, 338-797 or 338-789. In some embodiments, the at least one CBLB-targeting gRNA molecule is encoded by a DNA sequence that is 100% identical to one of SEQ ID NOs: 798-823 or 798-808 and the at least one ZC3H12A-targeting gRNA molecule is encoded by a DNA sequence that is 100% identical to one of SEQ ID NOs: 331-797, 338-797 or 338-789.

In some embodiments, the gene-regulating system comprises at least two gRNA molecules, wherein at least one gRNA molecule is a SOCS1-targeting gRNA molecule and at least one gRNA molecule is a CBLB-targeting gRNA molecule. In some embodiments, the at least one SOCS1-targeting gRNA molecule binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one CBLB-targeting gRNA molecule binds to a target DNA sequence that is at least 95%, 96%, 97%, 98%, or 99% identical to a DNA sequence in the CBLB gene (SEQ ID NO: 7) or the Cblb gene (SEQ ID NO: 8). In some embodiments, the at least one SOCS1-targeting gRNA molecule binds to a target DNA sequence that is 100% identical to a DNA sequence in the SOCS1 gene (SEQ ID NO: 1) or the Socs1 gene (SEQ ID NO: 2) and the at least one CBLB-targeting gRNA molecule binds to a target DNA sequence that is 100% identical to a DNA sequence in the CBLB gene (SEQ ID NO: 7) or the Cblb gene (SEQ ID NO: 8).

In some embodiments, the at least one SOCS1-targeting gRNA molecule binds to a target DNA sequence that is at least 95%, 96%, 97%, 98%, or 99% identical to a DNA sequence defined by a set of genomic coordinates shown in Table 4 or Table 5 and the at least one CBLB-targeting gRNA molecule binds to a target DNA sequence that is at least 95%, 96%, 97%, 98%, or 99% identical to a DNA sequence defined by a set of genomic coordinates shown in Table 17 or Table 18. In some embodiments, the at least one SOCS1-targeting gRNA molecule binds to a target DNA sequence that is 100% identical to a DNA sequence encoded by a DNA sequence defined by a set of genomic coordinates shown in Table 4 or Table 5 and the at least one CBLB-targeting gRNA molecule binds to a target DNA sequence that is 100% identical to a DNA sequence encoded by a DNA sequence defined by a set of genomic coordinates shown in Table 17 or Table 18.

In some embodiments, the at least one SOCS1-targeting gRNA molecule binds to a target DNA sequence that is at least 95%, 96%, 97%, 98%, or 99% identical to one of SEQ ID NOs: 23-200, 56-200 or 56-187 and the at least one CBLB-targeting gRNA molecule binds to a target DNA sequence that is at least 95%, 96%, 97%, 98%, or 99% identical to one of SEQ ID NOs: 798-823 or 798-808. In some embodiments, the at least one SOCS1-targeting gRNA molecule binds to a target DNA sequence that is 100% identical to one of SEQ ID NOs: 23-200, 56-200 or 56-187 and the at least one CBLB-targeting gRNA molecule binds to a target DNA sequence that is 100% identical to one of SEQ ID NOs: 798-823 or 798-808.

In some embodiments, the at least one SOCS1-targeting gRNA molecule is encoded by a DNA sequence that is at least 95%, 96%, 97%, 98%, or 99% identical to one of SEQ ID NOs: 23-200, 56-200 or 56-187 and the at least one CBLB-targeting gRNA molecule is encoded by a DNA sequence that is at least 95%, 96%, 97%, 98%, or 99% identical to one of SEQ ID NOs: 798-823 or 798-808. In some embodiments, the at least one SOCS1-targeting gRNA molecule is encoded by a DNA sequence that is 100% identical to one of SEQ ID NOs: 23-200, 56-200 or 56-187 and the at least one CBLB-targeting gRNA molecule is encoded by a DNA sequence that is 100% identical to one of SEQ ID NOs: 798-823 or 798-808.

In some embodiments, the gene-regulating system comprises at least two gRNA molecules, wherein at least one gRNA molecule is a RC3H1-targeting gRNA molecule and at least one gRNA molecule is a PTPN2-targeting gRNA molecule. In some embodiments, the at least one RC3H1-targeting gRNA molecule binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one PTPN2-targeting gRNA molecule binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one RC3H1-targeting gRNA molecule binds to a target DNA sequence that is 100% identical to a DNA sequence in the RC3H1 gene (SEQ ID NO: 9) or the Rc3h1 gene (SEQ ID NO: 10) and the at least one PTPN2-targeting gRNA molecule binds to a target DNA sequence that is 100% identical to a DNA sequence in the PTPN2 gene (SEQ ID NO: 3) or the Ptpn2 gene (SEQ ID NO: 4).

In some embodiments, the at least one RC3H1-targeting gRNA molecule binds to a target DNA sequence that is at least 95%, 96%, 97%, 98%, or 99% identical to a DNA sequence defined by a set of genomic coordinates shown in Table 19 or Table 20 and the at least one PTPN2-targeting gRNA molecule binds to a target DNA sequence that is at least 95%, 96%, 97%, 98%, or 99% identical to a DNA sequence defined by a set of genomic coordinates shown in Table 9 or Table 10. In some embodiments, the at least one RC3H1-targeting gRNA molecule binds to a target DNA sequence that is 100% identical to a DNA sequence encoded by a DNA sequence defined by a set of genomic coordinates shown in Table 19 or Table 20 and the at least one PTPN2-targeting gRNA molecule binds to a target DNA sequence that is 100% identical to a DNA sequence encoded by a DNA sequence defined by a set of genomic coordinates shown in Table 9 or Table 10.

In some embodiments, the at least one RC3H1-targeting gRNA molecule binds to a target DNA sequence that is at least 95%, 96%, 97%, 98%, or 99% identical to one of SEQ ID NOs: 824-844 or 824-844 or 824-836 and the at least one PTPN2-targeting gRNA molecule binds to a target DNA sequence that is at least 95%, 96%, 97%, 98%, or 99% identical to one of SEQ ID NOs: 201-327 or 201-314. In some embodiments, the at least one RC3H1-targeting gRNA molecule binds to a target DNA sequence that is 100% identical to one of SEQ ID NOs: 824-844 or 824-844 or 824-836 and the at least one PTPN2-targeting gRNA molecule binds to a target DNA sequence that is 100% identical to one of SEQ ID NOs: 201-327 or 201-314.

In some embodiments, the at least one RC3H1-targeting gRNA molecule is encoded by a DNA sequence that is at least 95%, 96%, 97%, 98%, or 99% identical to one of SEQ ID NOs: 824-844 or 824-844 or 824-836 and the at least one PTPN2-targeting gRNA molecule is encoded by a DNA sequence that is at least 95%, 96%, 97%, 98%, or 99% identical to one of SEQ ID NOs: 201-327 or 201-314. In some embodiments, the at least one RC3H1-targeting gRNA molecule is encoded by a DNA sequence that is 100% identical to one of SEQ ID NOs: 824-844 or 824-844 or 824-836 and the at least one PTPN2-targeting gRNA molecule is encoded by a DNA sequence that is 100% identical to one of SEQ ID NOs: 201-327 or 201-314.

In some embodiments, the gene-regulating system comprises at least two gRNA molecules, wherein at least one gRNA molecule is a RC3H1-targeting gRNA molecule and at least one gRNA molecule is a ZC3H12A-targeting gRNA molecule. In some embodiments, the at least one RC3H1-targeting gRNA molecule binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one ZC3H12A-targeting gRNA molecule binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one RC3H1-targeting gRNA molecule binds to a target DNA sequence that is 100% identical to a DNA sequence in the RC3H1 gene (SEQ ID NO: 9) or the Rc3h1 gene (SEQ ID NO: 10) and the at least one ZC3H12A-targeting gRNA molecule binds to a target DNA sequence that is 100% identical to a DNA sequence in the ZC3H12A gene (SEQ ID NO: 5) or the Zc3h12a gene (SEQ ID NO: 6).

In some embodiments, the at least one RC3H1-targeting gRNA molecule binds to a target DNA sequence that is at least 95%, 96%, 97%, 98%, or 99% identical to a DNA sequence defined by a set of genomic coordinates shown in Table 19 or Table 20 and the at least one ZC3H12A-targeting gRNA molecule binds to a target DNA sequence that is at least 95%, 96%, 97%, 98%, or 99% identical to a DNA sequence defined by a set of genomic coordinates shown in Table 11 or Table 12. In some embodiments, the at least one RC3H1-targeting gRNA molecule binds to a target DNA sequence that is 100% identical to a DNA sequence encoded by a DNA sequence defined by a set of genomic coordinates shown in Table 19 or Table 20 and the at least one ZC3H12A-targeting gRNA molecule binds to a target DNA sequence that is 100% identical to a DNA sequence encoded by a DNA sequence defined by a set of genomic coordinates shown in Table 11 or Table 12.

In some embodiments, the at least one RC3H1-targeting gRNA molecule binds to a target DNA sequence that is at least 95%, 96%, 97%, 98%, or 99% identical to one of SEQ ID NOs: 824-844 or 824-844 or 824-836 and the at least one ZC3H12A-targeting gRNA molecule binds to a target DNA sequence that is at least 95%, 96%, 97%, 98%, or 99% identical to one of SEQ ID NOs: 331-797, 338-797 or 338-789. In some embodiments, the at least one RC3H1-targeting gRNA molecule binds to a target DNA sequence that is 100% identical to one of SEQ ID NOs: 824-844 or 824-844 or 824-836 and the at least one ZC3H12A-targeting gRNA molecule binds to a target DNA sequence that is 100% identical to one of SEQ ID NOs: 331-797, 338-797 or 338-789.

In some embodiments, the at least one RC3H1-targeting gRNA molecule is encoded by a DNA sequence that is at least 95%, 96%, 97%, 98%, or 99% identical to one of SEQ ID NOs: 824-844 or 824-844 or 824-836 and the at least one ZC3H12A-targeting gRNA molecule is encoded by a DNA sequence that is at least 95%, 96%, 97%, 98%, or 99% identical to one of SEQ ID NOs: 331-797, 338-797 or 338-789. In some embodiments, the at least one RC3H1-targeting gRNA molecule is encoded by a DNA sequence that is 100% identical to one of SEQ ID NOs: 824-844 or 824-844 or 824-836 and the at least one ZC3H12A-targeting gRNA molecule is encoded by a DNA sequence that is 100% identical to one of SEQ ID NOs: 331-797, 338-797 or 338-789.

In some embodiments, the gene-regulating system comprises at least two gRNA molecules, wherein at least one gRNA molecule is a SOCS1-targeting gRNA molecule and at least one gRNA molecule is a RC3H1-targeting gRNA molecule. In some embodiments, the at least one SOCS1-targeting gRNA molecule binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one RC3H1-targeting gRNA molecule binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10). In some embodiments, the at least one SOCS1-targeting gRNA molecule binds to a target DNA sequence that is 100% identical to a DNA sequence in the SOCS1 gene (SEQ ID NO: 1) or the Socs1 gene (SEQ ID NO: 2) and the at least one RC3H1-targeting gRNA molecule binds to a target DNA sequence that is 100% identical to a DNA sequence in the RC3H1 gene (SEQ ID NO: 9) or the Rc3h1 gene (SEQ ID NO: 10).

In some embodiments, the at least one SOCS1-targeting gRNA molecule binds to a target DNA sequence that is at least 95%, 96%, 97%, 98%, or 99% identical to a DNA sequence defined by a set of genomic coordinates shown in Table 4 or Table 5 and the at least one RC3H1-targeting gRNA molecule binds to a target DNA sequence that is at least 95%, 96%, 97%, 98%, or 99% identical to a DNA sequence defined by a set of genomic coordinates shown in Table 19 or Table 20. In some embodiments, the at least one SOCS1-targeting gRNA molecule binds to a target DNA sequence that is 100% identical to a DNA sequence encoded by a DNA sequence defined by a set of genomic coordinates shown in Table 4 or Table 5 and the at least one RC3H1-targeting gRNA molecule binds to a target DNA sequence that is 100% identical to a DNA sequence encoded by a DNA sequence defined by a set of genomic coordinates shown in Table 19 or Table 20.

In some embodiments, the at least one SOCS1-targeting gRNA molecule binds to a target DNA sequence that is at least 95%, 96%, 97%, 98%, or 99% identical to one of SEQ ID NOs: 23-200, 56-200 or 56-187 and the at least one RC3H1-targeting gRNA molecule binds to a target DNA sequence that is at least 95%, 96%, 97%, 98%, or 99% identical to one of SEQ ID NOs: 824-844 or 824-844 or 824-836. In some embodiments, the at least one SOCS1-targeting gRNA molecule binds to a target DNA sequence that is 100% identical to one of SEQ ID NOs: 23-200, 56-200 or 56-187 and the at least one RC3H1-targeting gRNA molecule binds to a target DNA sequence that is 100% identical to one of SEQ ID NOs: 824-844 or 824-844 or 824-836.

In some embodiments, the at least one SOCS1-targeting gRNA molecule is encoded by a DNA sequence that is at least 95%, 96%, 97%, 98%, or 99% identical to one of SEQ ID NOs: 23-200, 56-200 or 56-187 and the at least one RC3H1-targeting gRNA molecule is encoded by a DNA sequence that is at least 95%, 96%, 97%, 98%, or 99% identical to one of SEQ ID NOs: 824-844 or 824-844 or 824-836. In some embodiments, the at least one SOCS1-targeting gRNA molecule is encoded by a DNA sequence that is 100% identical to one of SEQ ID NOs: 23-200, 56-200 or 56-187 and the at least one RC3H1-targeting gRNA molecule is encoded by a DNA sequence that is 100% identical to one of SEQ ID NOs: 824-844 or 824-844 or 824-836.

In some embodiments, the gene-regulating system comprises at least two gRNA molecules, wherein at least one gRNA molecule is a CBLB-targeting gRNA molecule and at least one gRNA molecule is a RC3H1-targeting gRNA molecule. In some embodiments, the at least one CBLB-targeting gRNA molecule binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one RC3H1-targeting gRNA molecule binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10). In some embodiments, the at least one CBLB-targeting gRNA molecule binds to a target DNA sequence that is 100% identical to a DNA sequence in the CBLB gene (SEQ ID NO: 7) or the Cblb gene (SEQ ID NO: 8) and the at least one RC3H1-targeting gRNA molecule binds to a target DNA sequence that is 100% identical to a DNA sequence in the RC3H1 gene (SEQ ID NO: 9) or the Rc3h1 gene (SEQ ID NO: 10).

In some embodiments, the at least one CBLB-targeting gRNA molecule binds to a target DNA sequence that is at least 95%, 96%, 97%, 98%, or 99% identical to a DNA sequence defined by a set of genomic coordinates shown in Table 17 or Table 18 and the at least one RC3H1-targeting gRNA molecule binds to a target DNA sequence that is at least 95%, 96%, 97%, 98%, or 99% identical to a DNA sequence defined by a set of genomic coordinates shown in Table 19 or Table 20. In some embodiments, the at least one CBLB-targeting gRNA molecule binds to a target DNA sequence that is 100% identical to a DNA sequence encoded by a DNA sequence defined by a set of genomic coordinates shown in Table 17 or Table 18 and the at least one RC3H1-targeting gRNA molecule binds to a target DNA sequence that is 100% identical to a DNA sequence encoded by a DNA sequence defined by a set of genomic coordinates shown in Table 19 or Table 20.

In some embodiments, the at least one CBLB-targeting gRNA molecule binds to a target DNA sequence that is at least 95%, 96%, 97%, 98%, or 99% identical to one of SEQ ID NOs: 798-823 or 798-808 and the at least one RC3H1-targeting gRNA molecule binds to a target DNA sequence that is at least 95%, 96%, 97%, 98%, or 99% identical to one of SEQ ID NOs: 824-844 or 824-844 or 824-836. In some embodiments, the at least one CBLB-targeting gRNA molecule binds to a target DNA sequence that is 100% identical to one of SEQ ID NOs: 798-823 or 798-808 and the at least one RC3H1-targeting gRNA molecule binds to a target DNA sequence that is 100% identical to one of SEQ ID NOs: 824-844 or 824-844 or 824-836.

In some embodiments, the at least one CBLB-targeting gRNA molecule is encoded by a DNA sequence that is at least 95%, 96%, 97%, 98%, or 99% identical to one of SEQ ID NOs: 798-823 or 798-808 and the at least one RC3H1-targeting gRNA molecule is encoded by a DNA sequence that is at least 95%, 96%, 97%, 98%, or 99% identical to one of SEQ ID NOs: 824-844 or 824-844 or 824-836. In some embodiments, the at least one CBLB-targeting gRNA molecule is encoded by a DNA sequence that is 100% identical to one of SEQ ID NOs: 798-823 or 798-808 and the at least one RC3H1-targeting gRNA molecule is encoded by a DNA sequence that is 100% identical to one of SEQ ID NOs: 824-844 or 824-844 or 824-836.

In some embodiments, the gene-regulating system comprises at least two gRNA molecules, wherein at least one gRNA molecule is a NFKBIA-targeting gRNA molecule and at least one gRNA molecule is a PTPN2-targeting gRNA molecule. In some embodiments, the at least one NFKBIA-targeting gRNA molecule binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12) and the at least one PTPN2-targeting gRNA molecule binds to a target DNA sequence that is at least 95%, 96%, 97%, 98%, or 99% identical to a DNA sequence in the PTPN2 gene (SEQ ID NO: 3) or the Ptpn2 gene (SEQ ID NO: 4). In some embodiments, the at least one NFKBIA-targeting gRNA molecule binds to a target DNA sequence that is 100% identical to a DNA sequence in the NFKBIA gene (SEQ ID NO: 11) or the Nfkbia gene (SEQ ID NO: 12) and the at least one PTPN2-targeting gRNA molecule binds to a target DNA sequence that is 100% identical to a DNA sequence in the PTPN2 gene (SEQ ID NO: 3) or the Ptpn2 gene (SEQ ID NO: 4).

In some embodiments, the at least one NFKBIA-targeting gRNA molecule binds to a target DNA sequence that is at least 95%, 96%, 97%, 98%, or 99% identical to a DNA sequence defined by a set of genomic coordinates shown in Table 21 or Table 22 and the at least one PTPN2-targeting gRNA molecule binds to a target DNA sequence that is at least 95%, 96%, 97%, 98%, or 99% identical to a DNA sequence defined by a set of genomic coordinates shown in Table 9 or Table 10. In some embodiments, the at least one NFKBIA-targeting gRNA molecule binds to a target DNA sequence that is 100% identical to a DNA sequence encoded by a DNA sequence defined by a set of genomic coordinates shown in Table 21 or Table 22 and the at least one PTPN2-targeting gRNA molecule binds to a target DNA sequence that is 100% identical to a DNA sequence encoded by a DNA sequence defined by a set of genomic coordinates shown in Table 9 or Table 10.

In some embodiments, the at least one NFKBIA-targeting gRNA molecule binds to a target DNA sequence that is at least 95%, 96%, 97%, 98%, or 99% identical to one of SEQ ID NOs: 845-875 or 845-856 and the at least one PTPN2-targeting gRNA molecule binds to a target DNA sequence that is at least 95%, 96%, 97%, 98%, or 99% identical to one of SEQ ID NOs: 201-327 or 201-314. In some embodiments, the at least one NFKBIA-targeting gRNA molecule binds to a target DNA sequence that is 100% identical to one of SEQ ID NOs: 845-875 or 845-856 and the at least one PTPN2-targeting gRNA molecule binds to a target DNA sequence that is 100% identical to one of SEQ ID NOs: 201-327 or 201-314.

In some embodiments, the at least one NFKBIA-targeting gRNA molecule is encoded by a DNA sequence that is at least 95%, 96%, 97%, 98%, or 99% identical to one of SEQ ID NOs: 845-875 or 845-856 and the at least one PTPN2-targeting gRNA molecule is encoded by a DNA sequence that is at least 95%, 96%, 97%, 98%, or 99% identical to one of SEQ ID NOs: 201-327 or 201-314. In some embodiments, the at least one NFKBIA-targeting gRNA molecule is encoded by a DNA sequence that is 100% identical to one of SEQ ID NOs: 845-875 or 845-856 and the at least one PTPN2-targeting gRNA molecule is encoded by a DNA sequence that is 100% identical to one of SEQ ID NOs: 201-327 or 201-314.

In some embodiments, the gene-regulating system comprises at least two gRNA molecules, wherein at least one gRNA molecule is a NFKBIA-targeting gRNA molecule and at least one gRNA molecule is a ZC3H12A-targeting gRNA molecule. In some embodiments, the at least one NFKBIA-targeting gRNA molecule binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12) and the at least one ZC3H12A-targeting gRNA molecule binds to a target DNA sequence that is at least 95%, 96%, 97%, 98%, or 99% identical to a DNA sequence in the ZC3H12A gene (SEQ ID NO: 5) or the Zc3h12a gene (SEQ ID NO: 6). In some embodiments, the at least one NFKBIA-targeting gRNA molecule binds to a target DNA sequence that is 100% identical to a DNA sequence in the NFKBIA gene (SEQ ID NO: 11) or the Nfkbia gene (SEQ ID NO: 12) and the at least one ZC3H12A-targeting gRNA molecule binds to a target DNA sequence that is 100% identical to a DNA sequence in the ZC3H12A gene (SEQ ID NO: 5) or the Zc3h12a gene (SEQ ID NO: 6).

In some embodiments, the at least one NFKBIA-targeting gRNA molecule binds to a target DNA sequence that is at least 95%, 96%, 97%, 98%, or 99% identical to a DNA sequence defined by a set of genomic coordinates shown in Table 21 or Table 22 and the at least one ZC3H12A-targeting gRNA molecule binds to a target DNA sequence that is at least 95%, 96%, 97%, 98%, or 99% identical to a DNA sequence defined by a set of genomic coordinates shown in Table 11 or Table 12. In some embodiments, the at least one NFKBIA-targeting gRNA molecule binds to a target DNA sequence that is 100% identical to a DNA sequence encoded by a DNA sequence defined by a set of genomic coordinates shown in Table 21 or Table 22 and the at least one ZC3H12A-targeting gRNA molecule binds to a target DNA sequence that is 100% identical to a DNA sequence encoded by a DNA sequence defined by a set of genomic coordinates shown in Table 11 or Table 12.

In some embodiments, the at least one NFKBIA-targeting gRNA molecule binds to a target DNA sequence that is at least 95%, 96%, 97%, 98%, or 99% identical to one of SEQ ID NOs: 845-875 or 845-856 and the at least one ZC3H12A-targeting gRNA molecule binds to a target DNA sequence that is at least 95%, 96%, 97%, 98%, or 99% identical to one of SEQ ID NOs: 331-797, 338-797 or 338-789. In some embodiments, the at least one NFKBIA-targeting gRNA molecule binds to a target DNA sequence that is 100% identical to one of SEQ ID NOs: 845-875 or 845-856 and the at least one ZC3H12A-targeting gRNA molecule binds to a target DNA sequence that is 100% identical to one of SEQ ID NOs: 331-797, 338-797 or 338-789.

In some embodiments, the at least one NFKBIA-targeting gRNA molecule is encoded by a DNA sequence that is at least 95%, 96%, 97%, 98%, or 99% identical to one of SEQ ID NOs: 845-875 or 845-856 and the at least one ZC3H12A-targeting gRNA molecule is encoded by a DNA sequence that is at least 95%, 96%, 97%, 98%, or 99% identical to one of SEQ ID NOs: 331-797, 338-797 or 338-789. In some embodiments, the at least one NFKBIA-targeting gRNA molecule is encoded by a DNA sequence that is 100% identical to one of SEQ ID NOs: 845-875 or 845-856 and the at least one ZC3H12A-targeting gRNA molecule is encoded by a DNA sequence that is 100% identical to one of SEQ ID NOs: 331-797, 338-797 or 338-789.

In some embodiments, the gene-regulating system comprises at least two gRNA molecules, wherein at least one gRNA molecule is a SOCS1-targeting gRNA molecule and at least one gRNA molecule is a NFKBIA-targeting gRNA molecule. In some embodiments, the at least one SOCS1-targeting gRNA molecule binds to a target DNA sequence that is at least 95%, 96%, 97%, 98%, or 99% identical to a DNA sequence in the SOCS1 gene (SEQ ID NO: 1) or the Socs1 gene (SEQ ID NO: 2) and the at least one NFKBIA-targeting gRNA molecule binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one SOCS1-targeting gRNA molecule binds to a target DNA sequence that is 100% identical to a DNA sequence in the SOCS1 gene (SEQ ID NO: 1) or the Socs1 gene (SEQ ID NO: 2) and the at least one NFKBIA-targeting gRNA molecule binds to a target DNA sequence that is 100% identical to a DNA sequence in the NFKBIA gene (SEQ ID NO: 11) or the Nfkbia gene (SEQ ID NO: 12).

In some embodiments, the at least one SOCS1-targeting gRNA molecule binds to a target DNA sequence that is at least 95%, 96%, 97%, 98%, or 99% identical to a DNA sequence defined by a set of genomic coordinates shown in Table 4 or Table 5 and the at least one NFKBIA-targeting gRNA molecule binds to a target DNA sequence that is at least 95%, 96%, 97%, 98%, or 99% identical to a DNA sequence defined by a set of genomic coordinates shown in Table 21 or Table 22. In some embodiments, the at least one SOCS1-targeting gRNA molecule binds to a target DNA sequence that is 100% identical to a DNA sequence encoded by a DNA sequence defined by a set of genomic coordinates shown in Table 4 or Table 5 and the at least one NFKBIA-targeting gRNA molecule binds to a target DNA sequence that is 100% identical to a DNA sequence encoded by a DNA sequence defined by a set of genomic coordinates shown in Table 21 or Table 22.

In some embodiments, the at least one SOCS1-targeting gRNA molecule binds to a target DNA sequence that is at least 95%, 96%, 97%, 98%, or 99% identical to one of SEQ ID NOs: 23-200, 56-200 or 56-187 and the at least one NFKBIA-targeting gRNA molecule binds to a target DNA sequence that is at least 95%, 96%, 97%, 98%, or 99% identical to one of SEQ ID NOs: 845-875 or 845-856. In some embodiments, the at least one SOCS1-targeting gRNA molecule binds to a target DNA sequence that is 100% identical to one of SEQ ID NOs: 23-200, 56-200 or 56-187 and the at least one NFKBIA-targeting gRNA molecule binds to a target DNA sequence that is 100% identical to one of SEQ ID NOs: 845-875 or 845-856.

In some embodiments, the at least one SOCS1-targeting gRNA molecule is encoded by a DNA sequence that is at least 95%, 96%, 97%, 98%, or 99% identical to one of SEQ ID NOs: 23-200, 56-200 or 56-187 and the at least one NFKBIA-targeting gRNA molecule is encoded by a DNA sequence that is at least 95%, 96%, 97%, 98%, or 99% identical to one of SEQ ID NOs: 845-875 or 845-856. In some embodiments, the at least one SOCS1-targeting gRNA molecule is encoded by a DNA sequence that is 100% identical to one of SEQ ID NOs: 23-200, 56-200 or 56-187 and the at least one NFKBIA-targeting gRNA molecule is encoded by a DNA sequence that is 100% identical to one of SEQ ID NOs: 845-875 or 845-856.

In some embodiments, the gene-regulating system comprises at least two gRNA molecules, wherein at least one gRNA molecule is a CBLB-targeting gRNA molecule and at least one gRNA molecule is a NFKBIA-targeting gRNA molecule. In some embodiments, the at least one CBLB-targeting gRNA molecule binds to a target DNA sequence that is at least 95%, 96%, 97%, 98%, or 99% identical to a DNA sequence in the CBLB gene (SEQ ID NO: 7) or the Cblb gene (SEQ ID NO: 8) and the at least one NFKBIA-targeting gRNA molecule binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one CBLB-targeting gRNA molecule binds to a target DNA sequence that is 100% identical to a DNA sequence in the CBLB gene (SEQ ID NO: 7) or the Cblb gene (SEQ ID NO: 8) and the at least one NFKBIA-targeting gRNA molecule binds to a target DNA sequence that is 100% identical to a DNA sequence in the NFKBIA gene (SEQ ID NO: 11) or the Nfkbia gene (SEQ ID NO: 12).

In some embodiments, the at least one CBLB-targeting gRNA molecule binds to a target DNA sequence that is at least 95%, 96%, 97%, 98%, or 99% identical to a DNA sequence defined by a set of genomic coordinates shown in Table 17 or Table 18 and the at least one NFKBIA-targeting gRNA molecule binds to a target DNA sequence that is at least 95%, 96%, 97%, 98%, or 99% identical to a DNA sequence defined by a set of genomic coordinates shown in Table 21 or Table 22. In some embodiments, the at least one CBLB-targeting gRNA molecule binds to a target DNA sequence that is 100% identical to a DNA sequence encoded by a DNA sequence defined by a set of genomic coordinates shown in Table 17 or Table 18 and the at least one NFKBIA-targeting gRNA molecule binds to a target DNA sequence that is 100% identical to a DNA sequence encoded by a DNA sequence defined by a set of genomic coordinates shown in Table 21 or Table 22.

In some embodiments, the at least one CBLB-targeting gRNA molecule binds to a target DNA sequence that is at least 95%, 96%, 97%, 98%, or 99% identical to one of SEQ ID NOs: 798-823 or 798-808 and the at least one NFKBIA-targeting gRNA molecule binds to a target DNA sequence that is at least 95%, 96%, 97%, 98%, or 99% identical to one of SEQ ID NOs: 845-875 or 845-856. In some embodiments, the at least one CBLB-targeting gRNA molecule binds to a target DNA sequence that is 100% identical to one of SEQ ID NOs: 798-823 or 798-808 and the at least one NFKBIA-targeting gRNA molecule binds to a target DNA sequence that is 100% identical to one of SEQ ID NOs: 845-875 or 845-856.

In some embodiments, the at least one CBLB-targeting gRNA molecule is encoded by a DNA sequence that is at least 95%, 96%, 97%, 98%, or 99% identical to one of SEQ ID NOs: 798-823 or 798-808 and the at least one NFKBIA-targeting gRNA molecule is encoded by a DNA sequence that is at least 95%, 96%, 97%, 98%, or 99% identical to one of SEQ ID NOs: 845-875 or 845-856. In some embodiments, the at least one CBLB-targeting gRNA molecule is encoded by a DNA sequence that is 100% identical to one of SEQ ID NOs: 798-823 or 798-808 and the at least one NFKBIA-targeting gRNA molecule is encoded by a DNA sequence that is 100% identical to one of SEQ ID NOs: 845-875 or 845-856.

In some embodiments, the gene-regulating system comprises at least two gRNA molecules, wherein at least one gRNA molecule is a RC3H1-targeting gRNA molecule and at least one gRNA molecule is a NFKBIA-targeting gRNA molecule. In some embodiments, the at least one RC3H1-targeting gRNA molecule binds to a target DNA sequence that is at least 95%, 96%, 97%, 98%, or 99% identical to a DNA sequence in the RC3H1 gene (SEQ ID NO: 9) or the Rc3h1 gene (SEQ ID NO: 10) and the at least one NFKBIA-targeting gRNA molecule binds to a target DNA sequence that is at least 95%, 96%, 97%, 98%, or 99% identical to a DNA sequence in the NFKBIA gene (SEQ ID NO: 11) or the Nfkbia gene (SEQ ID NO: 12). In some embodiments, the at least one RC3H1-targeting gRNA molecule binds to a target DNA sequence that is 100% identical to a DNA sequence in the RC3H1 gene (SEQ ID NO: 9) or the Rc3h1 gene (SEQ ID NO: 10) and the at least one NFKBIA-targeting gRNA molecule binds to a target DNA sequence that is 100% identical to a DNA sequence in the NFKBIA gene (SEQ ID NO: 11) or the Nfkbia gene (SEQ ID NO: 12).

In some embodiments, the at least one RC3H1-targeting gRNA molecule binds to a target DNA sequence that is at least 95%, 96%, 97%, 98%, or 99% identical to a DNA sequence defined by a set of genomic coordinates shown in Table 19 or Table 20 and the at least one NFKBIA-targeting gRNA molecule binds to a target DNA sequence that is at least 95%, 96%, 97%, 98%, or 99% identical to a DNA sequence defined by a set of genomic coordinates shown in Table 21 or Table 22. In some embodiments, the at least one RC3H1-targeting gRNA molecule binds to a target DNA sequence that is 100% identical to a DNA sequence encoded by a DNA sequence defined by a set of genomic coordinates shown in Table 19 or Table 20 and the at least one NFKBIA-targeting gRNA molecule binds to a target DNA sequence that is 100% identical to a DNA sequence encoded by a DNA sequence defined by a set of genomic coordinates shown in Table 21 or Table 22.

In some embodiments, the at least one RC3H1-targeting gRNA molecule binds to a target DNA sequence that is at least 95%, 96%, 97%, 98%, or 99% identical to one of SEQ ID NOs: 824-844 or 824-844 or 824-836 and the at least one NFKBIA-targeting gRNA molecule binds to a target DNA sequence that is at least 95%, 96%, 97%, 98%, or 99% identical to one of SEQ ID NOs: 845-875 or 845-856. In some embodiments, the at least one RC3H1-targeting gRNA molecule binds to a target DNA sequence that is 100% identical to one of SEQ ID NOs: 824-844 or 824-844 or 824-836 and the at least one NFKBIA-targeting gRNA molecule binds to a target DNA sequence that is 100% identical to one of SEQ ID NOs: 845-875 or 845-856.

In some embodiments, the at least one RC3H1-targeting gRNA molecule is encoded by a DNA sequence that is at least 95%, 96%, 97%, 98%, or 99% identical to one of SEQ ID NOs: 824-844 or 824-844 or 824-836 and the at least one NFKBIA-targeting gRNA molecule is encoded by a DNA sequence that is at least 95%, 96%, 97%, 98%, or 99% identical to one of SEQ ID NOs: 845-875 or 845-856. In some embodiments, the at least one RC3H1-targeting gRNA molecule is encoded by a DNA sequence that is 100% identical to one of SEQ ID NOs: 824-844 or 824-844 or 824-836 and the at least one NFKBIA-targeting gRNA molecule is encoded by a DNA sequence that is 100% identical to one of SEQ ID NOs: 845-875 or 845-856.

In some embodiments, the nucleic acid-binding segments of the gRNA sequences described herein are designed to minimize off-target binding using algorithms known in the art (e.g., Cas-OFF finder) to identify target sequences that are unique to a particular target locus or target gene.

In some embodiments, the gRNAs described herein can comprise one or more modified nucleosides or nucleotides which introduce stability toward nucleases. In such embodiments, these modified gRNAs may elicit a reduced innate immune as compared to a non-modified gRNA. The term “innate immune response” includes a cellular response to exogenous nucleic acids, including single stranded nucleic acids, generally of viral or bacterial origin, which involves the induction of cytokine expression and release, particularly the interferons, and cell death.

In some embodiments, the gRNAs described herein are modified at or near the 5′ end (e.g., within 1-10, 1-5, or 1-2 nucleotides of their 5′ end). In some embodiments, the 5′ end of a gRNA is modified by the inclusion of a eukaryotic mRNA cap structure or cap analog (e.g., a G(5′)ppp(5′)G cap analog, a m7G(5′)ppp(5′)G cap analog, or a 3′-0-Me-m7G(5′)ppp(5′)G anti reverse cap analog (ARCA)). In some embodiments, an in vitro transcribed gRNA is modified by treatment with a phosphatase (e.g., calf intestinal alkaline phosphatase) to remove the 5′ triphosphate group. In some embodiments, a gRNA comprises a modification at or near its 3′ end (e.g., within 1-10, 1-5, or 1-2 nucleotides of its 3′ end). For example, in some embodiments, the 3′ end of a gRNA is modified by the addition of one or more (e.g., 25-200) adenine (A) residues.

In some embodiments, modified nucleosides and modified nucleotides can be present in a gRNA, but also may be present in other gene-regulating systems, e.g., mRNA, RNAi, or siRNA-based systems. In some embodiments, modified nucleosides and nucleotides can include one or more of:
a. alteration, e.g., replacement, of one or both of the non-linking phosphate oxygens and/or of one or more of the linking phosphate oxygens in the phosphodiester backbone linkage;
b. alteration, e.g., replacement, of a constituent of the ribose sugar, e.g., of the 2′ hydroxyl on the ribose sugar;
c. wholesale replacement of the phosphate moiety with “dephospho” linkers;
d. modification or replacement of a naturally occurring nucleobase;
e. replacement or modification of the ribose-phosphate backbone;
f. modification of the 3′ end or 5′ end of the oligonucleotide, e.g., removal, modification or replacement of a terminal phosphate group or conjugation of a moiety; and
g. modification of the sugar.

In some embodiments, the modifications listed above can be combined to provide modified nucleosides and nucleotides that can have two, three, four, or more modifications. For example, in some embodiments, a modified nucleoside or nucleotide can have a modified sugar and a modified nucleobase. In some embodiments, every base of a gRNA is modified. In some embodiments, each of the phosphate groups of a gRNA molecule are replaced with phosphorothioate groups.

In some embodiments, a software tool can be used to optimize the choice of gRNA within a user's target sequence, e.g., to minimize total off-target activity across the genome. Off target activity may be other than cleavage. For example, for each possible gRNA choice using S. pyogenes Cas9, software tools can identify all potential off-target sequences (preceding either NAG or NGG PAMs) across the genome that contain up to a certain number (e.g., 1, 2, 3, 4, 5, 6, 7, 8, 9, or 10) of mismatched base-pairs. The cleavage efficiency at each off-target sequence can be predicted, e.g., using an experimentally-derived weighting scheme. Each possible gRNA can then be ranked according to its total predicted off-target cleavage; the top-ranked gRNAs represent those that are likely to have the greatest on-target and the at least off-target cleavage. Other functions, e.g., automated reagent design for gRNA vector construction, primer design for the on-target Surveyor assay, and primer design for high-throughput detection and quantification of off-target cleavage via next-generation sequencing, can also be included in the tool.

### III. Methods of Producing Modified TILs

In some embodiments, the present disclosure provides improved methods for producing modified TILs. In some embodiments, the methods comprise introducing a gene-regulating system into a population of immune effector cells wherein the gene-regulating system is capable of reducing expression and/or function of one, two or more endogenous target genes selected from ANKRD11, BCL2L11, BCL3, BCOR, CALM2, CBLB, CHIC2, CTLA4, DHODH, E2F8, EGR2, FL11, FOXP3, GATA3, GNAS, HAVCR2, IKZF1, IKZF2, IKZF3, LAG3, MAP4K, NFKBIA, NR4A3, NRP1, PBRM1, PCBP1, PDCD1, PELI1, PIK3CD, PPP2R2D, PTPN1, PTPN2, PTPN22, PTPN6, RBM39, RC3H1, SEMA7A, SERPINA3, SETD5, SH2B3, SH2D1A, SMAD2, SOCS1, TANK, TGFBR1, TGFBR2, TIGIT, TNFAIP3, TNIP1, TRAF6, UMPS, WDR6 and ZC3H12A. (See International Publication Nos. WO 2019/178422, WO 2019/178420 and WO 2019/178421, incorporated by reference herein in their entireties.) In some embodiments, the one, two or more endogenous target genes selected from SOCS1, PTPN2, ZC3H12A, CBLB, RC3H1 and NFKBIA.

The components of the gene-regulating systems described herein, e.g., a nucleic acid-, protein-, or nucleic acid/protein-based system can be introduced into target cells in a variety of forms using a variety of delivery methods and formulations. In some embodiments, a polynucleotide encoding one or more components of the system is delivered by a recombinant vector (e.g., a viral vector or plasmid). In some embodiments, where the system comprises more than a single component, a vector may comprise a plurality of polynucleotides, each encoding a component of the system. In some embodiments, where the system comprises more than a single component, a plurality of vectors may be used, wherein each vector comprises a polynucleotide encoding a particular component of the system. In some embodiments, a vector may also comprise a sequence encoding a signal peptide (e.g., for nuclear localization, nucleolar localization, mitochondrial localization), fused to the polynucleotide encoding the one or more components of the system. For example, a vector may comprise a nuclear localization sequence (e.g., from SV40) fused to the polynucleotide encoding the one or more components of the system. In some embodiments, the introduction of the gene-regulating system to the cell occurs in vitro. In some embodiments, the introduction of the gene-regulating system to the cell occurs in vivo. In some embodiments, the introduction of the gene-regulating system to the cell occurs ex vivo.

In some embodiments, the recombinant vector comprising a polynucleotide encoding one or more components of a gene-regulating system described herein is a viral vector. Suitable viral vectors include, but are not limited to, viral vectors based on vaccinia virus; poliovirus; adenovirus (see, e.g., Li et al., Invest Opthalmol Vis Sci 35:2543 2549, 1994; Borras et al., Gene Ther 6:515 524, 1999; Li and Davidson, PNAS 92:7700 7704, 1995; Sakamoto et al., H Gene Ther 5:1088 1097, 1999; WO 94/12649, WO 93/03769; WO 93/19191; WO 94/28938; WO 95/11984 and WO 95/00655); adeno-associated virus (see, e.g., U.S. Pat. No. 7,078,387; Ali et al., Hum Gene Ther 9:81 86, 1998, Flannery et al,, PNAS 94:6916 6921, 1997; Bennett et al., Invest Opthalmol Vis Sci 38:2857 2863, 1997; Jomary et al., Gene Ther 4:683 690, 1997, Rolling et al., Hum Gene Ther 10:641 648, 1999; Ali et al., Hum Mol Genet 5:591 594, 1996; Srivastava in WO 93/09239, Samulski et al., J. Vir. (1989) 63:3822-3828; Mendelson et al,, Virol. (1988) 166:154-165; and Flotte et al., PNAS (1993) 90:10613-10617); SV40; herpes simplex virus; human immunodeficiency virus (see, e.g., Miyoshi et al., PNAS 94:10319 23, 1997; Takahashi et al., J Virol 73:7812 7816, 1999); a retroviral vector (e.g., Murine Leukemia Virus, spleen necrosis virus, and vectors derived from retroviruses such as Rous Sarcoma Virus, Harvey Sarcoma Virus, avian leukosis virus, a lentivirus, human immunodeficiency virus, myeloproliferative sarcoma virus, and mammary tumor virus); and the like.

In some embodiments, the recombinant vector comprising a polynucleotide encoding one or more components of a gene-regulating system described herein is a plasmid. Numerous suitable plasmid expression vectors are known to those of skill in the art, and many are commercially available. The following vectors are provided by way of example; for eukaryotic host cells: pXT1, pSG5 (Stratagene), pSVK3, pBPV, pMSG, and pSVLSV40 (Pharmacia). However, any other plasmid vector may be used so long as it is compatible with the host cell. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

In some embodiments, a polynucleotide sequence encoding one or more components of a gene-regulating system described herein is operably linked to a control element, e.g., a transcriptional control element, such as a promoter. The transcriptional control element may be functional in either a eukaryotic cell (e.g., a mammalian cell) or a prokaryotic cell (e.g., bacterial or archaeal cell). In some embodiments, a polynucleotide sequence encoding one or more components of a gene-regulating system described herein is operably linked to multiple control elements that allow expression of the polynucleotide in both prokaryotic and eukaryotic cells. Depending on the cell type and gene-regulating system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544).

Non-limiting examples of suitable eukaryotic promoters (promoters functional in a eukaryotic cell) include those from cytomegalovirus (CMV) immediate early, herpes simplex virus (HSV) thymidine kinase, early and late SV40, long terminal repeats (LTRs) from retrovirus, and mouse metallothionein-1. Selection of the appropriate vector and promoter is well within the level of ordinary skill in the art. The expression vector may also contain a ribosome binding site for translation initiation and a transcription terminator. The expression vector may also include appropriate sequences for amplifying expression. The expression vector may also include nucleotide sequences encoding protein tags (e.g., 6xHis tag, hemagglutinin tag, green fluorescent protein, etc.) that are fused to the site-directed modifying polypeptide, thus resulting in a chimeric polypeptide.

In some embodiments, a polynucleotide sequence encoding one or more components of a gene-regulating system described herein is operably linked to an inducible promoter. In some embodiments, a polynucleotide sequence encoding one or more components of a gene-regulating system described herein is operably linked to a constitutive promoter.

Methods of introducing polynucleotides and recombinant vectors into a host cell are known in the art, and any known method can be used to introduce components of a gene-regulating system into a cell. Suitable methods include e.g., viral or bacteriophage infection, transfection, conjugation, protoplast fusion, lipofection, electroporation, calcium phosphate precipitation, polyethyleneimine (PEI)-mediated transfection, DEAE-dextran mediated transfection, liposome-mediated transfection, particle gun technology, calcium phosphate precipitation, direct micro injection, nanoparticle-mediated nucleic acid delivery (see, e.g., Panyam et al., Adv Drug Deliv Rev. 2012 Sep. 13. pii: 50169-409X(12)00283-9), microfluidics delivery methods (See e.g., International PCT Publication No. WO 2013/059343), and the like. In some embodiments, delivery via electroporation comprises mixing the cells with the components of a gene-regulating system in a cartridge, chamber, or cuvette and applying one or more electrical impulses of defined duration and amplitude. In some embodiments, cells are mixed with components of a gene-regulating system in a vessel connected to a device (e.g., a pump) which feeds the mixture into a cartridge, chamber, or cuvette wherein one or more electrical impulses of defined duration and amplitude are applied, after which the cells are delivered to a second vessel.

In some embodiments, electroporation is used to introduce components of a gene-regulating system into a cell. In some embodiments where a pre-REP and REP protocol is used, electroporation is used to introduce components of a gene-regulating system into a cell after the pre-REP stage but before the REP stage.

In some embodiments, one or more components of a gene-regulating system, or polynucleotide sequence encoding one or more components of a gene-regulating system described herein are introduced to a cell in a non-viral delivery vehicle, such as a transposon, a nanoparticle (e.g., a lipid nanoparticle), a liposome, an exosome, an attenuated bacterium, or a virus-like particle. In some embodiments, the vehicle is an attenuated bacterium (e.g., naturally or artificially engineered to be invasive but attenuated to prevent pathogenesis including Listeria monocytogenes, certain Salmonella strains, Bifidobacterium longum, and modified Escherichia coli), bacteria having nutritional and tissue-specific tropism to target specific cells, and bacteria having modified surface proteins to alter target cell specificity. In some embodiments, the vehicle is a genetically modified bacteriophage (e.g., engineered phages having large packaging capacity, less immunogenicity, containing mammalian plasmid maintenance sequences and having incorporated targeting ligands). In some embodiments, the vehicle is a mammalian virus-like particle. For example, modified viral particles can be generated (e.g., by purification of the “empty” particles followed by ex vivo assembly of the virus with the desired cargo). The vehicle can also be engineered to incorporate targeting ligands to alter target tissue specificity. In some embodiments, the vehicle is a biological liposome. For example, the biological liposome is a phospholipid-based particle derived from human cells (e.g., erythrocyte ghosts, which are red blood cells broken down into spherical structures derived from the subject and wherein tissue targeting can be achieved by attachment of various tissue or cell-specific ligands), secretory exosomes, or subject derived membrane-bound nanovesicles (30−100 nm) of endocytic origin (e.g., can be produced from various cell types and can therefore be taken up by cells without the need for targeting ligands).

In some embodiments, the methods of modified TILs described herein comprise obtaining a population of immune effector cells from a sample. In some embodiments, a sample comprises a tissue sample, a fluid sample, a cell sample, a protein sample, or a DNA or RNA sample. In some embodiments, a tissue sample may be derived from any tissue type including, but not limited to skin, hair (including roots), bone marrow, bone, muscle, salivary gland, esophagus, stomach, small intestine (e.g., tissue from the duodenum, jejunum, or ileum), large intestine, liver, gallbladder, pancreas, lung, kidney, bladder, uterus, ovary, vagina, placenta, testes, thyroid, adrenal gland, cardiac tissue, thymus, spleen, lymph node, spinal cord, brain, eye, ear, tongue, cartilage, white adipose tissue, or brown adipose tissue. In some embodiments, a tissue sample may be derived from a cancerous, pre-cancerous, or non-cancerous tumor. In some embodiments, a fluid sample comprises buccal swabs, blood, plasma, oral mucous, vaginal mucous, peripheral blood, cord blood, saliva, semen, urine, ascites fluid, pleural fluid, spinal fluid, pulmonary lavage, tears, sweat, semen, seminal fluid, seminal plasma, prostatic fluid, pre-ejaculatory fluid (Cowper's fluid), excreta, cerebrospinal fluid, lymph, cell culture media comprising one or more populations of cells, buffered solutions comprising one or more populations of cells, and the like.

In some embodiments, the sample is processed to enrich or isolate a particular cell type, such as an immune effector cell, from the remainder of the sample. In certain embodiments, the sample is a peripheral blood sample which is then subject to leukopheresis to separate the red blood cells and platelets and to isolate lymphocytes. In some embodiments, the sample is a leukopak from which immune effector cells can be isolated or enriched. In some embodiments, the sample is a tumor sample that is further processed to isolate lymphocytes present in the tumor (i.e., to isolate tumor infiltrating lymphocytes).

In some embodiments, the isolated immune effector cells are expanded in culture to produce an expanded population of immune effector cells. One or more activating or growth factors may be added to the culture system during the expansion process. For example, in some embodiments, one or more cytokines (such as IL-2 and/or IL-7) can be added to the culture system to enhance or promote cell proliferation and expansion. In some embodiments, one or more activating antibodies, such as an anti-CD3 antibody, may be added to the culture system to enhance or promote cell proliferation and expansion. In some embodiments, the immune effector cells may be co-cultured with feeder cells during the expansion process. In some embodiments, the methods provided herein comprise one or more expansion phases. For example, in some embodiments, the immune effector cells can be expanded after isolation from a sample, allowed to rest, and then expanded again. In some embodiments, the immune effector cells can be expanded in one set of expansion conditions followed by a second round of expansion in a second, different, set of expansion conditions. Previous methods for ex vivo expansion of immune cells are known in the art, for example, as described in US Patent Application Publication Nos. 20180282694 and 20170152478 and U.S. Pat. Nos. 8,383,099 and 8,034,334.

At any point during the culture and expansion process, the gene-regulating systems described herein can be introduced to the immune effector cells to produce a population of modified TILs. In some embodiments, the gene-regulating system is introduced to the population of immune effector cells immediately after enrichment from a sample. In some embodiments, the gene-regulating system is introduced to the population of immune effector cells before, during, or after the one or more expansion process. In some embodiments, the gene-regulating system is introduced to the population of immune effector cells immediately after enrichment from a sample or harvest from a subject, and prior to any expansion rounds. In some embodiments, the gene-regulating system is introduced to the population of immune effector cells after a first round of expansion and prior to a second round of expansion. In some embodiments, the gene-regulating system is introduced to the population of immune effector cells after a first and a second round of expansion.

In some embodiments, the modified TILs produced by the methods described herein may be used immediately. Alternatively, the cells may be frozen at liquid nitrogen temperatures and stored for long periods of time, being thawed and capable of being reused. In such cases, the cells will usually be frozen in 10% dimethylsulfoxide (DMSO), 50% serum, 40% buffered medium, or some other such solution as is commonly used in the art to preserve cells at such freezing temperatures, and thawed in a manner as commonly known in the art for thawing frozen cultured cells.

In some embodiments, the modified TILs may be cultured in vitro under various culture conditions. The cells may be expanded in culture, i.e. grown under conditions that promote their proliferation. Culture medium may be liquid or semi-solid, e.g. containing agar, methylcellulose, etc. The cell population may be suspended in an appropriate nutrient medium, such as Iscove's modified DMEM or RPMI 1640, normally supplemented with fetal calf serum (about 5-10%), L-glutamine, a thiol, particularly 2-mercaptoethanol, and antibiotics, e.g. penicillin and streptomycin. The culture may contain growth factors to which the regulatory T cells are responsive. Growth factors, as defined herein, are molecules capable of promoting survival, growth and/or differentiation of cells, either in culture or in the intact tissue, through specific effects on a transmembrane receptor. Growth factors include polypeptides and non-polypeptide factors.

### A. Producing Modified TILs Using CRISPR/Cas Systems

In some embodiments, a method of producing a modified immune effector cell involves contacting a target DNA sequence with a complex comprising a gRNA and a Cas polypeptide. As discussed above, a gRNA and Cas polypeptide form a complex, wherein the DNA-binding domain of the gRNA targets the complex to a target DNA sequence and wherein the Cas protein (or heterologous protein fused to an enzymatically inactive Cas protein) modifies target DNA sequence. In some embodiments, this complex is formed intracellularly after introduction of the gRNA and Cas protein (or polynucleotides encoding the gRNA and Cas proteins) to a cell. In some embodiments, the nucleic acid encoding the Cas protein is a DNA nucleic acid and is introduced to the cell by transduction. In some embodiments, the Cas and gRNA components of a CRISPR/Cas gene editing system are encoded by a single polynucleotide molecule. In some embodiments, the polynucleotide encoding the Cas protein and gRNA component are comprised in a viral vector and introduced to the cell by viral transduction. In some embodiments, the Cas9 and gRNA components of a CRISPR/Cas gene editing system are encoded by different polynucleotide molecules. In some embodiments, the polynucleotide encoding the Cas protein is comprised in a first viral vector and the polynucleotide encoding the gRNA is comprised in a second viral vector. In some aspects of this embodiment, the first viral vector is introduced to a cell prior to the second viral vector. In some aspects of this embodiment, the second viral vector is introduced to a cell prior to the first viral vector. In such embodiments, integration of the vectors results in sustained expression of the Cas9 and gRNA components. However, sustained expression of Cas9 may lead to increased off-target mutations and cutting in some cell types. Therefore, in some embodiments, an mRNA nucleic acid sequence encoding the Cas protein may be introduced to the population of cells by transfection. In such embodiments, the expression of Cas9 will decrease over time, and may reduce the number of off target mutations or cutting sites.

In some embodiments, this complex is formed in a cell-free system by mixing the gRNA molecules and Cas proteins together and incubating for a period of time sufficient to allow complex formation. This pre-formed complex, comprising the gRNA and Cas protein and referred to herein as a CRISPR-ribonucleoprotein (CRISPR-RNP) can then be introduced to a cell in order to modify a target DNA sequence. The complexing can also occur in the target cell, with the Cas protein and gRNA being introduced separately.

B. Producing Modified TILs Using shRNA Systems

In some embodiments, a method of producing a modified immune effector cell by introducing into the cell one or more DNA polynucleotides encoding one or more shRNA molecules with sequence complementary to the mRNA transcript of a target gene is disclosed. The immune effector cell can be modified to produce the shRNA by introducing specific DNA sequences into the cell nucleus via a small gene cassette. Both retroviruses and lentiviruses can be used to introduce shRNA-encoding DNAs into immune effector cells. The introduced DNA can either become part of the cell's own DNA or persist in the nucleus, and instructs the cell machinery to produce shRNAs. shRNAs may be processed by Dicer or AGO2-mediated slicer activity inside the cell to induce RNAi mediated gene knockdown.

C. Producing Modified TILs Using siRNA Systems

In some embodiments, a method of producing a modified immune effector cell by introducing into the cell one or more DNA polynucleotides encoding one or more siRNA molecules with sequence complementary to the mRNA transcript of a target gene is disclosed. The immune effector cell can be modified to produce the siRNA by introducing specific DNA sequences into the cell nucleus via a small gene cassette. Retrovirus, adeno-associated virus, adenovirus, and lentivirus can be used to introduce siRNA-encoding DNAs into immune effector cells. The introduced DNA can either become part of the cell's own DNA or persist in the nucleus, and instructs the cell machinery to produce siRNAs. The siRNA can interfere with gene expression.

### IV. Adoptive Cell Transfer

Adoptive cell transfer (ACT) is a very effective form of immunotherapy and involves the transfer of immune cells with antitumor activity into cancer patients. ACT is a treatment approach that involves the identification, in vitro, of lymphocytes with antitumor activity, the in vitro expansion of these cells to large numbers and their infusion into the cancer-bearing host. Lymphocytes used for adoptive transfer can be derived from the stroma of resected tumors (tumor infiltrating lymphocytes or TILs). TILs for ACT can be prepared as described herein. TILs can be derived or from blood if they are genetically engineered to express antitumor T-cell receptors (TCRs) or chimeric antigen receptors (CARs), enriched with mixed lymphocyte tumor cell cultures (MLTCs), or cloned using autologous antigen presenting cells and tumor derived peptides. ACT in which the lymphocytes originate from the cancer-bearing host to be infused is termed autologous ACT. U.S. Publication No. 2011/0052530, incorporated by reference herein in its entirety, relates to a method for performing adoptive cell therapy to promote cancer regression, primarily for treatment of patients suffering from metastatic melanoma, which is incorporated by reference in its entirety for these methods. In some embodiments, TILs can be administered as described herein. In some embodiments, TILs can be administered in a single dose. Such administration may be by injection, e.g., intravenous injection. In some embodiments, TILs and/or cytotoxic lymphocytes may be administered in multiple doses.

Prior to transfer immune cells with antitumor activity into cancer patients, a lymphodepletion step on the patient may be utilized. The lymphodepletion eliminate partially or completely regulatory T cells and competing elements of the immune system. In some embodiments, lymphodepletion is utilized. In other embodiments, lymphodepletion is not utilized.

### V. Antibodies and Antibody Complexes

### A. Antibody Complexes

Unlike immobilized antibodies, soluble antibody complexes may provide a gentler activation signal to T cells. U.S. publication no. US 2007/0036783, incorporated herein by reference in its entirety, describes the use of soluble bispecific tetrameric antibody complexes (TAC) composed of one anti-CD3 antibody in complex with a second antibody against CD28 that can initiate T cell activation and expansion.

The present disclosure provides methods of activating TILs comprising culturing a sample containing TILs with a composition comprising at least one soluble monospecific complex, wherein each soluble monospecific complex comprises two binding proteins which are linked and specifically bind to the same antigen on the TILs. In certain embodiments, the TILs are cultured in the presence of IL-2. In certain embodiments, the two binding proteins are the same binding protein and bind to the same epitope on the antigen.

In certain embodiments, the binding proteins are antibodies or fragments thereof. Antibody fragments that may be used include Fab, Fab′, F(ab)₂, scFv and dsFv fragments from recombinant sources and/or produced in transgenic animals. The antibody or fragment may be from any species including mice, rats, rabbits, hamsters and humans. Chimeric antibody derivatives, i.e., antibody molecules that combine a non-human animal variable region and a human constant region are also contemplated within the scope of the invention. Chimeric antibody molecules can include, for example, humanized antibodies which comprise the antigen binding domain from an antibody of a mouse, rat, or other species, with human constant regions. Conventional methods may be used to make chimeric antibodies. (See, for example, Morrison et al.; Takeda et al., Cabilly et al., U.S. Pat. No. 4,816,567; Boss et al., U.S. Pat. No. 4,816,397; Tanaguchi et al., European Patent Publication EP171496; European Patent Publication 0173494, United Kingdom patent GB 2177096B). The preparation of humanized antibodies is described in EP-B 10 239400. Humanized antibodies can also be commercially produced (Scotgen Limited, 2 Holly Road, Twickenham, Middlesex, Great Britain). It is expected that chimeric antibodies would be less immunogenic in a human subject than the corresponding non-chimeric antibody. The humanized antibodies can be further stabilized for example as described in WO 00/61635. All of these publications are incorporated by reference herein in their entireties.

Antibodies or fragments thereof that bind to TIL antigens are available commercially or may be prepared by one of skill in the art. In one embodiment, the two antibodies or fragments thereof which bind to the same antigen are linked directly. Direct linking of the antibodies may be prepared by chemically coupling one antibody to the other, for example by using N-succinimidyl-3-(2-pyridyldithio) propionate (SPDP).

In another embodiment, the two antibodies are indirectly linked in the soluble monospecific complex. By “indirectly linked” it is meant that the two antibodies are not directly covalently linked to each other but are attached through a linking moiety such as an immunological complex. In a preferred embodiment, the two antibodies are indirectly linked by preparing a tetrameric antibody complex. A tetrameric antibody complex may be prepared by mixing monoclonal antibodies that bind to the same antigen and are of the same animal species with approximately an equimolar amount of monoclonal antibodies of a second animal species which are directed against the Fc-fragments of the antibodies of the first animal species. The first and second antibody may also be reacted with an about equimolar amount of the F(ab′)2 fragments of monoclonal antibodies of a second animal species which are directed against the Fc-fragments of the antibodies of the first animal species. See U.S. Pat. No. 4,868,109 to Lansdorp, which is incorporated herein by reference in its entirety, for a description of tetrameric antibody complexes and methods for preparing same.

In one embodiment, the composition comprises at least two different monospecific complexes, each binding to a different antigen on the TILs. In one embodiment, the composition comprises at least two different soluble monospecific complexes and each of the at least two different soluble monospecific complexes binds to a different antigen selected from the group consisting of CD3, CD28, CD2, CD7, CD11a, CD26, CD27, CD30L, CD40L, OX-40, ICES, GITR, CD137, and HLA-DR.

In a specific embodiment, one monospecific complex will bind CD3 and the second monospecific complex will bind CD28. In another embodiment, the composition comprises at least three different soluble monospecific complexes, each binding to one of three different antigens on the TILs. In such embodiment, no two monospecific complexes will bind the same antigen. In a specific embodiment, the composition comprises three different soluble monospecific complexes, one specific for CD3, a second specific for CD28 and a third specific for CD2. In a specific embodiment, the activation of TILs in the presence of the soluble monospecific complexes is greater than the activation of TILs using a bispecific complex comprising two different binding proteins or antibodies, each of which binds to a different antigen on the TILs.

### B. Anti-CD3 and Anti-CD28 Antibodies

As will be appreciated by those in the art, there are a number of suitable anti-human CD3 and anti-human CD28 antibodies that find use in the invention. Anti-human CD3 antibodies include polyclonal and monoclonal antibodies from various mammals, including, but not limited to, murine, human, primate, rat, and canine antibodies. In particular embodiments, the OKT3 anti-CD3 antibody is used (commercially available from Ortho-McNeil, Raritan, N.J. or Miltenyi Biotech, Auburn, Calif.). As it will be also appreciated by those in the art, there are a number of suitable anti-human CD28 antibodies that find use in the invention, including anti-human CD28 polyclonal and monoclonal antibodies.

Antibodies against CD3 are a central element in many T cell proliferation protocols. Immobilized on a surface, anti-CD3 delivers an activating and proliferation-inducing signal by crosslinking of the T cell receptor complex on the surface of T cells. By immobilizing anti-CD3 and anti-CD28 to simultaneously deliver a signal and a co-stimulatory signal, proliferation can be increased (Baroja et al (1989), Cellular Immunology, 120: 205-217). In WO09429436A1 solid phase surfaces such as culture dishes and beads are used to immobilize the anti-CD3 and anti-CD28 antibodies. Regularly, the immobilization on beads is performed on DynaBeads®M-450 having a size of 4.5 m in diameter. All of these publications are incorporated by reference herein in their entireties.

### VI. Cytokines

The expansion methods described herein generally use culture media with high doses of a cytokine, in particular IL-2, as is known in the art.

As used herein, the term “IL-2” (also referred to herein as “IL2”) refers to the cytokine and T cell growth factor known as interleukin-2, and includes all forms of IL-2, including human and mammalian forms, forms with conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated herein by reference in their entireties. The term IL-2 encompasses human, recombinant forms of IL-2, such as aldesleukin (PROLEUKIN, available commercially from multiple suppliers in 22 million IU per single use vials), as well as the form of recombinant IL-2 commercially supplied by CellGenix, Inc., Portsmouth, N.H., USA (CELLGRO GMP) or ProSpec-Tany TechnoGene Ltd., East Brunswick, N.J., USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The term IL-2 also encompasses pegylated forms of IL-2, including the pegylated IL-2 prodrug NKTR-214, available from Nektar Therapeutics, South San Francisco, Calif., USA. NKTR-214 and pegylated IL-2 suitable for use in the invention is described in U.S. Patent Application Publication No. US 2014/0328791 A1 and International Patent Application Publication No. WO 2012/065086 A1, the disclosures of which are incorporated herein by reference in their entireties. Alternative forms of conjugated IL-2 suitable for use in the invention are described in U.S. Pat. Nos. 4,766,106, 5,206,344, 5,089,261 and 4,902,502, the disclosures of which are incorporated herein by reference in their entireties. Formulations of IL-2 suitable for use in the invention are described in U.S. Pat. No. 6,706,289, the disclosure of which is incorporated herein by reference in its entirety. The human IL2 gene is identified by NCBI Gene ID 3558. An exemplary nucleotide sequence for a human IL2 gene is the NCBI Reference Sequence: NG_016779.1. An exemplary amino acid sequence of a human IL-2 polypeptide is provided as SEQ ID NO: 879.

Interleukin-2 (IL-2) is an interleukin, a type of cytokine signaling molecule in the immune system. It is a 15.5-16 kDa protein that regulates the activities of white blood cells (leukocytes, often lymphocytes) that are responsible for immunity. IL-2 is part of the body's natural response to microbial infection. IL-2 mediates its effects by binding to IL-2 receptors, which are expressed by lymphocytes. The major sources of IL-2 are activated CD4+ T cells and activated CD8+ T cells.

IL-2 has essential roles in key functions of the immune system, tolerance and immunity, primarily via its direct effects on T cells. In the thymus, where T cells mature, it prevents autoimmune diseases by promoting the differentiation of certain immature T cells into regulatory T cells, which suppress other T cells that are otherwise primed to attack normal healthy cells in the body. IL-2 enhances activation-induced cell death (AICD). IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes while it impedes differentiation into Th17 and follicular Th lymphocytes. Its expression and secretion is tightly regulated and functions as part of both transient positive and negative feedback loops in mounting and dampening immune responses. Through its role in the development of T cell immunologic memory, which depends upon the expansion of the number and function of antigen-selected T cell clones, it plays a role in enduring cell-mediated immunity.

### VII. Pharmaceutical Compositions, Dosages, and Dosing Regimens

In an embodiment, TILs expanded using the methods of the present disclosure are administered to a patient as a pharmaceutical composition. In an embodiment, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. In some embodiments, from about 1×10⁹ to about 2×10¹¹ of TILs are administered. In some embodiments, from about 2.3×10¹⁰ to about 13.7×10¹⁰ TILs are administered, with an average of around 7.8×10¹⁰ TILs, particularly if the cancer is melanoma. In an embodiment, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, the therapeutically effective dosage is about 2.3×10¹⁰ to about 13.7×10¹⁰. In some embodiments, the therapeutically effective dosage is about 7.8×10¹⁰ TILs, particularly of the cancer is melanoma. In some embodiments, the therapeutically effective dosage is about 1.2×10¹⁰ to about 4.3×10¹⁰ of TILs. In some embodiments, the therapeutically effective dosage is about 3×10¹⁰ to about 12×10¹⁰ TILs. In some embodiments, the therapeutically effective dosage is about 4×10¹⁰ to about 1×10¹⁰ TILs. In some embodiments, the therapeutically effective dosage is about 5×10¹⁰ to about 8×10¹⁰ TILs. In some embodiments, the therapeutically effective dosage is about 6×10¹⁰ to about 8×10¹⁰ TILs. In some embodiments, the therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In an embodiment, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×107, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹¹, 4×10¹², 5×10¹², 6×10¹², 7×10¹¹, 8×10¹², 9×10¹¹, 1×10¹³, 2×10¹³, 3×10¹³, 4×10¹³, 5×10¹³, 6×10¹³, 7×10¹³, 8×10¹³, and 9×10¹³ cells. In some embodiments, an effective dosage of TILs is in the range of 1×10⁶to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10″ to 1×10¹², 1×10¹²to 5×10¹², and 5×10″ to 1×10¹³ cells.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation. In certain embodiments, TILs are administered intravenously.

### VIII. Cell Counts, Cell Viability, Flow Cytometry

In some embodiments, cell counts and/or viability are measured. The expression of markers such as but not limited CD3, CD4, CD8, and CD56, as well as any other disclosed or described herein, can be measured by flow cytometry with antibodies, for example but not limited to those commercially available from BD Bio-sciences (BD Biosciences, San Jose, Calif.) using a FACSCanto™ flow cytometer (BD Biosciences). The cells can be counted manually using a disposable c-chip hemocytometer (VWR, Batavia, Ill.) and viability can be assessed using any method known in the art, including but not limited to trypan blue staining.

In an embodiment, a method for expanding TILs may include using about 5,000 mL to about 25,000 mL of cell medium, about 5,000 mL to about 10,000 mL of cell medium, or about 5,800 mL to about 8,700 mL of cell medium. In an embodiment, expanding the number of TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad Calif.).

In an embodiment, TILs are expanded in gas-permeable containers. Gas-permeable containers have been used to expand TILs using PBMCs using methods, compositions, and devices known in the art, including those described in U.S. Patent Application Publication No. 2005/0106717 A1, the disclosure of which is incorporated herein by reference in its entirety. In an embodiment, TILs are expanded in gas-permeable bags. In an embodiment, TILs are expanded using a cell expansion system that expands TILs in gas permeable bags, such as the Xuri Cell Expansion System W25 (GE Healthcare). In an embodiment, TILs are expanded using a cell expansion system that expands TILs in gas permeable bags, such as the WAVE Bioreactor System, also known as the Xuri Cell Expansion System W5 (GE Healthcare). In an embodiment, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L. In an embodiment, TILs can be expanded in G-Rex flasks (commercially available from Wilson Wolf Manufacturing). Such embodiments allow for cell populations to expand from about 5×10⁵ cells/cm² to between 10×10⁶ and 30×10⁶ cells/cm². In an embodiment this expansion is conducted without adding fresh cell culture media to the cells (also referred to as feeding the cells). In an embodiment, this is without feeding so long as medium resides at a height of about 10 cm in the G-Rex flask. In an embodiment this is without feeding but with the addition of one or more cytokines. In an embodiment, the cytokine can be added as a bolus without any need to mix the cytokine with the medium. Such containers, devices, and methods are known in the art and have been used to expand TILs, and include those described in U.S. Patent Application Publication No. US 2014/0377739A1, International Publication No. WO 2014/210036 A1, U.S. Patent Application Publication No. us 2013/0115617 A1, International Publication No. WO 2013/188427 A1, U.S. Patent Application Publication No. US 2011/0136228 A1, U.S. Pat. No. 8,809,050 B2, International publication No. WO 2011/072088 A2, U.S. Patent Application Publication No. US 2016/0208216 A1, U.S. Patent Application Publication No. US 2012/0244133 A1, International Publication No. WO 2012/129201 A1, U.S. Patent Application Publication No. US 2013/0102075 A1, U.S. Pat. No. 8,956,860 B2, International Publication No. WO 2013/173835 A1, U.S. Patent Application Publication No. US 2015/0175966 A1. Such processes are also described in Jin et al., J. Immunotherapy, 2012, 35:283-292. All of these publications are incorporated by reference herein in their entireties.

### EXAMPLES

### Example 1: Methods for Expanding Tumor Infiltrating Lymphocytes (TILs)

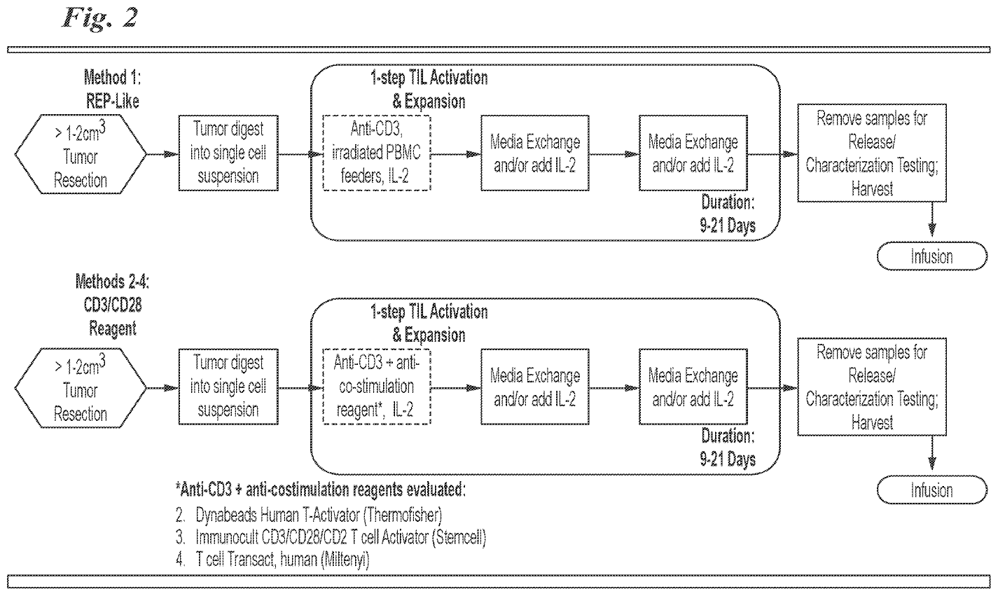

Tumor infiltrating lymphocytes (TILs) were expanded directly from single cell suspensions of primary human melanoma metastases. TILs were obtained from three different donors: Donor 1 (D3239), Donor 2 (D3399), and Donor 3 (D6755). On Day 0 of the culture, 200,000-800,000 live cells from single cell suspensions were taken and seeded into the wells of a 24 well Grex plate (Wilson Wolf, Cat #80192M) in a 6 mL volume of TIL media (a 1:1 mixture of RPMI 1640 and AIM V, supplemented with 5% human AB serum) and supplemented with 6,000 U/ml of recombinant human IL-2 (CellGenix, Cat #1020-1000). The viable cells seeded per condition contained 3.4K to 52K CD3+ T cells as determined by flow cytometry. Cells were activated and expanded using four different methods, as described below (FIG. 2):
TIL Expansion Method 1 (“REP-like”)—One-step Rapid Expansion Protocol without pre-REP. Feeder cells (PBMCs) from five healthy donors were irradiated (6,000 rads) and pooled in a 1:1:1:1:1 ratio. 10M irradiated PBMCs were added to each well, as well as 360 ng of OKT3 (Biolegend, Cat #317326) for a final concentration of 60 ng/ml.
TIL Expansion Method 2 (“Dynabeads”)—Dynabeads were added at 1.5 or 0.5×10⁶ beads/well. Dynabeads having anti-CD3 and anti-CD28 antibodies conjugated to their surfaces were added to the TILs.
TIL Expansion Method 3 (“Stemcell”)—Tetrameric Antibody Complexes (TAC) from Stemcell. 37.5 μl of anti-CD3/anti-CD2/anti-CD28 tetrameric antibody complex (TAC) from Stemcell Technologies (Cat #10970) was added to the TILs, for a final concentration of 6.25 μl/ml.
TIL Expansion Method 4 (“Transact”)—Nanomatrix from Miltenyi Biotec. 324 μl of a colloidal polymeric nanomatrix covalently attached to humanized recombinant agonists against human CD3 and CD28 from Miltenyi Biotec (MACS GMP T Cell Transact, Cat #130-019-011) was added to the TILs, for a final concentration of 54 μl/ml.

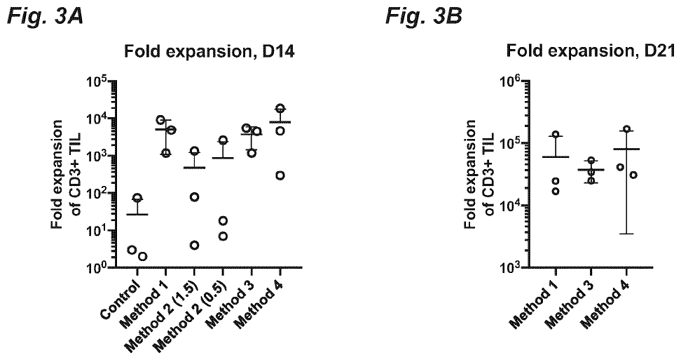

For all four TIL expansion methods outlined above, a common protocol was followed at discrete time intervals with variations for each method indicated below (FIG. 2):
Day 2: 36,000 U of recombinant human IL-2 was added to each well, for a final concentration of 6,000 U/ml, assuming consumption.
Day 4: 50% media was replaced/exchanged. For method 1, from each well, 4 mL of cell supernatant was removed and discarded, being careful not to disturb the cells at the bottom of the well. Subsequently, 4 mL of fresh TIL media and 35,000 U of IL-2 was then added, for a final concentration of 6,000 U/ml, assuming consumption. For method 2, cells were harvested and placed in a magnet to remove Dynabeads, then centrifuged at 300×g for 5 minutes. For methods 3 and 4, cultures were harvested, and TILs centrifuged at 300×g for 5 minutes. The cell supernatant was aspirated and each of the cell cultures were resuspended in 6 mL of TIL media +6,000 U/ml IL-2, then seeded back into the well of a 24 well G-rex plate.
Day 7: IL-2 was added to the media for a final concentration of 6,000 U/ml, assuming consumption. When cultures reached greater than 5M cells (reflecting greater than 2.5M cells per cm² of the Grex membrane), they were transferred to the wells of a 6-well Grex plate (Wilson wolf), and brought up to 100 mL with TIL media supplemented with 6000 U/ml IL-2.
Day 9: 50% media was replaced/exchanged as in Day 4.
Day 12: IL-2 was added to the media for a final concentration of 6,000 U/ml, assuming consumption. When cultures reached greater than 5M cells (reflecting greater than 2.5M cells per cm² of the Grex membrane), they were transferred to the wells of a 6-well Grex plate (Wilson wolf), and brought up to 100 mL with TIL media supplemented with 6,000 U/ml IL-2.
Day 14: 10M cells were taken from the cultures and reseeded into a Grex-10 vessel, along with 40 ml TIL media+6,000 U/ml IL-2. As shown in FIG. 3A, at 14 days, on average, cell cultures grown in IL-2 alone (labeled ‘Control’) expanded ˜27-fold, cells grown by Method 1 (REP-like) expanded 5063-fold, cells grown by Method 3 (‘Stemcell’) expanded 3755-fold, and cells grown by Method 4 (‘Transact’) expanded 7924-fold.
Day 16: 50% media was replaced/exchanged as in Day 4.
Day 17: IL-2 was added to the media for a final concentration of 6,000 U/ml, assuming consumption.
Day 21: cell cultures were harvested. As shown in FIG. 3B, by day 21, on average, cells grown by Method 1 (REP-like) expanded 60166-fold, cells grown by Method 3 (‘Stemcell’) expanded 37596-fold, and cells grown by Method 4 (‘Transact’) expanded 80373-fold.

### Example 2: Phenotypic Characterization of TILs

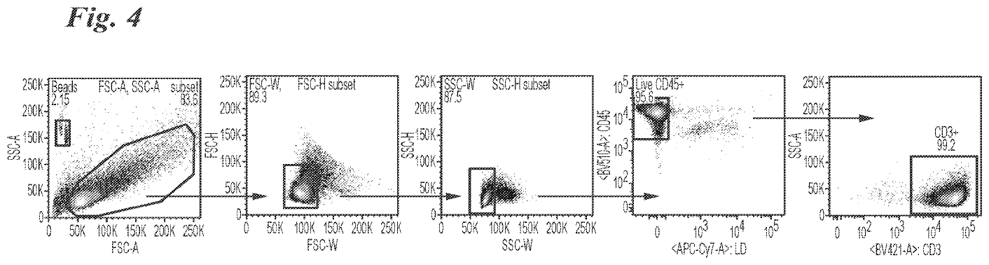

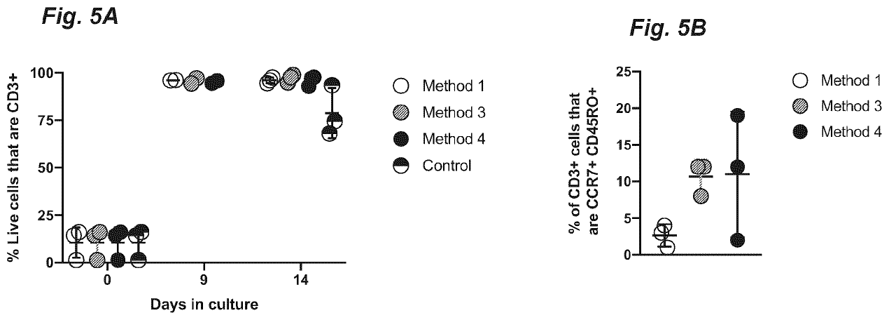

The cellular composition of TILs as a function of time during the TIL expansion process was assessed by flow cytometry (FIG. 4). Cells were cultured as in Example 1, and an aliquot of cells was stained with antibodies to detect cells that express CD45 and CD3e on days 0, 9, and 14. A progressive enrichment of CD45+ CD3+ T cells over time was observed for all donors (FIG. 5A), demonstrating specific expansion of T cells when using Methods 1, 3, and 4 described in Example 1.

Furthermore, the phenotype of T cells produced was assessed. In particular, the proportion of cells that were defined as central memory T cell phenotype (Tem, with marker phenotype CD45RO+ CCR7+ CD45RA−) was determined by flow cytometry. Cells cultured as in Example 1 were taken, and on day 14 an aliquot of cells was stained with fluorescently labeled antibodies that detect CD45RO, CCR7, and CD45RA. Compared to Method 1 (REP-like) using feeder cells, Method 3 (Stemcell) and Method 4 (Transact) generated greater proportions of Tcm (FIG. 5B).

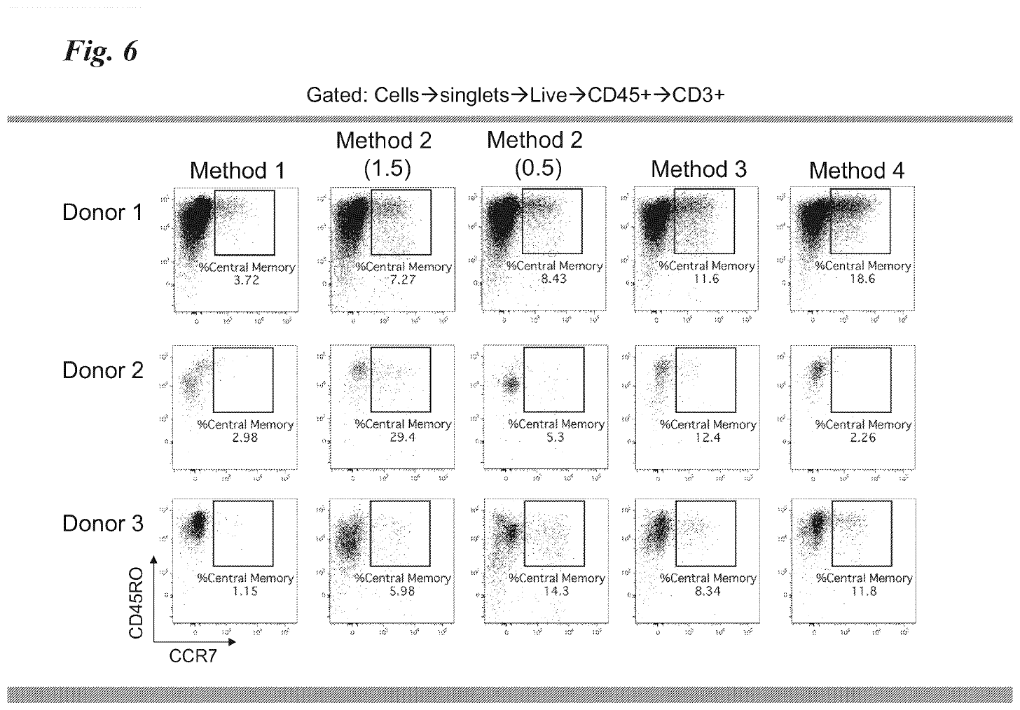

TILs were analyzed by FACS for CD45RO and CCR7 co-expression, markers for the central memory T cell phenotype (FIG. 6). For Donor 3, TILs expanded using any of the three feeder cell-free TIL expansion methods described in Example 1 (Method 2 (‘Dynabeads’ using 1.5 or 0.5×10⁶ beads/well), Method 3 (‘Stemcell’), and Method 4 (‘Transact’)), showed greater enrichment in cells having the central memory T cell phenotype, between about 6% and 14%, as compared to TILs expanded using Method 1 (‘REP’), where the central memory T cell phenotype constituted only about 1%. For Donor 2, TILs expanded using any of the three feeder cell-free TIL expansion methods described in Example 1 (Method 2 (‘Dynabeads’ using 1.5 or 0.5×10⁶ beads/well), Method 3 (‘Stemcell’), and Method 4 (‘Transact’)), showed greater enrichment in cells having the central memory T cell phenotype, between about 5% and 29%, as compared to TILs expanded using Method 1 (‘REP-like’), where the central memory T cell phenotype constituted only about 3%. For Donor 1, TILs expanded using any of the three feeder cell-free TIL expansion methods described in Example 1 (Method 2 (‘Dynabeads’ using 1.5 or 0.5×10⁶ beads/well), Method 3 (‘Stemcell’), and Method 4 (‘Transact’)), showed greater enrichment in cells having the central memory T cell phenotype, between about 7% and 19%, as compared to TILs expanded using Method 1 (‘REP-like’ feeder cell-based), where the central memory T cell phenotype constituted only about 4%. The % central memory T cell phenotype data for TILs expanded by all four methods using TILs from all three donors were summarized, showing that Method 4 (‘Transact’) produced TILs significantly enriched in central memory T cell phenotype for two out of three donors (FIG. 6).

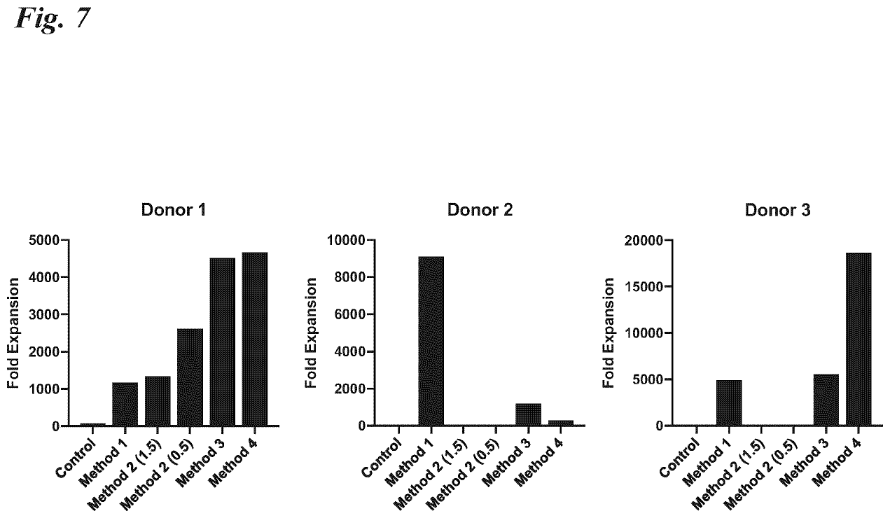

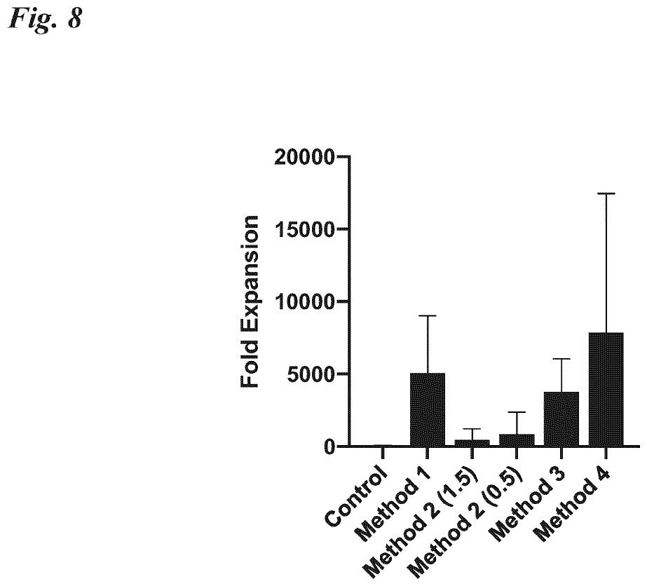

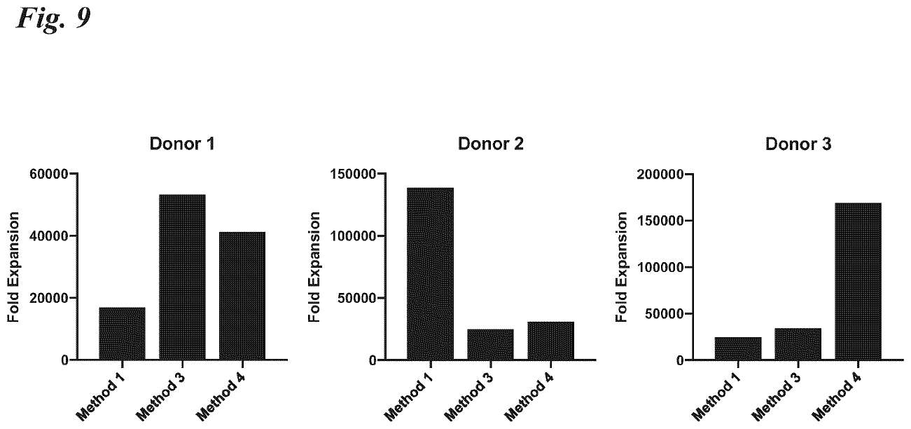

The fold expansion (FIG. 7) of TILs at day 14 (relative to the number of cells on Day 0) was assessed for TILs expanded by the addition of IL-2 alone (‘Control’) and all four methods described in Example 1 (Method 1 (‘REP-like’), Method 2 (‘Dynabeads’ using 1.5 or 0.5×10⁶ beads/well), Method 3 (‘Stemcell’), and Method 4 (‘Transact’)). For Donors 3 and 1, Method 4 resulted in about 4-5 times greater expansion as compared to Method 1 (feeder cell-based), while for Donor 2, Method 1 resulted in about 20 times greater expansion as compared to Method 4. Method 3 resulted in about 4.5 times greater expansion as compared to Method 1 (feeder cell-based) for Donor 1 (FIG. 7). The data were combined for all three donors, showing that, on average, TILs expanded by Method 3 and Method 4 showed a fold expansion at least as high, if not higher, than TILs expanded by Method 1 (REP-like) (FIG. 8). Some cells were cultured past Day 14 to assess if further expansion was possible. Cells continued to expand between days 14 and 21, in some instances demonstrating a total fold expansion of greater than 150,000 (FIG. 9).

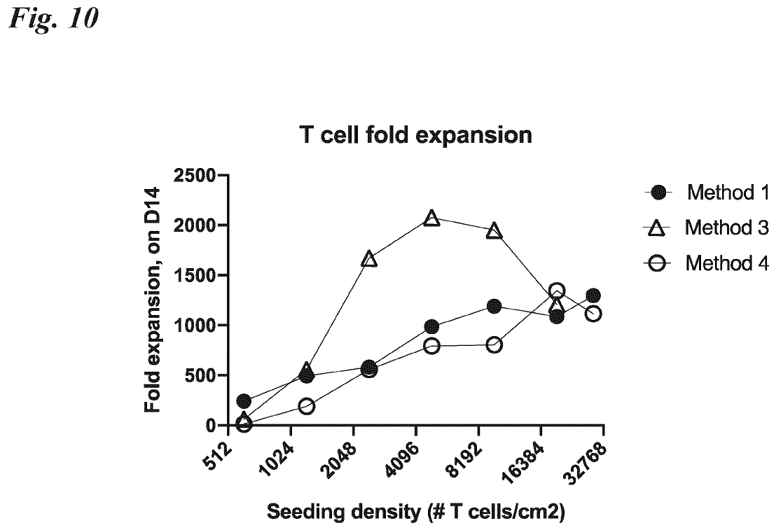

Tumors are heterogenous and contain varying percentages of T cells; a robust T cell expansion method must be able to expand cells at both high and low T cell densities. It was unclear whether low seeding densities of T cells could be expanded in the absence of feeder cells. Thus, impact of the seeding density of the T cells on day 0 on the fold expansion of TILs by Day 14 was assessed. The fold expansion increased relatively linearly for TILs expanded by Method 1 (‘REP-like’) and Method 3 (‘Stemcell’), but peaked at seeding densities of between about 2,000 T cells/cm² and about 9,000 T cells/cm² for TILs expanded by Method 4 (‘Transact’) (FIG. 10). Thus, Method 4 was about equivalent to method 1 at expanding T cells at low density, and Method 3 was better than method 1 at lower densities. This demonstrates that feeder cells are not intrinsically required to expand T cells at a density as low as 85 T cells per mL culture media (in a 6 mL volume).

Donor 3 had previously provided a sample for TIL expansion using a pre-REP/REP method. The sample from Donor 3 failed to provide an adequate number of TILs from the pre-REP step to proceed to the REP step. Samples from Donor 3 provided a much higher fold expansion of TILs than was achieved using pre-REP. This shows that the methods described herein can rescue a situation where in adequate expansion occurs during pre-REP.

### Example 3: Functionality of Feeder-Free Process Cells (Cytokine Production by TILs)

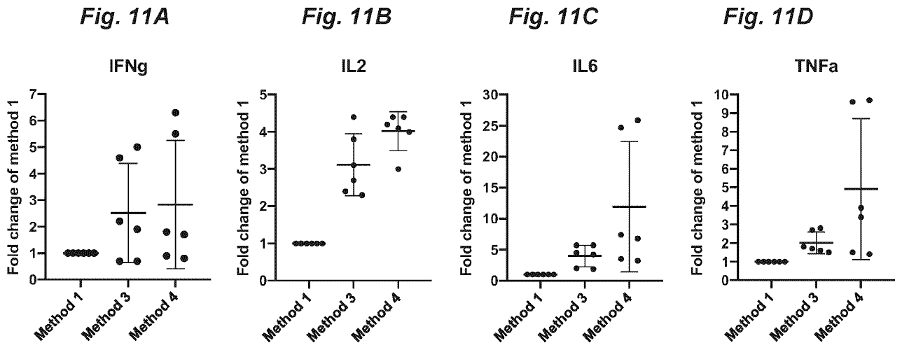

The production of the cytokines IL-2, IFNγ, and TNFα by TILs generated using feeder cell-free methods (TIL expansion Methods 3 and 4) was assessed. TILs were expanded from dissociated tissue cells as described in Example 1. On day 14 of the process, 200,000 expanded TILs (from three independent donors) were taken and stimulated for 18 hours in a 200 μl volume of TIL media that was additionally supplemented with 6.6 μl of a tetrameric anti-CD3 reagent (Stemcell Technologies, custom reagent). Subsequently, cell supernatants were harvested, and the concentration of cytokines in the supernatant was determined using duplicate samples with the Quickplex S120 (Mesoscale Discovery). As shown in FIG. 11, for the three different donors, TILs generated directly from dissociated tumor cells were able to produce IFNγ, IL-2, TNFα, and IL-6 upon re-stimulation. Additionally, TILs generated using feeder cell-free methods (Method 3: ‘Stemcell’) and Method 4: ‘Transact’) were able to generate up to ˜6-fold more IFNγ, ˜4-fold more IL-2, ˜10-fold more TNFα a, and ˜25-fold more IL-6 than TILs generated using Method 1 (‘REP-like’ using feeder cells) (FIG. 11).

### Example 4: Methods to Genetically Engineer TILs Using CRISPR-Cas9

TILs expanded using the protocols described in Example 1 were genetically engineered using CRISPR-Cas9 to create functional genetic knockouts of a target gene. This genetic engineering was performed on day 13 of each method described in Example 1, and also on other days, ranging from day 0-21. Briefly, on day 13, 1.2×10⁶ expanded TILs were centrifuged at 300×g for 5 minutes and resuspended with 40 μl of MaxCyte electroporation buffer (HyClone Cat #EPB1). A ribonucleoprotein (RNP) master mix was made containing 52 pmol Cas9 protein (Aldevron, Cat #9212) and 240 pmol of sgRNA targeting the PTPRC gene (IDT, GAGTTTAAGCCACAAATACA SEQ ID NO: 909), which encodes CD45 antigen. 100 μM solution of PTPRC sgRNA was made by resuspending 10 nmol lyophilized sgRNA with 100 μl Nuclease Free Duplex Buffer (IDT Cat #1072570). Reagents were added as follows:

| | Vol (μL) |
|---|---|
| sgRNA (100 μM) | 4.8 |
| Cas9 (61 μM) | 1.68 |
| DPBS (1X) | 3.52 |

The entire 10 μl of the RNP master mix was added to the 40 μl cell suspension. 50 μl of cell suspension was then transferred to an OC100×2 processing assembly (MaxCyte, Cat #SOC-1X2). Cells were electroporated on a MaxCyte ExPERT electroporator using the “Optimization #9” program. Subsequently, 50 μl TIL media was added to the well and cells were transferred to a 96-well plate containing 100 μl TIL media, which was then incubated at 37° C. for 20 minutes. Subsequently, cells were counted, and 500 K live cells were then seeded into a 24 well Grex plate containing 6 mL TIL media supplemented with 6,000 U/ml IL-2.

One day later (day 14 of the culture), IL-2 was added, assuming consumption, to 6,000 U/ml. On day 17 of the culture, 3 mL of cell supernatant from each well was removed and discarded (being careful not to disturb the cells at the bottom of the well). This was replaced with 3 mL of fresh TIL media and IL-2 was added to a final concentration of 6,000 U/ml assuming consumption. On day 18, IL-2 was added, assuming consumption, to 6,000 U/ml. On day 21, cells were harvested and counted. Cell pellets were frozen, and editing was determined by amplicon sequencing.

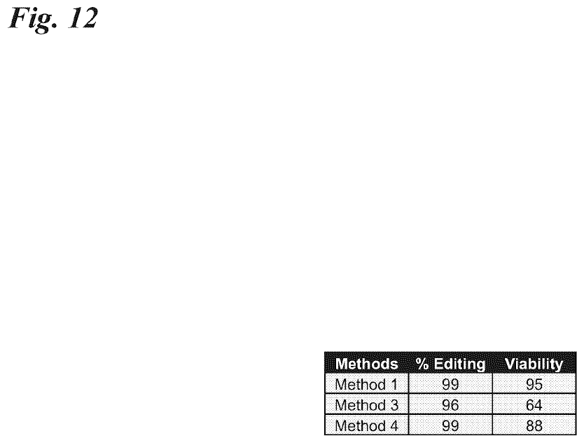

Cell viability and percent editing of CD45 was assessed (FIG. 12) for edited TILs versus TILs that were not genetically modified. Assays were performed 8 days after electroporation for TILs expanded using Method 1 (‘REP-like’ using feeder cells), Method 3 (‘Stemcell’), and Method 4 (‘Transact’).

### Example 5: Methods for Expanding Pre-REP Failure TILs with Soluble Activators or Artificial Antigen Presenting Cells (aAPCs)

Tumor infiltrating lymphocytes (TILs) were expanded directly from single cell suspensions of primary human melanoma metastases. TILs were obtained from three different donors: Donor 3239, Donor 6752, and Donor 6755. Donor 6752 and Donor 6755 were previously identified as pre-REP failures, unable to expand to 4×10⁷ cells in 23 days in a pre-REP. On Day 0 of the culture, 400,000-800,000 live cells from single cell suspensions were taken and seeded into the wells of a 24 well Grex plate (Wilson Wolf, Cat #80192M) in a 6 ml volume of TIL media (a 1:1 mixture of RPMI 1640 and AIM V, supplemented with 5% human AB serum) and supplemented with 6,000 U/ml of recombinant human IL-2 (Peprotech, Cat #200-02). The viable cells seeded per condition contained 22K to 52K CD3+ T cells as determined by flow cytometry. Cells were activated and expanded using five different methods, as described below:
TIL Expansion Method 1 (“REP-like”)—One-step Rapid Expansion Protocol without pre-REP. Feeder cells (PBMCs) from five healthy donors were irradiated (6,000 rads) and pooled in a 1:1:1:1:1 ratio. 1×10⁷ irradiated PBMCs were added to each well, as well as 360 ng of OKT3 (Biolegend, Cat #317326) for a final concentration of 60 ng/ml.
TIL Expansion Method 3 (“Stemcell”)—Tetrameric Antibody Complexes (TAC) from Stemcell. 37.5 μl of anti-CD3/anti-CD2/anti-CD28 tetrameric antibody complex (TAC) from Stemcell Technologies (Cat #10970) was added to the TILs, for a final concentration of 6.25 l/ml.
TIL Expansion Method 4 (“Transact”)—Nanomatrix from Miltenyi Biotec. 85 μl of a colloidal polymeric nanomatrix covalently attached to humanized recombinant agonists against human CD3 and CD28 from Miltenyi Biotec (MACS GMP T Cell Transact, Cat #170-076-156) was added to the TILs, for a final dilution of 70:1.
TIL Expansion Method 5 (“aAPC-OKT3”)—K562 cells engineered to express OKT3 were irradiated (15,000 rads). 1×10⁶ irradiated aAPC-OKT3 cells were added to each well for a final cell to area ratio of 5×10⁵ cells/cm².
TIL Expansion Method 6 (“aAPC-OKT3-CD86”)—K562 cells engineered to express OKT3 and CD86 were irradiated (15,000 rads). 1×10⁶ irradiated aAPC-OKT3 cells were added to each well for a final cell to area ratio of 5×10⁵ cells/cm².

For all five TIL expansion methods outlined above, a common protocol was followed at discrete time intervals with variations for each method indicated below:
Day 2: 36,000 U of recombinant human IL-2 was added to each well, for a final concentration of 6,000 U/ml, assuming consumption to the corresponding wells.
Day 4 and Day 6: 50% media was replaced/exchanged. From each well, 3 ml of cell supernatant was removed and discarded, being careful not to disturb the cells at the bottom of the well. Subsequently, 3 ml of fresh TIL media and 36,000 U of IL-2 was then added, for a final concentration of 6,000 U/ml, assuming consumption. Day 7: For all conditions, cells were counted. The entire volume (6 ml) was transferred to a 6 well Grex (Wilson Wolf) containing 100 ml TIL media with 6000 U/ml IL-2.
Day 10 or Day 11: On Day 10 and Day 11, all aAPC samples and soluble activator samples were engineered using CRISPR-Cas9 as described in Example 4 respectively. Following electroporation, 2×10⁵ cells were transferred to a 24 well Grex (Wilson Wolf) in 6 ml TIL media containing 6000 U/ml of IL-2.
Day 13: 36,000 U of recombinant human IL-2 was added to each well, for a final concentration of 6,000 U/ml, assuming consumption to the corresponding wells.
Day 15: 50% media was replaced/exchanged. From each well, 50 ml of cell supernatant was removed and discarded, being careful not to disturb the cells at the bottom of the well. Subsequently, 50 ml of fresh TIL media and 36,000 U of IL-2 was then added, for a final concentration of 6,000 U/ml, assuming consumption.
Day 18: Donor 3339 samples were harvested. Donor 6755 samples were harvested, samples olfactory (O), SOCS1 (S), and SOCS1+PTPN2 (S+P2) activated with aAPC-OKT3 or aAPC-OKT3-CD86 in IL-2 underwent a 50% media exchange as described on day 15. Donor 6752 samples underwent a 50% media exchange as described on day 15.
Day 21: Remaining Donor 6755 samples and Donor 6752 samples underwent a 50% media exchange as described on day 15.
Day 23: Remaining Donor 6755 samples and Donor 6752 samples were harvested.

### Example 6: Methods to Genetically Engineer Pre-REP Failure TILs with Soluble Activators or aAPCs Using CRISPR-Cas9

TILs expanded using the protocols described in Example 5 were genetically engineered using CRISPR-Cas9 to create functional genetic knockouts of a target gene. This genetic engineering was performed on day 10 of each method described in Example 5, and also on other days, ranging from day 0-21. Briefly, on day 10, 1.2×10⁶ expanded TILs were centrifuged at 300×g for 5 minutes and resuspended with 20 μl of MaxCyte electroporation buffer (HyClone Cat #EPB1). Several ribonucleoprotein (RNP) master mixes were made containing 52 pmol Cas9 protein (Aldevron, Cat #9212) and 120 pmol of each individual sgRNA. Master mix 1 contained the sgRNA for the OR1A2 gene (O) (IDT, AGATGATGTCAACCAAGGAG SEQ ID NO: 910). Master mix 2 contained the sgRNA for the SOCS1 gene (S) (IDT, GACGCCTGCGGATTCTACTG SEQ ID NO: 61). Master mix 3 contained sgRNAs for the SOCS1 gene and PTPN2 gene (S+P2) (IDT, GGAAACTTGGCCACTCTATG SEQ ID NO: 206). 100 μM solution of OR1A2 sgRNA was made by resuspending 10 nmol lyophilized sgRNA with 100 μl Nuclease Free Duplex Buffer (IDT Cat #1072570). Reagents were added as follows:

| | Vol (μL) |
|---|---|
| sgRNA (100 μM) | 1.2 |
| Cas9 (61 μM) | 0.84 |
| DPBS (1X) | 1.76 |

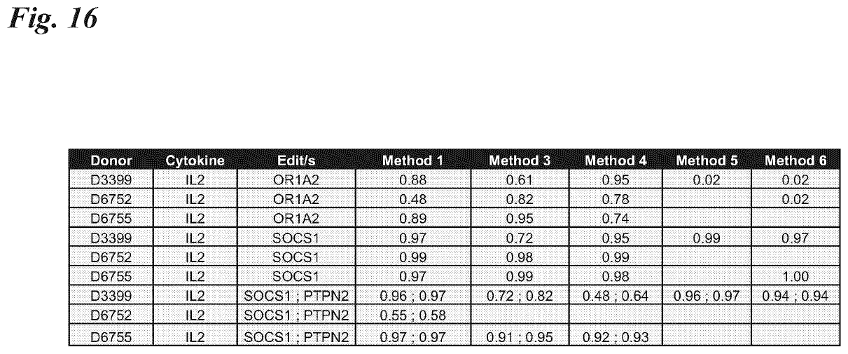

The entire 5 μl of the RNP master mix was added to the 20 μl cell suspension. 25 μl of cell suspension was then transferred to an OC25×3 processing assembly (MaxCyte, Cat #OC-25×3). Cells were electroporated on a MaxCyte ExPERT electroporator using the “Optimization #9” program. Subsequently, 25 μl TIL were transferred to a 96-well plate, each chamber was washed with 25 μL TIL media twice and transferred to the 96-well recovery plate, which was then incubated at 37° C. for 20 minutes. Subsequently, cells were counted, and 2×10⁵ live cells were then seeded into a 24 well Grex plate containing 6 ml TIL media supplemented with 6,000 U/ml IL-2. Further cell manipulations were conducted beginning on day 13 as described in Example 5. On days 18 and 23, cells were harvested and counted. Cell pellets were frozen, and editing was determined by amplicon sequencing (FIG. 16).

### Example 7: Phenotypic Characterization of Pre-REP Failure TILs with Soluble Activators or aAPCs

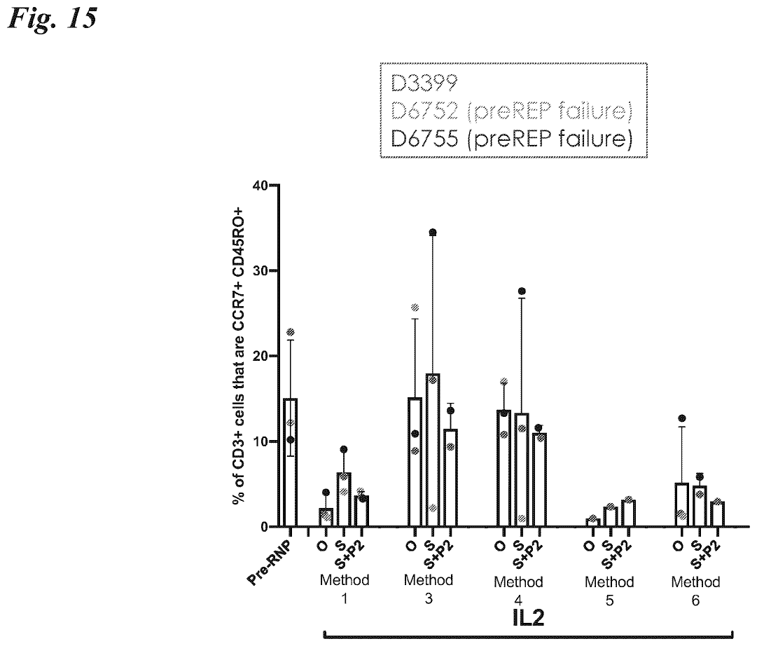

The phenotype of T cells produced on day 18 or 23 was assessed. In particular, the proportion of cells that were defined as central memory T cell phenotype (T_{cm}, with marker phenotype CD45RO+ CCR7+ CD45RA−) was determined by flow cytometry. Cells cultured as in Example 5 were taken, and on day 18 or 23 an aliquot of cells was stained with fluorescently labeled antibodies that detect CD45RO, CCR7, and CD45RA. Compared to pre-RNP (cells prior to electroporation) Method 3 (Stemcell) and Method 4 (Transact) generated similar percentages of T_{cm} cells on day 18 or 23 (FIG. 15). The percentage of CD8+ T cells showed a general enrichment as compared to pre-RNP cells for all methods at day 18 or 23 (data not shown).

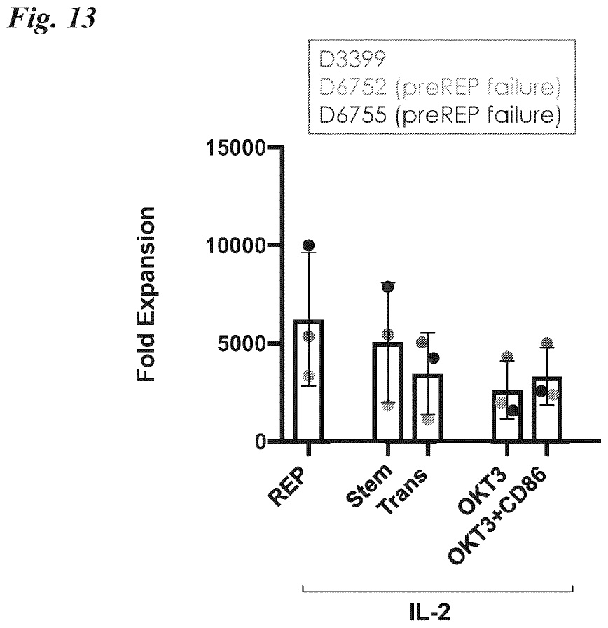

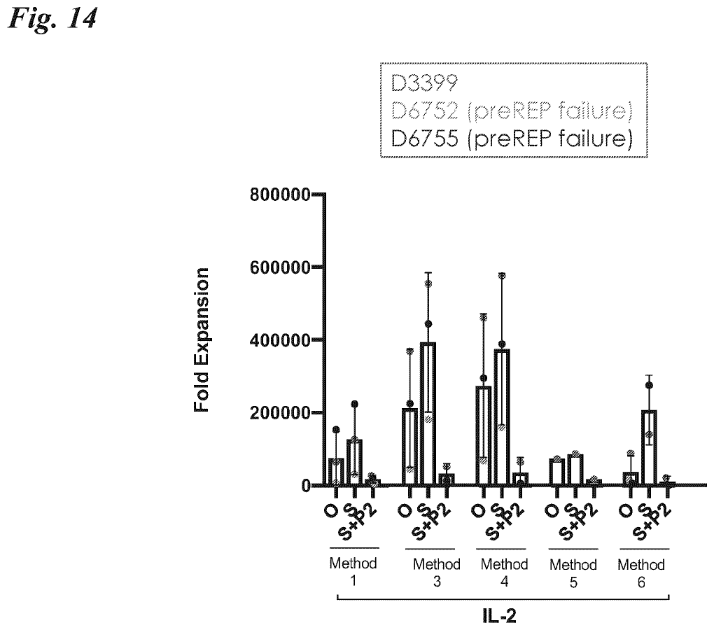

The fold expansion (FIG. 13) of TILs at day 10 or 11 (relative to the number of cells on Day 0) prior to electroporation was assessed for TILs expanded by the addition of IL-2 for the five methods described in Example 5. All donors, including the two pre-REP failures showed expansions greater than 2600 fold in methods 1, 3, 4, 5, and 6. SOCS1 edited TILs showed greater mean fold expansion than olfactory and SOCS1+PTPN2 edited TILs across all methods on days 18 or 23 (FIG. 14). Method 6 showed greater mean fold expansion of SOCS1 edited TIL as compared to Method 5 on day 18 or 23.

### Example 8: Methods for Expanding TILs from a Tumor Fragment Using a Soluble Activator

Tumor infiltrating lymphocytes (TILs) were expanded directly from frozen melanoma tumor fragments from primary patients. Tumor fragments were obtained from 2 donors: Donor D4462 and Donor D7283. On Day 0, tumor fragments were thawed and placed in a 10 cm² dish containing TIL media (a 1:1 mixture of RPMI 1640 and AIM V, supplemented with 5% human AB serum). Fragments were weighed and then evenly split (by number of fragments) into two aliquots and each aliquot was placed in a well of a 24 well Grex plate (Wilson Wolf, Cat #80192M). 6 mL of TIL media was added to each well containing a 1:70 dilution of GMP TransAct reagent (MACS GMP T Cell Transact, Cat #170-076-156) in 6000 U/mL IL-2 (Peprotech, Cat #200-02). Cells were cultured at 37° C.

On Day 2 of the expansion, 36,000 U of recombinant human IL-2 was added to each well, for a final concentration of 6,000 U/mL assuming consumption.

On Day 6 of the expansion, for D7283 a 50% media was replaced/exchanged. From each well, 3 mL of cell supernatant was removed and discarded, being careful not to disturb the cells at the bottom of the well. Subsequently, 3 mL of fresh TIL media and 36,000 U of IL-2 was then added to a final concentration of 6,000 U/ml assuming consumption. For D4462, samples were engineered using CRISPR-Cas9 as described in Example 9. Following electroporation, 4×10⁵ cells were transferred to a 24 well Grex (Wilson Wolf) in 6 mL TIL media containing 6,000 U/mL of IL-2.

On Day 9 of the expansion, a 50% media was replaced/exchanged. From each well, 3 mL of cell supernatant was removed and discarded, being careful not to disturb the cells at the bottom of the well. Subsequently, 3 mL of fresh TIL media and 36,000 U of IL-2 was then added, for a final concentration of 6,000 U/ml, assuming consumption.

On Day 10 of the expansion, for D4462, 3 mL of media was aspirated from each well of a 24 well Grex. The remaining 3 mL was added to a 6 well Grex (Wilson Wolf) containing 100 mL TIL media with 6,000 U/mL IL-2. For D7283, samples were engineered using CRISPR-Cas9 as described in Example 9. Following electroporation, 4×10⁵ cells were transferred to a 24 well Grex (Wilson Wolf) in 6 mL TIL media containing 6,000 U/mL.

On Day 14 of the expansion, D4462 wells were harvested. D7283, 3 mL of media was aspirated from each well of a 24 well Grex. The remaining 3 mL was added to a 6 well Grex (Wilson Wolf) containing 100 mL TIL media with 6,000 U/mL of IL-2.

On Day 17 of the expansion, D7283, 50 mL TIL media was removed and replaced with 50 mL fresh TIL media. 6,000 U/mL of IL-2 was added to consumption.

On Day 20 of the expansion, D7283 wells were harvested.

### Example 9: Methods to Genetically Engineer Fragment Expanded TILs with Soluble Activators Using CRISPR-Cas9

TILs expanded using the protocols described in Example 8 were genetically engineered using CRISPR-Cas9 to create functional genetic knockouts of a target gene. This genetic engineering was performed on day 6 or day 10. Briefly, on day 6 or 10, 1.2×10⁶ expanded TILs were centrifuged at 300×g for 5 minutes and resuspended with 20 μl of MaxCyte electroporation buffer (HyClone Cat #EPB1). Two ribonucleoprotein (RNP) master mixes were made containing 52 pmol Cas9 protein (Aldevron, Cat #9212) and 120 pmol of each individual sgRNA. Master mix 1 contained the sgRNA for the OR1A2 gene (O) (IDT, AGATGATGTCAACCAAGGAG SEQ ID NO: 910). Master mix 2 contained the sgRNA for the SOCS1 gene (S) (IDT, GACGCCTGCGGATTCTACTG SEQ ID NO: 61). 100 μM solution of OR1A2 sgRNA was made by resuspending 10 nmol lyophilized sgRNA with 100 μL Nuclease Free Duplex Buffer (IDT Cat #1072570). Reagents were added as follows:

| | Vol (μL) |
|---|---|
| sgRNA (100 μM) | 1.2 |
| Cas9 (61 μM) | 0.84 |
| DPBS (1X) | 1.76 |

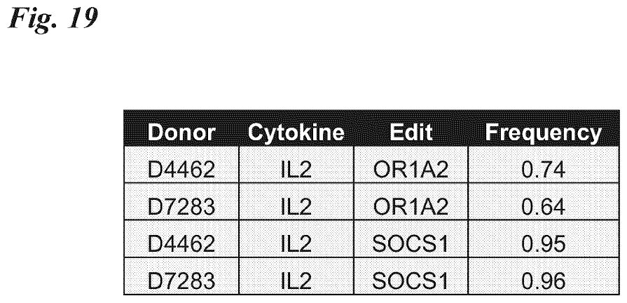

The entire 5 μL of the RNP master mix was added to the 20 μL cell suspension. μL of cell suspension was then transferred to an OC25×3 processing assembly (MaxCyte, Cat #OC-25×3). Cells were electroporated on a MaxCyte ExPERT electroporator using the “Optimization #9” program. Subsequently, 25 μL TIL were transferred to a 96-well plate, each chamber was washed with 25 μL TIL media twice and transferred to the 96-well recovery plate, which was then incubated at 37° C. for 20 minutes. Subsequently, cells were counted, and 4×10⁵ live cells were then seeded into a 24 well Grex plate containing 6 mL TIL media supplemented with 6,000 U/ml of IL-2. Further cell manipulations were conducted as described in Example 8. On days 14 and 20, cells were harvested and counted. Cell pellets were frozen, and editing was determined by NGS sequencing (FIG. 19).

### Example 10: Phenotypic Characterization of Tumor Fragment Expanded TILs with Soluble Activators or aAPCs

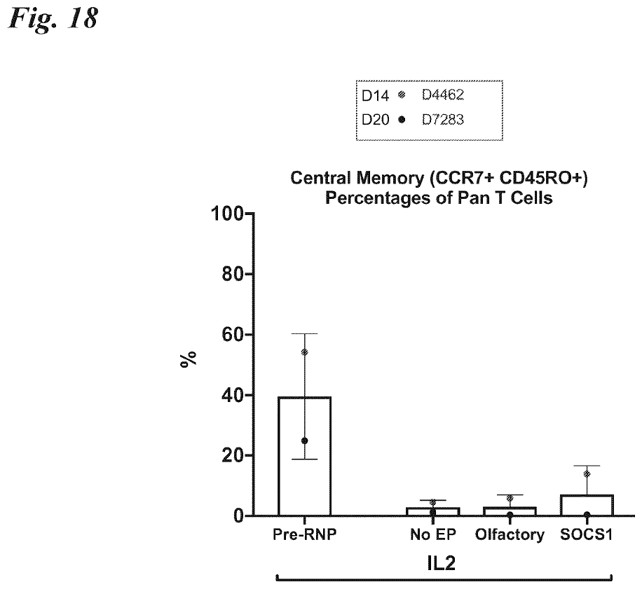

The phenotype of T cells produced on day 14 or 20 was assessed. In particular, the proportion of cells that were defined as central memory T cell phenotype (Tem, with marker phenotype CD45RO+ CCR7+ CD45RA−) or effector memory T cell phenotype (Teff, with marker phenotype CD45RO+ CCR7− CD45RA−) was determined by flow cytometry. Cells cultured as in Example 8 were taken, and on day 14 or 20 an aliquot of cells was stained with fluorescently labeled antibodies that detect CD45RO, CCR7, and CD45RA. All conditions tested showed predominantly a Teff memory phenotype. SOCS1 editing modestly increased Tcm phenotype (FIG. 18).

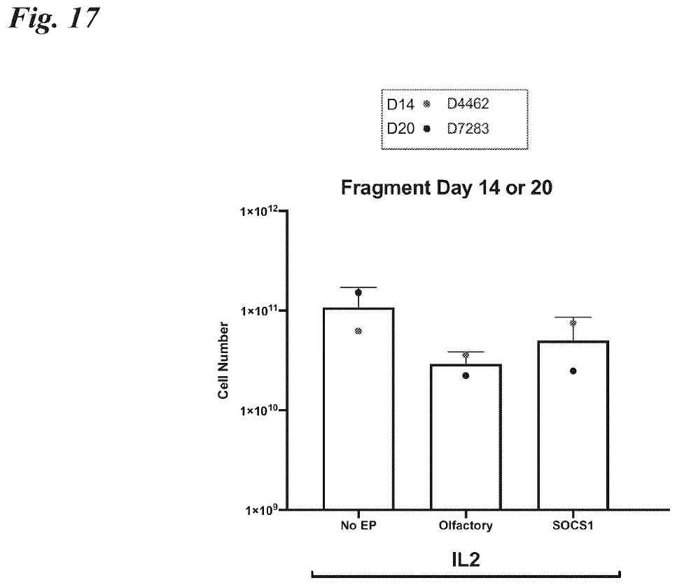

The theoretical TIL cell numbers generated by the soluble activator tumor fragment expansion methods at day 14 or 20 was assessed for TILs expanded by the addition of IL-2. Theoretical cell counts were calculated assuming a one gram tumor fragment sample. All conditions tested showed mean expansions greater than 1×10¹⁰ TIL after 20 days (FIG. 17).

### Example 11: Frozen Tumor Digest TIL Expansion and Frozen Tumor Fragment TIL Expansion

Frozen tumor digest TIL expansion was compared to frozen tumor fragment TIL expansion in the presence of IL-2 utilizing the TransACT activator. Following activation, editing for olfactory (O) and SOCS1 (S) was performed and compared to a no electroporation (no EP) control.

The materials used for this assessment were the following:

| | | |
|---|---|---|
| Human IL-2 (Lot 031912-1) | Peprotech | 200-02 |
| GMP T Cell TransAct (Lot 5200103426) | MACS | 170-076-156 |
| sNLS-SpCas9-sNLS Nuclease (Lot MPM030-04) | Aldevron | 9212-5MG |
| sg_Nh.SOCS1_u728 (custom A. Hohmann) | Biospring | 28May19 |
| sgOR1A2_axfc | IDT | 145243571. |
| Electroporation Buffer (Lot AD22541263, PD | Hyclone | EPB1 |
| 10/2018) | | |
| OC25x3 Processing Assembly (Lot LM236738) | MaxCyte | #OC-25x3 |
| Grex 24 well plate | Wilson Wolf | 80192M |
| Grex 6 well plate | Wilson Wolf | 80660M |
| AccuCheck Counting Beads | Life Technologies | PCB 100 |
| Cryostor CS10 | Stemcell Inc | 07930 |
| RPMI1640 1X L-Glutamine | Gibco | 11875-093 |
| AIM-V Medium 1X. | Gibco | 12055-091 |
| Human AB Serum- Sterile Filtered Heat Inactivated | Valley Biomedical | HP1022HI |

*Complete media: 50/50 RPMI/AIM-V, 5% human serum

Melanoma digests were received from Conversant Bio and melanoma tumor fragments were received from iSpecimen. The donor information and references were the following:
D3239 (Digest) BTC1000_
D6138 (Digest) BTC1000
D6755 (Digest) BTC1000
D4462 (Fragment) Melanoma_
D7283 (Fragment)_Melanoma_

For both TIL expansions, a common protocol was followed at discrete time intervals as indicated below:

At Day 0 of the expansion, cells were thawed according to Discovery Life Sciences Protocol (Thawing Viable Cell Products-1.pdf) using three vials per donor. Each TIL donor tube were resuspended in 1 mL complete media and combined for a total of 3 mL. The cells were counted using the Nexelcom Cellometer as per manufacturer's recommendations. 200 μL was removed from each donor for FACS staining. WI-002 ACT FACS Differentiation Panel.docx work instruction was followed for the staining. At the final resuspension step, 100 μL of an Accucheck beads solution was added (stock concentration 200,000 beads/mL) to obtain a total of 20,000 beads. The total numbers of T cells was calculated based upon acquired beads. Afterward, a TransAct reagent from a 2× working solution (1:35) was prepared to a final concentration of 1:70. 2×10⁶ cells and 3 mL of the 2× TransAct reagent were added to a well in a 24 well Grex, and the remaining TIL media was added to the cell to bring the total volume to 6 mL. IL-2 was added at a final concentration of 6,000 U/mL. The cells were incubated at 37° C.

Still at Day 0 of the expansion, the tumor fragment vials were thawed in a 37° C. water bath. The fragments were then poured into a 10 cm² dish containing 10 mL TIL media. The 10 cm² dish was placed on a measuring pad and the fragments were photographed. The fragments were split into two equal aliquots and each aliquot was placed into a 1.5 mL Eppendorf tube containing 1 mL TIL media. The fragments were spun down at 200 g for 5 minutes. The media was aspirated and the pooled fragments were weighted. 3 mL of the 2× TransACT reagent and 3 mL of the TIL media were added to wells of a 24 well Grex. Fragments were added to the wells of a 24 well Grex. For D4462, 8 fragments were combined with IL-2. For D7283, 6 fragments were combined with IL-2. IL-2 was added at 6,000 U/mL. The cells were incubated at 37° C.

At Day 2 of the expansion, IL-2 was added to all donors. IL-2 was added to consumption to 6,000 U/mL.

At Day 4 of the expansion, media for all donors were exchanged. 3 mL of media from each well was discarded and 3 mL of TIL media was added to each well. Afterward, IL-2 was added to a final concentration of 6,000 U/mL.

At Day 6 of the expansion, the D3239, D6138, D6755, and D4462 were FACS stained and electroporated. The concentration of olfactory sgRNA was adjusted to 100 μM by resuspending 10 nmol vial with 100 uL duplex buffer. The SOCS1 guide was already at the necessary concentration. A RNP solution for a total of 15 tests was prepared with the volumes below:

| | Vol/test (μL) |
|---|---|
| sgRNA (100 uM) | 1.2 |
| Cas9 (61 uM) | 0.84 |
| DPBS (1X) | 2.96 |
| RNP solution | 5 |
| Maxcyte sol (cells) | 20 |

The MaxCyte instrument was prepared and set to “optimization #9” OC25X3. 3 mL of media was aspirated from each well, the volume was recorded, and the cells were counted. 100 uL of pre-electroporated cells was transferred to a 96 well v-bottom plate and stained according to WI-002 ACT FACS Differentiation Panel.docx protocol. 1.2×10⁶ cells were added to a 1.5 mL Eppendorf tube for each condition. Tubes were spun down at 300 g for 5 minutes and the supernatant was removed. 20 uL of MaxCyte electroporation buffer was added to 1.5 mL Eppendorf tube. 5 uL of the Olfactory or SOCS1 RNP solution was added to the corresponding Eppendorf tube. Up to 25 μL was transferred to the OC25X3 assembly and the cells were electroporated. 25 μL of cells were transferred from a well from the OC25X3 to a 96 well recovery plate. The OC25X3 well was rinsed with 25 μL of TIL media two times for a final volume of 75 μL in the recovery plate well. The cells were incubated for 20 minutes at 37° C. The cells were counted by: adding 5 μL from the recovery plate to 45 uL TIL media in a counting well (10 fold dilution); adding 50 μL of AOPI and mixing; transferring to counting chamber; and counting the cells. 4×10⁵ cells were then transferred to a well of a 24 well Grex. The well was incubated at 37° C.

At Day 9 of the expansion, the media for all donors were exchanged. 3 mL of media was discarded from each well. 3 mL of TIL media was added to each well, and IL-2 was added to a final concentration of 6,000 U/mL.

At Day 10 of the expansion, the D7283 was FACS stained and electroporated. Samples were prepared as stated for the samples at Day 6. Enough was prepared for 5 samples.

Still at Day 10 of the expansion, samples D3239, D6138, D6755, and D4462 were transferred to a 6 well Grex. 100 mL of TIL media was added to a 6 well Grex containing 6,000 U/mL of IL-2. 3 mL of media from each donor well was discarded. Cells were counted and the volume recorded. 3 mL of donor cells was added to the corresponding well in a 6 well Grex containing 100 mL TIL media with cytokine.

At Day 14 of the expansion, takedown assays were performed for D3239, D6183, D6755, and D4462. 80 mL was aspirated from each well of the 6 well Grex, mixed, and their volume recorded. One vial was saved for NGS processing: 1 million of cells were transferred to a 1.5 mL Eppendorf tube, and the tube was spun down at 300 g for 5 minutes. Supernatant was aspirated and the cells stored at −80° C. FACS analysis was preformed: 1 million cells per condition were transferred to a v-bottom or u-bottom 96 well plate for the differentiation and polyfunctional panel respectively. Cells were processed according to work instructions. The remaining cells were frozen: 50 million cell pellets were prepared; the cells were spun at 300 g for 5 minutes; the supernatant was aspirated; cryostore was added; the cells were resuspended to 50 million cells/mL; 1 mL was added to cryovial and placed in a coozie at −80° C. overnight before transfer to LN2.

At Day 14 of the expansion, the D7283 was transferred to a 6 well Grex. 100 mL of TIL media was added to a 6 well Grex containing 6,000 U/mL of IL-2. 3 mL of media was discarded from each donor well. The cells were counted and the volume was recorded. 3 mL of donor cells was added to the corresponding well in a 6 well Grex containing 100 mL TIL media with cytokine.

At Day 17 of the expansion, the cells from the sample D7283 were counted and a 50% media exchange was performed. 50 mL media was removed and the cells were counted. 50 mL of TIL media was added for a total of 100 mL. IL-2 to 6,000 U/mL was added assuming consumption.

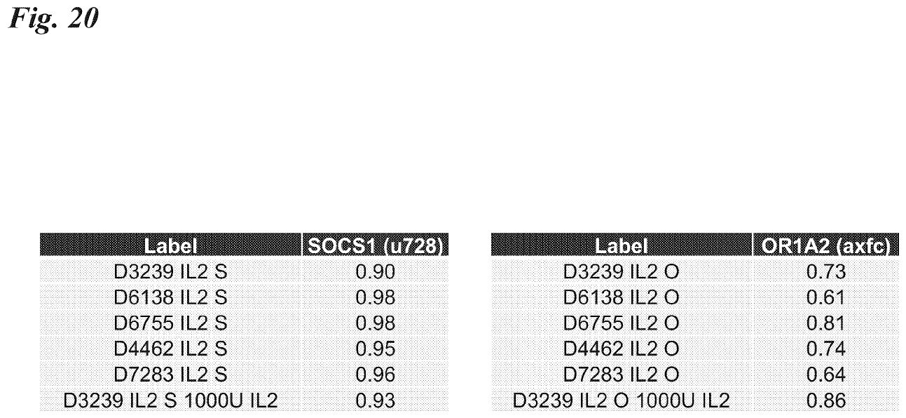

At Day 20 of the expansion, takedown assays were performed for D7283 and the expansion was continued. 70 mL from each well of the 6 well Grex was aspirated, mixed and their volume recorded. 5 million cells were removed to support takedown assays below. One vial for NGS processing was saved: 1 million of cells were transferred to a 1.5 mL Eppendorf tube and spun down at 300 g for 5 minutes; the supernatant was aspirated; and the was stored at −80° C. The editing efficiencies are depicted in FIG. 20. The FACS analysis was performed: 1 million of cells per condition were transferred to a v-bottom 96 well plate for the differentiation; and cells were processed according to work instruction. The remaining cells were frozen: 50 million cell pellets were prepared; the cells were spun at 300 g for 5 minutes; the supernatant was aspirated; cryostore was added and the cells were resuspended to 50 million cells/mL; and 1 mL was added to cryovial and placed in a coozie at −80° C. overnight before being transferred to LN2. 70 mL TIL media was added to wells for a total of 100 mL. IL-2 was added to 6,000 U/mL assuming consumption.

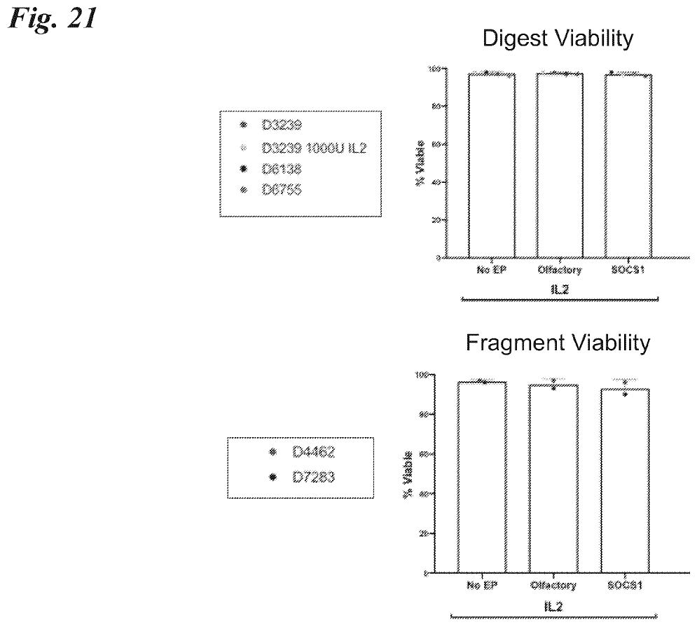

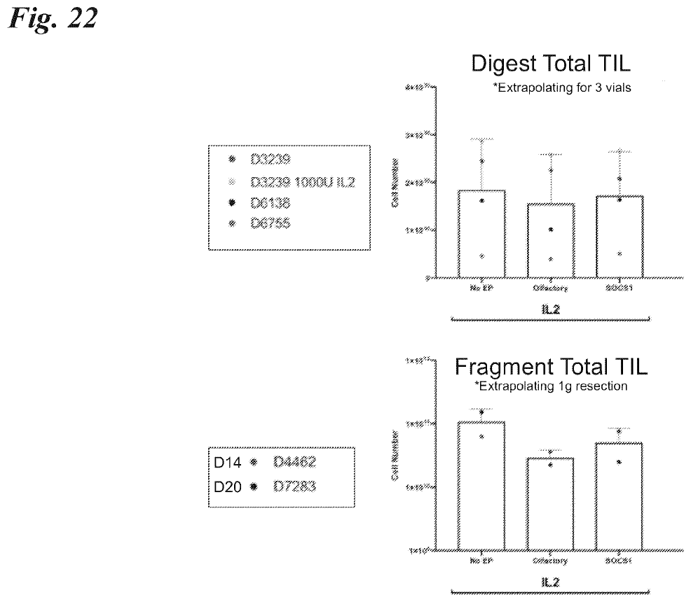

At Day 23 of the expansion, takedown assays were performed for D7283 and the sample was frozen down. 70 mL was aspirated from each well of the 6 well Grex, mixed, and their volume recorded. 1 million cells were removed to support takedown assays below. The FACS analysis was performed: 1 million cells per condition were transferred to a u-bottom 96 well plate for the polyfunctional pane; and cells were processed according to work instruction “WI-008 ACT FACS Polyfunctional Panel CD25 APC.docx.” The remaining cells were frozen: 50 million cell pellets were prepared; the cells were spun at 300 g for 5 minutes; the supernatant was aspirated; Cryostore was added and cells were resuspended to 50 million cells/mL. 1 mL was added to cryovial and placed in a coozie at −80° C. overnight before being transfer to LN2. TILs were determined to be highly viable (FIG. 21), with extrapolated TIL yields exceeding 1×10⁹ cells for both fragment and digest samples (FIG. 22) by Day 14 of the process.

1. A method of expanding a population of tumor infiltrating lymphocytes (TILs) in a disaggregated tumor sample, the method comprising culturing the disaggregated tumor sample in a medium, wherein the TILs are contacted with a T cell receptor (TCR) agonist, a CD28 agonist, and a T cell-stimulating cytokine.

2. The method of claim 1, wherein the medium is supplemented with the T cell-stimulating cytokine at a time interval selected from the group consisting of 1 day, 2 days, 3 days, 4 days, 5 days, and 6 days.

3. (canceled)

4. The method of claim 1, wherein the T cell-stimulating cytokine is IL-2.

5.-10. (canceled)

11. The method of claim 1, wherein the disaggregated tumor sample comprises digested tumor fragments.

12. The method of claim 1, wherein the medium comprises feeder cells.

13. The method of claim 12, wherein the feeder cells are peripheral blood mononuclear cells or antigen presenting cells.

14. The method of claim 12, wherein the feeder cells express the TCR agonist, the CD28 agonist and/or a 4-1BB ligand.

15. (canceled)

16. The method of claim 14, wherein the feeder cells are genetically modified to express the TCR agonist, the CD28 agonist and/or the 4-1BB ligand.

17. The method of claim 16, wherein the TCR agonist is a CD3 agonist and/or the CD28 agonist is CD86.

18. The method of claim 17, wherein the CD3 agonist is OKT3.

19. (canceled)

20. The method of claim 12, wherein the feeder cells are antigen presenting cells.

21. (canceled)

22. The method of claim 12, wherein the feeder cells are genetically modified to express the T cell-stimulating cytokine.

23. (canceled)

24. The method of claim 1, wherein the medium does not comprise feeder cells.

25.-26. (canceled)

27. The method of claim 1, wherein the TCR agonist and/or the CD28 agonist are linked to a nanomatrix comprising a colloidal suspension of matrices of polymer chains, wherein each nanomatrix is 1 to 500 nm in length in its largest dimension.

28. The method of claim 27, wherein the TCR agonist and the CD28 agonist are attached to the same polymer chains or to different polymer chains.

29.-30. (canceled)

31. The method of claim 1, wherein the TCR agonist comprises a soluble monospecific complex comprising two anti-CD3 antibodies linked together, and/or wherein the CD28 agonist comprises a soluble monospecific complex comprising two anti-CD28 antibodies linked together.

32. (canceled)

33. The method of claim 1, wherein the medium comprises a CD2 agonist.

34. The method of claim 33, wherein the CD2 agonist comprises a soluble monospecific complex comprising two anti-CD2 antibodies linked together.

35. A method for expanding a population of tumor infiltrating lymphocytes (TILs) comprising contacting the population of TILs with a nanomatrix comprising a colloidal suspension of matrices of polymer chains, wherein the matrices are attached to CD3 agonists and CD28 agonists, wherein the nanomatrix provides activation signals to the population of TILs, thereby activating and inducing the population of TILs to proliferate, wherein each matrix is 1 to 500 nm in length in its largest dimension, and wherein the method does not comprise the use of feeder cells during expansion of the population of TILs.

36.-52. (canceled)

53. A method for expanding a population of TILs comprising contacting the population of TILs with a composition comprising a first, a second, and a third soluble monospecific complex, wherein each soluble monospecific complex comprises two antibodies or fragments thereof linked together, wherein each antibody or fragments thereof of each soluble monospecific complex specifically binds to the same antigen on the population of TILs, wherein the first soluble monospecific complex comprises an anti-CD3 antibody, wherein the second soluble monospecific complex comprises an anti-CD28 antibody, and wherein the third soluble monospecific complex comprises an anti-CD2 antibody, and the method does not comprise the use of feeder cells during expansion of the population of TILs.

54.-104. (canceled)

105. A composition comprising an expanded population of TILs produced by the method of claim 1.
